(19) United States
(12) Patent Application Publication
(10) Pub. No.: US 2026/0167623 A1
(43) Pub. Date: Jun. 18, 2026

(54) BIFUNCTIONAL COMPOUNDS CONTAINING 2,5-SUBSTITUTED PYRIMIDINE DERIVATIVES FOR DEGRADING CYCLIN-DEPENDENT KINASE 2 VIA UBIQUITIN PROTEASOME PATHWAY

(71) Applicant: NIKANG THERAPEUTICS, INC., Wilmington (DE)

(72) Inventors: Zhiyong YU, Wilmington, DE (US); Yan LOU, Dallas, TX (US)

(21) Appl. No.: 19/124,239

(22) PCT Filed: Nov. 8, 2023
(86) PCT No.: PCT/US2023/079145

§ 371 Date: Apr. 24, 2025

Related U.S. Application Data
(60) Provisional application No. 63/424,871, filed on Nov. 11, 2022.
(60) Provisional application No. 63/485,255, filed on Feb. 15, 2023.
(60) Provisional application No. 63/512,594, filed on Jul. 7, 2023.
(60) Provisional application No. 63/528,596, filed on Jul. 24, 2023.

(51) Int. Cl. C07D 401/14; A61K 31/506; A61K 31/513; A61K 45/06; A61P 35/00; C07D 417/14; C07D 487/10

(52) U.S. Cl. CPC C07D 401/14; A61K 31/506; A61K 31/513; A61K 45/06; A61P 35/00; C07D 417/14; C07D 487/10

(57) ABSTRACT

The present disclosure provides certain bifunctional compounds that cause degradation of Cyclin-dependent kinase 2 (CDK2) via ubiquitin proteasome pathway and are therefore useful for the treatment of diseases mediated by CDK2. Also provided are pharmaceutical compositions containing such compounds and processes for preparing such compounds.

### CROSS REFERENCE TO RELATED APPLICATIONS

This PCT International Patent application claims the benefit of U.S. Provisional Application No. 63/424,871, filed on Nov. 11, 2022; U.S. Provisional Application No. 63/485,255, filed on Feb. 15, 2023; U.S. Provisional Application No. 63/512,594, filed on Jul. 7, 2023; and U.S. Provisional Application No. 63/528,596, filed on Jul. 24, 2023; the entire contents of each of these applications are hereby incorporated by reference.

### FIELD OF THE DISCLOSURE

The present disclosure provides certain bifunctional compounds containing 2,5-substituted pyrimidine derivatives that cause degradation of Cyclin-dependent kinase 2 (CDK2) via ubiquitin proteasome pathway and are therefore useful for the treatment of diseases mediated by CDK2. Also provided are pharmaceutical compositions containing such compounds and processes for preparing such compounds.

### BACKGROUND

Cyclin-dependent kinases (CDKs) are cellular kinases that are critical for orchestrating signaling events such as DNA replication and protein synthesis to ensure faithful eukaryotic cell division and proliferation. To date, at least twenty-one mammalian CDKs have been identified (Malumbres M. Genome Biol. (2014) 15:122). Among these CDKs, at least CDK1/Cyclin B, CDK2/Cyclin E, CDK2/Cyclin A, CDK4/Cyclin D, CDK6/Cyclin D complexes are known to be important regulators of cell cycle progression; while other CDKs are important in regulating gene transcription, DNA repair, differentiation, and apoptosis (see Morgan, D. O. Annu. Rev. Cell. Dev. Biol. (1997) 13:261-291).

Due to their roles in regulating cell cycle and other essential cellular processes, increased activity or temporally abnormal activation of CDKs has been shown to result in the development of various types of cancer. Human tumor development is commonly associated with alterations in either the CDK proteins themselves or their regulators (Cordon-Cardo C. Am. J. Pathol. (1995) 147:545-560; Karp J E, Broder S. Nat. Med. (1995) 1:309-320; Hall M, Peters G. Adv. Cancer Res. (1996) 68:67-108). For example, amplifications of the regulatory subunits of CDKs and cyclins, and mutation, gene deletion, or transcriptional silencing of endogenous CDK inhibitory regulators have been reported (Smalley et al. Cancer Res. (2008) 68:5743-52). A large body of research has established the role of these alterations in promoting tumorigenesis and progression. Thus, there has been great interest in the development of inhibitors of the Cyclin-dependent kinases (CDKs) for therapeutic purposes over the last two decades.

Selective CDK 4/6 inhibitors have changed the therapeutic management of hormone receptor-positive (HR+) metastatic breast cancer (MBC). Palbociclib, ribociclib, and abemaciclib, selective reversible inhibitors of CDK4 and CDK6, are approved for hormone receptor-positive (HR+) metastatic breast cancer in combination with endocrine therapies. Additional clinical trials with these CDK4/6 inhibitors are ongoing in both breast and other cancers, either as single agents or in combination with other therapeutics. (O'Leary et al. Nature Reviews (2016) 13:417-430). While CDK4/6 inhibitors have shown significant clinical efficacy in ER-positive metastatic breast cancer, the clinical benefit may be limited over time due to the development of primary or acquired resistance.

An important mechanism of resistance to CDK4/6 inhibitors is the abnormal activation of CDK2. It has been reported that high Cyclin E expression leads to overactivated CDK2/Cyclin E complex, which bypasses the requirement for CDK4/6 for cell cycle reentry (Asghar, U. et al. Clin. Cancer Res. (2017) 23:5561). In addition, it has been found that when CDK4/6 is inhibited, there is a noncanonical CDK2/cyclin D1 complex formation that promotes pRb phosphorylation recovery and drives cell cycle progression (Herrera-Abreu M T et al, Cancer Res. (2006) 15: 2301).

The CDK2/Cyclin E complex plays an important role in regulation of the G1/S transition, histone biosynthesis and centrosome duplication. Following the initial phosphorylation of Rb by CDK4/6/cyclin D, CDK2/Cyclin E further hyper-phosphorylates p-RB, releases E2F to transcribe genes required for S-phase entry. During S-phase, Cyclin E is degraded and CDK2 forms a complex with Cyclin A to promote phosphorylation of substrates that permit DNA replication and inactivation of E2F, for S-phase completion. (Asghar et al. Nat. Rev. Drug. Discov. (2015) 14:130-146). In addition to cyclin bindings, the activity of CDK2 is also tightly regulated through its interaction with negative regulators, such as p21 and p27. In response to mitogenic stimulation, which signals optimal environment for cell cycle, p21 and p27 are phosphorylated and degraded, releasing the break on CDK2/Cyclin activation.

Cyclin E, the regulatory cyclin for CDK2, is frequently overexpressed in cancer, and its overexpression correlates with poor prognosis. For example, Cyclin E amplification or overexpression has been shown to associate with poor outcomes in breast cancer (Keyomarsi et al., N Engl J Med. (2002) 347:1566-75). Cyclin E2 (CCNE2) overexpression is associated with endocrine resistance in breast cancer cells and CDK2 inhibition has been reported to restore sensitivity to tamoxifen or CDK4/6 inhibitors in tamoxifen-resistant and CCNE2 overexpressing cells. (Caldon et al., Mol Cancer Ther. (2012) 11:1488-99; Herrera-Abreu et al., Cancer Res. (2016) 76:2301-2313). Cyclin E amplification also reportedly contributes to trastuzumab resistance in HER2+ breast cancer. (Scaltriti et al. Proc Natl Acad Sci. (2011) 108:3761-6). Cyclin E overexpression has also been reported to play a role in basal-like and triple negative breast cancer (TNBC), as well as inflammatory breast cancer (Elsawaf Z. et al. Breast Care (2011) 6:273-278; Alexander A. et al. Oncotarget (2017) 8:14897-14911.)

Amplification or overexpression of cyclin E1 (CCNE1) is also frequently found in ovarian, gastric, endometrial, uterus, bladder, esophagus, prostate, lung and other types of cancers (Nakayama et al. Cancer (2010) 116:2621-34; Etemadmoghadam et al. Clin Cancer Res (2013) 19:5960-71; Au-Yeung et al. Clin. Cancer Res. (2017) 23:1862-1874; Ayhan et al. Modern Pathology (2017) 30:297-303; Ooi et al. Hum Pathol. (2017) 61:58-67; Noske et al. Oncotarget (2017) 8:14794-14805) and often correlates with poor clinical outcomes.

In some cancers, loss-of-function mutations in FBXW7, a component of SCF^{Fbw7}ubiquitin E3 ligase responsible for cyclin E degradation, also leads to cyclin E overexpression and CDK2 activation. Alternatively, certain cancer cells express a hyperactive, truncated form of cyclin E. In addition, cyclin A amplification and overexpression have also been reported in various cancers such as hepatocellular carcinomas, colorectal and breast cancers.

In contrast to the frequent upregulation of Cyclin E, the inhibitory regulators of CDK2, p21 and p27 are often abnormally downregulated in cancers. It is postulated that the loss or decrease of these key endogenous inhibitors leads to high and/or abnormal temporal activation of CDK2, thereby promoting oncogenic growth.

In addition, CDC25A and CDC25B, protein phosphatases responsible for the dephosphorylations that activate the CDK2, are overexpressed in various tumors. These various mechanisms of CDK2 activation have been validated using mouse cancer models. Furthermore, CDK2/cyclin E phosphorylates oncogenic Myc to oppose ras-induced senescence, highlighting the importance of CDK2 in myc/ras-induced tumorigenesis. Inactivation of CDK2 has been shown to be synthetically lethal to myc over-expressing cancer cells.

Recently, pharmacologic inhibition or genetic deletion of CDK2 was shown to preserve hearing function in animal models treated with cisplatin or noise (Teitz T et al. J Exp Med. 2018 Apr. 2; 215 (4): 1187-1203). Mechanistically, inhibition of CDK2 kinase activity reduces cisplatin-induced mitochondrial production of reactive oxygen species, thereby enhancing survival of inner ear cells. Therefore, in addition to anti-tumor therapies, CDK2 inhibition can also be used as a promising preventive treatment for noise-, cisplatin-, or antibiotic-induced or age-related hearing loss, for which no Food and Drug Administration-approved drugs are currently available.

Targeted protein degradation is emerging as a potential therapeutic modality which utilizes endogenous protein degradation systems (such as the ubiquitin-proteasome pathway or the lysosomes) to eliminate specific proteins (Dale et al., 2021; Li and Crews, 2022). Proteins targeted for degradation by the ubiquitin-proteasome system are first “tagged” with ubiquitin through the ubiquitination process and are later proteolyzed by the giant enzyme complex, proteasome. The ubiquitination process is a sophisticated posttranslational modification cascade, in which three enzymes (ubiquitin-activating E1, ubiquitin-conjugating E2, and ubiquitin-protein E3 ligase enzymes) work sequentially to attach ubiquitin to substrate proteins. As E3 ligases can directly bind to substrates and determine the specificity of ubiquitination, the E3 ubiquitin ligase is the most diverse component of the ubiquitin-proteasome system with roughly 600 members.

Proteolysis-targeting chimeric molecules (PROTACs) are bifunctional molecules comprised of target protein-recruitment moiety and a ligand for E3 ligase, connected by a biocompatible linker. PROTACs could bring the protein of interest and the E3 ligase into close proximity and induce ubiquitination and subsequent degradation of the target protein by proteasome. Compared to traditional small molecule drugs that typically bind disease-relevant proteins and inhibit their function, PROTACs display several unique and attractive features that make them desirable drug candidates. PROTACs have the ability to target previously undruggable proteins as they are not limited to binding of the catalytic domains. PROTACs have been shown to be more selective than their inhibitor counterparts, potentially reducing off-target toxicity. Moreover, PROTACs can perform multiple rounds of target ubiquitination and degradation. Due to this catalytic mode of action, PROTACs can function at sub-stoichiometric receptor occupancies. The E3 ligases used in PROTACs mainly include cereblon (CRBN), Von Hippel-Lindau-containing complex (VHL), inhibitor of apoptosis protein (IAP), and mouse double minute 2 (MDM2).

In contract to inhibition, removal of CDK2 protein would eliminate CDK2 activity as well as any protein interaction or scaffolding function of CDK2. Accordingly, there is a need for bifunctional molecules that could recruit CDK2 to a ubiquitin ligase, and thereby causing ubiquitylation and proteasomal degradation of CDK2. The present disclosure fulfills this and related needs.

### SUMMARY

In a first aspect, provided is a compound for use in the degradation and/or inhibition of CDK2 wherein the compound comprises a CDK2 binding moiety of Formula (A1):

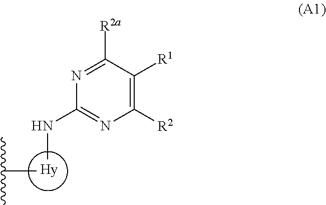

wherein:
R¹ is alkyl, alkenyl, alkynyl, alkylthio, pentafluorothio, halo, haloalkyl, haloalkylthio, haloalkoxy, alkoxy, amino, alkylamino, dialkylamino, cyano, cycloalkyl, cycloalkoxy, cycloalkylalkyl, bridged cycloalkyl, bridged cycloalkoxy, bridged cycloalkylalkyl, cyanoalkyl, cyanoalkoxy, alkoxyalkyl, aminoalkyl, aminoalkoxy, alkylaminoalkyl, dialkylaminoalkyl, alkylaminoalkoxy, dialkylaminoalkoxy, acyl, azidocarbonyl, alkoxycarbonyl, alkylcarbonylamino, aminocarbonyl, alkylaminocarbonyl, dialkylaminocarbonyl, aminosulfonyl, alkylaminosulfonyl, dialkylaminosulfonyl, substituted sulfonyl, substituted sulfinyl, substituted ureido, aryl, aralkyl, aryloxy, heteroaryl, heteroaralkyl, heteroaryloxy, heterocyclyl, heterocyclylalkyl, heterocyclyloxy, fused heterocyclyl, fused heterocyclyloxy, or fused heterocyclylalkyl, wherein cycloalkyl, by itself or as part of cycloalkoxy and cycloalkylalkyl, aryl, by itself or as part of aralkyl and aryloxy, heteroaryl, by itself or as part of heteroaralkyl and heteroaryloxy, heterocyclyl, by itself or as part of heterocyclylalkyl and heterocyclyloxy, bridged cycloalkyl, alone or as part of bridged cycloalkoxy and bridged cycloalkylalkyl, and fused heterocyclyl, by itself or as part of fused heterocyclylalkyl and fused heterocyclyloxy, are substituted with one, two, or three substituents independently selected from hydrogen, alkyl, alkoxy, halo, haloalkyl, haloalkoxy, and cyano;
R² and R^{2a} are independently hydrogen or deuterium; and
Hy is cycloalkylene, arylene, heteroarylene, heterocyclylene, bicyclic heterocyclylene, spiro heterocyclylene, bridged heterocyclylene, or fused heterocyclylene, where each of the aforementioned rings is substituted with R^{a}, R^{b}, and R^{e} independently selected from hydrogen, deuterium, alkyl, halo, haloalkyl, alkoxy, hydroxy, and cyano; or
   a pharmaceutically acceptable salt thereof;
wherein the compound of Formula (A1) degrades and/or inhibits CDK2 and the degradation of CDK2 is via ubiquitin proteasome pathway.

In a first embodiment of the first aspect, the compound of Formula (A1) degrades CDK2 via ubiquitin proteasome pathway.

In a second embodiment of the first aspect, the compound of Formula (A1) inhibits CDK2.

In a second aspect, provided is a compound for use in the degradation of CDK2 wherein the compound comprises a CDK2 binding moiety of Formula (A):

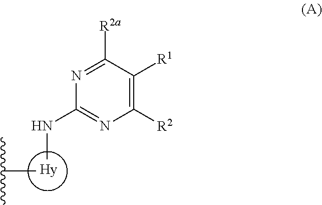

wherein:
R¹ is alkyl, alkenyl, alkynyl, cycloalkyl, halo, haloalkyl, haloalkoxy, alkoxy, aryloxy, cyano, or cycloalkyl where the cycloalkyl is substituted with one to three halo;
R² and R^{2a} are independently hydrogen or deuterium; and
Hy is cycloalkylene, arylene, heteroarylene, heterocyclylene, bicyclic heterocyclylene, spiro heterocyclylene, bridged heterocyclylene, or fused heterocyclylene, where each of the aforementioned rings is substituted with R^{a}, R^{b}, and R^{c} independently selected from hydrogen, deuterium, alkyl, halo, haloalkyl, alkoxy, hydroxy, and cyano; or
   a pharmaceutically acceptable salt thereof;
   wherein the degradation of CDK2 is via ubiquitin proteasome pathway.

In a third aspect, the compound of the of Formulae (A1) and (A) for use in the degradation of CDK2 as described in the first and/or second aspects are according to Formula (I):

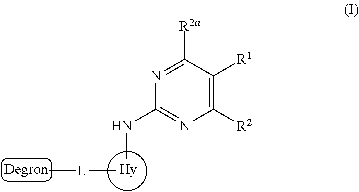

wherein:
R¹ is as defined therein;
R² and R^{2a} are independently hydrogen or deuterium;
Hy is cycloalkylene, arylene, heteroarylene, heterocyclylene, bicyclic heterocyclylene, spiro heterocyclylene, bridged heterocyclylene, or fused heterocyclylene, where each of the aforementioned rings is substituted with R^{a}, R^{b}, and R^{c} independently selected from hydrogen, deuterium, alkyl, halo, haloalkyl, alkoxy, hydroxy, and cyano;
L is a linker; and
Degron is an E3 ubiquitin ligase ligand; or
   a pharmaceutically acceptable salt thereof.

In a fourth aspect, provided is a compound of Formula (Ia):

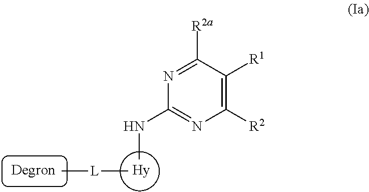

wherein
R¹ is alkyl, alkenyl, alkynyl, alkylthio, pentafluorothio, halo, haloalkyl, haloalkylthio, haloalkoxy, alkoxy, amino, alkylamino, dialkylamino, cyano, cycloalkyl, cycloalkoxy, cycloalkylalkyl, bridged cycloalkyl, bridged cycloalkoxy, bridged cycloalkylalkyl, cyanoalkyl, cyanoalkoxy, alkoxyalkyl, aminoalkyl, aminoalkoxy, alkylaminoalkyl, dialkylaminoalkyl, alkylaminoalkoxy, dialkylaminoalkoxy, acyl, azidocarbonyl, alkoxycarbonyl, alkylcarbonylamino, aminocarbonyl, alkylaminocarbonyl, dialkylaminocarbonyl, aminosulfonyl, alkylaminosulfonyl, dialkylaminosulfonyl, substituted sulfonyl, substituted sulfinyl, substituted ureido, aryl, aralkyl, aryloxy, heteroaryl, heteroaralkyl, heteroaryloxy, heterocyclyl, heterocyclylalkyl, heterocyclyloxy, fused heterocyclyl, fused heterocyclyloxy, or fused heterocyclylalkyl, wherein cycloalkyl, by itself or as part of cycloalkoxy and cycloalkylalkyl, aryl, by itself or as part of aralkyl and aryloxy, heteroaryl, by itself or as part of heteroaralkyl and heteroaryloxy, heterocyclyl, by itself or as part of heterocyclylalkyl and heterocyclyloxy, bridged cycloalkyl, alone or as part of bridged cycloalkoxy and bridged cycloalkylalkyl, and fused heterocyclyl, by itself or as part of fused heterocyclylalkyl and fused heterocyclyloxy, are substituted with one, two, or three substituents independently selected from hydrogen, alkyl, alkoxy, halo, haloalkyl, haloalkoxy, and cyano;
R² and R^{2a} are independently hydrogen or deuterium; and
Hy is cycloalkylene, arylene, heteroarylene, heterocyclylene, bicyclic heterocyclylene, spiro heterocyclylene, bridged heterocyclylene, or fused heterocyclylene, where each of the aforementioned rings is substituted with R^{a}, R^{b}, and R^{c} independently selected from hydrogen, deuterium, alkyl, halo, haloalkyl, alkoxy, hydroxy, and cyano;
Degron is an E3 ubiquitin ligase ligand selected from:
(a) a group of formula (i):

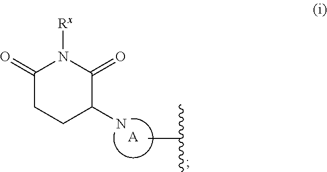

(b) a group of formula (ii):

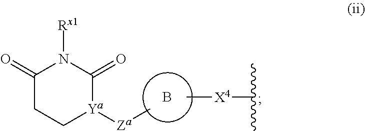

(c) a group of formula (iii):

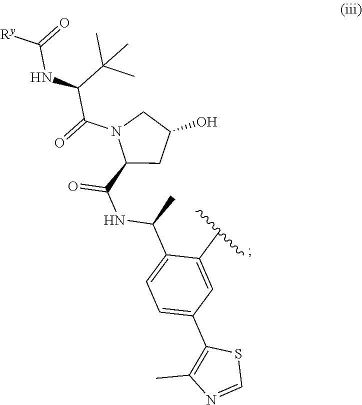

(d) a group of formula (iv):

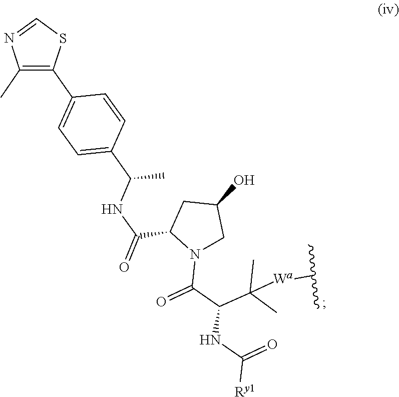

(e) a group of formula (v):

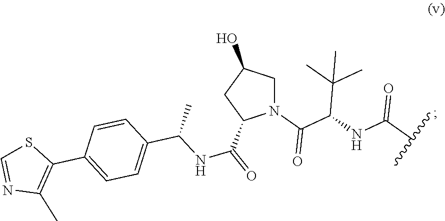 [SMILES: *C(=O)N[C@H](C(=O)N1C[C@H](O)C[C@H]1C(=O)N[C@@H](C)c1ccc(-c2scnc2C)cc1)C(C)(C)C]

and
(f) a group of formula (vi):

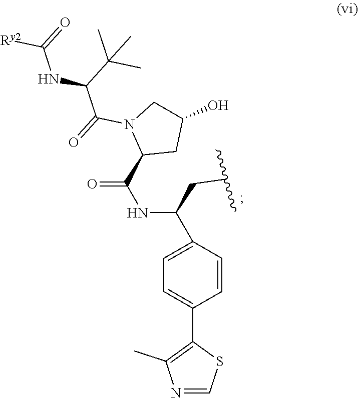

where:
   R^{x} and R^{x1} are each hydrogen;
   Y^{a} is CH or N;
   Z^{a} is a bond, —CH₂—, —NH—, —O—, or —NHC(O)— where NH of —NHC(O)— is attached to Y^{a};
   ring A is a group of formula (a), (b), or (c):

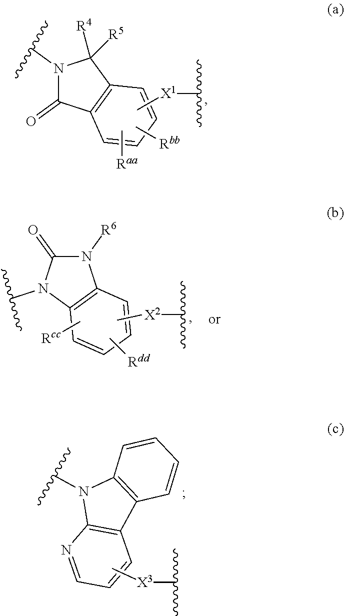

where:
   R^{aa}, R^{bb}, R^{cc}, and R^{dd} are independently selected from hydrogen, alkyl, alkoxy, halo, haloalkyl, haloalkoxy, and cyano;
   R⁴ and R⁵ are independently hydrogen or alkyl; or R⁴ and R⁵ together with the carbon to which they are attached form >C═O; and
   R⁶ is hydrogen or alkyl;
ring B is phenylene, cyclylaminylene, a 5- or 6-membered monocyclic heteroarylene, or a 9- or 10-membered fused bicyclic heteroarylene, wherein each heteroarylene ring contains one to three nitrogen ring atoms and further wherein the phenylene, cyclylaminylene, and each heteroarylene are independently substituted with R^{ee} and R^{ff} independently selected from hydrogen, alkyl, cycloalkyl, alkoxy, halo, haloalkyl, haloalkoxy, and cyano; and
X¹, X², X³, and X⁴ are independently a bond, -alkylene-, —O—, —(O-alkylene)-, (alkylene-O)—, —(NR^{gg}-alkylene)-, -(alkylene-NR^{hh})—,

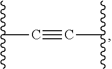 [SMILES: *C#C*]

—NH—, —N(alkyl)-, —C(═O)—, —NR^{jj}C(═O)—, or —C(═O)NR^{kk}— where R^{gg}, R^{hh}, R^{jj}, and R^{kk} are independently hydrogen, alkyl, or cycloalkyl and each alkylene, itself or as part of another group, is optionally substituted with one or two fluoro;
R^{y}, R^{y1}, and R^{y2} are independently alkyl, hydroxyalkyl, cycloalkyl or heterocyclyl wherein cycloalkyl and heterocyclyl are substituted with R^{d} and R^{f} selected from hydrogen, halo, cyano, alkylcarbonyl, and alkylcarbonylamino; and
W^{a} is bond, O, S, or alkylene; and
L is —Z¹—Z²—Z³—Z⁴—Z⁵—Z⁶— where:
Z¹ is a bond, alkylene, —C(O)NR—, —NR′(CO)—, —S(O)₂NR—, —NR′S(O)₂—, —(O-alkylene)ₐ-, -(alkylene-O)ₐ—, phenylene, monocyclic heteroarylene, or heterocyclylene, where each ring is substituted with R^{h} and Rⁱ independently selected from hydrogen, deuterium, alkyl, alkoxy, halo, haloalkyl, haloalkoxy, cyano, hydroxy, amino, alkylamino, and dialkylamino;
Z² is a bond, alkylene, alkynylene, —C(O)—, —C(O)N(R)—, —NR′(CO)—, —(O-alkylene)_{b}-, -(alkylene-O)_{c}—, —O(CH₂)₇—, —O(CH₂)₈—, cycloalkylene, or -heterocyclylene, where each ring is substituted with R^{j} and R^{k} independently selected from hydrogen, deuterium, alkyl, alkoxy, halo, haloalkyl, haloalkoxy, cyano, hydroxy, amino, alkylamino, and dialkylamino;
Z³ is a bond, alkylene, alkynylene, —C(O)NR—, —NR′(CO)—, —O—, —NR″—, —(O-alkylene)_{c}-, -(alkylene-O)_{c}—, cycloalkylene, spiro cyclolalkylene, phenylene, -(alkylene)-phenylene-, -phenylene-(alkylene)-, monocyclic heteroarylene, -(alkylene)-monocyclic heteroarylene-, -monocyclic heteroarylene-(alkylene)-, heterocyclylene, -(alkylene)-heterocyclylene-, -heterocyclylene-(alkylene)-, bicyclic heterocyclylene, bridged heterocyclylene, -(alkylene)-bridged heterocyclylene-, -bridged heterocyclylene-(alkylene)-, fused heterocyclylene, spiro heterocyclylene, -(alkylene)-spiro heterocyclylene-, -spiro heterocyclylene-(alkylene)-, or 11 to 13 membered spiro heterocyclylene, where each ring, by itself or as part of another group, is substituted with R^{m} and Rⁿ independently selected from hydrogen, deuterium, alkyl, alkoxy, halo, haloalkyl, haloalkoxy, cyano, hydroxy, amino, alkylamino, and dialkylamino;
Z⁴ is a bond, alkylene, alkynylene, -(alkylene-NR″)—, —(NR″-alkylene)-, —O—, —C(O)—, —NR″—, —(O-alkylene)_{d}-, -(alkylene-O)_{d}—, cycloalkylene, -(alkylene)-cycloalkylene-, -cycloalkylene-(alkylene)-, spiro cyclolalkylene, phenylene, heteroarylene, heterocyclylene, -(alkylene)-heterocyclylene-, -heterocyclylene-(alkylene)-, fused heterocyclylene, bridged heterocyclylene, -(alkylene)-bridged heterocyclylene-, -bridged heterocyclylene-(alkylene)-, spiro heterocyclylene,
(alkylene)-spiro heterocyclylene, or -spiro heterocyclylene-(alkylene)-, where each ring, by itself or as part of another group, is substituted with R^{o} and R^{p} independently selected from hydrogen, deuterium, alkyl, alkoxy, halo, haloalkyl, haloalkoxy, cyano, hydroxy, amino, alkylamino, and dialkylamino;
Z⁵ is a bond, -alkylene, —NR″—, —O—, —C(O)—, —S(O)₂—, —NR′(CO)—, —C(O)NR—, phenylene, monocyclic heteroarylene, or heterocycylene, where each ring is substituted with R^{q} and R^{r} independently selected from hydrogen, deuterium, alkyl, alkoxy, halo, haloalkyl, haloalkoxy, cyano, hydroxy, amino, alkylamino, and dialkylamino; and
Z⁶ is a bond, alkylene, —NR″—, —O—, -(alkylene-O)—, —C(O)—, —S(O)₂—, —NR′(CO)—, or —C(O)NR—;
where each R, R′ and R″ is independently hydrogen or alkyl, each a, b, c, and d is independently an integer selected from 1 to 6, and each alkylene of —Z¹—, —Z²—, —Z³—, —Z⁴—, —Z⁵— and —Z⁶—, by itself or as part of another group, is independently substituted with R^{s} and R^{t} where R^{s} is hydrogen or deuterium and R^{t} is hydrogen, deuterium, haloalkyl, hydroxy, alkoxy, cyano, cycloalkyl, heterocyclyl, aryl, or monocyclic heteroaryl, wherein cycloalkyl, heterocyclyl, aryl, monocyclic heteroaryl are substituted with one or two substituents independently selected from hydrogen, alkyl, alkoxy, halo, haloalkyl, haloalkoxy, or cyano; provided that at least one of —Z¹—Z²—Z³—Z⁴—Z⁵—Z⁶— is not a bond;
or a pharmaceutically acceptable salt thereof.

In a fifth aspect, provided is a method of treating a disease mediated by CDK2 in a patient, preferably the patient is in need of such treatment, which method comprises administering to the patient, preferably a patient in need of such treatment, a therapeutically effective amount of a compound of any of first, second, and third aspects or a compound of Formula (Ia) of the fourth aspect (or any of the embodiments thereof described herein), or a pharmaceutically acceptable salt thereof. (For the sake of clarity, the phrase “any of the embodiments thereof described herein” includes embodiments of the first, second, and third aspects and Formula (Ia) disclosed herein below, unless stated otherwise.) In a first embodiment of the fifth aspect, the disease is cancer. In a second embodiment of the fifth aspect the disease is cancer selected from lung cancer (e.g., adenocarcinoma, small cell lung cancer and non-small cell lung carcinomas, parvicellular and non-parvicellular carcinoma, bronchial carcinoma, bronchial adenoma, and/or pleuropulmonary blastoma), skin cancer (e.g., melanoma, squamous cell carcinoma, Kaposi sarcoma, and/or Merkel cell skin cancer), bladder cancer, breast cancer, cervical cancer, colorectal cancer, cancer of the small intestine, colon cancer, rectal cancer, cancer of the anus, endometrial cancer, gastric cancer, head and neck cancer (e.g., cancers of the larynx, hypopharynx, nasopharynx, oropharynx, lips, and/or mouth), liver cancer (e.g., hepatocellular carcinoma and/or cholangiocellular carcinoma), ovarian cancer, prostate cancer, testicular cancer, uterine cancer, esophageal cancer, gall bladder cancer, pancreatic cancer (e.g., exocrine pancreatic carcinoma), stomach cancer, thyroid cancer, and parathyroid cancer. In a third embodiment of the fifth aspect, the cancers are those that are resistant to CDK4/6 inhibitors through CDK2-mediated mechanisms e.g., breast cancer. In a fourth embodiment of the fifth aspect, the disease is an autoimmune disease or a condition associated with an autoimmune disease, which method comprises administering to the patient, preferably a patient in need of such treatment, a therapeutically effective amount of a compound of any one of first, second, and third aspects or a compound of Formula (Ia) (or any of the embodiments thereof described herein), or a pharmaceutically acceptable salt thereof. In some embodiments, the autoimmune disease or condition associated with an autoimmune disease is selected from rheumatoid arthritis (RA), systemic lupus erythematosus (SLE), primary Sjogren's syndrome (pSS), multiple sclerosis (MS), Crohn's disease (CD), uveitis, pemphigus vulgaris, and sepsis. In a fifth embodiment of the fifth aspect, the disease is gout. In a sixth embodiment of the fifth aspect, the therapeutically effective amount of a compound of Formulas (A1), (A), (I), or (Ia) (or any embodiment thereof disclosed herein including specific compounds), or a pharmaceutically acceptable salt thereof, is administered in a pharmaceutical composition.

In a sixth aspect, provided is a method of treating noise-induced, chemotherapy-induced (cisplatin-induced), antibiotic-induced, or age-related hearing loss, which method comprises administering to a patient, preferably a patient in need of such treatment, a therapeutically effective amount of a compound of any one of first, second, and third aspects, or a compound of Formula (Ia) of the fourth aspect (or any of the embodiments thereof described herein), or a pharmaceutically acceptable salt thereof. In some embodiments of the sixth aspect, the amount of hearing loss is reduced when compared to an age-matched control. In some embodiments, the hearing loss is prevented when compared to an age-matched control.

In a seventh aspect, provided is a pharmaceutical composition comprising a compound of any one of first, second, and third aspects or a compound of Formula (Ia) of the fourth aspect (or any of the embodiments thereof described herein), or a pharmaceutically acceptable salt thereof; and a pharmaceutically acceptable excipient.

In an eighth aspect, provided is a compound of any one of first, second, and third aspects or a compound of Formula (Ia) of the fourth aspect (or any of the embodiments thereof described herein), or a pharmaceutically acceptable salt thereof for use as a medicament. In one embodiment of the eighth aspect, the compound of any one of first, second, and third aspects or Formula (Ia) of the fourth aspect (or any embodiments thereof disclosed herein), or a pharmaceutically acceptable salt thereof is useful for the treatment of one or more of diseases disclosed in the fifth and sixth aspects above.

In a ninth aspect, provided is the use of a compound any one of first, second and third aspects or a compound of Formula (Ia) of the fourth aspect (or any of the embodiments thereof described herein), or a pharmaceutically acceptable salt thereof, in the manufacture of a medicament for treating a disease in a patient in which the activity of CDK2 contributes to the pathology and/or symptoms of the disease. In one embodiment of the ninth aspect, the disease is one or more of diseases disclosed in the fourth or fifth aspects above.

In a tenth aspect, provided is a method of degrading CDK2 in a cell via ubiquitin proteasome pathway which method comprises contacting the cell with a compound comprising a 2,5-disubstituted pyrimidinyl moiety wherein said moiety binds to CDK2, or a pharmaceutically acceptable salt thereof. In one embodiment of the tenth aspect, the 2,5-disubstituted pyrimidinyl moiety is a moiety of Formula (Ia) of the fourth aspect (or embodiments thereof as disclosed herein, including specific compounds). In another embodiment of the tenth aspect the CDK2 is degraded in a cell in a patient.

In an embodiment of any one of above aspects, CDK2 is selectively degraded over CDK1. In another embodiment of any one of above aspects, CDK2 is selectively degraded over CDK1 and CDK4 and/or CDK6.

In the aforementioned aspects involving the treatment of cancer, further embodiments are provided comprising administering the compound of any one of first aspect, second aspect, third aspect, and Formula (Ia), or a pharmaceutically acceptable salt thereof (or any embodiments thereof disclosed herein) or the pharmaceutical composition of the seventh aspect, in combination with at least one additional anticancer agent. When combination therapy is used, the agents can be administered simultaneously or sequentially.

### DETAILED DESCRIPTION

### Definitions

Unless otherwise stated, the following terms used in the specification and claims are defined for the purposes of this Application and have the following meaning:

“Alkyl” means a linear or branched saturated monovalent hydrocarbon radical of one to six carbon atoms, e.g., methyl, ethyl, propyl, 2-propyl, butyl, pentyl, and the like. It will be recognized by a person skilled in the art that the term “alkyl” may include “alkylene” groups.

“Alkenyl” means a linear or branched monovalent hydrocarbon radical of two to six carbon atoms containing a double bond e.g., ethenyl, propenyl, 2-propenyl, butenyl, pentenyl, and the like.

“Alkynyl” means a linear or branched monovalent hydrocarbon radical of two to six carbon atoms containing a triple bond e.g., ethynyl, propynyl, 2-propynyl, butynyl, and the like.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkenylene” means a linear unsaturated divalent hydrocarbon radical of two to six carbon atoms or a branched unsaturated divalent hydrocarbon radical of three to six carbon atom containing a double bond, e.g., ethen-diyl, propen-diyl, 2-propen-diyl, buten-diyl, penten-diyl, and the like.

“Alkynylene” means a linear unsaturated divalent hydrocarbon radical of two to six carbon atoms or a branched unsaturated divalent hydrocarbon radical of three to six carbon atom containing a triple bond, e.g.

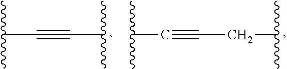

and the like.

“Alkoxy” means a —OR^{z} radical where R^{z} is alkyl as defined above, e.g., methoxy, ethoxy, propoxy, or 2-propoxy, n-, iso-, or tert-butoxy, and the like.

“Alkoxyalkyl” means alkyl as defined above that is substituted with alkoxy as defined above e.g., methoxymethyl, methoxyethyl, ethoxyethyl, and the like.

“Alkylthio” means an —SR^{z} radical where R^{z} is alkyl as defined above, e.g., methylthio, ethylthio, n-propylthio, 2-propylthio, n-, iso-, or tert-butylthio, and the like.

“Alkoxycarbonyl” means a —C(O)OR^{z} radical where R^{z} is alkyl as defined above, e.g., methoxycarbonyl, ethoxycarbonyl, and the like.

“Alkylcarbonyloxy” means an —OR^{z} group, where R^{z} is alkylcarbonyl, as defined herein.

“Alkylcarbonylamino” means a —NR^{z}′C(O)R^{z} radical where R^{z} is alkyl and R^{z} is H or alkyl, as defined above, e.g., methylcarbonylamino, ethylcarbonylamino, and the like.

“Acyl” means a —C(O)R^{z} radical where R^{z} is alkyl, haloalkyl, cycloalkyl, optionally substituted phenyl, optionally substituted heteroaryl, or optionally substituted heterocyclyl, as defined herein, e.g., methylcarbonyl, ethylcarbonyl, benzoyl, trifluoromethylcarbonyl, cyclopropylcarbonyl, and the like. When R^{z} is alkyl, acyl is also referred to herein as “alkylcarbonyl.”

“Azidocarbonyl” means —C(O)N² radical.

“Amido” means an —NR^{z}C(O)— or —C(O)NR^{z}— group, where R^{z} is hydrogen or alkyl as defined above.

“Sulfonamido” means an —NR^{z}S(O)₂— or —S(O)₂NR^{z}— group, where R^{z} is hydrogen or alkyl as defined above.

“Amino” means —NH₂.

“Aminoalkyl” means alkyl as defined above that is substituted with —NH₂ e.g., NH₂methyl, NH₂ethyl, and the like.

“Aminoalkyloxy” and “aminoalkoxy” mean —OR^{z} radical where R^{z} is aminoalkyl as defined above e.g., NH₂methyloxy, NH₂ethyloxy, and the like.

“Aminocarbonyl” means —C(O)NH₂.

“Alkylaminocarbonyl” means —C(O)NHR^{z} radical where R^{z} is alkyl as defined above e.g., methylaminocarbonyl, ethylaminocarbonyl, propylaminocarbonyl, and the like.

“Dialkylaminocarbonyl” means —C(O)NR^{z1}R^{z} radical where R^{z} and R^{z1} are independently alkyl as defined above e.g., dimethylaminocarbonyl, diethylaminocarbonyl, dipropylaminocarbonyl, and the like.

“Alkylamino” means —NHR^{z} radical where R^{z} is alkyl as defined above e.g., methylamino, ethylamino, propylamino, and the like.

“Aminosulfonyl” means —S(O)₂NH₂.

“Alkylaminosulfonyl” means —S(O)₂NHR^{z} radical where R^{z} is alkyl as defined above e.g., methylaminocarbonyl, ethylaminocarbonyl, propylaminocarbonyl, and the like.

“Dialkylaminosulfonyl” means —S(O)₂NR^{z1}R^{z} radical where R^{z} and R^{z1} are independently alkyl as defined above e.g., dimethylaminocarbonyl, diethylaminocarbonyl, dipropylaminocarbonyl, and the like.

“Alkylamino” means —NHR^{z} radical where R^{z} is alkyl as defined above e.g., methylamino, ethylamino, propylamino, and the like.

“Alkylaminoalkyl” means alkyl as defined above that is substituted with alkylamino as defined above e.g., methyaminomethyl, methylaminoethyl, ethylaminoethyl, and the like.

“Alkylaminoalkyloxy” means —OR^{z} radical where R^{z} is alkylaminoalkyl as defined above e.g., methyaminomethyloxy, methylaminoethyloxy, ethylaminoethyloxy, and the like.

“Aryl” means a monovalent monocyclic or bicyclic aromatic hydrocarbon radical of 6 to 10 ring atoms e.g., phenyl or naphthyl.

“Aralkyl” means an -(alkylene)-R^{z} radical where R^{z} is aryl as defined above e.g. benzyl.

“Arylene” means a divalent aryl (as defined above) radical e.g., phenylene or naphthylene.

“Aryloxy” means a —OR^{z} radical where R^{z} is aryl as defined above e.g., phenyloxy (or phenoxy), or naphthyloxy.

“Bicyclic heterocyclylene” means a saturated or unsaturated, divalent fused bicyclic group of 8 to 12 ring atoms in which one, two, or three ring atoms are heteroatoms independently selected from N, O, and S(O)ₙ, where n is an integer selected from 0 to 2, the remaining ring atoms being carbon, unless stated otherwise. Additionally, one or two ring carbon atoms of the bicyclic heterocyclylene ring can optionally be replaced by a —CO— group. More specifically the term bicyclic heterocyclylene includes, but is not limited to, isoindolin-diyl, decahydro-2,6-naphthyridin-diyl, octahydrocyclopenta[c]pyrrol-diyl, octahydro-1H-pyrrolo[3,4-c]pyridin-diyl, hexahydrofuro[3,2-b]furan-3,6-diyl, and the like. When the heterocyclylene ring is unsaturated it can contain one or two ring double bonds provided that the ring is not aromatic.

“Bridged cycloalkyl” means a saturated monovalent bicyclic ring having 5 to 8 ring carbon ring atoms in which two non-adjacent ring atoms are linked by a (CR^{z}R^{z}′)ₙ group where n is an integer selected from 1 to 3 and R^{z} and R^{z}′ are independently H or methyl (also may be referred to herein as “bridging” group). Examples include, but are not limited to, bicyclo[1.1.1]pent-1-yl, bicyclo[2.2.1]heptyl, preferably, bicyclo[2.2.1]hept-2-yl, and the like.

“Bridged cycloalkylalkyl” means a -(alkylene)-R^{z} radical where R^{z} is bridged cycloalkyl as defined above e.g., bicyclo[1.1.1]pent-1-ylmethyl, and the like.

“Bridged cycloalkyloxy” and “bridged cycloalkoxy” mean a —OR^{z} radical where R^{z} is bridged cycloalkyl as defined above e.g., bicyclo[2.2.1]hept-2-yloxy.

“Bridged cycloalkylene” means a saturated divalent bicyclic ring having 5 to 8 ring carbon ring atoms in which two non-adjacent ring atoms are linked by a (CR^{z}R^{z}′)ₙ group where n is an integer selected from 1 to 3 and R^{z} and R^{z}′ are independently H or methyl (also may be referred to herein as “bridging” group). Bridged cycloalkyl is optionally substituted with one or two substituents independently selected from alkyl, halo, alkoxy, hydroxy, and cyano unless stated otherwise. Examples include, but are not limited to, bicyclo[2.2.1]heptylene, preferably, bicyclo[2.2.1]hept-2,5-ylene.

“Bridged heterocyclylene” means a saturated divalent bicyclic ring having 5 to 9 ring carbon ring atoms in which two non-adjacent ring atoms are linked by a (CR^{z}R^{z}′)ₙ group where n is an integer selected from 1 to 3 and R^{z} and R^{z}′ are independently H or methyl (also may be referred to herein as “bridging” group) and further wherein one or two ring carbon atoms, including an atom in the bridging group, is replaced by a heteroatom selected from N, O, and S(O)ₙ, where n is an integer selected from 0 to 2. Bridged heterocyclylene is optionally substituted with one or two substituents independently selected from alkyl, halo, alkoxy, hydroxy, and cyano unless stated otherwise. Examples include, but are not limited to, 3,8-diazabicyclo[3.2.1]octa-3,8-diyl, 7-oxabicyclo[2.2.1]heptan-diyl, 2,5-diazabicyclo[2.2.1]heptan-diyl, 3,6-diazabicyclo-[3.1.1]heptan-diyl, 2,5-diazabicyclo[2.2.2]octan-diyl, 3,8-diazabicyclo[3.2.1]octan-diyl, 6-azabicyclo[3.1.1]heptan-diyl, 8-azabicyclo[3.2.1]octan-diyl, and the like.

“Cycloalkyl” means a monocyclic saturated monovalent hydrocarbon radical of three to ten carbon atoms. Examples include, but are not limited to, cyclopropyl, cyclobutyl, cyclopentyl, cyclohexyl, and the like.

“Cycloalkyloxy or cycloalkoxy” means a —OR^{z} radical where R^{z} is cycloalkyl as defined above. Examples include, but are not limited to, cyclopropyloxy, cyclobutyloxy, cyclopentyloxy, cyclohexyloxy, and the like.

“Cycloalkylalkyl” means an -(alkylene)-R^{z} radical where R^{z} is cycloalkyl as defined above. Examples include, but are not limited to, cyclopropylmethyl, cyclobutylethyl, cyclopentylmethyl, cyclohexylmethyl, and the like.

“Cycloalkylene” means a divalent saturated hydrocarbon radical of three to six carbon atoms, unless stated otherwise e.g., 1,1-cyclopropylene, 1,1-cyclobutylene, 1,4-cyclohexylene, and the like.

“Carbonyl” means —C(O)—.

“Carboxy” means —COOH.

“Cyclylaminylene” means a saturated divalent monocyclic ring of 4 to 8 ring atoms in which one or two ring atoms are nitrogen, the remaining ring atoms being carbon. More specifically, the term cyclylaminyl includes, but is not limited to, pyrrolidinylene, piperidinylene, homopiperidinylene, piperazinylene, and the like.

“Cyanoalkyl” means alkyl as defined above that is substituted with a cyano e.g., cyanomethyl, cyanoethyl, and the like.

“Cyanoalkoxy” means an —OR^{z} radical where R^{z} is cyanoalkyl as defined above. Examples include, but are not limited to, cyanomethoxy, cyanoethoxy, and the like.

“Deuterium” means refers to ²H or D.

“Deuteroalkyl” means alkyl as defined above, which is substituted with one, two, or three deuterium.

“Dialkylamino” means a —NR^{z}R^{z} radical where each R^{z} is alkyl as defined above, e.g., dimethylamino, methylethylamino, n-propylmethylamino, 2-propylmethylamino, n-, iso-, or tert-butylmethylamino, and the like.

“Dialkylaminoalkyl” means alkyl as defined above that is substituted with dialkylamino as defined above e.g., dimethyaminomethyl, dimethylaminoethyl, ethylmethylaminoethyl, and the like.

“Dialkylaminoalkyloxy” and “dialkylaminoalkoxy” mean —OR^{z} radical where R^{z} is dialkylaminoalkyl as defined above e.g., dimethyaminomethyloxy, dimethylaminoethyloxy, ethylmethylaminoethyloxy, and the like.

“Ether” means an —O— group.

“Fused heterocyclyl” means a monovalent bicyclic ring in which two adjacent ring atoms of a saturated or partially unsaturated (but not aromatic) monocyclic ring of 4 to 7 ring atoms having one or two heteroatoms independently selected from N, O, and S(O)ₙ (where n is 0, 1, or 2) and the remaining ring atoms being carbon, are fused to two adjacent ring members of a phenyl, or a five or six membered heteroaryl, each as defined herein, unless stated otherwise. The nitrogen atom is optionally oxidized or quaternized. The fused heterocyclylene can be attached at any atom of the ring. Representative examples include, but are not limited to, 1,2,3,4-tetrahydroquinolinyl, 3,4-dihydro-2H-benzo[b][1,4]oxazinyl, 3,4-dihydro-2H-pyrido[3,2-b][1,4]oxazinyl, 4,5,6,7-tetrahydropyrazolo[1,5-a]pyrazinyl, and the like.

“Fused heterocyclylene” means a divalent bicyclic ring in which two adjacent ring atoms of a saturated or partially unsaturated (but not aromatic) monocyclic ring of 4 to 7 ring atoms having one or two heteroatoms independently selected from N, O, and S(O)ₙ (where n is 0, 1, or 2) and the remaining ring atoms being carbon, are fused to two adjacent ring members of a phenyl, or a five or six membered heteroaryl, each as defined herein, unless stated otherwise. The nitrogen atom is optionally oxidized or quaternized. The fused heterocyclylene can be attached at any two atoms of the ring. Representative examples include, but are not limited to, 1,2,3,4-tetrahydroquinolin-1,4-diyl, 3,4-dihydro-2H-benzo[b][1,4]oxazin-5,8-diyl, 3,4-dihydro-2H-pyrido[3,2-b][1,4]oxazin-diyl, 4,5,6,7-tetrahydropyrazolo[1,5-a]pyrazin-diyl, and the like.

“Fused heterocyclylalkyl” means an -(alkylene)-R^{z} radical where R^{z} is fused heterocyclyl as defined above e.g., 1,2,3,4-tetrahydroquinolinylmethyl, 3,4-dihydro-2H-benzo[b][1,4]oxazinylmethyl, 3,4-dihydro-2H-pyrido[3,2-b][1,4]oxazinylmethyl, 4,5,6,7-tetrahydropyrazolo[1,5-a]pyrazinylmethyl, and the like.

“Fused heterocyclyloxy” means an —OR^{z} radical where R^{z} is fused heterocyclyl as defined above e.g., 1,2,3,4-tetrahydroquinolinyloxy, 3,4-dihydro-2H-benzo[b][1,4]oxazinyloxy, 3,4-dihydro-2H-pyrido[3,2-b][1,4]oxazinyloxy, 4,5,6,7-tetrahydropyrazolo[1,5-a]pyrazinyloxy, and the like.

“Halo” means fluoro, chloro, bromo, or iodo, preferably fluoro or chloro.

“Haloalkyl” means alkyl radical as defined above, which is substituted with one or more halogen atoms, e.g., one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this Application as fluoroalkyl.

“Haloalkoxy” means a —OR^{z} radical where R^{z} is haloalkyl as defined above e.g., —OCF₃, —OCHF₂, and the like. When R^{z} is haloalkyl where the alkyl is substituted with only fluoro (in some examples, one or more fluoro), it is referred to in this Application as fluoroalkoxy.

“Haloalkylthio” means an —SR^{z} radical where R^{z} is haloalkyl as defined above e.g., —SCF₃, —SCHF₂, and the like.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present, they are not both present on the same carbon atom. Representative examples include, but are not limited to, hydroxymethyl, 2-hydroxy-ethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxy butyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Heteroaryl” means a monovalent monocyclic or fused bicyclic aromatic radical of 5 to 10 ring atoms, unless otherwise stated, where one or more, (in one embodiment, one, two, or three), ring atoms are heteroatom selected from N, O, and S, the remaining ring atoms being carbon. Representative examples include, but are not limited to, pyrrolyl, thienyl, thiazolyl, imidazolyl, furanyl, indolyl, isoindolyl, indazolyl, imidazo[1,2-a]pyridinyl, imidazo[1,2-a]pyrazinyl, oxazolyl, isoxazolyl, oxadiazolyl, benzothiazolyl, benzoxazolyl, quinolinyl, isoquinolinyl, pyridinyl, pyrimidinyl, pyrazinyl, pyridazinyl, triazolyl, tetrazolyl, and the like. As defined herein, the terms “heteroaryl” and “aryl” are mutually exclusive. When the heteroaryl ring contains 5- or 6 ring atoms and is a monocyclic ring, it is also referred to herein as 5- or 6-membered monocyclic heteroaryl or monocyclic heteroarylene. When the heteroaryl ring contains 9- or 10 ring atoms and is a fused bicyclic ring, it is also referred to herein as 9- or 10-membered fused bicyclic heteroaryl.

“Heteroarylene” means a divalent heteroaryl radical as defined above, unless stated otherwise. Representative examples include, but are not limited to, benzimidazoldiyl e.g., benzimidazole-1,5-diyl, and the like. When the heteroarylene ring contains 5- or 6 ring atoms and is a monocyclic ring, it is also referred to herein as monocyclic heteroarylene or as 5- or 6-membered monocyclic heteroarylene e.g., pyrazolyl-1.4-diyl. When the heteroarylene ring contains 9- or 10 ring atoms and is a fused bicyclic ring, it is also referred to herein as 9- or 10-membered fused bicyclic heteroarylene.

“Heteroarylalkyl” or “heteroaralkyl” means an -(alkylene)-R^{z} radical where R^{z} is heteroaryl as, monocyclic group of 4 to 8 ring atoms in which one or two ring atoms are heteroatom independently selected from N, O, and S(O)ₙ, where n is an integer selected from 0 to 2, the remaining ring atoms being C, unless stated otherwise. Additionally, one or two ring carbon atoms in the heterocyclyl ring can optionally be replaced by a —CO— group. More specifically the term heterocyclyl includes, but is not limited to, azetidinyl, pyrrolidinyl, piperidinyl, homopiperidinyl, 2-oxopyrrolidinyl, 2-oxopiperidinyl, morpholinyl, piperazinyl, tetrahydro-furanyl, tetrahydro-pyranyl, thiomorpholinyl, and the like. When the heterocyclyl ring is unsaturated, it can contain one or two ring double bonds provided that the ring is not aromatic. When the heterocyclyl group contains at least one nitrogen atom, it is also referred to herein as heterocycloamino and is a subset of the heterocyclyl group.

“Heterocyclylalkyl” means an -(alkylene)-R^{z} radical where R^{z} is heterocyclyl as defined above e.g. piperidinylmethyl and piperazinylmethyl.

“Heterocyclyloxy” means an —OR^{z} radical where R^{z} is heterocyclyl as defined above e.g. 1-methylpyrrolidin-3-oxy, 1-methylpyrrolidin-2-oxy, piperidin-3-oxy, piperidin-4-oxy and the like.

“Heterocyclylene” means a saturated or unsaturated, divalent, monocyclic group of 4 to 8 ring atoms in which one or two ring atoms are heteroatom independently selected from N, O, and S(O)ₙ, where n is an integer selected from 0 to 2, the remaining ring atoms being C, unless stated otherwise. Additionally, one or two ring carbon atoms in the heterocyclylene ring can optionally be replaced by a —CO— group. More specifically, the term heterocyclylene includes, but is not limited to,

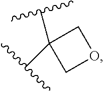 [SMILES: *C1(*)COC1]

piperidin-1,4-diyl, azetidin-1,3-diyl, and the like.

The term “Linker ‘L’” is a connector with a linear non-hydrogen atom number in the range of 1 to 20 (preferably, 2, 3, 4, 5, 6, 7, 8, 9, 10, 11, 12, 13, 14, 15, 16, 17, 18, 19, or 20; more preferably, 8 to 16, 9 to 14, 9 to 13, 9 to 12; more preferably 8, 9, 10, 11, 12, or 13; most preferably, 12 or 13). Linker “L” can contain one or more (preferably 2, 3, 4, 5, 6, 7, or 8; more preferably, 3 to 6 or 3, 4, 5, or 6; most preferably, 4 or 5), groups which are independently selected, such as, but not limited to, ether, polyether, thioether, —NH—, —N(alkyl)-, amido, sulfonamido, alkylene, alkenylene, alkynylene, carbonyl, —C(O)O—, —OC(O)—, sulfinyl, sulfonyl, ureido, thioureido, cycloalkylene, bridged cycloalkylene, spiro cycloalkylene, arylene, heteroarylene, heterocyclylene, bridged heterocycylene, spiro heterocyclylene, bicyclic heterocyclylene, or fused heterocyclylene, and wherein cycloalkylene, bridged cycloalkylene, spiro cycloalkylene, arylene, heteroarylene, heterocyclylene, bridged heterocycylene, spiro heterocyclylene, bicyclic heterocyclylene, and fused heterocyclylene are optionally substituted with one, two, or three substituents independently selected from deuterium, alkyl, alkoxy, halo, haloalkyl, haloalkoxy, hydroxy, cyano, amino, alkylamino, and dialkylamino, and wherein alkylene is optionally substituted with one or two halo (preferably fluoro). In some or any embodiments, Linker L contains 3 to 5 groups independently selected from —O—, —NH—, —N(CH₃)—, sulfonyl, phenylene, alkylene (preferably —CH₂—, —CH₂CH₂—, —CH₂CH₂CH₂—, —CH₂CH(CH₃)CH₂—, —CH(CH₃)CH₂—, —CH(CH₃)—, —CH₂C(CH₃)₂CH₂—, heterocyclylene (preferably azetidin-diyl, piperidin-diyl, or piperazin-diyl), spiro heterocyclylene (preferably 2,6-diazaspiro[3.3]heptan-diyl), and monocyclic heteroarylene (preferably, imidazolyl or pyridinyl; more preferably imidazolyl), wherein heterocyclylene, spiro heterocyclylene, and monocyclic heteroarylene are optionally substituted with one, two, or three substituents independently selected from halo (preferably fluoro) and alkyl (preferably methyl), and wherein alkylene is optionally substituted with one or two halo (preferably the group is —CH(CHF₂)—.

“Phenylene” means divalent phenyl.

“Polyether” means a

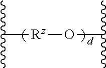

group where d is an integer selected from 2 to 5 and R^{z} is C₂₋₆alkylene.

The term “oxo,” as used herein, alone or in combination, means ═(O).

The phrase “optionally” or “optional” as used herein means that the subsequently described event or circumstance may but need not occur, and that the description includes instances where the event or circumstance occurs and instances in which it does not. For example, the phrase “alkylene optionally substituted with halo” is intended to cover alkylene that is unsubstituted and alkyene that is substituted with halo.

“Spiro cycloalkylene” means a saturated bicyclic divalent hydrocarbon ring having 6 to 12 ring atoms wherein the rings are connected through only one atom, the connecting atom is also called the spiroatom, most often a quaternary carbon (“spiro carbon”). Spiro cycloalkylene is optionally substituted with one or two substituents independently selected from alkyl, halo, alkoxy, hydroxy, and cyano, unless stated otherwise. Representative examples include, but are not limited to, spiro[3,5]nonandiyl e.g., spiro[3.5]nonane-2,7-diyl, and the like.

“Spiro heterocyclylene” means a saturated bicyclic divalent ring having 6 to 10 ring atoms in which one, two, or three ring atoms are heteroatom selected from N, O, and S(O)ₙ, where n is an integer selected from 0 to 2, the remaining ring atoms being C and the rings are connected through only one atom, the connecting atom is also called the spiroatom, most often a quaternary carbon (“spiro carbon”). Spiro heterocyclylene is optionally substituted with one or two substituents independently selected from alkyl, halo, alkoxy, hydroxy, and cyano, unless stated otherwise. Representative examples include, but are not limited to, 2-azaspiro[3.3]heptan-diyl, 2,6-diazaspiro[3.3]heptan-diyl, 1,7-diazaspiro[3.5]nonan-diyl, 2,7-diazaspiro[3.5]nonan-diyl, 3,9-diazaspiro[5.5]undecan-diyl, and the like.

“11 to 13 membered spiro heterocyclylene” means a saturated bicyclic divalent ring having 11 to 13 ring atoms in which one, two, or three ring atoms are heteroatom(s) selected from N, O, and S(O)ₙ, where n is an integer selected from 0 to 2, the remaining ring atoms being C and the rings are connected through only one atom, the connecting atom is also called the spiroatom, most often a quaternary carbon (“spiro carbon”). The 11 to 13 membered spiro heterocyclylene is optionally substituted with one or two substituents independently selected from alkyl, halo, alkoxy, hydroxy, and cyano, unless stated otherwise. Representative examples include, but are not limited to, diazaspiro[5.5]undecan-diyl, 1-oxa-diazaspiro[5.5]undecan-diyl, and the like.

“Pentafluorothio” means an —SF₅.

“Sulfinyl” means an —S(O)— group.

“Substituted sulfinyl” means an —S(O)R^{z} where R^{z} is alkyl as defined above e.g., methyl or ethylsulfinyl.

“Sulfonyl” means an —S(O)₂— group.

“Substituted sulfonyl” means an —S(O)₂R^{z} where R^{z} is alkyl as defined above e.g., methyl or ethylsulfonyl.

“Thioether” means an —S— group.

“Thioureido” means an —NHC(S)NH— group.

“Ureido” means an —NHC(O)NH— group.

“Substituted ureido” means an —NHC(O)NR^{z}R^{z′} where R^{z} is hydrogen or alkyl and R^{z′} is alkyl, as defined above e.g., —NHC(O)NHmethyl, —NHC(O)NMe₂, and the like.

The present disclosure also includes protected derivatives of compounds of first aspect, second aspect, third aspect, or Formula (Ia) (or any embodiments thereof disclosed herein), or a pharmaceutically acceptable salt thereof. For example, when compounds of Formula (Ia) contain groups such as hydroxy, carboxy, or any group containing a nitrogen atom(s), these groups can be protected with suitable protecting groups. A comprehensive list of suitable protective groups can be found in T. W. Greene, Protective Groups in Organic Synthesis, 5^{th} Ed., John Wiley & Sons, Inc. (2014), the disclosure of which is incorporated herein by reference in its entirety. The protected derivatives of compounds of the present disclosure can be prepared by methods well known in the art.

The present disclosure also includes polymorphic forms and deuterated forms of the compound of first aspect, second aspect, third aspect, or Formula (Ia) (or any embodiments thereof disclosed herein), or a pharmaceutically acceptable salt thereof.

The term “prodrug” refers to a compound that is made more active in vivo. Certain compounds Formulas (A1), (A), (I), or (Ia) (and any embodiment thereof disclosed herein including specific compounds) may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the active compound. Prodrugs are often useful because, in some situations, they may be easier to administer than the compound, or parent drug. They may, for instance, be bioavailable by oral administration whereas the parent drug is not. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

A “pharmaceutically acceptable salt” of a compound means a salt that is pharmaceutically acceptable and that possesses the desired pharmacological activity of the parent compound. Such salts include:
acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, PA, 1985, which is incorporated herein by reference in its entirety.

The compounds of Formulas (A1), (A), (I), or (Ia) (and any embodiment thereof disclosed herein including specific compounds) may have asymmetric centers. Compounds of Formulas (A1), (A), (I), or (Ia) (and any embodiment thereof disclosed herein including specific compounds) containing an asymmetrically substituted atom may be isolated in optically active or racemic forms. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. All chiral, diastereomeric, all mixtures of chiral or diastereomeric forms, and racemic forms are within the scope of this disclosure, unless the specific stereochemistry or isomeric form is specifically indicated. It will also be understood by a person of ordinary skill in the art that when a compound is denoted as (R) stereoisomer, it may contain the corresponding(S) stereoisomer as an impurity and vice versa.

Certain compounds of Formulas (A1), (A), (I), or (Ia) (and any embodiment thereof disclosed herein including specific compounds) can exist as tautomers and/or geometric isomers. All possible tautomers and cis and trans isomers, as individual forms and mixtures thereof are within the scope of this disclosure. Additionally, as used herein the term alkyl includes all the possible isomeric forms of said alkyl group albeit only a few examples are set forth. Furthermore, when the cyclic groups such as aryl is substituted, it includes all the positional isomers albeit only a few examples are set forth. Furthermore, all hydrates of a compound of Formulas (A1), (A), (I), or (Ia) (and any embodiment thereof disclosed herein including specific compounds) are within the scope of this disclosure.

The compounds of Formulas (A1), (A), (I), or (Ia) (and any embodiment thereof disclosed herein including specific compounds) may also contain unnatural amounts of isotopes at one or more of the atoms that constitute such compounds. Unnatural amounts of an isotope may be defined as ranging from the amount found in nature to an amount 100% of the atom in question. that differ only in the presence of one or more isotopically enriched atoms. Exemplary isotopes that can be incorporated into compounds of the present disclosure, such as a compound of Formulas (A1), (A), (I), or (Ia) (and any embodiment thereof disclosed herein including specific compounds) include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with (or isotopically enriched for) heavier isotopes such as deuterium (i.e., 2H) may afford certain therapeutic advantages resulting from greater metabolic stability (e.g., increased in vivo half-life or reduced dosage requirements). In some embodiments, in compounds of Formulas (A1), (A), (I), or (Ia) (and any embodiment thereof disclosed herein including specific compounds, including in Table 1 below, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁵F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use.

“A pharmaceutically acceptable carrier/excipient” as used in the specification and claims includes both one and more than one such excipient.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass ±10%, preferably ±5%, the recited value and the range is included.

Certain structures provided herein are drawn with one or more floating substituents. Unless provided otherwise or otherwise clear from the context, the substituent(s) may be present on any atom of the ring to which it is attached, where chemically feasible and valency rules permitting. For example, in the structure:

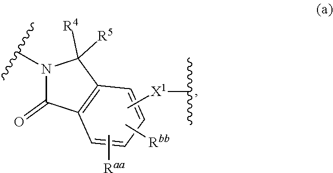

the R^{aa} substituent, and similarly the R^{bb} and X¹ substituents, can replace hydrogen of any CH that is part of the benzo portion of the bicyclic ring that is not already substituted with R^{bb} and X¹ (in the case of R^{aa}), and similarly with R^{aa} and X¹ (in the case of R^{bb}), and with R^{aa} and R^{bb} (in the case of X¹).

Additionally, as used throughout the application, including in the embodiments, when a group is drawn out as divalent, the left bond of the divalent group is attached to the group which is to its left in the remainder of the molecule, and the right bond of the divalent group is attached to the group which is to its right in the remainder of the molecule. For example, in Formula (Ia), in the following divalent groups:

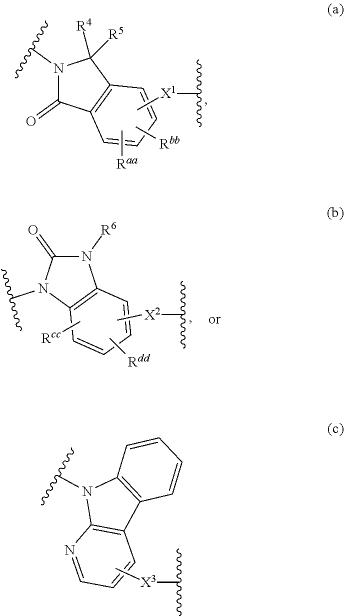

the [symbol] bond on the left of (a), (b) and (c) is attached to the following ring:

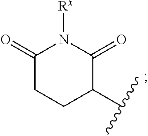

and the [symbol] on the right side of (a), (b), and (c) (i.e., X¹, X², and X³) is attached to Z¹ of L of the following structure:

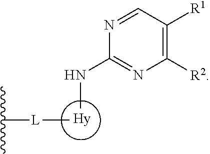

Similarly, for L i.e. —Z¹—Z²—Z³—Z⁴—Z⁵—Z⁶—, the left side in L (i.e., Z¹) is attached to X¹, X², X³, or X⁴ or point of attachment delineated in Degrons of formula (c), (d), (e), or (f) and Z⁶ is attached to an atom of Hy. For example, when L is a group of formula:

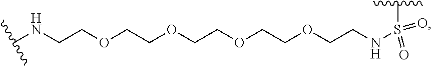 [SMILES: *NCCOCCOCCOCCOCCNS(*)(=O)=O]

and Degron is a group of formula (a), i.e.,

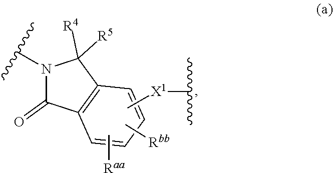

the left bond in L (i.e., the —NH— group) is attached to X¹ and the right hand bond in L (i.e., —SO₂—) is attached to an atom of the Hy

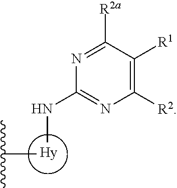

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a disease or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

“Treating” or “treatment” of a disease includes:
(1) preventing the disease, i.e., causing the clinical symptoms of the disease not to develop in a mammal that may be exposed to or predisposed to the disease but does not yet experience or display symptoms of the disease;
(2) inhibiting the disease, i.e., delaying, arresting, or reducing the development or severity of the disease or its clinical symptoms; or
(3) relieving the disease, i.e., causing regression of the disease or its clinical symptoms.

In one embodiment, treating or treatment of a disease includes inhibiting the disease, i.e., delaying, arresting or reducing the development or severity of the disease or its clinical symptoms; or relieving the disease, i.e., causing regression of the disease or its clinical symptoms.

A “therapeutically effective amount” means the amount of a compound of the present disclosure and/or a pharmaceutically acceptable salt thereof that, when administered to a patient for treating a disease, is sufficient to affect such treatment for the disease. The “therapeutically effective amount” will vary depending on the compound, the disease and its severity and the age, weight, etc., of the mammal to be treated.

A “condition associated with an autoimmune disease” means a condition that a patient with an autoimmune disease is susceptible to, e.g., sepsis, or a condition that is caused by the autoimmune disease, e.g., uveitis.

The compounds of the first aspect, second aspect, third aspect, and Formula (Ia) and embodiments thereof, can also inhibit CDK2. The term “inhibiting” and “reducing,” or any variation of these terms in relation of CDK2, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of CDK2 activity, compared to normal.

The term “degrading” and “degrade,” or any variation of these terms in relation of CDK2 and CDK1, means any measurable decrease in the concentration of CDK2 and CDK1, respectively, in a sample over time. For example, there may be a decrease of about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, in CDK2 concentration in a sample containing CDK2, and a compound disclosed herein in the Summary, Embodiments, and Compound Table 1 disclosed herein (including a compound of Formula (A1), (A), (I), or (Ia)) as compared to an equivalent sample comprising CDK2, in the absence of said compound. The % degradation can be determined as described in Biological Example 2 below. In one embodiment, the decrease in the concentration of CDK2≥40%. In one embodiment, the decrease in the concentration of CDK2≥50%. In one embodiment, the decrease in the concentration of CDK2≥60%. In one embodiment, the decrease in the concentration of CDK2≥70%. In one embodiment, the decrease in the concentration of CDK2≥80%.

“E3 ubiquitin ligase” refers to a family of proteins that operate in conjunction with E1 ubiquitin-activating enzyme and E2 ubiquitin-conjugating enzyme, assist or directly catalyze the covalent ligation of ubiquitin to a lysine residue of a substrate protein. E3 ubiquitin ligases directly bind to substrate proteins and thus confer substrate specificity for the ubiquitination process. Ubiquitination can serve as a versatile signal mark for substrate proteins, which are targeted to degradation by proteasome or other regulations ranging from translocation to transcription. The cereblon (CRBN) and von Hippel-Lindau (VHL) proteins are substrate recognition subunits of two ubiquitously expressed and biologically important Cullin RING E3 ubiquitin ligase complexes. Cereblon forms an E3 ubiquitin ligase complex with damaged DNA binding protein 1 (DDB1), Cullin-4A (CUL4A), and regulator of cullins 1 (ROC1). VHL is part of the E3 ligase complex VCB, which also consists of elongins B and C, Cul2 and Rbx1.

“E3 ubiquitin ligase ligand” means a small molecule ligand (i.e., having a molecular weight of below 2,000, 1,000, 500, or 200 Daltons), which is capable of binding to an E3 ubiquitin ligase or a subunit of E3 ligase, such as Cereblon, VHL, IAP, or MDM2.

### EMBODIMENTS

In embodiments A1A to A190, the present disclosure includes:
A1A. In embodiment A1A, provided is a compound of Formula (A1), or a pharmaceutically acceptable salt for use as described in the first aspect or an embodiment thereof as described in the Summary.
A1. In embodiment A1, provided is a compound of Formula (A), or a pharmaceutically acceptable salt for use as described in the second aspect of the Summary.
A2. In embodiment A2, provided is a compound of Formula (I) or a pharmaceutically acceptable salt for use as described in the third aspect of the Summary.
A3-1. In embodiment A3-1, the compound for use of embodiment A1A, A1, or A2, or a pharmaceutically acceptable salt thereof, is wherein the compound of Formula (A1), (A), or (1) is according to Formula (Ia1):

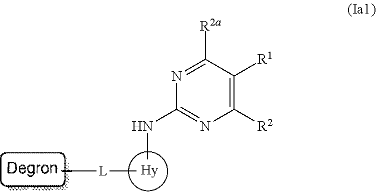

wherein:
R¹ is as defined in the first, second, or third aspect, respectively;
R² and R^{2a} are independently hydrogen or deuterium;
Hy is cycloalkylene, arylene, heteroarylene, heterocyclylene, bicyclic heterocyclylene, spiro heterocyclylene, bridged heterocyclylene, or fused heterocyclylene, where each of the aforementioned rings in this paragraph is substituted with R^{a}, R^{b}, and R^{c} independently selected from hydrogen, deuterium, alkyl, halo, haloalkyl, alkoxy, hydroxy, and cyano;
Degron is an E3 ubiquitin ligase ligand selected from:
(a) a group of formula (i):

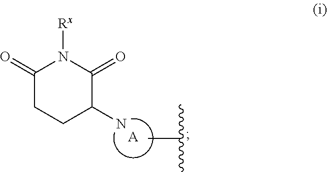

(b) a group of formula (ii):

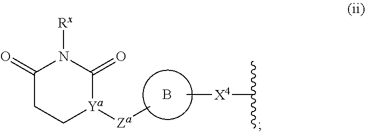

(c) a group of formula (iii):

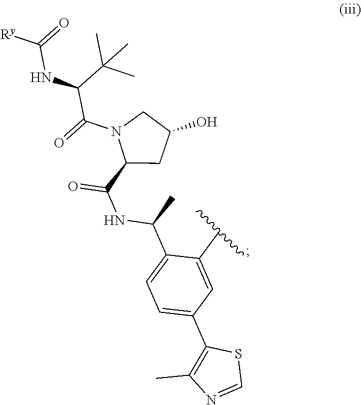

(d) a group of formula (iv):

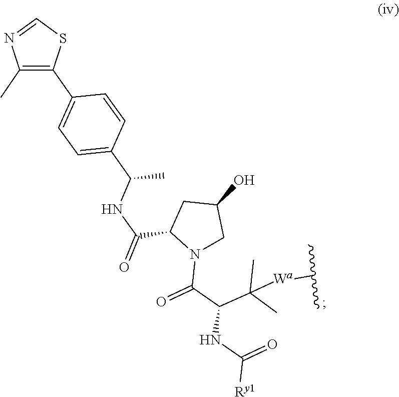

(e) a group of formula (v):

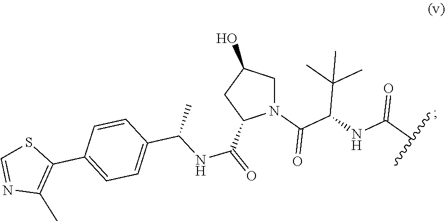 [SMILES: *C(=O)N[C@H](C(=O)N1C[C@H](O)C[C@H]1C(=O)N[C@@H](C)c1ccc(-c2scnc2C)cc1)C(C)(C)C]

and
(f) a group of formula (vi):

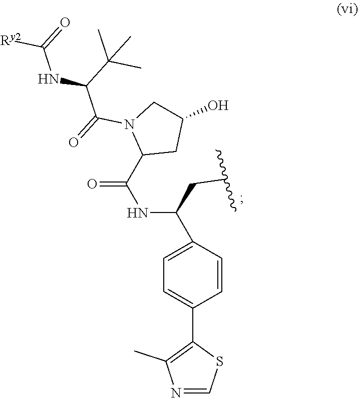

where:
   R^{x} and R^{x1} are each hydrogen;
   Y^{a} is CH or N;
   Z^{a} is a bond, —CH₂—, —NH—, —O—, or —NHC(O)— where NH of —NHC(O)— is attached to Y^{a};
   ring A is a group of formula (a), (b), or (c):

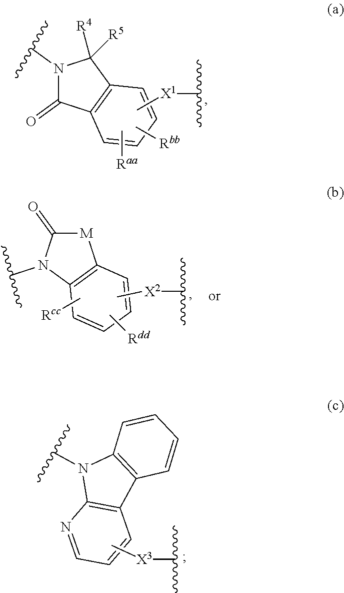

where:
   R^{aa}, R^{bb}, R^{cc}, and R^{dd} are independently selected from hydrogen, alkyl, alkoxy, halo, haloalkyl, haloalkoxy, and cyano;
   R⁴ and R⁵ are independently hydrogen or alkyl; or R⁴ and R⁵ together with the carbon to which they are attached form >C═O;
   M is —O— or —NR⁶—; and
   R⁶ is hydrogen or alkyl;
ring B is phenylene, cyclylaminylene, a 5- or 6-membered monocyclic heteroarylene, or a 9- or 10-membered fused bicyclic heteroarylene, wherein each heteroarylene ring contains one to three nitrogen ring atoms (and does not contain any additional heteroatoms) and further wherein the phenylene, cyclylaminylene, and each heteroarylene are independently substituted with R^{ee} and R^{ff} independently selected from hydrogen, alkyl, cycloalkyl, alkoxy, halo, haloalkyl, haloalkoxy, and cyano; and
X¹, X², X³, and X⁴ are independently a bond, -alkylene-, —O—, —(O-alkylene)-, -(alkylene-O)—, —(NR^{gg}-alkylene)-, -(alkylene-NR^{hh})—,

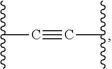 [SMILES: *C#C*]

—NH—, —N(alkyl)-, —C(═O)—, —NR^{jj}C(═O)—, or —C(═O)NR^{kk}— where R^{gg}, R^{hh}, R^{jj}, and R^{kk} are independently hydrogen, alkyl, or cycloalkyl and each alkylene is optionally substituted with one or two fluoro;
R^{y}, R^{y1}, and R^{y2} are independently alkyl, hydroxyalkyl, cycloalkyl or heterocyclyl wherein cycloalkyl and heterocyclyl are substituted with R^{d} and R^{f} selected from hydrogen, halo, cyano, alkylcarbonyl, and alkylcarbonylamino; and
W^{a} is bond, O, S, or alkylene; and
L is —Z¹—Z²—Z³—Z⁴—Z⁵—Z⁶— where:
Z¹ is a bond, alkylene, —C(O)NR—, —NR′(CO)—, —S(O)₂NR—, —NR′S(O)₂—, —(O-alkylene)ₐ-, -(alkylene-O)ₐ—, phenylene, monocyclic heteroarylene, or heterocyclylene, where each ring is substituted with R^{h} and Rⁱ independently selected from hydrogen, deuterium, alkyl, alkoxy, halo, haloalkyl, haloalkoxy, cyano, hydroxy, carboxy, alkoxycarbonyl, amino, alkylamino, and dialkylamino;
Z² is a bond, alkylene, alkynylene, —C(O)—, —C(O)N(R)—, —NR′(CO)—, —(O-alkylene)_{b}-, -(alkylene-O)_{b}—, —O(CH₂)₇—, —O(CH₂)₈—, cycloalkylene, or -heterocyclylene, where each ring is substituted with R^{j} and R^{k} independently selected from hydrogen, deuterium, alkyl, alkoxy, halo, haloalkyl, haloalkoxy, cyano, hydroxy, carboxy, alkoxycarbonyl, amino, alkylamino, and dialkylamino;
Z³ is a bond, alkylene, alkynylene, —C(O)NR—, —NR′(CO)—, —O—, —NR″—, —(O-alkylene)_{c}-, -(alkylene-O)_{c}—, cycloalkylene, spiro cyclolalkylene, phenylene, -(alkylene)-phenylene-, -phenylene-(alkylene)-, monocyclic heteroarylene, -(alkylene)-monocyclic heteroarylene-, -monocyclic heteroarylene-(alkylene)-, heterocyclylene, -(alkylene)-heterocyclylene-, -heterocyclylene-(alkylene)-, bicyclic heterocyclylene, bridged heterocyclylene, -(alkylene)-bridged heterocyclylene-, -bridged heterocyclylene-(alkylene)-, fused heterocyclylene, spiro heterocyclylene, -(alkylene)-spiro heterocyclylene-, -spiro heterocyclylene-(alkylene)-, or 11 to 13 membered spiro heterocyclylene, where each ring, by itself or as part of another group, is substituted with R^{m} and Rⁿ independently selected from hydrogen, deuterium, alkyl, alkoxy, halo, haloalkyl, haloalkoxy, cyano, hydroxy, carboxy, alkoxycarbonyl, amino, alkylamino, and dialkylamino;
Z⁴ is a bond, alkylene, alkynylene, -(alkylene-NR″)—, —(NR″-alkylene)-, —O—, —C(O)—, —NR″—, —(O)-alkylene)_{d}-, -(alkylene-O)_{d}—, cycloalkylene, -(alkylene)-cycloalkylene-, -cycloalkylene-(alkylene)-, spiro cyclolalkylene, phenylene, heteroarylene, heterocyclylene, -(alkylene)-heterocyclylene-, -heterocyclylene-(alkylene)-, fused heterocyclylene, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p} independently selected from hydrogen, deuterium, alkyl, alkoxy, halo, haloalkyl, haloalkoxy, cyano, hydroxy, carboxy, alkoxycarbonyl, amino, alkylamino, and dialkylamino;
Z⁵ is a bond, -alkylene, —NR″—, —O—, —C(O)—, —S(O)₂—, —NR′(CO)—, —C(O)NR—, phenylene, monocyclic heteroarylene, or heterocycylene, where each ring is substituted with R^{q} and R^{r} independently selected from hydrogen, deuterium, alkyl, alkoxy, halo, haloalkyl, haloalkoxy, cyano, hydroxy, carboxy, alkoxycarbonyl, amino, alkylamino, and dialkylamino; and
Z⁶ is a bond, alkylene, —NR″—, —O—, -(alkylene-O)—, —C(O)—, —S(O)₂—, —NR′(CO)—, or —C(O)NR—;

where each R, R′ and R″ is independently hydrogen or alkyl, each a, b, c, and d is independently an integer selected from 1 to 6, and each alkylene of —Z¹—, —Z²—, —Z³—, —Z⁴—, —Z⁵— and —Z⁶—, by itself or as part of another group, is independently substituted with R^{s} and R^{t} where R^{s} is hydrogen or deuterium and R^{t} is hydrogen, deuterium, haloalkyl, hydroxy, alkoxy, cyano, cycloalkyl, heterocyclyl, aryl, or monocyclic heteroaryl, wherein cycloalkyl, heterocyclyl, aryl, monocyclic heteroaryl are substituted with one or two substituents independently selected from hydrogen, alkyl, alkoxy, halo, haloalkyl, haloalkoxy, or cyano; provided that at least one of —Z¹—Z²—Z³—Z⁴—Z⁵—Z⁶— is not a bond; or
a pharmaceutically acceptable salt thereof.
A3. In embodiment A3, the compound for use of embodiment A1A, A1, A2, or A3-1, or a pharmaceutically acceptable salt thereof, is wherein the compound of Formula (IA1), (IA), (I), or (Ia1) respectively, is according to Formula (Ia):

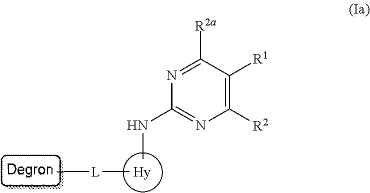

wherein R¹, R², R^{2a}, Hy, and L are as defined in embodiment A3-1; and
Degron is an E3 ubiquitin ligase ligand selected from:
   (a) a group of formula (i):

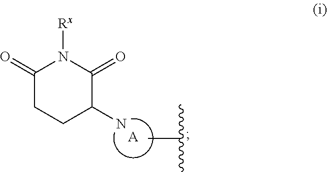

(b) a group of formula (ii):

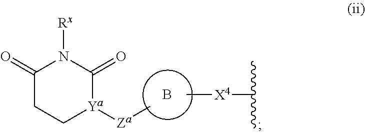

(c) a group of formula (iii):

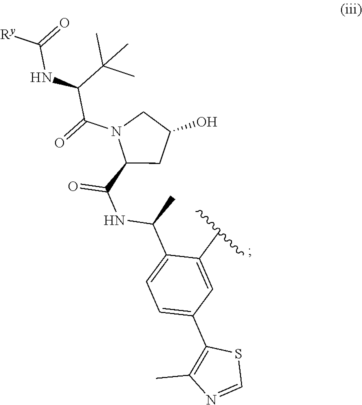

(d) a group of formula (iv):

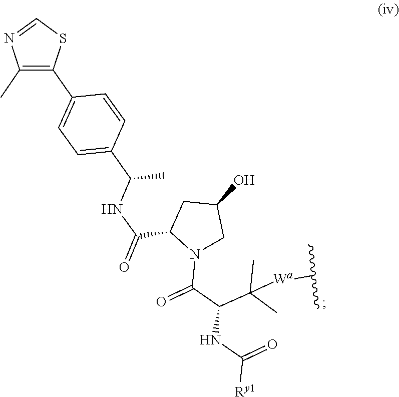

(e) a group of formula (v):

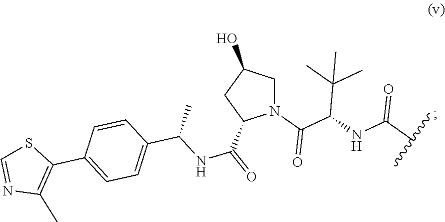 [SMILES: *C(=O)N[C@H](C(=O)N1C[C@H](O)C[C@H]1C(=O)N[C@@H](C)c1ccc(-c2scnc2C)cc1)C(C)(C)C]

and
(f) a group of formula (vi):

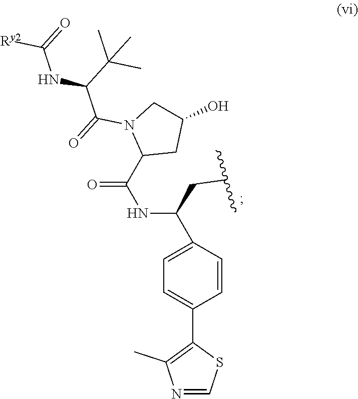

where:
   ring A is a group of formula (a), (b), or (c):

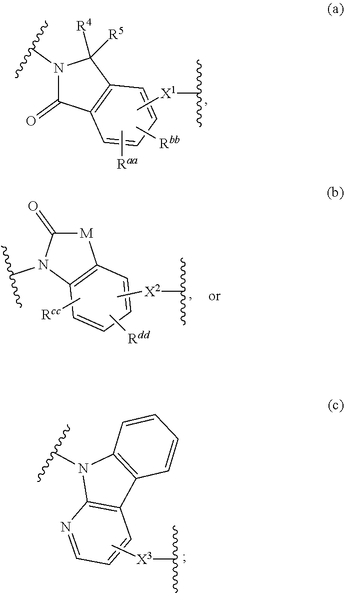

where:
   M is —NR⁶— where R⁶ is hydrogen or alkyl; and
R^{x}, R^{x1}, R^{y}, R^{y1}, R^{y2}, W^{a}, ring B, Y^{a}, Z^{a}, X¹, X², X³, X⁴, R⁴, R⁵, R^{aa}, R^{bb}, R^{cc}, and R^{dd} are as defined in A3-1.
A3A. In embodiment A3A, the compound for use of embodiment A3, or a pharmaceutically acceptable salt thereof, is wherein:
Z¹ is a bond, alkylene, —C(O)NR—, —NR′(CO)—, —S(O)₂NR—, —NR′S(O)₂—, —(O-alkylene)ₐ-, -(alkylene-O)ₐ—, phenylene, monocyclic heteroarylene, or heterocyclylene, where each ring is substituted with R^{h} and Rⁱ independently selected from hydrogen, deuterium, alkyl, alkoxy, halo, haloalkyl, haloalkoxy, cyano, hydroxy, amino, alkylamino, and dialkylamino;
Z² is a bond, alkylene, alkynylene, —C(O)—, —C(O)N(R)—, —NR′(CO)—, —(O-alkylene)_{b}-, -(alkylene-O)_{b}—, —O(CH₂)₇—, —O(CH₂)₈—, cycloalkylene, or -heterocyclylene, where each ring is substituted with R^{j} and R^{k} independently selected from hydrogen, deuterium, alkyl, alkoxy, halo, haloalkyl, haloalkoxy, cyano, hydroxy, amino, alkylamino, and dialkylamino;
Z³ is a bond, alkylene, alkynylene, —C(O)NR—, —NR′(CO)—, —O—, —NR″—, —(O-alkylene)_{c}-, -(alkylene-O)_{c}—, cycloalkylene, spiro cyclolalkylene, phenylene, -(alkylene)-phenylene-, -phenylene-(alkylene)-, monocyclic heteroarylene, -(alkylene)-monocyclic heteroarylene-, -monocyclic heteroarylene-(alkylene)-, heterocyclylene, -(alkylene)-heterocyclylene-, -heterocyclylene-(alkylene)-, bicyclic heterocyclylene, bridged heterocyclylene, -(alkylene)-bridged heterocyclylene-, -bridged heterocyclylene-(alkylene)-, fused heterocyclylene, spiro heterocyclylene, -(alkylene)-spiro heterocyclylene-, -spiro heterocyclylene-(alkylene)-, or 11 to 13 membered spiro heterocyclylene, where each ring, by itself or as part of another group, is substituted with R^{m} and Rⁿ independently selected from hydrogen, deuterium, alkyl, alkoxy, halo, haloalkyl, haloalkoxy, cyano, hydroxy, amino, alkylamino, and dialkylamino;
Z⁴ is a bond, alkylene, alkynylene, -(alkylene-NR″)—, —(NR″-alkylene)-, —O—, —C(O)—, —NR″—, —(O-alkylene)_{d}-, -(alkylene-O)_{d}—, cycloalkylene, -(alkylene)-cycloalkylene-, -cycloalkylene-(alkylene)-, spiro cyclolalkylene, phenylene, heteroarylene, heterocyclylene, -(alkylene)-heterocyclylene-, -heterocyclylene-(alkylene)-, fused heterocyclylene, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p} independently selected from hydrogen, deuterium, alkyl, alkoxy, halo, haloalkyl, haloalkoxy, cyano, hydroxy, amino, alkylamino, and dialkylamino;
Z⁵ is a bond, -alkylene, —NR″—, —O—, —C(O)—, —S(O)₂—, —NR′(CO)—, —C(O)NR—, phenylene, monocyclic heteroarylene, or heterocycylene, where each ring is substituted with R^{q} and R^{r} independently selected from hydrogen, deuterium, alkyl, alkoxy, halo, haloalkyl, haloalkoxy, cyano, hydroxy, amino, alkylamino, and dialkylamino; and
Z⁶ is a bond, alkylene, —NR″—, —O—, -(alkylene-O)—, —C(O)—, —S(O)₂—, —NR′(CO)—, or —C(O)NR—; or
a pharmaceutically acceptable salt thereof.
A4-1. In embodiment A4-1, the compound for use of embodiment A1A, A2, A3-1, A3, or A3A, or a pharmaceutically acceptable salt thereof, is wherein R¹ is alkylthio, pentafluorothio, haloalkylthio, amino, alkylamino, dialkylamino, cycloalkyl, cycloalkoxy, cycloalkylalkyl, bridged cycloalkyl, bridged cycloalkoxy, bridged cycloalkylalkyl, cyanoalkyl, cyanoalkoxy, alkoxyalkyl, aminoalkyl, aminoalkoxy, alkylaminoalkyl, dialkylaminoalkyl, alkylaminoalkoxy, dialkylaminoalkoxy, acyl, azidocarbonyl, alkoxycarbonyl, alkylcarbonylamino, aminocarbonyl, alkylaminocarbonyl, dialkylaminocarbonyl, aminosulfonyl, alkylaminosulfonyl, dialkylaminosulfonyl, substituted sulfonyl, substituted sulfinyl, substituted ureido, aryl, aralkyl, aryloxy, heteroaryl, heteroaralkyl, heteroaryloxy, heterocyclyl, heterocyclylalkyl, heterocyclyloxy, fused heterocyclyl, fused heterocyclyloxy, or fused heterocyclylalkyl, wherein cycloalkyl, by itself or as part of cycloalkoxy and cycloalkylalkyl, aryl, by itself or as part of aralkyl and aryloxy, heteroaryl, by itself or as part of heteroaralkyl and heteroaryloxy, heterocyclyl, by itself or as part of heterocyclylalkyl and heterocyclyloxy, bridged cycloalkyl, alone or as part of bridged cycloalkoxy and bridged cycloalkylalkyl, and fused heterocyclyl, by itself or as part of fused heterocyclylalkyl and fused heterocyclyloxy, are substituted with one, two, or three substituents independently selected from hydrogen, alkyl, alkoxy, halo, haloalkyl, haloalkoxy, and cyano.
A4-2. In embodiment A4-2, the compound for use of embodiment A1A, A2, A3-1, A3, or A3A, or a pharmaceutically acceptable salt thereof, is wherein R¹ is aryl, heteroaryl, heterocyclyl, cyanoalkyl, alkoxyalkyl, aminoalkyl, alkylaminoalkyl, dialkylaminoalkyl, heteroaryloxy, cyanoalkoxy, alkylthio, amino, alkylamino, dialkylamino, —SCF₃, —SF₅, fused heterocyclyl, bridged cycloalkyl, cycloalkylalkyl, heterocyclylalkyl, aralkyl, aminoalkoxy, alkoxycarbonyl, alkylcarbonylamino, acyl, azidocarbonyl, aminocarbonyl, alkylaminocarbonyl, dialkylaminocarbonyl, substituted ureido, aminosulfonyl, alkylaminosulfonyl, dialkylaminosulfonyl, and substituted sulfonyl, wherein aryl, alone and in arylalkyl, heteroaryl, alone and in heteroaryloxy, heterocyclyl, fused heterocyclyl, bridged cycloalkyl, alone and in cycloalkylalkyl, and heterocyclyl, alone and in heterocyclylalkyl, are substituted with one, two, or three substituents independently selected from hydrogen, alkyl, alkoxy, halo, haloalkyl, haloalkoxy, and cyano.

In a subembodiment of embodiment A4-2, R¹ is phenyl, pyrazolyl, imidazolyl, oxazolyl, thiazolyl, triazolyl, tetrahydrofuranyl, tetrahydropyranyl, pyrrolidinyl, piperidinyl, morpholinyl, thiomorpholinyl, piperazinyl, cyanomethyl, cyanoethyl, methoxymethyl, aminomethyl, methylaminomethyl, dimethylaminomethyl, 2,3-dihydrobenzofuranyl, benzodihydropyranyl, 1,4-benzodioxanyl, 2,3-dihydrofuro[3,2-c]pyridine, 2,3-dihydrofuro[2,3-c]pyridine, or 1,2,3,4-tetrahydroquinolinyl; wherein each of the rings is substituted with hydrogen, alkyl, alkoxy, halo, haloalkyl, haloalkoxy, or cyano.
A4-3. In embodiment A4-3, the compound for use of embodiment A1A, A2, A3-1, A3, or A3A, or a pharmaceutically acceptable salt thereof, is wherein R¹ is aryl, heteroaryl, heterocyclyl, cyanoalkyl, alkoxyalkyl, aminoalkyl, alkylaminoalkyl, dialkylaminoalkyl, heteroaryloxy, cyanoalkoxy, alkylthio, amino, alkylamino, dialkylamino, —SCF₃, or —SF₅; wherein each of the rings is substituted with hydrogen, alkyl, alkoxy, halo, haloalkyl, haloalkoxy, or cyano.

In a subembodiment of embodiment A4-3, R¹ is phenyl (substituted with hydrogen, alkyl, alkoxy, halo, cyano, haloalkyl, or haloalkoxy), pyrazolyl, imidazolyl, oxazolyl, thiazolyl, triazolyl (substituted with hydrogen or alkyl), tetrahydrofuranyl, tetrahydropyranyl, pyrrolidinyl, piperidinyl, morpholinyl, thiomorpholinyl, piperazinyl (substituted with hydrogen, alkyl, alkoxy, halo, haloalkyl, haloalkoxy, or cyano) cyanomethyl, cyanoethyl, methoxymethyl, aminomethyl, methylaminomethyl, or dimethylaminomethyl.
A4-4. In embodiment A4-4, the compound for use of embodiment A1A, A2, A3-1, A3, or A3A, or a pharmaceutically acceptable salt thereof, is wherein R¹ is alkylthio (such as methylthio).
A4-5. In embodiment A4-5, the compound for use of embodiment A1A, A2, A3-1, A3, or A3A, or a pharmaceutically acceptable salt thereof, is wherein R¹ is pentafluorothio.
A4-6. In embodiment A4-6, the compound for use of embodiment A1A, A2, A3-1, A3, or A3A, or a pharmaceutically acceptable salt thereof, is wherein R¹ is haloalkylthio (such as trifluoromethylthio).
A4-7. In embodiment A4-7, the compound for use of embodiment A1A, A2, A3-1, A3, or A3A, or a pharmaceutically acceptable salt thereof, is wherein R¹ is amino.
A4-8. In embodiment A4-8, the compound for use of embodiment A1A, A2, A3-1, A3, or A3A, or a pharmaceutically acceptable salt thereof, is wherein R¹ is alkylamino (such as methylamino).
A4-9. In embodiment A4-9, the compound for use of embodiment A1A, A2, A3-1, A3, or A3A, or a pharmaceutically acceptable salt thereof, is wherein R¹ is dialkylamino (such as dimethylamino).
A4-10. In embodiment A4-10, the compound for use of embodiment A1A, A2, A3-1, A3, or A3A, or a pharmaceutically acceptable salt thereof, is wherein R¹ is cycloalkyl substituted with one, two, or three substituents independently selected from hydrogen, alkyl, alkoxy, halo, haloalkyl, haloalkoxy, and cyano. In a subembodiment of embodiment A4-10, the cycloalkyl is cyclopropyl, cyclobutyl, or cyclopentyl, each ring substituted with one or two substituents independently selected from hydrogen, methyl, fluoro, and cyano.
A4-11. In embodiment A4-11, the compound for use of embodiment A1A, A2, A3-1, A3, or A3A, or a pharmaceutically acceptable salt thereof, is wherein R¹ is cycloalkoxy where the cycloalkyl is substituted with one, two, or three substituents independently selected from hydrogen, alkyl, alkoxy, halo, haloalkyl, haloalkoxy, and cyano. In a subembodiment of embodiment A4-11, cycloalkyloxy is cyclopropyloxy, cyclobutyloxy, or cyclopentyloxy, each cycloalkyl ring of cycloalkyloxy substituted with one or two substituents independently selected from hydrogen, methyl, fluoro, and cyano.
A4-12. In embodiment A4-12, the compound for use of embodiment A1A, A2, A3-1, A3, or A3A, or a pharmaceutically acceptable salt thereof, is wherein R¹ is cycloalkylalkyl where the cycloalkyl is substituted with one, two, or three substituents independently selected from hydrogen, alkyl, alkoxy, halo, haloalkyl, haloalkoxy, and cyano. In a subembodiment of embodiment A4-12, cycloalkylalkyl is cyclopropylmethyl, cyclobutylmethyl, or cyclopentylmethyl, each ring of cycloalkylalkyl substituted with one or two substituents independently selected from hydrogen, methyl, fluoro, and cyano.
A4-13. In embodiment A4-13, the compound for use of embodiment A1A, A2, A3-1, A3, or A3A, or a pharmaceutically acceptable salt thereof, is wherein R¹ is bridged cycloalkyl (such as bicyclo[1.1.1]pent-1-yl or bicyclo[2.2.1]heptyl) where the bridged cycloalkyl is substituted with one, two, or three substituents independently selected from hydrogen, alkyl, alkoxy, halo, haloalkyl, haloalkoxy, and cyano. In a subembodiment of embodiment A4-13, the bridged cycloalkyl is substituted with one or two substituents independently selected from hydrogen, methyl, fluoro, and cyano.
A4-14. In embodiment A4-14, the compound for use of embodiment A1A, A2, A3-1, A3, or A3A, or a pharmaceutically acceptable salt thereof, is wherein R¹ is bridged cycloalkoxy (such as bicyclo[1.1.1]pent-1-yloxy or bicyclo[2.2.1]heptyloxy) where the bridged cycloalkyl of bridged cycloalkoxy is substituted with one, two, or three substituents independently selected from hydrogen, alkyl, alkoxy, halo, haloalkyl, haloalkoxy, and cyano. In a subembodiment of embodiment A4-14, the bridged cycloalkyloxy is substituted with one or two substituents independently selected from hydrogen, methyl, fluoro, and cyano.
A4-15. In embodiment A4-15, the compound for use of embodiment A1A, A2, A3-1, A3, or A3A, or a pharmaceutically acceptable salt thereof, is wherein R¹ is bridged cycloalkylalkyl (such as bicyclo[1.1.1]pent-1-ylmethyl or bicyclo[2.2.1]heptylmethyl) where the bridged cycloalkyl of bridged cyclylalkylalkyl is substituted with one, two, or three substituents independently selected from hydrogen, alkyl, alkoxy, halo, haloalkyl, haloalkoxy, and cyano. In a subembodiment of embodiment A4-15, the bridged cycloalkylalkyl is substituted with one or two substituents independently selected from hydrogen, methyl, fluoro, and cyano.
A4-16. In embodiment A4-16, the compound for use of embodiment A1A, A2, A3-1, A3, or A3A, or a pharmaceutically acceptable salt thereof, is wherein R¹ is cyanoalkyl. In a subembodiment of embodiment A4-16, R¹ is cyanomethyl or cyanoethyl,
A4-17. In embodiment A4-17, the compound for use of embodiment A1A, A2, A3-1, A3, or A3A, or a pharmaceutically acceptable salt thereof, is wherein R¹ is cyanoalkoxy (such as cyanomethoxy or cyanoethoxy).
A4-18. In embodiment A4-18, the compound for use of embodiment A1A, A2, A3-1, A3, or A3A, or a pharmaceutically acceptable salt thereof, is wherein R¹ is alkoxyalkyl. In a subembodiment of embodiment A4-18, R¹ is methoxymethyl.
A4-19. In embodiment A4-19, the compound for use of embodiment A1A, A2, A3-1, A3, or A3A, or a pharmaceutically acceptable salt thereof, is wherein R¹ is aminoalkyl. In a subembodiment of embodiment A4-19, R¹ is aminomethyl.
A4-20. In embodiment A4-20, the compound for use of embodiment A1A, A2, A3-1, A3, or A3A, or a pharmaceutically acceptable salt thereof, is wherein R¹ is aminoalkoxy. In a subembodiment of embodiment A4-20, R¹ is aminomethyloxy.
A4-21. In embodiment A4-21, the compound for use of embodiment A1A, A2, A3-1, A3, or A3A, or a pharmaceutically acceptable salt thereof, is wherein R¹ is alkylaminoalkyl. In a subembodiment of embodiment A4-21, R¹ is methylaminomethyl.
A4-22. In embodiment A4-22, the compound for use of embodiment A1A, A2, A3-1, A3, or A3A, or a pharmaceutically acceptable salt thereof, is wherein R¹ is dialkylaminoalkyl. In a subembodiment of embodiment A4-22, R¹ is dimethylaminomethyl.
A4-23. In embodiment A4-23, the compound for use of embodiment A1A, A2, A3-1, A3, or A3A, or a pharmaceutically acceptable salt thereof, is wherein R¹ is alkylaminoalkoxy. In a subembodiment of embodiment A4-23, R¹ is methylaminomethyloxy.
A4-24. In embodiment A4-24, the compound for use of embodiment A1A, A2, A3-1, A3, or A3A, or a pharmaceutically acceptable salt thereof, is wherein R¹ is dialkylaminoalkoxy. In a subembodiment of embodiment A4-24, R¹ is dimethylaminomethyloxy.
A4-25. In embodiment A4-25 the compound for use of embodiment A1A, A2, A3-1, A3, or A3A, or a pharmaceutically acceptable salt thereof, is wherein R¹ is acyl. In a subembodiment of embodiment A4-25, acyl is alkylcarbonyl (such as methylcarbonyl).
A4-26. In embodiment A4-26, the compound for use of embodiment A1A, A2, A3-1, A3, or A3A, or a pharmaceutically acceptable salt thereof, is wherein R¹ is azidocarbonyl.
A4-27. In embodiment A4-27, the compound for use of embodiment A1A, A2, A3-1, A3, or A3A, or a pharmaceutically acceptable salt thereof, is wherein R¹ is alkoxycarbonyl. In a subembodiment of embodiment A4-27, R¹ is methoxycarbonyl or ethoxycarbonyl.
A4-28. In embodiment A4-28, the compound for use of embodiment A1A, A2, A3-1, A3, or A3A, or a pharmaceutically acceptable salt thereof, is wherein R¹ is alkylcarbonylamino.
A4-29. In embodiment A4-29, the compound for use of embodiment A1A, A2, A3-1, A3, or A3A, or a pharmaceutically acceptable salt thereof, is wherein R¹ is aminocarbonyl.
A4-30. In embodiment A4-30, the compound for use of embodiment A1A, A2, A3-1, A3, or A3A, or a pharmaceutically acceptable salt thereof, is wherein R¹ is alkylaminocarbonyl (such as methylaminocarbonyl).
A4-31. In embodiment A4-31, the compound for use of embodiment A1A, A2, A3-1, A3, or A3A, or a pharmaceutically acceptable salt thereof, is wherein R¹ is dialkylaminocarbonyl (such as dimethylaminocarbonyl).
A4-32. In embodiment A4-32, the compound for use of embodiment A1A, A2, A3-1, A3, or A3A, or a pharmaceutically acceptable salt thereof, is wherein R¹ is aminosulfonyl.
A4-33. In embodiment A4-33, the compound for use of embodiment A1A, A2, A3-1, A3, or A3A, or a pharmaceutically acceptable salt thereof, is wherein R¹ is alkylaminosulfonyl (such as methylaminosulfonyl).
A4-34. In embodiment A4-34, the compound for use of embodiment A1A, A2, A3-1, A3, or A3A, or a pharmaceutically acceptable salt thereof, is wherein R¹ is dialkylaminosulfonyl (such as dimethylaminosulfonyl).
A4-35. In embodiment A4-35, the compound for use of embodiment A1A, A2, A3-1, A3, or A3A, or a pharmaceutically acceptable salt thereof, is wherein R¹ is substituted sulfonyl.
A4-36. In embodiment A4-36, the compound for use of embodiment A1A, A2, A3-1, A3, or A3A, or a pharmaceutically acceptable salt thereof, is wherein R¹ is substituted sulfinyl.
A4-37. In embodiment A4-37, the compound for use of embodiment ALA, A2, A3-1, A3, or A3A, or a pharmaceutically acceptable salt thereof, is wherein R¹ is substituted ureido.
A4-38. In embodiment A4-38, the compound for use of embodiment A1A, A2, A3-1, A3, or A3A, or a pharmaceutically acceptable salt thereof, is wherein R¹ is aryl substituted with one, two, or three substituents independently selected from hydrogen, alkyl, alkoxy, halo, haloalkyl, haloalkoxy, and cyano. In a first embodiment of subembodiment A4-38, R¹ is phenyl substituted with one, two, or three substituents independently selected from hydrogen, alkyl, alkoxy, halo, haloalkyl, haloalkoxy, and cyano. In a second subembodiment of embodiment A4-38, R¹ is phenyl substituted with one, two, or three substituents independently selected from hydrogen, methyl, fluoro, cyano, difluoromethyl, trifluoromethyl, difluoromethoxy and trifluoromethoxy.
A4-39. In embodiment A4-39, the compound for use of embodiment A1A, A2, A3-1, A3, or A3A, or a pharmaceutically acceptable salt thereof, is wherein R¹ is aralkyl (such as benzyl) where the aryl is substituted with one, two, or three substituents independently selected from hydrogen, alkyl, alkoxy, halo, haloalkyl, haloalkoxy, and cyano. In a subembodiment of embodiment A4-39, R¹ is benzyl where phenyl of benzyl is substituted with one, two, or three substituents independently selected from hydrogen, methyl, fluoro, cyano, difluoromethyl, trifluoromethyl, difluoromethoxy and trifluoromethoxy.
A4-40. In embodiment A4-40, the compound for use of embodiment A1A, A2, A3-1, A3, or A3A, or a pharmaceutically acceptable salt thereof, is wherein R¹ is heteroaryl where the heteroaryl is substituted with one, two, or three substituents independently selected from hydrogen, alkyl, alkoxy, halo, haloalkyl, haloalkoxy, and cyano. In a first embodiment of subembodiment A4-40, R¹ is pyrazolyl, imidazolyl, oxazolyl, thiazolyl, or triazolyl, each of which is substituted with one, two, or three substituents independently selected from hydrogen, alkyl, alkoxy, halo, haloalkyl, haloalkoxy, and cyano. In a second subembodiment of embodiment A4-40, R¹ is pyrazolyl, imidazolyl, oxazolyl, thiazolyl, or triazolyl, each of which is substituted with one substituent selected from hydrogen and alkyl.
A4-41. In embodiment A4-41, the compound for use of embodiment A1A, A2, A3-1, A3, or A3A, or a pharmaceutically acceptable salt thereof, is wherein R¹ is heteroaralkyl where the heteroaryl of heteroaralkyl is substituted with one, two, or three substituents independently selected from hydrogen, alkyl, alkoxy, halo, haloalkyl, haloalkoxy, and cyano. In a subembodiment of embodiment A4-41, the heteroaryl of heteroaralkyl of R¹ is pyrazolyl, imidazolyl, oxazolyl, thiazolyl, or triazolyl, each of which is substituted with one, two, or three substituents independently selected from hydrogen, alkyl, alkoxy, halo, haloalkyl, haloalkoxy, and cyano.
A4-42. In embodiment A4-42, the compound for use of embodiment A1A, A2, A3-1, A3, or A3A, or a pharmaceutically acceptable salt thereof, is wherein R¹ is heteroaryloxy where the heteroaryl is substituted with one, two, or three substituents independently selected from hydrogen, alkyl, alkoxy, halo, haloalkyl, haloalkoxy, and cyano. In a subembodiment of embodiment A4-42, the heteroaryl of heteroaryloxy of R¹ is pyrazolyl, imidazolyl, oxazolyl, thiazolyl, or triazolyl, each of which is substituted with one, two, or three substituents independently selected from hydrogen, alkyl, alkoxy, halo, haloalkyl, haloalkoxy, and cyano.
A4-43. In embodiment A4-43, the compound for use of embodiment A1A, A2, A3-1, A3, or A3A, or a pharmaceutically acceptable salt thereof, is wherein R¹ is heterocyclyl where the heterocyclyl is substituted with one, two, or three substituents independently selected from hydrogen, alkyl, alkoxy, halo, haloalkyl, haloalkoxy, and cyano. In a subembodiment of embodiment A4-43, R¹ is tetrahydrofuranyl, tetrahydropyranyl, pyrrolidinyl, piperidinyl, morpholinyl, thiomorpholinyl, or piperazinyl where the piperazinyl is optionally substituted with alkyl, alkoxy, halo, haloalkyl, haloalkoxy, or cyano.
A4-44. In embodiment A4-44, the compound for use of embodiment A1A, A2, A3-1, A3, or A3A, or a pharmaceutically acceptable salt thereof, is wherein R¹ is heterocyclylalkyl where the heterocyclyl is substituted with one, two, or three substituents independently selected from hydrogen, alkyl, alkoxy, halo, haloalkyl, haloalkoxy, and cyano. In a subembodiment of embodiment A4-44, the heterocyclyl of heterocyclylalkyl of R¹ is tetrahydrofuranyl, tetrahydropyranyl, pyrrolidinyl, piperidinyl, morpholinyl, thiomorpholinyl, or piperazinyl where the piperazinyl is optionally substituted with alkyl, alkoxy, halo, haloalkyl, haloalkoxy, or cyano.
A4-45. In embodiment A4-45, the compound for use of embodiment A1A, A2, A3-1, A3, or A3A, or a pharmaceutically acceptable salt thereof, is wherein R¹ is heterocyclyloxy where the heterocyclyl is substituted with one, two, or three substituents independently selected from hydrogen, alkyl, alkoxy, halo, haloalkyl, haloalkoxy, and cyano. In a subembodiment of embodiment A4-45, the heterocyclyl of heterocyclyloxy of R¹ is tetrahydrofuranyl, tetrahydropyranyl, pyrrolidinyl, piperidinyl, morpholinyl, thiomorpholinyl, or piperazinyl where the piperazinyl is optionally substituted with alkyl, alkoxy, halo, haloalkyl, haloalkoxy, or cyano.
A4-46. In embodiment A4-46, the compound for use of embodiment A1A, A2, A3-1, A3, or A3A, or a pharmaceutically acceptable salt thereof, is wherein R¹ is fused heterocyclyl substituted with one, two, or three substituents independently selected from hydrogen, alkyl, alkoxy, halo, haloalkyl, haloalkoxy, and cyano. In a subembodiment of embodiment A4-6, R¹ is 2,3-dihydrobenzofuranyl, benzodihydropyranyl, 1,4-benzodioxanyl, 2,3-dihydrofuro[3,2-c]pyridine, 2,3-dihydrofuro[2,3-c]pyridine, or 1,2,3,4-tetrahydroquinolinyl, each of which is substituted with one, two, or three substituents independently selected from hydrogen, alkyl, alkoxy, halo, haloalkyl, haloalkoxy, and cyano.
A4-47. In embodiment A4-47, the compound for use of embodiment A1A, A2, A3-1, A3, or A3A, or a pharmaceutically acceptable salt thereof, is wherein R¹ is fused heterocyclyloxy where the heterocyclyl is substituted with one, two, or three substituents independently selected from hydrogen, alkyl, alkoxy, halo, haloalkyl, haloalkoxy, and cyano.
A4-48. In embodiment A4-48, the compound for use of embodiment A1A, A2, A3-1, A3, or A3A, or a pharmaceutically acceptable salt thereof, is wherein R¹ is fused heterocyclylalkyl where the heterocyclyl is substituted with one, two, or three substituents independently selected from hydrogen, alkyl, alkoxy, halo, haloalkyl, haloalkoxy, and cyano.
A4-49. In embodiment A4-49, the compound for use of embodiment A1A, A2, A3-1, A3, A3A, or A4-1 to A4-48, or a pharmaceutically acceptable salt thereof, is wherein R¹ is selected from:

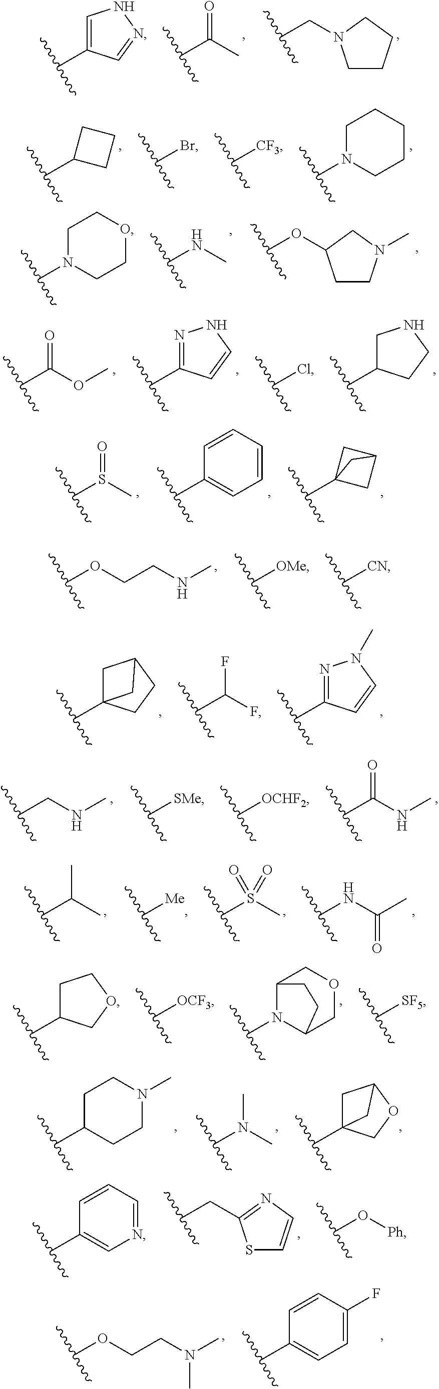

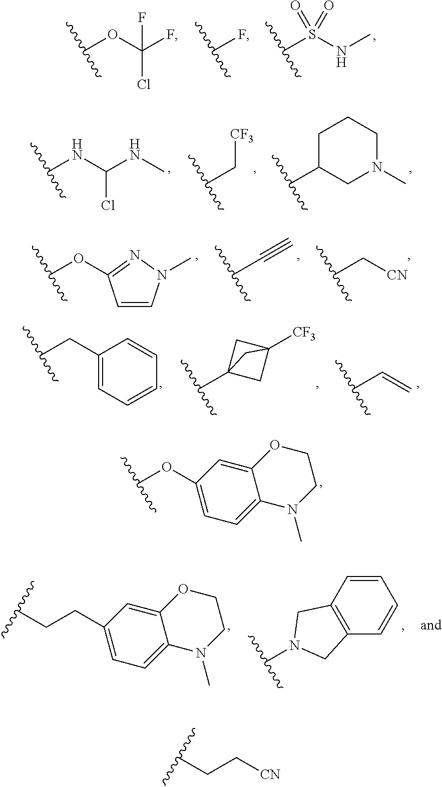

and isomers (R and/or S isomers, and/or geometric isomers) thereof.
A4-50. In embodiment A4-50, the compound for use of embodiment A1A, A1, A2, A3-1, A3, or A3A, or a pharmaceutically acceptable salt thereof, is wherein R¹ is alkyl, alkenyl, alkynyl, cycloalkyl, halo, haloalkyl, haloalkoxy, alkoxy, aryloxy, cyano, or cycloalkyl where the cycloalkyl is substituted with one to three halo.
A4. In embodiment A4, the compound for use of embodiment A1A, A1, A2, A3-1, A3, A3A, or A4-50, or a pharmaceutically acceptable salt thereof, is wherein R¹ is halo, haloalkyl, or haloalkoxy.
A5. In embodiment A5, the compound for use of any one of embodiments A1 to A3A, A4-50, and A4, or a pharmaceutically acceptable salt thereof, is wherein R¹ is halo.
A6. In embodiment A6, the compound for use of any one of embodiments A1 to A3A, A4-50, and A4, or a pharmaceutically acceptable salt thereof, is wherein R¹ is haloalkyl.
A7. In embodiment A7, the compound for use of any one of embodiments A1 to A3A, A4-50, and A4, or a pharmaceutically acceptable salt thereof, is wherein R¹ is haloalkoxy.
A8. In embodiment A8, the compound for use of any one of embodiments A1 to A3A, A4-50, and A4 to A7, or a pharmaceutically acceptable salt thereof, is wherein R¹ is chloro, bromo, fluoro, difluoromethyl, trifluoromethyl, difluoroethyl, trifluoroethyl, difluoromethoxy, trifluoromethoxy, difluoroethoxy, or trifluoroethoxy.
A9. In embodiment A9, the compound for use of any one of embodiments A1 to A3A, A4-50, and A4 to A8, or a pharmaceutically acceptable salt thereof, is wherein R¹ is chloro, bromo, difluoromethyl, trifluoromethyl, difluoromethoxy, or trifluoromethoxy.
A10. In embodiment A10, the compound for use of any one of embodiments A1 to A3A, A4-50, A4, A5, A8, and A9, or a pharmaceutically acceptable salt thereof, is wherein R¹ is chloro or bromo.

A11. In embodiment A11, the compound for use of any one of embodiments A1 to A3A, A4-50, A4, A6, A8, and A9, or a pharmaceutically acceptable salt thereof, is wherein R¹ is difluoromethyl or trifluoromethyl.
A12. In embodiment A12, the compound for use of any one of embodiments A1 to A3A, A4-50, A4, A6, A8, A9, and A11, or a pharmaceutically acceptable salt thereof, is wherein R¹ is trifluoromethyl.
A13. In embodiment A13, the compound for use of embodiment A1, A2, A3-1, A3, A3A, or A4-50, or a pharmaceutically acceptable salt thereof, is wherein R¹ is alkyl, alkenyl, or alkynyl.
A14. In embodiment A14, the compound for use of embodiment A1, A2, A3-1, A3, A3A, A4-50, or A13, or a pharmaceutically acceptable salt thereof, is wherein R¹ is methyl, ethyl, propyl, vinyl, propenyl, ethynyl, or propynyl.
A15. In embodiment A15, the compound for use of embodiment A1, A2, A3-1, A3, A3A, A13, A4-50, or A14, or a pharmaceutically acceptable salt thereof, is wherein R¹ is methyl, ethyl, or propyl.
A16. In embodiment A16, the compound for use of embodiment A1, A2, A3-1, A3, A3A, A13, A4-50, or A14, or a pharmaceutically acceptable salt thereof, is wherein R¹ is vinyl, propenyl, ethynyl, or propynyl.
A17. In embodiment A17, the compound for use of embodiment A1, A2, A3-1, A3, A3A, or A4-50, or a pharmaceutically acceptable salt thereof, is wherein R¹ is alkoxy.
A18. In embodiment A18, the compound for use of embodiment A1, A2, A3-1, A3, A3A, A4-50, or A17, or a pharmaceutically acceptable salt thereof, is wherein R¹ is methoxy, ethoxy, or propoxy.
A19. In embodiment A19, the compound for use of embodiment A1, A2, A3-1, A3, A3A, or A4-50, or a pharmaceutically acceptable salt thereof, is wherein R¹ is aryloxy (such as phenoxy).
A20. In embodiment A20, the compound for use of embodiment A1, A2, A3-1, A3, A3A, or A4-50, or a pharmaceutically acceptable salt thereof, is wherein R¹ is cyano.
A21. In embodiment A21, the compound for use of embodiment A1, A2, A3-1, A3, A3A, or A4-50, or a pharmaceutically acceptable salt thereof, is wherein R¹ is cycloalkyl (such as cyclopropyl).
A22. In embodiment A22, the compound for use of embodiment A1, A2, A3-1, A3, A3A, or A4-50, or a pharmaceutically acceptable salt thereof, is wherein R¹ is cycloalkyl substituted with one to three halo (such as fluorocyclopropyl or difluorocyclopropyl).
A23. In embodiment A23, the compound for use of any one of embodiments A1A and A1 to A22, or a pharmaceutically acceptable salt thereof, is wherein R² and R^{2a} are hydrogen.
A24. In embodiment A24, the compound for use of any one of embodiments A1A and A1 to A22, or a pharmaceutically acceptable salt thereof, is wherein one of R² and R^{2a} is deuterium and the other of R² and R^{2a} is hydrogen or both R² and R^{2a} are deuterium.
A25. In embodiment A25, the compound for use of any one of embodiments A1A and A1 to A24, or a pharmaceutically acceptable salt thereof, is wherein Hy is heterocyclylene, phenylene, spiro heterocyclylene, bridged heterocyclylene, or cycloalkylene, wherein each of the aforementioned rings is substituted with R^{a}, R^{b}, and R^{c} where R^{a} and R^{b} are independently selected from hydrogen, deuterium, alkyl, halo, haloalkyl, alkoxy, hydroxy, and cyano, and R^{c} is hydrogen.
A26. In embodiment A26, the compound for use of any one of embodiments A1A and A1 to A25, or a pharmaceutically acceptable salt thereof, is wherein Hy is heterocyclylene substituted with R^{a}, R^{b}, and R^{c} where R^{a} and R^{b} are independently selected from hydrogen, deuterium, alkyl, halo, haloalkyl, alkoxy, hydroxy, and cyano, and R^{c} is hydrogen.
A27. In embodiment A27, the compound for use of any one of embodiments A1A and A1 to A26, or a pharmaceutically acceptable salt thereof, is wherein the heterocyclylene of Hy is pyrrolidin-1,3-diyl or piperidin-1,4-diyl, where Hy is substituted with R^{a}, R^{b}, and R^{c} where R^{a} and R^{b} are independently hydrogen, deuterium, methyl, fluoro, methoxy, or hydroxy, R^{c} is hydrogen, and L is attached to the nitrogen atom of the piperidin-1,4-diyl or pyrrolidin-1,3-diyl ring of Hy.
A28. In embodiment A28, the compound for use of any one of embodiments A1A and A1 to A27, or a pharmaceutically acceptable salt thereof, is wherein the heterocyclylene of Hy is:

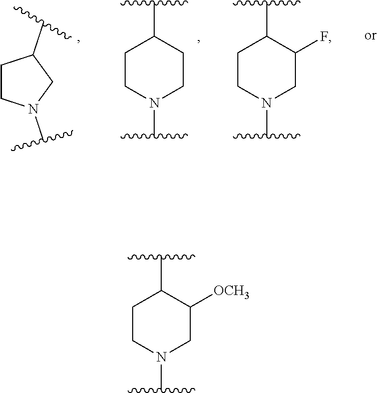 [SMILES: *C1CCN(*)C1 ; *C1CCN(*)CC1 ; *C1CCN(*)CC1F ; "or" ; *C1CCN(*)CC1OC]

where the N atom of the pyrrolidin-1,3-diyl or piperidin-1,4-diyl rings is attached to L.
A29. In embodiment A29, the compound for use of any one of embodiments A1A and A1 to A28, or a pharmaceutically acceptable salt thereof, is wherein the heterocyclylene of Hy is:

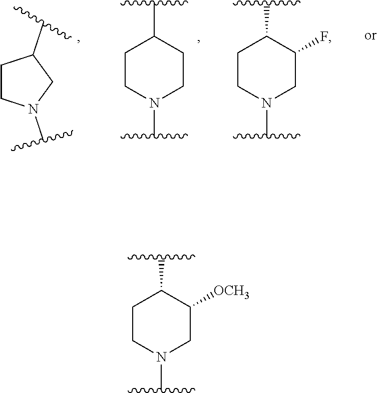 [SMILES: *C1CCN(*)C1 ; *C1CCN(*)CC1 ; *[C@H]1CCN(*)C[C@H]1F ; "or" ; *[C@H]1CCN(*)C[C@H]1OC]

where the N atom of the pyrrolidin-1,3-diyl or piperidin-1,4-diyl rings is attached to L.
A29a. In embodiment A29a, the compound for use of any one of embodiments A1A and A1 to A29, or a pharmaceutically acceptable salt thereof, is wherein the heterocyclylene of Hy is:

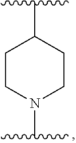 [SMILES: *C1CCN(*)CC1]

where the N atom of the piperidin-1,4-diyl ring is attached to L.
A30. In embodiment A30, the compound for use of any one of embodiments A1A and A1 to A25, or a pharmaceutically acceptable salt thereof, is wherein Hy is bridged heterocyclylene substituted with R^{a}, R^{b}, and R^{c} independently selected from hydrogen, deuterium, alkyl, halo, haloalkyl, alkoxy, hydroxy, and cyano.
A31. In embodiment A31, the compound for use of any one of embodiments A1A, A1 to A25, and A30, or a pharmaceutically acceptable salt thereof, is wherein the bridged heterocyclylene of Hy is a ring of formula:

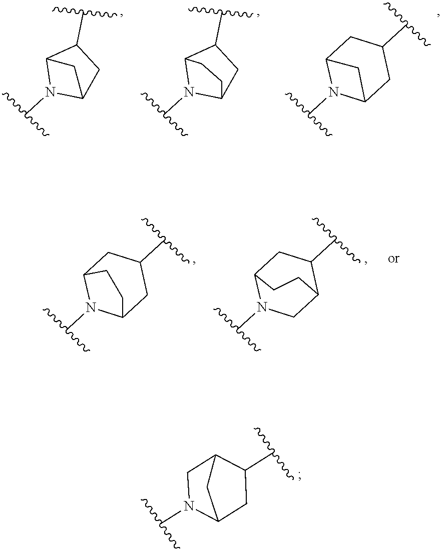 [SMILES: *C1CC2CC1N2* ; *C1CC2CCC1N2* ; *C1CC2CC(C1)N2* ; *C1CC2CCC(C1)N2* ; *C1CC2CCC1CN2* ; *C1CC2CC1CN2*]

and each ring is substituted with R^{a}, R^{b}, and R^{c} where R^{c} is hydrogen, and L is attached to the nitrogen atom of each ring.
A32. In embodiment A32, the compound for use of embodiment A30 or A31, or a pharmaceutically acceptable salt thereof, is wherein R^{a} and R^{b} are independently hydrogen, deuterium, methyl, fluoro, methoxy, or hydroxy.
A33. In embodiment A33, the compound for use of embodiment A30, A31, or A32, or a pharmaceutically acceptable salt thereof, is wherein R^{b} is hydrogen.
A34. In embodiment A34, the compound for use of any one of embodiments A1A and A1 to A25, or a pharmaceutically acceptable salt thereof, is wherein Hy is cycloalkylene substituted with R^{a}, R^{b}, and R^{c} where R^{a} is deuterium, methyl, fluoro, methoxy, or hydroxy and R^{b} and R^{c} are hydrogen.
A35. In embodiment A35, the compound for use of any one of embodiments A1A, A1 to A25, and A34, or a pharmaceutically acceptable salt thereof, is wherein the cycloalkylene of Hy is cyclohexylene.
A36. In embodiment A36, the compound for use of any one of embodiments A1A, A1 to A25, A34, and A35, or a pharmaceutically acceptable salt thereof, is wherein the cycloalkylene of Hy is

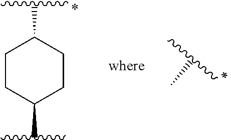

denotes bond to NH and [symbol] denotes bond of L.
A37. In embodiment A37, the compound for use of any one of embodiments A1A and A1 to A25, or a pharmaceutically acceptable salt thereof, is wherein Hy is arylene wherein the arylene is phenylene substituted with R^{a}, R^{b}, and R^{c} where R^{a} and R^{b} are independently selected from hydrogen, deuterium, alkyl, halo, haloalkyl, alkoxy, hydroxy, and cyano, and R^{c} is hydrogen.
A38. In embodiment A38, the compound for use of any one of embodiments A1A and A1 to A25, or a pharmaceutically acceptable salt thereof, is wherein Hy is spiro heterocyclylene substituted (such as 2-azaspiro[3.3]heptan-2-yl) with R^{a}, R^{b}, and R^{c} where R^{a} and R^{b} are independently selected from hydrogen, deuterium, alkyl, halo, haloalkyl, alkoxy, hydroxy, and cyano, and R^{c} is hydrogen.
A39. In embodiment A39, the compound for use of any one of embodiments A1A, A1 to A25, and A37, or a pharmaceutically acceptable salt thereof, is wherein the phenylene of Hy is 1,4-phenylene according to structure

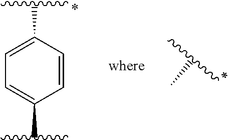

denotes bond to NH and [symbol] denotes bond of L.
A39a. In embodiment A39a, the compound for use of any one of embodiments A1A and A1 to A24, or a pharmaceutically acceptable salt thereof, is wherein Hy is fused heterocyclylene substituted with R^{a}, R^{b}, and R^{c} where R^{a} and R^{b} are independently selected from hydrogen, deuterium, alkyl, halo, haloalkyl, alkoxy, hydroxy, and cyano, and R^{c} is hydrogen.
A39b. In embodiment A39b, the compound for use of any one of embodiments A1A and A1 to A24, or a pharmaceutically acceptable salt thereof, is wherein Hy is bicyclic heterocyclylene substituted with R^{a}, R^{b}, and R^{c} where R^{a} and R^{b} are independently selected from hydrogen, deuterium, alkyl, halo, haloalkyl, alkoxy, hydroxy, and cyano, and R^{c} is hydrogen.
A40A. In embodiment A40A, the compound for use of any one of embodiments A2 to A39b, or a pharmaceutically acceptable salt thereof, is wherein the Degron is an E3 ubiquitin ligase ligand of formula (i) or (ii) as disclosed in embodiment A3-1.
A40. In embodiment A40, the compound for use of any one of embodiments A2, A3-1, A3 to A39b, and A40A, or a pharmaceutically acceptable salt thereof, is wherein the Degron is an E3 ubiquitin ligase ligand of formula (i):

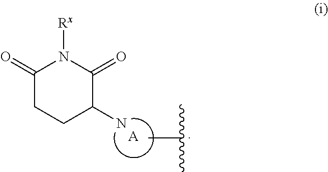

A41. In embodiment A41, the compound for use of any one of embodiments A3-1, A3 to A40, or a pharmaceutically acceptable salt thereof, is wherein the ring A of the E3 ubiquitin ligase ligand of formula (i) is a group of formula (a):

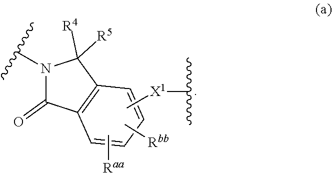

A42. In embodiment A42, the compound for use of any one of embodiments A3-1, A3 to A41, or a pharmaceutically acceptable salt thereof, is wherein R⁴ and R⁵ are independently hydrogen or alkyl.
A43. In embodiment A43, the compound for use of any one of embodiments A3-1, A3 to A42, or a pharmaceutically acceptable salt thereof, is wherein R⁴ and R⁵ are hydrogen.
A44. In embodiment A44, the compound for use of any one of embodiments A3-1, A3 to A42, or a pharmaceutically acceptable salt thereof, is wherein R⁴ is hydrogen and R⁵ is methyl.
A45. In embodiment A45, the compound for use of any one of embodiments A3-1 and A3 to A41, or a pharmaceutically acceptable salt thereof, is wherein R⁴ and R⁵ together with the carbon to which they are attached form >C═O.
A46. In embodiment A46, the compound for use of any one of embodiments A3-1 and A3 to A40, or a pharmaceutically acceptable salt thereof, is wherein the ring A of the E3 ubiquitin ligase ligand of formula (i) is a group of formula (b):

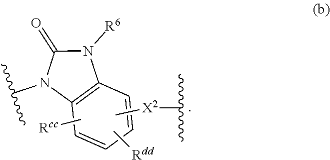

A47. In embodiment A47, the compound for use of any one of embodiments A3-1, A3 to A40, and A46, or a pharmaceutically acceptable salt thereof, is wherein R⁶ is hydrogen.
A48. In embodiment A48, the compound for use of any one of embodiments A3-1, A3 to A40, and A46, or a pharmaceutically acceptable salt thereof, wherein R⁶ is alkyl, preferably methyl.
A49. In embodiment A49, the compound for use of any one of embodiments A3-1 and A3 to A40, or a pharmaceutically acceptable salt thereof, is wherein the ring A of the E3 ubiquitin ligase ligand of formula (i) is a group of formula (c):

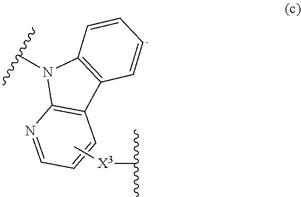

A50A. In embodiment A50A, the compound for use of any one of embodiments A3-1, and A4-1 to A40, or a pharmaceutically acceptable salt thereof, is wherein the ring A of the E3 ubiquitin ligase ligand of formula (i) is:

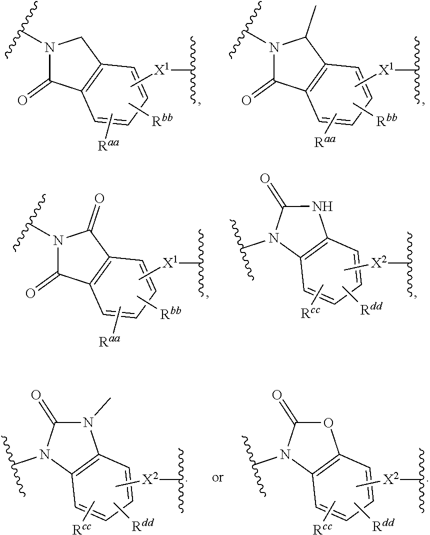

A50. In embodiment A50, the compound for use of any one of embodiments A3 to A40 and A50A, or a pharmaceutically acceptable salt thereof, is wherein the ring A of the E3 ubiquitin ligase ligand of formula (i) is:

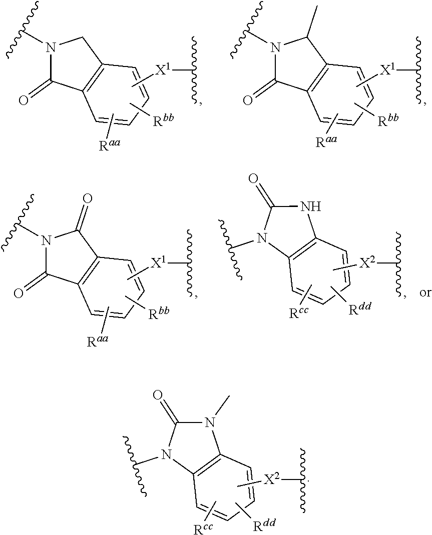

A51A. In embodiment A51A, the compound for use of any one of embodiments A3-1, A4-1 to A40, and A50A, or a pharmaceutically acceptable salt thereof, is wherein ring A of the E3 ubiquitin ligase ligand of formula (i) is:

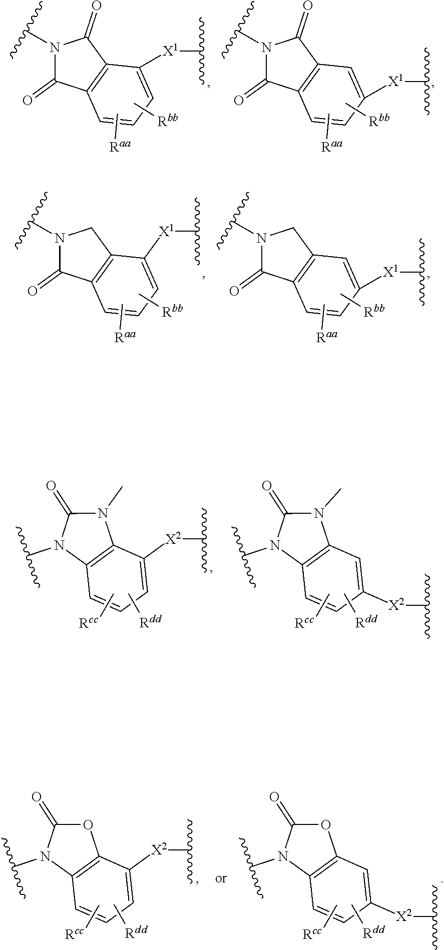

A51. In embodiment A51, the compound for use of any one of embodiments A3 to A40 and A50, or a pharmaceutically acceptable salt thereof, is wherein ring A of the E3 ubiquitin ligase ligand of formula (i) is:

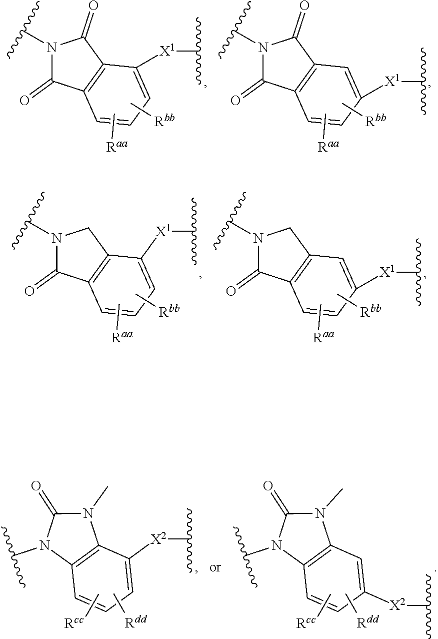

A52A. In embodiment A52A, the compound for use of any one of embodiments A3-1, A4-1 to A40, A50A, and A51A, or a pharmaceutically acceptable salt thereof, is wherein ring A of the E3 ubiquitin ligase ligand of formula (i) is:

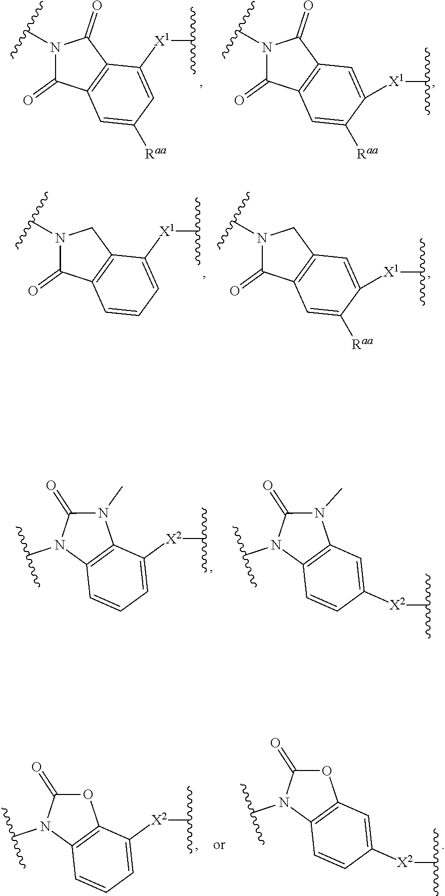

A52. In embodiment A52, the compound for use of any one of embodiments A3 to A40, A50, and A51, or a pharmaceutically acceptable salt thereof, is wherein ring A of the E3 ubiquitin ligase ligand of formula (i) is:

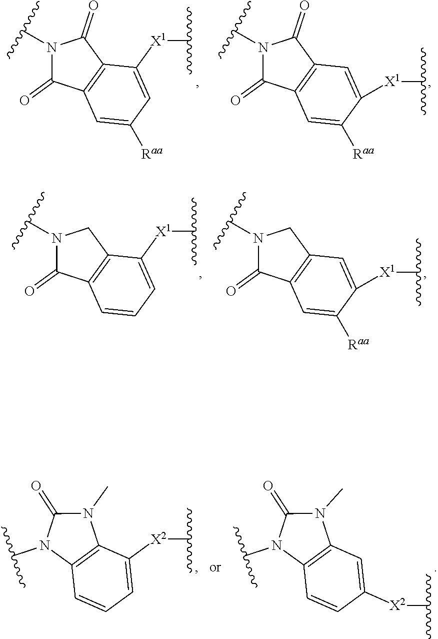

A52a. In embodiment A52a, the compound for use of any one of embodiments A3-1 to A41, A45, and A50A to A52, or a pharmaceutically acceptable salt thereof, is wherein ring A of the E3 ubiquitin ligase ligand of formula (i) is:

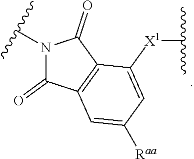

A53. In embodiment A53, the compound for use of any one of embodiments A3-1 to A41, A45, and A50A to A52, or a pharmaceutically acceptable salt thereof, is wherein ring A of the E3 ubiquitin ligase ligand of formula (i) is:

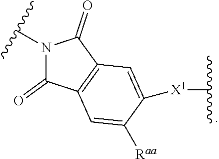

A54. In embodiment A54, the compound for use of any one of embodiments A3-1 to A43, and A50A to A52, or a pharmaceutically acceptable salt thereof, is wherein ring A of the E3 ubiquitin ligase ligand of formula (i) is:

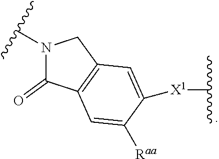

A55. In embodiment A55, the compound for use of any one of embodiments A3-1 to A43, and A50A to A52, or a pharmaceutically acceptable salt thereof, is wherein ring A of the E3 ubiquitin ligase ligand of formula (i) is:

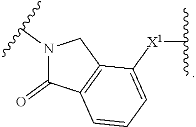

A56. In embodiment A56, the compound for use of any one of embodiments A3-1 to A40, A46, A48, and A50A to A52, or a pharmaceutically acceptable salt thereof, is wherein ring A of the E3 ubiquitin ligase ligand of formula (i) is:

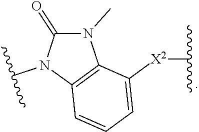

A57. In embodiment A57, the compound for use of any one of embodiments A3-1 to A40, A46, A48, and A50A to A52, or a pharmaceutically acceptable salt thereof, is wherein ring A of the E3 ubiquitin ligase ligand of formula (i) is:

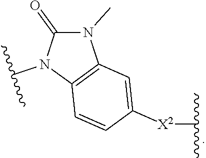

A58. In embodiment A58, the compound for use of any one of embodiments A3-1 to A48 and A50A to A54, or a pharmaceutically acceptable salt thereof, is wherein R^{aa}, R^{bb}, R^{cc}, and R^{dd} are independently selected from hydrogen, alkyl, alkoxy, halo, haloalkyl, and haloalkoxy. For sake of clarity, R^{aa}, R^{bb}, R^{cc}, and/or R^{dd} are hydrogen when they are not specifically drawn out in structures of formula (i) and (ii), respectively.
A59. In embodiment A59, the compound for use of any one of embodiments A3-1 to A48 and A50A to A54, or a pharmaceutically acceptable salt thereof, is wherein R^{aa}, R^{bb}, R^{cc}, and R^{dd} are independently selected from hydrogen, alkyl, alkoxy, halo, haloalkyl, and cyano.
A60. In embodiment A60, the compound for use of any one of embodiments A3-1 to A48, A50A to A54, A58, and A59, or a pharmaceutically acceptable salt thereof, is wherein R^{aa}, R^{bb}, R^{cc}, and R^{dd} are independently selected from hydrogen, methyl, methoxy, ethoxy, fluoro, trifluoromethyl, difluoromethyl, and trifluoromethoxy.
A61. In embodiment A61, the compound for use of any one of embodiments A3-1 to A48, A50A to A54, and A58 to A60, or a pharmaceutically acceptable salt thereof, is wherein R^{aa}, R^{bb}, R^{cc}, and R^{dd} are independently selected from hydrogen and methyl.
A62. In embodiment A62, the compound for use of any one of embodiments A3-1 to A48, A50A to A54, and A58 to A60, or a pharmaceutically acceptable salt thereof, is wherein R^{aa}, R^{bb}, R^{cc}, and R^{dd} are independently selected from hydrogen and methoxy.
A63. In embodiment A63, the compound for use of any one of embodiments A3-1 to A48, A50A to A54, and A58 to A60, or a pharmaceutically acceptable salt thereof, is wherein R^{aa}, R^{bb}, R^{cc}, and R^{dd} are independently selected from hydrogen and fluoro.
A64. In embodiment A64, the compound for use of any one of embodiments A3-1 to A48, A50A to A54, and A58 to A60, or a pharmaceutically acceptable salt thereof, is wherein R^{aa}, R^{bb}, R^{cc}, and R^{dd} are independently selected from hydrogen, trifluoromethyl, and difluoromethyl.
A65. In embodiment A65, the compound for use of any one of embodiments A3-1 to A48, A50A to A54, A58, and A60, or a pharmaceutically acceptable salt thereof, is wherein R^{aa}, R^{bb}, R^{cc}, and R^{dd} are independently selected from hydrogen and trifluoromethoxy.
A66. In embodiment A66, the compound for use of any one of embodiments A3-1 to A48, A50A to A54, and A58 to A60, or a pharmaceutically acceptable salt thereof, is wherein R^{aa}, R^{bb}, R^{cc}, and R^{dd} are independently selected from hydrogen, fluoro, and trifluoromethyl.
A67. In embodiment A67, the compound for use of any one of embodiments A2, A3-1 to A39b, and A40A, or a pharmaceutically acceptable salt thereof, is wherein the Degron is an E3 ubiquitin ligase ligand of formula (ii):

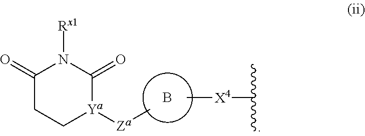

A68. In embodiment A68, the compound for use of any one of embodiments A3-1 to A39b, A40A, and A67, or a pharmaceutically acceptable salt thereof, is wherein Y^{a} is CH.
A69. In embodiment A69, the compound for use of any one of embodiments A3-1 to A39b, A40A, and A67, or a pharmaceutically acceptable salt thereof, is wherein Y^{a} is N.
A70. In embodiment A70, the compound for use of any one of embodiments A3-1 to A39b, A40A, and A67 to A69, or a pharmaceutically acceptable salt thereof, is wherein Z^{a} is a bond, —NH—, —O—, or —NHC(O)—.
A71. In embodiment A71, the compound for use of any one of embodiments A3-1 to A39b, A40A, and A67 to A70, or a pharmaceutically acceptable salt thereof, is wherein Z^{a} is a bond, —NH—, or —NHC(O)—.
A72. In embodiment A72, the compound for use of any one of embodiments A3-1 to A39b, A40A, and A67 to A71, or a pharmaceutically acceptable salt thereof, is wherein Z^{a} is a bond.
A73. In embodiment A73, the compound for use of any one of embodiments A3-1 to A39b, A40A, and A67 to A71, or a pharmaceutically acceptable salt thereof, is wherein Z^{a} is —NH—, or —NHC(O)—.
A74. In embodiment A74, the compound for use of any one of embodiments A3-1 to A39b, A40A, A67 to A71, and A73, or a pharmaceutically acceptable salt thereof, is wherein Z^{a} is —NH—.
A74a. In embodiment A74a, the compound for use of any one of embodiments A3-1 to A39b, A40A, A67 to A71, and A73, or a pharmaceutically acceptable salt thereof, is wherein Z^{a} is —NHC(O)—.
A75. In embodiment A75, the compound for use of any one of embodiments A3-1 to A39b, A40A, and A67 to A74a, or a pharmaceutically acceptable salt thereof, is wherein ring B is phenylene substituted with R^{ee} and R^{ff}.
A76. In embodiment A76, the compound for use of any one of embodiments A3-1 to A39b, A40A, and A67 to A74a, or a pharmaceutically acceptable salt thereof, is wherein ring B is cyclylaminylene substituted with R^{ee} and R^{ff}.
A77. In embodiment A77, the compound for use of any one of embodiments A3-1 to A39b, A40A, and A67 to A74a, or a pharmaceutically acceptable salt thereof, is wherein ring B is 5- or 6-membered monocyclic heteroarylene or a 9- or 10-membered fused bicyclic heteroarylene, wherein each heteroarylene ring contains one to three nitrogen ring atoms and each ring is substituted with R^{ee} and R^{ff}
A78. In embodiment A78, the compound for use of any one of embodiments A3-1 to A39b, A40A, A67 to A74a, and A77, or a pharmaceutically acceptable salt thereof, is wherein ring B is 5- or 6-membered monocyclic heteroarylene containing one or two nitrogen ring atoms substituted with R^{ee} and R^{ff}.
A79. In embodiment A79, the compound for use of any one of embodiments A3-1 to A39b, A40A, A67 to A74a, and A77, or a pharmaceutically acceptable salt thereof, is wherein ring B is a 9- or 10-membered fused bicyclic heteroarylene containing one to three nitrogen ring atoms (and not containing any additional heteroatoms) and substituted with R^{ee} and R^{ff}.
A80. In embodiment A80, the compound for use of any one of embodiments A3-1 to A39b, A40A, A67 to A74a, A77, and A79, or a pharmaceutically acceptable salt thereof, is wherein ring B is a 9- or 10-membered fused bicyclic heteroarylene containing one or two nitrogen ring atoms and substituted with R^{ee} and R^{ff}.
A81. In embodiment A81, the compound for use of any one of embodiments A3-1 to A39b, A40A, and A67 to A80, or a pharmaceutically acceptable salt thereof, is wherein the E3 ubiquitin ligase ligand of formula (ii) is:

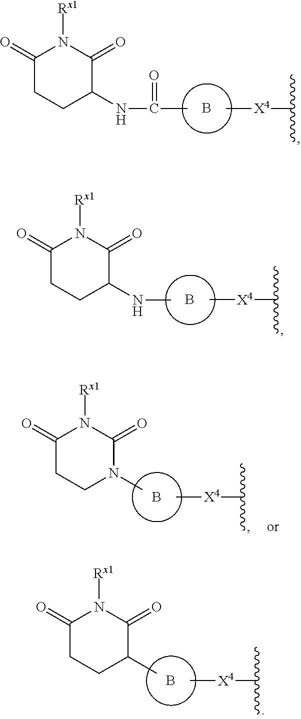

A82-1. In embodiment A82-1, the compound for use of any one of embodiments A3-1 to A39b, A40A, and A67 to A81, or a pharmaceutically acceptable salt thereof, is wherein the E3 ubiquitin ligase ligand of formula (ii) is:

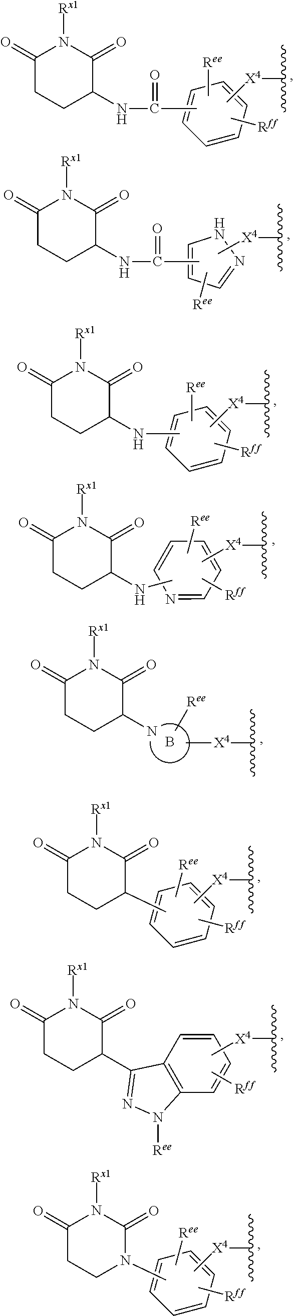

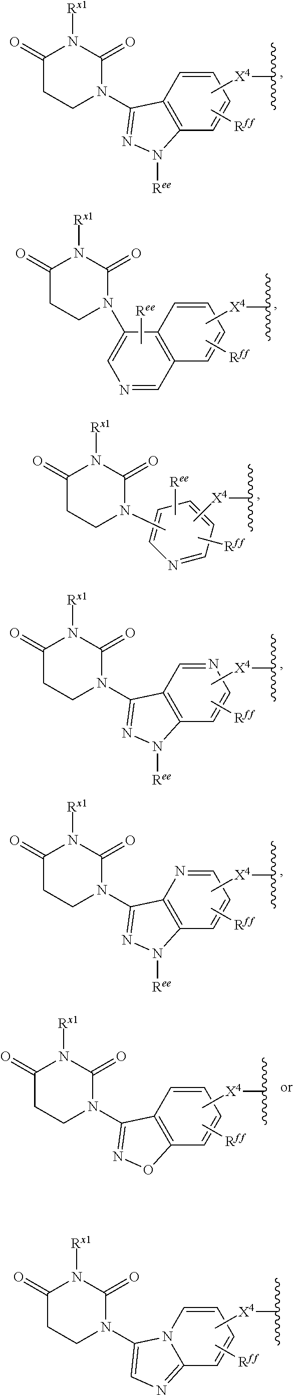

where ring B is cyclylaminylene.
A82. In embodiment A82, the compound for use of any one of embodiments A3 to A39b, A40A, and A67 to A82-1, or a pharmaceutically acceptable salt thereof, is wherein the E3 ubiquitin ligase ligand of formula (ii) is:

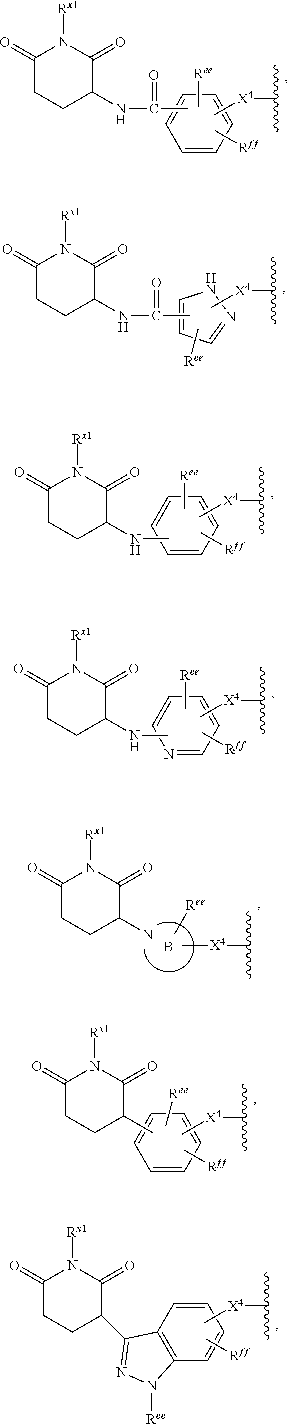

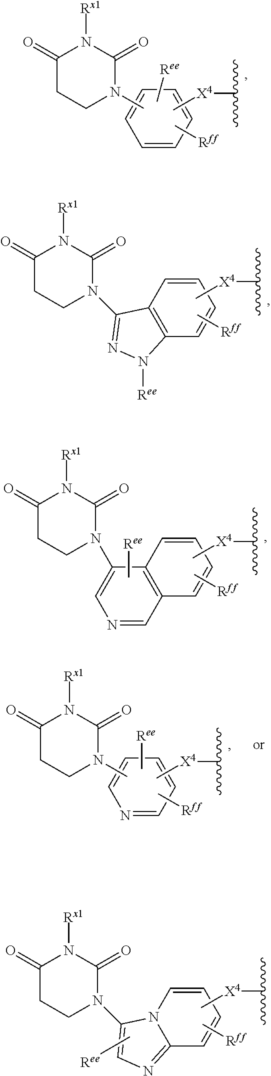

where ring B is cyclylaminylene.
A82A. In embodiment A82A, the compound for use of any one of embodiments A3 to A39b, A40A, A67, A68, A70 to A72, A77, and A79 to A82-1, or a pharmaceutically acceptable salt thereof, is wherein the E3 ubiquitin ligase ligand of formula (ii) is

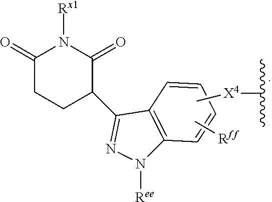

A83. In embodiment A83, the compound for use of any one of embodiments A3 to A39b, A40A, and A67 to A82, or a pharmaceutically acceptable salt thereof, is wherein the E3 ubiquitin ligase ligand of formula (ii) is:

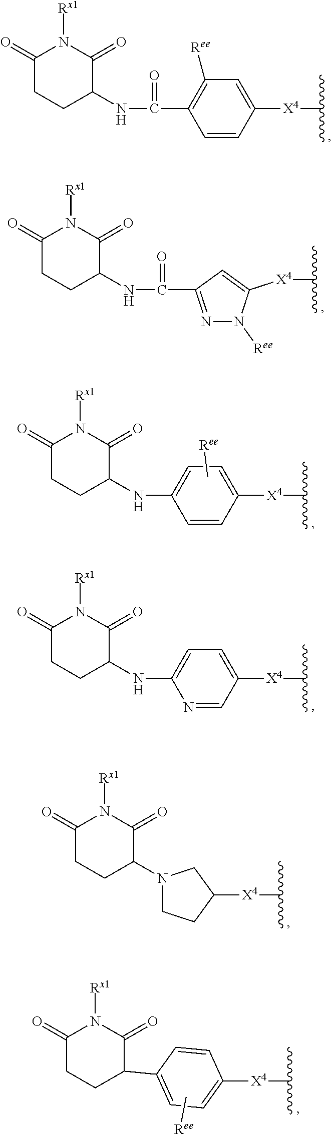

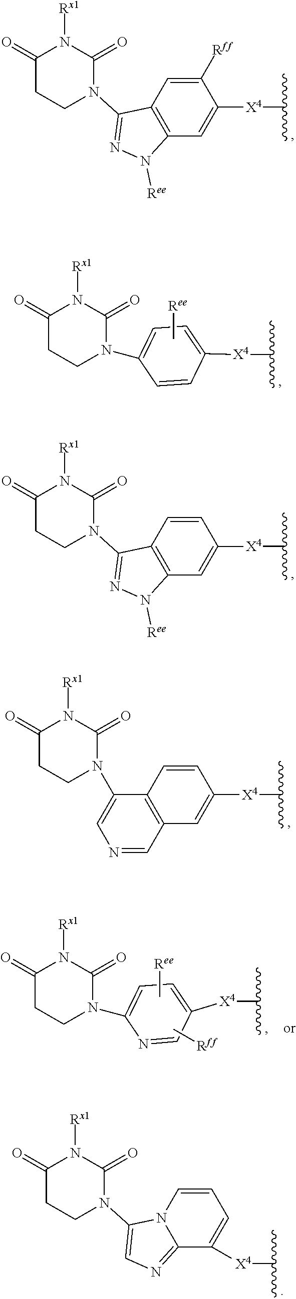

A83A. In embodiment A83A, the compound for use of any one of embodiments A3 to A39b, A40A, and A67, A69 to A72, A77, A79 to A82, and A83, or a pharmaceutically acceptable salt thereof, is wherein the E3 ubiquitin ligase ligand of formula (ii) is

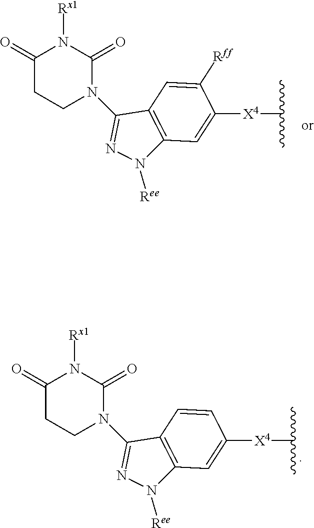

A84. In embodiment A84, the compound for use of any one of embodiments A3-1 to A39b, A40A, and A67 to A83A, or a pharmaceutically acceptable salt thereof, is wherein each R^{ee} and R^{ff} are independently selected from hydrogen, alkyl, alkoxy, halo, cyano, haloalkyl, and haloalkoxy.
A85. In embodiment A85, the compound for use of any one of embodiments A3-1 to A39b, A40A, and A67 to A83A, or a pharmaceutically acceptable salt thereof, is wherein R^{ee} and R^{ff} are independently selected from hydrogen, alkyl, cycloalkyl, alkoxy, halo, haloalkyl, and cyano.
A86. In embodiment A86, the compound for use of any one of embodiments A3-1 to A39b, A40A, and A67 to A85, or a pharmaceutically acceptable salt thereof, is wherein R^{ee} and R^{ff} are independently selected from hydrogen, methyl, ethyl, isopropyl, cyclopropyl, methoxy, ethoxy, fluoro, chloro, trifluoromethyl, 2,2,2-trifluoroethyl, difluoromethyl, difluoromethoxy, trifluoromethoxy, and cyano.
A87. In embodiment A87, the compound for use of any one of embodiments A3-1 to A39b, A40A, and A67 to A86, or a pharmaceutically acceptable salt thereof, is wherein R^{ee} and R^{ff} are independently selected from hydrogen, methyl, ethyl, and isopropyl.
A88. In embodiment A88, the compound for use of any one of embodiments A3-1 to A39b, A40A, and A67 to A86, or a pharmaceutically acceptable salt thereof, is wherein R^{ee} and R^{ff} are independently selected from hydrogen and methoxy.
A89. In embodiment A89, the compound for use of any one of embodiments A3-1 to A39b, A40A, and A67 to A86, or a pharmaceutically acceptable salt thereof, is wherein R^{ee} and R^{ff} are independently selected from hydrogen, methyl, ethyl, isopropyl, chloro, and fluoro.
A90. In embodiment A90, the compound for use of any one of embodiments A3-1 to A39b, A40A, and A67 to A86, or a pharmaceutically acceptable salt thereof, is wherein one of R^{ee} and R^{ff} is hydrogen or fluoro and the other of R^{ee} and R^{ff} is selected from hydrogen, trifluoromethyl, 2,2,2-trifluoroethyl, and difluoromethyl.
A91. In embodiment A91, the compound for use of any one of embodiments A3-1 to A39b, A40A, and A67 to A86, or a pharmaceutically acceptable salt thereof, is wherein R^{ee} and R^{ff} are independently selected from hydrogen, difluoromethoxy, and trifluoromethoxy.
A92. In embodiment A92, the compound for use of any one of embodiments A3-1 to A39b, A40A, and A67 to A86, or a pharmaceutically acceptable salt thereof, is wherein R^{ee} and R^{ff} are independently selected from hydrogen, chloro, fluoro, and trifluoromethyl.
A93. In embodiment A93, the compound for use of any one of embodiments A3-1 to A39b, A40A, and A67 to A86, or a pharmaceutically acceptable salt thereof, is wherein R^{ee} and R^{ff} are hydrogen.
A94. In embodiment A94, the compound for use of any one of embodiments A3-1 to A39b, A40A, and A67 to A86, or a pharmaceutically acceptable salt thereof, is wherein R^{ee} and R^{ff} are chloro.
A95. In embodiment A95, the compound for use of any one of embodiments A3-1 to A39b, A40A, and A67 to A86, or a pharmaceutically acceptable salt thereof, is wherein R^{ee} and R^{ff} are fluoro.
A96. In embodiment A96, the compound for use of any one of embodiments A3-1 to A39b, A40A, and A67 to A86, or a pharmaceutically acceptable salt thereof, is wherein R^{ee} and R^{ff} are independently trifluoromethyl or 2,2,2-trifluoroethyl.
A96a. In embodiment A96a, the compound for use of any one of embodiments A2 to A39b, or a pharmaceutically acceptable salt thereof, is wherein the Degron is an E3 ubiquitin ligase ligand of formula (iii), (iv), (v), or (vi).
A96b. In embodiment A96b, the compound for use of any one of embodiments A2 to A39b, and A96a, or a pharmaceutically acceptable salt thereof, is wherein the Degron is an E3 ubiquitin ligase ligand of formula (iv) or (v).
A96c. In embodiment A96c, the compound for use of any one of embodiments A2 to A39b, A96a, and A96b, or a pharmaceutically acceptable salt thereof, is wherein R^{y}, R^{y1}, and R^{y2} are 1-fluorocycloprop-1-yl and W^{a} is bond, S, or methylene.
A96d. In embodiment A96d, the compound for use of any one of embodiments A3-1 to A39b and A96a to A96c, or a pharmaceutically acceptable salt thereof, is wherein W^{a} is S.
A97. In embodiment A97, the compound for use of any one of embodiments A3-1 to A96, or a pharmaceutically acceptable salt thereof, is wherein X¹, X², X³, and X⁴ are each a bond.
A98. In embodiment A98, the compound for use of any one of embodiments A3-1 to A96, or a pharmaceutically acceptable salt thereof, is wherein X¹, X², X³, and X⁴ are each independently selected from alkylene. In a subembodiment of embodiment A98, X¹, X², X³, and X⁴ are each methylene.
A99. In embodiment A99, the compound for use of any one of embodiments A3-1 to A96, or a pharmaceutically acceptable salt thereof, is wherein X¹, X², X³, and X⁴ are each —O—.
A100. In embodiment A100, the compound for use of any one of embodiments A3-1 to A96, or a pharmaceutically acceptable salt thereof, is wherein X¹, X², X³, and X⁴ are each independently selected from —(O-alkylene)-.
A101. In embodiment A101, the compound for use of any one of embodiments A3-1 to A96, or a pharmaceutically acceptable salt thereof, is wherein X¹, X², X³ and X⁴ are each independently selected from -(alkylene-O)—.
A102. In embodiment A102, the compound for use of any one of embodiments A3-1 to A96, or a pharmaceutically acceptable salt thereof, is wherein X¹, X², X³, and X⁴ are each independently selected from —(NR^{gg}-alkylene)-.
A103. In embodiment A103, the compound for use of any one of embodiments A3-1 to A96, or a pharmaceutically acceptable salt thereof, is wherein X¹, X², X³, and X⁴ are each independently selected from -(alkylene-NR^{hh})—.
A104. In embodiment A104, the compound for use of any one of embodiments A3-1 to A96, or a pharmaceutically acceptable salt thereof, is wherein X¹, X², X³, and X⁴ are each

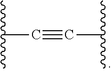 [SMILES: *C#C*]

A105. In embodiment A105, the compound for use of any one of embodiments A3-1 to A96, or a pharmaceutically acceptable salt thereof, is wherein X¹, X², X³, and X⁴ are each —NH—.
A106. In embodiment A106, the compound for use of any one of embodiments A3-1 to A96, or a pharmaceutically acceptable salt thereof, is wherein X¹, X², X³, and X⁴ are each independently selected from —N(alkyl)-. In a subembodiment of embodiment A106, X¹, X², X³, and X⁴ are each independently —N(methyl)- or —N(ethyl)-.
A107. In embodiment A107, the compound for use of any one of embodiments A3-1 to A96, or a pharmaceutically acceptable salt thereof, is wherein X¹, X², X³ and X⁴ are each —C(═O)—.
A108. In embodiment A108, the compound for use of any one of embodiments A3-1 to A96, or a pharmaceutically acceptable salt thereof, is wherein X¹, X², X³, and X⁴ are each independently —NR^{jj}C(═O)—.
A109. In embodiment A109, the compound for use of any one of embodiments A3-1 to A96, or a pharmaceutically acceptable salt thereof, is wherein X¹, X², X³, and X⁴ are each independently —C(═O)NR^{kk}—.
A110. In embodiment A110, the compound for use of any one of embodiments A3-1 to A96, A102, A103, A108, and A109, or a pharmaceutically acceptable salt thereof, is wherein R^{gg}, R^{hh}, R^{jj}, and R^{kk} are each independently hydrogen or alkyl.
A110a. In embodiment A110a, the compound for use of any one of embodiments A3-1 to A110 is wherein at least two of —Z¹—Z²—Z³—Z⁴—Z⁵—Z⁶— are not a bond. In a sub-embodiment of A110a, the compound for use is wherein at least three of —Z¹—Z²—Z³—Z⁴—Z⁵—Z⁶— are not a bond. In a sub-embodiment of A110a, the compound is wherein at least four of —Z¹—Z²—Z³—Z⁴—Z⁵—Z⁶— are not a bond.
A111. In embodiment A111, the compound for use of any one of embodiments A3-1 to A110a, or a pharmaceutically acceptable salt thereof, is wherein Z⁶ is —S(O)₂—.
A112. In embodiment A112, the compound for use of any one of embodiments A3-1 to A111, or a pharmaceutically acceptable salt thereof, is wherein Z⁵ is phenylene, monocyclic heteroarylene, or heterocycylene, where each ring is substituted with R^{q} and R^{r}.
A113. In embodiment A113, the compound for use of any one of embodiments A3-1 to A112, or a pharmaceutically acceptable salt thereof, is wherein Z⁵ is phenylene, monocyclic heteroarylene, or heterocycylene, where each ring is substituted with R^{q} and R^{r} and one and only one of Z¹ and X¹ is a bond, one and only one of Z¹ and X² is a bond, one and only one of Z¹ and X³, and one and only one of Z¹ and X⁴ is a bond (for sake of clarity, when X¹, X², X³, and X⁴ are not a bond, then X¹, X², X³, and X⁴ are as described in any one of embodiments A3-1 and A98 to A109).
A114. In embodiment A114, the compound for use of any one of embodiments A3-1 to A96d, or a pharmaceutically acceptable salt thereof, is wherein:
X¹, X², X³, and X⁴ are independently a bond, —(O-alkylene)-, —(NR^{gg}-alkylene)-,

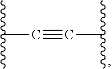 [SMILES: *C#C*]

or —N(alkyl)-, where R^{gg} is hydrogen or alkyl and each alkylene is independently optionally substituted with one or two fluoro (or X¹, X², X³, and X⁴ are absent in ligands (iii) to (vi));
Z¹ is a bond, alkylene, —(CO)NR—, —(O-alkylene)ₐ-, -(alkylene-O)ₐ—, phenylene, or heterocyclylene, where each ring is substituted with R^{h} and Rⁱ,
Z² is a bond, alkylene, —(O-alkylene)_{b}-, -(alkylene-O)_{b}—, cycloalkylene, or heterocyclylene, where each ring is substituted with R^{j} and R^{k};
Z³ is a bond, alkylene, —C(O)NR—, —NR′(CO)—, —O—, —NR″—, cycloalkylene, phenylene,
(alkylene)-phenylene-, -phenylene-(alkylene)-, monocyclic heteroarylene, -(alkylene)-monocyclic heteroarylene-, -monocyclic heteroarylene-(alkylene)-, heterocyclylene, -(alkylene)-heterocyclylene-, -heterocyclylene-(alkylene)-, bridged heterocyclylene, -(alkylene)-bridged heterocyclylene-, -bridged heterocyclylene-(alkylene)-, spiro heterocyclylene, -(alkylene)-spiro heterocyclylene-, -spiro heterocyclylene-(alkylene)-, or monocyclic heteroarylene, where each ring, by itself or as part of another group, is substituted with R^{m} and Rⁿ;
Z⁴ is a bond, -(alkylene-NR″)—, —(NR″-alkylene)-, —O—, —NR″—, cycloalkylene, phenylene, monocyclic heteroarylene, heterocyclylene, -(alkylene)-heterocyclylene-, -heterocyclylene-(alkylene)-, fused heterocyclylene, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p};
Z⁵ is a bond; and
Z⁶ is —S(O)₂—; and
wherein each alkylene in Z¹, Z², Z³, and Z⁴, by itself or as part of another group, is independently substituted with R^{s} and R^{t}.
A115. In embodiment A115, the compound for use of any one of embodiments A3-1 to A96d, or a pharmaceutically acceptable salt thereof, is wherein:
X¹, X², X³, X⁴, and Z¹ are each a bond;
Z² is a bond, alkylene, cycloalkylene, or heterocyclylene, where each ring is substituted with R^{j} and R^{k};
Z³ is a bond, alkylene, —C(O)NR—, —NR′(CO)—, —O—, —NR″—, cycloalkylene, phenylene, monocyclic heteroarylene, heterocyclylene, bicyclic heterocyclylene, bridged heterocyclylene, fused heterocyclylene, or spiro heterocyclylene, where each ring is substituted with R^{m} and Rⁿ;
Z⁴ is a bond, alkylene, —O—, cycloalkylene, phenylene, monocyclic heteroarylene, heterocyclylene, fused heterocyclylene, or spiro heterocyclylene, where each ring is substituted with R^{o} and R^{p};
Z⁵ is phenylene, monocyclic heteroarylene, or heterocycylene, where each ring is substituted with R^{q} and R^{r}; and
Z⁶ is —S(O)₂—; and
wherein each alkylene in Z², Z³, and Z⁴ is independently substituted with R^{s} and R^{t}.
A116. In embodiment A116, the compound for use of any one of embodiments A3-1 to A96d and A115, or a pharmaceutically acceptable salt thereof, is wherein:
X¹, X², X³, X⁴, Z¹, and Z² are each a bond;
Z³ is cycloalkylene, phenylene, monocyclic heteroarylene, heterocyclylene, bicyclic heterocyclylene, bridged heterocyclylene, fused heterocyclylene, or spiro heterocyclylene, where each ring is substituted with R^{m} and Rⁿ;
Z⁴ is a bond, alkylene, —O—, cycloalkylene, phenylene, monocyclic heteroarylene, heterocyclylene, fused heterocyclylene, or spiro heterocyclylene, where each ring is substituted with R^{o} and R^{p} independently selected from hydrogen, deuterium, alkyl, alkoxy, hydroxy, cyano, halo, haloalkyl, and haloalkoxy;
Z⁵ is phenylene, monocyclic heteroarylene, or heterocycylene, where each ring is substituted with R^{q} and R^{r}; and
Z⁶ is —S(O)₂—; and
wherein alkylene in Z⁴ is substituted with R^{s} and R^{t}.
A117. In embodiment A117, the compound for use of any one of embodiments A3-1 to A96d, A115, and A116, or a pharmaceutically acceptable salt thereof, is wherein:
X¹, X², X³, X⁴, Z¹, and Z² are each a bond;
Z³ is heterocyclylene, bicyclic heterocyclylene, bridged heterocyclylene, fused heterocyclylene, or spiro heterocyclylene, where each ring is substituted with R^{m} and Rⁿ independently selected from hydrogen, deuterium, alkyl, alkoxy, hydroxy, cyano, halo, haloalkyl, and haloalkoxy;
Z⁴ is alkylene, —O—, cycloalkylene, monocyclic heteroarylene, heterocyclylene, fused heterocyclylene, or spiro heterocyclylene, where each ring is substituted with R^{o} and R^{p} independently selected from hydrogen, deuterium, alkyl, alkoxy, hydroxy, cyano, halo, haloalkyl, and haloalkoxy;
Z⁵ is phenylene, monocyclic heteroarylene, or heterocycylene, where each ring is substituted with R^{q} and R^{r} independently selected from hydrogen, deuterium, alkyl, alkoxy, hydroxy, cyano, halo, haloalkyl, and haloalkoxy; and
Z⁶ is —S(O)₂—; and
wherein alkylene in Z⁴ is substituted with substituted with R^{s} and R^{t}.
A118. In embodiment A118, the compound for use of any one of embodiments A3-1 to A96d and A115 to A117, or a pharmaceutically acceptable salt thereof, is wherein:
X¹, X², X³, and X⁴, Z¹, and Z² are each a bond;
Z³ is heterocyclylene, bridged heterocyclylene, or spiro heterocyclylene, where each ring is substituted with R^{m} and Rⁿ independently selected from hydrogen, deuterium, alkyl, alkoxy, hydroxy, cyano, halo, haloalkyl, and haloalkoxy;
Z⁴ is alkylene, —O—, cycloalkylene, or heterocyclylene, where each ring is substituted with R^{o} and R^{p} independently selected from hydrogen, deuterium, alkyl, alkoxy, halo, haloalkyl, haloalkoxy, cyano, and hydroxy;
Z⁵ is phenylene, monocyclic heteroarylene, or heterocycylene, where each ring is substituted with R^{q} and R^{r} independently selected from hydrogen, deuterium, alkyl, alkoxy, hydroxy, cyano, halo, haloalkyl, and haloalkoxy; and
Z⁶ is —S(O)₂—; and
wherein alkylene in Z⁴ is substituted with R^{s} and R^{t}.
A119. In embodiment A119, the compound for use of any one of embodiments A3-1 to A96d and A115 to A118, or a pharmaceutically acceptable salt thereof, is wherein:
X¹, X², X³, X⁴, Z¹, and Z² are each a bond;
Z³ is heterocyclylene, bridged heterocyclylene, or spiro heterocyclylene, where each ring is substituted with R^{m} and R^{b} independently selected from hydrogen, deuterium, alkyl, alkoxy, hydroxy, cyano, halo, haloalkyl, and haloalkoxy;
Z⁴ is alkylene, —O—, cycloalkylene, or heterocyclylene, where each ring is substituted with R^{o} and R^{p} independently selected from hydrogen, deuterium, alkyl, alkoxy, halo, haloalkyl, haloalkoxy, cyano, and hydroxy, preferably hydrogen, deuterium, alkyl, alkoxy, halo, haloalkyl, and haloalkoxy, (in a subembodiment Z⁴ is alkylene or —O—);
Z⁵ is phenylene or monocyclic heteroarylene, each ring substituted with R^{q} and R^{r} independently selected from hydrogen, deuterium, alkyl, alkoxy, hydroxy, cyano, halo, haloalkyl, and haloalkoxy; and
Z⁶ is —S(O)₂—; and
wherein alkylene in Z⁴ is substituted with R^{s} and R^{t}.
A120. In embodiment A120, the compound for use of any one of embodiments A3-1 to A96d and A115, or a pharmaceutically acceptable salt thereof, is wherein:
X¹, X², X³, X⁴, and Z¹ are each a bond;
Z² is cycloalkylene or heterocyclylene, where each ring is substituted with R^{j} and R^{k} independently selected from hydrogen, deuterium, alkyl, alkoxy, halo, haloalkyl, and haloalkoxy;
Z³ is cycloalkylene, phenylene, monocyclic heteroarylene, heterocyclylene, bicyclic heterocyclylene, bridged heterocyclylene, fused heterocyclylene, or spiro heterocyclylene, where each ring is substituted with R^{m} and Rⁿ independently selected from hydrogen, deuterium, alkyl, alkoxy, hydroxy, cyano, halo, haloalkyl, and haloalkoxy;
Z⁴ is a bond, alkylene, or —O—;
Z⁵ is phenylene, monocyclic heteroarylene (e.g., pyridindiyl), or heterocycylene, where each ring is substituted with R^{m} and Rⁿ independently selected from hydrogen, deuterium, alkyl, alkoxy, hydroxy, cyano, halo, haloalkyl, and haloalkoxy; and
Z⁶ is —S(O)₂—; and
wherein alkylene in Z⁴ is substituted with R^{s} and R^{t}.
A121. In embodiment A121, the compound for use of any one of embodiments A3-1 to A96d, A111, and A115, or a pharmaceutically acceptable salt thereof, is wherein:
X¹, X², X³, X⁴, and Z¹ are each a bond;
Z² is heterocyclylene substituted with R^{j} and R^{k} independently selected from hydrogen, deuterium, alkyl, alkoxy, halo, haloalkyl, and haloalkoxy;
Z³ is heterocyclylene substituted with R^{m} and Rⁿ independently selected from hydrogen, deuterium, alkyl, alkoxy, hydroxy, cyano, halo, haloalkyl, and haloalkoxy;
Z⁴ is a bond, alkylene, or —O—;
Z⁵ is phenylene or monocyclic heteroarylene, each ring substituted with R^{q} and R^{r} independently selected from hydrogen, deuterium, alkyl, alkoxy, hydroxy, cyano, halo, haloalkyl, and haloalkoxy; and
Z⁶ is —S(O)₂—; and
wherein alkylene in Z⁴ is substituted with R^{s} and R^{t}.
A122. In embodiment A122, the compound for use of any one of embodiments A3-1 to A96d, A97, A111, and A112, or a pharmaceutically acceptable salt thereof, is wherein:
X¹, X², X³, X⁴, and Z¹ are each a bond;
Z² is heterocyclylene substituted with R^{j} and R^{k}, preferably R^{j} and R^{k} are independently selected from hydrogen, deuterium, alkyl, alkoxy, halo, haloalkyl, and haloalkoxy;
Z³ is a bond, alkylene, or —O—;
Z⁴ is heterocyclylene, bridged heterocyclylene, or spiro heterocyclylene, where each ring is substituted with R^{o} and R^{p}, preferably R^{o} and R^{p} are independently selected from hydrogen, deuterium, alkyl, alkoxy, hydroxy, cyano, halo, haloalkyl, and haloalkoxy;
Z⁵ is phenylene or monocyclic heteroarylene, each ring substituted with R^{q} and R^{r}, preferably R^{q} and R^{r} are independently selected from hydrogen, deuterium, alkyl, alkoxy, hydroxy, cyano, halo, haloalkyl, and haloalkoxy; and
Z⁶ is —S(O)₂—; and
wherein alkylene in Z³ is substituted with R^{s} and R^{t}.
A123. In embodiment A123, the compound for use of any one of embodiments A3-1 to A96d, or a pharmaceutically acceptable salt thereof, is wherein Z⁴ is heterocyclylene or spiro heterocyclylene, where each ring is substituted with R^{o} and R^{p}, preferably R^{o} and R^{p} are independently selected from hydrogen, deuterium, alkyl, alkoxy, hydroxy, cyano, halo, haloalkyl, and haloalkoxy.
A124. In embodiment A124, the compound for use of any one of embodiments A3-1 to A96d, A115, and A116 or a pharmaceutically acceptable salt thereof, is wherein:
X¹, X², X³, X⁴, Z¹ and Z² are each a bond;
Z³ is heterocyclylene, where each ring is substituted with R^{m} and Rⁿ independently selected from hydrogen, deuterium, alkyl, alkoxy, hydroxy, cyano, halo, haloalkyl, and haloalkoxy;
Z⁴ is cycloalkylene substituted with R^{o} and R^{p} independently selected from hydrogen, alkyl, alkoxy, halo, haloalkyl, and haloalkoxy;
Z⁵ is phenylene or monocyclic heteroarylene, each ring substituted with R^{q} and R^{r} independently selected from hydrogen, deuterium, alkyl, alkoxy, hydroxy, cyano, halo, haloalkyl, and haloalkoxy; and
Z⁶ is —S(O)₂—.
A125. In embodiment A125, the compound for use of any one of embodiments A3-1 to A96d, or a pharmaceutically acceptable salt thereof, is wherein one and only one of X¹ and Z¹, or one and only one of X² and Z¹, or one and only one of X³ and Z¹, or one and only one of X⁴ and Z¹ is a bond.
A126. In embodiment A126, the compound for use of any one of embodiments A3-1 to A96d, or a pharmaceutically acceptable salt thereof, is wherein X¹, X², X³, X⁴, and Z¹ are each a bond.
A127. In embodiment A127, the compound for use of any one of embodiments A3-1 to A96d, A125, and A126, or a pharmaceutically acceptable salt thereof, is wherein Z² is heterocyclylene or bridged heterocyclylene, each ring substituted with R^{j} and R^{k}
A128. In embodiment A128, the compound for use of any one of embodiments A3-1 to A96d, A125, and A126, or a pharmaceutically acceptable salt thereof, is wherein Z² is a bond.
A129. In embodiment A129, the compound for use of any one of embodiments A3-1 to A96d, and A125 to A128, or a pharmaceutically acceptable salt thereof, is wherein:
Z³ is alkylene, cycloalkylene, phenylene, -(alkylene)-phenylene-, -phenylene-(alkylene)-, monocyclic heteroarylene, -(alkylene)-monocyclic heteroarylene-, -monocyclic heteroarylene-(alkylene)-, heterocyclylene, -(alkylene)-heterocyclylene-, -heterocyclylene-(alkylene)-, bicyclic heterocyclylene, bridged heterocyclylene, -(alkylene)-bridged heterocyclylene-, -bridged heterocyclylene-(alkylene)-, fused heterocyclylene, spiro heterocyclylene, -(alkylene)-spiro heterocyclylene-, or -spiro heterocyclylene-(alkylene), where each ring, by itself or as part of another group, is substituted with R^{m} and Rⁿ;
Z⁴ is alkylene, -(alkylene-NR″)—, —(NR″-alkylene)-, —O—, —NR″—, —(O-alkylene)_{d}-, -(alkylene-O)_{d}—, cycloalkylene, -(alkylene)-cycloalkylene-, -cycloalkylene-(alkylene)-, spiro cyclolalkylene, phenylene, heteroarylene, heterocyclylene, -(alkylene)-heterocyclylene-, -heterocyclylene-(alkylene)-, fused heterocyclylene, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p};
Z⁵ is a bond, -alkylene, —NR″—, —O—, —C(O)—, —S(O)₂—, —NR′(CO)—, —C(O)NR—, phenylene, monocyclic heteroarylene, or heterocycylene, where each ring is substituted with R^{q} and R^{r}; and
Z⁶ is a bond, alkylene, —NR″—, —O—, -(alkylene-O)—, —C(O)—, —S(O)₂—, —NR′(CO)—, or —C(O)NR—; and
and each alkylene in Z³, Z⁴, Z⁵, and Z⁶, itself or as part of another group, is independently substituted with R^{s} and R^{t}.
A130. In embodiment A130, the compound for use of any one of embodiments A3-1 to A96d, and A125 to A129, or a pharmaceutically acceptable salt thereof, is wherein:
Z³ is alkylene, cycloalkylene, phenylene, -(alkylene)-phenylene-, -phenylene-(alkylene)-, monocyclic heteroarylene, -(alkylene)-monocyclic heteroarylene-, -monocyclic heteroarylene-(alkylene)-, heterocyclylene, -(alkylene)-heterocyclylene-, -heterocyclylene-(alkylene)-, bicyclic heterocyclylene, bridged heterocyclylene, -(alkylene)-bridged heterocyclylene-, -bridged heterocyclylene-(alkylene)-, fused heterocyclylene, spiro heterocyclylene, -(alkylene)-spiro heterocyclylene-, or -spiro heterocyclylene-(alkylene), where each ring, by itself or as part of another group, is substituted with R^{m} and Rⁿ;
Z⁴ is alkylene, -(alkylene-NR″)—, —(NR″-alkylene)-, —O—, —NR″—, —(O-alkylene)_{d}-, -(alkylene-O)—, cycloalkylene, -(alkylene)-cycloalkylene-, -cycloalkylene-(alkylene)-, spiro cyclolalkylene, phenylene, heteroarylene, heterocyclylene, -(alkylene)-heterocyclylene-, -heterocyclylene-(alkylene)-, fused heterocyclylene, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p};
Z⁵ is a bond, -alkylene, —NR″—, —O—, —C(O)—, —S(O)₂—, —NR′(CO)—, —C(O)NR—, phenylene, monocyclic heteroarylene, or heterocycylene, where each ring is substituted with R^{q} and R^{r}; and
Z⁶ is —S(O)₂—; and
and each alkylene in Z³, Z⁴, and Z⁵, itself or as part of another group, is independently substituted with R^{s} and R^{t}.
A130A. In embodiment A130A, the compound for use of any one of embodiments A3-1, A3A to A97, A111-A113, A126, and A128-A130, or a pharmaceutically acceptable salt thereof, is wherein:
X¹, X², X³, X⁴, Z¹, and Z² are each a bond;
Z³ is-heterocyclylene-(alkylene)-, where heterocyclylene is substituted with R^{m} and Rⁿ and alkylene is substituted with R^{s} and R^{t};
Z⁴ is phenylene or monocyclic heteroarylene, where each ring is substituted with R^{c} and R^{p};
Z⁵ is phenylene substituted with R^{q} and R^{r}; and
Z⁶ is —S(O)₂—.
A131. In embodiment A131, the compound for use of any one of embodiments A3-1 to A96d, and A125 to A130, or a pharmaceutically acceptable salt thereof, is wherein:
Z³ is alkylene, phenylene, -(alkylene)-phenylene-, -phenylene-(alkylene)-, monocyclic heteroarylene, -(alkylene)-monocyclic heteroarylene-, -monocyclic heteroarylene-(alkylene)-, heterocyclylene, -(alkylene)-heterocyclylene-, -heterocyclylene-(alkylene)-, bridged heterocyclylene, -(alkylene)-bridged heterocyclylene-, -bridged heterocyclylene-(alkylene)-, spiro heterocyclylene, -(alkylene)-spiro heterocyclylene-, or -spiro heterocyclylene-(alkylene), where each ring, by itself or as part of another group, is substituted with R^{m} and Rⁿ.
Z⁴ is alkylene, -(alkylene-NR″)—, —(NR″-alkylene)-, —O—, —NR″—, —(O-alkylene)_{d}-, -(alkylene-O)_{d}—, cycloalkylene, -(alkylene)-cycloalkylene-, -cycloalkylene-(alkylene)-, spiro cyclolalkylene, phenylene, heteroarylene, heterocyclylene, -(alkylene)-heterocyclylene-, -heterocyclylene-(alkylene)-, fused heterocyclylene, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p};
Z⁵ is phenylene, monocyclic heteroarylene, or heterocycylene, where each ring is substituted with R^{q} and R^{r}; and
Z⁶ is —S(O)₂—; and
and each alkylene in Z³ and Z⁴, itself or as part of another group, is independently substituted with R^{s} and R^{t}.
A131a. In embodiment A131a, the compound for use of any one of embodiments A3-1 to A96d, and A125 to A130, or a pharmaceutically acceptable salt thereof, is wherein:
Z³ is alkylene, phenylene, -(alkylene)-phenylene-, -phenylene-(alkylene)-, monocyclic heteroarylene, -(alkylene)-monocyclic heteroarylene-, -monocyclic heteroarylene-(alkylene)-, where each ring, by itself or as part of another group, is substituted with R^{m} and Rⁿ;
Z⁴ is alkylene, -(alkylene-NR″)—, —(NR″-alkylene)-, —O—, —NR″—, —(O-alkylene)_{d}-, -(alkylene-O)—, cycloalkylene, -(alkylene)-cycloalkylene-, -cycloalkylene-(alkylene)-, spiro cyclolalkylene, phenylene, heteroarylene, heterocyclylene, -(alkylene)-heterocyclylene-, -heterocyclylene-(alkylene)-, fused heterocyclylene, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p};
Z⁵ is phenylene, monocyclic heteroarylene, or heterocycylene, where each ring is substituted with R^{q} and R^{r}; and
Z⁶ is —S(O)₂; and
and each alkylene in Z³ and Z⁴, itself or as part of another group, is independently substituted with R^{s} and R^{t}.
A132. In embodiment A132, the compound for use of any one of embodiments A3-1 to A96d, and A125 to A131, or a pharmaceutically acceptable salt thereof, is wherein:
Z³ is heterocyclylene, -(alkylene)-heterocyclylene-, -heterocyclylene-(alkylene)-, bridged heterocyclylene, -(alkylene)-bridged heterocyclylene-, -bridged heterocyclylene-(alkylene)-, spiro heterocyclylene, -(alkylene)-spiro heterocyclylene-, or -spiro heterocyclylene-(alkylene), where each ring, by itself or as part of another group, is substituted with R^{m} and Rⁿ;
Z⁴ is alkylene, -(alkylene-NR″)—, —(NR″-alkylene)-, —O—, —NR″—, —(O-alkylene)_{d}-, -(alkylene-O)_{d}—, phenylene, heteroarylene, heterocyclylene, -(alkylene)-heterocyclylene-, -heterocyclylene-(alkylene)-, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p};
Z⁵ is phenylene, monocyclic heteroarylene, or heterocycylene, where each ring is substituted with R^{q} and R^{r}; and
Z⁶ is —S(O)₂; and
and each alkylene in Z³ and Z⁴, itself or as part of another group, is independently substituted with R^{s} and R^{t}.
A132A. In embodiment A132A, the compound for use of A132, or a pharmaceutically acceptable salt thereof, is wherein:
X¹, X², X³, X⁴, Z¹, and Z² are each a bond;
Z³ is-heterocyclylene-(alkylene)-, where heterocyclylene is substituted with R^{m} and Rⁿ and where alkylene is independently substituted with R^{s} and R^{t};
Z⁴ is phenylene or monocyclic heteroarylene, where each ring is substituted with R^{o} and R^{p};
Z⁵ is phenylene substituted with R^{q} and R^{r}; and
Z⁶ is —S(O)₂.
A133. In embodiment A133, the compound for use of any one of embodiments A3-1 to A96d, A125 to A131, and A132, or a pharmaceutically acceptable salt thereof, is wherein:
Z³ is heterocyclylene, -(alkylene)-heterocyclylene-, -heterocyclylene-(alkylene)-, bridged heterocyclylene, -(alkylene)-bridged heterocyclylene-, -bridged heterocyclylene-(alkylene)-, spiro heterocyclylene, -(alkylene)-spiro heterocyclylene-, or -spiro heterocyclylene-(alkylene), where each ring, by itself or as part of another group, is substituted with R^{m} and Rⁿ;
Z⁴ is alkylene, —O—, heterocyclylene, -(alkylene)-heterocyclylene-, -heterocyclylene-(alkylene)-, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p} independently selected from hydrogen, deuterium, alkyl, alkoxy, halo, haloalkyl, haloalkoxy, cyano, hydroxy, amino, alkylamino, and dialkylamino;
Z⁵ is phenylene, monocyclic heteroarylene, or heterocycylene, where each ring is substituted with R^{q} and R^{r} independently selected from hydrogen, deuterium, alkyl, alkoxy, halo, haloalkyl, haloalkoxy, cyano, hydroxy, amino, alkylamino, and dialkylamino; and
Z⁶ is —S(O)₂; and
and each alkylene in Z³ and Z⁴, itself or as part of another group, is independently substituted with R^{s} and R^{t}.
A134. In embodiment A134, the compound for use of any one of embodiments A3-1 to A96d, A125 to A131, and A132 to A133, or a pharmaceutically acceptable salt thereof, is wherein:
Z³ is heterocyclylene, bridged heterocyclylene, or spiro heterocyclylene, where each ring is substituted with R^{m} and Rⁿ;
Z⁴ is alkylene, —O—, heterocyclylene, -(alkylene)-heterocyclylene-, -(alkylene)-bridged heterocyclylene-, where each ring, by itself or as part of another group, is substituted with R^{o} and R^{p};
Z⁵ is phenylene, monocyclic heteroarylene, or heterocycylene, where each ring is substituted with R^{q} and R^{r}; and
Z⁶ is —S(O)₂; and
and each alkylene in Z⁴, itself or as part of another group, is substituted with R^{s} and R^{t}.
A135. In embodiment A135, the compound for use of any one of embodiments A3-1 to A113 and A115 to A134, or a pharmaceutically acceptable salt thereof, is wherein —Z⁵— is

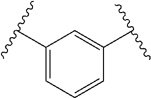 [SMILES: *c1cccc(*)c1]

(i.e., Z⁵ is phenylene where Z⁴ and Z⁶ are attached at meta position of the phenylene ring) substituted with R^{q} and R^{r} independently selected from hydrogen, deuterium, alkyl, alkoxy, halo, haloalkyl, and haloalkoxy.
A136. In embodiment A136, the compound for use of any one of embodiments A3-1 to A113 and A115 to A135, or a pharmaceutically acceptable salt thereof, is wherein —Z⁵— is

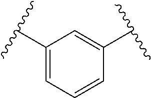 [SMILES: *c1cccc(*)c1]

substituted with R^{q} and R^{r} independently selected from hydrogen, deuterium, methyl, methoxy, fluoro, chloro, difluoromethyl, trifluoromethyl, difluoromethoxy, and trifluoromethoxy.
A137. In embodiment A137, the compound for use of any one of embodiments A3-1 to A113 and A115 to A136, or a pharmaceutically acceptable salt thereof, is wherein —Z⁵— is

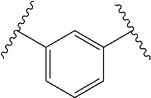 [SMILES: *c1cccc(*)c1]

substituted with R^{q} and R^{r} independently selected from hydrogen, deuterium, or fluoro.
A137a. In embodiment A137a, the compound for use of any one of embodiments A3-1 to A113 and A115 to A137, or a pharmaceutically acceptable salt thereof, is wherein —Z⁵— is

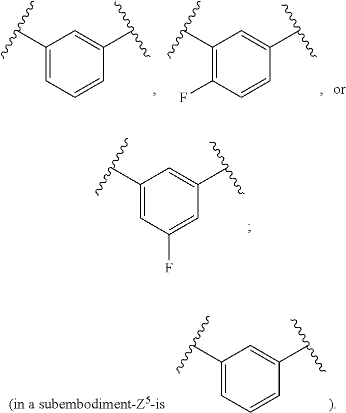 [SMILES: *c1cccc(*)c1 ; *c1ccc(F)c(*)c1 ; *c1cc(*)cc(F)c1 ; *c1cccc(*)c1]

A137b. In embodiment A137b, the compound for use of any one of embodiments A3-1 to A113 and A115 to A136, or a pharmaceutically acceptable salt thereof, is wherein —Z⁵— is

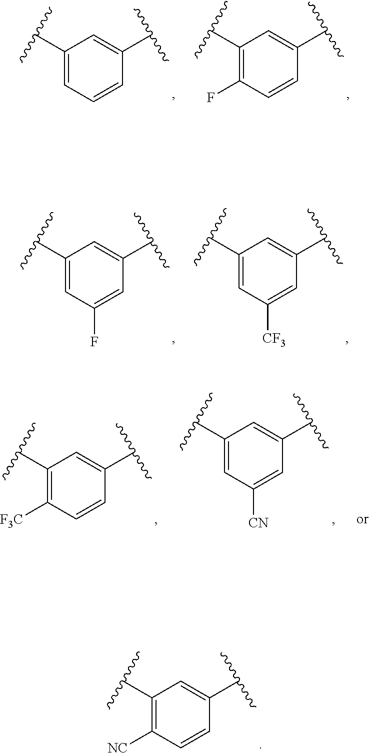

A138. In embodiment A138, the compound for use of any one of embodiments A3-1 to A113, A115 to A132, A133, and A134, or a pharmaceutically acceptable salt thereof, is wherein-Z⁵— is monocyclic heteroarylene (such as imidazol-1,5-diyl, pyridin-2,4-diyl, pyridin-2,6-diyl, or pyridin-3,5-diyl) substituted with R^{q} and R^{r} independently selected from hydrogen, alkyl, alkoxy, halo, haloalkyl, and haloalkoxy.
A139. In embodiment A139, the compound for use of any one of embodiments A3-1 to A113, A115 to A132, A133, A134, and A138, or a pharmaceutically acceptable salt thereof, is wherein —Z⁵— is imidazol-2,5-diyl, pyridin-2,4-diyl, pyridin-2,6-diyl, or pyridin-3,5-diyl, each ring substituted with R^{q} and R^{r} independently selected from hydrogen, methyl, methoxy, fluoro, chloro, difluoromethyl, trifluoromethyl, 2,2,2-trifluoroethyl, difluoromethoxy, and trifluoromethoxy.
A140. In embodiment A140, the compound for use of any one of embodiments A3-1 to A113, A115 to A132, A133, A134, A138, and A139, or a pharmaceutically acceptable salt thereof, is wherein —Z⁵— is imidazol-2,5-diyl, pyridin-2,4-diyl, pyridin-2,6-diyl, or pyridin-3,5-diyl, each ring substituted with R^{q} and R^{r} independently selected from hydrogen, methyl, methoxy, fluoro, chloro, difluoromethyl, trifluoromethyl, difluoromethoxy, and trifluoromethoxy.
A141. In embodiment A141, the compound for use of any one of embodiments A3-1 to A113, A115 to A118, A120, A123, and A125 to A132, A133, and A134, or a pharmaceutically acceptable salt thereof, is wherein —Z⁵— is heterocyclylene substituted with R^{q} and R^{r} independently selected from hydrogen, methyl, methoxy, fluoro, chloro, difluoromethyl, trifluoromethyl, 2,2,2-trifluoroethyl, difluoromethoxy, and trifluoromethoxy.
A142. In embodiment A142, the compound for use of any one of embodiments A3-1 to A113, A115 to A118, A120, A123, A125 to A132, A133, A134, and A141, or a pharmaceutically acceptable salt thereof, is wherein —Z⁵— is azetidinyl, pyrrolidinyl, piperazinyl, or piperidinyl.
A143. In embodiment A143, the compound for use of any one of embodiments A3-1 to A134, or a pharmaceutically acceptable salt thereof, is wherein each alkylene of Z¹, Z², Z³, Z⁴, Z⁵, and Z⁶, by itself and when present, is methylene, ethylene, or propylene, each substituted with R^{s} and R^{t}.
A144. In embodiment A144, the compound for use of any one of embodiments A3-1 to A134 and A143, or a pharmaceutically acceptable salt thereof, is wherein each alkylene of Z¹, Z², Z³, Z⁴, Z⁵, and Z⁶, by itself and when present, is methylene substituted with R^{s} and R^{t}.
A145. In embodiment A145, the compound for use of any one of embodiments A3-1 to A134, or a pharmaceutically acceptable salt thereof, is wherein each alkylene of Z¹, Z², Z³, Z⁴, Z⁵, and Z⁶, by itself or as part of —(O-alkylene)ₐ- in Z¹, -(alkylene-O)ₐ— in Z¹, —(O-alkylene)_{b}- in Z², -(alkylene-O)_{b}— in Z², —(O-alkylene)_{c}- in Z³, -(alkylene-O)_{c}— in Z³, —(O-alkylene)_{b}- in Z⁴, and -(alkylene-O)_{d}— in Z⁴, and -(alkylene-O)— in Z⁶ and when present, is ethylene or propylene; as part of -(alkylene-NR″)— and —(NR″-alkylene)- and when present, is methylene, ethylene or propylene; and as part of -(alkylene)-cycloalkylene-, -cycloalkylene-(alkylene)-, -(alkylene)-phenylene-, -phenylene-(alkylene)-, -(alkylene)-monocyclic heteroarylene-, -monocyclic heteroarylene-(alkylene)-, -(alkylene)-heterocyclylene-, -heterocyclylene-(alkylene)-, -(alkylene)-bridged heterocyclylene-, -bridged heterocyclylene-(alkylene)-, -(alkylene)-spiro heterocyclylene-, and -spiro heterocyclylene-(alkylene)- is methylene, ethylene, propylene, or butylene, wherein each of above alkylene group is substituted with R^{s} and R^{t}.
A146. In embodiment A146, the compound for use of any one of embodiments A3-1 to A134 and A145, or a pharmaceutically acceptable salt thereof, is wherein each alkylene of Z¹, Z², Z³, Z⁴, Z⁵, and Z⁶, by itself or as part of —(O-alkylene)ₐ- in Z¹, -(alkylene-O)ₐ— in Z¹, —(O-alkylene)_{b}- in Z², -(alkylene-O)_{b}— in Z², —(O-alkylene)_{c}- in Z³, -(alkylene-O)_{c}— in Z³, —(O-alkylene)ₐ- in Z⁴, and -(alkylene-O)_{d}— in Z⁴, and -(alkylene-O)— in Z⁶, and when present, is ethylene; as part of -(alkylene-NR″)—) and —(NR″-alkylene)- and when present, is methylene; and as part of -(alkylene)-cycloalkylene-, -cycloalkylene-(alkylene)-, -(alkylene)-phenylene-, -phenylene-(alkylene)-, -(alkylene)-monocyclic heteroarylene-, -monocyclic heteroarylene-(alkylene)-, -(alkylene)-heterocyclylene-, -heterocyclylene-(alkylene)-, -(alkylene)-bridged heterocyclylene-, -bridged heterocyclylene-(alkylene)-, -(alkylene)-spiro heterocyclylene-, and -spiro heterocyclylene-(alkylene)- and when present, is methylene.
A147. In embodiment A147, the compound for use of any one of embodiments A3-1 to A146, or a pharmaceutically acceptable salt thereof, is wherein each R, R′ and R″ of Z¹, Z², Z³, Z⁴, Z⁵, and Z⁶, when present, is independently hydrogen or methyl.
A148. In embodiment A148, the compound for use of any one of embodiments A3-1 to A147, or a pharmaceutically acceptable salt thereof, is wherein each R, R′ and R″ of Z¹, Z², Z³, Z⁴, Z⁵, and Z⁶, when present, is hydrogen.
A149. In embodiment A149, the compound for use of any one of embodiments A1 to A147, or a pharmaceutically acceptable salt thereof, is wherein each R, R′ and R″ of Z¹, Z², Z³, Z⁴, Z⁵, and Z⁶, when present, is methyl.
A150. In embodiment A150, the compound for use of any one of embodiments A3-1 to A149, or a pharmaceutically acceptable salt thereof, is wherein each cycloalkylene of Z², Z³, and Z⁴, when present, is independently selected from cyclopropylene, cyclobutylene, cyclopentylene, and cyclohexylene.
A151. In embodiment A151, the compound for use of any one of embodiments A3-1 to A150, or a pharmaceutically acceptable salt thereof, is wherein each cycloalkylene of Z², Z³, and Z⁴, when present, is independently selected from 1,3-cyclopentylene, 1,3-cyclohexylene, and 1,4-cyclohexylene.
A152. In embodiment A152, the compound for use of any one of embodiments A3-1 to A151, or a pharmaceutically acceptable salt thereof, is wherein heteroarylene is monocyclic heteroarylene and each monocyclic heteroarylene of Z¹, Z³, Z⁴, and Z⁵, when present, is independently selected from imidazoldiyl, pyridindiyl and pyrimidindiyl unless stated otherwise in any of the embodiments above.
A153. In embodiment A153, the compound for use of any one of embodiments A3-1 to A152, or a pharmaceutically acceptable salt thereof, is wherein heteroarylene is monocyclic heteroarylene and each monocyclic heteroarylene of Z¹, Z³, Z⁴, and Z⁵, when present, is independently selected from imidazol-2,5-diyl, pyridin-2,4-diyl, pyridin-2,6-diyl, and pyridin-3,5-diyl, unless stated otherwise in any of the embodiments above.
A154. In embodiment A154, the compound for use of any one of embodiments A3-1 to A153, or a pharmaceutically acceptable salt thereof, is wherein each phenylene of Z¹, Z³, Z⁴, and Z⁵, when present, is independently selected from 1,3-phenylene and 1,4-phenylene unless stated otherwise in any of the embodiments above.
A155. In embodiment A155, the compound for use of any one of embodiments A3-1 to A154, or a pharmaceutically acceptable salt thereof, is wherein each heterocyclylene, bridged heterocyclylene, and spiro heterocyclylene, of Z¹, Z², Z³, Z⁴, and Z⁵, when present, are independently selected from:

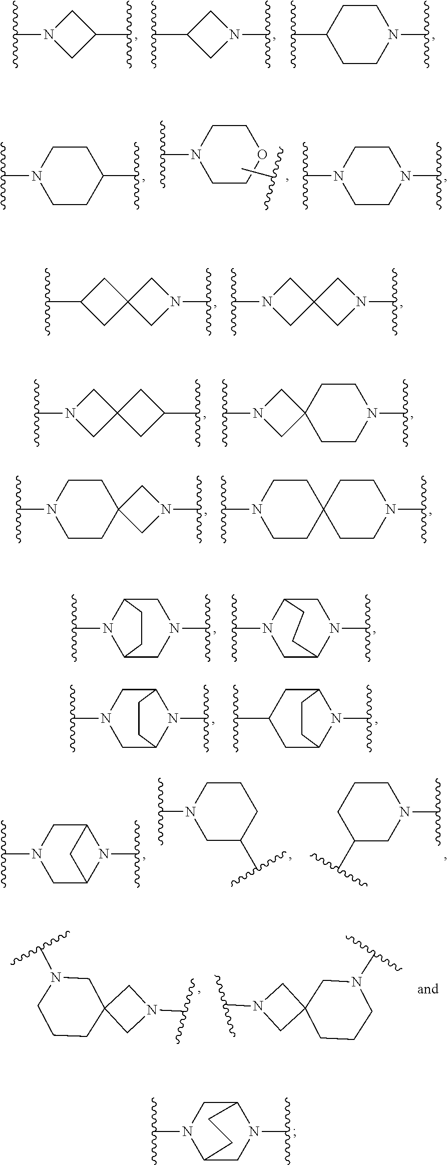

wherein each ring is optionally substituted with 1, 2, or 3 fluoro, unless stated otherwise in any of the embodiments above.
A156. In embodiment A156, the compound for use of any one of embodiments A3-1 to A155, or a pharmaceutically acceptable salt thereof, is wherein each heterocyclylene, bridged heterocyclylene, and spiro heterocyclylene, of Z¹, Z², Z³, Z⁴, and Z⁵, when present, are independently selected from:

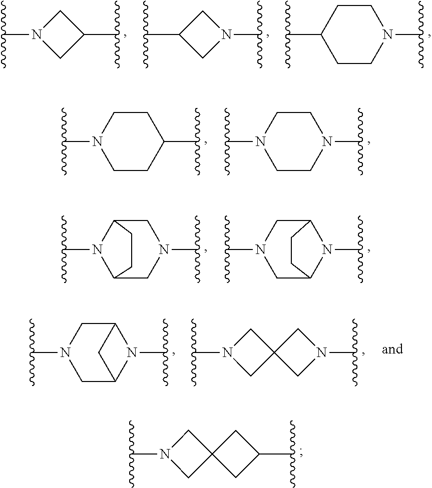

wherein each ring is optionally substituted with 1 or 2 fluoro, unless stated otherwise in any of the embodiments above.
A157. In embodiment A157, the compound for use of any one of embodiments A3-1 to A96d, or a pharmaceutically acceptable salt thereof, is wherein L (when the Degron is a group of formula (iii) to (vi)), —X¹-L-, —X²-L-, —X³-L- and —X⁴-L- (when the Degron is a group of formula (i) or (ii)) are independently:

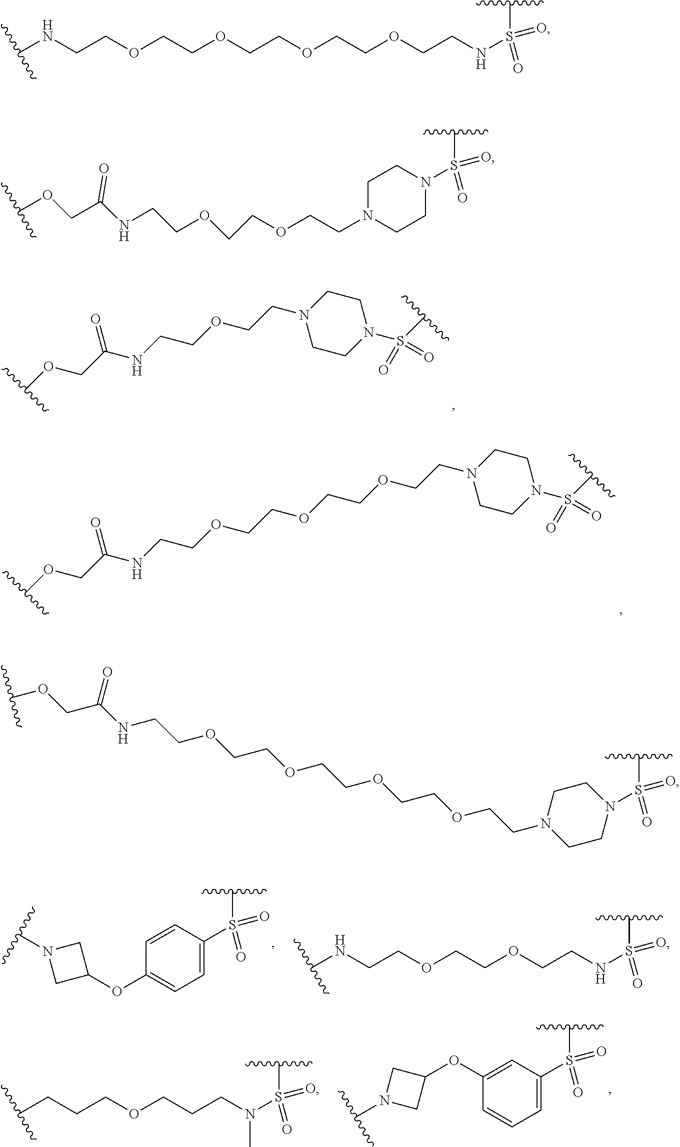

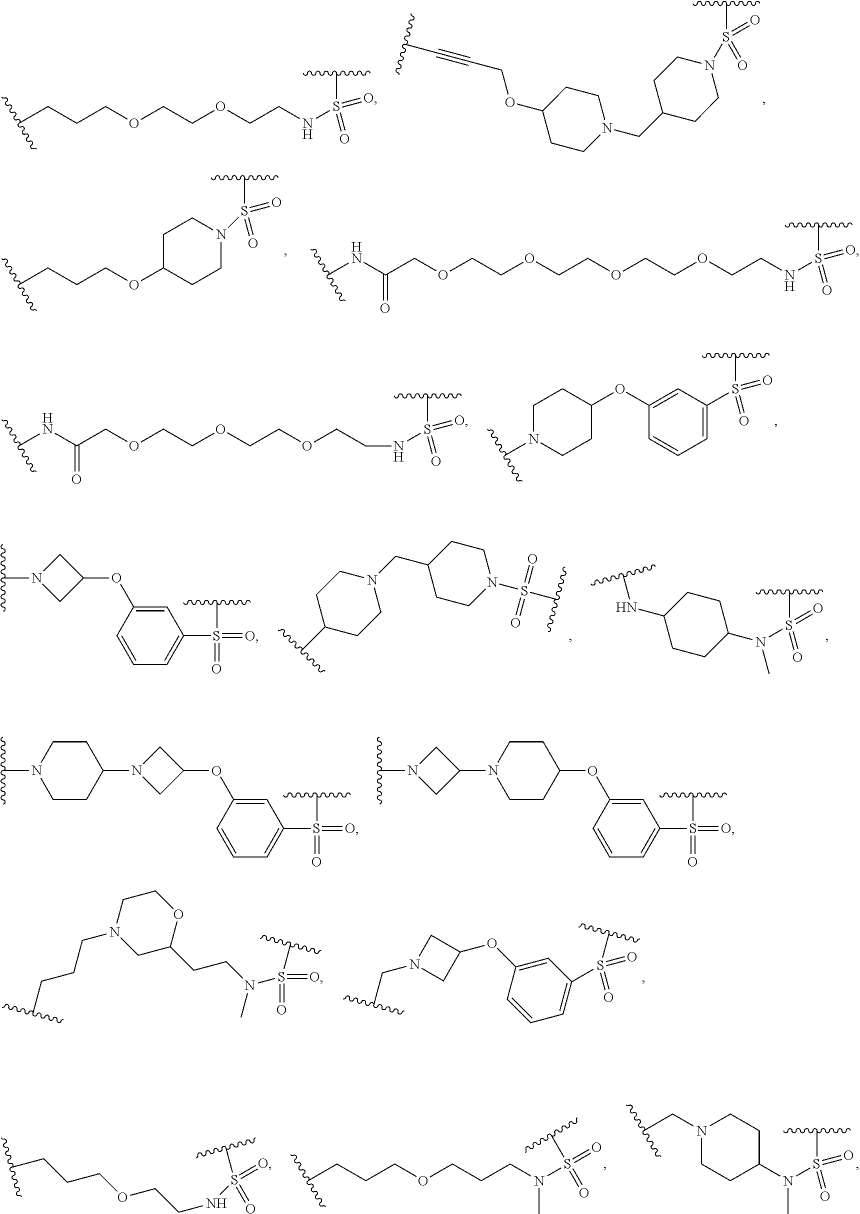

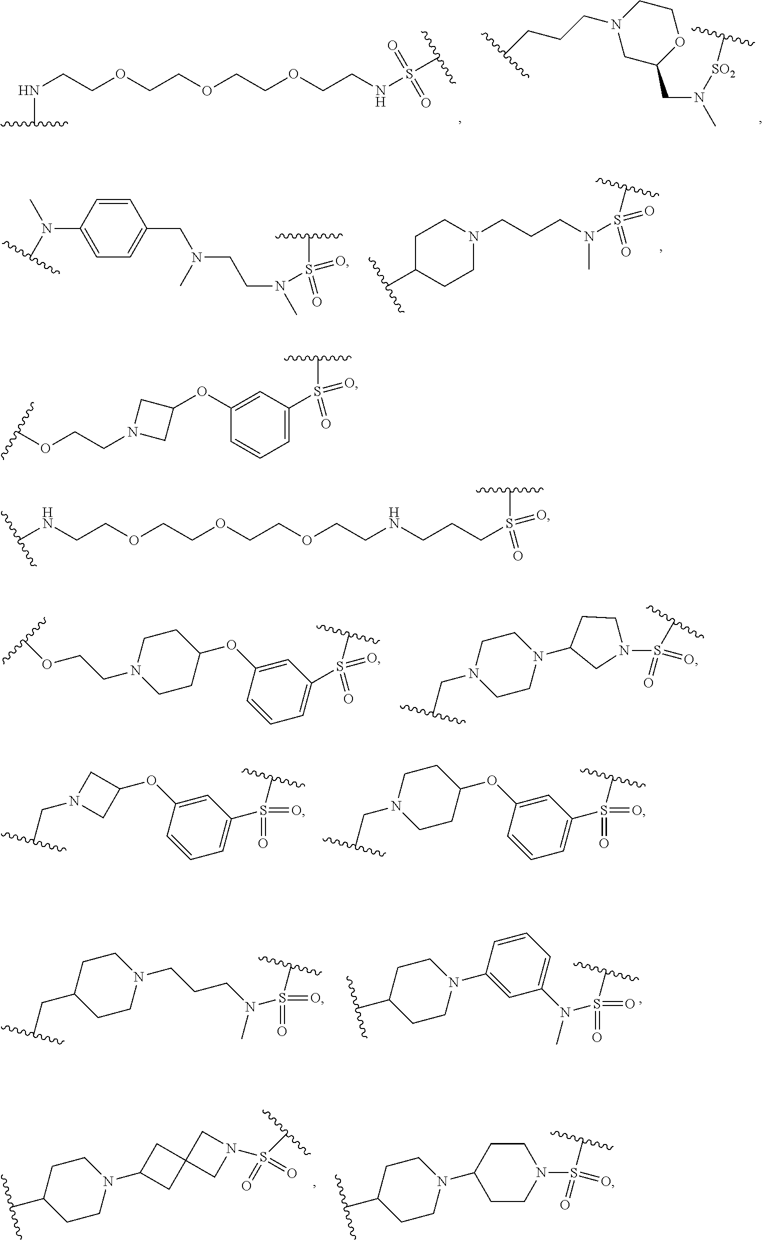

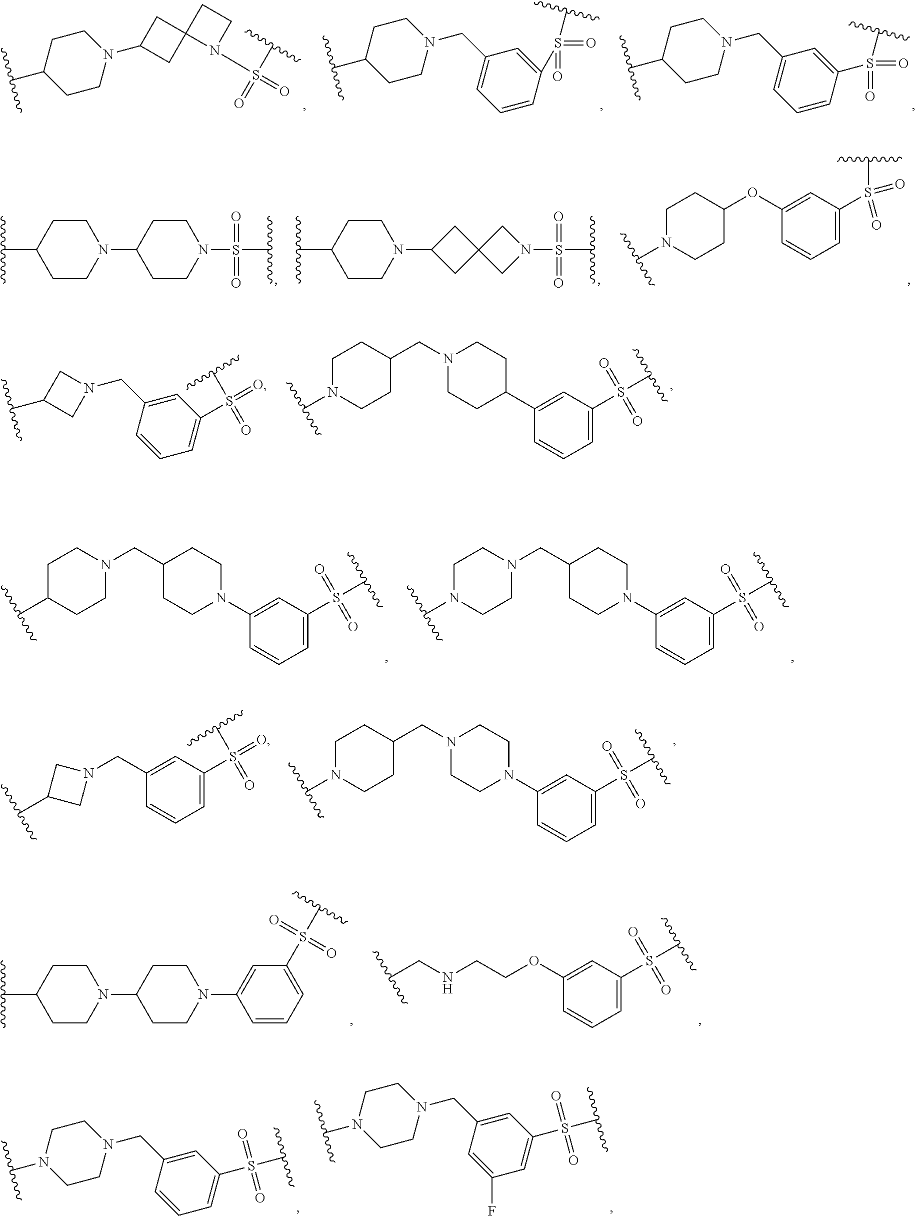

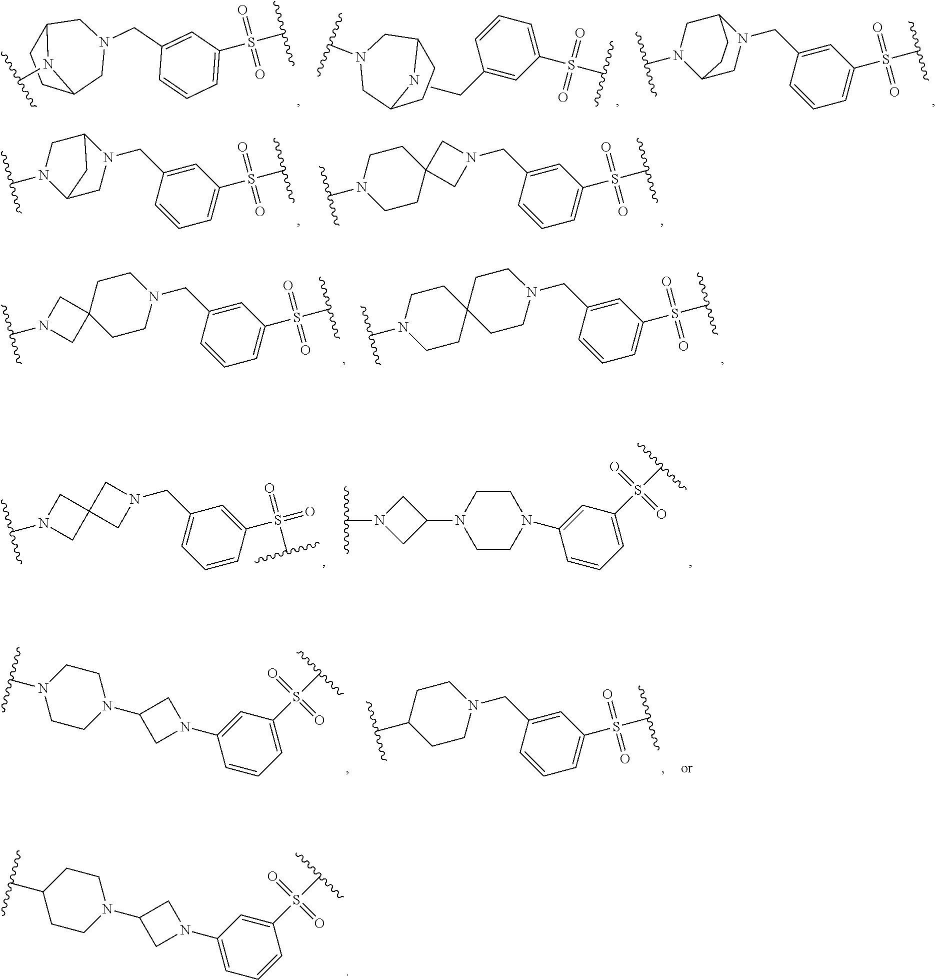

A158. In embodiment A158, the compound for use of any one of embodiments A3-1 to A96d, or a pharmaceutically acceptable salt thereof, is wherein L (when the Degron is a group of formula (iii) to (vi)), —X¹-L-, —X²-L-, —X³-L-, and —X⁴-L- (when the Degron is a group of formula (i) or (ii)) are independently:

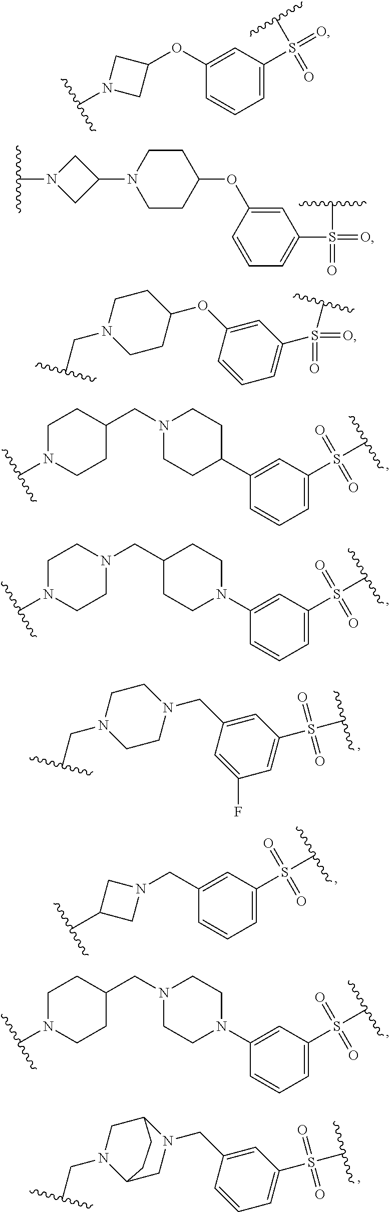

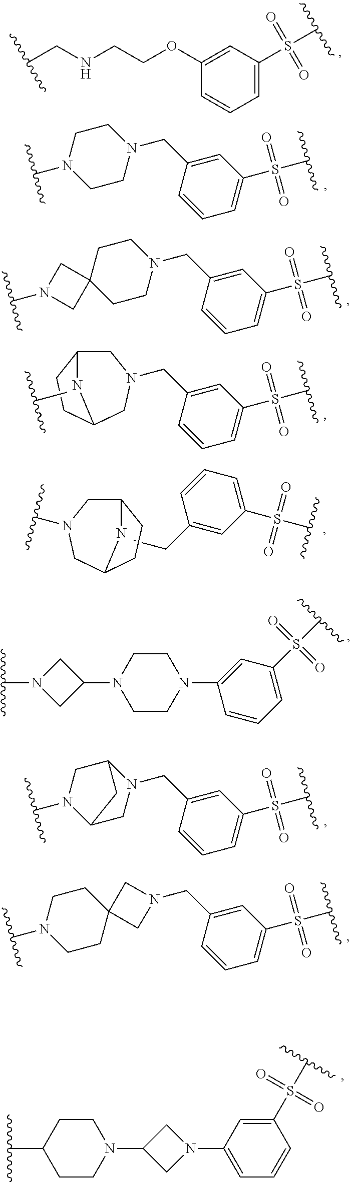

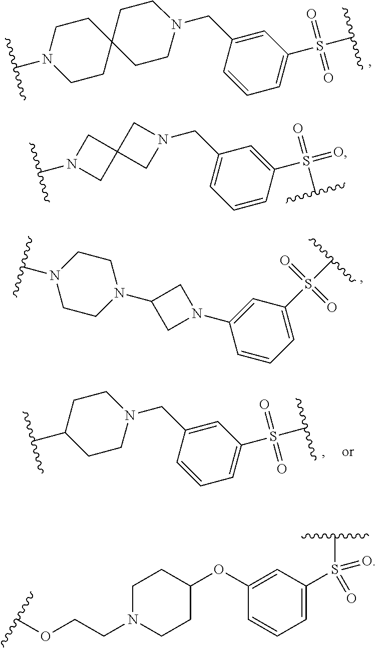

A159. In embodiment A159, the compound for use of any one of embodiments A3-1 to A110, A113, A115 to A122, A124 to A131, and A132 to A134, or a pharmaceutically acceptable salt thereof, is wherein —Z³—Z⁴—Z⁵—Z⁶— is:

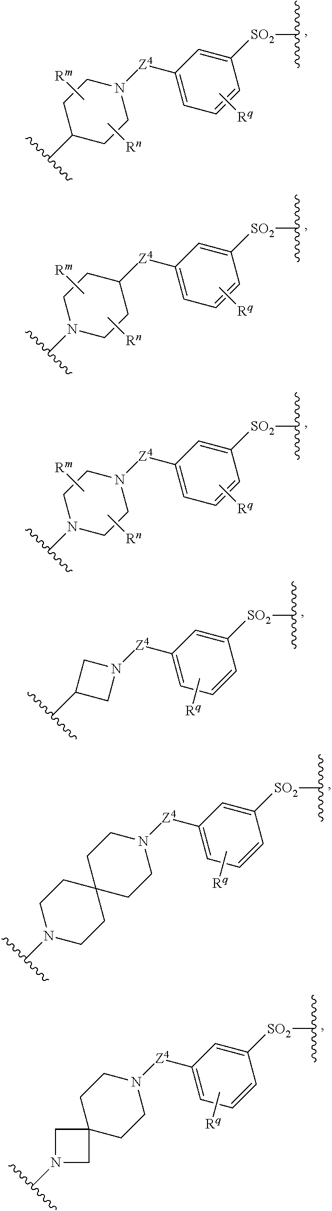

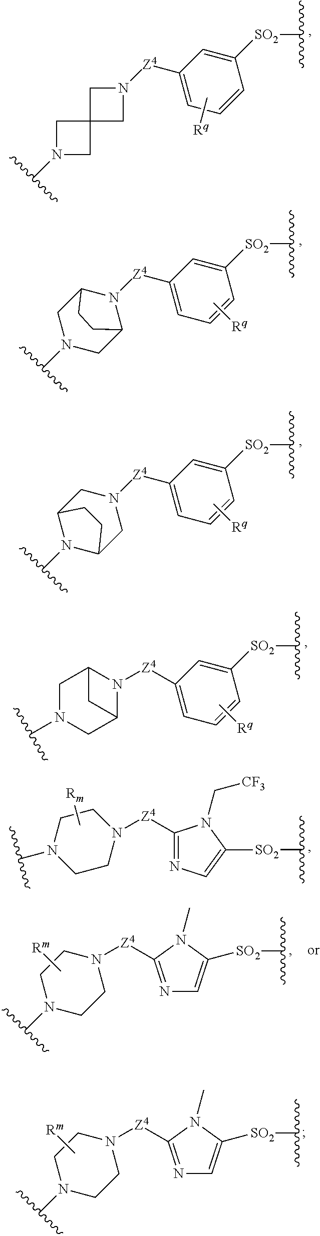

wherein each R^{m}, Rⁿ, and R^{q} are independently selected from hydrogen, alkyl, halo, haloalkyl, haloalkoxy, alkoxy, and cyano In a subembodiment, each R^{q} and R^{m} are independently selected from hydrogen, methyl, fluoro, chloro, cyano, methoxy, difluoromethoxy, difluoromethyl, and trifluoromethyl.
A160. In embodiment A160, the compound for use of any one of embodiments A3-1 to A110, A113, A115 to A122, A124 to A131, A132 to A134, and A159, or a pharmaceutically acceptable salt thereof, is wherein —Z³—Z⁴—Z⁵—Z⁶— is:

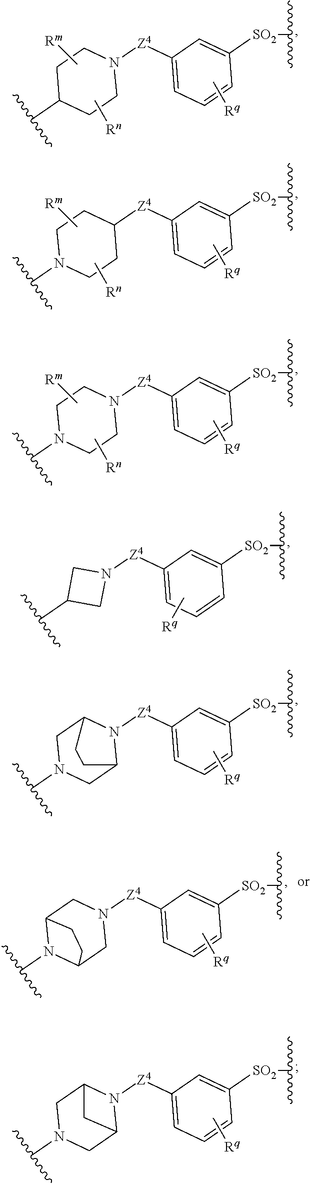

wherein each R^{m}, Rⁿ, and R^{q} are independently selected from hydrogen, alkyl, halo, haloalkyl, haloalkoxy, alkoxy, hydroxy and cyano (i.e., R^{r} is hydrogen).
A161. In embodiment A161, the compound for use of any one of embodiments A3-1 to A110, A113, A115 to A122, A124 to A131, A132 to A134, and A160, or a pharmaceutically acceptable salt thereof, is wherein —Z³—Z⁴—Z⁵—Z⁶— is:

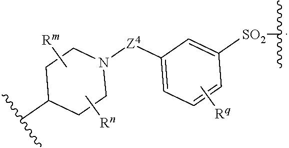

A162. In embodiment A162, the compound for use of any one of embodiments A3-1 to A110, A113, A115 to A122, A124 to A131, A132 to A134, and A160, or a pharmaceutically acceptable salt thereof, is wherein —Z³—Z⁴—Z⁵—Z⁶— is:

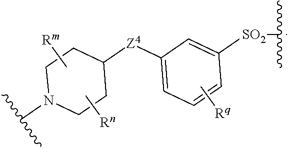

A164. In embodiment A164, the compound for use of any one of embodiments A3-1 to A110, A113, A115 to A122, A124 to A131, A132 to A134, and A160, or a pharmaceutically acceptable salt thereof, is wherein —Z³—Z⁴—Z⁵—Z⁶— is:

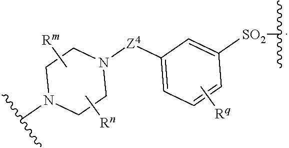

A165. In embodiment A165, the compound for use of any one of embodiments A3-1 to A110, A113, A115 to A122, A124 to A131, A132 to A134, and A160, or a pharmaceutically acceptable salt thereof, is wherein —Z³—Z⁴—Z⁵—Z⁶— is:

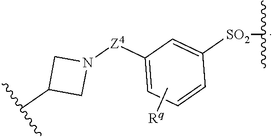

A166. In embodiment A166, the compound for use of any one of embodiments A3-1 to A110, A113, A115 to A122, A124 to A131, A132 to A134, and A160, or a pharmaceutically acceptable salt thereof, is wherein —Z³—Z⁴—Z⁵—Z⁶— is:

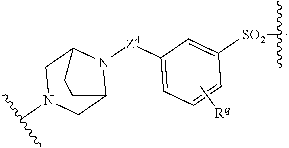

A167. In embodiment A167, the compound for use of any one of embodiments A3-1 to A110, A113, A115 to A122, A124 to A131, A132 to A134, and A160, or a pharmaceutically acceptable salt thereof, is wherein —Z³—Z⁴—Z⁵—Z⁶— is:

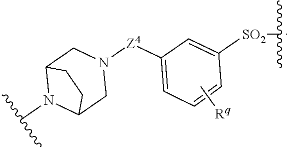

A168A. In embodiment A168A, the compound for use of any one of embodiments A3-1 to A110, A113, A115 to A122, A124 to A131, A132 to A134, and A160, or a pharmaceutically acceptable salt thereof, is wherein —Z³—Z⁴—Z³—Z⁶— is:

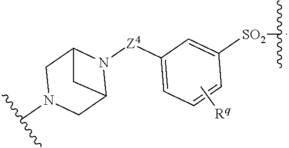

A168. In embodiment A168, the compound for use of any one of embodiments A159 to 168A, or a pharmaceutically acceptable salt thereof, is wherein

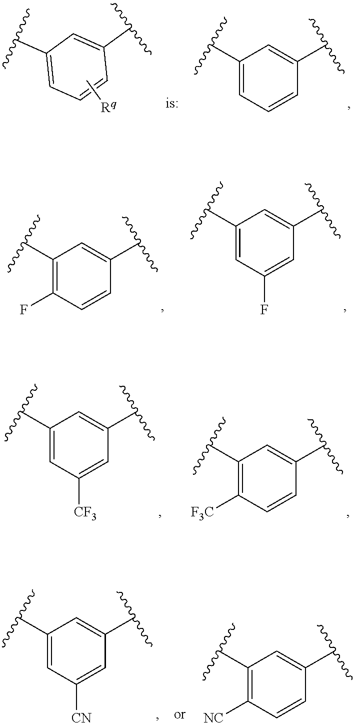

A160A. In embodiment A160A, the compound for use of any one of embodiments A3-1, A3A to A97, A111 to A113, A126, and A128 to A130A, or a pharmaceutically acceptable salt thereof, is wherein —Z³—Z⁴—Z⁵—Z⁶— is:

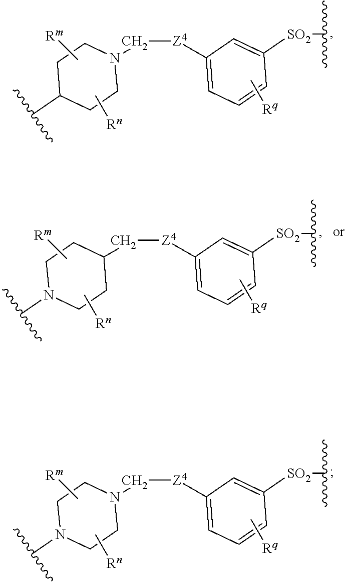

wherein each R^{m}, Rⁿ, and R^{q} are independently selected from hydrogen, alkyl, halo, haloalkyl, haloalkoxy, alkoxy, hydroxy and cyano (i.e., R^{r} is hydrogen).
A161A. In embodiment A161A, the compound for use of any one of embodiments A3-1, A3A to A97, A111 to A113, A126, A128 to A130A, and A160A, or a pharmaceutically acceptable salt thereof, is wherein —Z³—Z⁴—Z⁵—Z⁶— is:

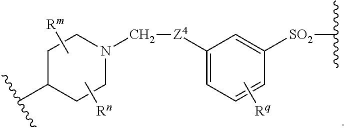

A162A. In embodiment A162A, the compound for use of any one of embodiments A3-1, A3A to A97, A111 to A113, A126, A128 to A130A, and A160A, or a pharmaceutically acceptable salt thereof, is wherein —Z³—Z⁴—Z⁵—Z⁶— is:

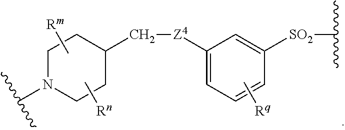

A163A. In embodiment A163A, the compound for use of any one of embodiments A3-1, A3A to A97, A111 to A113, A126, A128 to A130A, and A160A, or a pharmaceutically acceptable salt thereof, is wherein —Z³—Z⁴—Z⁵—Z⁶— is:

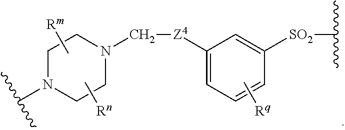

A164A. In embodiment A164A, the compound for use of any one of embodiments A3-1, A3A to A97, All to A113, A126, and A128 to A130A, or a pharmaceutically acceptable salt thereof, is wherein —Z³—Z⁴—Z⁵—Z⁶— is:

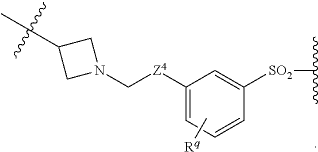

A165A. In embodiment A165A, the compound for use of any one of embodiments A160A to 165A, or a pharmaceutically acceptable salt thereof, is wherein

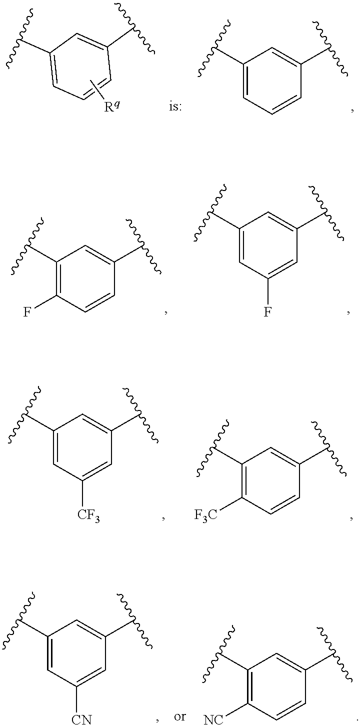

A169. In embodiment A169, the compound for use of any one of embodiments A3-1 to A121, A125 to A130, A131 to A132, A133 to A142, and A159 to A168, or a pharmaceutically acceptable salt thereof, is wherein Z⁴ is alkylene substituted with R^{s} and R^{t} where R^{s} and R^{t} are hydrogen.
A170. In embodiment A170, the compound for use of any one of embodiments A3-1 to A121, A125 to A130, A131 to A132, A133 to A142, A159 to A168, and A169, or a pharmaceutically acceptable salt thereof, is wherein the alkylene of Z⁴ is —CH₂—, —(CH₂)₂—, —(CH₂)₃—, —CH(CH₃)—, —CH₂—CH(CH₃)—CH₂—, or —CH₂—C(CH₃)₂—CH₂—.
A171. In embodiment A171, the compound for use of any one of embodiments A3-1 to A121, A125 to A130, A131 to A132, A133 to A142, A159 to A168, A169, and A170, or a pharmaceutically acceptable salt thereof, is wherein the alkylene of Z⁴ is —CH₂—.
A172. In embodiment A172, the compound for use of any one of embodiments A3-1 to A121, A125 to A130, A131 to A132, A133 to A142, and A159 to A168, or a pharmaceutically acceptable salt thereof, is wherein Z⁴ is —O—.
A173. In embodiment A173, the compound for use of any one of embodiments A3-1 to A121, A125 to A130, A131 to A132, A133 to A142, and A159 to A168, or a pharmaceutically acceptable salt thereof, is wherein Z⁴ is alkylene substituted with R^{s} and R^{t} where R^{s} is hydrogen or deuterium and R^{t} is hydrogen, deuterium, haloalkyl, hydroxy, alkoxy, or cyano.
A174. In embodiment A174, the compound for use of any one of embodiments A3-1 to A121, A125 to A130, A131 to A132, A133 to A142, A159 to A168, and A173, or a pharmaceutically acceptable salt thereof, is wherein Z⁴ is alkylene substituted with R^{s} and R^{t} where R^{s} is hydrogen or deuterium and R^{t} is hydrogen and deuterium.
A175. In embodiment A175, the compound for use of any one of embodiments A3-1 to A121, A125 to A130, A131 to A132, A133 to A142, A159 to A168, and A173, or a pharmaceutically acceptable salt thereof, is wherein Z⁴ is alkylene substituted with R^{s} and R^{t} where R^{s} is hydrogen and R^{t} is haloalkyl.
A176. In embodiment A176, the compound for use of any one of embodiments A3-1 to A121, A125 to A130, A131 to A132, A133 to A142, A159 to A168, and A173, or a pharmaceutically acceptable salt thereof, is wherein Z⁴ is alkylene substituted with R^{s} and R^{t} where R^{s} is hydrogen and R^{t} is hydroxy.
A177. In embodiment A177, the compound for use of any one of embodiments A3-1 to A121, A125 to A130, A131 to A132, A133 to A142, A159 to A168, and A173, or a pharmaceutically acceptable salt thereof, is wherein Z⁴ is alkylene substituted with R^{s} and R^{t} where R^{s} is hydrogen and R^{t} is alkoxy.
A178. In embodiment A178, the compound for use of any one of A3-1 to A121, A125 to A130, A131 to A132, A133 to A142, A159 to A168, A173, and A175 to A177, or a pharmaceutically acceptable salt thereof, is wherein Z⁴ is —CH(CHF₂)—, —CH(CF₃)—, —C(CH₃)(CF₃)—, —CH(CH₂CF₃)—, —CH(CH₂CH₂CF₃)—, —CH(CH(CF₃)₂)—, —CH(CH₂OH)—, —CH(CH₂OCH₃)—, —CH(CH₂O-ethyl)-, or —CH(CH₂CN)—, —CH₂—CH(CF₃)—CH₂—, —CH₂—CH(OH)—CH₂—, or —CH₂—CH(OCH₃)—CH₂—.
A179. In embodiment A179, the compound for use of any one of A3-1 to A114, A125 to A130, A131 to A132, A133 to A146, and A159 to A168, or a pharmaceutically acceptable salt thereof, is wherein Z⁴ is -(alkylene)-heterocyclylene-, where heterocyclylene is substituted with R^{o} and R^{p}.
A180. In embodiment A180, the compound for use of any one of A3-1 to A114, A125 to A130, A131 to A132, A133 to A146, A159 to A168, and A179 or a pharmaceutically acceptable salt thereof, is wherein Z⁴ is —(CH₂)-heterocyclylene- where heterocyclylene is substituted with R^{o} and R^{p}.
A181. In embodiment A181, the compound for use of any one of A3-1 to A114, A125 to A130, A131 to A132, A133 to A146, A159 to A168, A179, and A180 or a pharmaceutically acceptable salt thereof, is wherein Z⁴ is:

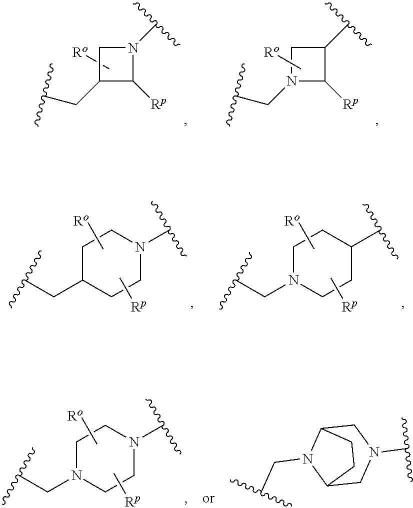

A182A. In embodiment A182A, the compound for use of any one of A3-1 to A121, A125 to A130, A131, A132, A133 to A137b, A159 to A168, A160A to A165A, and A179 to A181 or a pharmaceutically acceptable salt thereof, is wherein —Z³—Z⁴—Z⁵—Z⁶— is:

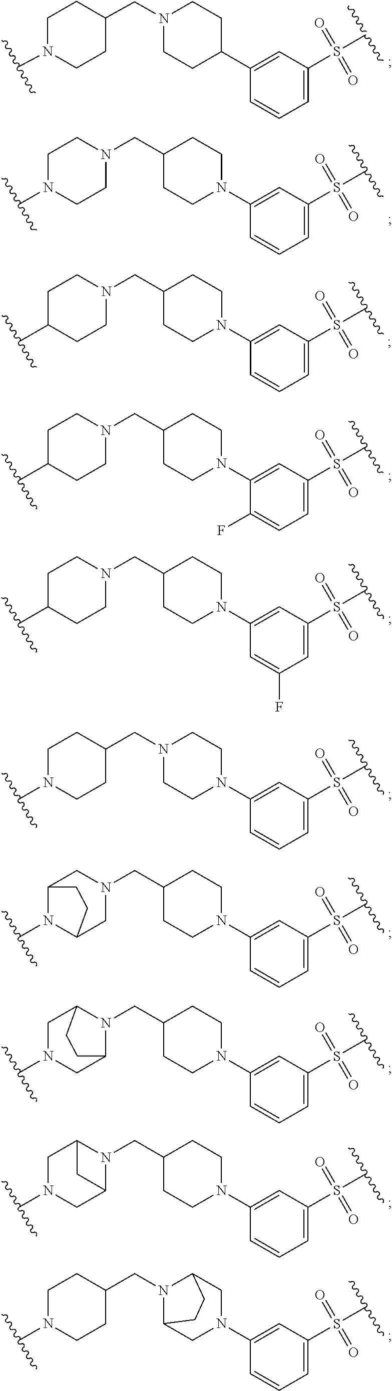

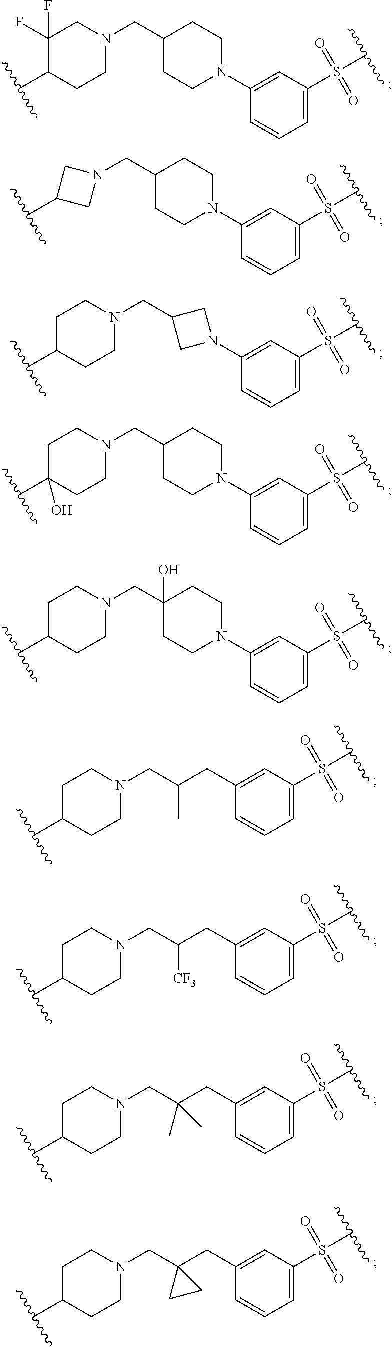

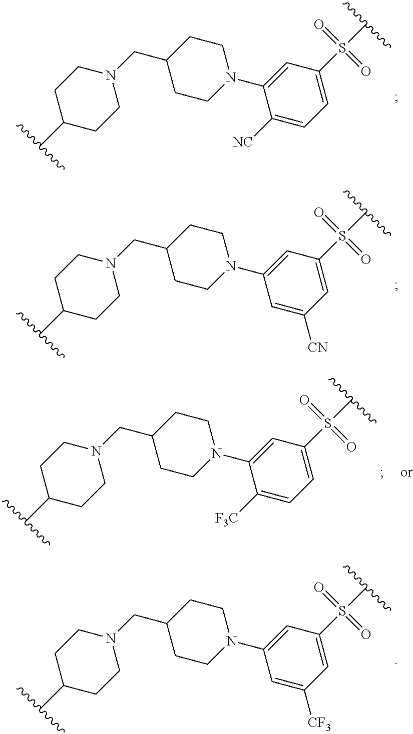 [SMILES: *C1CCN(CC2CCN(c3cc(S(*)(=O)=O)ccc3C#N)CC2)CC1 ; *C1CCN(CC2CCN(c3cc(C#N)cc(S(*)(=O)=O)c3)CC2)CC1 ; *C1CCN(CC2CCN(c3cc(S(*)(=O)=O)ccc3C(F)(F)F)CC2)CC1 ; *C1CCN(CC2CCN(c3cc(C(F)(F)F)cc(S(*)(=O)=O)c3)CC2)CC1]

A182. In embodiment A182, the compound for use of any one of A3-1 to A121, A125 to A130, A131, A132, A133 to A137b, A159 to A168, A160A to A165A, and A179 to A182A, or a pharmaceutically acceptable salt thereof, is wherein —Z³—Z⁴—Z³—Z⁶— is:

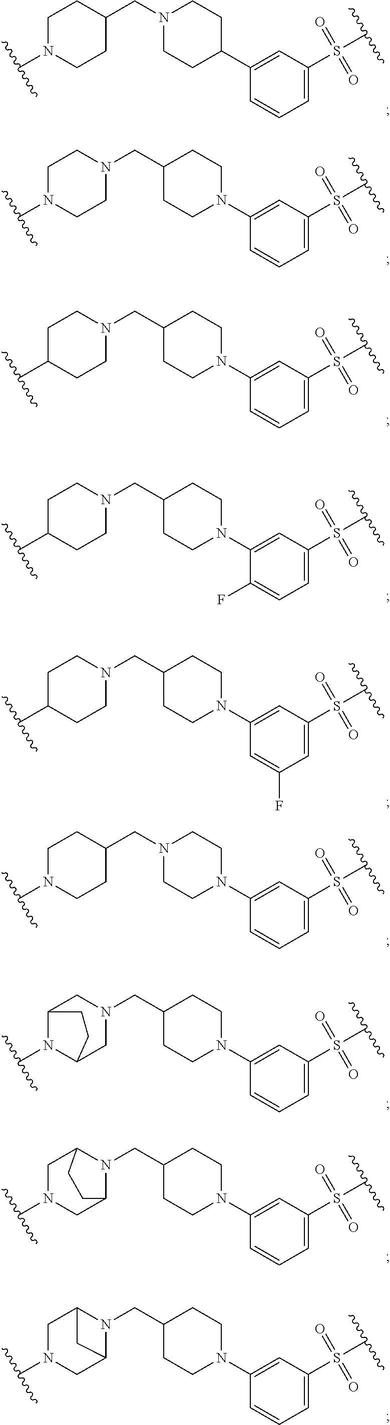

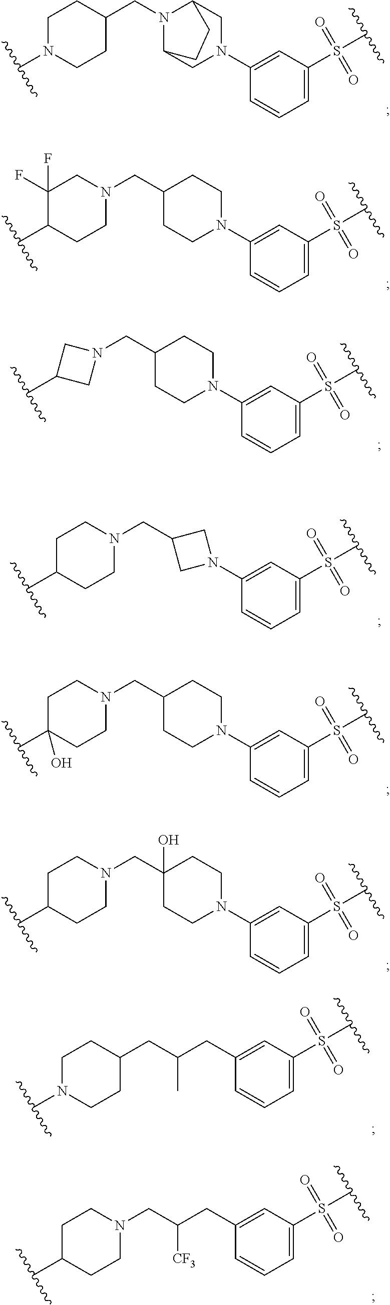

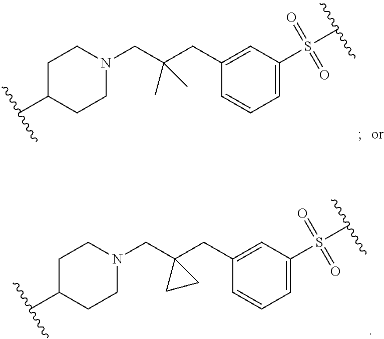 [SMILES: *C1CCN(CC(C)(C)Cc2cccc(S(*)(=O)=O)c2)CC1 ; *C1CCN(CC2(Cc3cccc(S(*)(=O)=O)c3)CC2)CC1]

A183A. In embodiment A183A, the compound for use of any one of A3-1 to A121, A125 to A130, A131, A132, A133 to A137b, A159 to A168, A160A to A165A, and A179 to A182A, or a pharmaceutically acceptable salt thereof, is wherein —Z³—Z⁴—Z⁵—Z⁶— is:

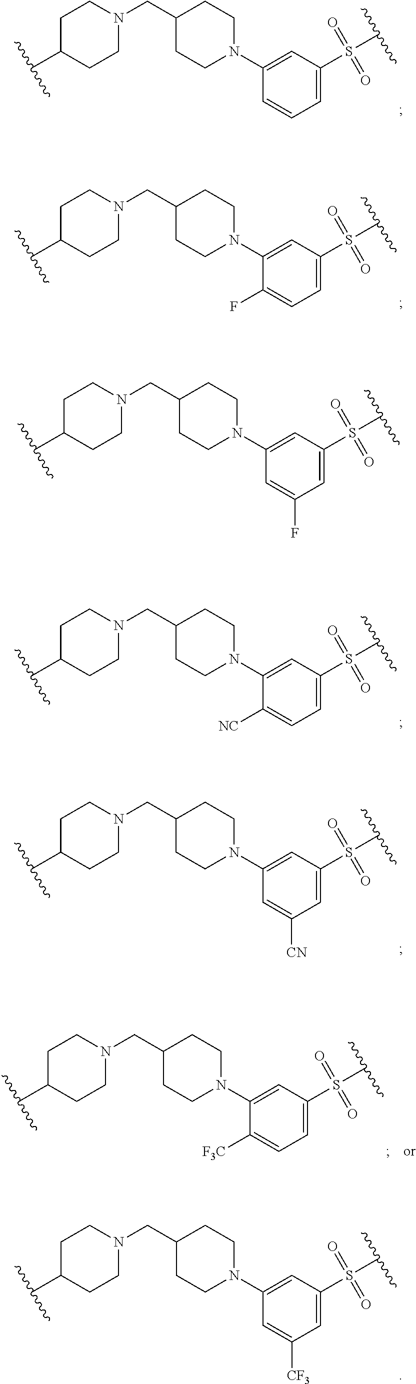

A183. In embodiment A183, the compound for use of any one of A3-1 to A121, A125 to A130, A131, A132, A133 to A137b, A159 to A168, A160A to A165A, and A179 to A183A, or a pharmaceutically acceptable salt thereof, is wherein —Z³—Z⁴—Z⁵—Z⁶— is:

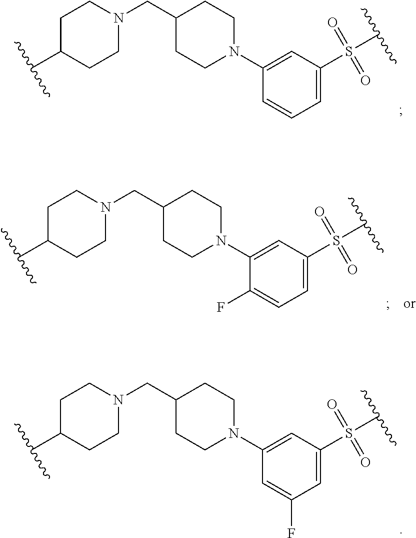 [SMILES: *C1CCN(CC2CCN(c3cccc(S(*)(=O)=O)c3)CC2)CC1 ; *C1CCN(CC2CCN(c3cc(S(*)(=O)=O)ccc3F)CC2)CC1 ; *C1CCN(CC2CCN(c3cc(F)cc(S(*)(=O)=O)c3)CC2)CC1]

A184. In embodiment A184, the compound for use of any one of A3-1 to A96d, or a pharmaceutically acceptable salt thereof, is wherein L (when the Degron is a group of formula (iii) to (vi)), —X¹-L-, —X²-L-, —X³-L- and —X⁴-L- (when the Degron is a group of formula (i) or (ii)) are independently:

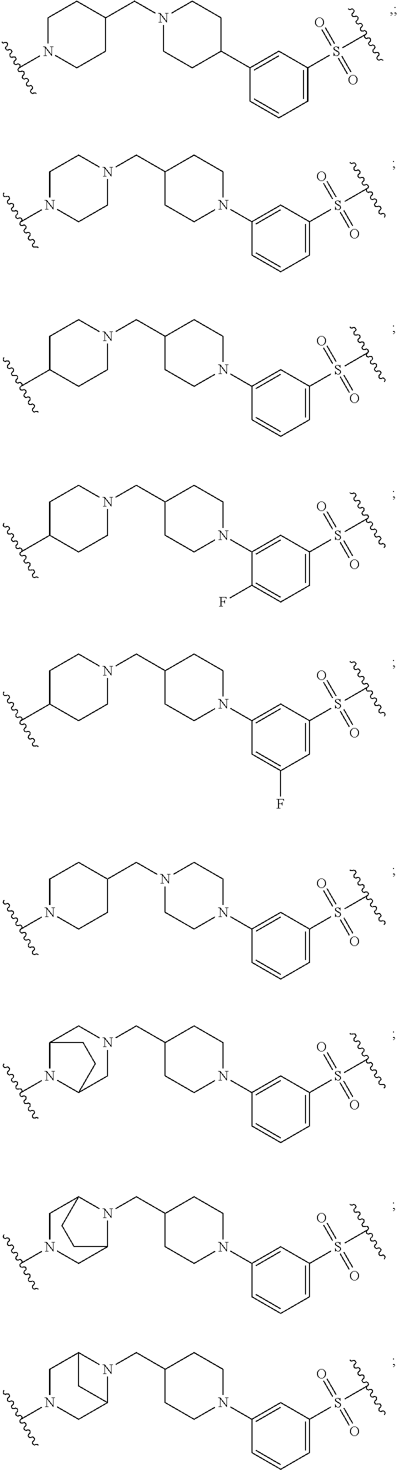

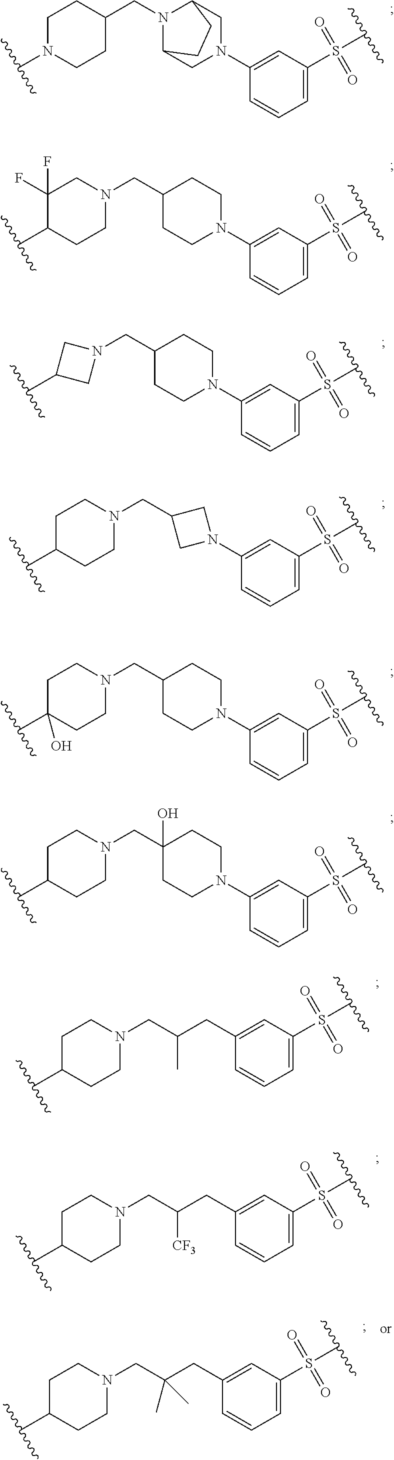

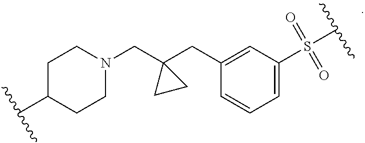 [SMILES: *C1CCN(CC2(Cc3cccc(S(*)(=O)=O)c3)CC2)CC1]

A185. In embodiment A185, the compound for use of any one of A3-1 to A96d and A184, or a pharmaceutically acceptable salt thereof, is wherein L (when the Degron is a group of formula (iii) to (vi)), —X¹-L-, —X²-L-, —X³-L- and —X⁴-L- (when the Degron is a group of formula (i) or (ii)) are independently:

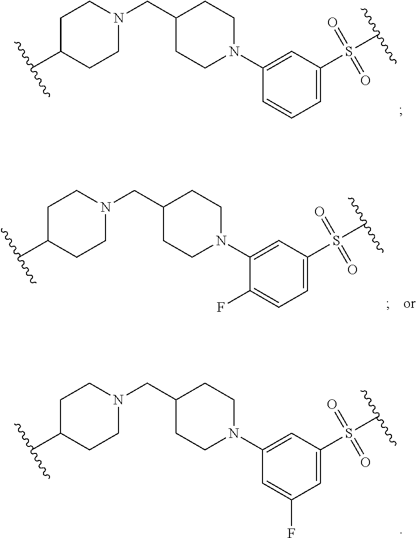 [SMILES: *C1CCN(CC2CCN(c3cccc(S(*)(=O)=O)c3)CC2)CC1 ; *C1CCN(CC2CCN(c3cc(S(*)(=O)=O)ccc3F)CC2)CC1 ; *C1CCN(CC2CCN(c3cc(F)cc(S(*)(=O)=O)c3)CC2)CC1]

A186A. In embodiment A186A, the compound for use of any one of embodiments A3-1 to A96 and A97 to A185, or a pharmaceutically acceptable salt thereof, is wherein Degron is the E3 ubiquitin ligase ligand selected from:

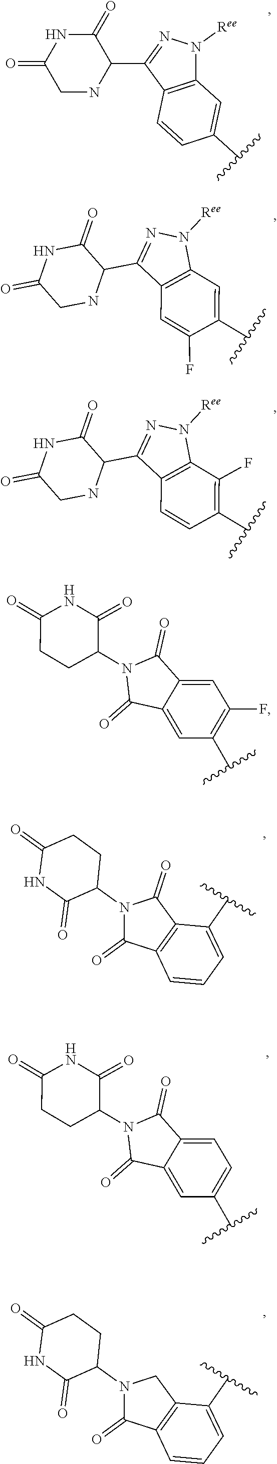

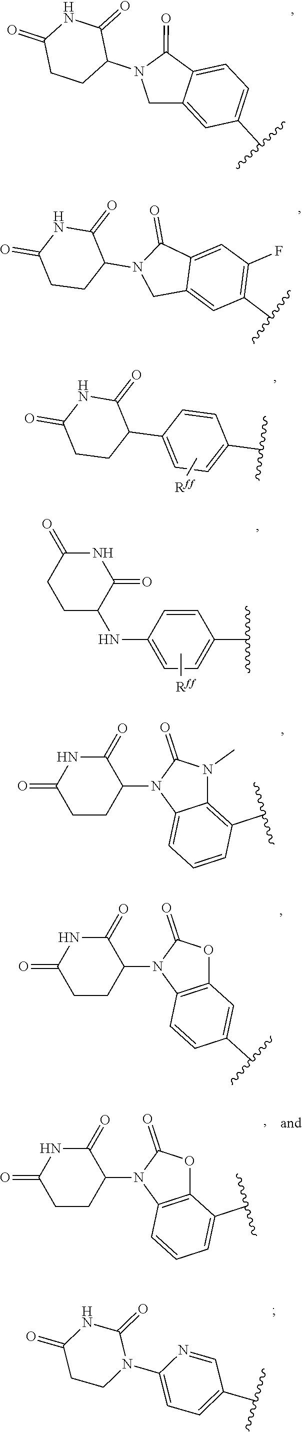

where R^{ee} is hydrogen, methyl, ethyl, cyclopropyl, or 2,2,2-trifluoroethyl and R^{ff} is hydrogen, methyl, cyclopropyl, fluoro, cyano, methoxy, difluoromethoxy, trifluoromethoxy, or trifluoromethyl.
A186. In embodiment A186, the compound for use of any one of embodiments A3-1 to A96, and A97 to A186A, or a pharmaceutically acceptable salt thereof, is wherein Degron is the E3 ubiquitin ligase ligand selected from:

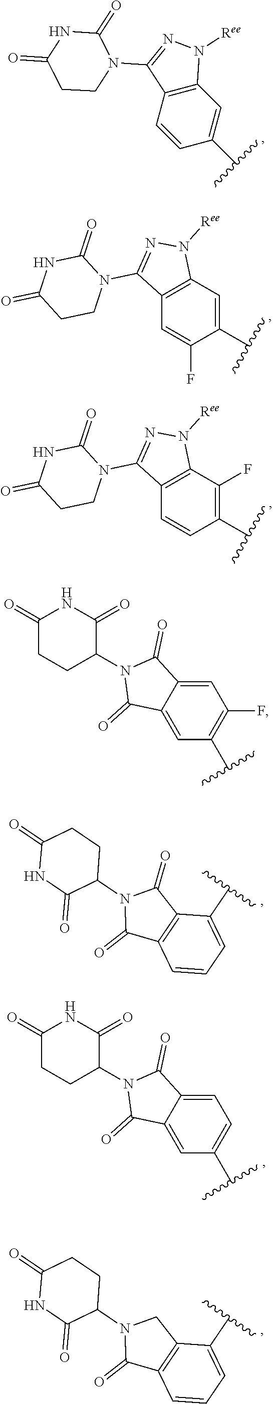

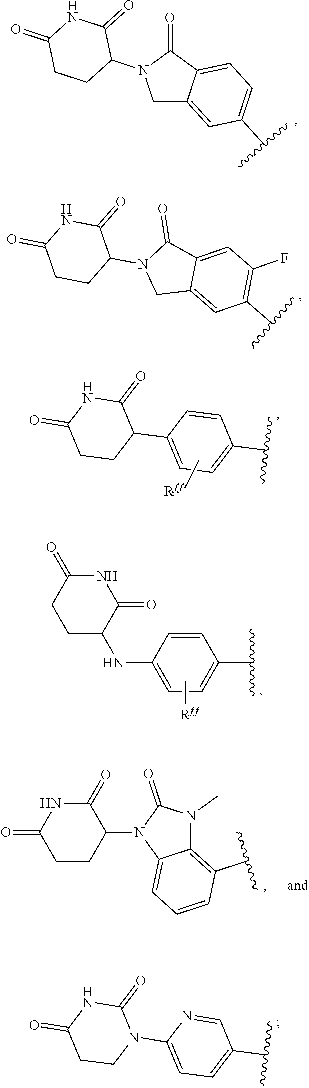

where R^{ee} is hydrogen, methyl, ethyl, cyclopropyl, or 2,2,2-trifluoroethyl and R^{ff} is hydrogen, methyl, cyclopropyl, fluoro, cyano, methoxy, difluoromethoxy, trifluoromethoxy, or trifluoromethyl.
A187. In embodiment A187, the compound for use of any one of embodiments A3-1 to A96 and A97 to A186, or a pharmaceutically acceptable salt thereof, is wherein Degron is the E3 ligase ligand selected from:

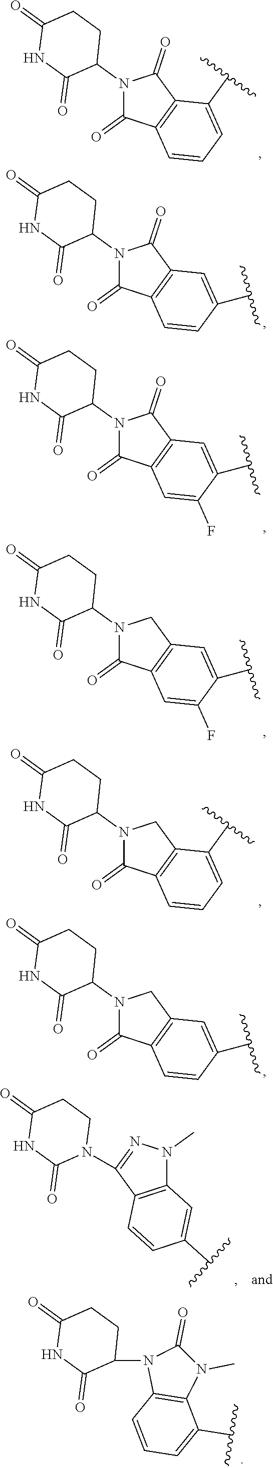

A188. In embodiment A188, the compound for use of any one of embodiments A3-1 to A96 and A97 to A186, or a pharmaceutically acceptable salt thereof, is wherein Degron is the E3 ubiquitin ligase ligand is

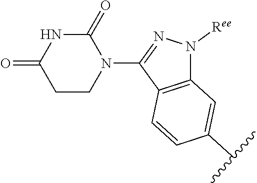

where each R^{ee} is hydrogen, methyl, ethyl, cyclopropyl, or 2,2,2-trifluoroethyl, preferably methyl and each R^{ff}, when present, is hydrogen, methyl, cyclopropyl, fluoro, cyano, methoxy, difluoromethoxy, trifluoromethoxy, or trifluoromethyl.
A189. In embodiment A189, the compound for use of any one of embodiments A3-1 to A96 and A97 to A186, or a pharmaceutically acceptable salt thereof, is wherein Degron is the E3 ubiquitin ligase ligand is

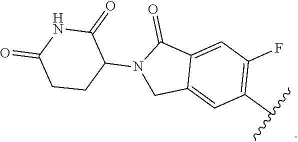 [SMILES: *c1cc2c(cc1F)C(=O)N(C1CCC(=O)NC1=O)C2]

A190. In embodiment A190, the compound for use of any one of embodiments A3-1 to A185, or a pharmaceutically acceptable salt thereof, is wherein R^{x} and R^{x1} are hydrogen.
For sake of clarity, embodiment A includes combination of embodiments A and subembodiments thereof.
For sake of clarity, when an embodiment refers to more than one preceding embodiments of varying scopes, the scope of the preceding embodiments control i.e., only those groups that fall within the scope of group(s) recited in a preceding embodiment(s) should be selected from the embodiment referring thereto. For example, of the groups recited in embodiment A8, while all the recited groups in A8 should be selected for embodiment A1, only fluoro, chloro, and bromo should be selected for embodiment A5 as scope of R¹ in A5 is limited to halo; and only difluoromethyl, trifluoromethyl, difluoroethyl, and trifluoroethyl should be selected for embodiment A6 as scope of R¹ in A6 is limited to haloalkyl.

Additional embodiments B1-157 are:
B1A. In embodiment B1A, the compound for use of the first aspect, or a pharmaceutically acceptable salt thereof, is wherein the compound (or any one of the embodiments thereof disclosed in the Summary) further comprises a linker attached to the Hy of CDK2 binding moiety of formula (A1).
B1. In embodiment B1, the compound for use of the second aspect, or a pharmaceutically acceptable salt thereof, is wherein the compound further comprises a linker attached to the Hy of CDK2 binding moiety of formula (A).
B2-1 to B2-40. In embodiments B2-1 to B2-40, the compound for use of embodiment B1 is wherein the moiety of formula (A) is as disclosed in embodiments A4-50 and A4 to A39b respectively, or a pharmaceutically acceptable salt thereof.
B2-41 to B2-112. In embodiments B2-41 to B2-110, the compound for use of embodiment B1A is wherein the moiety of formula (A1) is as disclosed in embodiments A4-1 to A4-50, A4, and A23 to A39b respectively, or a pharmaceutically acceptable salt thereof.
B3. In embodiment B3, the compound for use of any one of embodiments B1A, B1, and B2-1 to B2-112, or a pharmaceutically acceptable salt thereof, is wherein the compound further comprises an E3 ubiquitin ligase ligand and wherein the ligand is attached to the linker and the compound is a compound of Formula (II):

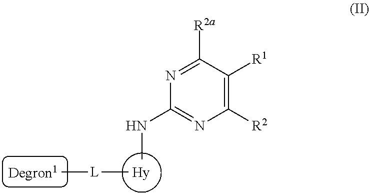

where Degron¹ is an E3 ubiquitin ligase ligand.
B4. In embodiment B4, the compound for use of any one of embodiments B1A to B3, or a pharmaceutically acceptable salt thereof, is wherein the linker comprises one or more —SO₂—.
B5. In embodiment B5, the compound for use of any one of embodiments B1A to B4, or a pharmaceutically acceptable salt thereof, is wherein the linker comprises one or more —SO₂— and one of the one or more —SO₂— is attached to Hy of formula (A1), (A), and (II). In a subembodiment of embodiment B5, the linker comprises one or more —SO₂— and one of the one or more —SO₂— is attached to a nitrogen in Hy of formula (A1), (A), and (II).
B6. In embodiment B6, the compound for use of any one of embodiments and B1A to
B5, or a pharmaceutically acceptable salt thereof, is wherein the linker comprises one or two —SO₂—.
B7 In embodiment B7, the compound for use of any one of embodiments and B1A to B6, or a pharmaceutically acceptable salt thereof, is wherein the linker comprises one —SO₂—.
B8. In embodiment B8, the compound for use of any one of embodiments B1A to B3, or a pharmaceutically acceptable salt thereof, is wherein the linker comprises one or more rings independently selected from cycloalkylene, bridged cycloalkylene, phenylene, heteroarylene, heterocyclylene, bridged heterocyclylene, and spiro heterocyclylene, where each ring is optionally substituted with one or two substituents independently selected from deuterium, alkyl, alkoxy, halo, haloalkyl, haloalkoxy, cyano, hydroxy, amino, alkylamino, and dialkylamino.
B8a. In embodiment B8a, the compound for use of any one of embodiments BIA to B3, or a pharmaceutically acceptable salt thereof, is wherein the linker comprises one or more rings independently selected from cycloalkylene, bridged cycloalkylene, phenylene, heteroarylene, heterocyclylene, bridged heterocyclylene, and spiro heterocyclylene, where each ring is optionally substituted with one or two substituents independently selected from deuterium, alkyl, alkoxy, halo, haloalkyl, haloalkoxy, cyano, hydroxy, carboxy, alkoxycarbonyl, amino, alkylamino, and dialkylamino.
B9. In embodiment B9, the compound for use of any one of embodiments B1A to B3 and B8, or a pharmaceutically acceptable salt thereof, is wherein the linker comprises one to four rings independently selected from cycloalkylene, bridged cycloalkylene, phenylene, heteroarylene, heterocyclylene, bridged heterocyclylene, and spiro heterocyclylene, where each ring is optionally substituted with one or two substituents independently selected from deuterium, alkyl, alkoxy, halo, haloalkyl, haloalkoxy, cyano, hydroxy, amino, alkylamino, and dialkylamino.
B9a. In embodiment B9a, the compound for use of any one of embodiments B1A to B3 and B8a, or a pharmaceutically acceptable salt thereof, is wherein the linker comprises one to four rings independently selected from cycloalkylene, bridged cycloalkylene, phenylene, heteroarylene, heterocyclylene, bridged heterocyclylene, and spiro heterocyclylene, where each ring is optionally substituted with one or two substituents independently selected from deuterium, alkyl, alkoxy, halo, haloalkyl, haloalkoxy, cyano, hydroxy, carboxy, alkoxycarbonyl, amino, alkylamino, and dialkylamino.
B10. In embodiment B10, the compound for use of any one of embodiments BIA to B3, B8, and B9, or a pharmaceutically acceptable salt thereof, is wherein the linker comprises one or two rings independently selected from cycloalkylene, bridged cycloalkylene, phenylene, heteroarylene, heterocyclylene, bridged heterocyclylene, and spiro heterocyclylene, where each ring is optionally substituted with one or two substituents independently selected from deuterium, alkyl, alkoxy, halo, haloalkyl, haloalkoxy, cyano, hydroxy, amino, alkylamino, and dialkylamino. In a subembodiment of B10, the linker comprises one ring. In a second subembodiment of B10, the linker comprises two rings.
B10a. In embodiment B10a, the compound for use of any one of embodiments BIA to B3, B8a, and B9a, or a pharmaceutically acceptable salt thereof, is wherein the linker comprises one or two rings independently selected from cycloalkylene, bridged cycloalkylene, phenylene, heteroarylene, heterocyclylene, bridged heterocyclylene, and spiro heterocyclylene, where each ring is optionally substituted with one or two substituents independently selected from deuterium, alkyl, alkoxy, halo, haloalkyl, haloalkoxy, cyano, hydroxy, carboxy, alkoxycarbonyl, amino, alkylamino, and dialkylamino. In a subembodiment of B10a, the linker comprises one ring. In a second subembodiment of B10a, the linker comprises two rings.
B11. In embodiment B11, the compound for use of any one of embodiments B1A to B3, B8, and B9, or a pharmaceutically acceptable salt thereof, is wherein the linker comprises three rings independently selected from cycloalkylene, bridged cycloalkylene, phenylene, heteroarylene, heterocyclylene, bridged heterocyclylene, and spiro heterocyclylene, where each ring is optionally substituted with one or two substituents independently selected from deuterium, alkyl, alkoxy, halo, haloalkyl, haloalkoxy, cyano, hydroxy, amino, alkylamino, and dialkylamino.
B11a. In embodiment B11a, the compound for use of any one of embodiments B1A to B3, B8a, and B9a, or a pharmaceutically acceptable salt thereof, is wherein the linker comprises three rings independently selected from cycloalkylene, bridged cycloalkylene, phenylene, heteroarylene, heterocyclylene, bridged heterocyclylene, and spiro heterocyclylene, where each ring is optionally substituted with one or two substituents independently selected from deuterium, alkyl, alkoxy, halo, haloalkyl, haloalkoxy, cyano, hydroxy, carboxy, alkoxycarbonyl, amino, alkylamino, and dialkylamino.
B12. In embodiment B12, the compound for use of any one of embodiments B4 to B7, or a pharmaceutically acceptable salt thereof, is wherein the linker further comprises one or more rings independently selected from cycloalkylene, bridged cycloalkylene, phenylene, heteroarylene, heterocyclylene, bridged heterocyclylene, and spiro heterocyclylene, where each ring is optionally substituted with one or two substituents independently selected from deuterium, alkyl, alkoxy, halo, haloalkyl, haloalkoxy, cyano, hydroxy, amino, alkylamino, and dialkylamino.
B12a. In embodiment B12a, the compound for use of any one of embodiments B4 to B7, or a pharmaceutically acceptable salt thereof, is wherein the linker further comprises one or more rings independently selected from cycloalkylene, bridged cycloalkylene, phenylene, heteroarylene, heterocyclylene, bridged heterocyclylene, and spiro heterocyclylene, where each ring is optionally substituted with one or two substituents independently selected from deuterium, alkyl, alkoxy, halo, haloalkyl, haloalkoxy, cyano, hydroxy, carboxy, alkoxycarbonyl, amino, alkylamino, and dialkylamino.
B13. In embodiment B13, the compound for use of any one of embodiments B4 to B7 and B12, or a pharmaceutically acceptable salt thereof, is wherein the linker further comprises one to four rings independently selected from cycloalkylene, bridged cycloalkylene, phenylene, heteroarylene, heterocyclylene, bridged heterocyclylene, and spiro heterocyclylene, where each ring is optionally substituted with one or two substituents independently selected from deuterium, alkyl, alkoxy, halo, haloalkyl, haloalkoxy, cyano, hydroxy, amino, alkylamino, and dialkylamino.
B13a. In embodiment B13a, the compound for use of any one of embodiments B4 to B7 and B12a, or a pharmaceutically acceptable salt thereof, is wherein the linker further comprises one to four rings independently selected from cycloalkylene, bridged cycloalkylene, phenylene, heteroarylene, heterocyclylene, bridged heterocyclylene, and spiro heterocyclylene, where each ring is optionally substituted with one or two substituents independently selected from deuterium, alkyl, alkoxy, halo, haloalkyl, haloalkoxy, cyano, hydroxy, carboxy, alkoxycarbonyl, amino, alkylamino, and dialkylamino.
B14. In embodiment B14, the compound for use of any one of embodiments B4 to B7 and B12, or a pharmaceutically acceptable salt thereof, is wherein the linker further comprises one or two rings independently selected from cycloalkylene, bridged cycloalkylene, phenylene, heteroarylene, heterocyclylene, bridged heterocyclylene, and spiro heterocyclylene, where each ring is optionally substituted with one or two substituents independently selected from deuterium, alkyl, alkoxy, halo, haloalkyl, haloalkoxy, cyano, hydroxy, amino, alkylamino, and dialkylamino.
B14a. In embodiment B14a, the compound for use of any one of embodiments B4 to B7 and B12a, or a pharmaceutically acceptable salt thereof, is wherein the linker further comprises one or two rings independently selected from cycloalkylene, bridged cycloalkylene, phenylene, heteroarylene, heterocyclylene, bridged heterocyclylene, and spiro heterocyclylene, where each ring is optionally substituted with one or two substituents independently selected from deuterium, alkyl, alkoxy, halo, haloalkyl, haloalkoxy, cyano, hydroxy, carboxy, alkoxycarbonyl, amino, alkylamino, and dialkylamino.
B15. In embodiment B15, the compound for use of any one of embodiments B4 to B7 and B12, or a pharmaceutically acceptable salt thereof, is wherein the linker further comprises three rings independently selected from cycloalkylene, bridged cycloalkylene, phenylene, heteroarylene, heterocyclylene, bridged heterocyclylene, and spiro heterocyclylene, where each ring is optionally substituted with one or two substituents independently selected from deuterium, alkyl, alkoxy, halo, haloalkyl, haloalkoxy, cyano, hydroxy, amino, alkylamino, and dialkylamino.
B15a. In embodiment B15a, the compound for use of any one of embodiments B4 to B7 and B12a, or a pharmaceutically acceptable salt thereof, is wherein the linker further comprises three rings independently selected from cycloalkylene, bridged cycloalkylene, phenylene, heteroarylene, heterocyclylene, bridged heterocyclylene, and spiro heterocyclylene, where each ring is optionally substituted with one or two substituents independently selected from deuterium, alkyl, alkoxy, halo, haloalkyl, haloalkoxy, cyano, hydroxy, carboxy, alkoxycarbonyl, amino, alkylamino, and dialkylamino.
B16. In embodiment B16, the compound for use of any one of embodiments B8 to B15a, or a pharmaceutically acceptable salt thereof, is wherein the rings are independently selected from phenylene, heteroarylene, heterocyclylene, bridged heterocyclylene, and spiro heterocyclylene, where each ring is optionally substituted as stated therein.
B17. In embodiment B17, the compound for use of any one of embodiments B12 to B16, or a pharmaceutically acceptable salt thereof, is wherein one of the rings is attached to the —SO₂— that is attached to Hy of formula A or A1. In a subembodiment of embodiment B17, one of the rings is attached to the —SO₂—, Hy contains a substitutable nitrogen atom, the —SO₂— is attached to the substitutable nitrogen of Hy of formula A or A1.
B18. In embodiment B18, the compound for use of any one of embodiments B12 to B17, or a pharmaceutically acceptable salt thereof, is wherein a phenylene is attached to the —SO₂— that is attached to Hy of formula A or A1 and is optionally substituted as stated therein. In a subembodiment of embodiment B18, phenylene is attached to the —SO₂—, Hy contains a substitutable nitrogen atom, the —SO₂— is attached to the substitutable nitrogen of Hy of formula A or A1.
B19. In embodiment B19, the compound for use of any one of embodiments B12 to B17, or a pharmaceutically acceptable salt thereof, is wherein a heteroarylene is attached to the —SO₂— that is attached to Hy of formula A or A1 and is optionally substituted as stated therein. In a subembodiment of embodiment B19, heteroarylene is attached to the —SO₂—, Hy contains a substitutable nitrogen atom, the —SO₂— is attached to the substitutable nitrogen of Hy of formula A or A1. B20. In embodiment B20, the compound for use of any one of embodiments B12 to B17, or a pharmaceutically acceptable salt thereof, is wherein a heterocyclylene is attached to the —SO₂— that is attached to Hy of formula A or A1 and is optionally substituted as stated therein. In a subembodiment of embodiment B20, heterocyclylene is attached to the —SO₂—, Hy contains a substitutable nitrogen atom, the —SO₂— is attached to the substitutable nitrogen of Hy of formula A or A1.
B21. In embodiment B21, the compound for use of any one of embodiments B12 to B17, or a pharmaceutically acceptable salt thereof, is wherein a bridged heterocyclylene is attached to the —SO₂— that is attached to Hy of formula A or A1 and is optionally substituted as stated therein. In a subembodiment of embodiment B21, bridged heterocyclylene is attached to the —SO₂—, Hy contains a substitutable nitrogen atom, the —SO₂— is attached to the substitutable nitrogen of Hy of formula A or A1.
B22. In embodiment B22, the compound for use of any one of embodiments B12 to B17, or a pharmaceutically acceptable salt thereof, is wherein a spiro heterocyclylene is attached to the —SO₂— attached to Hy of formula A or A1 and is optionally substituted as stated therein. In a subembodiment of embodiment B22, spiro heterocyclylene is attached to the —SO₂—, Hy contains a substitutable nitrogen atom, the —SO₂— is attached to the substitutable nitrogen of Hy of formula A or A1.
B23. In embodiment B23, the compound for use of any one of embodiments B8 to B22, or a pharmaceutically acceptable salt thereof, is wherein two of said rings are adjacent to each other.
B23a. In embodiment B23a, the compound for use of embodiment B23, or a pharmaceutically acceptable salt thereof, is wherein the second ring is adjacent to the ring attached to —SO₂—.
B23b. In embodiment B23b, the compound for use of embodiment B23a, or a pharmaceutically acceptable salt thereof, is wherein the ring adjacent to the ring attached to —SO₂— is selected from heterocyclylene, bridged heterocyclylene, and spiro heterocyclylene, where each ring is optionally substituted as stated therein.
B24. In embodiment B24, the compound for use of any one of embodiments B8 to B23b, or a pharmaceutically acceptable salt thereof, is wherein each of the cycloalkylene, bridged cycloalkylene, phenylene, heteroarylene, heterocyclylene, bridged heterocyclylene, and spiro heterocyclylene is independently selected from:

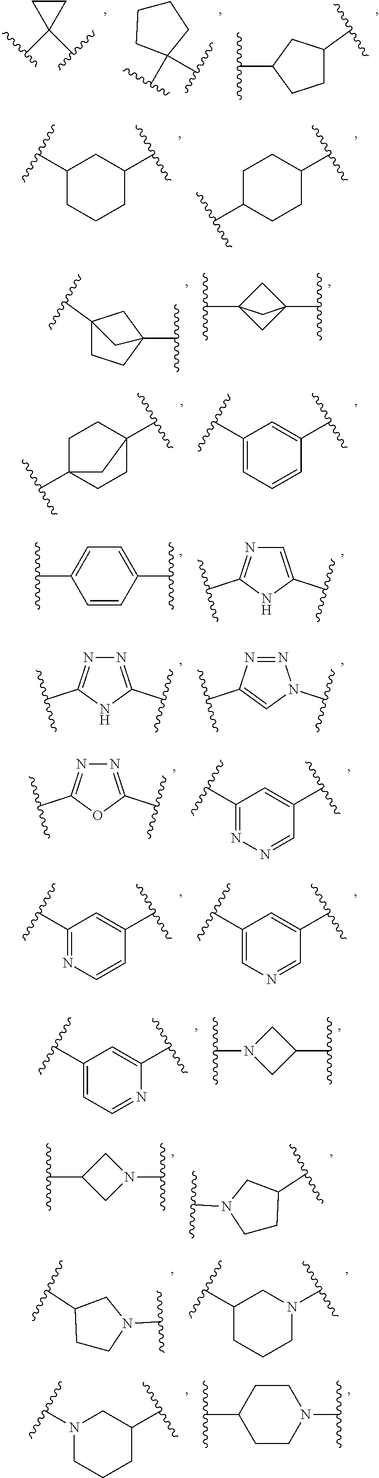

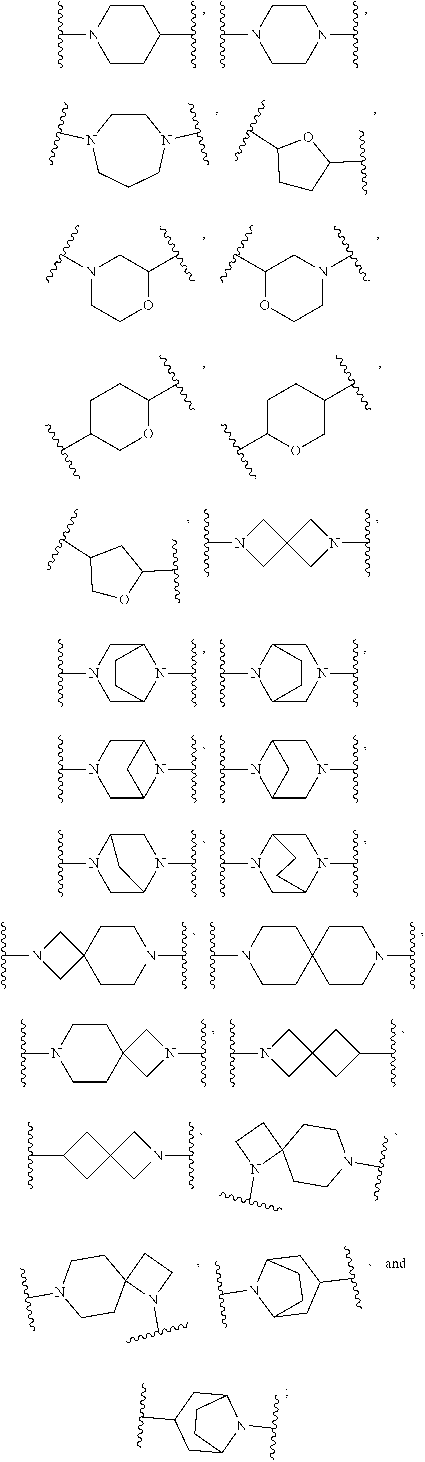

where each ring is substituted as defined therein.
B25. In embodiment B25, the compound for use of any one of embodiments B8 to B24, or a pharmaceutically acceptable salt thereof, is wherein the rings stated therein are independently selected from phenylene, heteroarylene, heterocyclylene, bridged heterocyclylene, and spiro heterocyclylene and each of the phenylene, heteroarylene, heterocyclylene, bridged heterocyclylene, and spiro heterocyclylene is independently selected from:

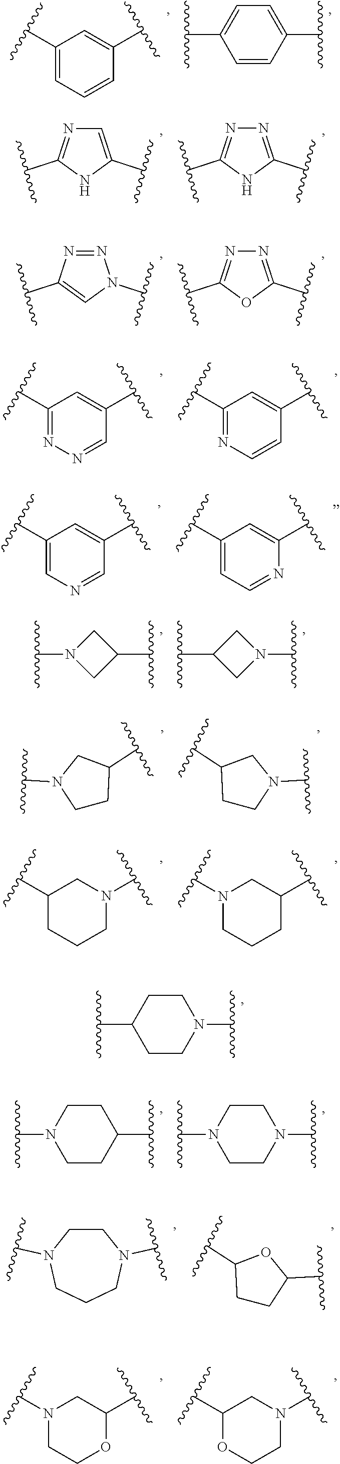

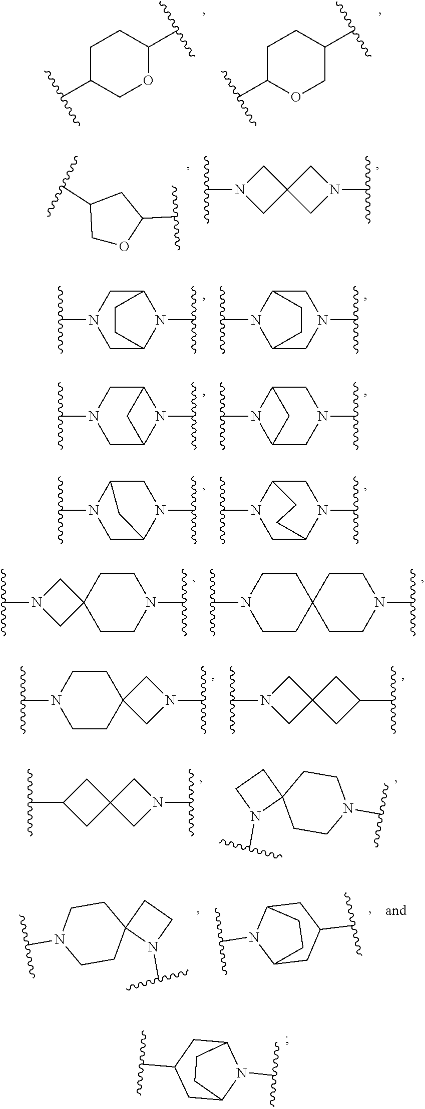

where each ring is substituted as defined therein.
B26. In embodiment B26, the compound for use of any one of embodiments B8 to B25, or a pharmaceutically acceptable salt thereof, is wherein each of the phenylene, heteroarylene, heterocyclylene, bridged heterocyclylene, and spiro heterocyclylene is independently selected from:

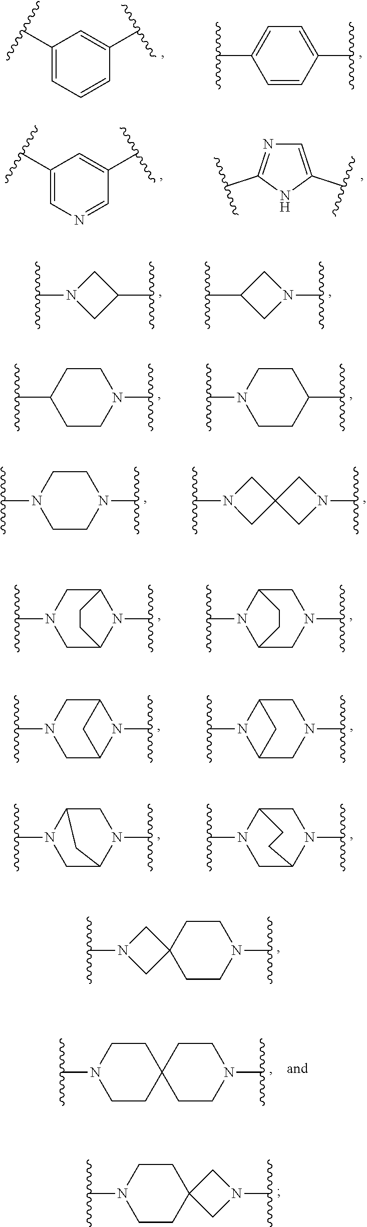

where each ring is substituted as defined therein.
B27. In embodiment B27, the compound for use of any one of embodiments B8 to B26, or a pharmaceutically acceptable salt thereof, is wherein each of phenylene, heteroarylene, heterocyclylene, bridged heterocyclylene, and spiro heterocyclylene is independently selected from:

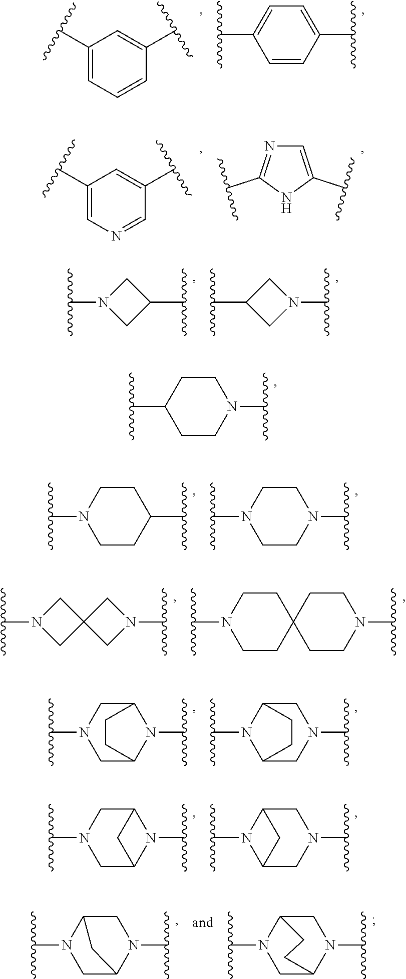

where each ring is substituted as defined therein.
B28. In embodiment B28, the compound for use of any one of embodiments B8 to B27, or a pharmaceutically acceptable salt thereof, is wherein each of the phenylene, heteroarylene, heterocyclylene, bridged heterocyclylene, and spiro heterocyclylene is independently selected from:

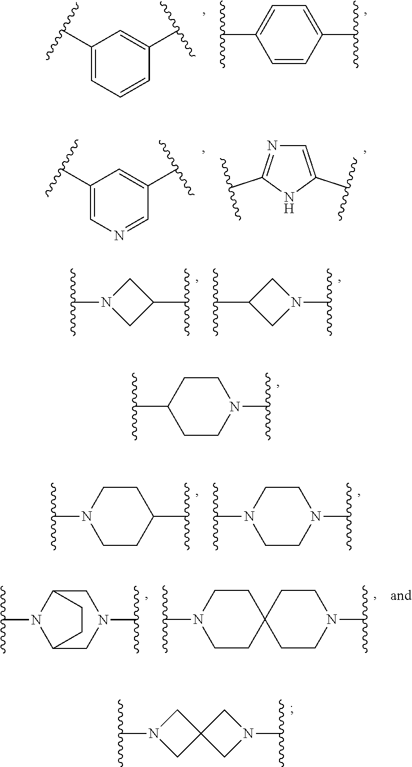

where each ring is substituted as defined therein.
B29. In embodiment B29, the compound for use of any one of embodiments B17 to B28, or a pharmaceutically acceptable salt thereof, is wherein phenylene, heteroarylene, or heterocyclylene is attached to —SO₂— that is attached to Hy of Formula A and are:

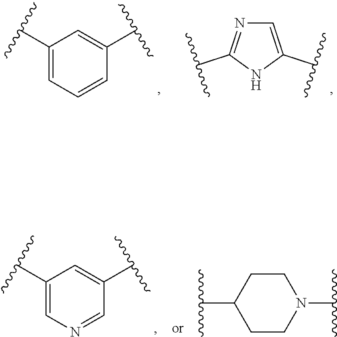 [SMILES: *c1cccc(*)c1 ; *c1cnc(*)[nH]1 ; *c1cncc(*)c1 ; *C1CCN(*)CC1]

respectively, each ring substituted as defined therein. (For sake of clarity the bond on the right-hand side of the ring is attached to —SO₂—). In a subembodiment of embodiment B29,

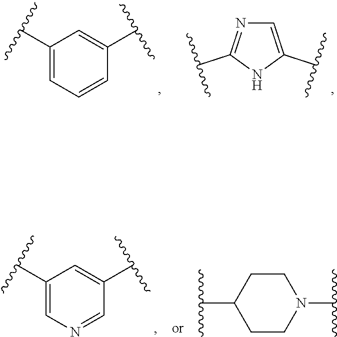 [SMILES: *c1cccc(*)c1 ; *c1cnc(*)[nH]1 ; *c1cncc(*)c1 ; *C1CCN(*)CC1]

is attached to —SO₂—, Hy contains a substitutable nitrogen atom, and the —SO₂— is attached to the substitutable nitrogen of Hy of formula A or A1.
B30. In embodiment B30, the compound for use of any one of embodiments B17 to B29, or a pharmaceutically acceptable salt thereof, is wherein

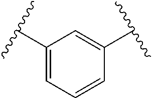 [SMILES: *c1cccc(*)c1]

is attached to —SO₂— that is attached to Hy of Formula A or A1 and is substituted as defined therein. In a subembodiment of embodiment B30,

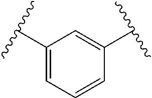 [SMILES: *c1cccc(*)c1]

is attached to —SO₂—, Hy contains a substitutable nitrogen atom, and the —SO₂— is attached to the substitutable nitrogen of Hy of formula A or A1.
B31. In embodiment B31, the compound for use of any one of embodiments B17 to B29, or a pharmaceutically acceptable salt thereof, is wherein

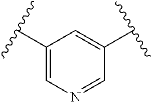 [SMILES: *c1cncc(*)c1]

is attached to —SO₂— that is attached to Hy of Formula A or A1 and is substituted as defined therein. In a subembodiment of embodiment B31,

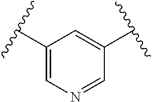 [SMILES: *c1cncc(*)c1]

is attached to —SO₂—, Hy contains a substitutable nitrogen atom, and the —SO₂— is attached to the substitutable nitrogen of Hy of formula A or A1.
B32. In embodiment B32, the compound for use of any one of embodiments B17 to B29, or a pharmaceutically acceptable salt thereof, is wherein

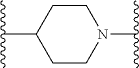 [SMILES: *C1CCN(*)CC1]

is attached to —SO₂— that is attached to Hy of Formula A or A1 and is substituted as defined therein. In a subembodiment of embodiment B29,

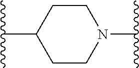 [SMILES: *C1CCN(*)CC1]

is attached to —SO₂—, Hy contains a substitutable nitrogen atom, and the —SO₂— is attached to the substitutable nitrogen of Hy of formula A or A1.
B33. In embodiment B33, the compound for use of any one of embodiments B23b to B29, or a pharmaceutically acceptable salt thereof, is wherein is

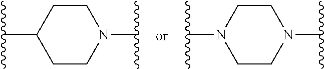 [SMILES: *C1CCN(*)CC1 ; *N1CCN(*)CC1]

is attached to the ring that is attached to SO₂ that is attached to Hy of Formula A or A1. In a subembodiment of embodiment B33,

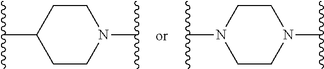 [SMILES: *C1CCN(*)CC1 ; *N1CCN(*)CC1]

is attached to —SO₂—, Hy contains a substitutable nitrogen atom, and the —SO₂— is attached to the substitutable nitrogen of Hy of formula A or A1.
B34. In embodiment B34, the compound for use of any one of embodiments BIA to B3, or a pharmaceutically acceptable salt thereof, is wherein the linker comprises one or more groups independently selected from ether, polyether, thioether, amido, sulfonamido, alkylene, alkenylene, alkynylene, carbonyl, —C(O)O—, —OC(O)—, —NH—, —N(alkyl)-, sulfinyl, ureido, thioureido, bicyclic heterocyclylene, and fused heterocyclylene; wherein bicyclic heterocyclylene and fused heterocyclylene are optionally substituted with one, two, or three substituents independently selected from deuterium, alkyl, alkoxy, halo, haloalkyl, haloalkoxy, hydroxy, cyano, amino, alkylamino, and dialkylamino and each alkylene is optionally substituted with one or two substituents wherein one of the substituents is deuterium and the other of the two substituents is deuterium, haloalkyl, hydroxy, alkoxy, cyano, cycloalkyl, heterocyclyl, aryl, or monocyclic heteroaryl, wherein cycloalkyl, heterocyclyl, aryl, and monocyclic heteroaryl are optionally substituted with one or two substituents independently selected from alkyl, alkoxy, halo, haloalkyl, haloalkoxy, and cyano. In one embodiment, the linker comprises five or six groups which are independently selected.
B35. In embodiment B35, the compound for use of any one of embodiments B1A to B3 and B34, or a pharmaceutically acceptable salt thereof, is wherein the linker comprises one to four groups independently selected from those stated in embodiment B34. In one embodiment, the linker comprises four groups which are independently selected.
B36. In embodiment B36, the compound for use of any one of embodiments BIA to B3 and B34, or a pharmaceutically acceptable salt thereof, is wherein the linker comprises one to three groups independently selected from those stated in embodiment B34. In one embodiment, the linker comprises three groups which are independently selected.
B37. In embodiment B37, the compound for use of any one of embodiments B1A to B3 and B34, or a pharmaceutically acceptable salt thereof, is wherein the linker comprises one or two groups independently selected from those stated in embodiment B34. In one embodiment, the linker comprises two groups which are independently selected. In one embodiment, the linker comprises one group.
B38. In embodiment B38, the compound for use of any one of embodiments B4 to B33, or a pharmaceutically acceptable salt thereof, is wherein the linker further comprises one or more groups independently selected from ether, polyether, thioether, —NH—, —N(alkyl)-, amido, sulfonamido, alkylene, alkenylene, alkynylene, carbonyl, —C(O)O—, —OC(O)—, sulfinyl, ureido, thioureido, bicyclic heterocyclylene, and fused heterocyclylene; wherein bicyclic heterocyclylene and fused heterocyclylene are optionally substituted with one, two, or three substituents independently selected from deuterium, alkyl, alkoxy, halo, haloalkyl, haloalkoxy, hydroxy, cyano, amino, alkylamino, and dialkylamino and each alkylene is optionally substituted with one or two substituents wherein one of the substituents is deuterium and the other of the two substituents is deuterium, haloalkyl, hydroxy, alkoxy, cyano, cycloalkyl, heterocyclyl, aryl, or monocyclic heteroaryl, wherein cycloalkyl, heterocyclyl, aryl, and monocyclic heteroaryl are optionally substituted with one or two substituents independently selected from alkyl, alkoxy, halo, haloalkyl, haloalkoxy, and cyano.
B39. In embodiment B39, the compound for use of any one of embodiments B38, or a pharmaceutically acceptable salt thereof, is wherein the linker comprises one to four groups independently selected from those stated in embodiment B38. In one embodiment, the linker comprises four groups which are independently selected.
B40. In embodiment B40, the compound for use of any one of embodiments B38 and B39, or a pharmaceutically acceptable salt thereof, is wherein the linker comprises one to three groups independently selected from those stated in embodiment B38. In one embodiment, the linker comprises three groups which are independently selected.
B41. In embodiment B41, the compound for use of any one of embodiments B38 to B40, or a pharmaceutically acceptable salt thereof, is wherein the linker comprises one or two groups independently selected from those stated in embodiment B38. In one embodiment, the linker comprises two groups which are independently selected.
B42. In embodiment B42, the compound for use of any one of embodiments B38 to B41, or a pharmaceutically acceptable salt thereof, is wherein the linker comprises one group selected from those stated in embodiment B38.
B43. In embodiment B43, the compound for use of any one of embodiments B34 to B42, or a pharmaceutically acceptable salt thereof, is wherein the each alkylene is unsubstituted.
B44. In embodiment B44, the compound for use of any one of embodiments B34 to B42, or a pharmaceutically acceptable salt thereof, is wherein the one or two, preferably one alkylene is substituted as defined therein.
B45. In embodiment B45, the compound for use of any one of embodiments B34 to B44, or a pharmaceutically acceptable salt thereof, is wherein the group(s) stated therein are independently selected from ether, polyether, —NH—, —N(alkyl)-, amido, sulfonamido, alkylene, alkenylene, alkynylene, carbonyl, —C(O)O—, —OC(O)—, ureido, thioureido, bicyclic heterocyclylene, and fused heterocyclylene wherein bicyclic heterocyclylene, and fused heterocyclylene.
B46. In embodiment B46, the compound for use of any one of embodiments B34 to B45, or a pharmaceutically acceptable salt thereof, is wherein the group(s) stated therein are independently selected from ether, polyether, —NH—, —N(alkyl)-, amido, sulfonamido, alkylene, alkenylene, alkynylene, carbonyl, —C(O)O, —OC(O), bicyclic heterocyclylene, and fused heterocyclylene wherein bicyclic heterocyclylene, and fused heterocyclylene.
B47. In embodiment B47, the compound for use of any one of embodiments B34 to B46, or a pharmaceutically acceptable salt thereof, is wherein the group(s) stated therein are independently selected from ether, polyether, —NH—, —N(alkyl)-, amido, sulfonamido, alkylene, alkenylene, alkynylene, carbonyl, —C(O)O, —OC(O), and each alkylene is substituted or unsubstituted as stated therein.
B48. In embodiment B48, the compound for use of any one of embodiments B34 to B47, or a pharmaceutically acceptable salt thereof, is wherein the one or more group(s) stated therein are independently selected from ether, polyether, —NH—, —N(methyl)-, —NHC(O)—, —C(O)NH—, —N(methyl)C(O), —C(O)N(methyl)-, —NHSO₂—, —SO₂NH—, —N(methyl) SO₂, —SO₂N(methyl)-, —NHC(O)NH—, —NHSO₂NH—, methylene, ethylene, propylene, butylene, pentylene, ethenylene, propenylene, acetylene, propynylene, carbonyl, —C(O)O—, and —OC(O)—.
B49. In embodiment B49, the compound for use of any one of embodiments B34 to B48, or a pharmaceutically acceptable salt thereof, is wherein the one or more group(s) stated therein are independently selected from ether, —NH—, —N(methyl)-, methylene, ethylene, propylene, butylene, pentylene, ethenylene, propenylene, acetylene, propynylene, and carbonyl.
B50. In embodiment B50, the compound for use of any one of embodiments B34 to B49, or a pharmaceutically acceptable salt thereof, is wherein the one or more group(s) stated therein are independently selected from ether, —NH—, —N(methyl)-, methylene, ethylene, propylene, butylene, pentylene, ethenylene, propenylene, acetylene, and propynylene.
B51. In embodiment B51, the compound for use of any one of embodiments BIA to B3, or a pharmaceutically acceptable salt thereof, is wherein the linker comprises or consists of one or more, preferably comprises or consists of one to seven groups, independently selected from those disclosed in Table A:

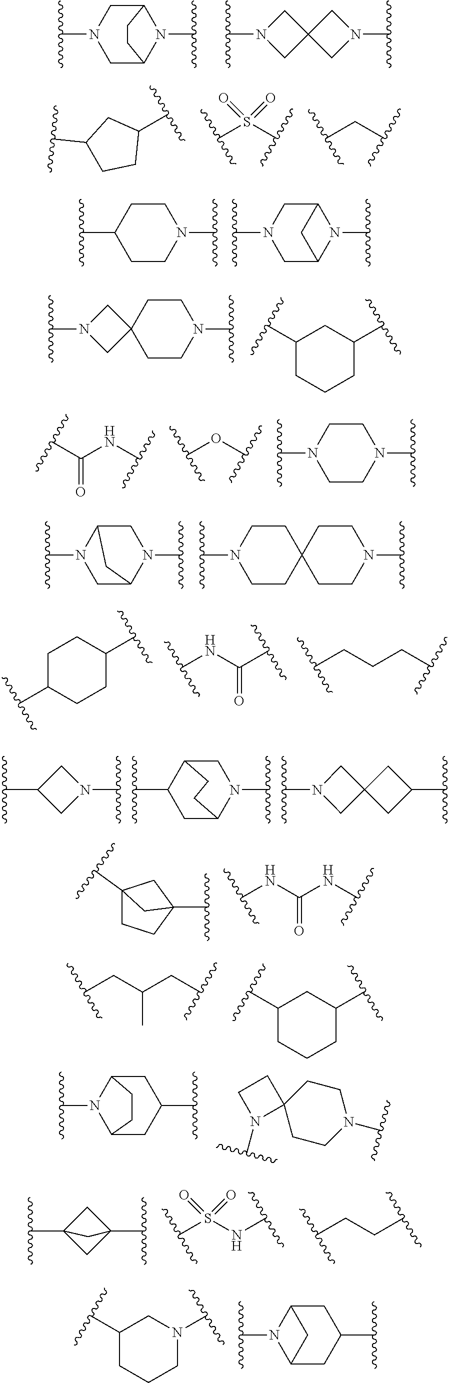

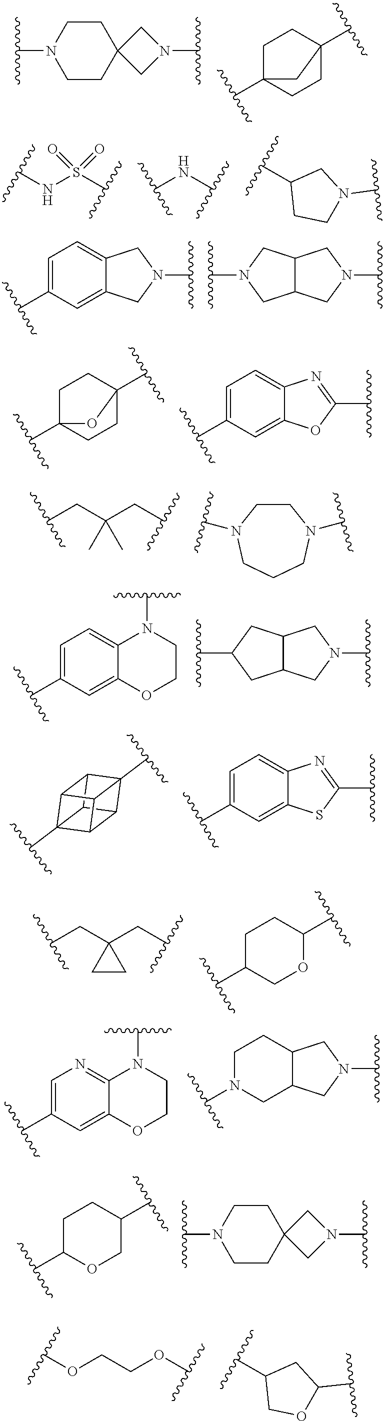

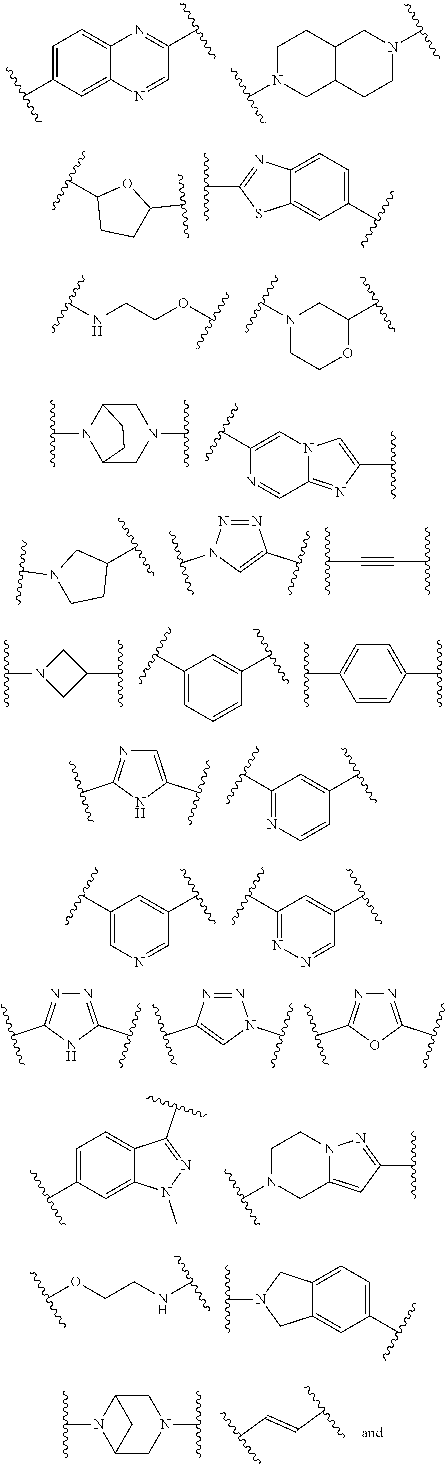

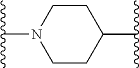 [SMILES: *C1CCN(*)CC1]

and isomers thereof;
wherein each ring is optionally be substituted with one, two, or three substituents independently selected from deuterium, alkyl, alkoxy, halo, haloalkyl, haloalkoxy, hydroxy, cyano, carboxy, alkoxycarbonyl, amino, alkylamino, and dialkylamino and each alkylene is optionally substituted with one or two substituents wherein one of the substituent is deuterium and the other of the two substituent is deuterium, haloalkyl, hydroxy, alkoxy, cyano, cycloalkyl, heterocyclyl, aryl, or monocyclic heteroaryl, wherein cycloalkyl, heterocyclyl, aryl, and monocyclic heteroaryl are substituted with one or two substituents independently selected from hydrogen, alkyl, alkoxy, halo, haloalkyl, haloalkoxy, and cyano;
wherein the isomers of the groups in Table A, where applicable, can be cis and/or trans isomers, and/or R and/or S isomers, and/or other geometric isomers;
wherein the left side in the groups in Table A is ultimately attached to the E3 ubiquitin ligase ligand and the right side in the groups in Table A is ultimately attached to Hy; or
   alternatively, the left side in the groups in Table A is ultimately attached to Hy and the right side in the groups in Table A is ultimately attached to the E3 ubiquitin ligase ligand.
B51a. In embodiment B51a, the compound for use of any one of embodiments B1A to B3, or a pharmaceutically acceptable salt thereof, is wherein the linker comprises or consists of one or more, preferably comprises or consists of one to seven groups, independently selected from those disclosed in Table A:

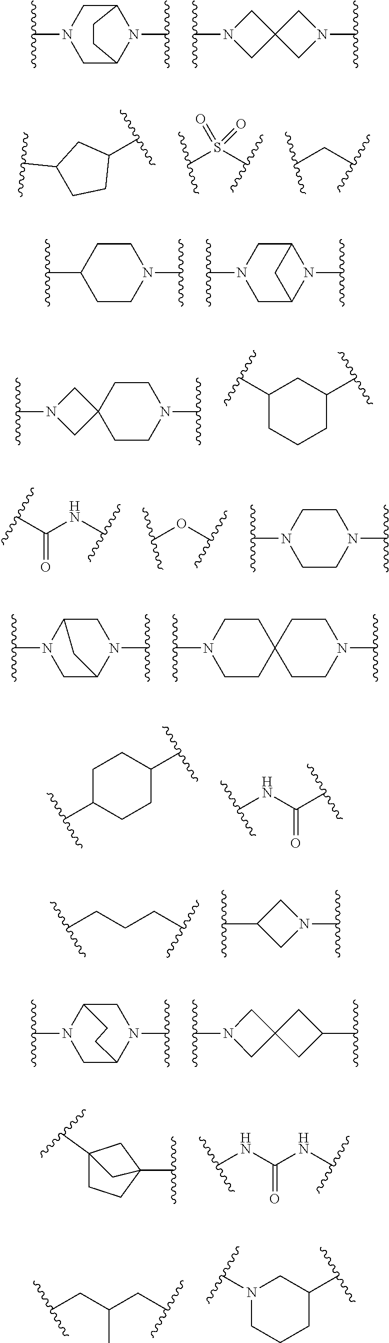

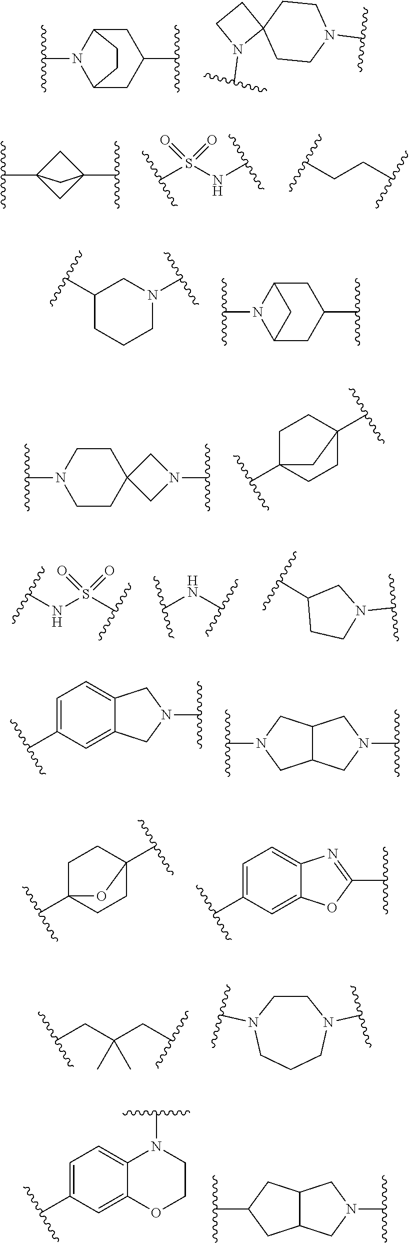

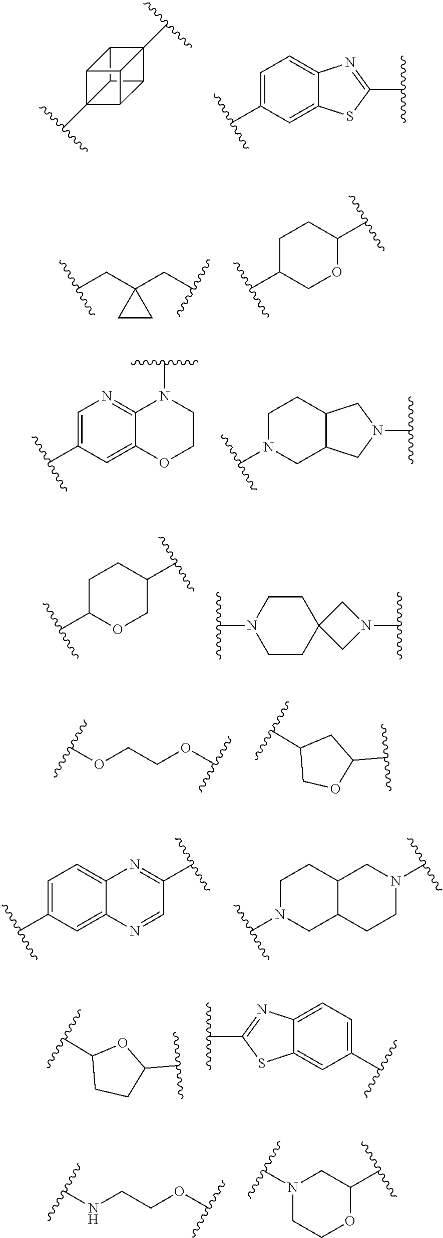

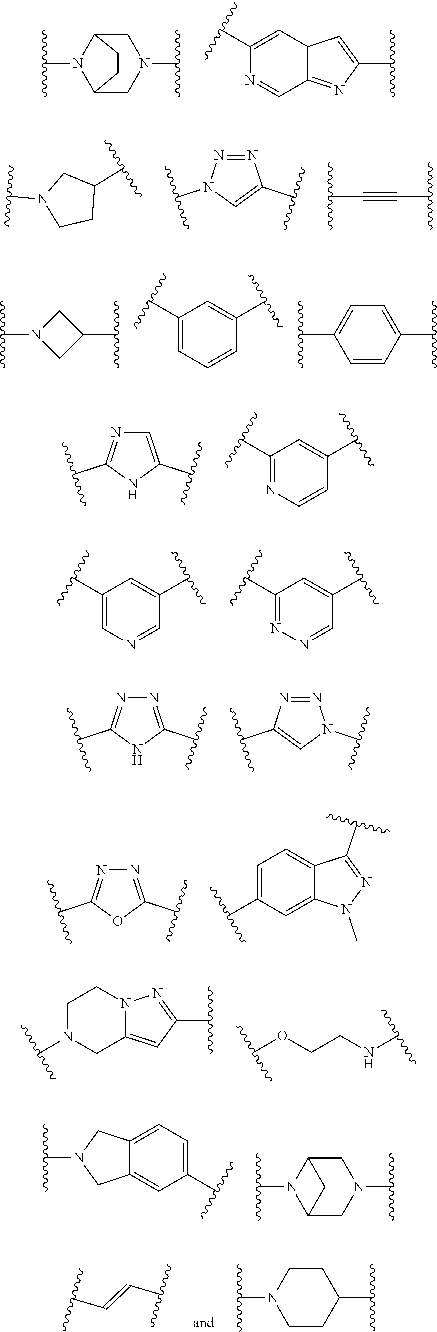

and isomers thereof;
wherein each ring is optionally be substituted with one, two, or three substituents independently selected from deuterium, alkyl, alkoxy, halo, haloalkyl, haloalkoxy, hydroxy, cyano, amino, alkylamino, and dialkylamino and each alkylene is optionally substituted with one or two substituents wherein one of the substituent is deuterium and the other of the two substituent is deuterium, haloalkyl, hydroxy, alkoxy, cyano, cycloalkyl, heterocyclyl, aryl, or monocyclic heteroaryl, wherein cycloalkyl, heterocyclyl, aryl, and monocyclic heteroaryl are substituted with one or two substituents independently selected from hydrogen, alkyl, alkoxy, halo, haloalkyl, haloalkoxy, and cyano;
wherein the isomers of the groups in Table A, where applicable, can be cis and/or trans isomers, and/or R and/or S isomers, and/or other geometric isomers;
wherein the left side in the groups in Table A is ultimately attached to the E3 ubiquitin ligase ligand and the right side in the groups in Table A is ultimately attached to Hy; or
alternatively, the left side in the groups in Table A is ultimately attached to Hy and the right side in the groups in Table A is ultimately attached to the E3 ubiquitin ligase ligand.
B52. In embodiment B52, the compound for use of embodiment B51, or a pharmaceutically acceptable salt thereof, is wherein the linker comprises or consists of one group selected from those disclosed in Table A.
B52-1. In embodiment B52-1, the compound for use of embodiment B51a, or a pharmaceutically acceptable salt thereof, is wherein the linker comprises or consists of one group selected from those disclosed in Table A.
B52a. In embodiment B52a, the compound for use of embodiment B51, or a pharmaceutically acceptable salt thereof, is wherein the linker comprises or consists of two groups independently selected from those disclosed in Table A.
B52a-1. In embodiment B52a-1, the compound for use of embodiment B51a, or a pharmaceutically acceptable salt thereof, is wherein the linker comprises or consists of two groups independently selected from those disclosed in Table A.
B53. In embodiment B53, the compound for use of embodiment B51, or a pharmaceutically acceptable salt thereof, is wherein the linker comprises or consists of three groups independently selected from those disclosed in Table A.
B53a. In embodiment B53a, the compound for use of embodiment B51a, or a pharmaceutically acceptable salt thereof, is wherein the linker comprises or consists of three groups independently selected from those disclosed in Table A.
B54. In embodiment B54, the compound for use of embodiment B51, or a pharmaceutically acceptable salt thereof, is wherein the linker comprises or consists of four groups independently selected from those disclosed in Table A.
B54a. In embodiment B54a, the compound for use of embodiment B51a, or a pharmaceutically acceptable salt thereof, is wherein the linker comprises or consists of four groups independently selected from those disclosed in Table A.
B55. In embodiment B55, the compound for use of embodiment B51, or a pharmaceutically acceptable salt thereof, is wherein the linker comprises or consists of five groups independently selected from those disclosed in Table A.
B55a. In embodiment B55a, the compound for use of embodiment B51a, or a pharmaceutically acceptable salt thereof, is wherein the linker comprises or consists of five groups independently selected from those disclosed in Table A.
B56. In embodiment B56, the compound for use of any one of embodiments B51 to B55a, or a pharmaceutically acceptable salt thereof, is wherein at least one of the groups is —SO₂—.
B57. In embodiment B57, the compound for use of any one of embodiments B51 to B56, or a pharmaceutically acceptable salt thereof, is wherein one of the groups is —SO₂—.
B58. In embodiment B58, the compound for use of embodiment B56 or B57, or a pharmaceutically acceptable salt thereof, is wherein the —SO₂— is attached to Hy of any one of embodiments BIA to B3. In a subembodiment, Hy contains a substitutable nitrogen and the —SO₂— is attached to the substitutable nitrogen of Hy of any one of embodiments B1A to B3.
B59. In embodiment B59, the compound for use of any one of embodiments B51 to B58, or a pharmaceutically acceptable salt thereof, is wherein one, two, or three of the groups, preferably two or three of the groups, are rings independently selected from those disclosed in Table A.
B60. In embodiment B60, the compound for use of embodiment B59, or a pharmaceutically acceptable salt thereof, is wherein one of the rings (designated as ring (ia)) is attached to —SO₂— that is attached to Hy, preferably ring (ia) is:

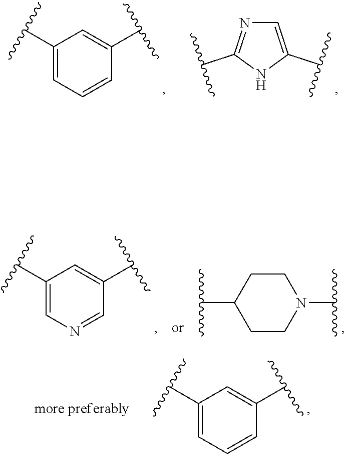 [SMILES: *c1cccc(*)c1 ; *c1cnc(*)[nH]1 ; *c1cncc(*)c1 ; *C1CCN(*)CC1 ; *c1cccc(*)c1]

where ring (ia) is optionally substituted as defined, preferably, ring (ia) is:

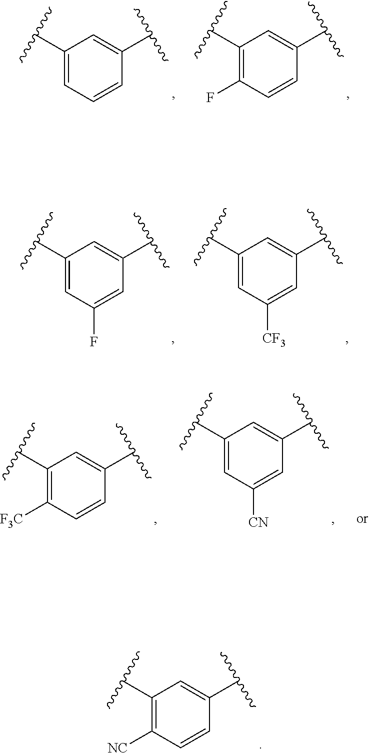

In a subembodiment, ring (ia) is attached to —SO₂—, Hy contains a substitutable nitrogen atom, and the —SO₂— is attached to the substitutable nitrogen of Hy of formula A or A1, preferably ring (ia) is:

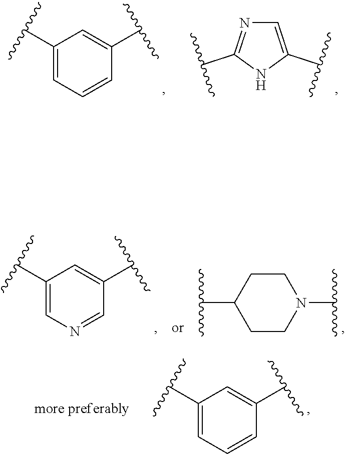 [SMILES: *c1cccc(*)c1 ; *c1cnc(*)[nH]1 ; *c1cncc(*)c1 ; *C1CCN(*)CC1 ; *c1cccc(*)c1]

where ring (ia) is optionally substituted as defined, preferably, ring (ia) is:

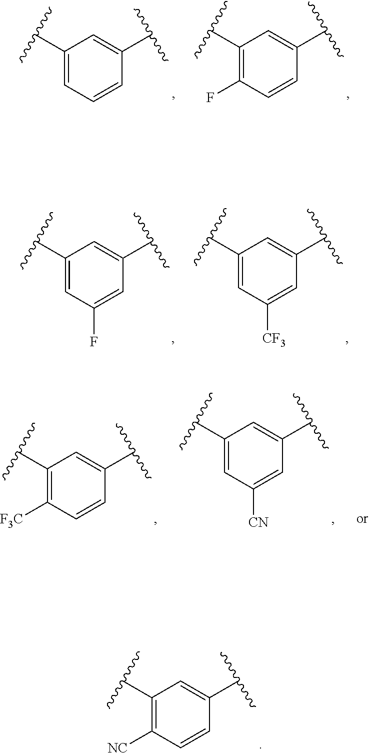

B61. In embodiment B61, the compound for use of embodiment B60, and embodiments therein, or a pharmaceutically acceptable salt thereof, is wherein a second ring (referred to herein as ring (ib)) (when the linker has two or more rings) is attached to the ring that is attached to the —SO₂— attached to Hy. In a subembodiment, ring (ib) is attached to the ring that is attached to the —SO₂—, Hy contains a substitutable nitrogen atom, and the —SO₂— is attached to the substitutable nitrogen of Hy of formula A or A1.
B62. In embodiment B62, the compound for use embodiment B61, or a pharmaceutically acceptable salt thereof, is wherein the second ring (ib) is selected from the group consisting of

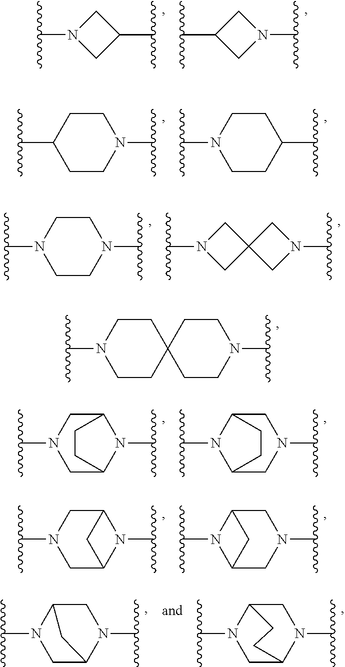

where each ring (ib) is substituted as stated therein.
B63. In embodiment B63, the compound for use embodiment B51 to B62, or a pharmaceutically acceptable salt thereof, is wherein one of the groups is:

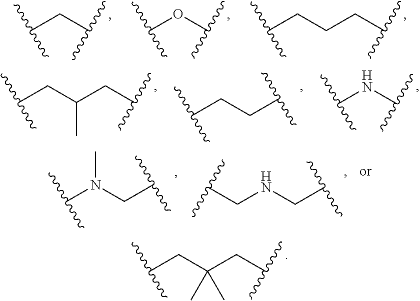

B64. In embodiment B64, the compound for use of any one of embodiments B1A to B3, or a pharmaceutically acceptable salt thereof, is wherein the linker comprises a group selected from:

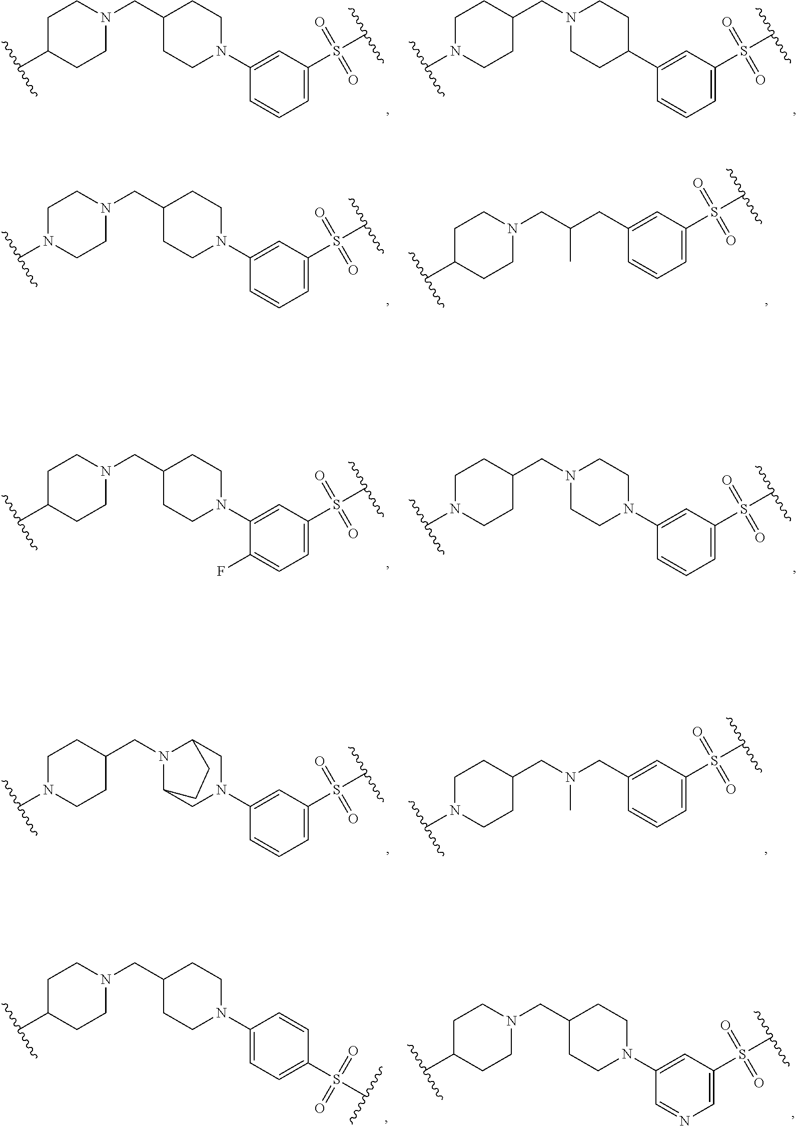

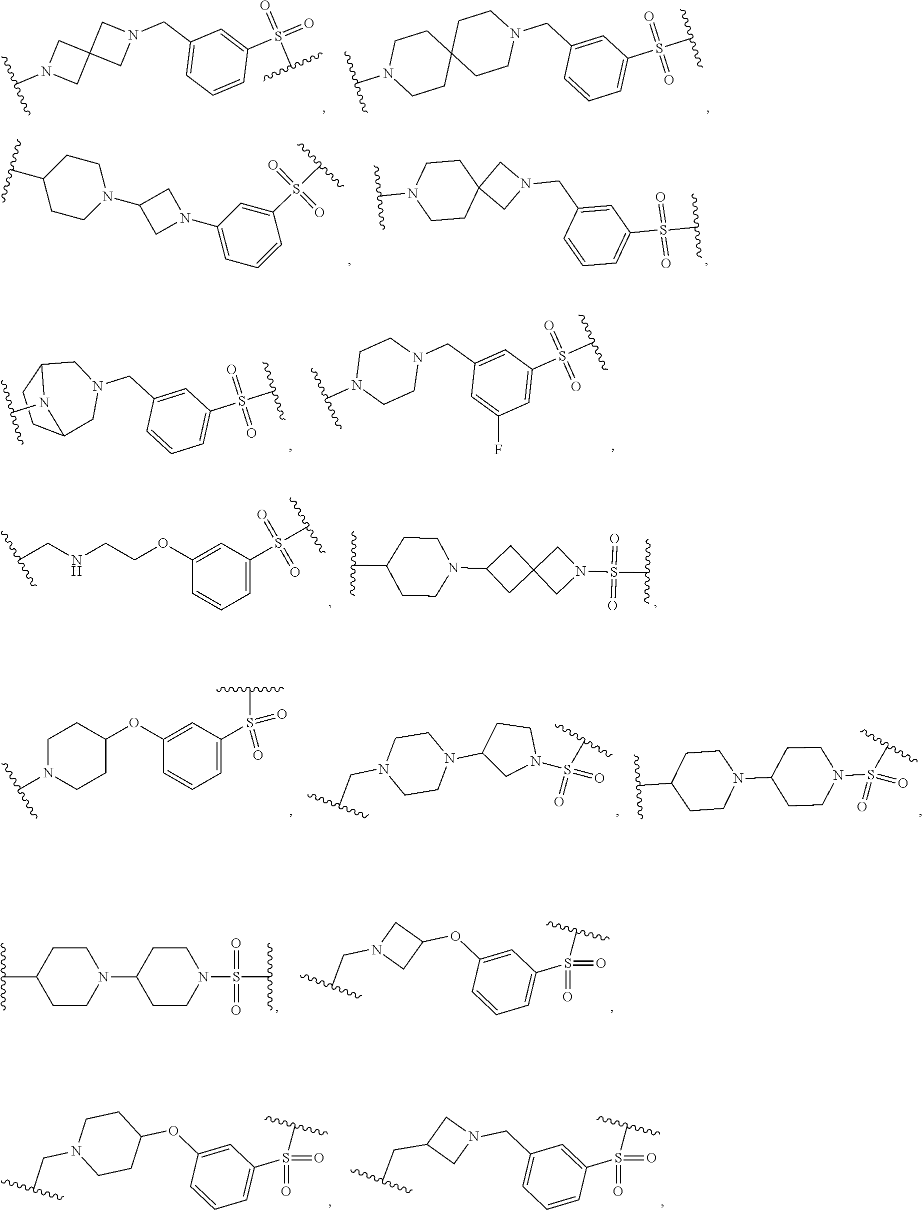

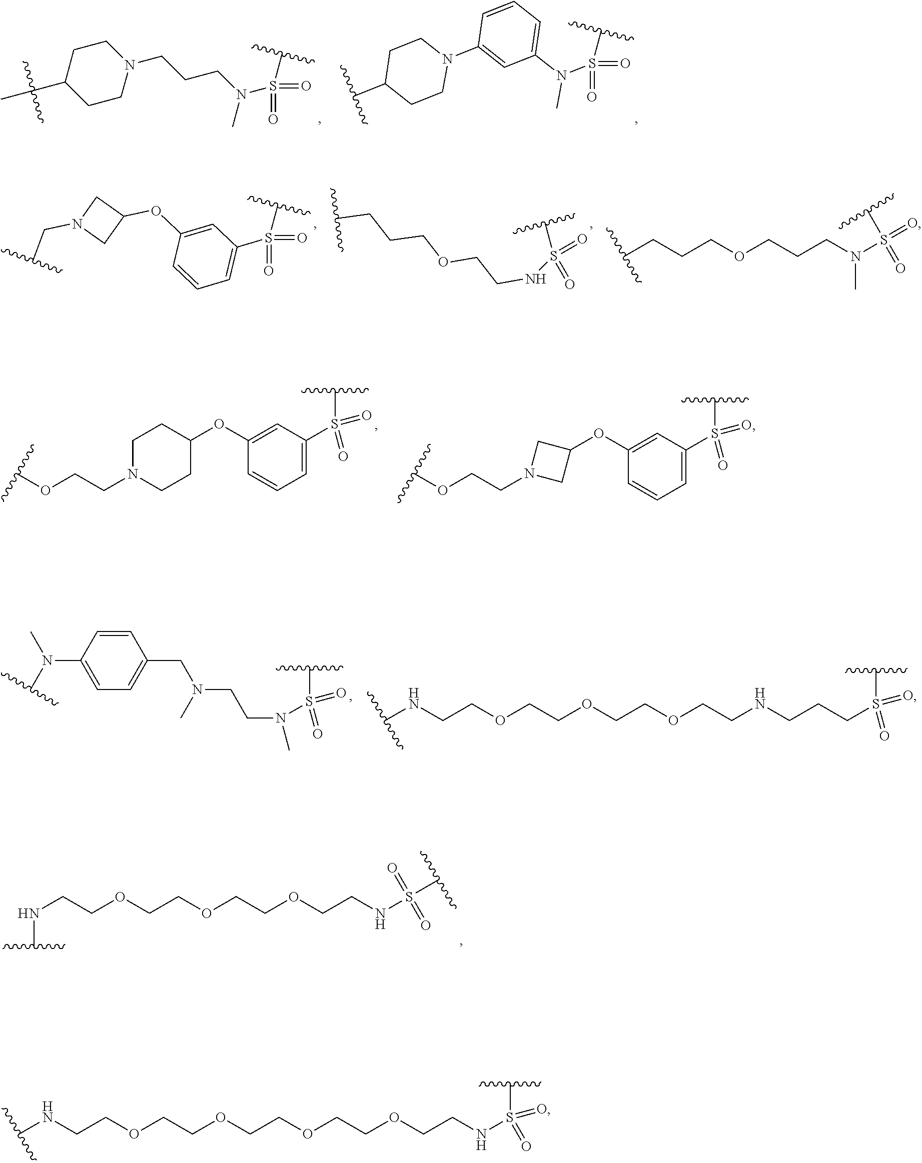

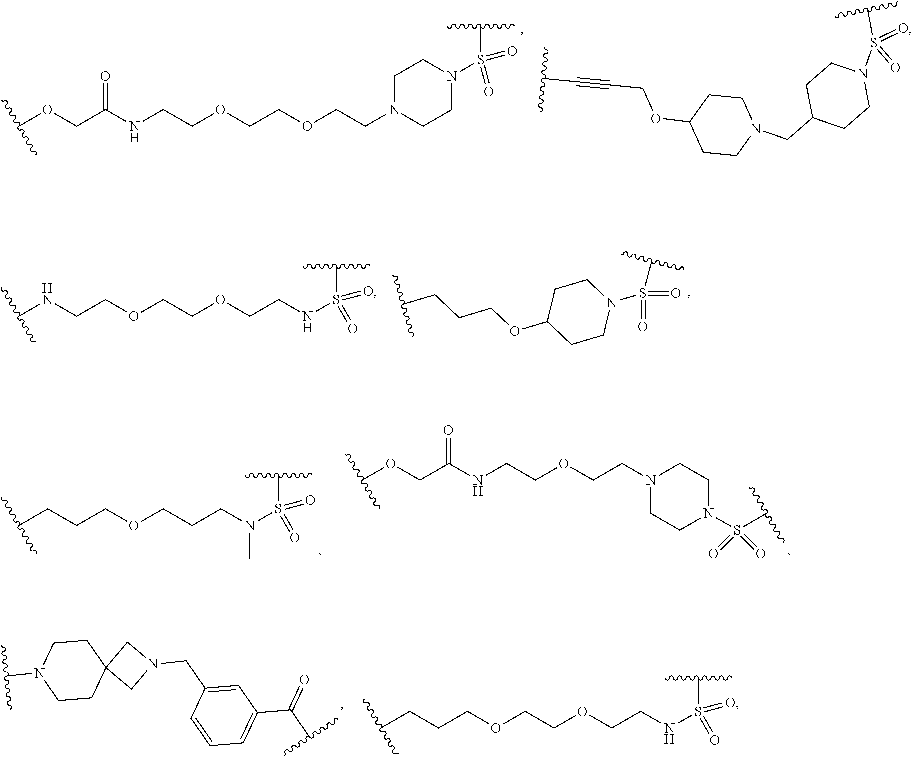

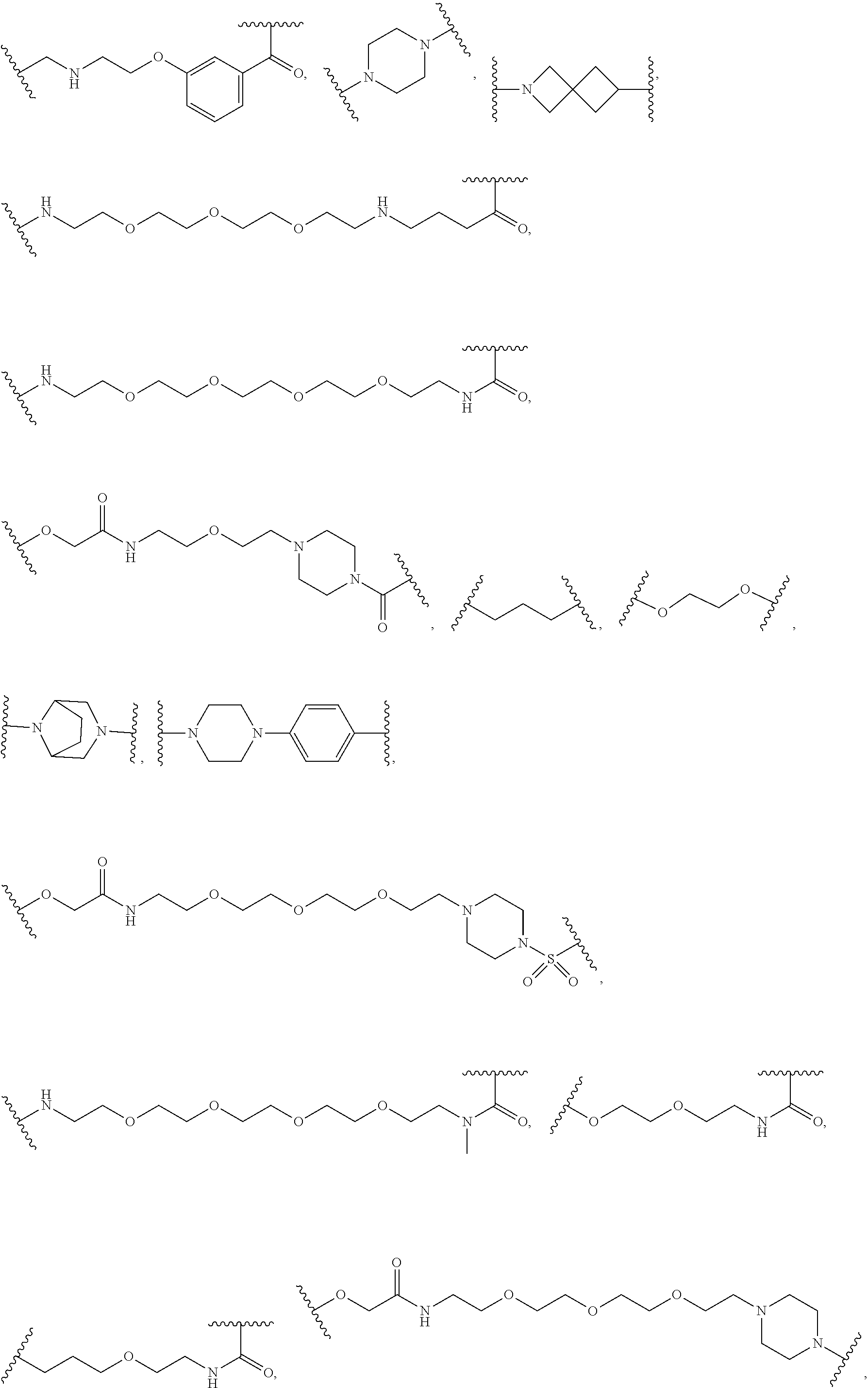

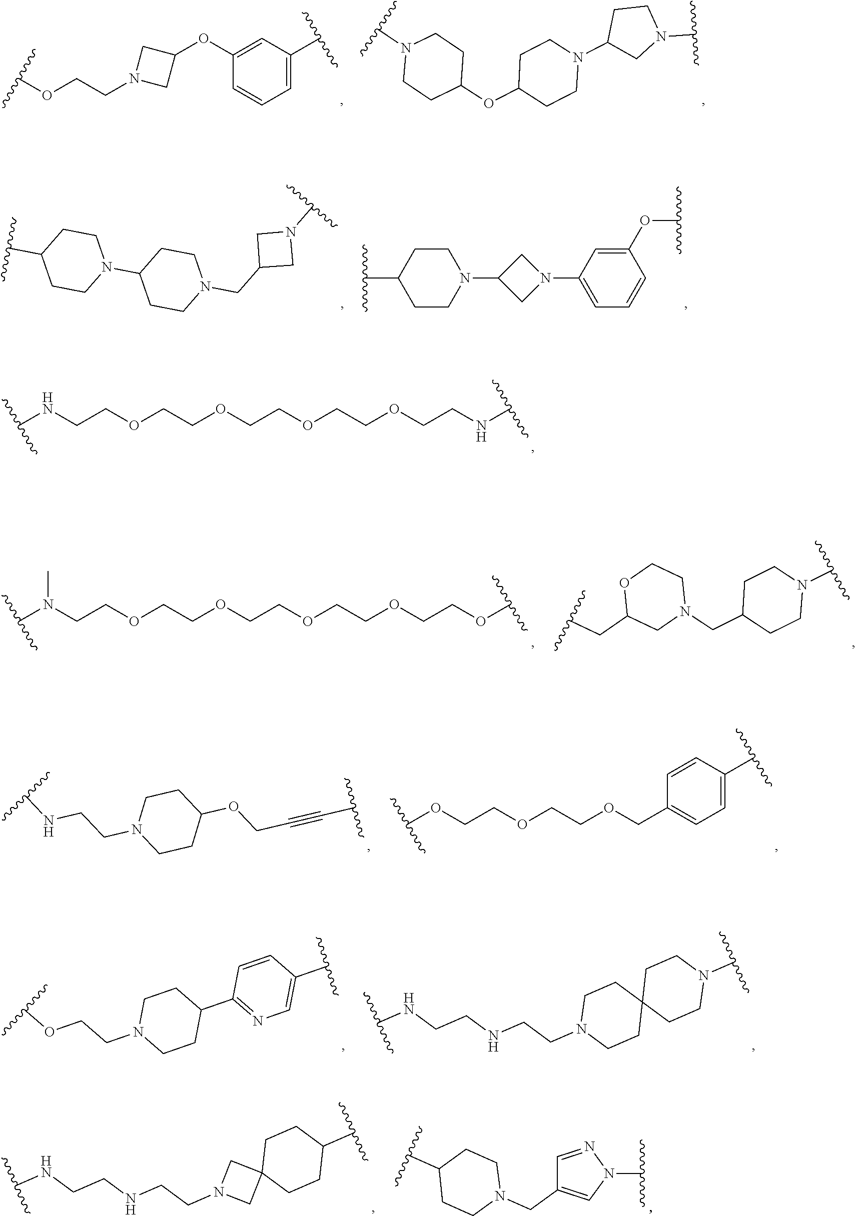

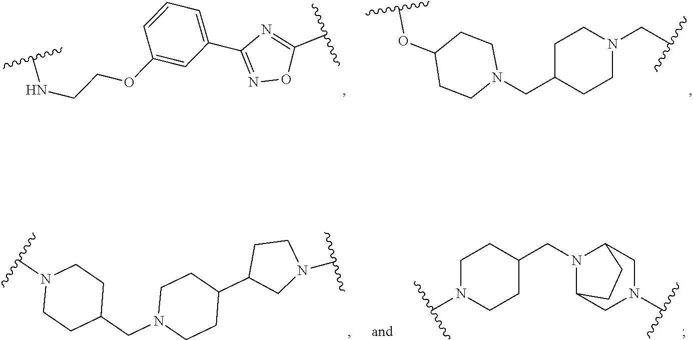 [SMILES: *NCCOc1cccc(-c2noc(*)n2)c1 ; *CN1CCC(CN2CCC(O*)CC2)CC1 ; *N1CCC(CN2CCC(C3CCN(*)C3)CC2)CC1 ; *N1CCC(CN2C3CCC2CN(*)C3)CC1]

where, in the structures above, the right side of the linker is attached to Hy. In a subembodiment, Hy contains a substitutable nitrogen and the right side of the linker is attached to the substitutable nitrogen of Hy.
B65. In embodiment B65, the compound for use of any one of embodiments B1A to B3, or a pharmaceutically acceptable salt thereof, is wherein the linker is selected from:

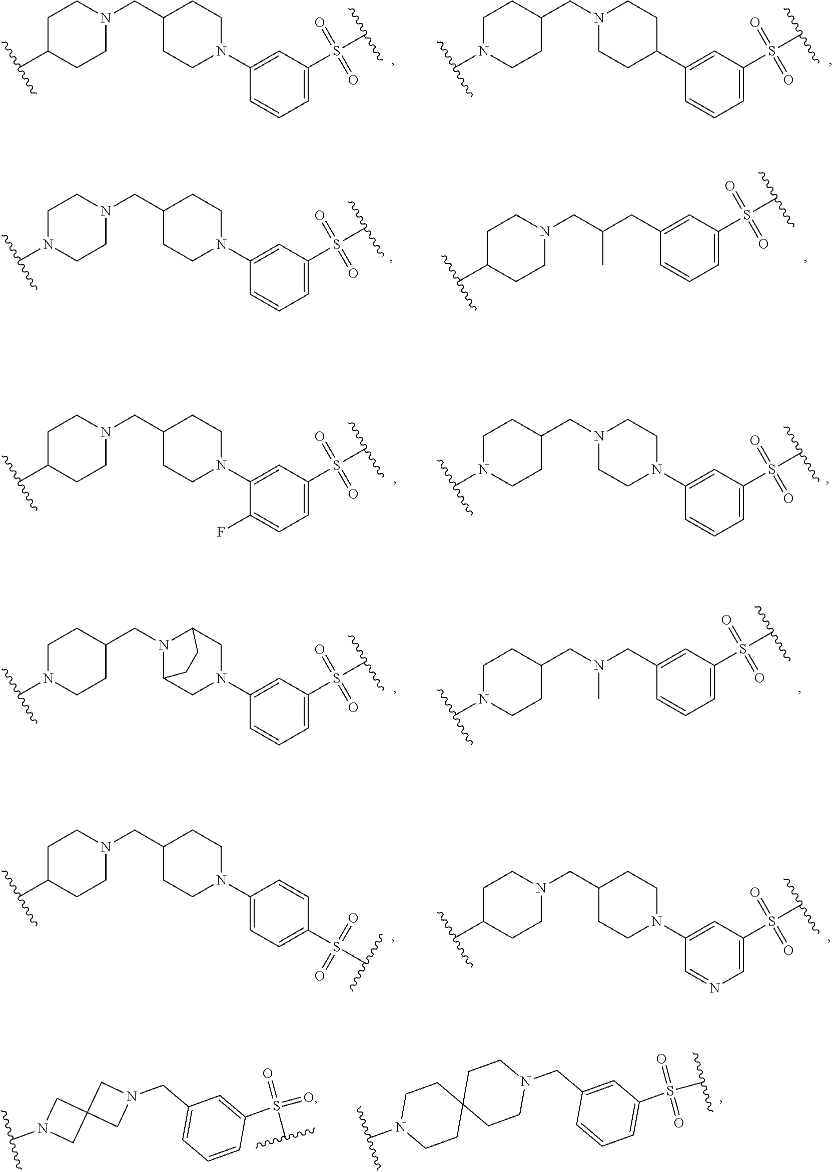

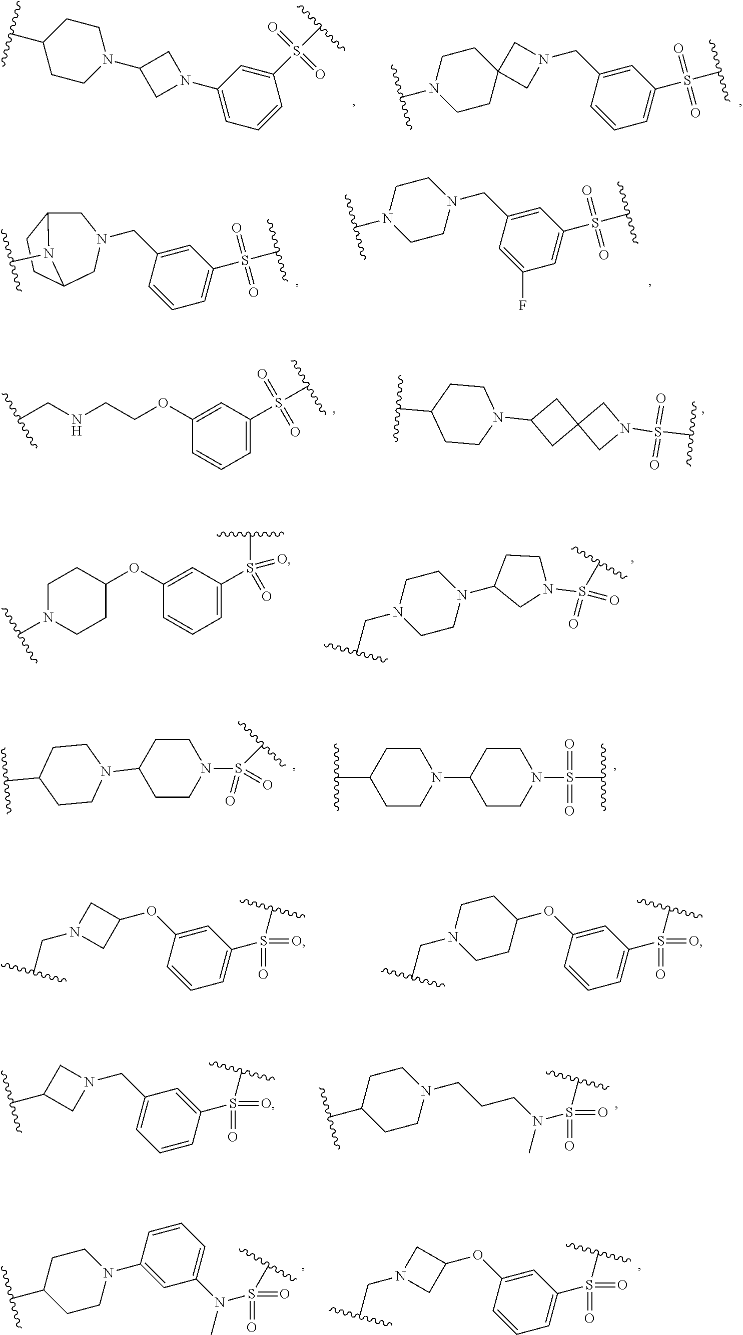

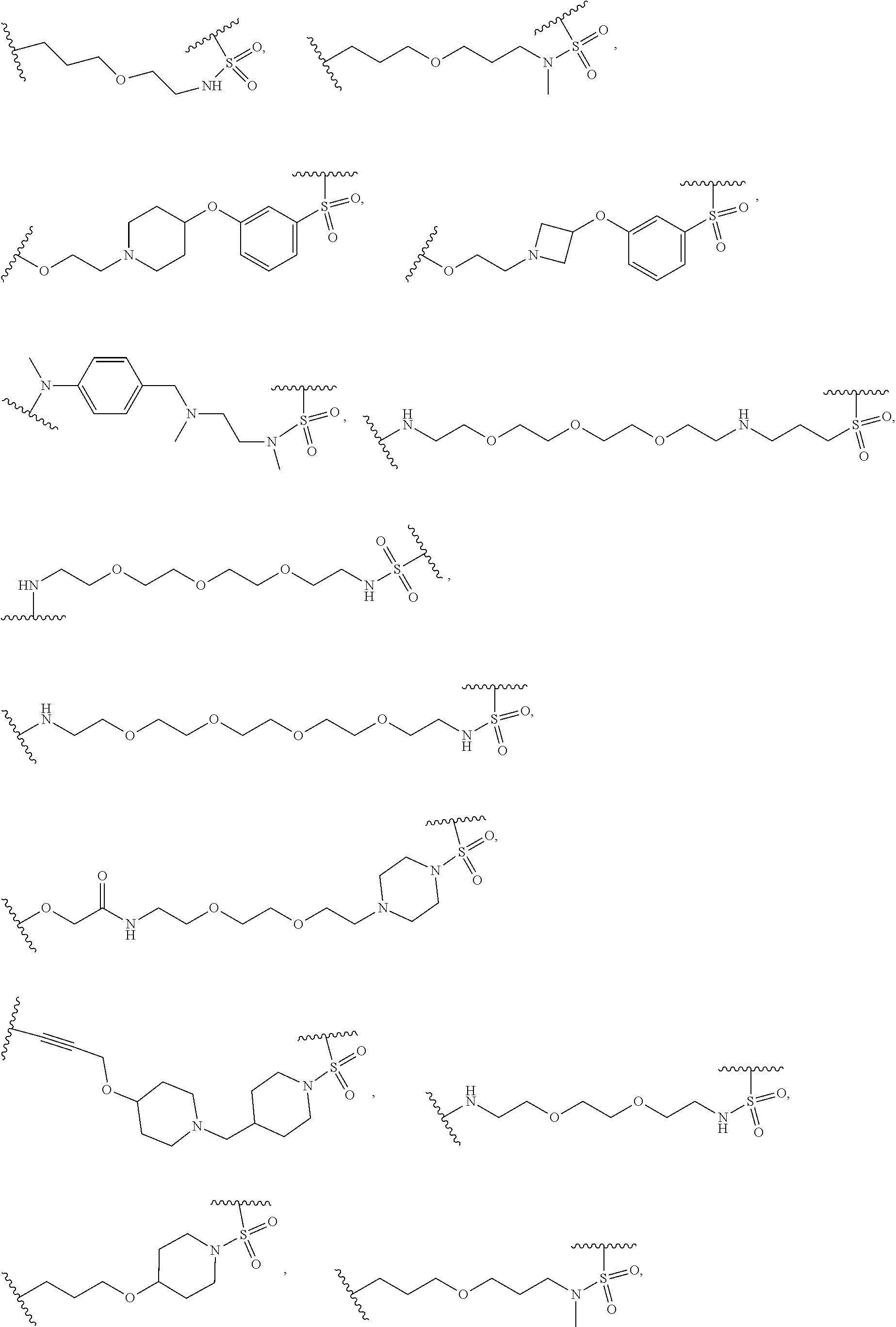

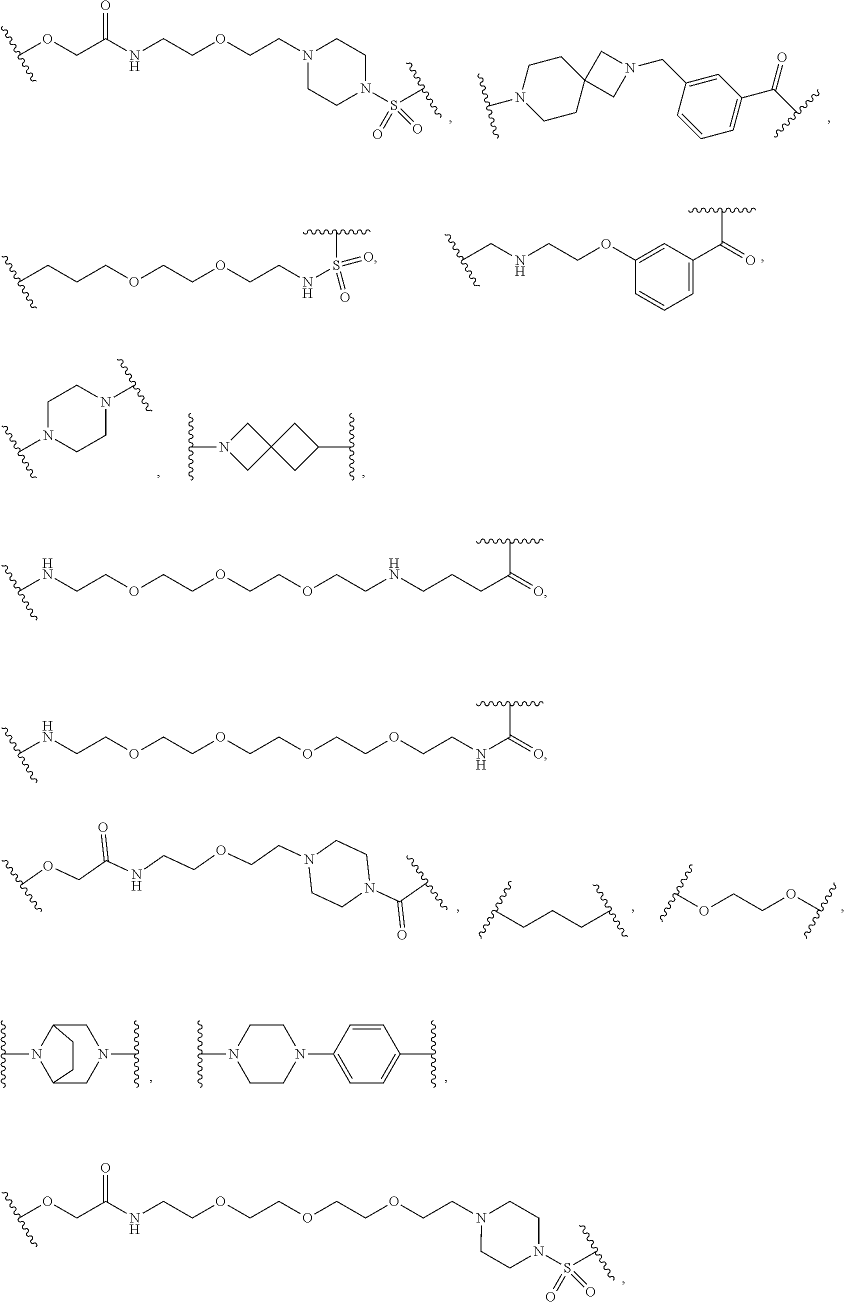

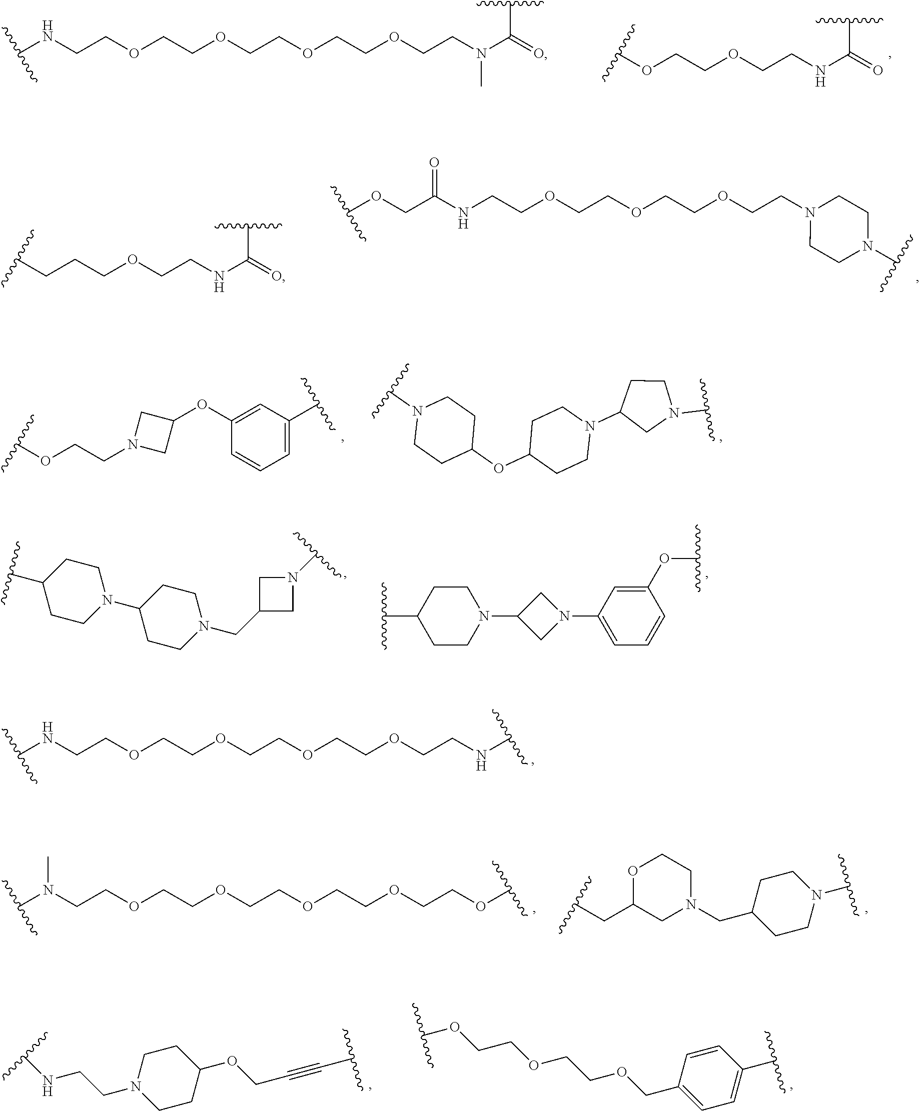

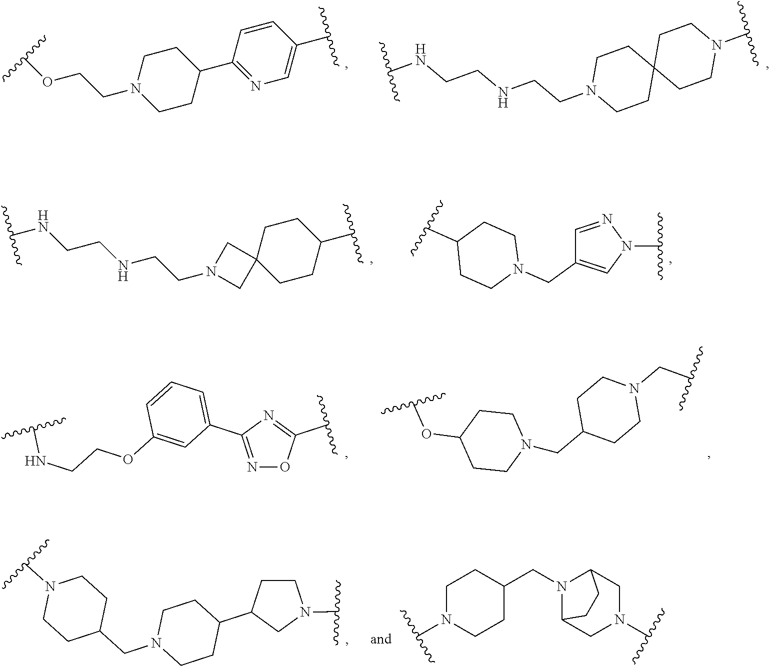

where, in the structures above, the right side of the linker is attached to Hy and left side is attached to E3 ubiquitin ligase ligand. In a subembodiment, Hy contains a substitutable nitrogen and the right side of the linker is attached to the substitutable nitrogen of Hy.
B66. In embodiment B66, the compound for use of any one of embodiments B1A to B3 and B65, or a pharmaceutically acceptable salt thereof, is wherein the linker is selected from:

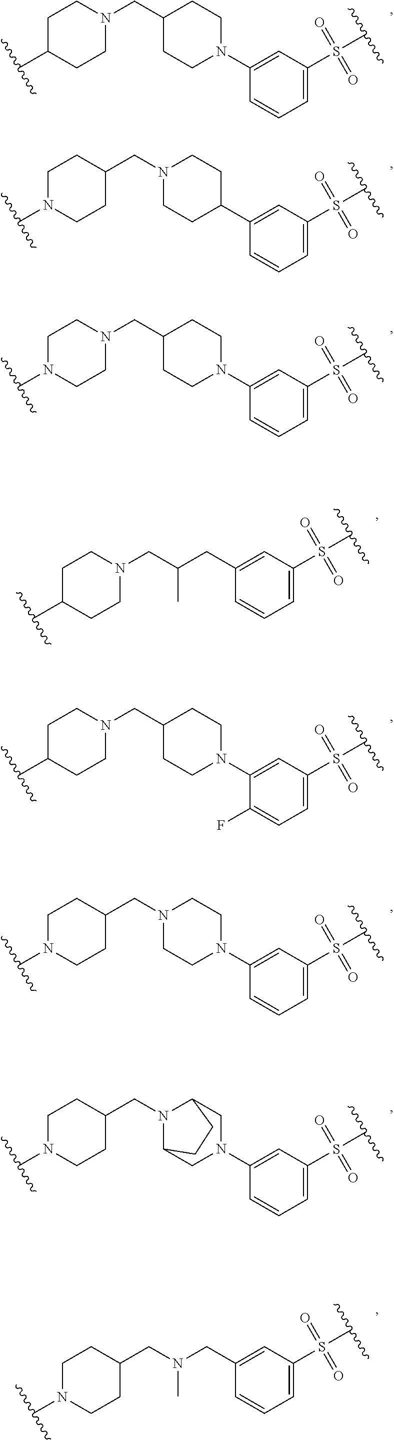

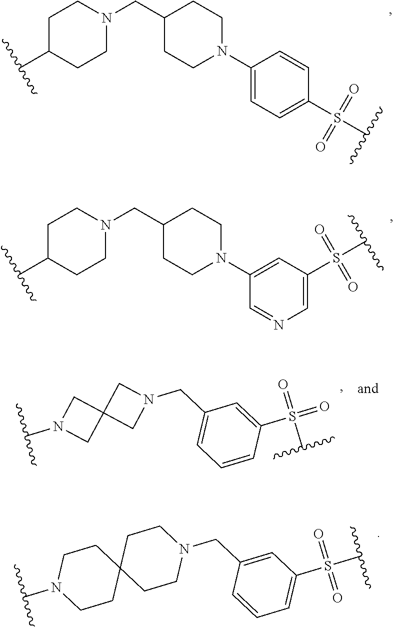 [SMILES: *C1CCN(CC2CCN(c3ccc(S(*)(=O)=O)cc3)CC2)CC1 ; *C1CCN(CC2CCN(c3cncc(S(*)(=O)=O)c3)CC2)CC1 ; *N1CC2(C1)CN(Cc1cccc(S(*)(=O)=O)c1)C2 ; *N1CCC2(CC1)CCN(Cc1cccc(S(*)(=O)=O)c1)CC2]

In a first subembodiment, in the structures above, the right side of the linker is attached to Hy. In a second subembodiment, Hy contains a substitutable nitrogen and the right side of the linker is attached to the substitutable nitrogen of Hy.
B67. In embodiment B67, the compound for use of any one of embodiments B3 to B66, or a pharmaceutically acceptable salt thereof, is wherein the E3 ubiquitin ligase ligand is a CBRN or VHL ligase ligand.
B68 to B138. In embodiments B68 to B138, the compound for use of embodiment B67 is wherein the E3 ubiquitin ligase ligand is as disclosed in embodiments A40 to A110, respectively, or a pharmaceutically acceptable salt thereof. B139. In embodiment B139, the compound for use of any embodiments BIA to B138 is wherein Hy is cycloalkylene, arylene, heteroarylene, heterocyclylene, bicyclic heterocyclylene, spiro heterocyclylene, bridged heterocyclylene, or fused heterocyclylene, where each of the aforementioned rings is substituted with R^{a}, R^{b}, and R^{c} independently selected from hydrogen, deuterium, alkyl, halo, haloalkyl, alkoxy, hydroxy, and cyano; or a pharmaceutically acceptable salt thereof; or a pharmaceutically acceptable salt thereof.
B140 to B157. In embodiments B140 to B157, the compound for use of embodiment B139 is wherein Hy is as disclosed in embodiments A25-A39b, respectively, or a pharmaceutically acceptable salt thereof.

### Embodiment C

In embodiments C1 to C279, the present disclosure includes:
C1. In embodiment C1, provided is a compound of Formula (Ia) or a pharmaceutically acceptable salt thereof, as defined in the fourth embodiment of the Summary.
C2. In embodiment C2, the compound of embodiment C1, or a pharmaceutically acceptable salt thereof, is wherein R¹ is alkyl, alkenyl, alkynyl, cycloalkyl, halo, haloalkyl, haloalkoxy, alkoxy, aryloxy, cyano, or cycloalkyl where the cycloalkyl is substituted with one to three halo.
C3. In embodiment C3, the compound of embodiment C1 or C2, or a pharmaceutically acceptable salt thereof, is wherein R¹ is halo, haloalkyl, or haloalkoxy.
C4. In embodiment C4, the compound of any one of embodiments C1 to C3, or a pharmaceutically acceptable salt thereof, is wherein R¹ is halo.
C5. In embodiment C5, the compound of any one of embodiments C1 to C3, or a pharmaceutically acceptable salt thereof, is wherein R¹ is haloalkyl.
C6. In embodiment C6, the compound of any one of embodiments C1 to C3, or a pharmaceutically acceptable salt thereof, is wherein R¹ is haloalkoxy.
C7. In embodiment C7, the compound of any one of embodiments C1 to C6, or a pharmaceutically acceptable salt thereof, is wherein R¹ is chloro, bromo, fluoro, difluoromethyl, trifluoromethyl, difluoroethyl, trifluoroethyl, difluoromethoxy, trifluoromethoxy, difluoroethoxy, or trifluoroethoxy.
C8. In embodiment C8, the compound of any one of embodiments C1 to C7, or a pharmaceutically acceptable salt thereof, is wherein R¹ is chloro, bromo, difluoromethyl, trifluoromethyl, difluoromethoxy, or trifluoromethoxy.
C9. In embodiment C9, the compound of any one of embodiments C1 to C4, C7, and C8, or a pharmaceutically acceptable salt thereof, is wherein R¹ is chloro or bromo.
C10. In embodiment C10, the compound of any one of embodiments C1 to C3, C5, C7, and C8, or a pharmaceutically acceptable salt thereof, is wherein R¹ is difluoromethyl or trifluoromethyl.
C11. In embodiment C11, the compound of any one of embodiments C1 to C3, C5, C7, C8, and C10, or a pharmaceutically acceptable salt thereof, is wherein R¹ is trifluoromethyl.
C12. In embodiment C12, the compound of embodiment C1 or C2, or a pharmaceutically acceptable salt thereof, is wherein R¹ is alkyl, alkenyl, or alkynyl.
C13. In embodiment C13, the compound of embodiment C1, C2, or C12, or a pharmaceutically acceptable salt thereof, is wherein R¹ is methyl, ethyl, propyl, vinyl, propenyl, ethynyl, or propynyl.
C14. In embodiment C14, the compound of embodiment C1, C2, C12, or C13, or a pharmaceutically acceptable salt thereof, is wherein R¹ is methyl, ethyl, or propyl.
C15. In embodiment C15, the compound of embodiment C1, C2, C12, or C13, or a pharmaceutically acceptable salt thereof, is wherein R¹ is vinyl, propenyl, ethynyl, or propynyl.
C16. In embodiment C16, the compound of embodiment C1 or C2, or a pharmaceutically acceptable salt thereof, is wherein R¹ is alkoxy.
C17. In embodiment 17, the compound of embodiment C1, C2, or C16, or a pharmaceutically acceptable salt thereof, is wherein R¹ is methoxy, ethoxy, or propoxy.
C18. In embodiment C18, the compound of embodiment C1 or C2, or a pharmaceutically acceptable salt thereof, is wherein R¹ is aryloxy, for example phenoxy.
C19. In embodiment C19, the compound of embodiment C1 or C2, or a pharmaceutically acceptable salt thereof, is wherein R¹ is cyano.
C20. In embodiment C20, the compound of embodiment C1 or C2, or a pharmaceutically acceptable salt thereof, is wherein R¹ is cycloalkyl.
C21. In embodiment C21, the compound of embodiment C1, C2, or C20, or a pharmaceutically acceptable salt thereof, is wherein R¹ is cyclopropyl.
C22. In embodiment C22, the compound of embodiment C1 or C2, or a pharmaceutically acceptable salt thereof, is wherein R¹ is cycloalkyl substituted with one to three halo which are independently selected (for example fluorocyclopropyl or difluorocyclopropyl).
C23. In embodiment C23, the compound of embodiment C1, or a pharmaceutically acceptable salt thereof, is wherein R¹ is alkylthio, pentafluorothio, haloalkylthio, amino, alkylamino, dialkylamino, cycloalkyl, cycloalkoxy, cycloalkylalkyl, bridged cycloalkyl, bridged cycloalkoxy, bridged cycloalkylalkyl, cyanoalkyl, cyanoalkoxy, alkoxyalkyl, aminoalkyl, aminoalkoxy, alkylaminoalkyl, dialkylaminoalkyl, alkylaminoalkoxy, dialkylaminoalkoxy, acyl, azidocarbonyl, alkoxycarbonyl, alkylcarbonylamino, aminocarbonyl, alkylaminocarbonyl, dialkylaminocarbonyl, aminosulfonyl, alkylaminosulfonyl, dialkylaminosulfonyl, substituted sulfonyl, substituted sulfinyl, substituted ureido, aryl, aralkyl, aryloxy, heteroaryl, heteroaralkyl, heteroaryloxy, heterocyclyl, heterocyclylalkyl, heterocyclyloxy, fused heterocyclyl, fused heterocyclyloxy, or fused heterocyclylalkyl, wherein cycloalkyl, by itself or as part of cycloalkoxy and cycloalkylalkyl, aryl, by itself or as part of aralkyl and aryloxy, heteroaryl, by itself or as part of heteroaralkyl and heteroaryloxy, heterocyclyl, by itself or as part of heterocyclylalkyl and heterocyclyloxy, bridged cycloalkyl, alone or as part of bridged cycloalkoxy and bridged cycloalkylalkyl, and fused heterocyclyl, by itself or as part of fused heterocyclylalkyl and fused heterocyclyloxy, are substituted with one, two, or three substituents independently selected from hydrogen, alkyl, alkoxy, halo, haloalkyl, haloalkoxy, and cyano.
C24. In embodiment C24, the compound of embodiment C1 or C23, or a pharmaceutically acceptable salt thereof, is wherein R¹ is aryl, heteroaryl, heterocyclyl, cyanoalkyl, alkoxyalkyl, aminoalkyl, alkylaminoalkyl, dialkylaminoalkyl, heteroaryloxy, cyanoalkoxy, alkylthio, amino, alkylamino, dialkylamino, —SCF₃, —SF₅, fused heterocyclyl, bridged cycloalkyl, cycloalkylalkyl, heterocyclylalkyl, aralkyl, aminoalkoxy, alkoxycarbonyl, alkylcarbonylamino, acyl, azidocarbonyl, aminocarbonyl, alkylaminocarbonyl, dialkylaminocarbonyl, substituted ureido, aminosulfonyl, alkylaminosulfonyl, dialkylaminosulfonyl, and substituted sulfonyl, wherein aryl, alone and in arylalkyl, heteroaryl, alone and in heteroaryloxy, heterocyclyl, fused heterocyclyl, bridged cycloalkyl, alone and in cycloalkylalkyl, and heterocyclyl, alone and in heterocyclylalkyl, are substituted with one, two, or three substituents independently selected from hydrogen, alkyl, alkoxy, halo, haloalkyl, haloalkoxy, and cyano.
C25. In embodiment C25, the compound of embodiment C1, C23, or C24, or a pharmaceutically acceptable salt thereof, is wherein R¹ is phenyl, pyrazolyl, imidazolyl, oxazolyl, thiazolyl, triazolyl, tetrahydrofuranyl, tetrahydropyranyl, pyrrolidinyl, piperidinyl, morpholinyl, thiomorpholinyl, piperazinyl, cyanomethyl, cyanoethyl, methoxymethyl, aminomethyl, methylaminomethyl, dimethylaminomethyl, 2,3-dihydrobenzofuranyl, benzodihydropyranyl, 1,4-benzodioxanyl, 2,3-dihydrofuro[3,2-c]pyridine, 2,3-dihydrofuro[2,3-c]pyridine, or 1,2,3,4-tetrahydroquinolinyl; wherein each of the rings is substituted with hydrogen, alkyl, alkoxy, halo, haloalkyl, haloalkoxy, or cyano.
C26. In embodiment C26, the compound of embodiment C1 or C23, or a pharmaceutically acceptable salt thereof, is wherein R¹ is aryl, heteroaryl, heterocyclyl, cyanoalkyl, alkoxyalkyl, aminoalkyl, alkylaminoalkyl, dialkylaminoalkyl, heteroaryloxy, cyanoalkoxy, alkylthio, amino, alkylamino, dialkylamino, —SCF₃, or —SF₅; wherein each of the rings is substituted with hydrogen, alkyl, alkoxy, halo, haloalkyl, haloalkoxy, or cyano.
C27. In embodiment C27, the compound of embodiment C1, C23, or C26, or a pharmaceutically acceptable salt thereof, is wherein R¹ is phenyl (substituted with hydrogen, alkyl, alkoxy, halo, cyano, haloalkyl, or haloalkoxy), pyrazolyl, imidazolyl, oxazolyl, thiazolyl, triazolyl (substituted with hydrogen or alkyl), tetrahydrofuranyl, tetrahydropyranyl, pyrrolidinyl, piperidinyl, morpholinyl, thiomorpholinyl, piperazinyl (substituted with hydrogen, alkyl, alkoxy, halo, haloalkyl, haloalkoxy, or cyano) cyanomethyl, cyanoethyl, methoxymethyl, aminomethyl, methylaminomethyl, or dimethylaminomethyl.
C28. In embodiment C28, the compound of embodiment C1 or C23, or a pharmaceutically acceptable salt thereof, is wherein R¹ is alkylthio (e.g., methylthio).
C29. In embodiment C29, the compound of embodiment C1 or C23, or a pharmaceutically acceptable salt thereof, is wherein R¹ is pentafluorothio.
C30. In embodiment C30, the compound of embodiment C1 or C23, or a pharmaceutically acceptable salt thereof, is wherein R¹ is haloalkylthio (e.g., trifluoromethylthio).
C31. In embodiment C31, the compound of embodiment C1 or C23, or a pharmaceutically acceptable salt thereof, is wherein R¹ is amino.
C32. In embodiment C32, the compound of embodiment C1 or C23, or a pharmaceutically acceptable salt thereof, is wherein R¹ is alkylamino (e.g, methylamino).
C33. In embodiment C33, the compound of embodiment C1 or C23, or a pharmaceutically acceptable salt thereof, is wherein R¹ is dialkylamino (e.g., dimethylamino).
C34. In embodiment C34, the compound of embodiment C1 or C23, or a pharmaceutically acceptable salt thereof, is wherein R¹ is cycloalkyl substituted with one, two, or three substituents independently selected from hydrogen, alkyl, alkoxy, halo, haloalkyl, haloalkoxy, and cyano.
C35. In embodiment C35, the compound of embodiment C1, C23 or C34, or a pharmaceutically acceptable salt thereof, is wherein R¹ is cyclopropyl, cyclobutyl, or cyclopentyl, each ring substituted with one or two substituents independently selected from hydrogen, methyl, fluoro, and cyano.
C36. In embodiment C36, the compound of embodiment C1 or C23, or a pharmaceutically acceptable salt thereof, is wherein R¹ is cycloalkoxy where the cycloalkyl is substituted with one, two, or three substituents independently selected from hydrogen, alkyl, alkoxy, halo, haloalkyl, haloalkoxy, and cyano.
C37. In embodiment C37, the compound of embodiment C1, C23, or C36, or a pharmaceutically acceptable salt thereof, is wherein R¹ is cyclopropyloxy, cyclobutyloxy, or cyclopentyloxy, each cycloalkyl ring of cycloalkyloxy substituted with one or two substituents independently selected from hydrogen, methyl, fluoro, and cyano.
C38. In embodiment C38, the compound of embodiment C1 or C23, or a pharmaceutically acceptable salt thereof, is wherein R¹ is cycloalkylalkyl where the cycloalkyl is substituted with one, two, or three substituents independently selected from hydrogen, alkyl, alkoxy, halo, haloalkyl, haloalkoxy, and cyano.
C39. In embodiment C39, the compound of embodiment C1, C23, or C38, or a pharmaceutically acceptable salt thereof, is wherein R¹ is cyclopropylmethyl, cyclobutylmethyl, or cyclopentylmethyl, the ring of cycloalkylalkyl substituted with one or two substituents independently selected from hydrogen, methyl, fluoro, and cyano.
C40. In embodiment C40, the compound of embodiment C1 or C23, or a pharmaceutically acceptable salt thereof, is wherein R¹ is bridged cycloalkyl (such as bicyclo[1.1.1]pent-1-yl or bicyclo[2.2.1]heptyl) where the bridged cycloalkyl is substituted with one, two, or three substituents independently selected from hydrogen, alkyl, alkoxy, halo, haloalkyl, haloalkoxy, and cyano.
C41. In embodiment C41, the compound of embodiment C1, C23, or C40, or a pharmaceutically acceptable salt thereof, is wherein R¹ is bridged cycloalkyl (such as bicyclo[1.1.1]pent-1-yl or bicyclo[2.2.1]heptyl) where the bridged cycloalkyl is substituted with one or two substituents independently selected from hydrogen, methyl, fluoro, and cyano.
C42. In embodiment C42, the compound of embodiment C1 or C23, or a pharmaceutically acceptable salt thereof, is wherein R¹ is bridged cycloalkoxy (such as bicyclo[1.1.1]pent-1-yloxy or bicyclo[2.2.1]heptyloxy) where the bridged cycloalkyl of bridged cycloalkoxy is substituted with one, two, or three substituents independently selected from hydrogen, alkyl, alkoxy, halo, haloalkyl, haloalkoxy, and cyano.
C43. In embodiment C43, the compound of embodiment C1, C23, or C42, or a pharmaceutically acceptable salt thereof, is wherein R¹ is bridged cycloalkoxy (such as bicyclo[1.1.1]pent-1-yloxy or bicyclo[2.2.1]heptyloxy) where the bridged cycloalkyl of bridged cycloalkoxy is substituted with one or two substituents independently selected from hydrogen, methyl, fluoro, and cyano.
C44. In embodiment C44, the compound of embodiment C1 or C23, or a pharmaceutically acceptable salt thereof, is wherein R¹ is bridged cycloalkylalkyl (such as bicyclo[1.1.1]pent-1-ylmethyl or bicyclo[2.2.1]heptylmethyl) where the bridged cycloalkyl of bridged cyclylalkylalkyl is substituted with one, two, or three substituents independently selected from hydrogen, alkyl, alkoxy, halo, haloalkyl, haloalkoxy, and cyano.
C45. In embodiment C45, the compound of embodiment C1, C23, or C44, or a pharmaceutically acceptable salt thereof, is wherein R¹ is bridged cycloalkylalkyl (such as bicyclo[1.1.1]pent-1-ylmethyl or bicyclo[2.2.1]heptylmethyl) where the bridged cycloalkyl of bridged cyclylalkylalkyl is substituted with one or two substituents independently selected from hydrogen, methyl, fluoro, and cyano.
C46. In embodiment C46, the compound of embodiment C1 or C23, or a pharmaceutically acceptable salt thereof, is wherein R¹ is cyanoalkyl, such as cyanomethyl or cyanoethyl,
C47. In embodiment C47, the compound of embodiment C1 or C23, or a pharmaceutically acceptable salt thereof, is wherein R¹ is cyanoalkoxy (such as cyanomethoxy or cyanoethoxy).
C48. In embodiment C48, the compound of embodiment C1 or C23, or a pharmaceutically acceptable salt thereof, is wherein R¹ is alkoxyalkyl (e.g., methoxymethyl).
C49. In embodiment C49, the compound of embodiment C1 or C23, or a pharmaceutically acceptable salt thereof, is wherein R¹ is aminoalkyl (e.g., aminomethyl).
C50. In embodiment C50, the compound of embodiment C1 or C23, or a pharmaceutically acceptable salt thereof, is wherein R¹ is aminoalkoxy e.g., R¹ is aminomethyloxy.
C51. In embodiment C51, the compound of embodiment C1 or C23, or a pharmaceutically acceptable salt thereof, is wherein R¹ is alkylaminoalkyl (e.g., R¹ is methylaminomethyl).
C52. In embodiment C52, the compound of embodiment C1 or C23, or a pharmaceutically acceptable salt thereof, is wherein R¹ is dialkylaminoalkyl (e.g., R¹ is dimethylaminomethyl).
C53. In embodiment C53, the compound of embodiment C1 or C23, or a pharmaceutically acceptable salt thereof, is wherein R¹ is alkylaminoalkoxy (e.g., R¹ is methylaminomethyloxy).
C54. In embodiment C54, the compound of embodiment C1 or C23, or a pharmaceutically acceptable salt thereof, is wherein R¹ is dialkylaminoalkoxy (e . . . g, R¹ is dimethylaminomethyloxy).
C55. In embodiment C55 the compound of embodiment C1 or C23, or a pharmaceutically acceptable salt thereof, is wherein R¹ is acyl.
C55a. In embodiment C55a, the compound of embodiment C1, C23, or C55, or a pharmaceutically acceptable salt thereof, is wherein R¹ is alkylcarbonyl (such as methylcarbonyl).

C56. In embodiment C56, the compound of embodiment C1 or C23, or a pharmaceutically acceptable salt thereof, is wherein R¹ is azidocarbonyl.
C57. In embodiment C57, the compound of embodiment C1 or C23, or a pharmaceutically acceptable salt thereof, is wherein R¹ is alkoxycarbonyl (e.g., methoxycarbonyl or ethoxycarbonyl).
C58. In embodiment C58, the compound of embodiment C1 or C23, or a pharmaceutically acceptable salt thereof, is wherein R¹ is alkylcarbonylamino.

C59. In embodiment C59, the compound of embodiment C1 or C23, or a pharmaceutically acceptable salt thereof, is wherein R¹ is aminocarbonyl.
C60. In embodiment C60, the compound of embodiment C1 or C23, or a pharmaceutically acceptable salt thereof, is wherein R¹ is alkylaminocarbonyl (such as methylaminocarbonyl).
C61. In embodiment C61, the compound of embodiment C1 or C23, or a pharmaceutically acceptable salt thereof, is wherein R¹ is dialkylaminocarbonyl (such as dimethylaminocarbonyl).
C62. In embodiment C62, the compound of embodiment C1 or C23, or a pharmaceutically acceptable salt thereof, is wherein R¹ is aminosulfonyl.
C63. In embodiment C63, the compound of embodiment C1 or C23, or a pharmaceutically acceptable salt thereof, is wherein R¹ is alkylaminosulfonyl (such as methylaminosulfonyl).
C64. In embodiment C64, the compound of embodiment C1 or C23, or a pharmaceutically acceptable salt thereof, is wherein R¹ is dialkylaminosulfonyl (such as dimethylaminosulfonyl).
C65. In embodiment C65, the compound of embodiment C1 or C23, or a pharmaceutically acceptable salt thereof, is wherein R¹ is substituted sulfonyl (e.g, methylsulfonyl).
C66. In embodiment C66, the compound of embodiment C1 or C23, or a pharmaceutically acceptable salt thereof, is wherein R¹ is substituted sulfinyl.
C67. In embodiment C67, the compound of embodiment C1 or C23, or a pharmaceutically acceptable salt thereof, is wherein R¹ is substituted ureido.
C68. In embodiment C68, the compound of embodiment C1 or C23, or a pharmaceutically acceptable salt thereof, is wherein R¹ is aryl substituted with one, two, or three substituents independently selected from hydrogen, alkyl, alkoxy, halo, haloalkyl, haloalkoxy, and cyano.
C69. In embodiment C69, the compound of embodiment C1, C23, or C68, or a pharmaceutically acceptable salt thereof, is wherein R¹ is phenyl substituted with one, two, or three substituents independently selected from hydrogen, alkyl, alkoxy, halo, haloalkyl, haloalkoxy, and cyano.
C69a. In embodiment C69a, the compound of embodiment C1, C23, C68, or C69, or a pharmaceutically acceptable salt thereof, is wherein R¹ is phenyl substituted with one, two, or three substituents independently selected from hydrogen, methyl, fluoro, cyano, difluoromethyl, trifluoromethyl, difluoromethoxy, and trifluoromethoxy.
C70. In embodiment C70, the compound of embodiment C1 or C23, or a pharmaceutically acceptable salt thereof, is wherein R¹ is aralkyl (such as benzyl) where the aryl is substituted with one, two, or three substituents independently selected from hydrogen, alkyl, alkoxy, halo, haloalkyl, haloalkoxy, and cyano.
C71. In embodiment C71, the compound of embodiment C1, C23, or C70, or a pharmaceutically acceptable salt thereof, is wherein R¹ is benzyl where phenyl of benzyl is substituted with one, two, or three substituents independently selected from hydrogen, methyl, fluoro, cyano, difluoromethyl, trifluoromethyl, difluoromethoxy, and trifluoromethoxy.
C72. In embodiment C72, the compound of embodiment C1 or C23, or a pharmaceutically acceptable salt thereof, is wherein R¹ is heteroaryl where the heteroaryl is substituted with one, two, or three substituents independently selected from hydrogen, alkyl, alkoxy, halo, haloalkyl, haloalkoxy, and cyano.
C73. In embodiment C73, the compound of embodiment C1, C23, or C72, or a pharmaceutically acceptable salt thereof, is wherein R¹ is pyrazolyl, imidazolyl, oxazolyl, thiazolyl, or triazolyl, each of which is substituted with one, two, or three substituents independently selected from hydrogen, alkyl, alkoxy, halo, haloalkyl, haloalkoxy, and cyano.
C73a. In embodiment C73a, the compound of embodiment C1, C23, or C72, or a pharmaceutically acceptable salt thereof, is wherein R¹ is pyrazolyl, imidazolyl, oxazolyl, thiazolyl, or triazolyl, each of which is substituted with one substituent selected from hydrogen and alkyl.
C74. In embodiment C74, the compound of embodiment C1 or C23, or a pharmaceutically acceptable salt thereof, is wherein R¹ is heteroaralkyl where the heteroaryl of heteroaralkyl is substituted with one, two, or three substituents independently selected from hydrogen, alkyl, alkoxy, halo, haloalkyl, haloalkoxy, and cyano.
C74a. In embodiment C74a, the compound of embodiment C1, C23, or C74, or a pharmaceutically acceptable salt thereof, is wherein heteroaryl of heteroaralkyl of R¹ is pyrazolyl, imidazolyl, oxazolyl, thiazolyl, or triazolyl, each of which is substituted with one, two, or three substituents independently selected from hydrogen, alkyl, alkoxy, halo, haloalkyl, haloalkoxy, and cyano.
C75. In embodiment C75, the compound of embodiment C1 or C23, or a pharmaceutically acceptable salt thereof, is wherein R¹ is heteroaryloxy where the heteroaryl is substituted with one, two, or three substituents independently selected from hydrogen, alkyl, alkoxy, halo, haloalkyl, haloalkoxy, and cyano.
C76. In embodiment C76, the compound of embodiment C1, C23, or C75, or a pharmaceutically acceptable salt thereof, is wherein the heteroaryl of heteroaryloxy of R¹ is pyrazolyl, imidazolyl, oxazolyl, thiazolyl, or triazolyl, each of which is substituted with one, two, or three substituents independently selected from hydrogen, alkyl, alkoxy, halo, haloalkyl, haloalkoxy, and cyano.
C77. In embodiment C77, the compound of embodiment C1 or C23, or a pharmaceutically acceptable salt thereof, is wherein R¹ is heterocyclyl where the heterocyclyl is substituted with one, two, or three substituents independently selected from hydrogen, alkyl, alkoxy, halo, haloalkyl, haloalkoxy, and cyano.
C78. In embodiment C78, the compound of embodiment C1, C23, or C77, or a pharmaceutically acceptable salt thereof, is wherein R¹ is tetrahydrofuranyl, tetrahydropyranyl, pyrrolidinyl, piperidinyl, morpholinyl, thiomorpholinyl, or piperazinyl where the piperazinyl is optionally substituted with alkyl, alkoxy, halo, haloalkyl, haloalkoxy, or cyano.
C79. In embodiment C79, the compound of embodiment C1 or C23, or a pharmaceutically acceptable salt thereof, is wherein R¹ is heterocyclylalkyl where the heterocyclyl is substituted with one, two, or three substituents independently selected from hydrogen, alkyl, alkoxy, halo, haloalkyl, haloalkoxy, and cyano.
C80. In embodiment C80, the compound of embodiment C1, C23, or C79, or a pharmaceutically acceptable salt thereof, is wherein the heterocyclyl of heterocyclylalkyl of R¹ is tetrahydrofuranyl, tetrahydropyranyl, pyrrolidinyl, piperidinyl, morpholinyl, thiomorpholinyl, or piperazinyl where the piperazinyl is optionally substituted with alkyl, alkoxy, halo, haloalkyl, haloalkoxy, or cyano.
C81. In embodiment C81, the compound of embodiment C1 or C23, or a pharmaceutically acceptable salt thereof, is wherein R¹ is heterocyclyloxy where the heterocyclyl is substituted with one, two, or three substituents independently selected from hydrogen, alkyl, alkoxy, halo, haloalkyl, haloalkoxy, and cyano.
C82. In embodiment C82, the compound of embodiment C1, C23 or C81, or a pharmaceutically acceptable salt thereof, is wherein the heterocyclyl of heterocyclyloxy of R¹ is tetrahydrofuranyl, tetrahydropyranyl, pyrrolidinyl, piperidinyl, morpholinyl, thiomorpholinyl, or piperazinyl where the piperazinyl is optionally substituted with alkyl, alkoxy, halo, haloalkyl, haloalkoxy, or cyano.
C83. In embodiment C83, the compound of embodiment C1 or C23, or a pharmaceutically acceptable salt thereof, is wherein R¹ is fused heterocyclyl substituted with one, two, or three substituents independently selected from hydrogen, alkyl, alkoxy, halo, haloalkyl, haloalkoxy, and cyano.
C84. In embodiment C84, the compound of embodiment C1, C23, or C83, or a pharmaceutically acceptable salt thereof, is wherein R¹ is fused heterocyclyl selected from 2,3-dihydrobenzofuranyl, benzodihydropyranyl, 1,4-benzodioxanyl, 2,3-dihydrofuro[3,2-c]pyridine, 2,3-dihydrofuro[2,3-c]pyridine, and 1,2,3,4-tetrahydroquinolinyl, each of which is substituted with one, two, or three substituents independently selected from hydrogen, alkyl, alkoxy, halo, haloalkyl, haloalkoxy, and cyano.
C85. In embodiment C85, the compound of embodiment C1 or C23, or a pharmaceutically acceptable salt thereof, is wherein R¹ is fused heterocyclyloxy where the heterocyclyl is substituted with one, two, or three substituents independently selected from hydrogen, alkyl, alkoxy, halo, haloalkyl, haloalkoxy, and cyano.
C86. In embodiment C86, the compound of embodiment C1 or C23, or a pharmaceutically acceptable salt thereof, is wherein R¹ is fused heterocyclylalkyl where the heterocyclyl is substituted with one, two, or three substituents independently selected from hydrogen, alkyl, alkoxy, halo, haloalkyl, haloalkoxy, and cyano.
C87. In embodiment C87, the compound of embodiment C1 and C23 to C86, or a pharmaceutically acceptable salt thereof, is wherein R¹ is selected from:

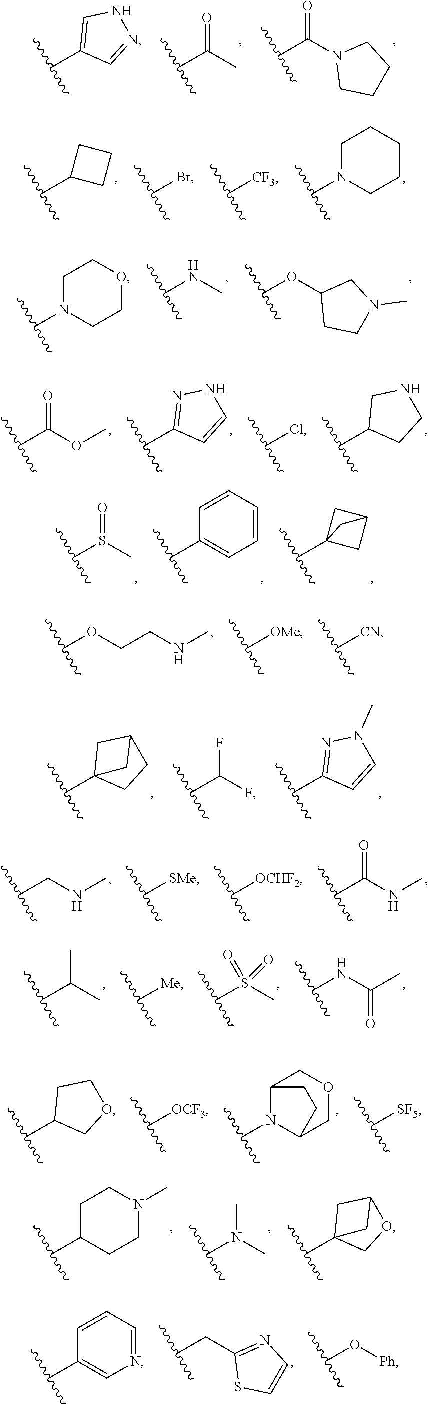

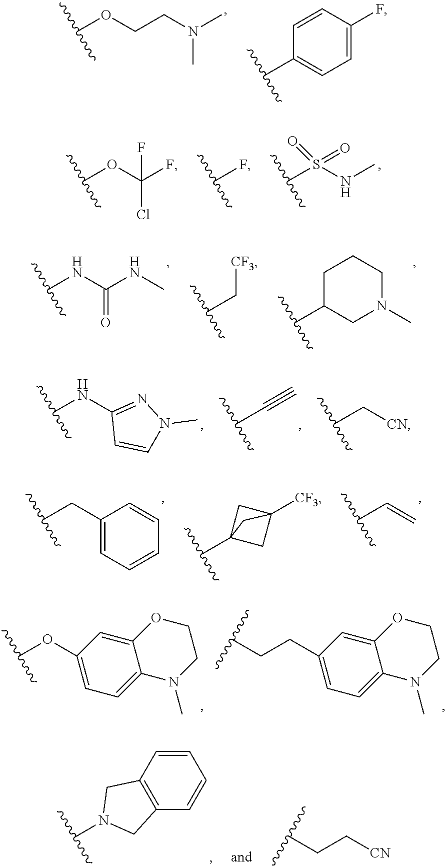

and isomers thereof (R and/or S isomers, and/or geometric isomers).
C88. In embodiment C88, the compound of any one of embodiments C1 to C87, or a pharmaceutically acceptable salt thereof, is wherein R² and R^{2a} are hydrogen.
C89. In embodiment C89, the compound of any one of embodiments C1 to C87, or a pharmaceutically acceptable salt thereof, is wherein one of R² and R^{2a} is deuterium and the other of R² and R^{2a} is hydrogen or both R² and R^{2a} are deuterium.
C90. In embodiment C90, the compound of any one of embodiments C1 to C89, or a pharmaceutically acceptable salt thereof, is wherein Hy is heterocyclylene, phenylene, spiro heterocyclylene, bridged heterocyclylene, or cycloalkylene, wherein each of the aforementioned rings is substituted with R^{a}, R^{b}, and R^{c} where R^{a} and R^{b} are independently selected from hydrogen, deuterium, alkyl, halo, haloalkyl, alkoxy, hydroxy, and cyano, and R^{c} is hydrogen.
C91. In embodiment C91, the compound of any one of embodiments C1 to C90, or a pharmaceutically acceptable salt thereof, is wherein Hy is heterocyclylene substituted with R^{a}, R^{b}, and R^{c} where R^{a} and R^{b} are independently selected from hydrogen, deuterium, alkyl, halo, haloalkyl, alkoxy, hydroxy, and cyano, and R^{b} is hydrogen.
C92. In embodiment C92, the compound of any one of embodiments C1 to C91, or a pharmaceutically acceptable salt thereof, is wherein the heterocyclylene of Hy is pyrrolidin-1,3-diyl or piperidin-1,4-diyl, where Hy is substituted with R^{a}, R^{b}, and R^{c} where R^{a} and R^{b} are independently hydrogen, deuterium, methyl, fluoro, methoxy, or hydroxy, R^{c} is hydrogen, and L is attached to the nitrogen atom of the piperidin-1,4-diyl or pyrrolidin-1,3-diyl ring of Hy.
C93. In embodiment C93, the compound of any one of embodiments C1 to C92, or a pharmaceutically acceptable salt thereof, is wherein the heterocyclylene of Hy is:

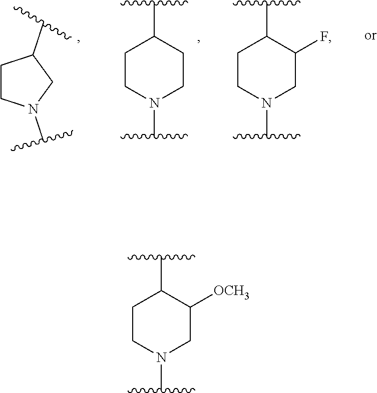 [SMILES: *C1CCN(*)C1 ; *C1CCN(*)CC1 ; *C1CCN(*)CC1F ; "or" ; *C1CCN(*)CC1OC]

where the N atom of the pyrrolidin-1,3-diyl or piperidin-1,4-diyl rings is attached to L.
C94. In embodiment C94, the compound of any one of C1 to C93, or a pharmaceutically acceptable salt thereof, is wherein the heterocyclylene of Hy is:

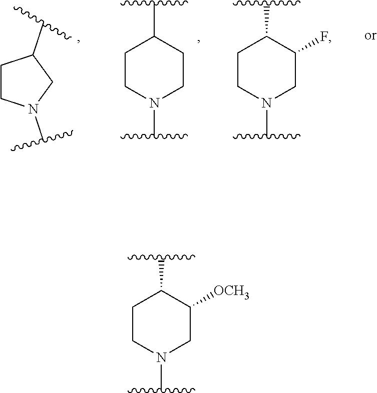 [SMILES: *C1CCN(*)C1 ; *C1CCN(*)CC1 ; *[C@H]1CCN(*)C[C@H]1F ; "or" ; *[C@H]1CCN(*)C[C@H]1OC]

where the N atom of the pyrrolidin-1,3-diyl or piperidin-1,4-diyl rings is attached to L.
C95. In embodiment C95, the compound of any one of embodiments C1 to C94, or a pharmaceutically acceptable salt thereof, is wherein the heterocyclylene of Hy is:

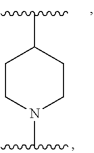 [SMILES: *C1CCN(*)CC1]

where the N atom of the piperidin-1,4-diyl ring is attached to L.
C96. In embodiment C96, the compound of any one of C1 to C90, or a pharmaceutically acceptable salt thereof, is wherein Hy is bridged heterocyclylene substituted with R^{a}, R^{b}, and R^{c} independently selected from hydrogen, deuterium, alkyl, halo, haloalkyl, alkoxy, hydroxy, and cyano.
C97. In embodiment C97, the compound of any one of embodiments C1 to C90, and C96, or a pharmaceutically acceptable salt thereof, is wherein the bridged heterocyclylene of Hy is a ring of formula:

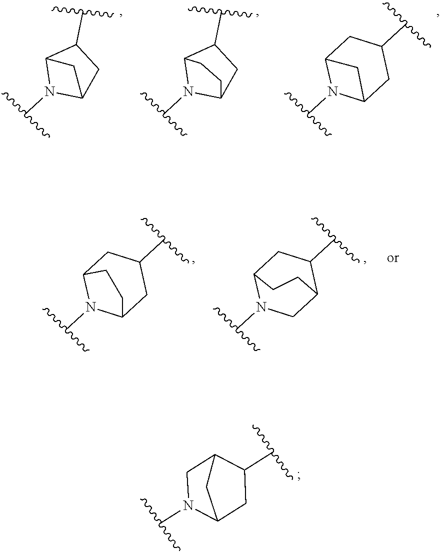 [SMILES: *C1CC2CC1N2* ; *C1CC2CCC1N2* ; *C1CC2CC(C1)N2* ; *C1CC2CCC(C1)N2* ; *C1CC2CCC1CN2* ; *C1CC2CC1CN2*]

and each ring is substituted with R^{a}, R^{b}, and R^{c} where R^{c} is hydrogen, and L is attached to the nitrogen atom of each ring.
C98. In embodiment C98, the compound of embodiment C96 or C97, or a pharmaceutically acceptable salt thereof, is wherein R^{a} and R^{b} are independently hydrogen, deuterium, methyl, fluoro, methoxy, or hydroxy.
C99. In embodiment C99, the compound of embodiment C96, C97, or C98, or a pharmaceutically acceptable salt thereof, is wherein R^{b} is hydrogen.
C100. In embodiment C100, the compound of any one of embodiments C1 to C90, or a pharmaceutically acceptable salt thereof, is wherein Hy is cycloalkylene substituted with R^{a}, R^{b}, and R^{c} where R^{a} is deuterium, methyl, fluoro, methoxy, or hydroxy and R^{b} and R^{c} are hydrogen.
C101. In embodiment C101, the compound of any one of embodiments C1 to C90, and C100, or a pharmaceutically acceptable salt thereof, is wherein the cycloalkylene of Hy is cyclohexylene.
C102. In embodiment C102, the compound of any one of embodiments C1 to C90, C100, and C101, or a pharmaceutically acceptable salt thereof, is wherein the cycloalkylene of Hy is

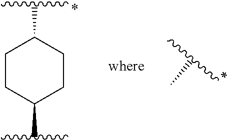

denotes bond to NH and [symbol] denotes bond of L.
C103. In embodiment C103, the compound of any one of embodiments C1 to C90, or a pharmaceutically acceptable salt thereof, is wherein Hy is arylene wherein the arylene is phenylene substituted with R^{a}, R^{b}, and R^{c} where R^{a} and R^{b} are independently selected from hydrogen, deuterium, alkyl, halo, haloalkyl, alkoxy, hydroxy, and cyano, and R^{c} is hydrogen.
C104. In embodiment C104, the compound of any one of embodiments C1 to C90, or a pharmaceutically acceptable salt thereof, is wherein Hy is spiro heterocyclylene substituted (e.g., 2-azaspiro[3.3]heptan-2-yl) with R^{a}, R^{b}, and R^{c} where R^{a} and R^{b} are independently selected from hydrogen, deuterium, alkyl, halo, haloalkyl, alkoxy, hydroxy, and cyano, and R^{c} is hydrogen.
C105. In embodiment C105, the compound of any one of embodiments C1 to C90 and C103, or a pharmaceutically acceptable salt thereof, is wherein the phenylene of Hy is 1,4-phenylene according to structure

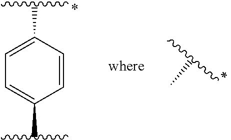

denotes bond to NH and [symbol] denotes bond of L.
C106. In embodiment C106, the compound of any one of embodiments C1 to C89, or a pharmaceutically acceptable salt thereof, is wherein Hy is fused heterocyclylene substituted with R^{a}, R^{b}, and R^{c} where R^{a} and R^{b} are independently selected from hydrogen, deuterium, alkyl, halo, haloalkyl, alkoxy, hydroxy, and cyano, and R^{c} is hydrogen.
C107. In embodiment C107, the compound of any one of embodiments C1 to C89, or a pharmaceutically acceptable salt thereof, is wherein Hy is bicyclic heterocyclylene substituted with R^{a}, R^{b}, and R^{c} where R^{a} and R^{b} are independently selected from hydrogen, deuterium, alkyl, halo, haloalkyl, alkoxy, hydroxy, and cyano, and R^{c} is hydrogen.
C108. In embodiment C108, the compound of any one of embodiments C1 to C107, or a pharmaceutically acceptable salt thereof, is wherein the Degron is an E3 ubiquitin ligase ligand of formula (i) or (ii).
C109. In embodiment C109, the compound of any one of embodiments C1 to C108, or a pharmaceutically acceptable salt thereof, is wherein the Degron is an E3 ubiquitin ligase ligand of formula (i):

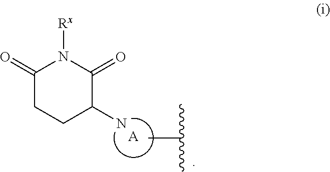

C110. In embodiment C110, the compound of any one of embodiments C1 to C109, or a pharmaceutically acceptable salt thereof, is wherein the ring A of the E3 ubiquitin ligase ligand of formula (i) is a group of formula (a):

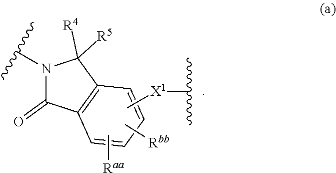

C111. In embodiment C111, the compound of any one of embodiments C1 to C110, or a pharmaceutically acceptable salt thereof, is wherein R⁴ and R⁵ are independently hydrogen or alkyl.
C112. In embodiment C112, the compound of any one of embodiments C1 to C111, or a pharmaceutically acceptable salt thereof, is wherein R⁴ and R⁵ are hydrogen.
C113. In embodiment C113, the compound of any one of embodiments C1 to C111, or a pharmaceutically acceptable salt thereof, is wherein R⁴ is hydrogen and R⁵ is methyl.
C114. In embodiment C114, the compound of any one of embodiments C1 to C110, or a pharmaceutically acceptable salt thereof, is wherein R⁴ and R⁵ together with the carbon to which they are attached form >C═O.
C115. In embodiment C115, the compound of any one of embodiments C1 to C109, or a pharmaceutically acceptable salt thereof, is wherein the ring A of the E3 ubiquitin ligase ligand of formula (i) is a group of formula (b):

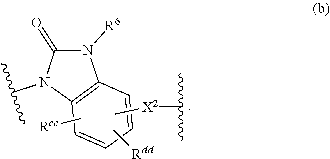

C116. In embodiment C116, the compound of any one of embodiments C1 to C109, and C115, or a pharmaceutically acceptable salt thereof, is wherein R⁶ is hydrogen.
C117. In embodiment C117, the compound of any one of embodiments C1 to C109, and C115, or a pharmaceutically acceptable salt thereof, wherein R⁶ is alkyl, e.g., methyl.
C118. In embodiment C118, the compound of any one of embodiments C1 to C109, or a pharmaceutically acceptable salt thereof, is wherein the ring A of the E3 ubiquitin ligase ligand of formula (i) is a group of formula (c):

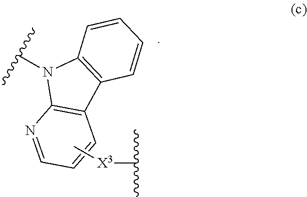

C119. In embodiment C119, the compound of any one of embodiments C1 to C117, or a pharmaceutically acceptable salt thereof, is wherein the ring A of the E3 ubiquitin ligase ligand of formula (i) is:

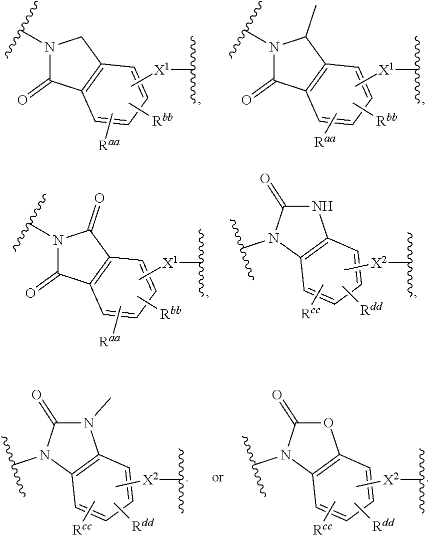

C120. In embodiment C120, the compound of any one of embodiments C1 to C117 and C119, or a pharmaceutically acceptable salt thereof, is wherein the ring A of the E3 ubiquitin ligase ligand of formula (i) is:

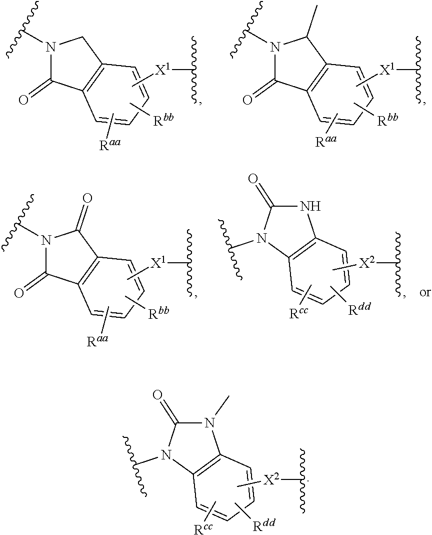

C121. In embodiment C121, the compound of any one of embodiments C1 to C117 and C119, or a pharmaceutically acceptable salt thereof, is wherein ring A of the E3 ubiquitin ligase ligand of formula (i) is:

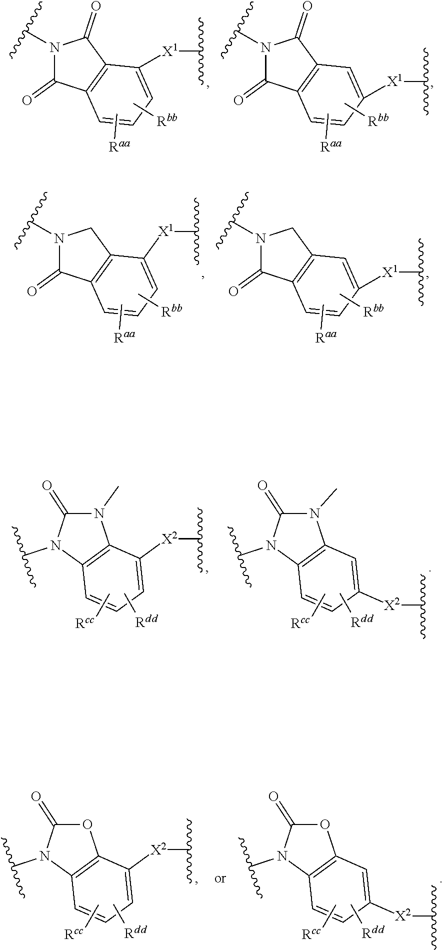

C122. In embodiment C122, the compound of any one of embodiments C1 to C117 and C119 to C121, or a pharmaceutically acceptable salt thereof, is wherein ring A of the E3 ubiquitin ligase ligand of formula (i) is:

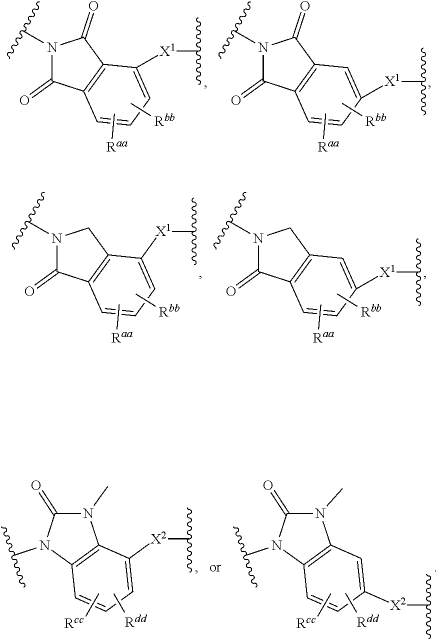

C123. In embodiment C123, the compound of any one of embodiments C1 to C117, C119, and C121, or a pharmaceutically acceptable salt thereof, is wherein ring A of the E3 ubiquitin ligase ligand of formula (i) is:

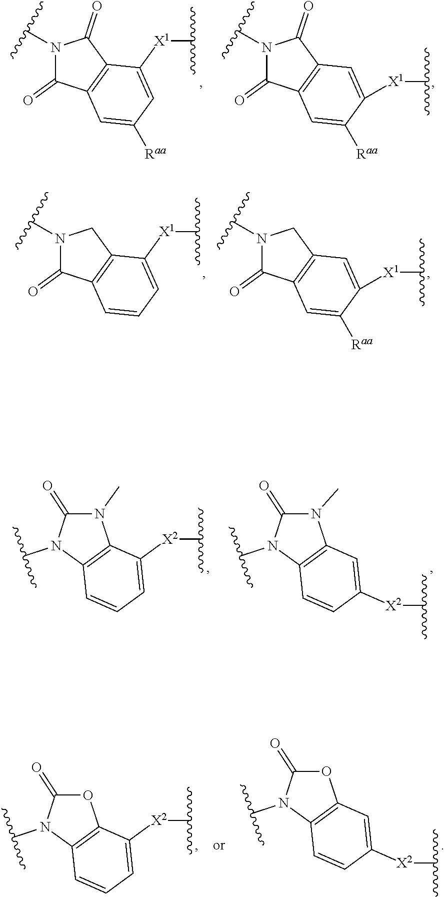

C124. In embodiment C124, the compound of any one of embodiments C1 to C117 and C119 to C122, or a pharmaceutically acceptable salt thereof, is wherein ring A of the E3 ubiquitin ligase ligand of formula (i) is:

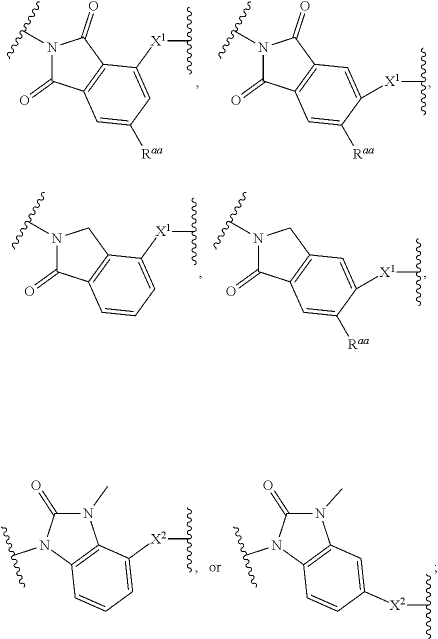

C125. In embodiment C125, the compound of any one of embodiments C1 to C110, C114, and C119 to C124, or a pharmaceutically acceptable salt thereof, is wherein ring A of the E3 ubiquitin ligase ligand of formula (i) is:

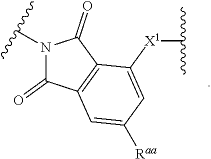

C126. In embodiment C126, the compound of any one of embodiments C1 to C110, C114, and C119 to C124, or a pharmaceutically acceptable salt thereof, is wherein ring A of the E3 ubiquitin ligase ligand of formula (i) is:

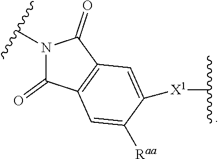

C127. In embodiment C127, the compound of any one of embodiments C1 to C112 and C119 to C124, or a pharmaceutically acceptable salt thereof, is wherein ring A of the E3 ubiquitin ligase ligand of formula (i) is:

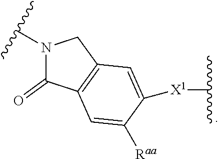

C128. In embodiment C128, the compound of any one of embodiments C1 to C112 and C119 to C124, or a pharmaceutically acceptable salt thereof, is wherein ring A of the E3 ubiquitin ligase ligand of formula (i) is:

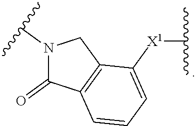

C129. In embodiment C129, the compound of any one of embodiments C1 to C109, C115, C117, and C119 to C124, or a pharmaceutically acceptable salt thereof, is wherein ring A of the E3 ubiquitin ligase ligand of formula (i) is:

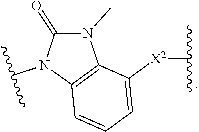

C130. In embodiment C130, the compound of any one of embodiments C1 to C109, C115, C117, and C119 to C124, or a pharmaceutically acceptable salt thereof, is wherein ring A of the E3 ubiquitin ligase ligand of formula (i) is:

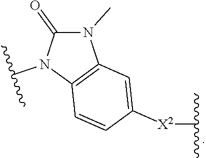

C131. In embodiment C131, the compound of any one of embodiments C1 to C117 and C119 to C127, or a pharmaceutically acceptable salt thereof, is wherein R^{aa}, R^{bb}, R^{cc}, and R^{dd} are independently selected from hydrogen, alkyl, alkoxy, halo, haloalkyl, and haloalkoxy. For sake of clarity, R^{aa}, R^{bb}, R^{cc}, and/or R^{dd} are hydrogen when they are not specifically drawn out in structures of formula (i) and (ii), respectively.
C132. In embodiment C132, the compound of any one of embodiments C1 to C117 and C119 to C127, or a pharmaceutically acceptable salt thereof, is wherein R^{aa}, R^{bb}, R^{cc}, and R^{dd} are independently selected from hydrogen, alkyl, alkoxy, halo, haloalkyl, and cyano.
C133. In embodiment C133, the compound of any one of embodiments C1 to C117, C119 to C127, C131, and C132, or a pharmaceutically acceptable salt thereof, is wherein R^{aa}, R^{bb}, R^{cc}, and R^{dd} are independently selected from hydrogen, methyl, methoxy, ethoxy, fluoro, trifluoromethyl, difluoromethyl, and trifluoromethoxy.
C134. In embodiment C134, the compound of any one of C1 to C117, C119 to C127, and C131 to C133, or a pharmaceutically acceptable salt thereof, is wherein R^{aa}, R^{bb}, R^{cc}, and R^{dd} are independently selected from hydrogen and methyl.
C135. In embodiment C135, the compound of any one of embodiments C1 to C117, C119 to C127, and C131 to C133, or a pharmaceutically acceptable salt thereof, is wherein R^{aa}, R^{bb}, R^{cc} and R^{dd} are independently selected from hydrogen and methoxy.
C136. In embodiment C136, the compound of any one of embodiments C1 to C117, C119 to C127, and C131 to C133, or a pharmaceutically acceptable salt thereof, is wherein R^{aa}, R^{bb}, Roc and R^{dd} are independently selected from hydrogen and fluoro.
C137. In embodiment C137, the compound of any one of embodiments C1 to C117, C119 to C127, and C131 to C133, or a pharmaceutically acceptable salt thereof, is wherein R^{aa}, R^{bb}, R^{cc}, and R^{dd} are independently selected from hydrogen, trifluoromethyl, and difluoromethyl.
C138. In embodiment C138, the compound of any one of embodiments C1 to C117, C119 to C127, C131, and C133, or a pharmaceutically acceptable salt thereof, is wherein R^{aa}, R^{bb}, R^{cc}, and R^{dd} are independently selected from hydrogen and trifluoromethoxy.
C139. In embodiment C139, the compound of any one of embodiments C1 to C117, C119 to C127, and C131 to C133, or a pharmaceutically acceptable salt thereof, is wherein R^{aa}, R^{bb}, Roc and R^{dd} are independently selected from hydrogen, fluoro, and trifluoromethyl.
C140. In embodiment C140, the compound of any one of embodiments C1 to C108, or a pharmaceutically acceptable salt thereof, is wherein the Degron is an E3 ubiquitin ligase ligand of formula (ii):

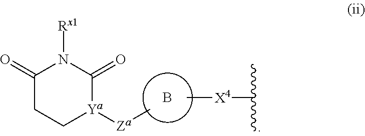

C141. In embodiment C141, the compound of any one of embodiments C1 to C108, and C140, or a pharmaceutically acceptable salt thereof, is wherein Y^{a} is CH.
C142. In embodiment C142, the compound of any one of embodiments C1 to C108, and C140, or a pharmaceutically acceptable salt thereof, is wherein Y^{a} is N.
C143. In embodiment C143, the compound of any one of embodiments C1 to C108, and C140 to C142, or a pharmaceutically acceptable salt thereof, is wherein Z^{a} is a bond, —NH—, —O—, or —NHC(O)—.
C144. In embodiment C144, the compound of any one of embodiments C1 to C108, and C140 to C143, or a pharmaceutically acceptable salt thereof, is wherein Z^{a} is a bond, —NH—, or —NHC(O)—.
C145. In embodiment C145, the compound of any one of embodiments C1 to C108, and C140 to C144, or a pharmaceutically acceptable salt thereof, is wherein Z^{a} is a bond.
C146. In embodiment C146, the compound of any one of embodiments C1 to C108, and C140 to C144, or a pharmaceutically acceptable salt thereof, is wherein Z^{a} is —NH—, or —NHC(O)—.
C147. In embodiment C147, the compound of any one of embodiments C1 to C108, C140 to C144, and C146, or a pharmaceutically acceptable salt thereof, is wherein Z^{a} is —NH—.
C148. In embodiment C148, the compound of any one of embodiments C1 to C108, C140 to C144, and C146, or a pharmaceutically acceptable salt thereof, is wherein Z^{a} is —NHC(O)—.
C149. In embodiment C149, the compound of any one of embodiments C1 to C108 and C140 to C148, or a pharmaceutically acceptable salt thereof, is wherein ring B is phenylene substituted with R^{ee} and R^{ff}.
C150. In embodiment C150, the compound of any one of embodiments C1 to C108 and C140 to C148, or a pharmaceutically acceptable salt thereof, is wherein ring B is cyclylaminylene substituted with R^{ee} and R^{ff}.
C151. In embodiment C151, the compound of any one of embodiments C1 to C108 and C140 to C148, or a pharmaceutically acceptable salt thereof, is wherein ring B is 5- or 6-membered monocyclic heteroarylene or a 9- or 10-membered fused bicyclic heteroarylene, wherein each heteroarylene ring contains one to three nitrogen ring atoms and each ring is substituted with R^{ee} and R^{ff}.
C152. In embodiment C152, the compound of any one of embodiments C1 to C108 C140 to C148, and C151, or a pharmaceutically acceptable salt thereof, is wherein ring B is 5- or 6-membered monocyclic heteroarylene containing one or two nitrogen ring atoms substituted with R^{ee} and R^{ff}.
C153. In embodiment C153, the compound of any one of embodiments C1 to C108 C140 to C148, and C151, or a pharmaceutically acceptable salt thereof, is wherein ring B is a 9- or 10-membered fused bicyclic heteroarylene containing one to three nitrogen ring atoms (and not containing any additional heteroatoms) and substituted with R^{ee} and R^{ff}.
C154. In embodiment C154, the compound of any one of embodiments C1 to C108, C140 to C148, C151, and C153, or a pharmaceutically acceptable salt thereof, is wherein ring B is a 9- or 10-membered fused bicyclic heteroarylene containing one or two nitrogen ring atoms and substituted with R^{ee} and R^{ff}.
C155. In embodiment C155, the compound of any one of embodiments C1 to C108 and C140 to C154, or a pharmaceutically acceptable salt thereof, is wherein the E3 ubiquitin ligase ligand of formula (ii) is:

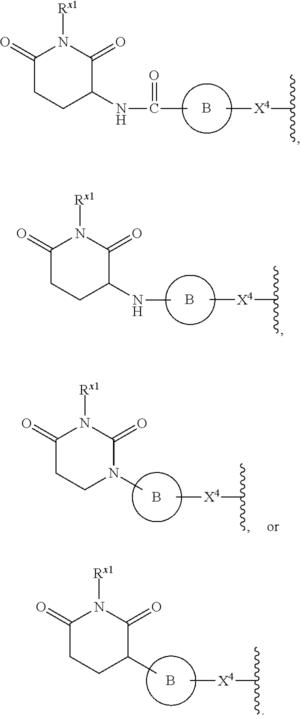

C156. In embodiment C156, the compound of any one of embodiments C1 to C108 and C140 to C155, or a pharmaceutically acceptable salt thereof, is wherein the E3 ubiquitin ligase ligand of formula (ii) is:

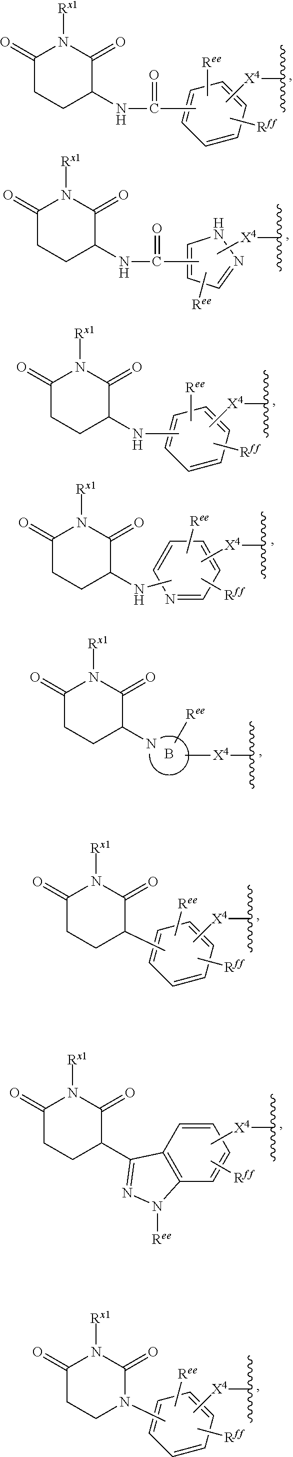

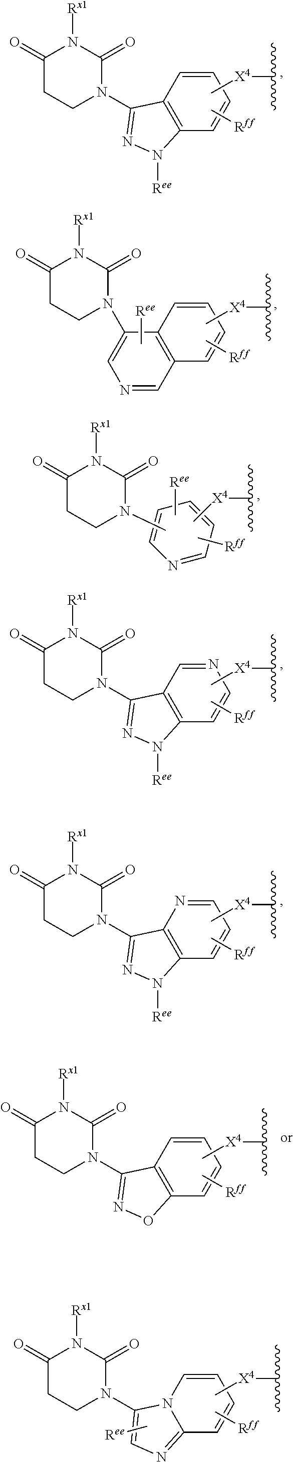

where ring B is cyclylaminylene.
C157. In embodiment C157, the compound of any one of embodiments C1 to C108 and C140 to C156, or a pharmaceutically acceptable salt thereof, is wherein the E3 ubiquitin ligase ligand of formula (ii) is:

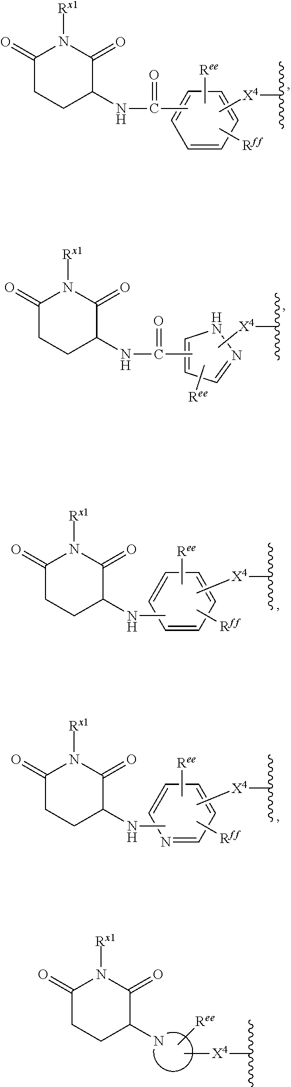

where ring B is cyclylaminylene,

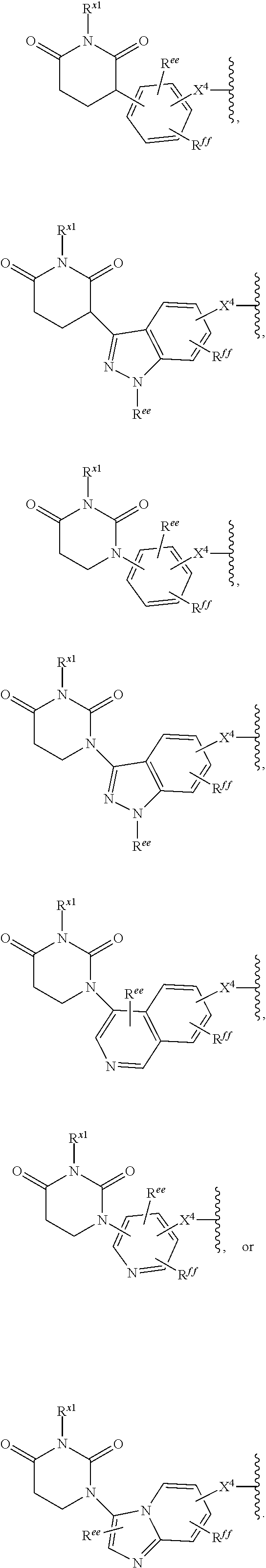

C158. In embodiment C158, the compound of any one of embodiments C1 to C108 and C140 to C157, or a pharmaceutically acceptable salt thereof, is wherein the E3 ubiquitin ligase ligand of formula (ii) is

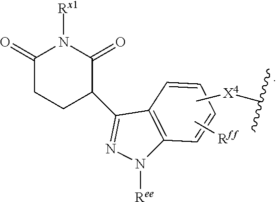

C159. In embodiment C159, the compound of any one of embodiments C1 to C108 and C140 to C157, or a pharmaceutically acceptable salt thereof, is wherein the E3 ubiquitin ligase ligand of formula (ii) is:

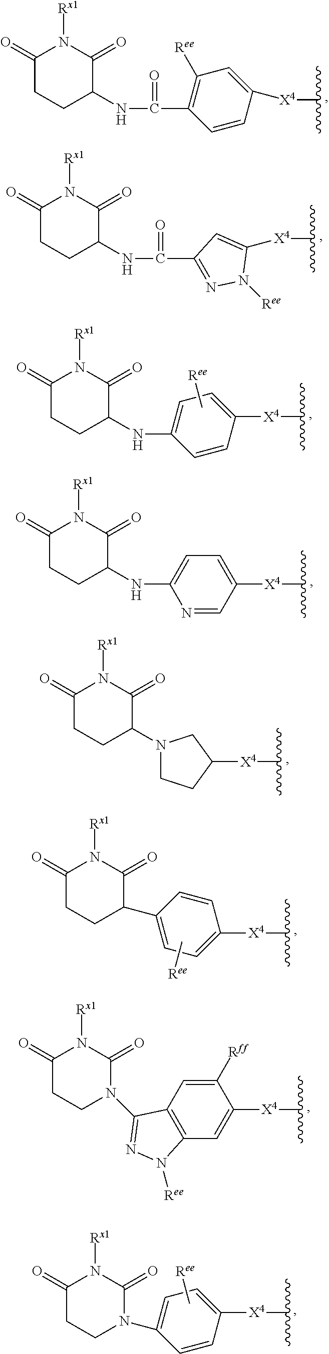

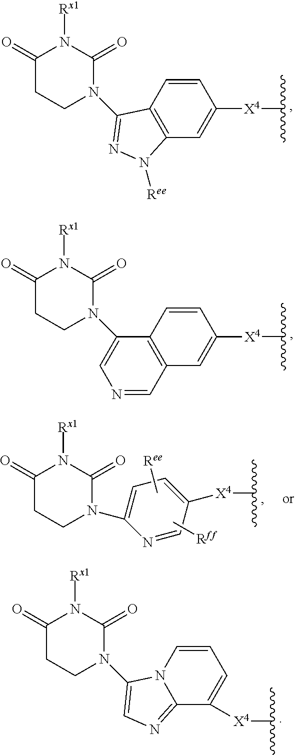

C160. In embodiment C160, the compound of any one of embodiments C1 to C108, C140 to C157, and C159, or a pharmaceutically acceptable salt thereof, is wherein the E3 ubiquitin ligase ligand of formula (ii) is

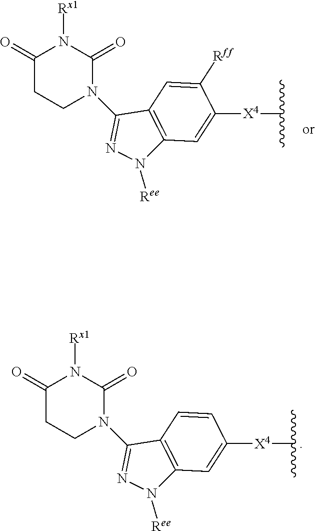

C161. In embodiment C161, the compound of any one of embodiments C1 to C108 and C140 to C160, or a pharmaceutically acceptable salt thereof, is wherein each R^{ee} and R^{ff} are independently selected from hydrogen, alkyl, alkoxy, halo, cyano, haloalkyl, and haloalkoxy.
C162. In embodiment C162, the compound of any one of embodiments C1 to C108 and C140 to C160, or a pharmaceutically acceptable salt thereof, is wherein R^{ee} and R^{ff} are independently selected from hydrogen, alkyl, cycloalkyl, alkoxy, halo, haloalkyl, and cyano.
C163. In embodiment C163, the compound of any one of embodiments C1 to C108 and C140 to C162, or a pharmaceutically acceptable salt thereof, is wherein R^{ee} and R^{ff} are independently selected from hydrogen, methyl, ethyl, isopropyl, cyclopropyl, methoxy, ethoxy, fluoro, chloro, trifluoromethyl, 2,2,2-trifluoroethyl, difluoromethyl, difluoromethoxy, trifluoromethoxy, and cyano.
C164. In embodiment C164, the compound of any one of embodiments C1 to C108 and C140 to C163, or a pharmaceutically acceptable salt thereof, is wherein R^{ee} and R^{ff} are independently selected from hydrogen, methyl, ethyl, and isopropyl.
C165. In embodiment C165, the compound of any one of embodiments C1 to C108 and C140 to C163, or a pharmaceutically acceptable salt thereof, is wherein R^{ee} and R^{ff} are independently selected from hydrogen and methoxy.
C166. In embodiment C166, the compound of any one of embodiments C1 to C108 and C140 to C163, or a pharmaceutically acceptable salt thereof, is wherein R^{ee} and R^{ff} are independently selected from hydrogen, methyl, ethyl, isopropyl, chloro, and fluoro.
C167. In embodiment C167, the compound of any one of embodiments C1 to C108 and C140 to C163, or a pharmaceutically acceptable salt thereof, is wherein one of R^{ee} and R^{ff} is hydrogen or fluoro and the other of R^{ee} and R^{ff} is selected from hydrogen, trifluoromethyl, 2,2,2-trifluoroethyl, and difluoromethyl.
C168. In embodiment C168, the compound of any one of embodiments C1 to C108, C140 to C161, and C163, or a pharmaceutically acceptable salt thereof, is wherein R^{ee} and R^{ff} are independently selected from hydrogen, difluoromethoxy, and trifluoromethoxy.
C169. In embodiment C169, the compound of any one of embodiments C1 to C108 and C140 to C163, or a pharmaceutically acceptable salt thereof, is wherein R^{ee} and R^{ff} are independently selected from hydrogen, chloro, fluoro, and trifluoromethyl.
C170. In embodiment C170, the compound of any one of embodiments C1 to C108 and C140 to C163, or a pharmaceutically acceptable salt thereof, is wherein R^{ee} and R^{ff} are hydrogen.
C171. In embodiment C171, the compound of any one of embodiments C1 to C108 and C140 to C163, or a pharmaceutically acceptable salt thereof, is wherein R^{ee} and R^{ff} are chloro.
C172. In embodiment C172, the compound of any one of embodiments C1 to C108 and C140 to C163, or a pharmaceutically acceptable salt thereof, is wherein R^{ee} and R^{ff} are fluoro.
C173. In embodiment C173, the compound of any one of embodiments C1 to C108 and C140 to C163, or a pharmaceutically acceptable salt thereof, is wherein R^{ee} and R^{ff} are independently trifluoromethyl or 2,2,2-trifluoroethyl.
C174. In embodiment C174, the compound of any one of embodiments C1 to C107, or a pharmaceutically acceptable salt thereof, is wherein the Degron is an E3 ubiquitin ligase ligand of formula (iii), (iv), (v), or (vi).
C175. In embodiment C175, the compound of any one of embodiments C1 to C107 and C174, or a pharmaceutically acceptable salt thereof, is wherein the Degron is an E3 ubiquitin ligase ligand of formula (iv) or (v).
C176. In embodiment C176, the compound of any one of embodiments C1 to C107, C174, and C175, or a pharmaceutically acceptable salt thereof, is wherein R^{y}, R^{y1}, and R^{y2} are 1-fluorocycloprop-1-yl and W^{a} is bond, S, or methylene.
C177. In embodiment C177, the compound of any one of embodiments C1 to C107 and C174 to C176, or a pharmaceutically acceptable salt thereof, is wherein W^{a} is S.
C178. In embodiment C178, the compound of any one of embodiments C1 to C173, or a pharmaceutically acceptable salt thereof, is wherein X¹, X², X³, and X⁴ are each a bond.
C179. In embodiment C179, the compound of any one of embodiments C1 to C173, or a pharmaceutically acceptable salt thereof, is wherein X¹, X², X³, and X⁴ are each independently selected from alkylene. In a subembodiment of C179, each alkylene is methylene.
C180. In embodiment C180, the compound of any one of embodiments C1 to C173, or a pharmaceutically acceptable salt thereof, is wherein X¹, X², X³, and X⁴ are each —O—.
C181. In embodiment C181, the compound of any one of embodiments C1 to C173, or a pharmaceutically acceptable salt thereof, is wherein X¹, X², X³, and X⁴ are each independently selected from —(O-alkylene)-.
C181a. In embodiment C181a, the compound of any one of embodiments C1 to C173, or a pharmaceutically acceptable salt thereof, is wherein X¹, X², X³, and X⁴ are each independently selected from -(alkylene-O)—.
C182. In embodiment C182, the compound of any one of embodiments C1 to C173, or a pharmaceutically acceptable salt thereof, is wherein X¹, X², X³, and X⁴ are each independently selected from —(NR^{gg}-alkylene)-.
C183. In embodiment C183, the compound of any one of embodiments C1 to C173, or a pharmaceutically acceptable salt thereof, is wherein X¹, X², X³, and X⁴ are each independently selected from -(alkylene-NR^{hh})—.
C184. In embodiment C184, the compound of any one of embodiments C1 to C173, or a pharmaceutically acceptable salt thereof, is wherein X¹, X², X³, and X⁴ are each

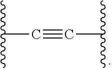 [SMILES: *C#C*]

C185. In embodiment C185, the compound of any one of embodiments C1 to C173, or a pharmaceutically acceptable salt thereof, is wherein X¹, X², X³ and X⁴ are each —NH—.
C186. In embodiment C186, the compound of any one of embodiments C1 to C173, or a pharmaceutically acceptable salt thereof, is wherein X¹, X², X³, and X⁴ are each independently selected from —N(alkyl)-. In a subembodiment of C186, each —N(alkyl)- is independently —N(methyl)- or —N(ethyl)-.
C187. In embodiment C187, the compound of any one of embodiments C1 to C173, or a pharmaceutically acceptable salt thereof, is wherein X¹, X², X³, and X⁴ are each —C(═O)—.
C188. In embodiment C188, the compound of any one of embodiments C1 to C173, or a pharmaceutically acceptable salt thereof, is wherein X¹, X², X³, and X⁴ are each independently —NR^{jj}C(═O)—.
C189. In embodiment C189, the compound of any one of embodiments C1 to C173, or a pharmaceutically acceptable salt thereof, is wherein X¹, X², X³, and X⁴ are each independently —C(═O)NR^{kk}—.
C190. In embodiment C190, the compound of any one of embodiments C1 to C173, C182, C183, C188, and C189, or a pharmaceutically acceptable salt thereof, is wherein R^{gg}, R^{hh}, R^{jj}, and R^{kk} are each independently hydrogen or alkyl.
C190a. In embodiment C190a, the compound of any one of embodiments C1 to C190 is wherein at least two of —Z¹—Z²—Z³—Z⁴—Z⁵—Z⁶— are not a bond. In a sub-embodiment of C190a, the compound is wherein at least three of —Z¹—Z²—Z³—Z⁴—Z⁵—Z⁶— are not a bond. In a sub-embodiment of
C190a, the compound is wherein at least four of —Z¹—Z²—Z³—Z⁴—Z⁵—Z⁶— are not a bond.
C191. In embodiment C191, the compound of any one of embodiments C1 to C190a, or a pharmaceutically acceptable salt thereof, is wherein Z⁶ is —S(O)₂—.
C192. In embodiment C192, the compound of any one of embodiments C1 to C191, or a pharmaceutically acceptable salt thereof, is wherein Z⁵ is phenylene, monocyclic heteroarylene, or heterocycylene, where each ring is substituted with R^{q} and R^{r}.
C193. In embodiment C193, the compound of any one of embodiments C1 to C192, or a pharmaceutically acceptable salt thereof, is wherein Z⁵ is phenylene, monocyclic heteroarylene, or heterocycylene, where each ring is substituted with R^{q} and R^{r} and one and only one of Z¹ and X¹ is a bond, one and only one of Z¹ and X² is a bond, one and only one of Z¹ and X³, and one and only one of Z¹ and X⁴ is a bond (for sake of clarity, when X¹, X², X³, and X⁴ are not a bond, then X¹, X², X³, and X⁴ are as described in any one of embodiments C1 and C179 to C189).
C194. In embodiment C194, the compound of any one of embodiments C1 to C177, or a pharmaceutically acceptable salt thereof, is wherein:
X¹, X², X³, and X⁴ are independently a bond, —(O-alkylene)-, —(NR^{gg}-alkylene)-,

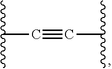 [SMILES: *C#C*]

—NH—, or —N(alkyl)-, where R^{gg} is hydrogen or alkyl and each alkylene is independently optionally substituted with one or two fluoro (or X¹, X², X³, and X⁴ bond when E3 ubiquitin ligase ligand is (iii) to (vi));
Z¹ is a bond, alkylene, —(CO)NR—, —(O-alkylene)ₐ-, -(alkylene-O)ₐ—, phenylene, or heterocyclylene, where each ring is substituted with R^{h} and Rⁱ;
Z² is a bond, alkylene, —(O-alkylene)_{b}-, -(alkylene-O)_{b}—, cycloalkylene, or heterocyclylene, where each ring is substituted with R^{j} and R^{k};
Z³ is a bond, alkylene, —C(O)NR—, —NR′(CO)—, —O—, —NR″—, cycloalkylene, phenylene, -(alkylene)-phenylene-, -phenylene-(alkylene)-, monocyclic heteroarylene, -(alkylene)-monocyclic heteroarylene-, -monocyclic heteroarylene-(alkylene)-, heterocyclylene, -(alkylene)-heterocyclylene-, -heterocyclylene-(alkylene)-, bridged heterocyclylene, -(alkylene)-bridged heterocyclylene-, -bridged heterocyclylene-(alkylene)-, spiro heterocyclylene, -(alkylene)-spiro heterocyclylene-, -spiro heterocyclylene-(alkylene)-, or monocyclic heteroarylene, where each ring, by itself or as part of another group, is substituted with R^{m} and Rⁿ;
Z⁴ is a bond, -(alkylene-NR″)—, —(NR″-alkylene)-, —O—, —NR″—, cycloalkylene, phenylene, monocyclic heteroarylene, heterocyclylene, -(alkylene)-heterocyclylene-, -heterocyclylene-(alkylene)-, fused heterocyclylene, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p};
Z⁵ is a bond; and
Z⁶ is —S(O)₂—; and
wherein each alkylene in Z¹, Z², Z³, and Z⁴, by itself or as part of another group, is independently substituted with R^{s} and R^{t}.
C195. In embodiment C195, the compound of any one of embodiments C1 to C177, or a pharmaceutically acceptable salt thereof, is wherein:
X¹, X², X³, X⁴, and Z¹ are each a bond;
Z² is a bond, alkylene, cycloalkylene, or heterocyclylene, where each ring is substituted with R^{j} and R^{k};
Z³ is a bond, alkylene, —C(O)NR—, —NR′(CO)—, —O—, —NR″—, cycloalkylene, phenylene, monocyclic heteroarylene, heterocyclylene, bicyclic heterocyclylene, bridged heterocyclylene, fused heterocyclylene, or spiro heterocyclylene, where each ring is substituted with R^{m} and Rⁿ;
Z⁴ is a bond, alkylene, —O—, cycloalkylene, phenylene, monocyclic heteroarylene, heterocyclylene, fused heterocyclylene, or spiro heterocyclylene, where each ring is substituted with R^{o} and R^{p};
Z⁵ is phenylene, monocyclic heteroarylene, or heterocycylene, where each ring is substituted with R^{q} and R^{r}; and
Z⁶ is —S(O)₂—; and
wherein each alkylene in Z², Z³, and Z⁴ is independently substituted with R^{s} and R^{t}.
C196. In embodiment C196, the compound of any one of embodiments C1 to C177 and C195, or a pharmaceutically acceptable salt thereof, is wherein:
X¹, X², X³, X⁴, Z¹, and Z² are each a bond;
Z³ is cycloalkylene, phenylene, monocyclic heteroarylene, heterocyclylene, bicyclic heterocyclylene, bridged heterocyclylene, fused heterocyclylene, or spiro heterocyclylene, where each ring is substituted with R^{m} and Rⁿ;
Z⁴ is a bond, alkylene, —O—, cycloalkylene, phenylene, monocyclic heteroarylene, heterocyclylene, fused heterocyclylene, or spiro heterocyclylene, where each ring is substituted with R^{o} and R^{p} independently selected from hydrogen, deuterium, alkyl, alkoxy, hydroxy, cyano, halo, haloalkyl, and haloalkoxy;
Z⁵ is phenylene, monocyclic heteroarylene, or heterocycylene, where each ring is substituted with R^{q} and R^{r}; and
Z⁶ is —S(O)₂—; and
wherein alkylene in Z⁴ is substituted with R^{s} and R^{t}.
C197. In embodiment C197, the compound of any one of embodiments C1 to C177, C195, and C196, or a pharmaceutically acceptable salt thereof, is wherein:
X¹, X², X³, X⁴, Z¹, and Z² are each a bond;
Z³ is heterocyclylene, bicyclic heterocyclylene, bridged heterocyclylene, fused heterocyclylene, or spiro heterocyclylene, where each ring is substituted with R^{m} and Rⁿ independently selected from hydrogen, deuterium, alkyl, alkoxy, hydroxy, cyano, halo, haloalkyl, and haloalkoxy;
Z⁴ is alkylene, —O—, cycloalkylene, monocyclic heteroarylene, heterocyclylene, fused heterocyclylene, or spiro heterocyclylene, where each ring is substituted with R^{o} and R^{p} independently selected from hydrogen, deuterium, alkyl, alkoxy, hydroxy, cyano, halo, haloalkyl, and haloalkoxy;
Z⁵ is phenylene, monocyclic heteroarylene, or heterocycylene, where each ring is substituted with R^{q} and R^{r} independently selected from hydrogen, deuterium, alkyl, alkoxy, hydroxy, cyano, halo, haloalkyl, and haloalkoxy; and
Z⁶ is —S(O)₂—; and
wherein alkylene in Z⁴ is substituted with substituted with R^{s} and R^{t}.
C198. In embodiment C198, the compound of any one of embodiments C1 to C177 and C195 to C197, or a pharmaceutically acceptable salt thereof, is wherein:
X¹, X², X³, and X⁴, Z¹, and Z² are each a bond;
Z³ is heterocyclylene, bridged heterocyclylene, or spiro heterocyclylene, where each ring is substituted with R^{m} and Rⁿ independently selected from hydrogen, deuterium, alkyl, alkoxy, hydroxy, cyano, halo, haloalkyl, and haloalkoxy;
Z⁴ is alkylene, —O—, cycloalkylene, or heterocyclylene, where each ring is substituted with R^{o} and R^{p} independently selected from hydrogen, deuterium, alkyl, alkoxy, halo, haloalkyl, haloalkoxy, cyano, and hydroxy, preferably hydrogen, deuterium, alkyl, alkoxy, hydroxy, cyano, halo, haloalkyl, and haloalkoxy;
Z⁵ is phenylene, monocyclic heteroarylene, or heterocycylene, where each ring is substituted with R^{q} and R^{r} independently selected from hydrogen, deuterium, alkyl, alkoxy, hydroxy, cyano, halo, haloalkyl, and haloalkoxy; and
Z⁶ is —S(O)₂—; and
wherein alkylene in Z⁴ is substituted with R^{s} and R^{t}.
C199. In embodiment C119, the compound of any one of embodiments C1 to C177 and C195 to C198, or a pharmaceutically acceptable salt thereof, is wherein:
X¹, X², X³, X⁴, Z¹, and Z² are each a bond;
Z³ is heterocyclylene, bridged heterocyclylene, or spiro heterocyclylene, where each ring is substituted with R^{m} and Rⁿ independently selected from hydrogen, deuterium, alkyl, alkoxy, hydroxy, cyano, halo, haloalkyl, and haloalkoxy;
Z⁴ is alkylene, —O—, cycloalkylene, or heterocyclylene, where each ring is substituted with R^{o} and R^{p} independently selected from hydrogen, deuterium, alkyl, alkoxy, halo, haloalkyl, haloalkoxy, cyano, and hydroxy, preferably hydrogen, deuterium, alkyl, alkoxy, halo, haloalkyl, and haloalkoxy, preferably Z⁴ is alkylene or —O—;
Z⁵ is phenylene or monocyclic heteroarylene, each ring substituted with R^{a} and R^{r} independently selected from hydrogen, deuterium, alkyl, alkoxy, hydroxy, cyano, halo, haloalkyl, and haloalkoxy; and
Z⁶ is —S(O)₂—; and
wherein alkylene in Z⁴ is substituted with R^{s} and R^{t}.
C200. In embodiment C200, the compound of any one of embodiments C1 to C177 and C195, or a pharmaceutically acceptable salt thereof, is wherein:
X¹, X², X³, X⁴, and Z¹ are each a bond;
Z² is cycloalkylene or heterocyclylene, where each ring is substituted with R^{j} and R^{k} independently selected from hydrogen, deuterium, alkyl, alkoxy, halo, haloalkyl, and haloalkoxy;
Z³ is cycloalkylene, phenylene, monocyclic heteroarylene, heterocyclylene, bicyclic heterocyclylene, bridged heterocyclylene, fused heterocyclylene, or spiro heterocyclylene, where each ring is substituted with R^{m} and Rⁿ independently selected from hydrogen, deuterium, alkyl, alkoxy, hydroxy, cyano, halo, haloalkyl, and haloalkoxy;
Z⁴ is a bond, alkylene, or —O—;
Z⁵ is phenylene, monocyclic heteroarylene (e.g., pyridindiyl), or heterocycylene, where each ring is substituted with R^{q} and R^{r} independently selected from hydrogen, deuterium, alkyl, alkoxy, hydroxy, cyano, halo, haloalkyl, and haloalkoxy; and
Z⁶ is —S(O)₂—; and
wherein alkylene in Z⁴ is substituted with R^{s} and R^{t}.
C201. In embodiment C201, the compound of any one of embodiments C1 to C177, C191, and C195, or a pharmaceutically acceptable salt thereof, is wherein:
X¹, X², X³, X⁴, and Z¹ are each a bond;
Z² is heterocyclylene substituted with R^{j} and R^{k} independently selected from hydrogen, deuterium, alkyl, alkoxy, halo, haloalkyl, and haloalkoxy;
Z³ is heterocyclylene substituted with R^{m} and Rⁿ independently selected from hydrogen, deuterium, alkyl, alkoxy, hydroxy, cyano, halo, haloalkyl, and haloalkoxy;
Z⁴ is a bond, alkylene, or —O—;
Z⁵ is phenylene or monocyclic heteroarylene, each ring substituted with R^{q} and R^{r} independently selected from hydrogen, deuterium, alkyl, alkoxy, hydroxy, cyano, halo, haloalkyl, and haloalkoxy; and
Z⁶ is —S(O)₂—; and
wherein alkylene in Z⁴ is substituted with R^{s} and R^{t}.
C202. In embodiment C202, the compound of any one of embodiments C1 to C177, C178, C191, and C192, or a pharmaceutically acceptable salt thereof, is wherein:
X¹, X², X³, X⁴, and Z¹ are each a bond;
Z² is heterocyclylene substituted with R^{j} and R^{k}, preferably R^{j} and R^{k} are independently selected from hydrogen, deuterium, alkyl, alkoxy, halo, haloalkyl, and haloalkoxy;
Z³ is a bond, alkylene, or —O—;
Z⁴ is heterocyclylene, bridged heterocyclylene, or spiro heterocyclylene, where each ring is substituted with R^{o} and R^{p}, preferably R^{o} and R^{p} are independently selected from hydrogen, deuterium, alkyl, alkoxy, hydroxy, cyano, halo, haloalkyl, and haloalkoxy;
Z⁵ is phenylene or monocyclic heteroarylene, each ring substituted with R^{q} and R^{r}, preferably R^{q} and R^{r} are independently selected from hydrogen, deuterium, alkyl, alkoxy, hydroxy, cyano, halo, haloalkyl, and haloalkoxy; and 30
Z⁶ is —S(O)₂—; and
wherein alkylene in Z³ is substituted with R^{s} and R^{t}.
C203. In embodiment C203, the compound of any one of embodiments C1 to C177, or a pharmaceutically acceptable salt thereof, is wherein Z⁴ is heterocyclylene or spiro heterocyclylene, where each ring is substituted with R^{o} and R^{p}, preferably R^{o} and R^{p} are independently selected from hydrogen, deuterium, alkyl, alkoxy, hydroxy, cyano, halo, haloalkyl, and haloalkoxy.
C204. In embodiment C204, the compound of any one of embodiments C1 to C177, C195, and C196 or a pharmaceutically acceptable salt thereof, is wherein:
X¹, X², X³, X⁴, Z¹ and Z² are each a bond;
Z³ is heterocyclylene, where each ring is substituted with R^{m} and Rⁿ independently selected from hydrogen, deuterium, alkyl, alkoxy, hydroxy, cyano, halo, haloalkyl, and haloalkoxy;
Z⁴ is cycloalkylene substituted with R^{o} and R^{p} independently selected from hydrogen, alkyl, alkoxy, halo, haloalkyl, and haloalkoxy;
Z⁵ is phenylene or monocyclic heteroarylene, each ring substituted with R^{q} and R^{r} independently selected from hydrogen, deuterium, alkyl, alkoxy, hydroxy, cyano, halo, haloalkyl, and haloalkoxy; and
Z⁶ is —S(O)₂—.
C205. In embodiment C205, the compound of any one of embodiments C1 to C177, or a pharmaceutically acceptable salt thereof, is wherein one and only one of X¹ and Z¹, or one and only one of X² and Z¹, or one and only one of X³ and Z¹, or one and only one of X⁴ and Z¹ is a bond.
C206. In embodiment C206, the compound of any one of embodiments C1 to C177, or a pharmaceutically acceptable salt thereof, is wherein X¹, X², X³, X⁴, and Z¹ are each a bond.
C207. In embodiment C207, the compound of any one of embodiments C1 to C177, C205, and C206, or a pharmaceutically acceptable salt thereof, is wherein Z² is heterocyclylene or bridged heterocyclylene, each ring substituted with R^{j} and R^{k}
C208. In embodiment C208, the compound of any one of embodiments C1 to C177, C205, and C206, or a pharmaceutically acceptable salt thereof, is wherein Z² is a bond.
C209. In embodiment C209, the compound of any one of embodiments C1 to C177, and C205 to C208, or a pharmaceutically acceptable salt thereof, is wherein:
Z³ is alkylene, cycloalkylene, phenylene, -(alkylene)-phenylene-, -phenylene-(alkylene)-, monocyclic heteroarylene, -(alkylene)-monocyclic heteroarylene-, -monocyclic heteroarylene-(alkylene)-, heterocyclylene, -(alkylene)-heterocyclylene-, -heterocyclylene-(alkylene)-, bicyclic heterocyclylene, bridged heterocyclylene, -(alkylene)-bridged heterocyclylene-, -bridged heterocyclylene-(alkylene)-, fused heterocyclylene, spiro heterocyclylene, -(alkylene)-spiro heterocyclylene-, or -spiro heterocyclylene-(alkylene), where each ring, by itself or as part of another group, is substituted with R^{m} and Rⁿ;
Z⁴ is alkylene, -(alkylene-NR″)—, —(NR″-alkylene)-, —O—, —NR″—, —(O-alkylene)_{d}-, -(alkylene-O)—, cycloalkylene, -(alkylene)-cycloalkylene-, -cycloalkylene-(alkylene)-, spiro cyclolalkylene, phenylene, heteroarylene, heterocyclylene, -(alkylene)-heterocyclylene-, -heterocyclylene-(alkylene)-, fused heterocyclylene, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p};
Z⁵ is a bond, -alkylene, —NR″—, —O—, —C(O)—, —S(O)₂—, —NR′(CO)—, —C(O)NR—, phenylene, monocyclic heteroarylene, or heterocycylene, where each ring is substituted with R^{q} and R^{r}; and
Z⁶ is a bond, alkylene, —NR″—, —O—, -(alkylene-O)—, —C(O)—, —S(O)₂—, —NR′(CO)—, or —C(O)NR—; and
and each alkylene in Z³, Z⁴, Z⁵, and Z⁶, itself or as part of another group, is independently substituted with R^{s} and R^{t}.
C210. In embodiment C210, the compound of any one of embodiments C1 to C177, and C205 to C209, or a pharmaceutically acceptable salt thereof, is wherein:
Z³ is alkylene, cycloalkylene, phenylene, -(alkylene)-phenylene-, -phenylene-(alkylene)-, monocyclic heteroarylene, -(alkylene)-monocyclic heteroarylene-, -monocyclic heteroarylene-(alkylene)-, heterocyclylene, -(alkylene)-heterocyclylene-, -heterocyclylene-(alkylene)-, bicyclic heterocyclylene, bridged heterocyclylene, -(alkylene)-bridged heterocyclylene-, -bridged heterocyclylene-(alkylene)-, fused heterocyclylene, spiro heterocyclylene, -(alkylene)-spiro heterocyclylene-, or -spiro heterocyclylene-(alkylene), where each ring, by itself or as part of another group, is substituted with R^{m} and Rⁿ;
Z⁴ is alkylene, -(alkylene-NR″)—, —(NR″-alkylene)-, —O—, —NR″—, —(O-alkylene)_{d}-, -(alkylene-O)_{d}—, cycloalkylene, -(alkylene)-cycloalkylene-, -cycloalkylene-(alkylene)-, spiro cyclolalkylene, phenylene, heteroarylene, heterocyclylene, -(alkylene)-heterocyclylene-, -heterocyclylene-(alkylene)-, fused heterocyclylene, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p};
Z⁵ is a bond, -alkylene, —NR″—, —O—, —C(O)—, —S(O)₂—, —NR′(CO)—, —C(O)NR—, phenylene, monocyclic heteroarylene, or heterocycylene, where each ring is substituted with R^{q} and R^{r}; and
Z⁶ is —S(O)₂—; and
and each alkylene in Z³, Z⁴, and Z⁵, itself or as part of another group, is independently substituted with R^{s} and R^{t}.
C211. In embodiment C211, the compound of any one of embodiments C1 to C178, C191 to C193, C206, and C208 to C210, or a pharmaceutically acceptable salt thereof, is wherein:
X¹, X², X³, X⁴, Z¹, and Z² are each a bond;
Z³ is-heterocyclylene-(alkylene)-, where heterocyclylene is substituted with R^{m} and Rⁿ and alkylene is substituted with R^{s} and R^{t};
Z⁴ is phenylene or monocyclic heteroarylene, where each ring is substituted with R^{o} and R^{p};
Z⁵ is phenylene substituted with R^{q} and R^{r}; and
Z⁶ is —S(O)₂—.
C212. In embodiment C212, the compound of any one of embodiments C1 to C177, and C205 to C210, or a pharmaceutically acceptable salt thereof, is wherein:
Z³ is alkylene, phenylene, -(alkylene)-phenylene-, -phenylene-(alkylene)-, monocyclic heteroarylene, -(alkylene)-monocyclic heteroarylene-, -monocyclic heteroarylene-(alkylene)-, heterocyclylene, -(alkylene)-heterocyclylene-, -heterocyclylene-(alkylene)-, bridged heterocyclylene, -(alkylene)-bridged heterocyclylene-, -bridged heterocyclylene-(alkylene)-, spiro heterocyclylene, -(alkylene)-spiro heterocyclylene-, or -spiro heterocyclylene-(alkylene), where each ring, by itself or as part of another group, is substituted with R^{o} and R^{p};
Z⁴ is alkylene, -(alkylene-NR″)—, —(NR″-alkylene)-, —O—, —NR″—, —(O-alkylene)_{d}-, -(alkylene-O)_{d}—, cycloalkylene, -(alkylene)-cycloalkylene-, -cycloalkylene-(alkylene)-, spiro cyclolalkylene, phenylene, heteroarylene, heterocyclylene, -(alkylene)-heterocyclylene-, -heterocyclylene-(alkylene)-, fused heterocyclylene, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p};
Z⁵ is phenylene, monocyclic heteroarylene, or heterocycylene, where each ring is substituted with R^{q} and R^{r}; and
Z⁶ is —S(O)₂—; and
and each alkylene in Z³ and Z⁴, itself or as part of another group, is independently substituted with R^{s} and R^{t}.
C212a. In embodiment C212a, the compound of any one of embodiments C1 to C177, and C205 to C210, or a pharmaceutically acceptable salt thereof, is wherein:
Z³ is alkylene, phenylene, -(alkylene)-phenylene-, -phenylene-(alkylene)-, monocyclic heteroarylene, -(alkylene)-monocyclic heteroarylene-, -monocyclic heteroarylene-(alkylene)-, where each ring, by itself or as part of another group, is substituted with R^{m} and Rⁿ; Z⁴ is alkylene, -(alkylene-NR″)—, —(NR″-alkylene)-, —O—, —NR″—, —(O-alkylene)_{d}-, -(alkylene-O)_{d}—, cycloalkylene, -(alkylene)-cycloalkylene-, -cycloalkylene-(alkylene)-, spiro cyclolalkylene, phenylene, heteroarylene, heterocyclylene, -(alkylene)-heterocyclylene-, -heterocyclylene-(alkylene)-, fused heterocyclylene, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p};
Z⁵ is phenylene, monocyclic heteroarylene, or heterocycylene, where each ring is substituted with R^{s} and R^{t}; and
Z⁶ is —S(O)₂; and
and each alkylene in Z³ and Z⁴, itself or as part of another group, is independently substituted with R^{s} and R^{t}.
C213. In embodiment C213, the compound of any one of embodiments C1 to C177, and C205 to C212, or a pharmaceutically acceptable salt thereof, is wherein:
Z³ is heterocyclylene, -(alkylene)-heterocyclylene-, -heterocyclylene-(alkylene)-, bridged heterocyclylene, -(alkylene)-bridged heterocyclylene-, -bridged heterocyclylene-(alkylene)-, spiro heterocyclylene, -(alkylene)-spiro heterocyclylene-, or -spiro heterocyclylene-(alkylene), where each ring, by itself or as part of another group, is substituted with R^{m} and Rⁿ;
Z⁴ is alkylene, -(alkylene-NR″)—, —(NR″-alkylene)-, —O—, —NR″—, —(O-alkylene)_{d}-, -(alkylene-O)_{d}—, phenylene, heteroarylene, heterocyclylene, -(alkylene)-heterocyclylene-, -heterocyclylene-(alkylene)-, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p};
Z⁵ is phenylene, monocyclic heteroarylene, or heterocycylene, where each ring is substituted with R^{q} and R^{r}; and
Z⁶ is —S(O)₂; and
and each alkylene in Z³ and Z⁴, itself or as part of another group, is independently substituted with R^{s} and R^{t}.
C213a. In embodiment C213a, the compound of C213, or a pharmaceutically acceptable salt thereof, is wherein:
X¹, X², X³, X⁴, Z¹, and Z² are each a bond;
Z³ is-heterocyclylene-(alkylene)-, where heterocyclylene is substituted with R^{m} and Rⁿ and where alkylene is independently substituted with R^{s} and R^{t};
Z⁴ is phenylene or monocyclic heteroarylene, where each ring is substituted with R^{o} and R^{p};
Z⁵ is phenylene substituted with R^{q} and R^{r}; and
Z⁶ is —S(O)₂.
C214. In embodiment C214, the compound of any one of embodiments C1 to C177, C205 to C210, and C213, or a pharmaceutically acceptable salt thereof, is wherein:
Z³ is heterocyclylene, -(alkylene)-heterocyclylene-, -heterocyclylene-(alkylene)-, bridged heterocyclylene, -(alkylene)-bridged heterocyclylene-, -bridged heterocyclylene-(alkylene)-, spiro heterocyclylene, -(alkylene)-spiro heterocyclylene-, or -spiro heterocyclylene-(alkylene), where each ring, by itself or as part of another group, is substituted with R^{m} and Rⁿ;
Z⁴ is alkylene, —O—, heterocyclylene, -(alkylene)-heterocyclylene-, -heterocyclylene-(alkylene)-, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p} independently selected from hydrogen, deuterium, alkyl, alkoxy, halo, haloalkyl, haloalkoxy, cyano, hydroxy, amino, alkylamino, and dialkylamino;
Z⁵ is phenylene, monocyclic heteroarylene, or heterocycylene, where each ring is substituted with R^{q} and R^{r} independently selected from hydrogen, deuterium, alkyl, alkoxy, halo, haloalkyl, haloalkoxy, cyano, hydroxy, amino, alkylamino, and dialkylamino; and
Z⁶ is —S(O)₂; and
and each alkylene in Z³ and Z⁴, itself or as part of another group, is independently substituted with R^{s} and R^{t}.
C215. In embodiment C215, the compound of any one of embodiments C1 to C177, C205 to C210, and C213 to C214, or a pharmaceutically acceptable salt thereof, is wherein:
Z³ is heterocyclylene, bridged heterocyclylene, or spiro heterocyclylene, where each ring is substituted with R^{q} and R^{r};
Z⁴ is alkylene, —O—, heterocyclylene, -(alkylene)-heterocyclylene-, -(alkylene)-bridged heterocyclylene-, where each ring, by itself or as part of another group, is substituted with R^{o} and R^{p};
Z⁵ is phenylene, monocyclic heteroarylene, or heterocycylene, where each ring is substituted with R^{q} and R^{r}; and
Z⁶ is —S(O)₂; and
and each alkylene in Z⁴, itself or as part of another group, is substituted with R^{s} and R^{t}.
C216. In embodiment C216, the compound of any one of embodiments C1 to C193 and C195 to C215, or a pharmaceutically acceptable salt thereof, is wherein —Z⁵— is

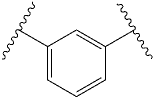 [SMILES: *c1cccc(*)c1]

(i.e., Z⁵ is phenylene where Z⁴ and Z⁶ are attached at meta position of the phenylene ring) substituted with R^{q} and R^{r} independently selected from hydrogen, deuterium, alkyl, alkoxy, halo, haloalkyl, and haloalkoxy.
C217. In embodiment C217, the compound of any one of embodiments C1 to C193 and C195 to C216, or a pharmaceutically acceptable salt thereof, is wherein —Z⁵— is

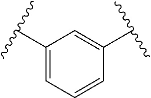 [SMILES: *c1cccc(*)c1]

substituted with R^{q} and R^{r} independently selected from hydrogen, deuterium, methyl, methoxy, fluoro, chloro, difluoromethyl, trifluoromethyl, difluoromethoxy, and trifluoromethoxy.
C218. In embodiment C218, the compound of any one of embodiments C1 to C193 and C195 to C217, or a pharmaceutically acceptable salt thereof, is wherein —Z⁵— is

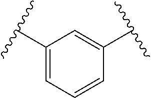 [SMILES: *c1cccc(*)c1]

substituted with R^{q} and R^{r} independently selected from hydrogen, deuterium, or fluoro.
C218a. In embodiment C218a, the compound of any one of embodiments C1 to C193 and C195 to C218, or a pharmaceutically acceptable salt thereof, is wherein —Z⁵— is

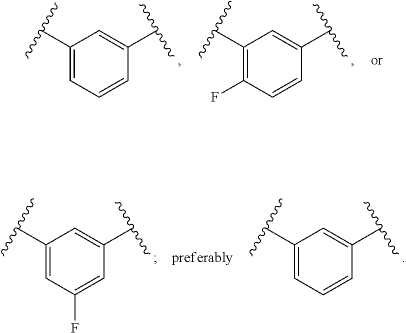 [SMILES: *c1cccc(*)c1 ; *c1ccc(F)c(*)c1 ; *c1cc(*)cc(F)c1 ; *c1cccc(*)c1]

C218b. In embodiment C218b, the compound of any one of embodiments C1 to C193 and C195 to C217, or a pharmaceutically acceptable salt thereof, is wherein —Z⁵— is

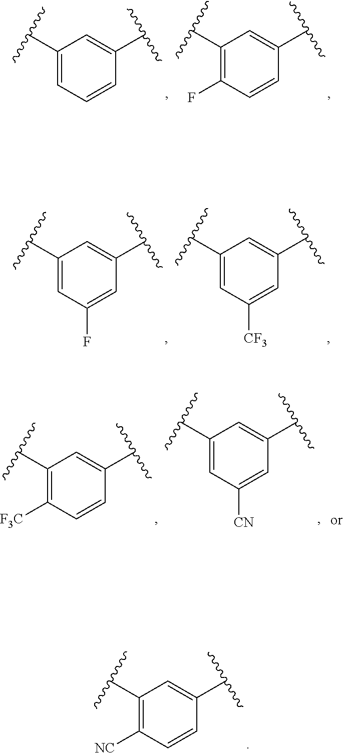

C219. In embodiment C219, the compound of any one of embodiments C1 to C193 and C195 to C210, C212 to C213, C214, and C215, or a pharmaceutically acceptable salt thereof, is wherein —Z⁵— is monocyclic heteroarylene (such as imidazol-1,5-diyl, pyridin-2,4-diyl, pyridin-2,6-diyl, or pyridin-3,5-diyl) substituted with R^{q} and R^{r} independently selected from hydrogen, alkyl, alkoxy, halo, haloalkyl, and haloalkoxy.
C220. In embodiment C220, the compound of any one of embodiments C1 to C193, C195 to C210, C212 to C213, C214, C215, and C219, or a pharmaceutically acceptable salt thereof, is wherein —Z⁵— is imidazol-2,5-diyl, pyridin-2,4-diyl, pyridin-2,6-diyl, or pyridin-3,5-diyl, each ring substituted with R^{q} and R^{r} independently selected from hydrogen, methyl, methoxy, fluoro, chloro, difluoromethyl, trifluoromethyl, 2,2,2-trifluoroethyl, difluoromethoxy, and trifluoromethoxy.
C221. In embodiment C221, the compound of any one of embodiments C1 to C193, C195 to C210, C212 to C213, C214, C215, C219, and C220, or a pharmaceutically acceptable salt thereof, is wherein —Z⁵— is imidazol-2,5-diyl, pyridin-2,4-diyl, pyridin-2,6-diyl, or pyridin-3,5-diyl, each ring substituted with R^{q} and R^{r} independently selected from hydrogen, methyl, methoxy, fluoro, chloro, difluoromethyl, trifluoromethyl, difluoromethoxy, and trifluoromethoxy.
C222. In embodiment C222, the compound of any one of embodiments C1 to C193, C195 to C198, C200, C203, C205 to C210, and C212 to C213, and C214 to C215, or a pharmaceutically acceptable salt thereof, is wherein —Z⁵— is heterocyclylene substituted with R^{q} and R^{r} independently selected from hydrogen, methyl, methoxy, fluoro, chloro, difluoromethyl, trifluoromethyl, 2,2,2-trifluoroethyl, difluoromethoxy, and trifluoromethoxy.
C223. In embodiment C223, the compound of any one of embodiments C1 to C193, C195 to C198, C200, C203, C205 to C210, C212 to C213, C214 to C215, and C222, or a pharmaceutically acceptable salt thereof, is wherein —Z⁵— is azetidinyl, pyrrolidinyl, piperazinyl, or piperidinyl.
C224. In embodiment C224, the compound of any one of embodiments C1 to C215, or a pharmaceutically acceptable salt thereof, is wherein each alkylene of Z¹, Z², Z³, Z⁴, Z⁵, and Z⁶, by itself and when present, is methylene, ethylene, or propylene, each substituted with R^{s} and R^{t}.
C225. In embodiment C225, the compound of any one of embodiments C1 to C215 and C224, or a pharmaceutically acceptable salt thereof, is wherein each alkylene of Z¹, Z′, Z³, Z⁴, Z⁵, and Z⁶, by itself and when present, is methylene substituted with R^{s} and R^{t}.
C226. In embodiment C226, the compound of any one of embodiments C1 to C215, or a pharmaceutically acceptable salt thereof, is wherein each alkylene of Z¹, Z², Z³, Z⁴, Z⁵, and Z⁶, by itself or as part of —(O-alkylene)ₐ- in Z¹, -(alkylene-O)ₐ— in Z¹, —(O-alkylene)_{b}- in Z², -(alkylene-O)_{b}— in Z², —(O-alkylene)_{c}- in Z³, -(alkylene-O)_{c}— in Z³, —(O-alkylene)_{d}- in Z⁴, and -(alkylene-O)_{d}— in Z⁴, and -(alkylene-O)— in Z⁶, and when present, is ethylene or propylene; as part of -(alkylene-NR″)— and —(NR″-alkylene)- and when present, is methylene, ethylene or propylene; and as part of -(alkylene)-cycloalkylene-, -cycloalkylene-(alkylene)-, -(alkylene)-phenylene-, -phenylene-(alkylene)-, -(alkylene)-monocyclic heteroarylene-, -monocyclic heteroarylene-(alkylene)-, -(alkylene)-heterocyclylene-, -heterocyclylene-(alkylene)-, -(alkylene)-bridged heterocyclylene-, -bridged heterocyclylene-(alkylene)-, -(alkylene)-spiro heterocyclylene-, and -spiro heterocyclylene-(alkylene)- is methylene, ethylene, propylene, or butylene, preferably methylene, wherein each of above alkylene group is substituted with R^{s} and R^{t}.
C227. In embodiment C227, the compound of any one of embodiments C1 to C215 and C226, or a pharmaceutically acceptable salt thereof, is wherein each alkylene of Z¹, Z², Z³, Z⁴, Z⁵, and Z⁶, by itself or as part of —(O-alkylene)ₐ- in Z¹, and -(alkylene-O)ₐ— in Z¹, —(O-alkylene)_{b}- in Z², -(alkylene-O)_{b}— in Z², —(O-alkylene)_{c}- in Z³, -(alkylene-O)_{c}— in Z³, —(O-alkylene)ₐ- in Z⁴, and -(alkylene-O)_{d}— in Z⁴, and -(alkylene-O)— in Z⁶, and when present, is ethylene; as part of -(alkylene-NR″)—) and —(NR″-alkylene)- and when present, is methylene; and as part of -(alkylene)-cycloalkylene-, -cycloalkylene-(alkylene)-, -(alkylene)-phenylene-, -phenylene-(alkylene)-, -(alkylene)-monocyclic heteroarylene-, -monocyclic heteroarylene-(alkylene)-, -(alkylene)-heterocyclylene-, -heterocyclylene-(alkylene)-, -(alkylene)-bridged heterocyclylene-, -bridged heterocyclylene-(alkylene)-, -(alkylene)-spiro heterocyclylene-, and -spiro heterocyclylene-(alkylene)- and when present, is methylene.
C228. In embodiment C228, the compound of any one of embodiments C1 to 227 or a pharmaceutically acceptable salt thereof, is wherein each R, R′ and R″ of Z¹, Z², Z³, Z⁴, Z⁵, and Z⁶, when present, is independently hydrogen or methyl.
C229. In embodiment C229, the compound of any one of embodiments C1 to C228, or a pharmaceutically acceptable salt thereof, is wherein each R, R′ and R″ of Z¹, Z², Z³, Z⁴, Z⁵, and Z⁶, when present, is hydrogen.
C230. In embodiment C230, the compound of any one of embodiments C1 to C228, or a pharmaceutically acceptable salt thereof, is wherein each R, R′ and R″ of Z¹, Z², Z³, Z⁴, Z⁵, and Z⁶, when present, is methyl.
C231. In embodiment C231 the compound of any one of embodiments C1 to C230, or a pharmaceutically acceptable salt thereof, is wherein each cycloalkylene of Z², Z³, and Z⁴, when present, is independently selected from cyclopropylene, cyclobutylene, cyclopentylene, and cyclohexylene.
C232. In embodiment C232, the compound of any one of embodiments C1 to C231, or a pharmaceutically acceptable salt thereof, is wherein each cycloalkylene of Z², Z³, and Z⁴, when present, is independently selected from 1,3-cyclopentylene, 1,3-cyclohexylene, and 1,4-cyclohexylene.
C233. In embodiment C233, the compound of any one of embodiments C1 to C232, or a pharmaceutically acceptable salt thereof, is wherein heteroarylene is monocyclic heteroarylene and each monocyclic heteroarylene of Z¹, Z³, Z⁴, and Z⁵, when present, is independently selected from imidazoldiyl, pyridindiyl and pyrimidindiyl unless stated otherwise in any of the embodiments above.
C234. In embodiment C234, the compound of any one of embodiments C1 to C233, or a pharmaceutically acceptable salt thereof, is wherein heteroarylene is monocyclic heteroarylene and each monocyclic heteroarylene of Z¹, Z³, Z⁴, and Z⁵, when present, is independently selected from imidazol-2,5-diyl, pyridin-2,4-diyl, pyridin-2,6-diyl, and pyridin-3,5-diyl, unless stated otherwise in any of the embodiments above.
C235. In embodiment C235, the compound of any one of embodiments C1 to C234, or a pharmaceutically acceptable salt thereof, is wherein each phenylene of Z¹, Z³, Z⁴, and Z⁵, when present, is independently selected from 1,3-phenylene and 1,4-phenylene unless stated otherwise in any of the embodiments above.
C236. In embodiment C236, the compound of any one of embodiments C1 to C235, or a pharmaceutically acceptable salt thereof, is wherein each heterocyclylene, bridged heterocyclylene, and spiro heterocyclylene, of Z¹, Z², Z³, Z⁴, and Z⁵, when present, are independently selected from:

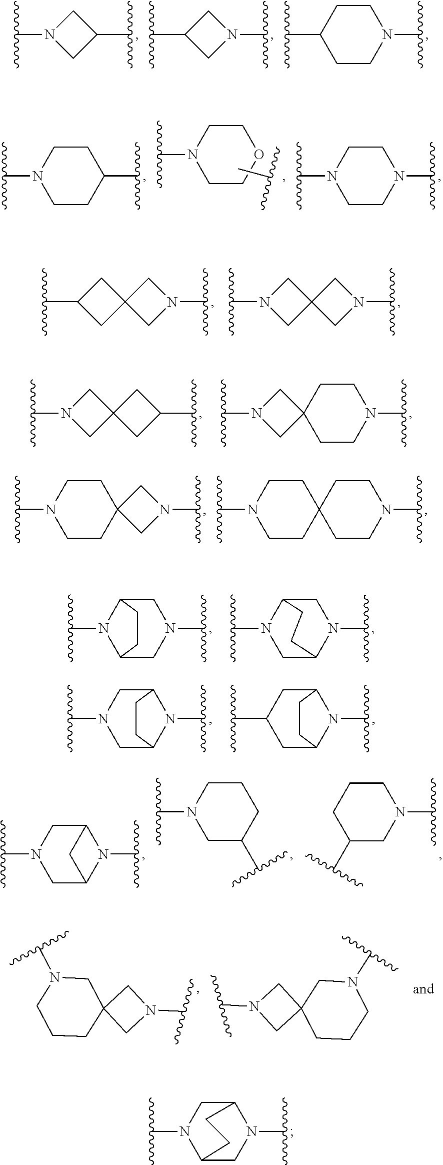

wherein each ring is optionally substituted with 1, 2, or 3 fluoro, unless stated otherwise in any of the embodiments above.
C237. In embodiment C237, the compound of any one of embodiments C1 to C236, or a pharmaceutically acceptable salt thereof, is wherein each heterocyclylene, bridged heterocyclylene, and spiro heterocyclylene, of Z¹, Z², Z³, Z⁴, and Z⁵, when present, are independently selected from:

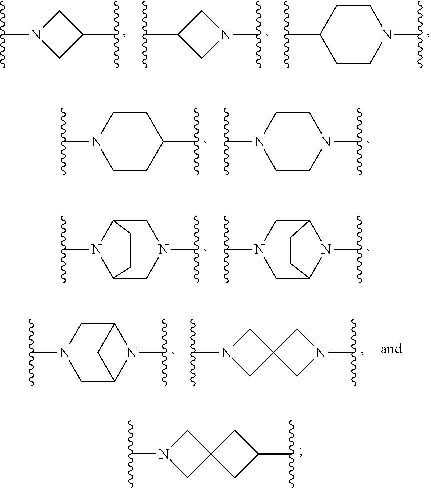

wherein each ring is optionally substituted with 1 or 2 fluoro, unless stated otherwise in any of the embodiments above.
C238. In embodiment C238, the compound of any one of embodiments C1 to C177, or a pharmaceutically acceptable salt thereof, is wherein L (when the Degron is a group of formula (iii) to (vi)), —X¹-L-, —X²-L-, —X³-L- and —X⁴-L- (when the Degron is a group of formula (i) or (ii)) are independently:

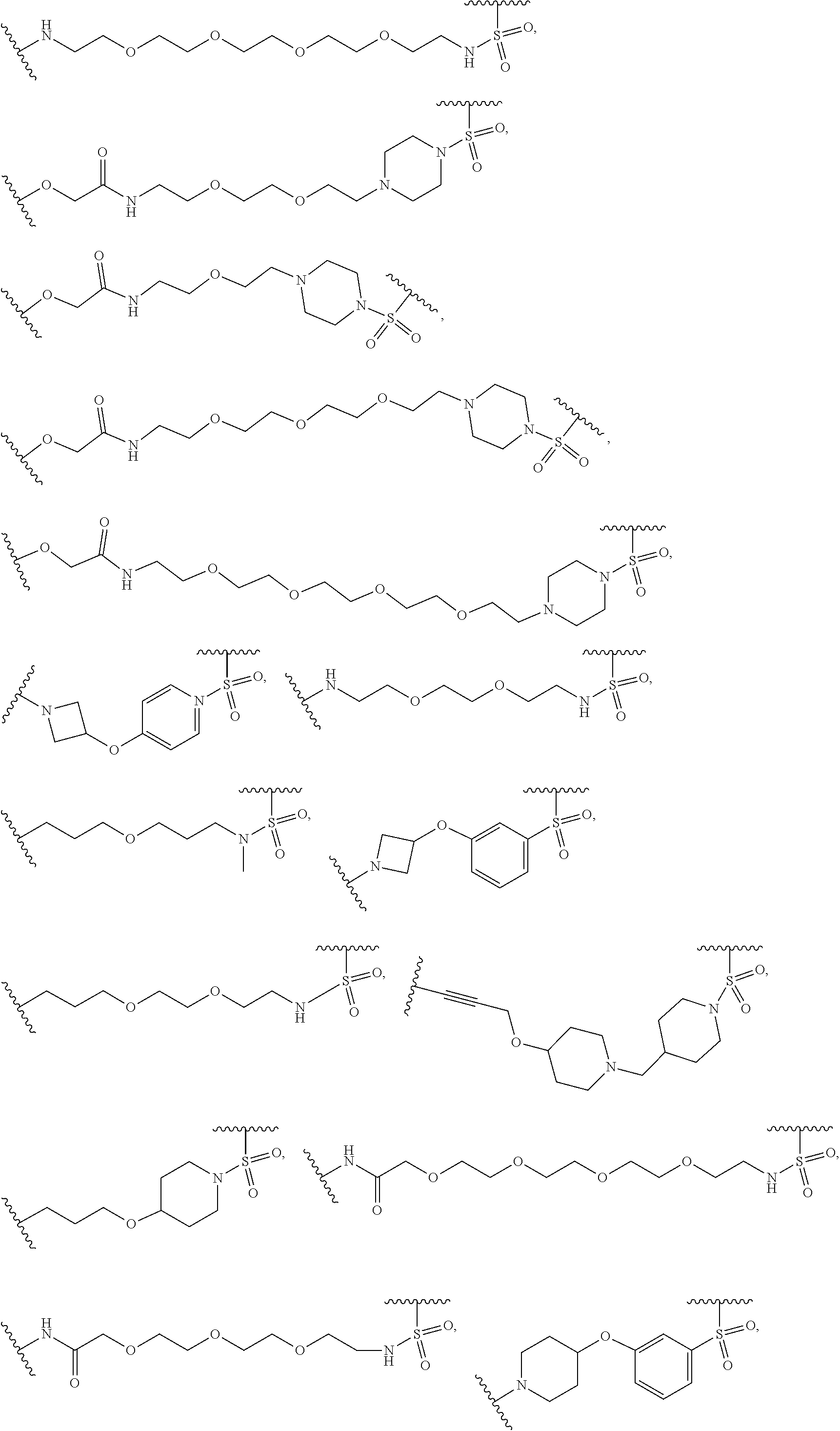

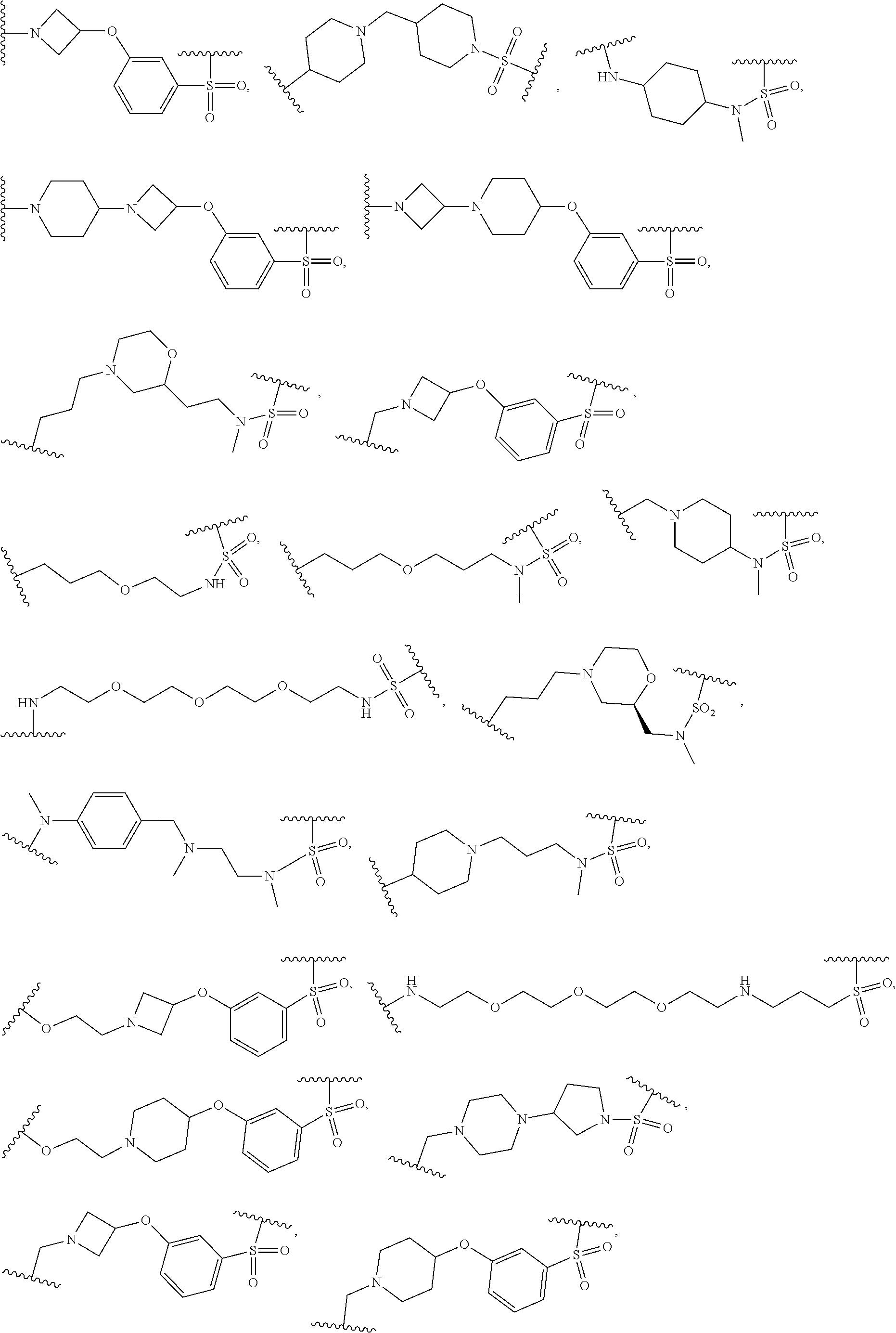

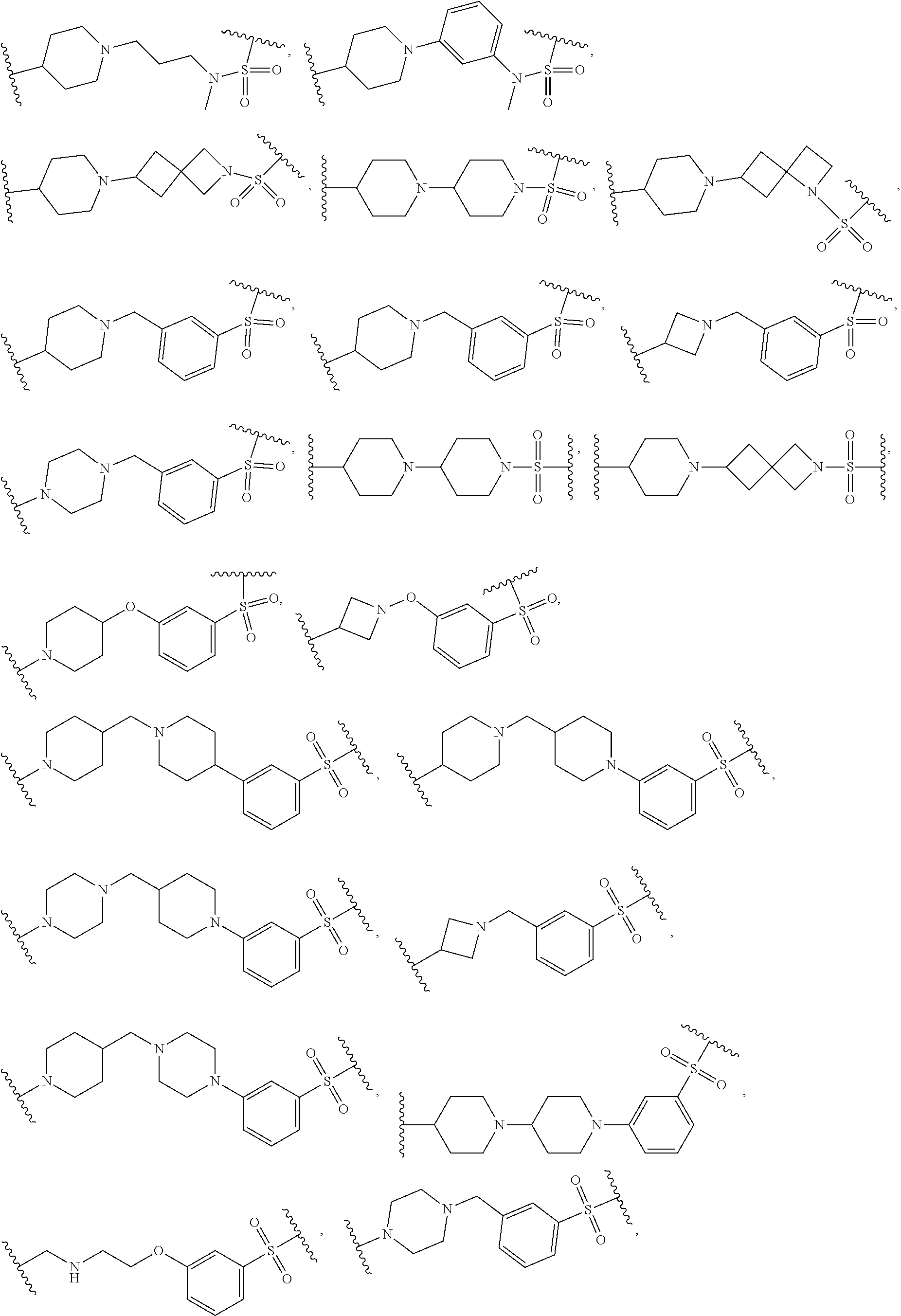

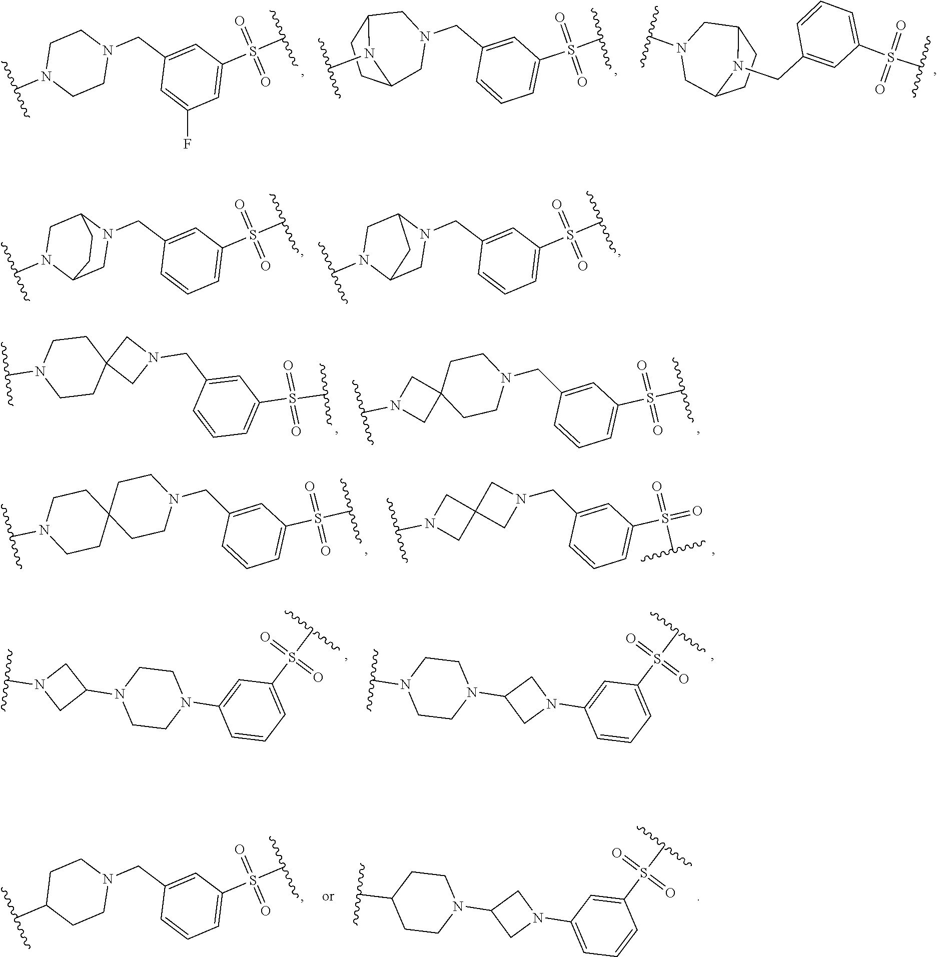

C239. In embodiment C239, the compound of any one of embodiments C1 to C177, or a pharmaceutically acceptable salt thereof, is wherein L (when the Degron is a group of formula (iii) to (vi)), —X¹-L-, —X²-L-, —X³-L-, and —X⁴-L- (when the Degron is a group of formula (i) or (ii)) are independently:

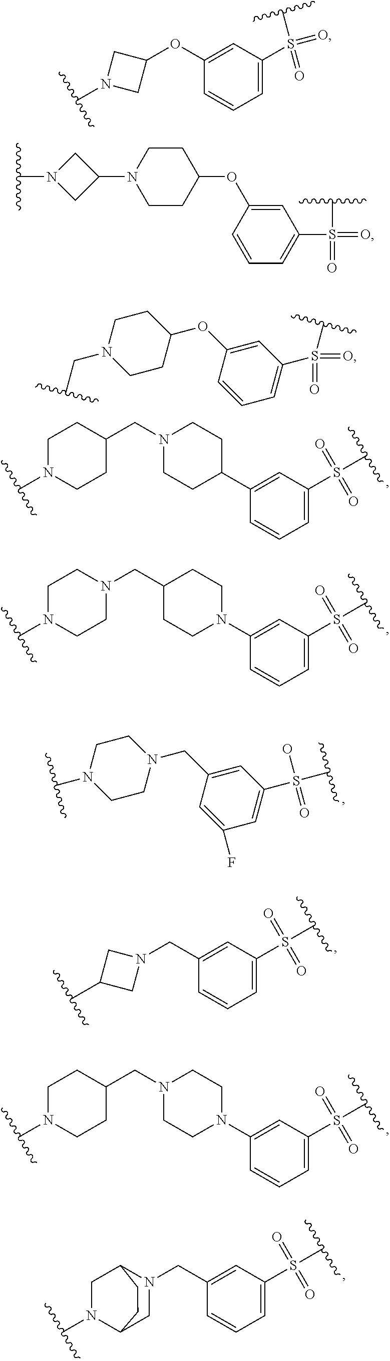

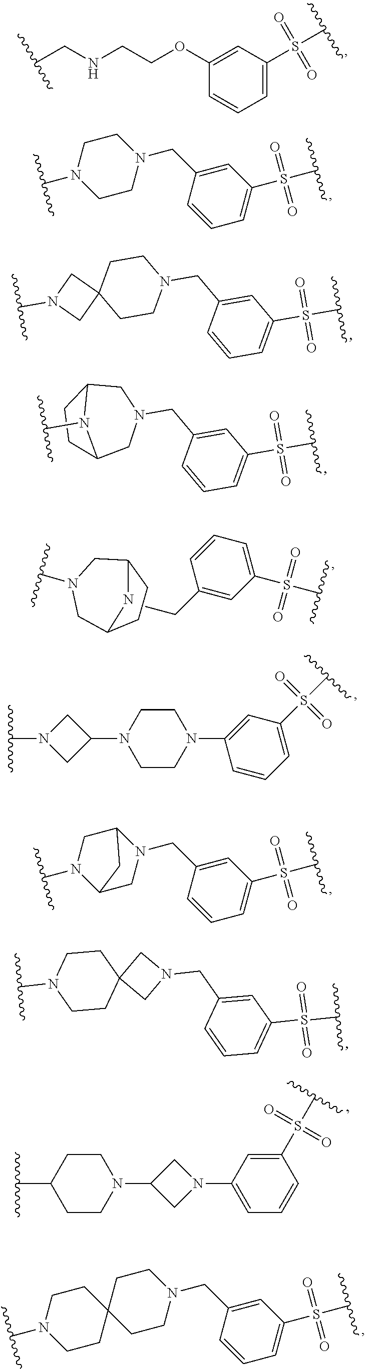

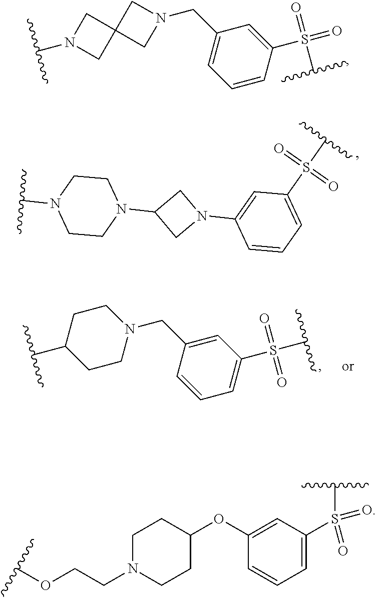 [SMILES: *N1CC2(C1)CN(Cc1cccc(S(*)(=O)=O)c1)C2 ; *N1CCN(C2CN(c3cccc(S(*)(=O)=O)c3)C2)CC1 ; *C1CCN(Cc2cccc(S(*)(=O)=O)c2)CC1 ; *OCCN1CCC(Oc2cccc(S(*)(=O)=O)c2)CC1]

C240. In embodiment C240, the compound of any one of embodiments C1 to C193, C195 to C210, C212, C213, C214, and C215, or a pharmaceutically acceptable salt thereof, is wherein —Z³—Z⁴—Z⁵—Z⁶— is:

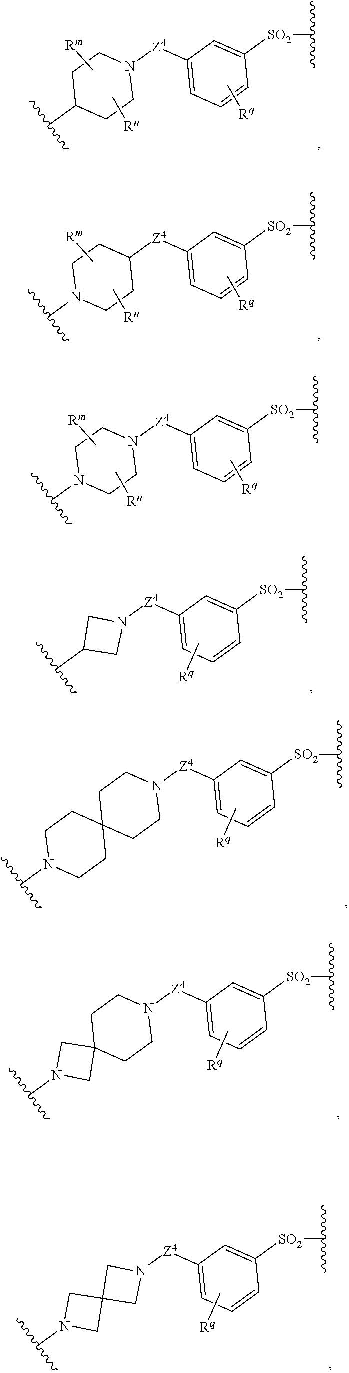

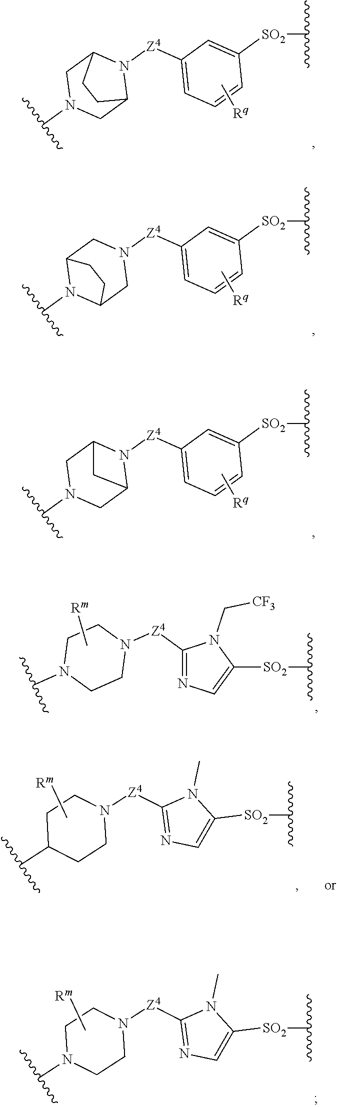

wherein each R^{m}, Rⁿ, and R^{q} are independently selected from hydrogen, alkyl, halo, haloalkyl, haloalkoxy, alkoxy, and cyano, preferably each R^{q} and R^{r} are independently selected from hydrogen, methyl, fluoro, chloro, cyano, methoxy, difluoromethoxy, difluoromethyl, and trifluoromethyl.
C241. In embodiment C241, the compound of any one of embodiments C1 to C193, C195 to C210, C212, C213, C214, C215, and C240, or a pharmaceutically acceptable salt thereof, is wherein —Z³—Z⁴—Z⁵—Z⁶— is:

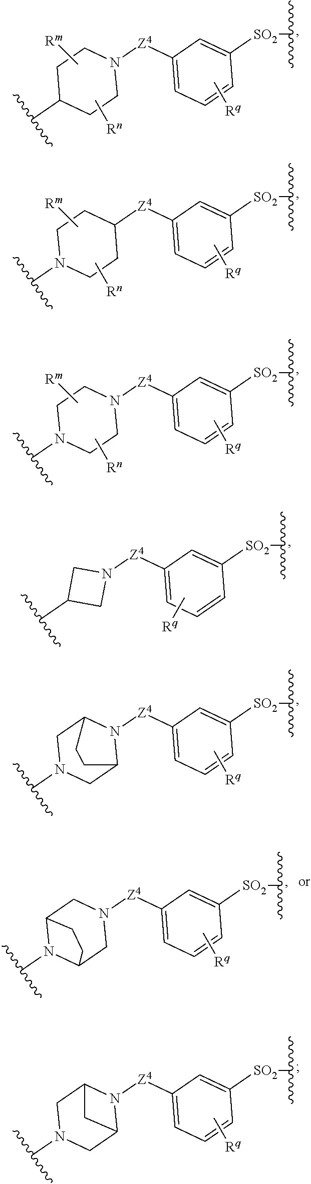

wherein each R^{m}, Rⁿ, and R^{q} are independently selected from hydrogen, alkyl, halo, haloalkyl, haloalkoxy, alkoxy, hydroxy and cyano (i.e., R^{r} is hydrogen).
C242. In embodiment C242, the compound of any one of embodiments C1 to C193, C195 to C210, C212, C213, C214, C215, and C241, or a pharmaceutically acceptable salt thereof, is wherein —Z³—Z⁴—Z⁵—Z⁶— is:

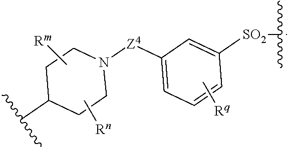

C243. In embodiment C243, the compound of any one of embodiments C1 to C193, C195 to C210, C212, C213, C214, C215, and C241, or a pharmaceutically acceptable salt thereof, is wherein —Z³—Z⁴—Z⁵—Z⁶— is:

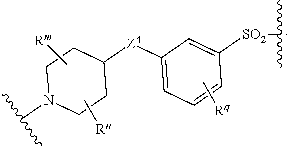

C244. In embodiment C244, the compound of any one of embodiments C1 to C193, C195 to C210, C212, C213, C214, C215, and C241, or a pharmaceutically acceptable salt thereof, is wherein —Z³—Z⁴—Z⁵—Z⁶— is:

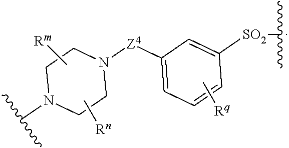

C245. In embodiment C245, the compound of any one of embodiments C1 to C193, C195 to C210, C212, C213, C214, C215, and C241, or a pharmaceutically acceptable salt thereof, is wherein —Z³—Z⁴—Z⁵—Z⁶— is:

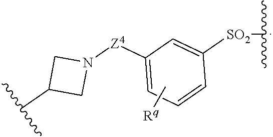

C246. In embodiment C246, the compound of any one of embodiments C1 to C193, C195 to C210, C212, C213, C214, C215, and C241, or a pharmaceutically acceptable salt thereof, is wherein —Z³—Z⁴—Z⁵—Z⁶— is:

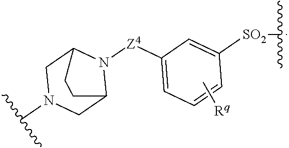

C247. In embodiment C247, the compound of any one of embodiments C1 to C193, C195 to C210, C212, C213, C214, C215, and C241, or a pharmaceutically acceptable salt thereof, is wherein —Z³—Z⁴—Z⁵—Z⁶— is:

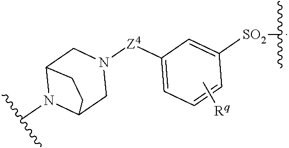

C248a. In embodiment C248a, the compound of any one of embodiments C1 to C193, C195 to C210, C212, C213, C214, C215, and C241, or a pharmaceutically acceptable salt thereof, is wherein —Z³—Z⁴—Z⁵—Z⁶— is:

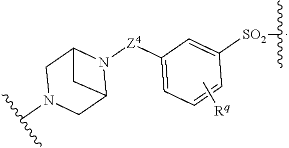

C248. In embodiment C248, the compound of any one of embodiments C240 to C248a, or a pharmaceutically acceptable salt thereof, is wherein

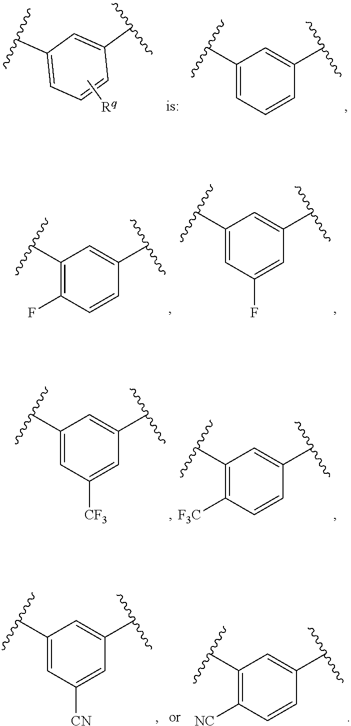

C249. In embodiment C249, the compound of any one of embodiments C1 to C178, C191 to C193, C206, and C208 to C211, or a pharmaceutically acceptable salt thereof, is wherein —Z³—Z⁴—Z⁵—Z⁶— is:

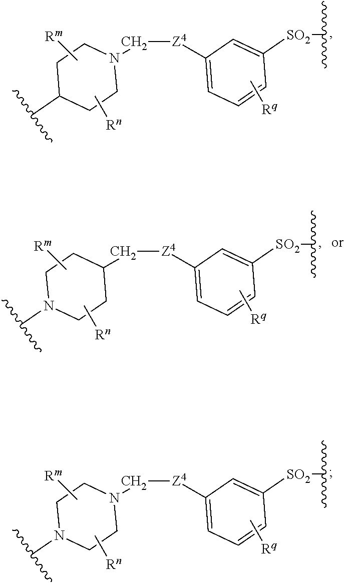

wherein each R^{m}, Rⁿ, and R^{q} are independently selected from hydrogen, alkyl, halo, haloalkyl, haloalkoxy, alkoxy, hydroxy and cyano (i.e., R^{r} is hydrogen).
C250. In embodiment C250, the compound of any one of embodiments C1 to C178, C191 to C193, C206, C208 to C211, and C249, or a pharmaceutically acceptable salt thereof, is wherein —Z³—Z⁴—Z⁵—Z⁶— is:

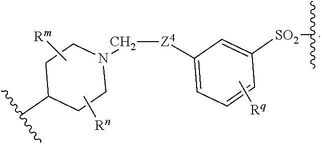

C251. In embodiment C251, the compound of any one of embodiments C1 to C178, C191 to C193, C206, C208 to C211, and C249, or a pharmaceutically acceptable salt thereof, is wherein —Z³—Z⁴—Z⁵—Z⁶— is:

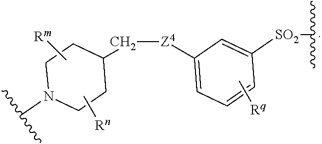

C252. In embodiment C252, the compound of any one of embodiments C1 to C178, C191 to C193, C206, C208 to C211, and C249, or a pharmaceutically acceptable salt thereof, is wherein —Z³—Z⁴—Z⁵—Z⁶— is:

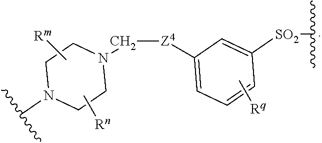

C253. In embodiment C253, the compound of any one of embodiments C1 to C178, C191 to C193, C206, and C208 to C211, or a pharmaceutically acceptable salt thereof, is wherein —Z³—Z⁴—Z⁵—Z⁶— is:

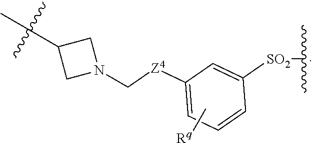

C254. In embodiment C254, the compound of any one of embodiments C249 to C253, or a pharmaceutically acceptable salt thereof, is wherein

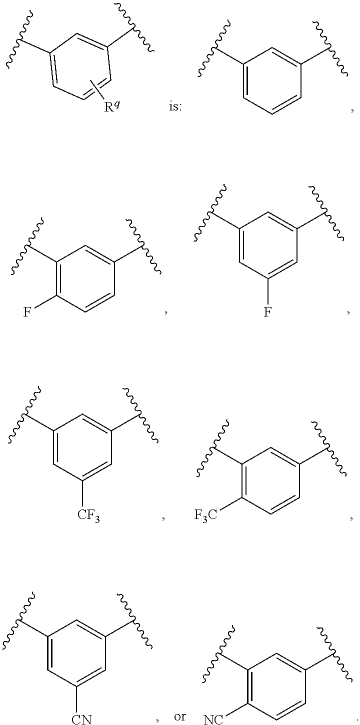

C255. In embodiment C255, the compound of any one of embodiments C1 to C201, C205 to C210, C212 to C213, C214 to C223, and C240 to C248, or a pharmaceutically acceptable salt thereof, is wherein Z⁴ is alkylene substituted with R^{s} and R^{t} where R^{s} and R^{t} are hydrogen.
C256 In embodiment C256, the compound of any one of embodiments C1 to C201, C205 to C210, C212 to C213, C214 to C233 (in some embodiments C214 to C223), C240 to C248, and C255, or a pharmaceutically acceptable salt thereof, is wherein the alkylene of Z⁴ is —CH₂—, —(CH₂)₂—, —(CH₂)₃—, —CH(CH₃)—, —CH₂—CH(CH₃)—CH₂—, or —CH₂—C(CH₃)₂—CH₂—.
C257. In embodiment C257, the compound of any one of embodiments C1 to C201, C205 to C210, C212 to C213, C214 to C223, C240 to C248, C255, and C256, or a pharmaceutically acceptable salt thereof, is wherein the alkylene of Z⁴ is —CH₂—.
C258. In embodiment C258, the compound of any one of embodiments C1 to C201, C205 to C210, C212 to C213, C214 to C233 (in some embodiments C214 to C223), and C240 to C248, or a pharmaceutically acceptable salt thereof, is wherein Z⁴ is —O—.
C259. In embodiment C259, the compound of any one of embodiments C1 to C201, C205 to C210, C212 to C213, C214 to C233 (in some embodiments C214 to C223), and C240 to C248, or a pharmaceutically acceptable salt thereof, is wherein Z⁴ is alkylene substituted with R^{s} and R^{t} where R^{s} is hydrogen or deuterium and R′ is hydrogen, deuterium, haloalkyl, hydroxy, alkoxy, or cyano.
C260. In embodiment C260, the compound of any one of embodiments C1 to C201, C205 to C210, C212 to C213, C214 to C233 (in some embodiments C214 to C223), C240 to C248, and C259, or a pharmaceutically acceptable salt thereof, is wherein Z⁴ is alkylene substituted with R^{s} and R^{t} where R^{s} is hydrogen or deuterium and R^{t} is hydrogen and deuterium.
C261. In embodiment C261, the compound of any one of embodiments C1 to C201, C205 to C210, C212 to C213, C214 to C233 (in some embodiments C214 to C223), C240 to C248, and C259, or a pharmaceutically acceptable salt thereof, is wherein Z⁴ is alkylene substituted with R^{s} and R^{t} where R^{s} is hydrogen and R^{t} is haloalkyl.
C262. In embodiment C262, the compound of any one of embodiments C1 to C201, C205 to C210, C212 to C213, C214 to C233 (in some embodiments C214 to C223), C240 to C248, and C259, or a pharmaceutically acceptable salt thereof, is wherein Z⁴ is alkylene substituted with R^{s} and R^{t} where R^{s} is hydrogen and R^{t} is hydroxy.
C263. In embodiment C263, the compound of any one of embodiments C1 to C201, C205 to C210, C212 to C213, C214 to C233 (in some embodiments C214 to C223), C240 to C248, and C259, or a pharmaceutically acceptable salt thereof, is wherein Z⁴ is alkylene substituted with R^{s} and R^{t} where R^{s} is hydrogen and R^{t} is alkoxy.
C264. In embodiment C264, the compound of any one of C1 to C201, C205 to C210, C212 to C213, C214 to C233 (in some embodiments C214 to C223), C240 to C248, C259, and C261 to C263, or a pharmaceutically acceptable salt thereof, is wherein Z⁴ is —CH(CHF₂)—, —CH(CF₃)—, —C(CH₃)(CF₃)—, —CH(CH₂CF₃)—, —CH(CH₂CH₂CF₃)—, —CH(CH(CF₃)₂)—, —CH(CH₂OH)—, —CH(CH₂OCH₃)—, —CH(CH₂O-ethyl)-, or —CH(CH₂CN)—, —CH₂—CH(CF₃)—CH₂—, —CH₂—CH(OH)—CH₂—, or —CH₂—CH(OCH₃)—CH₂—.
C265. In embodiment C265, the compound of any one of C1 to C194, C205 to C210, C212 to C213, C214 to C227, and C240 to C248, or a pharmaceutically acceptable salt thereof, is wherein Z⁴ is -(alkylene)-heterocyclylene-, where heterocyclylene is substituted with R^{o} and R^{p}.
C266. In embodiment C266, the compound of any one of C1 to C194, C205 to C210, C212 to C213, C214 to C227, C240 to C248, and C265, or a pharmaceutically acceptable salt thereof, is wherein Z⁴ is —(CH₂)-heterocyclylene- where heterocyclylene is substituted with R^{o} and R^{p}.
C267. In embodiment C267, the compound of any one of C1 to C194, C205 to C210, C212 to C213, C214 to C227, C240 to C248, C265, and C266, or a pharmaceutically acceptable salt thereof, is wherein Z⁴ is:

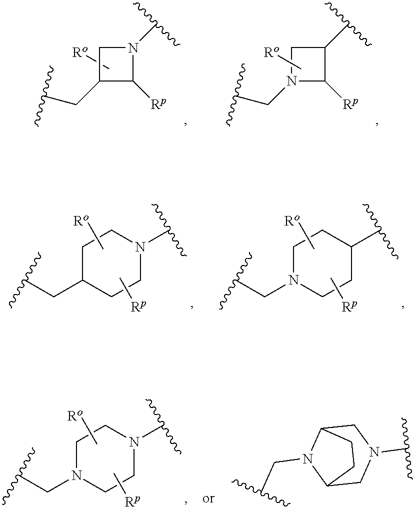

C268. In embodiment C268, the compound of any one of C1 to C201, C205 to C210, C212, C213, C214 to C218b, C240 to C254, and C265 to C267 or a pharmaceutically acceptable salt thereof, is wherein —Z³—Z⁴—Z⁵—Z⁶— is:

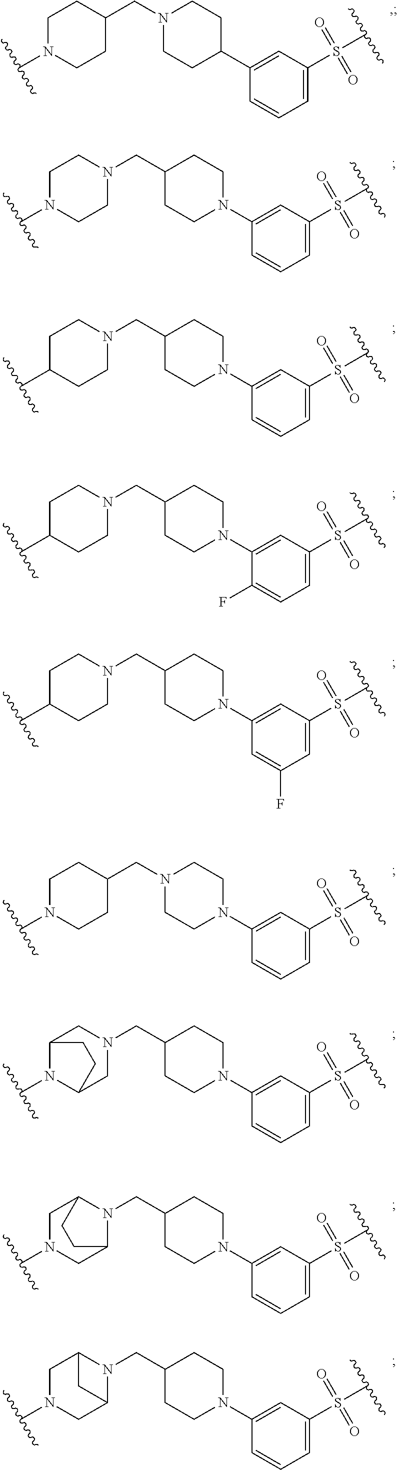

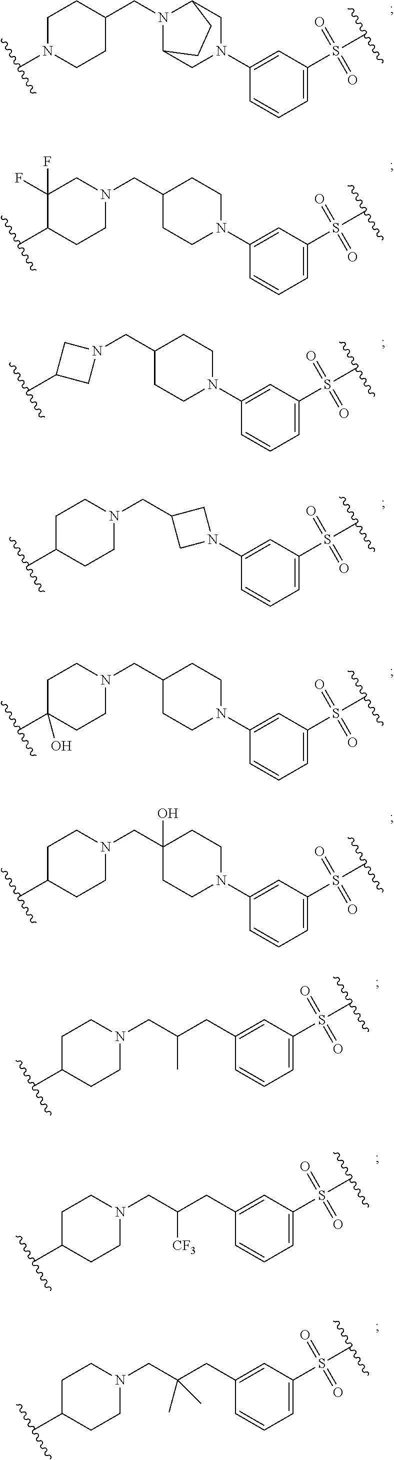

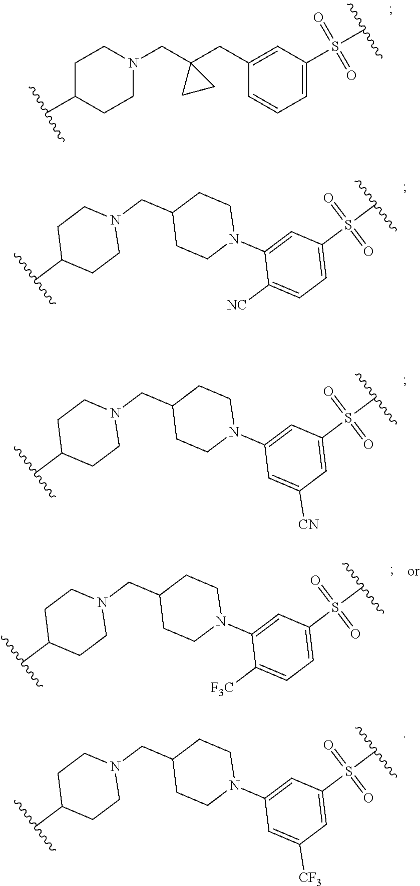 [SMILES: *C1CCN(CC2(Cc3cccc(S(*)(=O)=O)c3)CC2)CC1 ; *C1CCN(CC2CCN(c3cc(S(*)(=O)=O)ccc3C#N)CC2)CC1 ; *C1CCN(CC2CCN(c3cc(C#N)cc(S(*)(=O)=O)c3)CC2)CC1 ; *C1CCN(CC2CCN(c3cc(S(*)(=O)=O)ccc3C(F)(F)F)CC2)CC1 ; *C1CCN(CC2CCN(c3cc(C(F)(F)F)cc(S(*)(=O)=O)c3)CC2)CC1]

C269. In embodiment C269, the compound of any one of C1 to C201, C205 to C210, C212, C213, C214 to C218b, C240 to C254, and C265 to C268, or a pharmaceutically acceptable salt thereof, is wherein —Z³—Z⁴—Z⁵—Z⁶— is:

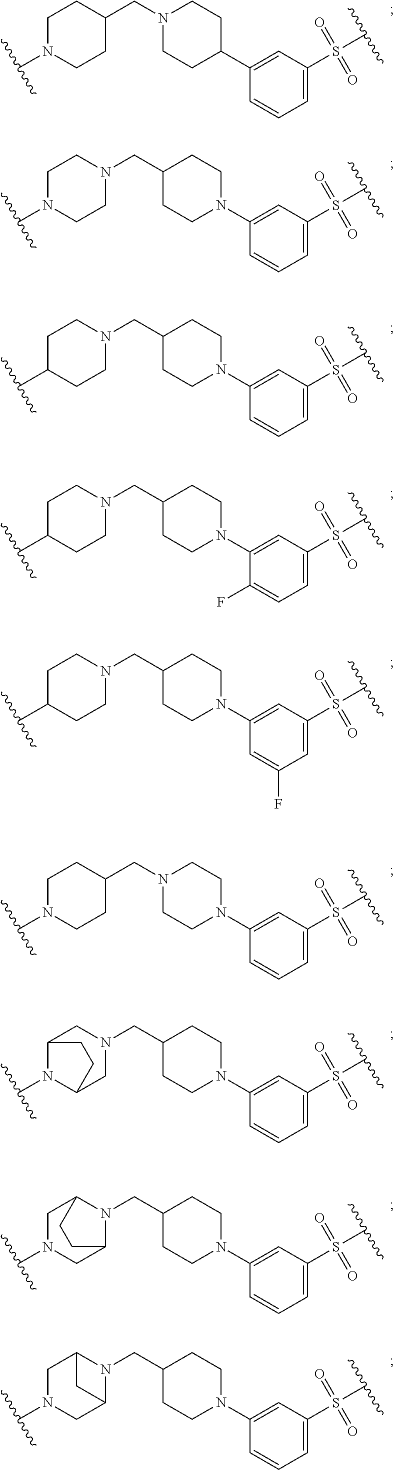

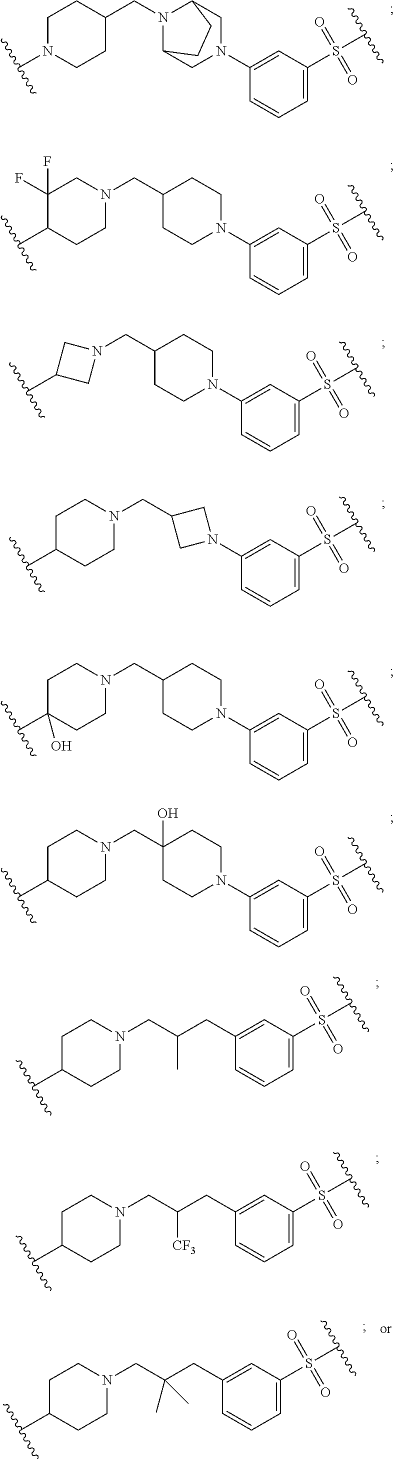

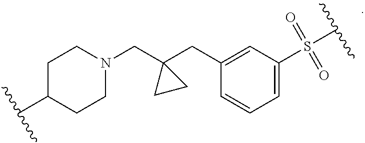 [SMILES: *C1CCN(CC2(Cc3cccc(S(*)(=O)=O)c3)CC2)CC1]

C270. In embodiment C270, the compound of any one of C1 to C201, C205 to C210, C212, C213, C214 to C218b, C240 to C254, and C265 to C269 or a pharmaceutically acceptable salt thereof, is wherein —Z³—Z⁴—Z⁵—Z⁶— is:

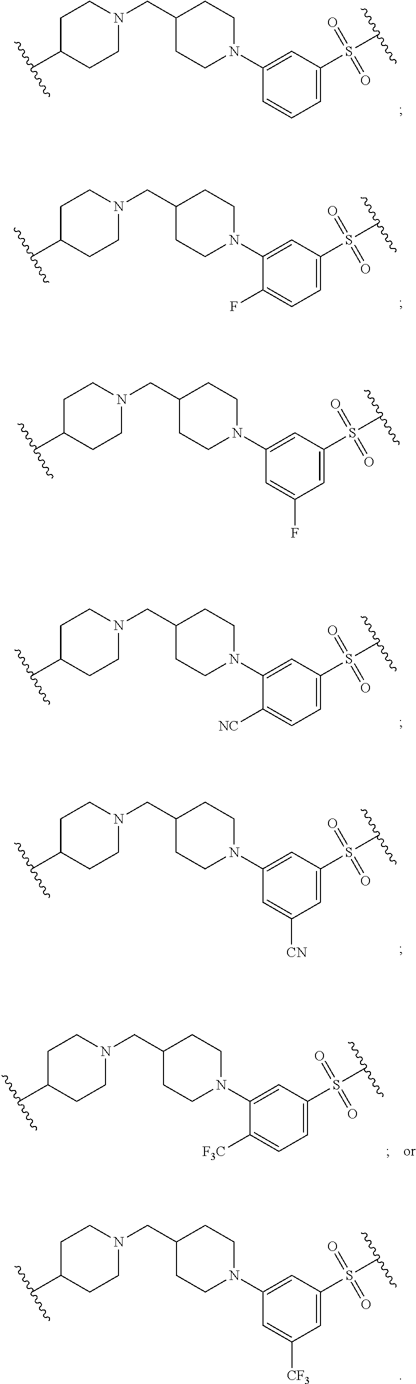

C271. In embodiment C271, the compound of any one of C1 to C201, C205 to C210, C212, C213, C214 to C218b, C240 to C254, and C265 to C270 or a pharmaceutically acceptable salt thereof, is wherein —Z³—Z⁴—Z⁵—Z⁶— is:

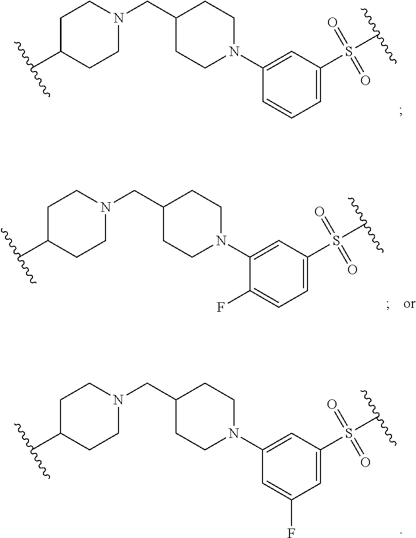 [SMILES: *C1CCN(CC2CCN(c3cccc(S(*)(=O)=O)c3)CC2)CC1 ; *C1CCN(CC2CCN(c3cc(S(*)(=O)=O)ccc3F)CC2)CC1 ; *C1CCN(CC2CCN(c3cc(F)cc(S(*)(=O)=O)c3)CC2)CC1]

C272. In embodiment C272, the compound of any one of C1 to C177, or a pharmaceutically acceptable salt thereof, is wherein L (when the Degron is a group of formula (iii) to (vi)), —X¹-L-, —X²-L-, —X³-L- and —X⁴-L- are independently:

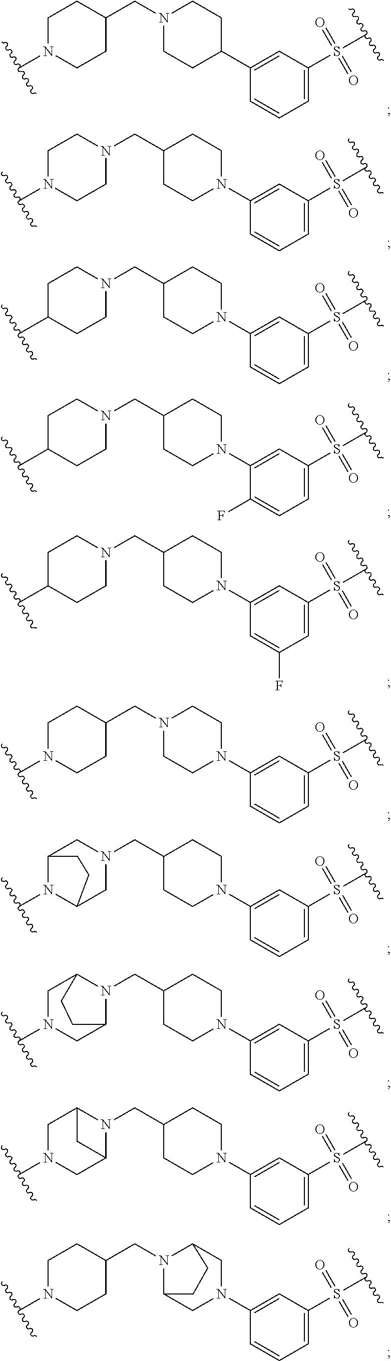

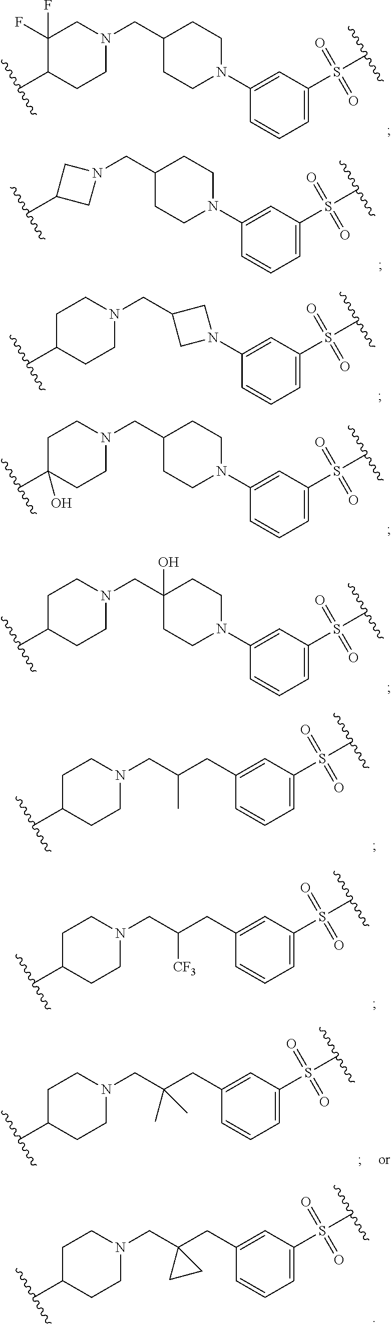

C273. In embodiment C273, the compound of any one of C1 to C177, and C272, or a pharmaceutically acceptable salt thereof, is wherein L (when the Degron is a group of formula (iii) to (vi)), —X¹-L-, —X²-L-, —X³-L- and —X⁴-L- are independently:

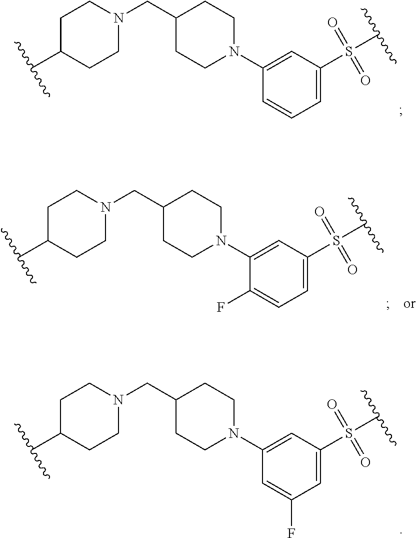 [SMILES: *C1CCN(CC2CCN(c3cccc(S(*)(=O)=O)c3)CC2)CC1 ; *C1CCN(CC2CCN(c3cc(S(*)(=O)=O)ccc3F)CC2)CC1 ; *C1CCN(CC2CCN(c3cc(F)cc(S(*)(=O)=O)c3)CC2)CC1]

C274. In embodiment C274, the compound of any one of embodiments C1 to C173 and C178 to C273, or a pharmaceutically acceptable salt thereof, is wherein Degron is the E3 ubiquitin ligase ligand selected from:

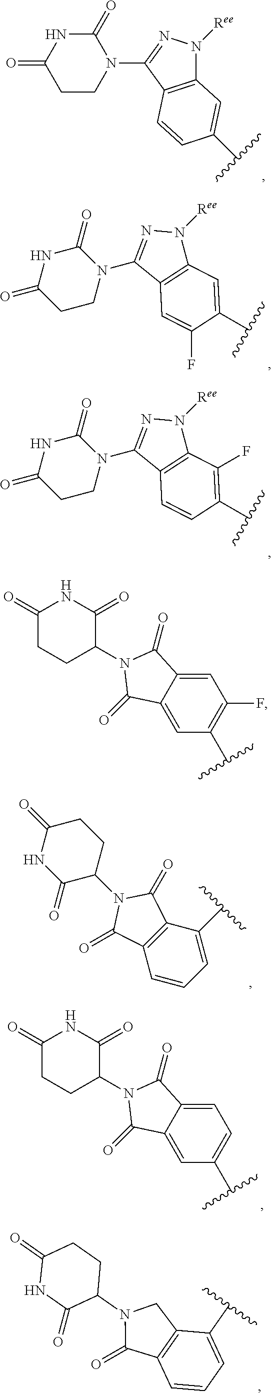

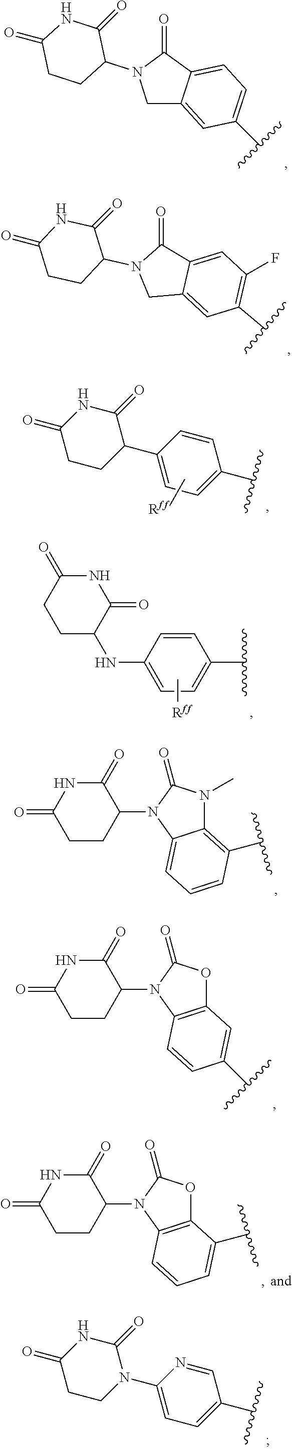

where R^{ee} is hydrogen, methyl, ethyl, cyclopropyl, or 2,2,2-trifluoroethyl and R^{ff} is hydrogen, methyl, cyclopropyl, fluoro, cyano, methoxy, difluoromethoxy, trifluoromethoxy, or trifluoromethyl.
C275. In embodiment C275, the compound of any one of embodiments C1 to C173 and C178 to C274, or a pharmaceutically acceptable salt thereof, is wherein Degron is the E3 ubiquitin ligase ligand selected from:

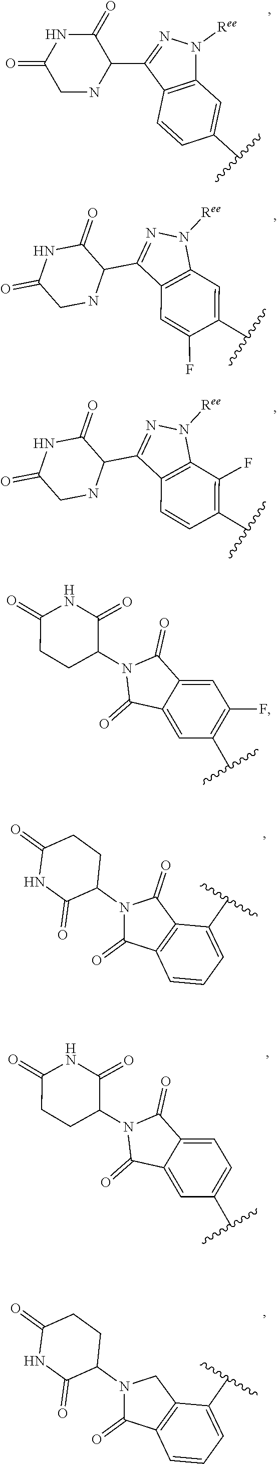

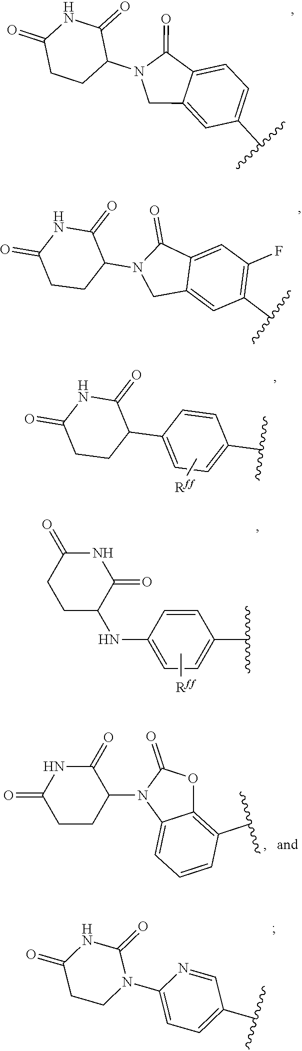

where R^{ee} is hydrogen, methyl, ethyl, cyclopropyl, or 2,2,2-trifluoroethyl and R^{ff} is hydrogen, methyl, cyclopropyl, fluoro, cyano, methoxy, difluoromethoxy, trifluoromethoxy, or trifluoromethyl.
C276. In embodiment C276, the compound of any one of embodiments C1 to C173 and C178 to C275, or a pharmaceutically acceptable salt thereof, is wherein Degron is the E3 ligase ligand selected from:

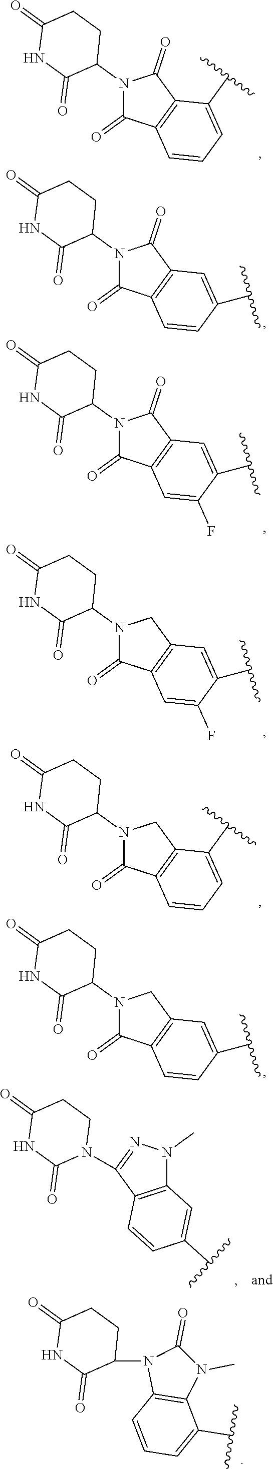

C277. In embodiment C277, the compound of any one of embodiments C1 to C173 and C178 to C275, or a pharmaceutically acceptable salt thereof, is wherein Degron is the E3 ubiquitin ligase ligand is

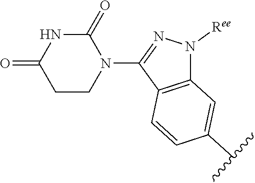

where each R^{ee} is hydrogen, methyl, ethyl, cyclopropyl, or 2,2,2-trifluoroethyl (in an embodiment methyl) and each R^{ff}, when specifically drawn is hydrogen, methyl, cyclopropyl, fluoro, cyano, methoxy, difluoromethoxy, trifluoromethoxy, or trifluoromethyl.
C278. In embodiment C278, the compound of any one of embodiments C1 to C173 and C178 to C273, or a pharmaceutically acceptable salt thereof, is wherein Degron is the E3 ubiquitin ligase ligand is

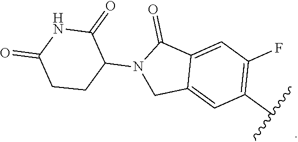 [SMILES: *c1cc2c(cc1F)C(=O)N(C1CCC(=O)NC1=O)C2]

C279. In embodiment C279, the compound of any one of embodiments C1 to C273, or a pharmaceutically acceptable salt thereof, is wherein R^{x} and R^{x1} are each hydrogen.

For sake of clarity, when an embodiment refers to more than one preceding embodiment of varying scopes, only those groups that fall within the scope of group(s) recited in a preceding embodiment(s) should be selected from the embodiment referring thereto. For example, of the groups recited in embodiment C7, while all the recited groups in C7 should be selected for embodiment C1, only fluoro, chloro, and bromo should be selected for embodiment C4 as scope of R¹ in C4 is limited to halo; and only difluoromethyl, trifluoromethyl, difluoroethyl, and trifluoroethyl should be selected for embodiment C5 as scope of R¹ in C5 is limited to haloalkyl.

Additional embodiments are provided below:
D1. In embodiment D1, provided is a pharmaceutical composition comprising a compound of any one of the foregoing A, B, and C embodiments, or a pharmaceutically acceptable salt thereof, and a pharmaceutically acceptable excipient.
D2. In embodiment D2, provided is a method of degrading CDK2 in a cell via proteasome pathway which method comprises contacting the cell with a compound of any one of the foregoing A, B, and C embodiments, or a pharmaceutically acceptable salt thereof, or a pharmaceutical composition of embodiment D1.
D3. In embodiment D3, provided is a method of treating a disease mediated by CDK2 in a patient which method comprises administering to the patient in recognized need thereof, a therapeutically effective amount of a compound of any one of the foregoing A, B, and C embodiments or a pharmaceutical composition of embodiment D1.
D4. In embodiment D4, provided is a method of treating cancer in a patient which method comprises administering to the patient in need thereof, a therapeutically effective amount a compound of any one of the foregoing A, B, and C embodiments, or a pharmaceutically acceptable salt thereof, or a pharmaceutical composition of D1.
D5. In embodiment D5, the method of embodiment A194 is wherein the compound of any one of the foregoing A and B embodiments or a pharmaceutically acceptable salt thereof, or a pharmaceutical composition of D1 is administered in combination with at least one other anticancer agent.
D6. In embodiment D6, the method of embodiments D4 or D5 is wherein the cancer is lung cancer, skin cancer, bladder cancer, breast cancer, cervical cancer, colorectal cancer, cancer of the small intestine, colon cancer, rectal cancer, cancer of the anus, endometrial cancer, gastric cancer, head and neck cancer, liver cancer, ovarian cancer, prostate cancer, testicular cancer, uterine cancer, esophageal cancer, gall bladder cancer, pancreatic cancer, stomach cancer, thyroid cancer, or parathyroid cancer.

It is understood that the embodiments and subembodiments set forth above include all combination of embodiments and subembodiments listed therein.

Representative compounds of Formula (I) are shown in Compound Table 1 below:

TABLE 1

| | | | MS (ES, |
|---|---|---|---|
| | | | m/z): |
| Cpd No. | Structure | Name | [M + 1]+ |
| 1 | 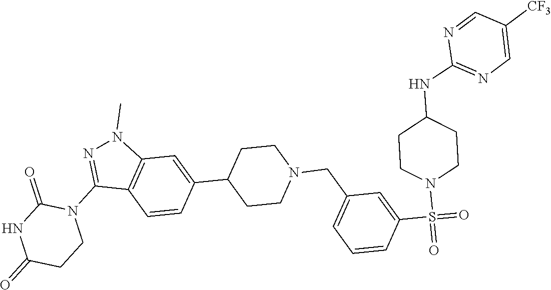 [SMILES: Cn1nc(N2CCC(=O)NC2=O)c2ccc(C3CCN(Cc4cccc(S(=O)(=O)N5CCC(Nc6ncc(C(F)(F)F)cn6)CC5)c4)CC3)cc21] | 1-(1-methyl-6-(1-(3-((4-((5- (trifluoromethyl)pyrimidin- 2-yl)amino)piperidin-1- yl)sulfonyl)benzyl)- piperidin-4-yl)-1H-indazol- 3-yl)dihydropyrimidine- 2,4(1H,3H)-dione | 726.1 |
| 2 | 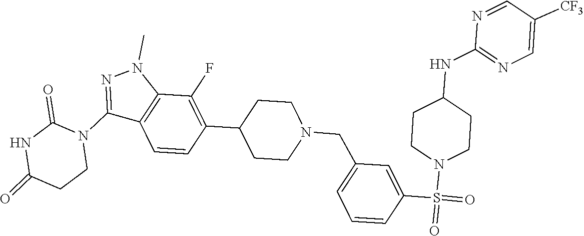 [SMILES: Cn1nc(N2CCC(=O)NC2=O)c2ccc(C3CCN(Cc4cccc(S(=O)(=O)N5CCC(Nc6ncc(C(F)(F)F)cn6)CC5)c4)CC3)c(F)c21] | 1-(7-fluoro-1-methyl-6-(1- (3-((4-((5- (trifluoromethyl)pyrimidin- 2-yl)amino)piperidin-1- yl)sulfonyl)benzyl)- piperidin-4-yl)-1H-indazol- 3-yl)dihydropyrimidine- 2,4(1H,3H)-dione | 744.0 |
| 3 | 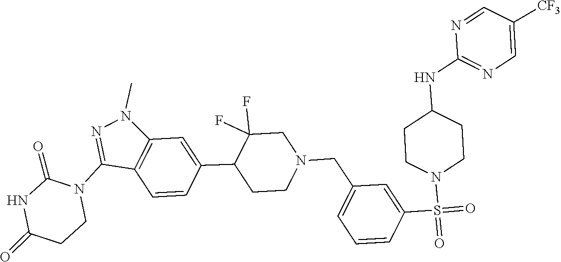 [SMILES: Cn1nc(N2CCC(=O)NC2=O)c2ccc(C3CCN(Cc4cccc(S(=O)(=O)N5CCC(Nc6ncc(C(F)(F)F)cn6)CC5)c4)CC3(F)F)cc21] | 1-(6-(3,3-difluoro-1-(3-((4- ((5- (trifluoromethyl)pyrimidin- 2-yl)amino)-piperidin-1- yl)sulfonyl)benzyl)- piperidin-4-yl)-1-methyl- 1H-indazol-3- yl)dihydropyrimidine- 2,4(1H,3H)-dione | 762.1 |
| 4 | 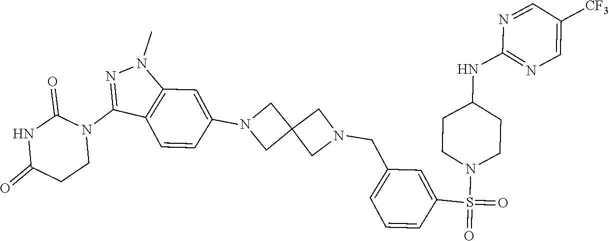 [SMILES: Cn1nc(N2CCC(=O)NC2=O)c2ccc(N3CC4(CN(Cc5cccc(S(=O)(=O)N6CCC(Nc7ncc(C(F)(F)F)cn7)CC6)c5)C4)C3)cc21] | 1-(1-methyl-6-(6-(3-((4-((5- (trifluoromethyl)pyrimidin- 2-yl)amino)piperidin-1- yl)sulfonyl)benzyl)-2,6- diazaspiro[3.3]heptan-2-yl)- 1H-indazol-3- yl)dihydropyrimidine- 2,4(1H,3H)-dione | 740.0 |
| 5 | 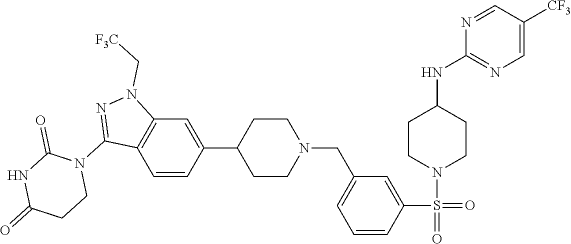 [SMILES: O=C1CCN(c2nn(CC(F)(F)F)c3cc(C4CCN(Cc5cccc(S(=O)(=O)N6CCC(Nc7ncc(C(F)(F)F)cn7)CC6)c5)CC4)ccc23)C(=O)N1] | 1-(1-(2,2,2-trifluoroethyl)- 6-(1-(3-((4-((5- (trifluoromethyl)pyrimidin- 2-yl)amino)-piperidin-1- yl)sulfonyl)benzyl)- piperidin-4-yl)-1H-indazol- 3-yl)dihydropyrimidine- 2,4(1H,3H)-dione | 794.0 |
| 6 | 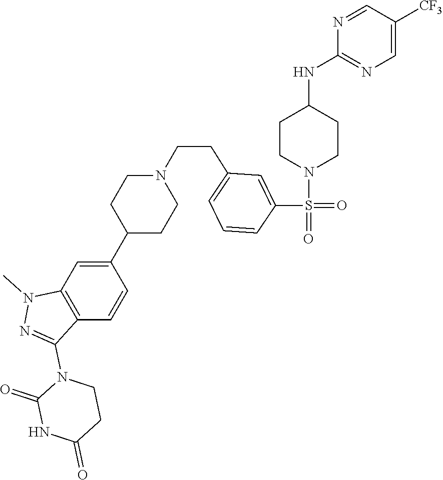 [SMILES: Cn1nc(N2CCC(=O)NC2=O)c2ccc(C3CCN(CCc4cccc(S(=O)(=O)N5CCC(Nc6ncc(C(F)(F)F)cn6)CC5)c4)CC3)cc21] | 1-(1-methyl-6-(1-(3-((4-((5- (trifluoromethyl)pyrimidin- 2-yl)amino)-piperidin-1- yl)sulfonyl)phenethyl) piperidin-4-yl)-1H- indazol-3- yl)dihydropyrimidine- 2,4(1H,3H)-dione | 740.3 |
| 7 | 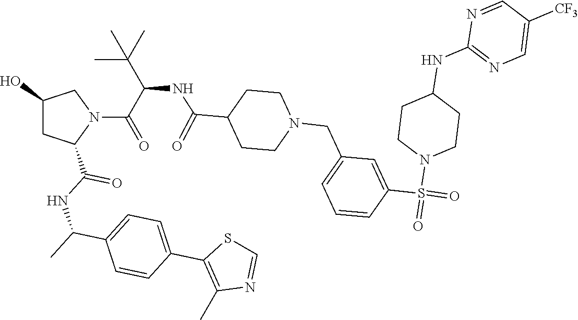 [SMILES: Cc1ncsc1-c1ccc([C@H](C)NC(=O)[C@@H]2C[C@@H](O)CN2C(=O)[C@H](NC(=O)C2CCN(Cc3cccc(S(=O)(=O)N4CCC(Nc5ncc(C(F)(F)F)cn5)CC4)c3)CC2)C(C)(C)C)cc1] | N-((R)-1-((2S,4R)-4- hydroxy-2-(((S)-1-(4-(4- methylthiazol-5- yl)phenyl)ethyl)- carbamoyl)pyrrolidin-1-yl)- 3,3-dimethyl-1-oxobutan-2- yl)-1-(3-((4-((5-(trifluoro- methyl)pyrimidin-2- yl)amino)piperidin-1- yl)sulfonyl)benzyl) piperidine-4-carboxamide | 954.3 |
| 8 | 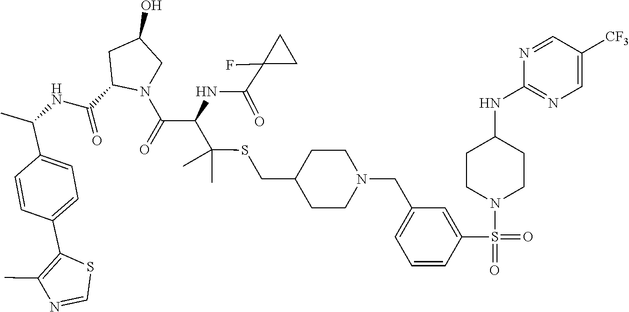 [SMILES: Cc1ncsc1-c1ccc([C@H](C)NC(=O)[C@@H]2C[C@@H](O)CN2C(=O)[C@@H](NC(=O)C2(F)CC2)C(C)(C)SCC2CCN(Cc3cccc(S(=O)(=O)N4CCC(Nc5ncc(C(F)(F)F)cn5)CC4)c3)CC2)cc1] | (2S,4R)-1-((R)-2-(1- fluorocyclopropane-1- carboxamido)-3-methyl-3- (((1-(3-((4-((5- (trifluoromethyl)pyrimidin- 2-yl)amino)-piperidin-1- yl)sulfonyl)benzyl) piperidin-4-yl)methyl) thio)butanoyl)- 4-hydroxy-N-((S)-1-(4-(4- methylthiazol-5-yl)phenyl)- ethyl)pyrrolidine-2- carboxamide | 1044.3 |
| 9 | 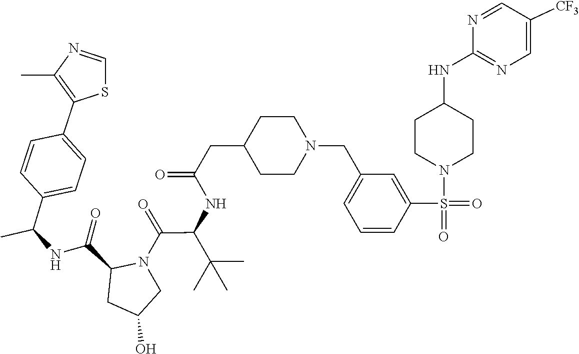 [SMILES: Cc1ncsc1-c1ccc([C@H](C)NC(=O)[C@@H]2C[C@@H](O)CN2C(=O)[C@@H](NC(=O)CC2CCN(Cc3cccc(S(=O)(=O)N4CCC(Nc5ncc(C(F)(F)F)cn5)CC4)c3)CC2)C(C)(C)C)cc1] | (2S,4R)-1-((S)-3,3- dimethyl-2-(2-(1-(3-((4-((5- (trifluoromethyl)pyrimidin- 2-yl)amino)piperidin-1- yl)sulfonyl)- benzyl)piperidin-4- yl)acetamido)butanoyl)-4- hydroxy-N-((S)-1-(4-(4- methylthiazol-5- yl)phenyl)ethyl)pyrrolidine- 2-carboxamide | 968.4 |
| 10 | 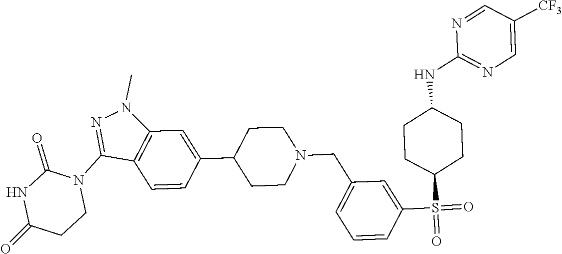 [SMILES: Cn1nc(N2CCC(=O)NC2=O)c2ccc(C3CCN(Cc4cccc(S(=O)(=O)[C@H]5CC[C@H](Nc6ncc(C(F)(F)F)cn6)CC5)c4)CC3)cc21] | 1-(1-methyl-6-(1-(3- (((1r,4r)-4-((5- (trifluoromethyl)pyrimidin- 2-yl)amino)- cyclohexyl)sulfonyl)benzyl) piperidin-4-yl)-1H-indazol- 3-yl)dihydropyrimidine- 2,4(1H,3H)-dione | 725.3 |
| 11 | 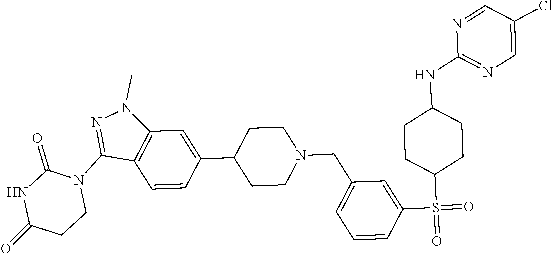 [SMILES: Cn1nc(N2CCC(=O)NC2=O)c2ccc(C3CCN(Cc4cccc(S(=O)(=O)C5CCC(Nc6ncc(Cl)cn6)CC5)c4)CC3)cc21] | 1-(6-(1-(3-((4-((5- chloropyrimidin-2- yl)amino)piperidin-1- yl)sulfonyl)benzyl)- piperidin-4-yl)-1-methyl- 1H-indazol-3- yl)dihydropyrimidine- 2,4(1H,3H)-dione | 692.0 |
| 12 | 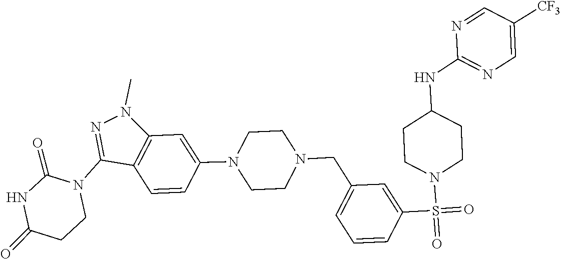 [SMILES: Cn1nc(N2CCC(=O)NC2=O)c2ccc(N3CCN(Cc4cccc(S(=O)(=O)N5CCC(Nc6ncc(C(F)(F)F)cn6)CC5)c4)CC3)cc21] | 1-(1-methyl-6-(4-(3-((4-((5- (trifluoro-methyl)pyrimidin- 2-yl)amino)piperidin-1- yl)sulfonyl)benzyl) piperazin-1-yl)-1H- indazol-3- yl)dihydropyrimidine- 2,4(1H,3H)-dione | 727.0 |
| 13 | 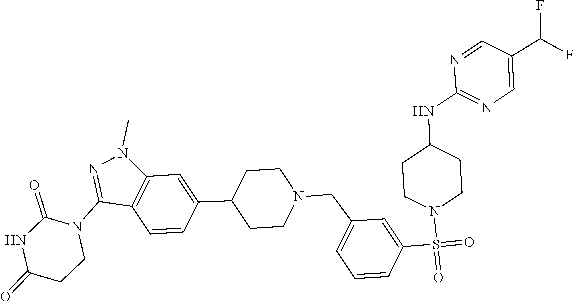 [SMILES: Cn1nc(N2CCC(=O)NC2=O)c2ccc(C3CCN(Cc4cccc(S(=O)(=O)N5CCC(Nc6ncc(C(F)F)cn6)CC5)c4)CC3)cc21] | 1-(6-(1-(3-((4-((5- (difluoromethyl)-pyrimidin- 2-yl)amino)piperidin-1- yl)sulfonyl)benzyl) piperidin-4-yl)-1-methyl- 1H-indazol- 3-yl)dihydropyrimidine- 2,4(1H,3H)-dione | 708.1 |
| 14 | 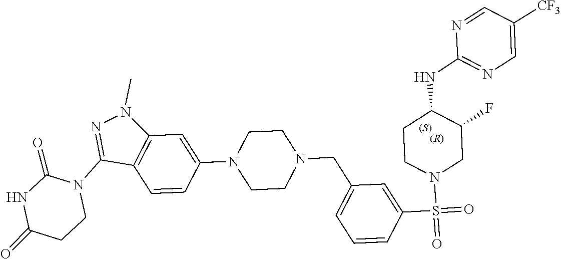 | 1-(6-(1-(3-(((3R,4S)-3- fluoro-4-((5- (trifluoromethyl)pyrimidin- 2-yl)amino)-piperidin-1- yl)sulfonyl)benzyl)- piperidin-4-yl)-1-methyl- 1H-indazol-3- yl)dihydropyrimidine- 2,4(1H,3H)-dione | 744.0 |
| 15 | 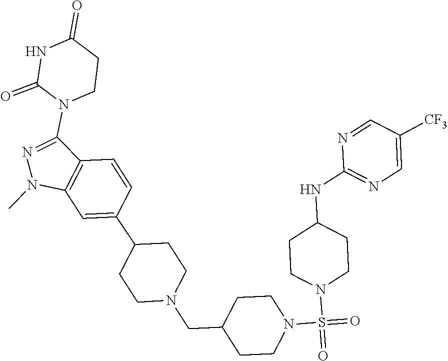 [SMILES: Cn1nc(N2CCC(=O)NC2=O)c2ccc(C3CCN(CC4CCN(S(=O)(=O)N5CCC(Nc6ncc(C(F)(F)F)cn6)CC5)CC4)CC3)cc21] | 1-(1-methyl-6-(1-((1-((4- ((5-(trifluoro- methyl)pyrimidin-2- yl)amino)piperidin-1- yl)sulfonyl)piperidin-4- yl)methyl)piperidin-4-yl)- 1H-indazol-3- yl)dihydropyrimidine- 2,4(1H,3H)-dione | 733.1 |
| 16 | 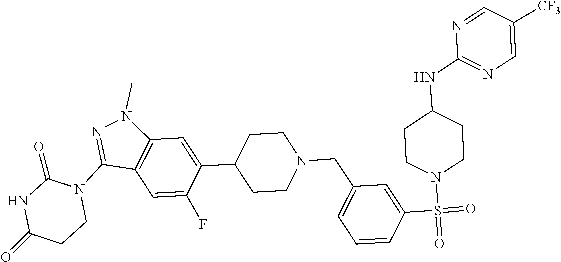 [SMILES: Cn1nc(N2CCC(=O)NC2=O)c2cc(F)c(C3CCN(Cc4cccc(S(=O)(=O)N5CCC(Nc6ncc(C(F)(F)F)cn6)CC5)c4)CC3)cc21] | 1-(5-fluoro-1-methyl-6-(1- (3-((4-((5- (trifluoromethyl)pyrimidin- 2-yl)amino)-piperidin-1- yl)sulfonyl)benzyl) piperidin-4-yl)- 1H-indazol-3- yl)dihydropyrimidine- 2,4(1H,3H)-dione | 744.1 |
| 17 | 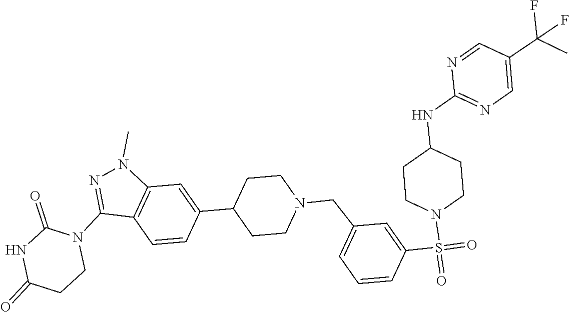 [SMILES: Cn1nc(N2CCC(=O)NC2=O)c2ccc(C3CCN(Cc4cccc(S(=O)(=O)N5CCC(Nc6ncc(C(C)(F)F)cn6)CC5)c4)CC3)cc21] | 1-(6-(1-(3-((4-((5-(1,1- difluoroethyl)-pyrimidin-2- yl)amino)piperidin-1- yl)sulfonyl)benzyl) piperidin-4-yl)-1-methyl- 1H-indazol- 3-yl)dihydropyrimidine- 2,4(1H,3H)-dione | 722.1 |
| 18 | 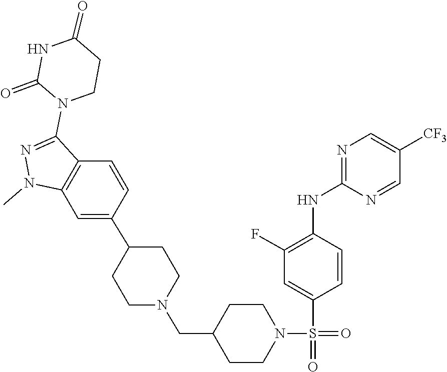 [SMILES: Cn1nc(N2CCC(=O)NC2=O)c2ccc(C3CCN(CC4CCN(S(=O)(=O)c5ccc(Nc6ncc(C(F)(F)F)cn6)c(F)c5)CC4)CC3)cc21] | 1-(6-(1-((1-((3-fluoro-4-((5- (trifluoro-methyl)pyrimidin- 2-yl)amino)phenyl)- sulfonyl)piperidin-4- yl)methyl)piperidin-4-yl)- 1-methyl-1H-indazol-3- yl)dihydropyrimidine- 2,4(1H,3H)-dione | 744.0 |
| 19 | 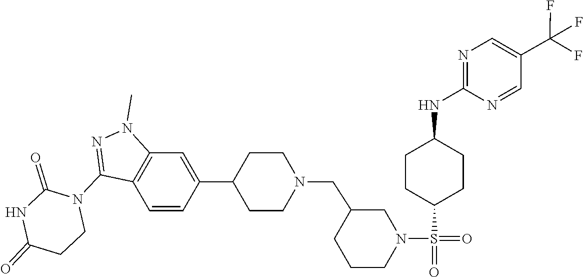 [SMILES: Cn1nc(N2CCC(=O)NC2=O)c2ccc(C3CCN(CC4CCCN(S(=O)(=O)[C@H]5CC[C@H](Nc6ncc(C(F)(F)F)cn6)CC5)C4)CC3)cc21] | 1-(1-methyl-6-(1-((1- (((1r,4r)-4-((5- (trifluoromethyl)pyrimidin- 2-yl)amino)- cyclohexyl)sulfonyl)piperidi n-3-yl)methyl)piperidin-4- yl)-1H-indazol-3- yl)dihydropyrimidine- 2,4(1H,3H)-dione | 732.3 |
| 20 | 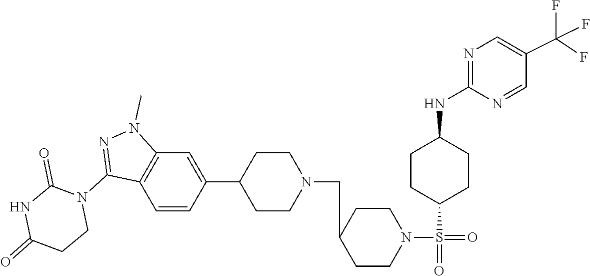 [SMILES: Cn1nc(N2CCC(=O)NC2=O)c2ccc(C3CCN(CC4CCN(S(=O)(=O)[C@H]5CC[C@H](Nc6ncc(C(F)(F)F)cn6)CC5)CC4)CC3)cc21] | 1-(1-methyl-6-(1-((1- (((1r,4r)-4-((5- (trifluoromethyl)pyrimidin- 2-yl)amino)- cyclohexyl)sulfonyl) piperidin-4-yl)methyl)- piperidin-4- yl)-1H-indazol-3-yl)- dihydropyrimidine- 2,4(1H,3H)-dione | 732.3 |
| 21 | 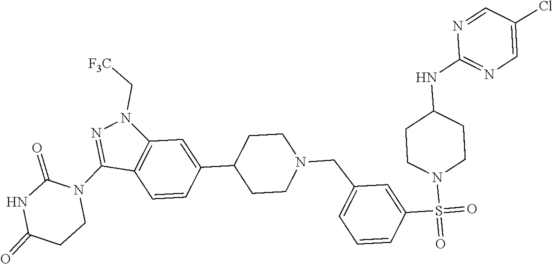 [SMILES: O=C1CCN(c2nn(CC(F)(F)F)c3cc(C4CCN(Cc5cccc(S(=O)(=O)N6CCC(Nc7ncc(Cl)cn7)CC6)c5)CC4)ccc23)C(=O)N1] | 1-(6-(1-(3-((4-((5- chloropyrimidin-2- yl)amino)piperidin-1- yl)sulfonyl)benzyl)- piperidin-4-yl)-1-(2,2,2- trifluoroethyl)-1H-indazol- 3-yl)dihydropyrimidine- 2,4(1H,3H)-dione | 760.0 |
| 22 | 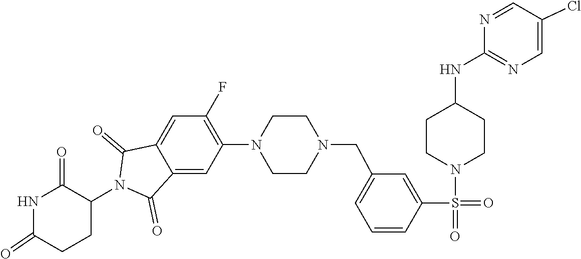 [SMILES: O=C1CCC(N2C(=O)c3cc(F)c(N4CCN(Cc5cccc(S(=O)(=O)N6CCC(Nc7ncc(Cl)cn7)CC6)c5)CC4)cc3C2=O)C(=O)N1] | 5-(4-(3-((4-((5- chloropyrimidin-2- yl)amino)piperidin-1- yl)sulfonyl)benzyl)- piperazin-1-yl)-2-(2,6- dioxopiperidin-3-yl)-6- fluoroisoindoline-1,3-dione | 725.0 |
| 23 | 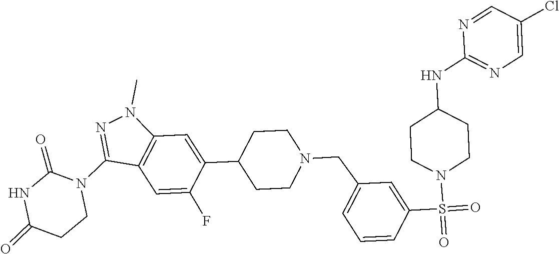 [SMILES: Cn1nc(N2CCC(=O)NC2=O)c2cc(F)c(C3CCN(Cc4cccc(S(=O)(=O)N5CCC(Nc6ncc(Cl)cn6)CC5)c4)CC3)cc21] | 1-(6-(1-(3-((4-((5- chloropyrimidin-2- yl)amino)piperidin-1- yl)sulfonyl)benzyl)- piperidin-4-yl)-5-fluoro-1- methyl-1H-indazol-3- yl)dihydropyrimidine- 2,4(1H,3H)-dione | 710.0 |
| 24 | 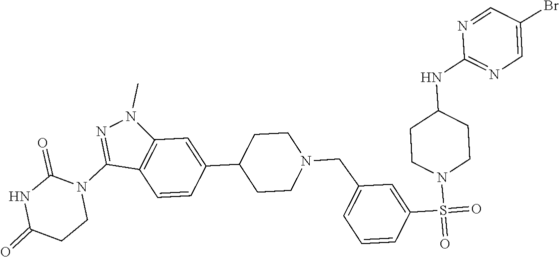 [SMILES: Cn1nc(N2CCC(=O)NC2=O)c2ccc(C3CCN(Cc4cccc(S(=O)(=O)N5CCC(Nc6ncc(Br)cn6)CC5)c4)CC3)cc21] | 1-(6-(1-(3-((4-((5- bromopyrimidin-2- yl)amino)piperidin-1- yl)sulfonyl)benzyl)- piperidin-4-yl)-1-methyl- 1H-indazol-3- yl)dihydropyrimidine- 2,4(1H,3H)-dione | 736.0 |
| 25 | 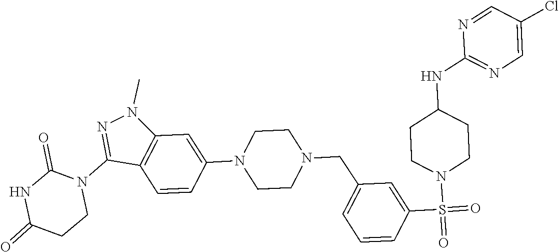 [SMILES: Cn1nc(N2CCC(=O)NC2=O)c2ccc(N3CCN(Cc4cccc(S(=O)(=O)N5CCC(Nc6ncc(Cl)cn6)CC5)c4)CC3)cc21] | 1-(6-(4-(3-((4-((5- chloropyrimidin-2- yl)amino)piperidin-1- yl)sulfonyl)benzyl)- piperazin-1-yl)-1-methyl- 1H-indazol-3- yl)dihydropyrimidine- 2,4(1H,3H)-dione | 693.0 |
| 26 | 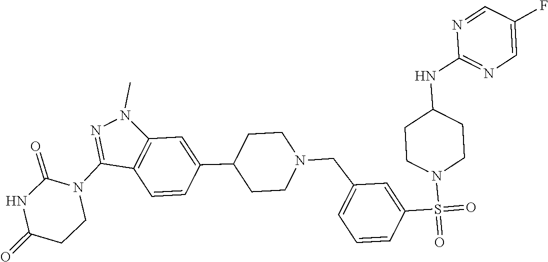 [SMILES: Cn1nc(N2CCC(=O)NC2=O)c2ccc(C3CCN(Cc4cccc(S(=O)(=O)N5CCC(Nc6ncc(F)cn6)CC5)c4)CC3)cc21] | 1-(6-(1-(3-((4-((5- fluoropyrimidin-2- yl)amino)piperidin-1- yl)sulfonyl)benzyl)- piperidin-4-yl)-1-methyl- 1H-indazol-3- yl)dihydropyrimidine- 2,4(1H,3H)-dione | 676.1 |
| 27 | 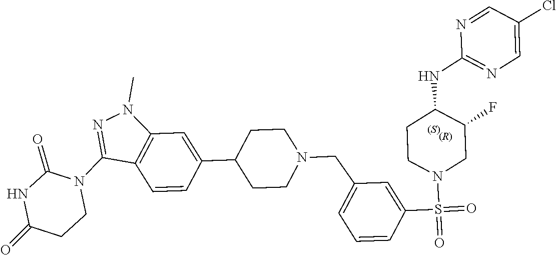 | 1-(6-(1-(3-(((3R,4S)-4-((5- chloropyrimidin-2- yl)amino)-3- fluoropiperidin-1- yl)sulfonyl)benzyl) piperidin-4-yl)-1-methyl- 1H-indazol- 3-yl)dihydropyrimidine- 2,4(1H,3H)-dione | 710.0 |
| 28 | 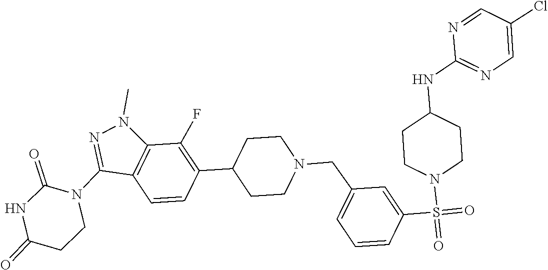 [SMILES: Cn1nc(N2CCC(=O)NC2=O)c2ccc(C3CCN(Cc4cccc(S(=O)(=O)N5CCC(Nc6ncc(Cl)cn6)CC5)c4)CC3)c(F)c21] | 1-(6-(1-(3-((4-((5- chloropyrimidin-2- yl)amino)piperidin-1- yl)sulfonyl)benzyl)- piperidin-4-yl)-7-fluoro- 1-methyl-1H-indazol-3- yl)dihydropyrimidine- 2,4(1H,3H)-dione | 710.0 |
| 29 | 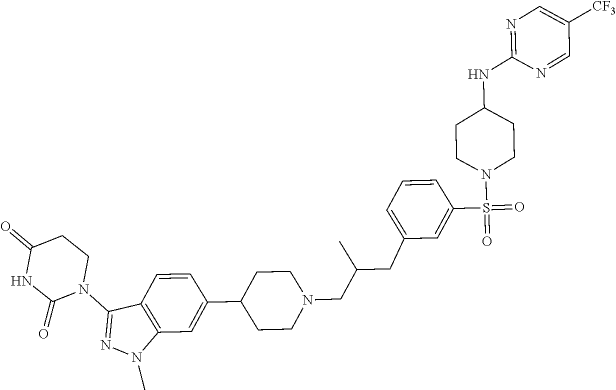 [SMILES: CC(Cc1cccc(S(=O)(=O)N2CCC(Nc3ncc(C(F)(F)F)cn3)CC2)c1)CN1CCC(c2ccc3c(N4CCC(=O)NC4=O)nn(C)c3c2)CC1] | 1-(1-methyl-6-(1-(2-methyl- 3-(3-((4-((5- (trifluoromethyl)pyrimidin- 2-yl)amino)piperidin-1- yl)sulfonyl)phenyl)- propyl)piperidin-4-yl)-1H- indazol-3- yl)dihydropyrimidine- 2,4(1H,3H)-dione | 768.3 |
| 30 | 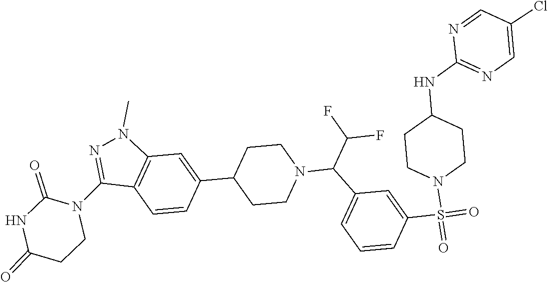 [SMILES: Cn1nc(N2CCC(=O)NC2=O)c2ccc(C3CCN(C(c4cccc(S(=O)(=O)N5CCC(Nc6ncc(Cl)cn6)CC5)c4)C(F)F)CC3)cc21] | 1-(6-(1-(1-(3-((4-((5- chloropyrimidin-2- yl)amino)piperidin-1- yl)sulfonyl)phenyl)-2,2- difluoroethyl)piperidin-4- yl)-1-methyl-1H-indazol-3- yl)dihydropyrimidine- 2,4(1H,3H)-dione | 742.1 |
| 32 | 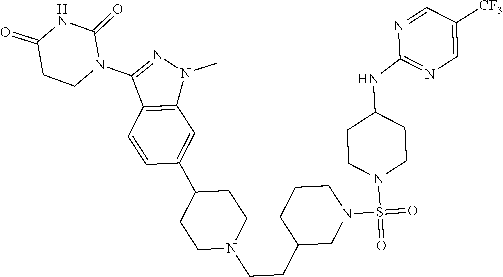 [SMILES: Cn1nc(N2CCC(=O)NC2=O)c2ccc(C3CCN(CCC4CCCN(S(=O)(=O)N5CCC(Nc6ncc(C(F)(F)F)cn6)CC5)C4)CC3)cc21] | 1-(1-methyl-6-(1-(2-(1-((4- ((5- (trifluoromethyl)pyrimidin- 2-yl)amino)-piperidin-1- yl)sulfonyl)piperidin-3- yl)ethyl)piperidin-4-yl)-1H- indazol-3- yl)dihydropyrimidine- 2,4(1H,3H)-dione | 747.6 |
| 33 | 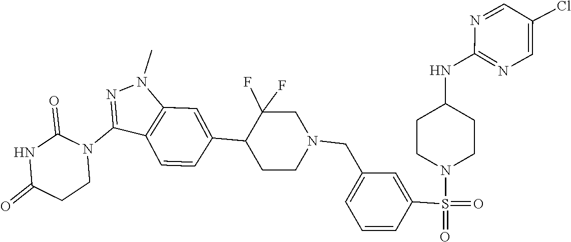 [SMILES: Cn1nc(N2CCC(=O)NC2=O)c2ccc(C3CCN(Cc4cccc(S(=O)(=O)N5CCC(Nc6ncc(Cl)cn6)CC5)c4)CC3(F)F)cc21] | 1-(6-(1-(3-((4-((5- chloropyrimidin-2- yl)amino)piperidin-1- yl)sulfonyl)benzyl)-3,3- difluoropiperidin-4-yl)-1- methyl-1H-indazol-3- yl)dihydropyrimidine- 2,4(1H,3H)-dione | 728.0 |
| 34 | 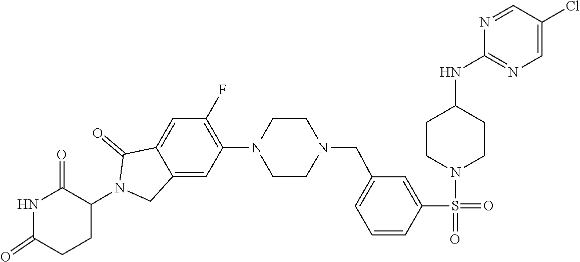 [SMILES: O=C1CCC(N2Cc3cc(N4CCN(Cc5cccc(S(=O)(=O)N6CCC(Nc7ncc(Cl)cn7)CC6)c5)CC4)c(F)cc3C2=O)C(=O)N1] | 3-(5-(4-(3-((4-((5- chloropyrimidin-2- yl)amino)piperidin-1- yl)sulfonyl)benzyl)- piperazin-1-yl)-6-fluoro-1- oxoisoindolin-2- yl)piperidine-2,6-dione | 711.0 |
| 35 | 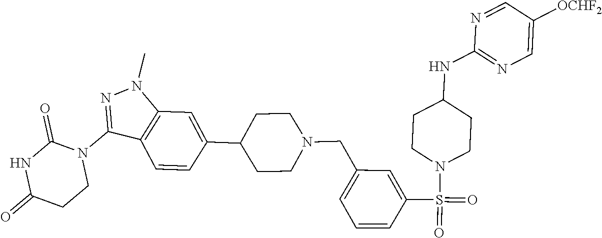 [SMILES: Cn1nc(N2CCC(=O)NC2=O)c2ccc(C3CCN(Cc4cccc(S(=O)(=O)N5CCC(Nc6ncc(OC(F)F)cn6)CC5)c4)CC3)cc21] | 1-(6-(1-(3-((4-((5- (difluoromethoxy)- pyrimidin-2- yl)amino)piperidin-1- yl)sulfonyl)benzyl) piperidin-4-yl)-1- methyl-1H-indazol- 3-yl)dihydropyrimidine- 2,4(1H,3H)-dione | 724.0 |
| 36 | 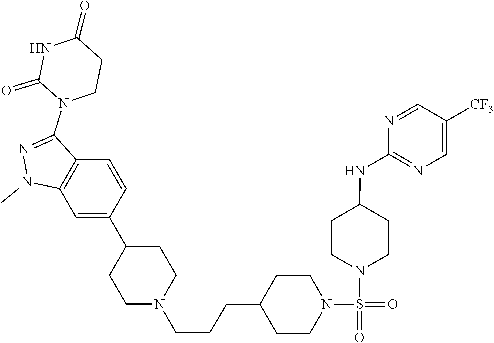 [SMILES: Cn1nc(N2CCC(=O)NC2=O)c2ccc(C3CCN(CCCC4CCN(S(=O)(=O)N5CCC(Nc6ncc(C(F)(F)F)cn6)CC5)CC4)CC3)cc21] | 1-(1-methyl-6-(1-(3-(1-((4- ((5-(trifluoro- methyl)pyrimidin-2- yl)amino)piperidin-1- yl)sulfonyl)piperidin-4- yl)propyl)piperidin-4-yl)- 1H-indazol-3- yl)dihydropyrimidine- 2,4(1H,3H)-dione | 761.1 |
| 38 | 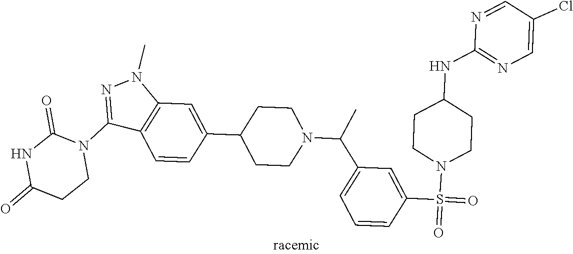 [SMILES: CC(c1cccc(S(=O)(=O)N2CCC(Nc3ncc(Cl)cn3)CC2)c1)N1CCC(c2ccc3c(N4CCC(=O)NC4=O)nn(C)c3c2)CC1] | 1-(6-(1-(1-(3-((4-((5- chloropyrimidin-2- yl)amino)piperidin-1- yl)sulfonyl)phenyl)- ethyl)piperidin-4-yl)-1- methyl-1H-indazol-3- yl)dihydropyrimidine- 2,4(1H,3H)-dione | 706.1 |
| 39 | 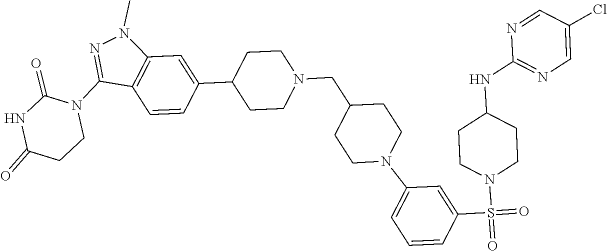 [SMILES: Cn1nc(N2CCC(=O)NC2=O)c2ccc(C3CCN(CC4CCN(c5cccc(S(=O)(=O)N6CCC(Nc7ncc(Cl)cn7)CC6)c5)CC4)CC3)cc21] | 1-(6-(1-((1-(3-((4-((5- chloropyrimidin-2- yl)amino)piperidin-1- yl)sulfonyl)phenyl)- piperidin-4- yl)methyl)piperidin-4-yl)-1- methyl-1H-indazol-3- yl)dihydropyrimidine- 2,4(1H,3H)-dione | 775.4 |
| 40 | 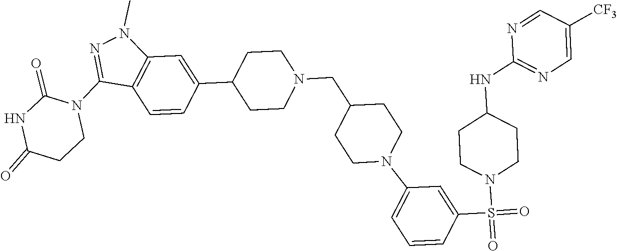 [SMILES: Cn1nc(N2CCC(=O)NC2=O)c2ccc(C3CCN(CC4CCN(c5cccc(S(=O)(=O)N6CCC(Nc7ncc(C(F)(F)F)cn7)CC6)c5)CC4)CC3)cc21] | 1-(1-methyl-6-(1-((1-(3-((4- ((5-(trifluoro- methyl)pyrimidin-2- yl)amino)piperidin-1- yl)sulfonyl)phenyl) piperidin-4-yl)methyl) piperidin-4- yl)-1H-indazol-3- yl)dihydropyrimidine- 2,4(1H,3H)-dione | 809.6 |
| 41 | 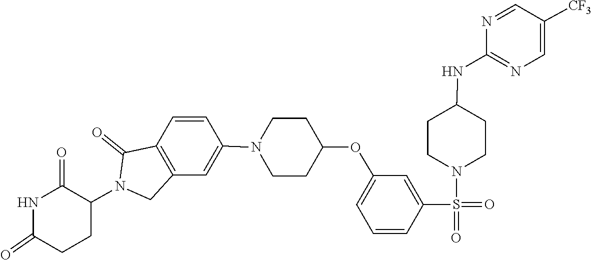 [SMILES: O=C1CCC(N2Cc3cc(N4CCC(Oc5cccc(S(=O)(=O)N6CCC(Nc7ncc(C(F)(F)F)cn7)CC6)c5)CC4)ccc3C2=O)C(=O)N1] | 3-(1-ox0-5-(4-(3-((4-((5- (trifluoromethyl)-pyrimidin- 2-yl)amino)piperidin-1- yl)sulfonyl)phenoxy) piperidin-1-yl)isoindolin- 2-yl)piperidine-2,6-dione | 728.5 |
| 42 | 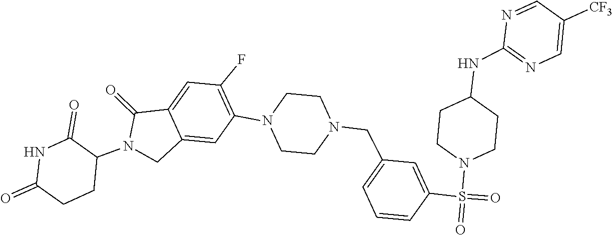 [SMILES: O=C1CCC(N2Cc3cc(N4CCN(Cc5cccc(S(=O)(=O)N6CCC(Nc7ncc(C(F)(F)F)cn7)CC6)c5)CC4)c(F)cc3C2=O)C(=O)N1] | 3-(6-fluoro-1-oxo-5-(4-(3- ((4-((5-(trifluoro- methyl)pyrimidin-2- yl)amino)piperidin-1- yl)sulfonyl)benzyl) piperazin-1-yl)isoindolin- 2-yl)piperidine-2,6-dione | 745.5 |
| 43 | 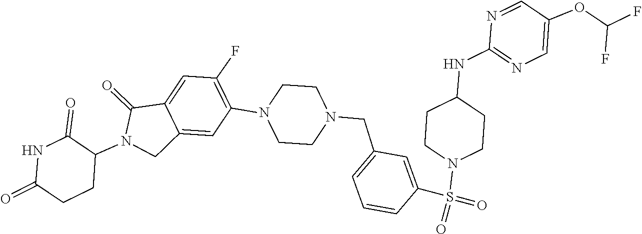 [SMILES: O=C1CCC(N2Cc3cc(N4CCN(Cc5cccc(S(=O)(=O)N6CCC(Nc7ncc(OC(F)F)cn7)CC6)c5)CC4)c(F)cc3C2=O)C(=O)N1] | 3-(5-(4-(3-((4-((5- (difluoromethoxy)- pyrimidin-2- yl)amino)piperidin-1- yl)sulfonyl)benzyl) piperazin-1-yl)-6- fluoro-1- oxoisoindolin-2- yl)piperidine-2,6-dione | 743.5 |
| 45 | 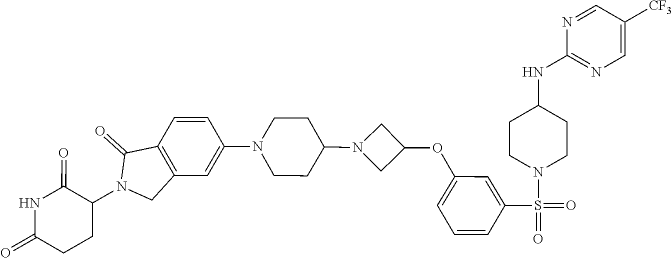 [SMILES: O=C1CCC(N2Cc3cc(N4CCC(N5CC(Oc6cccc(S(=O)(=O)N7CCC(Nc8ncc(C(F)(F)F)cn8)CC7)c6)C5)CC4)ccc3C2=O)C(=O)N1] | 3-(1-ox0-5-(4-(3-(3-((4-((5- (trifluoro-methyl)pyrimidin- 2-yl)amino)piperidin-1- yl)sulfonyl)phenoxy) azetidin-1-yl)piperidin-1- yl)isoindolin-2- yl)piperidine-2,6-dione | 783.5 |
| 46 | 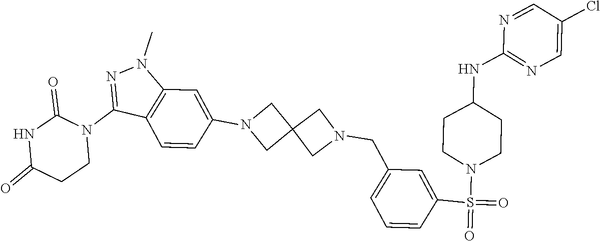 [SMILES: Cn1nc(N2CCC(=O)NC2=O)c2ccc(N3CC4(CN(Cc5cccc(S(=O)(=O)N6CCC(Nc7ncc(Cl)cn7)CC6)c5)C4)C3)cc21] | 1-(6-(6-(3-((4-((5- chloropyrimidin-2- yl)amino)piperidin-1- yl)sulfonyl)benzyl)-2,6- diazaspiro[3.3]heptan-2-yl)- 1-methyl-1H-indazol-3- yl)dihydropyrimidine- 2,4(1H,3H)-dione | 705.0 |
| 47 | 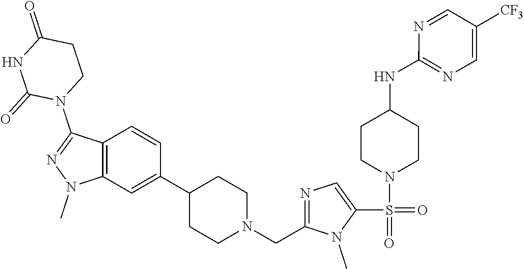 [SMILES: Cn1c(S(=O)(=O)N2CCC(Nc3ncc(C(F)(F)F)cn3)CC2)cnc1CN1CCC(c2ccc3c(N4CCC(=O)NC4=O)nn(C)c3c2)CC1] | 1-(1-methyl-6-(1-((1- methyl-5-((4-((5- (trifluoromethyl)pyrimidin- 2-yl)amino)-piperidin-1- yl)sulfonyl)-1H-imidazol-2- yl)methyl)piperidin-4-yl)- 1H-indazol-3- yl)dihydropyrimidine- 2,4(1H,3H)-dione | 730.1 |
| 48 | 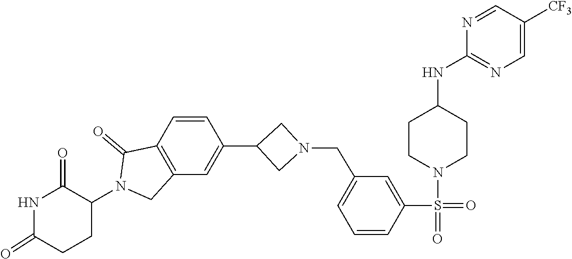 [SMILES: O=C1CCC(N2Cc3cc(C4CN(Cc5cccc(S(=O)(=O)N6CCC(Nc7ncc(C(F)(F)F)cn7)CC6)c5)C4)ccc3C2=O)C(=O)N1] | 3-(1-oxo-5-(1-(3-((4-((5- (trifluoromethyl)-pyrimidin- 2-yl)amino)piperidin-1- yl)sulfonyl)benzyl)azetidin- 3-yl)isoindolin-2- yl)piperidine-2,6-dione | 698.5 |
| 49 | 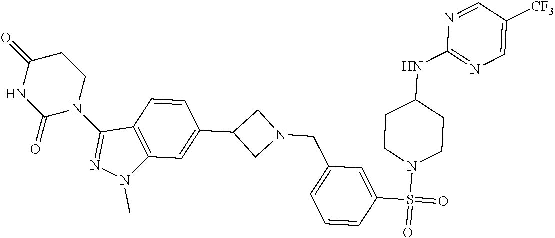 [SMILES: Cn1nc(N2CCC(=O)NC2=O)c2ccc(C3CN(Cc4cccc(S(=O)(=O)N5CCC(Nc6ncc(C(F)(F)F)cn6)CC5)c4)C3)cc21] | 1-(1-methyl-6-(1-(3-((4-((5- (trifluoro-methyl)pyrimidin- 2-yl)amino)piperidin-1- yl)sulfonyl)benzyl)azetidin- 3-yl)-1H-indazol-3- yl)dihydropyrimidine- 2,4(1H,3H)-dione | 698.0 |
| 50 | 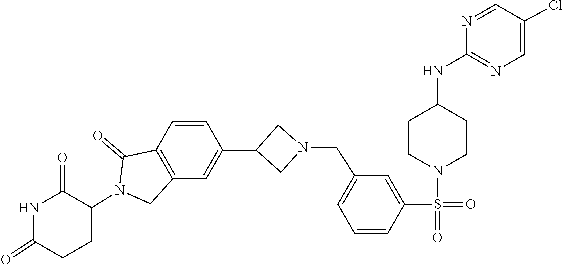 [SMILES: O=C1CCC(N2Cc3cc(C4CN(Cc5cccc(S(=O)(=O)N6CCC(Nc7ncc(Cl)cn7)CC6)c5)C4)ccc3C2=O)C(=O)N1] | 3-(5-(1-(3-((4-((5- chloropyrimidin-2- yl)amino)piperidin-1- yl)sulfonyl)benzyl)- azetidin-3-yl)-1- oxoisoindolin-2- yl)piperidine-2,6-dione | 664.3 |
| 51 | 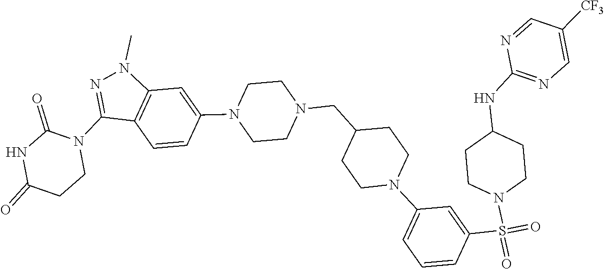 [SMILES: Cn1nc(N2CCC(=O)NC2=O)c2ccc(N3CCN(CC4CCN(c5cccc(S(=O)(=O)N6CCC(Nc7ncc(C(F)(F)F)cn7)CC6)c5)CC4)CC3)cc21] | 1-(1-methyl-6-(4-((1-(3-((4- ((5-(trifluoro- methyl)pyrimidin-2- yl)amino)piperidin-1- yl)sulfonyl)phenyl) piperidin-4-yl)methyl) piperazin-1- yl)-1H-indazol-3- yl)dihydropyrimidine- 2,4(1H,3H)-dione | 810.6 |
| 52 | 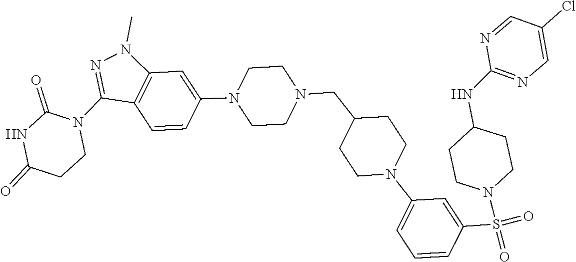 [SMILES: Cn1nc(N2CCC(=O)NC2=O)c2ccc(N3CCN(CC4CCN(c5cccc(S(=O)(=O)N6CCC(Nc7ncc(Cl)cn7)CC6)c5)CC4)CC3)cc21] | 1-(6-(4-((1-(3-((4-((5- chloropyrimidin-2- yl)amino)piperidin-1- yl)sulfonyl)phenyl)- piperidin-4- yl)methyl)piperazin-1-yl)- 1-methyl-1H-indazol-3- yl)dihydropyrimidine- 2,4(1H,3H)-dione | 776.4 |
| 53 | 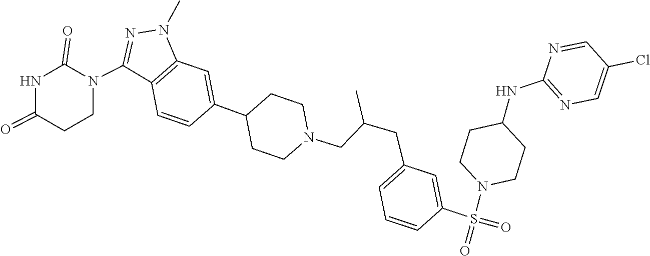 [SMILES: CC(Cc1cccc(S(=O)(=O)N2CCC(Nc3ncc(Cl)cn3)CC2)c1)CN1CCC(c2ccc3c(N4CCC(=O)NC4=O)nn(C)c3c2)CC1] | 1-(6-(1-(3-(3-((4-((5- chloropyrimidin-2- yl)amino)piperidin-1- yl)sulfonyl)phenyl)-2- methylpropyl)piperidin-4- yl)-1-methyl-1H-indazol-3- yl)dihydropyrimidine- 2,4(1H,3H)-dione | 734.3 |
| 54 | 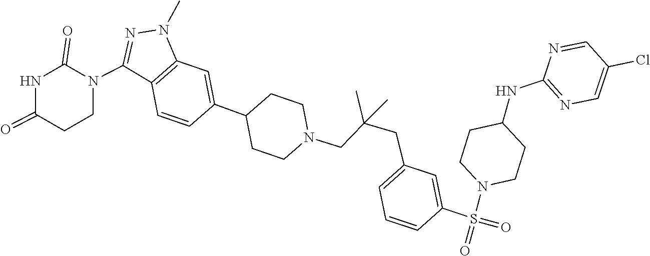 [SMILES: Cn1nc(N2CCC(=O)NC2=O)c2ccc(C3CCN(CC(C)(C)Cc4cccc(S(=O)(=O)N5CCC(Nc6ncc(Cl)cn6)CC5)c4)CC3)cc21] | 1-(6-(1-(3-(3-((4-((5- chloropyrimidin-2- yl)amino)piperidin-1- yl)sulfonyl)phenyl)-2,2- dimethylpropyl)piperidin-4- yl)-1-methyl-1H-indazol-3- yl)dihydropyrimidine- 2,4(1H,3H)-dione | 748.3 |
| 55 | 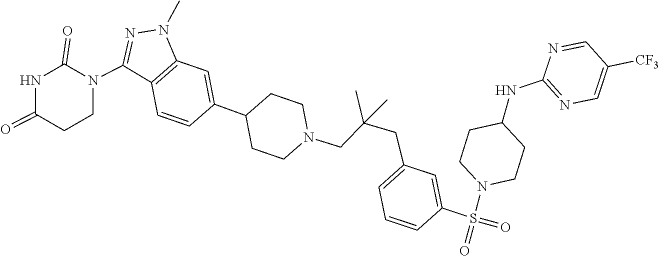 [SMILES: Cn1nc(N2CCC(=O)NC2=O)c2ccc(C3CCN(CC(C)(C)Cc4cccc(S(=O)(=O)N5CCC(Nc6ncc(C(F)(F)F)cn6)CC5)c4)CC3)cc21] | 1-(6-(1-(2,2-dimethyl-3-(3- ((4-((5-(trifluoro- methyl)pyrimidin-2- yl)amino)piperidin-1- yl)sulfonyl)phenyl)propyl) piperidin-4-yl)-1-methyl- 1H-indazol-3- yl)dihydropyrimidine- 2,4(1H,3H)-dione | 782.3 |
| 56 | 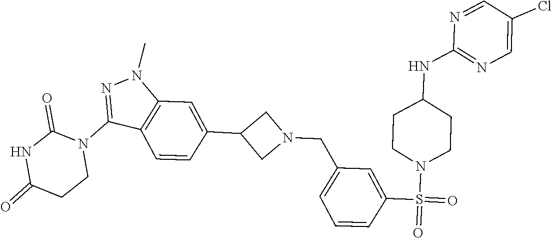 [SMILES: Cn1nc(N2CCC(=O)NC2=O)c2ccc(C3CN(Cc4cccc(S(=O)(=O)N5CCC(Nc6ncc(Cl)cn6)CC5)c4)C3)cc21] | 1-(6-(1-(3-((4-((5- chloropyrimidin-2- yl)amino)piperidin-1- yl)sulfonyl)benzyl)- azetidin-3-yl)-1-methyl-1H- indazol-3- yl)dihydropyrimidine- 2,4(1H,3H)-dione | 664.0 |
| 57 | 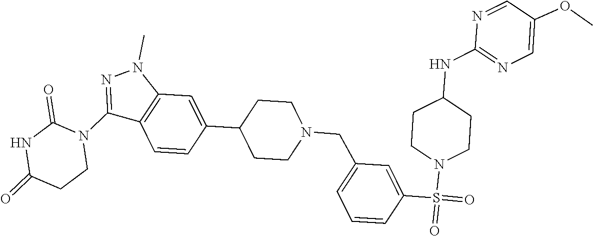 [SMILES: COc1cnc(NC2CCN(S(=O)(=O)c3cccc(CN4CCC(c5ccc6c(N7CCC(=O)NC7=O)nn(C)c6c5)CC4)c3)CC2)nc1] | 1-(6-(1-(3-((4-((5- methoxypyrimidin-2- yl)amino)piperidin-1- yl)sulfonyl)benzyl)- piperidin-4-yl)-1-methyl- 1H-indazol-3- yl)dihydropyrimidine- 2,4(1H,3H)-dione | 688.1 |
| 58 | 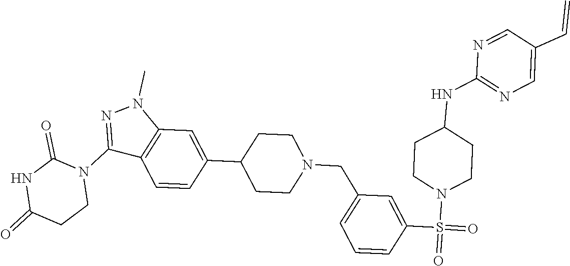 [SMILES: C=Cc1cnc(NC2CCN(S(=O)(=O)c3cccc(CN4CCC(c5ccc6c(N7CCC(=O)NC7=O)nn(C)c6c5)CC4)c3)CC2)nc1] | 1-(1-methyl-6-(1-(3-((4-((5- vinylpyrimidin-2- yl)amino)piperidin-1- yl)sulfonyl)benzyl)- piperidin-4-yl)-1H-indazol- 3-yl)dihydropyrimidine- 2,4(1H,3H)-dione | 684.1 |
| 59 | 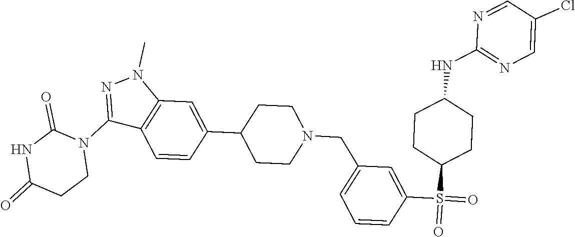 [SMILES: Cn1nc(N2CCC(=O)NC2=O)c2ccc(C3CCN(Cc4cccc(S(=O)(=O)[C@H]5CC[C@H](Nc6ncc(Cl)cn6)CC5)c4)CC3)cc21] | 1-(6-(1-(3-(((1r,4r)-4-((5- chloropyrimidin-2- yl)amino)cyclohexyl) sulfonyl)benzyl) piperidin-4-yl)-1- methyl-1H-indazol-3- yl)dihydropyrimidine- 2,4(1H,3H)-dione | 691.3 |
| 60 | 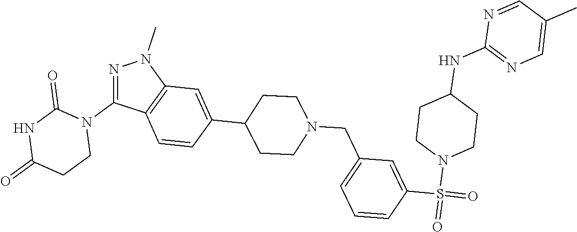 [SMILES: Cc1cnc(NC2CCN(S(=O)(=O)c3cccc(CN4CCC(c5ccc6c(N7CCC(=O)NC7=O)nn(C)c6c5)CC4)c3)CC2)nc1] | 1-(1-methyl-6-(1-(3-((4-((5- methyl-pyrimidin-2- yl)amino)piperidin-1- yl)sulfonyl)benzyl) piperidin-4-yl)-1H- indazol-3- yl)dihydropyrimidine- 2,4(1H,3H)-dione | 672.1 |
| 61 | 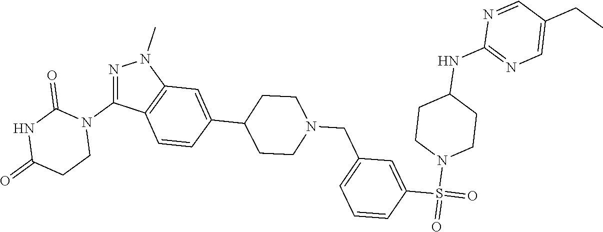 [SMILES: CCc1cnc(NC2CCN(S(=O)(=O)c3cccc(CN4CCC(c5ccc6c(N7CCC(=O)NC7=O)nn(C)c6c5)CC4)c3)CC2)nc1] | 1-(6-(1-(3-((4-((5- ethylpyrimidin-2- yl)amino)piperidin-1- yl)sulfonyl)benzyl)- piperidin-4-yl)-1-methyl- 1H-indazol-3- yl)dihydropyrimidine- 2,4(1H,3H)-dione | 686.1 |
| 62 | 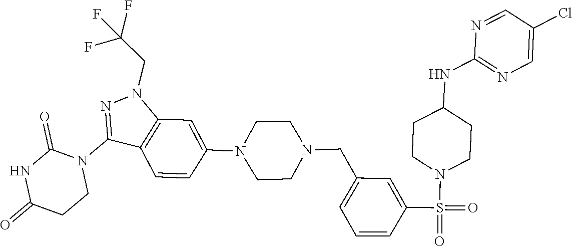 [SMILES: O=C1CCN(c2nn(CC(F)(F)F)c3cc(N4CCN(Cc5cccc(S(=O)(=O)N6CCC(Nc7ncc(Cl)cn7)CC6)c5)CC4)ccc23)C(=O)N1] | 1-(6-(4-(3-((4-((5- chloropyrimidin-2- yl)amino)piperidin-1- yl)sulfonyl)benzyl)- piperazin-1-yl)-1-(2,2,2- trifluoroethyl)-1H-indazol- 3-yl)dihydropyrimidine- 2,4(1H,3H)-dione | 761.3 |
| 63 | 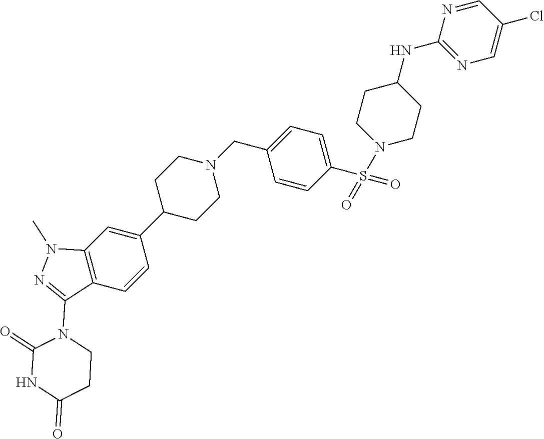 [SMILES: Cn1nc(N2CCC(=O)NC2=O)c2ccc(C3CCN(Cc4ccc(S(=O)(=O)N5CCC(Nc6ncc(Cl)cn6)CC5)cc4)CC3)cc21] | 1-(6-(1-(4-((4-((5- chloropyrimidin-2- yl)amino)piperidin-1- yl)sulfonyl)benzyl)- piperidin-4-yl)-1-methyl- 1H-indazol-3- yl)dihydropyrimidine- 2,4(1H,3H)-dione | 692.1 |
| 64 | 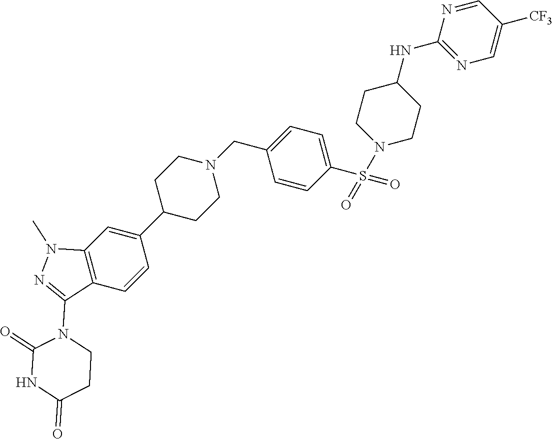 [SMILES: Cn1nc(N2CCC(=O)NC2=O)c2ccc(C3CCN(Cc4ccc(S(=O)(=O)N5CCC(Nc6ncc(C(F)(F)F)cn6)CC5)cc4)CC3)cc21] | 1-(1-methyl-6-(1-(4-((4-((5- (trifluoro-methyl)pyrimidin- 2-yl)amino)piperidin-1- yl)sulfonyl)benzyl) piperidin-4-yl)-1H- indazol-3- yl)dihydropyrimidine- 2,4(1H,3H)-dione | 726.0 |
| 65 | 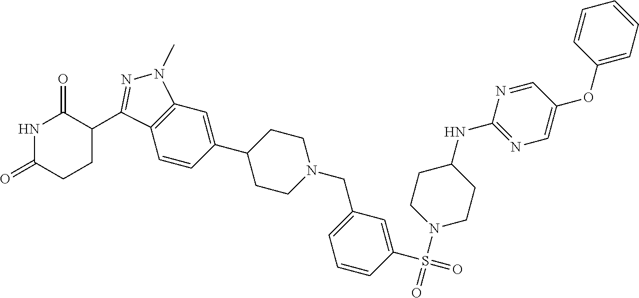 [SMILES: Cn1nc(C2CCC(=O)NC2=O)c2ccc(C3CCN(Cc4cccc(S(=O)(=O)N5CCC(Nc6ncc(Oc7ccccc7)cn6)CC5)c4)CC3)cc21] | 1-(1-methyl-6-(1-(3-((4-((5- phenoxy-pyrimidin-2- yl)amino)piperidin-1- yl)sulfonyl)benzyl) piperidin-4-yl)-1H- indazol-3- yl)dihydropyrimidine- 2,4(1H,3H)-dione | 750.1 |
| 66 | 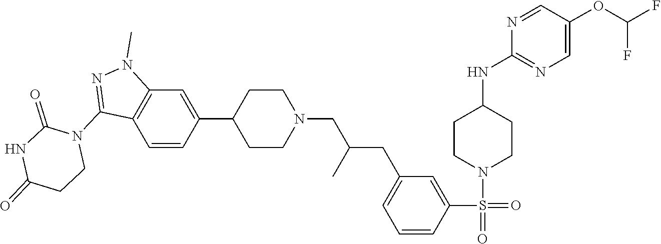 [SMILES: CC(Cc1cccc(S(=O)(=O)N2CCC(Nc3ncc(OC(F)F)cn3)CC2)c1)CN1CCC(c2ccc3c(N4CCC(=O)NC4=O)nn(C)c3c2)CC1] | 1-(6-(1-(3-(3-((4-((5- (difluoromethoxy)- pyrimidin-2- yl)amino)piperidin-1- yl)sulfonyl)phenyl)-2- methylpropyl)-piperidin-4- yl)-1-methyl-1H-indazol-3- yl)dihydropyrimidine- 2,4(1H,3H)-dione | 766.6 |
| 67 | 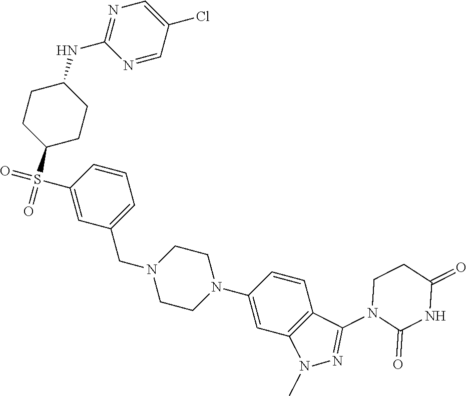 [SMILES: Cn1nc(N2CCC(=O)NC2=O)c2ccc(N3CCN(Cc4cccc(S(=O)(=O)[C@H]5CC[C@H](Nc6ncc(Cl)cn6)CC5)c4)CC3)cc21] | 1-(6-(4-(3-(((1r,4r)-4-((5- chloropyrimidin-2- yl)amino)cyclohexyl)sulfon yl)benzyl)piperazin-1-yl)-1- methyl-1H-indazol-3- yl)dihydropyrimidine- 2,4(1H,3H)-dione | 692.2 |
| 69 | 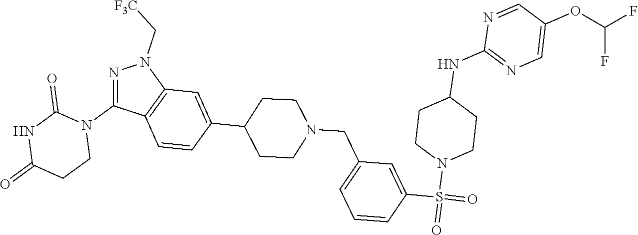 [SMILES: O=C1CCN(c2nn(CC(F)(F)F)c3cc(C4CCN(Cc5cccc(S(=O)(=O)N6CCC(Nc7ncc(OC(F)F)cn7)CC6)c5)CC4)ccc23)C(=O)N1] | 1-(6-(1-(3-((4-((5- (difluoromethoxy)- pyrimidin-2- yl)amino)piperidin-1- yl)sulfonyl)benzyl) piperidin-4-yl)-1-(2,2,2- trifluoroethyl)-1H-indazol- 3-yl)dihydro-pyrimidine- 2,4(1H,3H)-dione | 792.0 |
| 70 | 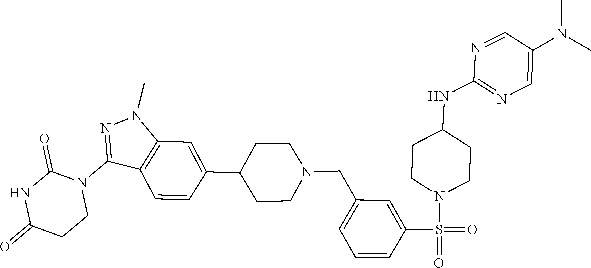 [SMILES: CN(C)c1cnc(NC2CCN(S(=O)(=O)c3cccc(CN4CCC(c5ccc6c(N7CCC(=O)NC7=O)nn(C)c6c5)CC4)c3)CC2)nc1] | 1-(6-(1-(3-((4-((5- (dimethylamino)-pyrimidin- 2-yl)amino)piperidin-1-yl)- sulfonyl)benzyl)piperidin-4- yl)-1-methyl-1H-indazol-3- yl)dihydropyrimidine- 2,4(1H,3H)-dione | 701.4 |
| 71 | 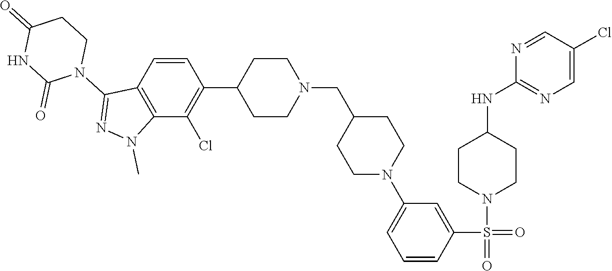 [SMILES: Cn1nc(N2CCC(=O)NC2=O)c2ccc(C3CCN(CC4CCN(c5cccc(S(=O)(=O)N6CCC(Nc7ncc(Cl)cn7)CC6)c5)CC4)CC3)c(Cl)c21] | 1-(7-chloro-6-(1-((1-(3-((4- ((5-chloro-pyrimidin-2- yl)amino)piperidin-1- yl)sulfonyl)phenyl) piperidin-4-yl)methyl)- piperidin-4-yl)-1-methyl- 1H-indazol-3- yl)dihydropyrimidine- 2,4(1H,3H)-dione | 809.1 |
| 72 | 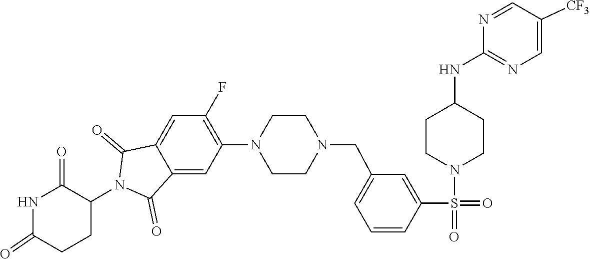 [SMILES: O=C1CCC(N2C(=O)c3cc(F)c(N4CCN(Cc5cccc(S(=O)(=O)N6CCC(Nc7ncc(C(F)(F)F)cn7)CC6)c5)CC4)cc3C2=O)C(=O)N1] | 2-(2,6-dioxopiperidin-3-yl)- 5-fluoro-6-(4-(3-((4-((5- (trifluoromethyl)pyrimidin- 2-yl)amino)piperidin-1- yl)sulfonyl)benzyl)- piperazin-1-yl)isoindoline- 1,3-dione | 759.0 |
| 73 | 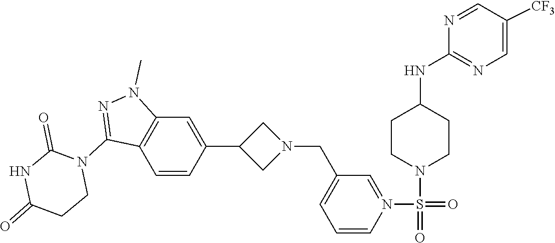 | 1-(1-methyl-6-(1-(3-((4-((5- (trifluoro-methyl)pyrimidin- 2-yl)amino)piperidin-1- yl)sulfonyl)benzyl)azetidin- 3-yl)-1H-indazol-3- yl)dihydropyrimidine- 2,4(1H,3H)-dione | 698.0 |
| 74 | 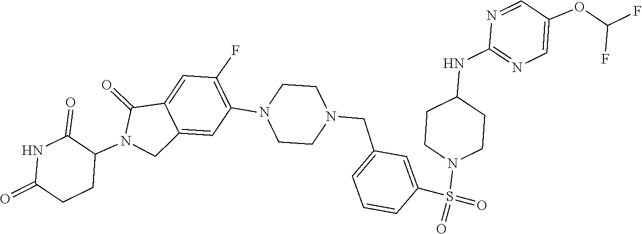 [SMILES: O=C1CCC(N2Cc3cc(N4CCN(Cc5cccc(S(=O)(=O)N6CCC(Nc7ncc(OC(F)F)cn7)CC6)c5)CC4)c(F)cc3C2=O)C(=O)N1] | 3-(5-(4-(3-((4-((5- (difluoromethoxy)- pyrimidin-2- yl)amino)piperidin-1- yl)sulfonyl)benzyl) piperazin-1-yl)-6- fluoro-1- oxoisoindolin-2- yl)piperidine-2,6-dione | 743.1 |
| 75 | 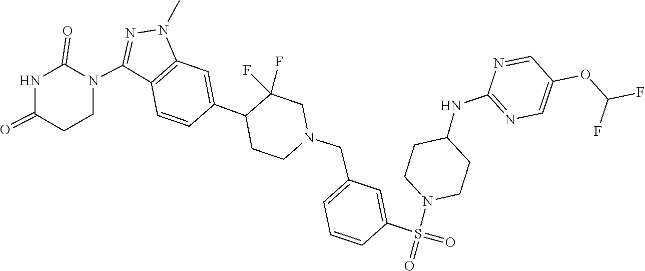 [SMILES: Cn1nc(N2CCC(=O)NC2=O)c2ccc(C3CCN(Cc4cccc(S(=O)(=O)N5CCC(Nc6ncc(OC(F)F)cn6)CC5)c4)CC3(F)F)cc21] | 1-(6-(1-(3-((4-((5- (difluoromethoxy)- pyrimidin-2- yl)amino)piperidin-1- yl)sulfonyl)benzyl)-3,3- difluoropiperidin-4-yl)-1- methyl-1H-indazol-3- yl)dihydropyrimidine- 2,4(1H,3H)-dione | 760.4 |
| 76 | 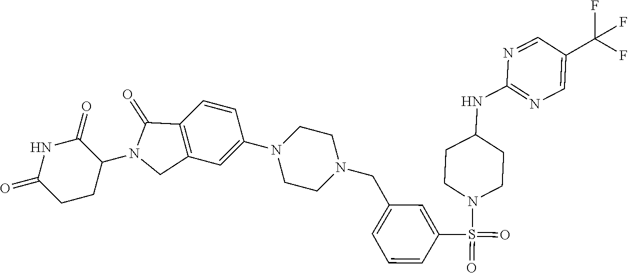 [SMILES: O=C1CCC(N2Cc3cc(N4CCN(Cc5cccc(S(=O)(=O)N6CCC(Nc7ncc(C(F)(F)F)cn7)CC6)c5)CC4)ccc3C2=O)C(=O)N1] | 3-(1-oxo-5-(4-(3-((4-((5- (trifluoromethyl)-pyrimidin- 2-yl)amino)piperidin-1- yl)sulfonyl)benzyl) piperazin-1-yl) isoindolin-2- yl)piperidine-2,6-dione | 727.0 |
| 77 | 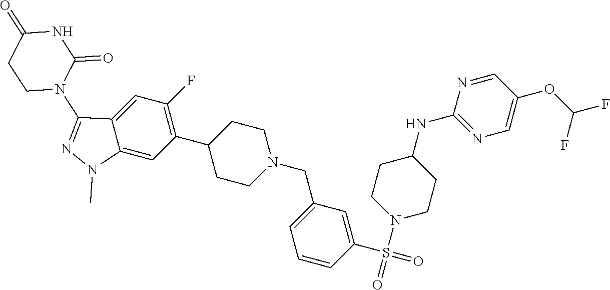 [SMILES: Cn1nc(N2CCC(=O)NC2=O)c2cc(F)c(C3CCN(Cc4cccc(S(=O)(=O)N5CCC(Nc6ncc(OC(F)F)cn6)CC5)c4)CC3)cc21] | 1-(6-(1-(3-((4-((5- (difluoromethoxy)- pyrimidin-2- yl)amino)piperidin-1- yl)sulfonyl)benzyl) piperidin-4-yl)-5- fluoro-1-methyl- 1H-indazol-3- yl)dihydropyrimidine- 2,4(1H,3H)-dione | 742.1 |
| 78 | 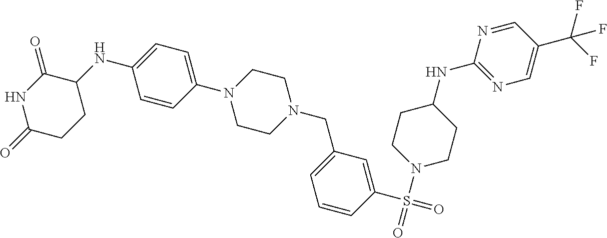 [SMILES: O=C1CCC(Nc2ccc(N3CCN(Cc4cccc(S(=O)(=O)N5CCC(Nc6ncc(C(F)(F)F)cn6)CC5)c4)CC3)cc2)C(=O)N1] | 3-((4-(4-(3-((4-((5- (trifluoromethyl)-pyrimidin- 2-yl)amino)piperidin-1- yl)sulfonyl)benzyl) piperazin-1- yl)phenyl)amino) piperidine-2,6-dione | 686.9 |
| 79 | 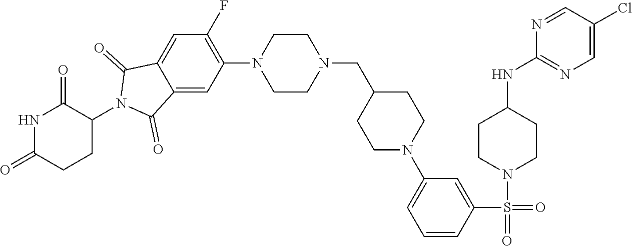 [SMILES: O=C1CCC(N2C(=O)c3cc(F)c(N4CCN(CC5CCN(c6cccc(S(=O)(=O)N7CCC(Nc8ncc(Cl)cn8)CC7)c6)CC5)CC4)cc3C2=O)C(=O)N1] | 5-(4-((1-(3-((4-((5- chloropyrimidin-2- yl)amino)piperidin-1- yl)sulfonyl)phenyl)- piperidin-4- yl)methyl)piperazin-1-yl)-2- (2,6-dioxopiperidin-3-yl)-6- fluoroisoindoline-1,3-dione | 808.3 |
| 80 | 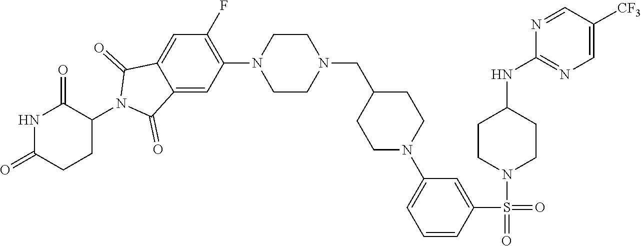 [SMILES: O=C1CCC(N2C(=O)c3cc(F)c(N4CCN(CC5CCN(c6cccc(S(=O)(=O)N7CCC(Nc8ncc(C(F)(F)F)cn8)CC7)c6)CC5)CC4)cc3C2=O)C(=O)N1] | 2-(2,6-dioxopiperidin-3-yl)- 5-fluoro-6-(4-((1-(3-((4-((5- (trifluoromethyl)pyrimidin- 2-yl)amino)piperidin-1- yl)sulfonyl)phenyl)- piperidin-4- yl)methyl)piperazin-1- yl)isoindoline-1,3-dione | 842.4 |
| 81 | 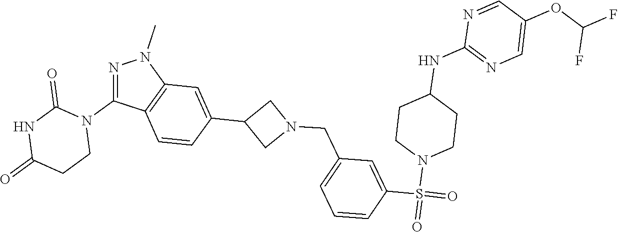 [SMILES: Cn1nc(N2CCC(=O)NC2=O)c2ccc(C3CN(Cc4cccc(S(=O)(=O)N5CCC(Nc6ncc(OC(F)F)cn6)CC5)c4)C3)cc21] | 1-(6-(1-(3-((4-((5- (difluoromethoxy)- pyrimidin-2- yl)amino)piperidin-1- yl)sulfonyl)benzyl)azetidin- 3-yl)-1-methyl-1H-indazol- 3-yl)dihydropyrimidine- 2,4(1H,3H)-dione | 696.0 |
| 82 | 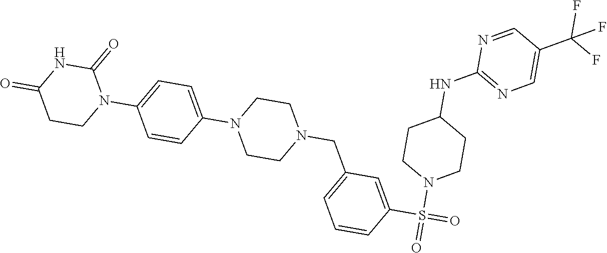 [SMILES: O=C1CCN(c2ccc(N3CCN(Cc4cccc(S(=O)(=O)N5CCC(Nc6ncc(C(F)(F)F)cn6)CC5)c4)CC3)cc2)C(=O)N1] | 1-(4-(4-(3-((4-((5- (trifluoromethyl)-pyrimidin- 2-yl)amino)piperidin-1- yl)sulfonyl)benzyl) piperazin-1-yl)phenyl) dihydropyrimidine- 2,4(1H,3H)-dione | 673.0 |
| 83 | 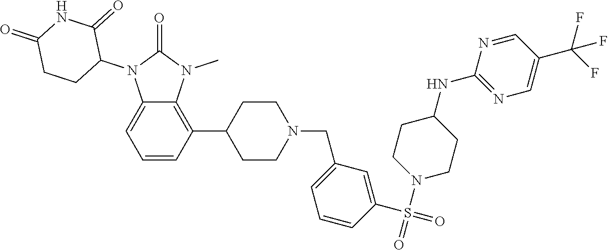 [SMILES: Cn1c(=O)n(C2CCC(=O)NC2=O)c2cccc(C3CCN(Cc4cccc(S(=O)(=O)N5CCC(Nc6ncc(C(F)(F)F)cn6)CC5)c4)CC3)c21] | 3-(3-methyl-2-oxo-4-(1-(3- ((4-((5- (trifluoromethyl)pyrimidin- 2-yl)amino)piperidin-1- yl)sulfonyl)benzyl)- piperidin-4-yl)-2,3-dihydro- 1H-benzo[d]imidazol-1- yl)piperidine-2,6-dione | 741.5 |
| 84 | 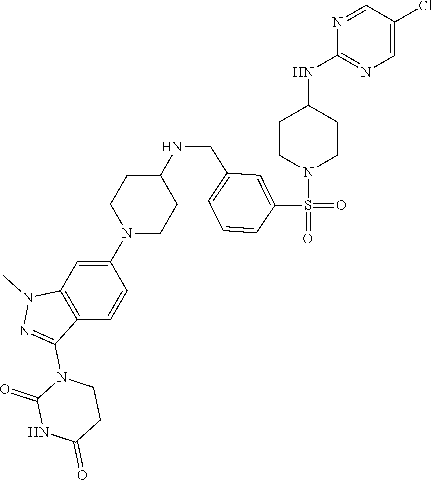 [SMILES: Cn1nc(N2CCC(=O)NC2=O)c2ccc(N3CCC(NCc4cccc(S(=O)(=O)N5CCC(Nc6ncc(Cl)cn6)CC5)c4)CC3)cc21] | 1-(6-(4-((3-((4-((5- chloropyrimidin-2- yl)amino)piperidin-1- yl)sulfonyl)benzyl)- amino)piperidin-1-yl)-1- methyl-1H-indazol-3- yl)dihydropyrimidine- 2,4(1H,3H)-dione | 707.1 |
| 85 | 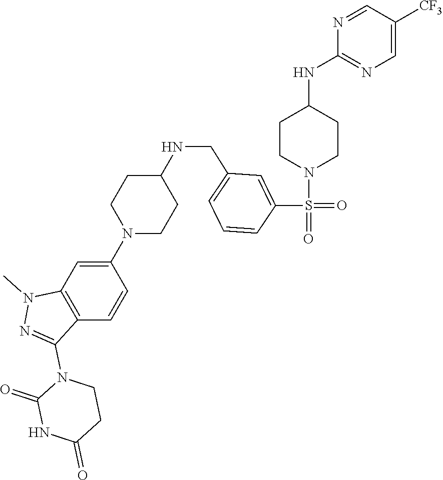 [SMILES: Cn1nc(N2CCC(=O)NC2=O)c2ccc(N3CCC(NCc4cccc(S(=O)(=O)N5CCC(Nc6ncc(C(F)(F)F)cn6)CC5)c4)CC3)cc21] | 1-(1-methyl-6-(4-((3-((4- ((5-(trifluoro- methyl)pyrimidin-2- yl)amino)piperidin-1- yl)sulfonyl)benzyl)amino) piperidin-1-yl)- 1H-indazol-3- yl)dihydropyrimidine- 2,4(1H,3H)-dione | 741.1 |
| 86 | 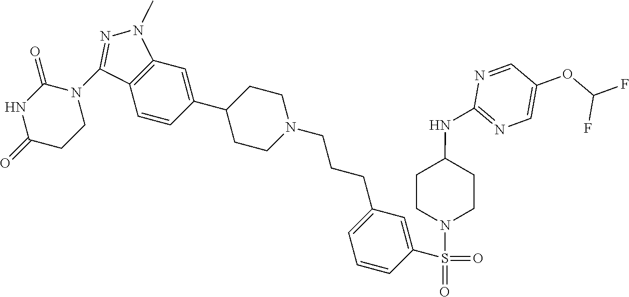 [SMILES: Cn1nc(N2CCC(=O)NC2=O)c2ccc(C3CCN(CCCc4cccc(S(=O)(=O)N5CCC(Nc6ncc(OC(F)F)cn6)CC5)c4)CC3)cc21] | 1-(6-(1-(3-(3-((4-((5- (difluoromethoxy)- pyrimidin-2- yl)amino)piperidin-1- yl)sulfonyl)phenyl)propyl) piperidin-4-yl)-1-methyl- 1H-indazol-3- yl)dihydropyrimidine- 2,4(1H,3H)-dione | 752.1 |
| 87 | 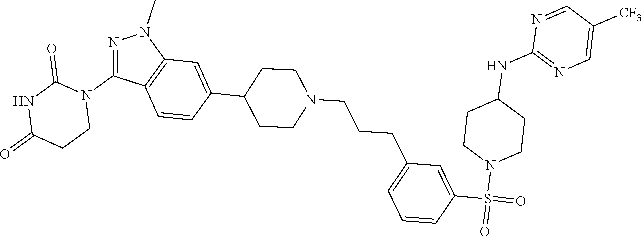 [SMILES: Cn1nc(N2CCC(=O)NC2=O)c2ccc(C3CCN(CCCc4cccc(S(=O)(=O)N5CCC(Nc6ncc(C(F)(F)F)cn6)CC5)c4)CC3)cc21] | 1-(1-methyl-6-(1-(3-(3-((4- ((5-(trifluoro- methyl)pyrimidin-2- yl)amino)piperidin-1- yl)sulfonyl)phenyl)propyl)p iperidin-4-yl)-1H-indazol-3- yl)dihydropyrimidine- 2,4(1H,3H)-dione | 754.1 |
| 88 | 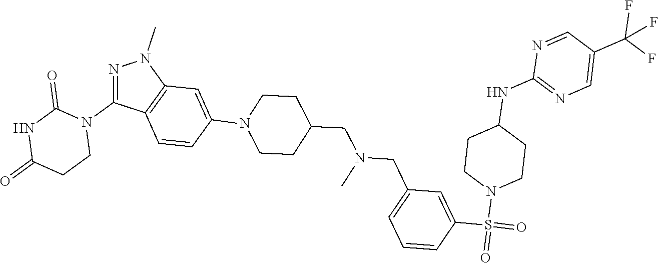 [SMILES: CN(Cc1cccc(S(=O)(=O)N2CCC(Nc3ncc(C(F)(F)F)cn3)CC2)c1)CC1CCN(c2ccc3c(N4CCC(=O)NC4=O)nn(C)c3c2)CC1] | 1-(1-methyl-6-(4- ((methyl(3-((4-((5- (trifluoromethyl)pyrimidin- 2-yl)amino)-piperidin-1- yl)sulfonyl)benzyl)amino)- methyl)piperidin-1-yl)-1H- indazol-3- yl)dihydropyrimidine- 2,4(1H,3H)-dione | 769.1 |
| 89 | 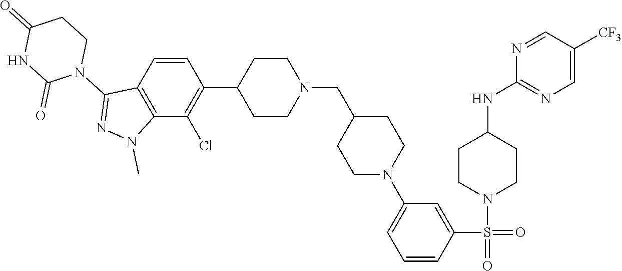 [SMILES: Cn1nc(N2CCC(=O)NC2=O)c2ccc(C3CCN(CC4CCN(c5cccc(S(=O)(=O)N6CCC(Nc7ncc(C(F)(F)F)cn7)CC6)c5)CC4)CC3)c(Cl)c21] | 1-(7-chloro-1-methyl-6-(1- ((1-(3-((4-((5- (trifluoromethyl)pyrimidin- 2-yl)amino)-piperidin-1- yl)sulfonyl)phenyl) piperidin-4-yl)methyl) piperidin-4- yl)-1H-indazol-3- yl)dihydropyrimidine- 2,4(1H,3H)-dione | 843.3 |
| 90 | 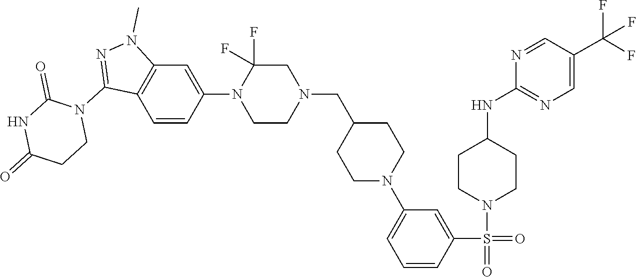 [SMILES: Cn1nc(N2CCC(=O)NC2=O)c2ccc(N3CCN(CC4CCN(c5cccc(S(=O)(=O)N6CCC(Nc7ncc(C(F)(F)F)cn7)CC6)c5)CC4)CC3(F)F)cc21] | 1-(6-(3,3-difluoro-1-((1-(3- ((4-((5- (trifluoromethyl)pyrimidin- 2-yl)amino)-piperidin-1- yl)sulfonyl)phenyl) piperidin-4-yl)methyl) piperidin-4- yl)-1-methyl-1H-indazol-3- yl)dihydropyrimidine- 2,4(1H,3H)-dione | 845.6 |
| 91 | 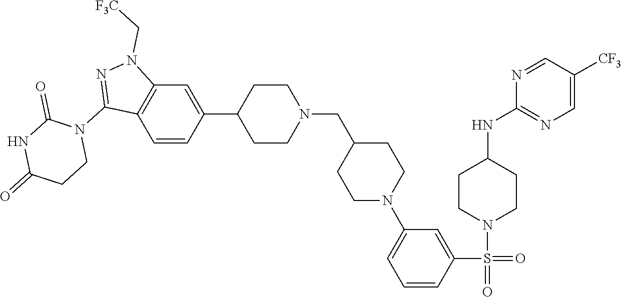 [SMILES: O=C1CCN(c2nn(CC(F)(F)F)c3cc(C4CCN(CC5CCN(c6cccc(S(=O)(=O)N7CCC(Nc8ncc(C(F)(F)F)cn8)CC7)c6)CC5)CC4)ccc23)C(=O)N1] | 1-(1-(2,2,2-trifluoroethyl)- 6-(1-((1-(3-((4-((5- (trifluoromethyl)pyrimidin- 2-yl)amino)piperidin-1- yl)sulfonyl)phenyl)- piperidin-4- yl)methyl)piperidin-4-yl)- 1H-indazol-3- yl)dihydropyrimidine- 2,4(1H,3H)-dione | 877.5 |
| 92 | 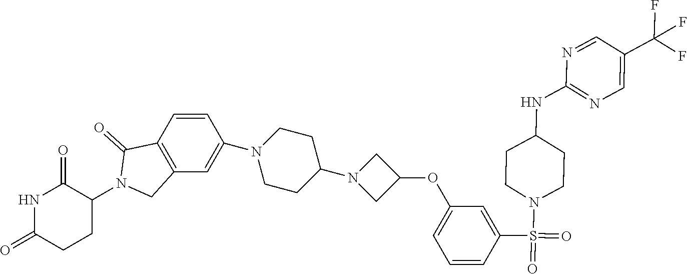 [SMILES: O=C1CCC(N2Cc3cc(N4CCC(N5CC(Oc6cccc(S(=O)(=O)N7CCC(Nc8ncc(C(F)(F)F)cn8)CC7)c6)C5)CC4)ccc3C2=O)C(=O)N1] | 3-(1-oxo-5-(4-(3-(3-((4-((5- (trifluoro-methyl)pyrimidin- 2-yl)amino)piperidin-1- yl)sulfonyl)phenoxy) azetidin-1-yl)piperidin-1- yl)isoindolin-2- yl)piperidine-2,6-dione | 783.5 |
| 93 | 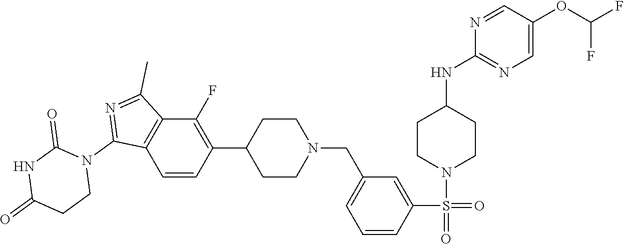 | 1-(6-(1-(3-((4-((5- (difluoromethoxy)- pyrimidin-2- yl)amino)piperidin-1- yl)sulfonyl)benzyl) piperidin-4-yl)-7- fluoro-1-methyl- 1H-indazol-3- yl)dihydropyrimidine- 2,4(1H,3H)-dione | 742.5 |
| 94 | 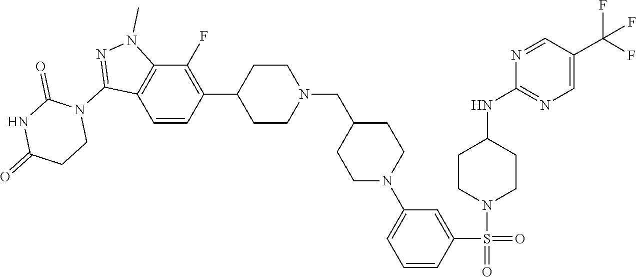 [SMILES: Cn1nc(N2CCC(=O)NC2=O)c2ccc(C3CCN(CC4CCN(c5cccc(S(=O)(=O)N6CCC(Nc7ncc(C(F)(F)F)cn7)CC6)c5)CC4)CC3)c(F)c21] | 1-(7-fluoro-1-methyl-6-(1- ((1-(3-((4-((5- (trifluoromethyl)pyrimidin- 2-yl)amino)-piperidin-1- yl)sulfonyl)phenyl) piperidin-4-yl)methyl) piperidin-4- yl)-1H-indazol-3- yl)dihydropyrimidine- 2,4(1H,3H)-dione | 827.6 |
| 95 | 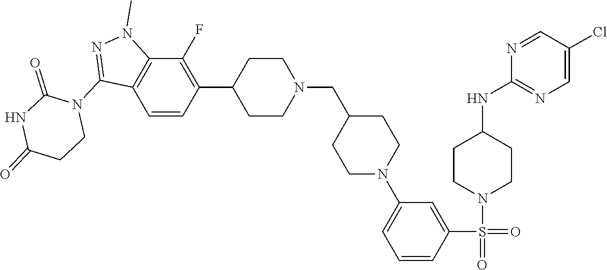 [SMILES: Cn1nc(N2CCC(=O)NC2=O)c2ccc(C3CCN(CC4CCN(c5cccc(S(=O)(=O)N6CCC(Nc7ncc(Cl)cn7)CC6)c5)CC4)CC3)c(F)c21] | 1-(6-(1-((1-(3-((4-((5- chloropyrimidin-2- yl)amino)piperidin-1- yl)sulfonyl)phenyl)- piperidin-4- yl)methyl)piperidin-4-yl)-7- fluoro-1-methyl-1H- indazol-3- yl)dihydropyrimidine- 2,4(1H,3H)-dione | 793.4 |
| 96 | 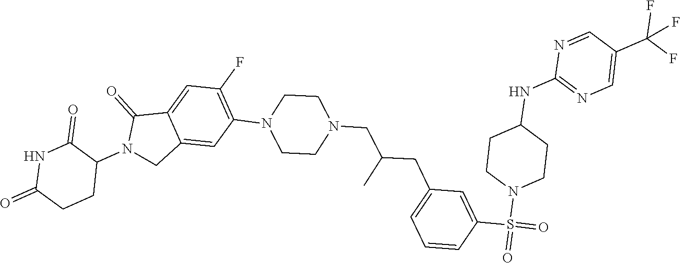 [SMILES: CC(Cc1cccc(S(=O)(=O)N2CCC(Nc3ncc(C(F)(F)F)cn3)CC2)c1)CN1CCN(c2cc3c(cc2F)C(=O)N(C2CCC(=O)NC2=O)C3)CC1] | 3-(6-fluoro-5-(4-(2-methyl- 3-(3-((4-((5- (trifluoromethyl)pyrimidin- 2-yl)amino)-piperidin-1- yl)sulfonyl)phenyl)propyl)- piperazin-1-yl)-1- oxoisoindolin-2- yl)piperidine-2,6-dione | 787.6 |
| 97 | 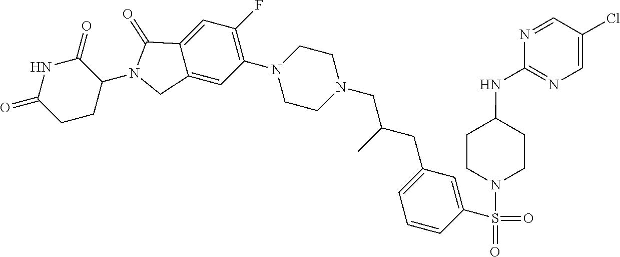 [SMILES: CC(Cc1cccc(S(=O)(=O)N2CCC(Nc3ncc(Cl)cn3)CC2)c1)CN1CCN(c2cc3c(cc2F)C(=O)N(C2CCC(=O)NC2=O)C3)CC1] | 3-(5-(4-(3-(3-((4-((5- chloropyrimidin-2- yl)amino)piperidin-1- yl)sulfonyl)phenyl)-2- methylpropyl)piperazin-1- yl)-6-fluoro-1- oxoisoindolin-2- yl)piperidine-2,6-dione | 753.1 |
| 98 | 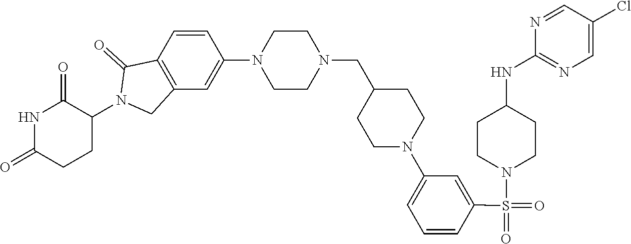 [SMILES: O=C1CCC(N2Cc3cc(N4CCN(CC5CCN(c6cccc(S(=O)(=O)N7CCC(Nc8ncc(Cl)cn8)CC7)c6)CC5)CC4)ccc3C2=O)C(=O)N1] | 3-(5-(4-((1-(3-((4-((5- chloropyrimidin-2- yl)amino)piperidin-1- yl)sulfonyl)phenyl)- piperidin-4- yl)methyl)piperazin-1-yl)-1- oxoisoindolin-2- yl)piperidine-2,6-dione | 776.3 |
| 99 | 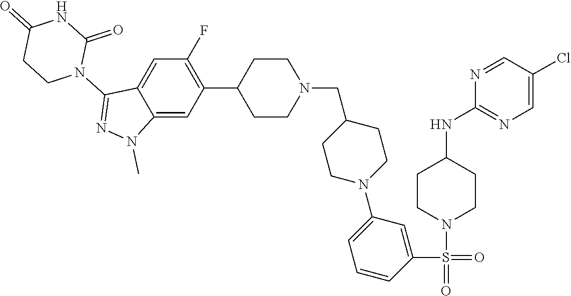 [SMILES: Cn1nc(N2CCC(=O)NC2=O)c2cc(F)c(C3CCN(CC4CCN(c5cccc(S(=O)(=O)N6CCC(Nc7ncc(Cl)cn7)CC6)c5)CC4)CC3)cc21] | 1-(6-(1-((1-(3-((4-((5- chloropyrimidin-2- yl)amino)piperidin-1- yl)sulfonyl)phenyl)- piperidin-4- yl)methyl)piperidin-4-yl)- 5-fluoro-1-methyl-1H- indazol-3- yl)dihydropyrimidine- 2,4(1H,3H)-dione | 793.4 |
| 100 | 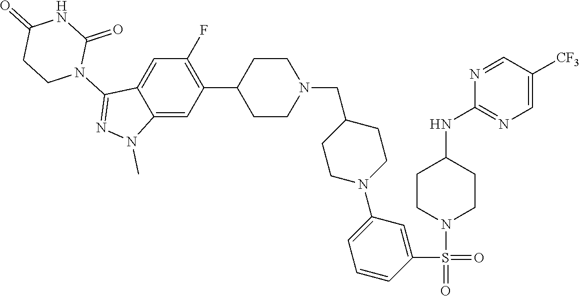 [SMILES: Cn1nc(N2CCC(=O)NC2=O)c2cc(F)c(C3CCN(CC4CCN(c5cccc(S(=O)(=O)N6CCC(Nc7ncc(C(F)(F)F)cn7)CC6)c5)CC4)CC3)cc21] | 1-(5-fluoro-1-methyl-6-(1- ((1-(3-((4-((5- (trifluoromethyl)pyrimidin- 2-yl)amino)-piperidin-1- yl)sulfonyl)phenyl) piperidin-4-yl)methyl) piperidin-4- yl)-1H-indazol-3- yl)dihydropyrimidine- 2,4(1H,3H)-dione | 827.6 |
| 101 | 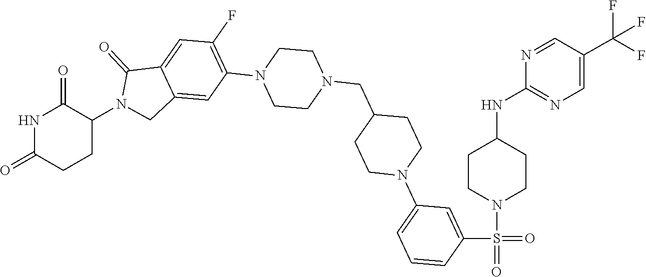 [SMILES: O=C1CCC(N2Cc3cc(N4CCN(CC5CCN(c6cccc(S(=O)(=O)N7CCC(Nc8ncc(C(F)(F)F)cn8)CC7)c6)CC5)CC4)c(F)cc3C2=O)C(=O)N1] | 3-(6-fluoro-1-oxo-5-(4-((1- (3-((4-((5- (trifluoromethyl)pyrimidin- 2-yl)amino)-piperidin-1- yl)sulfonyl)phenyl) piperidin-4-yl)methyl) piperazin-1- yl)isoindolin-2- yl)piperidine-2,6-dione | 828.0 |
| 102 | 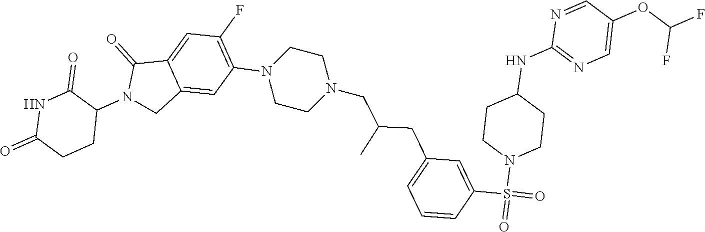 [SMILES: CC(Cc1cccc(S(=O)(=O)N2CCC(Nc3ncc(OC(F)F)cn3)CC2)c1)CN1CCN(c2cc3c(cc2F)C(=O)N(C2CCC(=O)NC2=O)C3)CC1] | 3-(5-(4-(3-(3-((4-((5- (difluoromethoxy)- pyrimidin-2- yl)amino)piperidin-1- yl)sulfonyl)phenyl)-2- methylpropyl)-piperazin- 1-yl)-6-fluoro-1- oxoisoindolin-2- yl)piperidine-2,6-dione | 785.1 |
| 103 | 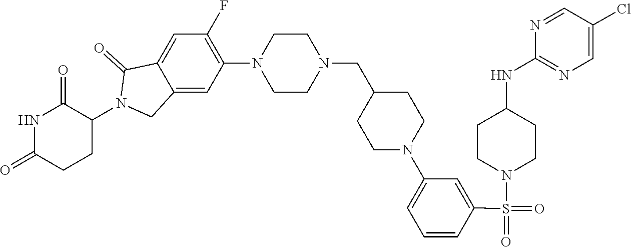 [SMILES: O=C1CCC(N2Cc3cc(N4CCN(CC5CCN(c6cccc(S(=O)(=O)N7CCC(Nc8ncc(Cl)cn8)CC7)c6)CC5)CC4)c(F)cc3C2=O)C(=O)N1] | 3-(5-(4-((1-(3-((4-((5- chloropyrimidin-2- yl)amino)piperidin-1- yl)sulfonyl)phenyl)- piperidin-4- yl)methyl)piperazin- 1-yl)-6-fluoro- 1-oxoisoindolin-2- yl)piperidine-2,6-dione | 794.0 |
| 104 | 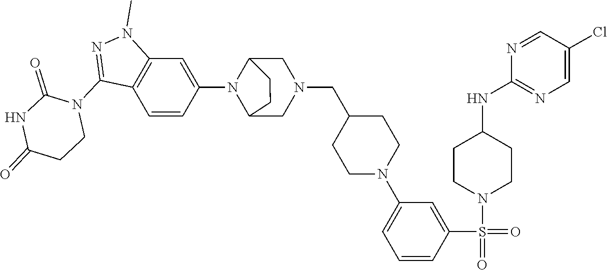 [SMILES: Cn1nc(N2CCC(=O)NC2=O)c2ccc(N3C4CCC3CN(CC3CCN(c5cccc(S(=O)(=O)N6CCC(Nc7ncc(Cl)cn7)CC6)c5)CC3)C4)cc21] | 1-(6-(3-((1-(3-((4-((5- chloropyrimidin-2- yl)amino)piperidin-1- yl)sulfonyl)phenyl)- piperidin-4-yl)methyl)-3,8- diaza-bicyclo[3.2.1]octan-8- yl)-1-methyl-1H-indazol-3- yl)dihydropyrimidine- 2,4(1H,3H)-dione | 802.1 |
| 105 | 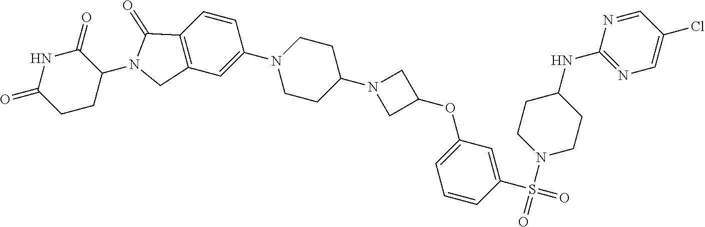 [SMILES: O=C1CCC(N2Cc3cc(N4CCC(N5CC(Oc6cccc(S(=O)(=O)N7CCC(Nc8ncc(Cl)cn8)CC7)c6)C5)CC4)ccc3C2=O)C(=O)N1] | 3-(5-(4-(3-(3-((4-((5- chloropyrimidin-2- yl)amino)piperidin-1- yl)sulfonyl)phenoxy)- azetidin-1-yl)piperidin-1- yl)-1-oxoisoindolin-2- yl)piperidine-2,6-dione | 749.1 |
| 106 | 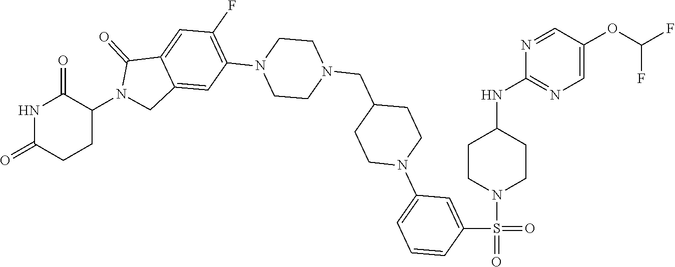 [SMILES: O=C1CCC(N2Cc3cc(N4CCN(CC5CCN(c6cccc(S(=O)(=O)N7CCC(Nc8ncc(OC(F)F)cn8)CC7)c6)CC5)CC4)c(F)cc3C2=O)C(=O)N1] | 3-(5-(4-((1-(3-((4-((5- (difluoromethoxy)- pyrimidin-2- yl)amino)piperidin-1- yl)sulfonyl)phenyl) piperidin-4-yl)methyl)- piperazin-1- yl)-6-fluoro-1- oxoisoindolin-2- yl)piperidine-2,6-dione | 826.5 |
| 107 | 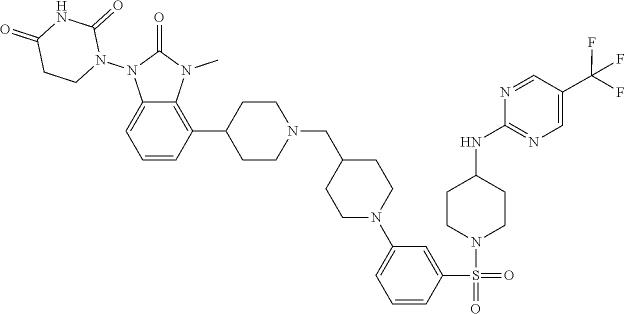 [SMILES: Cn1c(=O)n(N2CCC(=O)NC2=O)c2cccc(C3CCN(CC4CCN(c5cccc(S(=O)(=O)N6CCC(Nc7ncc(C(F)(F)F)cn7)CC6)c5)CC4)CC3)c21] | 3-(3-methyl-2-oxo-4-(1-((1- (3-((4-((5- (trifluoromethyl)pyrimidin- 2-yl)amino)-piperidin-1- yl)sulfonyl)phenyl) piperidin-4-yl)methyl) piperidin-4- yl)-2,3-dihydro-1H- benzo[d]imidazol-1- yl)piperidine-2,6-dione | 824.1 |
| 108 | 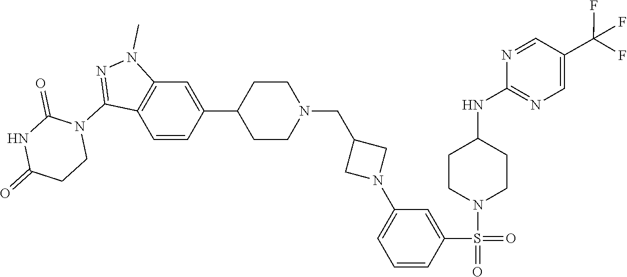 [SMILES: Cn1nc(N2CCC(=O)NC2=O)c2ccc(C3CCN(CC4CN(c5cccc(S(=O)(=O)N6CCC(Nc7ncc(C(F)(F)F)cn7)CC6)c5)C4)CC3)cc21] | 1-(1-methyl-6-(1-((1-(3-((4- ((5- (trifluoromethyl)pyrimidin- 2-yl)amino)-piperidin-1- yl)sulfonyl)phenyl)azetidin- 3-yl)methyl)piperidin-4-yl)- 1H-indazol-3- yl)dihydropyrimidine- 2,4(1H,3H)-dione | 781.1 |
| 109 | 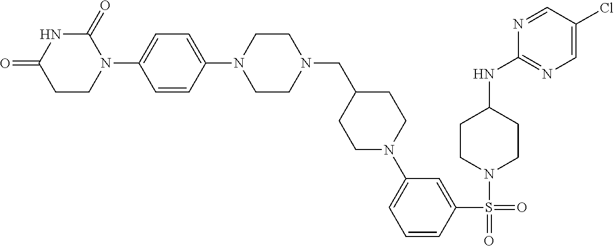 [SMILES: O=C1CCN(c2ccc(N3CCN(CC4CCN(c5cccc(S(=O)(=O)N6CCC(Nc7ncc(Cl)cn7)CC6)c5)CC4)CC3)cc2)C(=O)N1] | 1-(4-(4-((1-(3-((4-((5- chloropyrimidin-2- yl)amino)piperidin-1- yl)sulfonyl)phenyl)- piperidin-4- yl)methyl)piperazin-1- yl)phenyl) dihydropyrimidine- 2,4(1H,3H)-dione | 722.0 |
| 110 | 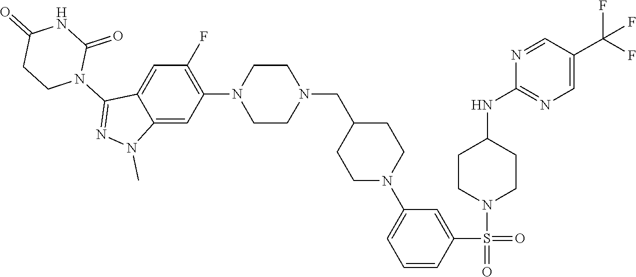 [SMILES: Cn1nc(N2CCC(=O)NC2=O)c2cc(F)c(N3CCN(CC4CCN(c5cccc(S(=O)(=O)N6CCC(Nc7ncc(C(F)(F)F)cn7)CC6)c5)CC4)CC3)cc21] | 1-(5-fluoro-1-methyl-6-(4- ((1-(3-((4-((5- (trifluoromethyl)pyrimidin- 2-yl)amino)-piperidin-1 - yl)sulfonyl)phenyl) piperidin-4-yl)methyl) piperazin-1- yl)-1H-indazol-3- yl)dihydropyrimidine- 2,4(1H,3H)-dione | 828.6 |
| 111 | 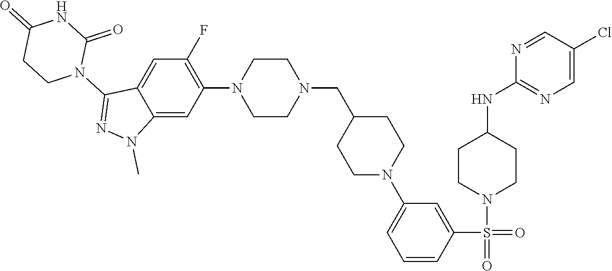 [SMILES: Cn1nc(N2CCC(=O)NC2=O)c2cc(F)c(N3CCN(CC4CCN(c5cccc(S(=O)(=O)N6CCC(Nc7ncc(Cl)cn7)CC6)c5)CC4)CC3)cc21] | 1-(6-(4-((1-(3-((4-((5- chloropyrimidin-2- yl)amino)piperidin-1- yl)sulfonyl)phenyl)- piperidin-4- yl)methyl)piperazin-1-yl)-5- fluoro-1-methyl-1H- indazol-3- yl)dihydropyrimidine- 2,4(1H,3H)-dione | 794.4 |
| 112 | 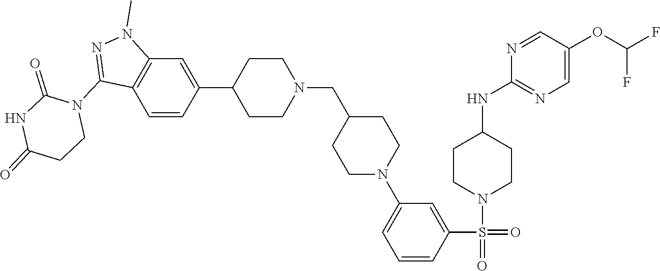 [SMILES: Cn1nc(N2CCC(=O)NC2=O)c2ccc(C3CCN(CC4CCN(c5cccc(S(=O)(=O)N6CCC(Nc7ncc(OC(F)F)cn7)CC6)c5)CC4)CC3)cc21] | 1-(6-(1-((1-(3-((4-((5- (difluoromethoxy)- pyrimidin-2- yl)amino)piperidin-1- yl)sulfonyl)phenyl) piperidin-4-yl)methyl) piperidin-4- yl)-1-methyl-1H-indazol-3- yl)dihydropyrimidine- 2,4(1H,3H)-dione | 807.6 |
| 113 | 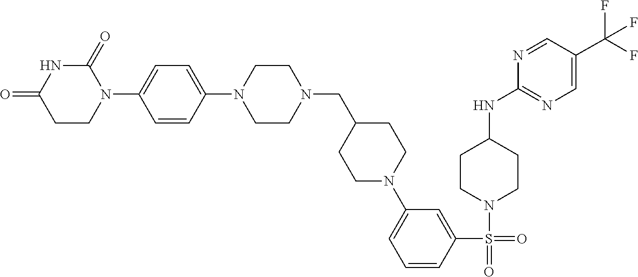 [SMILES: O=C1CCN(c2ccc(N3CCN(CC4CCN(c5cccc(S(=O)(=O)N6CCC(Nc7ncc(C(F)(F)F)cn7)CC6)c5)CC4)CC3)cc2)C(=O)N1] | 1-(4-(4-((1-(3-((4-((5- (trifluoromethyl)-pyrimidin- 2-yl)amino)piperidin-1- yl)sulfonyl)phenyl) piperidin-4-yl)methyl) piperazin-1- yl)phenyl)- dihydropyrimidine- 2,4(1H,3H)-dione | 756.1 |
| 114 | 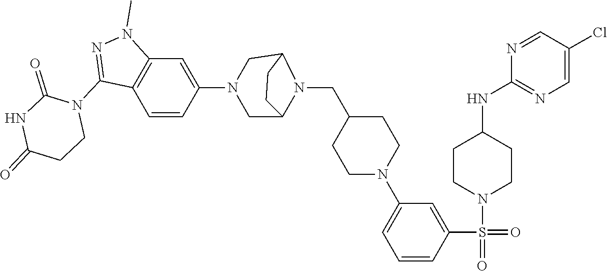 [SMILES: Cn1nc(N2CCC(=O)NC2=O)c2ccc(N3CC4CCC(C3)N4CC3CCN(c4cccc(S(=O)(=O)N5CCC(Nc6ncc(Cl)cn6)CC5)c4)CC3)cc21] | 1-(6-(8-((1-(3-((4-((5- chloropyrimidin-2- yl)amino)piperidin-1- yl)sulfonyl)phenyl)- piperidin-4-yl)methyl)-3,8- diaza-bicyclo[3.2.1]octan-3- yl)-1-methyl-1H-indazol-3- yl)dihydropyrimidine- 2,4(1H,3H)-dione | 802.0 |
| 115 | 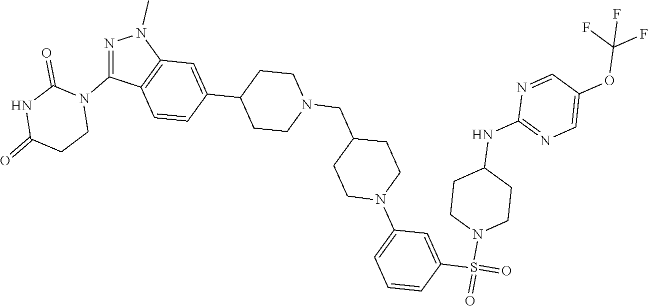 [SMILES: Cn1nc(N2CCC(=O)NC2=O)c2ccc(C3CCN(CC4CCN(c5cccc(S(=O)(=O)N6CCC(Nc7ncc(OC(F)(F)F)cn7)CC6)c5)CC4)CC3)cc21] | 1-(1-methyl-6-(1-((1-(3-((4- ((5-(trifluoro- methoxy)pyrimidin-2- yl)amino)piperidin-1- yl)sulfonyl)phenyl) piperidin-4-yl)methyl) piperidin-4- yl)-1H-indazol-3- yl)dihydropyrimidine- 2,4(1H,3H)-dione | 825.6 |
| 116 | 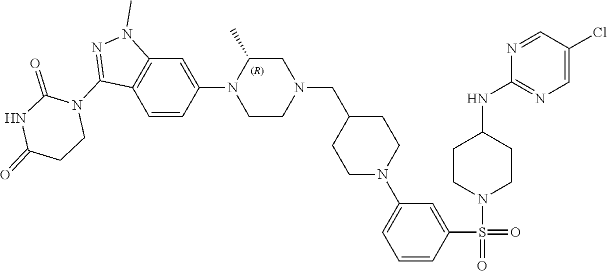 | (R)-1-(6-(4-((1-(3-((4-((5- chloropyrimidin-2- yl)amino)piperidin-1- yl)sulfonyl)phenyl)- piperidin-4-yl)methyl)-2- methylpiperazin-1-yl)-1- methyl-1H-indazol-3- yl)dihydropyrimidine- 2,4(1H,3H)-dione | 790.1 |
| 117 | 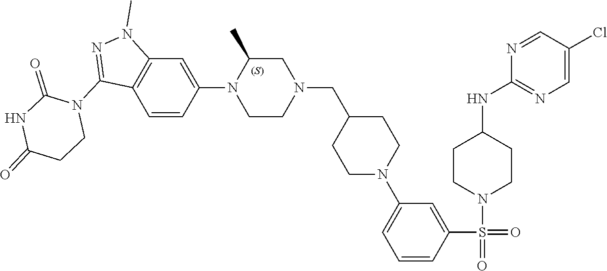 | (S)-1-(6-(4-((1-(3-((4-((5- chloropyrimidin-2- yl)amino)piperidin-1- yl)sulfonyl)phenyl)- piperidin-4-yl)methyl)-2- methylpiperazin-1-yl)-1- methyl-1H-indazol-3- yl)dihydro-pyrimidine- 2,4(1H,3H)-dione | 790.1 |
| 118 | 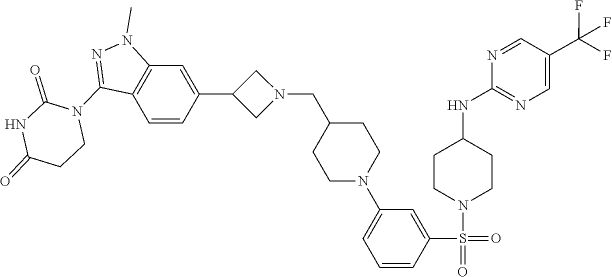 [SMILES: Cn1nc(N2CCC(=O)NC2=O)c2ccc(C3CN(CC4CCN(c5cccc(S(=O)(=O)N6CCC(Nc7ncc(C(F)(F)F)cn7)CC6)c5)CC4)C3)cc21] | 1-(1-methyl-6-(1-((1-(3-((4- ((5-(trifluoro- methyl)pyrimidin-2- yl)amino)piperidin-1- yl)sulfonyl)phenyl) piperidin-4-yl)methyl)- azetidin-3- yl)-1H-indazol-3- yl)dihydropyrimidine- 2,4(1H,3H)-dione | 781.1 |
| 119 | 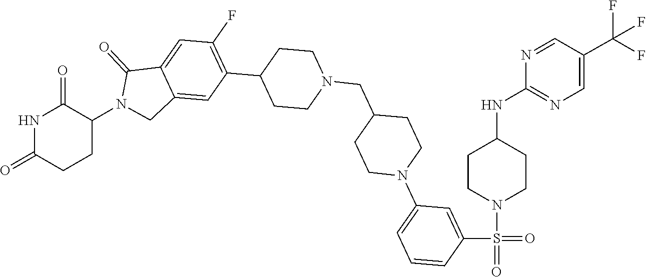 [SMILES: O=C1CCC(N2Cc3cc(C4CCN(CC5CCN(c6cccc(S(=O)(=O)N7CCC(Nc8ncc(C(F)(F)F)cn8)CC7)c6)CC5)CC4)c(F)cc3C2=O)C(=O)N1] | 3-(6-fluoro-1-oxo-5-(1-((1- (3-((4-((5- (trifluoromethyl)pyrimidin- 2-yl)amino)-piperidin-1- yl)sulfonyl)phenyl) piperidin-4-yl)methyl) piperidin-4- yl)isoindolin-2- yl)piperidine-2,6-dione | 827.1 |
| 120 | 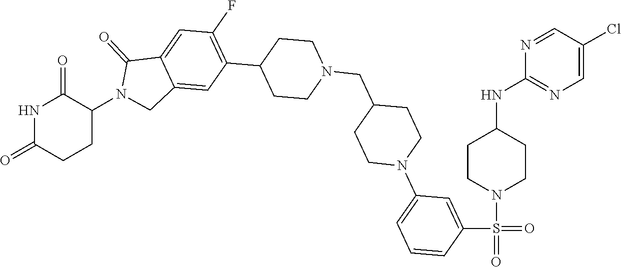 [SMILES: O=C1CCC(N2Cc3cc(C4CCN(CC5CCN(c6cccc(S(=O)(=O)N7CCC(Nc8ncc(Cl)cn8)CC7)c6)CC5)CC4)c(F)cc3C2=O)C(=O)N1] | 3-(5-(1-((1-(3-((4-((5- chloropyrimidin-2- yl)amino)piperidin-1- yl)sulfonyl)phenyl)- piperidin-4- yl)methyl)piperidin-4-yl)-6- fluoro-1-oxoisoindolin-2- yl)piperidine-2,6-dione | 793.1 |
| 121 | 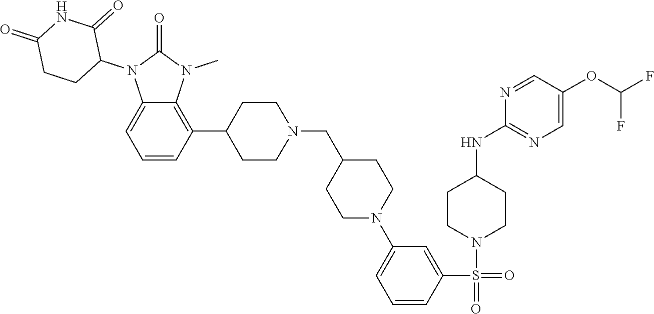 [SMILES: Cn1c(=O)n(C2CCC(=O)NC2=O)c2cccc(C3CCN(CC4CCN(c5cccc(S(=O)(=O)N6CCC(Nc7ncc(OC(F)F)cn7)CC6)c5)CC4)CC3)c21] | 3-(4-(1-((1-(3-((4-((5- (difluoromethoxy)- pyrimidin-2- yl)amino)piperidin-1- yl)sulfonyl)phenyl) piperidin-4-yl)methyl)- piperidin-4- yl)-3-methyl-2-oxo-2,3- dihydro-1H- benzo[d]imidazol-1- yl)piperidine-2,6-dione | 822.6 |
| 122 | 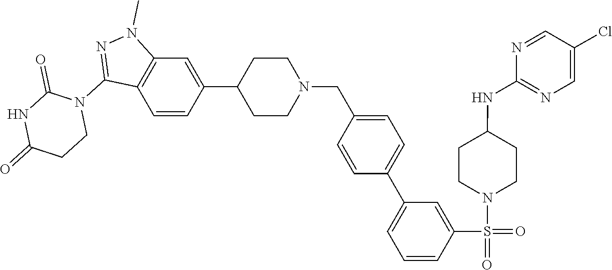 [SMILES: Cn1nc(N2CCC(=O)NC2=O)c2ccc(C3CCN(Cc4ccc(-c5cccc(S(=O)(=O)N6CCC(Nc7ncc(Cl)cn7)CC6)c5)cc4)CC3)cc21] | 1-(6-(1-((3'-((4-((5- chloropyrimidin-2- yl)amino)piperidin-1- yl)sulfonyl)-[1,1'-bipheny]]- 4-yl)methyl)piperidin-4-yl)- 1-methyl-1H-indazol-3- yl)dihydropyrimidine- 2,4(1H,3H)-dione | 768.1 |
| 123 | 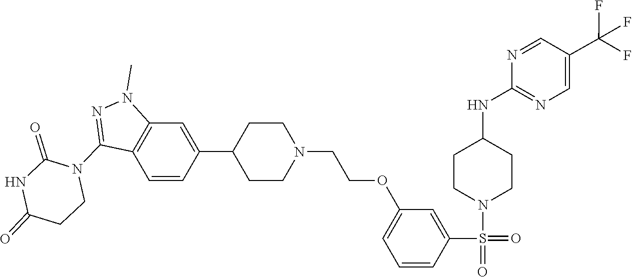 [SMILES: Cn1nc(N2CCC(=O)NC2=O)c2ccc(C3CCN(CCOc4cccc(S(=O)(=O)N5CCC(Nc6ncc(C(F)(F)F)cn6)CC5)c4)CC3)cc21] | 1-(1-methyl-6-(1-(2-(3-((4- ((5-(trifluoro- methyl)pyrimidin-2- yl)amino)piperidin-1- yl)sulfonyl)phenoxy)ethyl) piperidin-4-yl)- 1H-indazol-3- yl)dihydropyrimidine- 2,4(1H,3H)-dione | 756.1 |
| 124 | 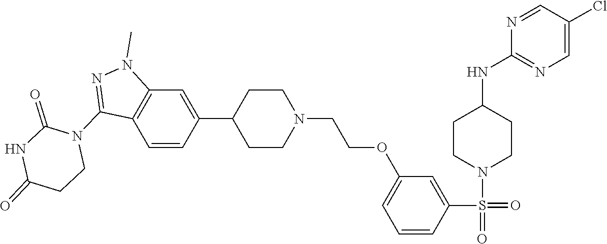 [SMILES: Cn1nc(N2CCC(=O)NC2=O)c2ccc(C3CCN(CCOc4cccc(S(=O)(=O)N5CCC(Nc6ncc(Cl)cn6)CC5)c4)CC3)cc21] | 1-(6-(1-(2-(3-((4-((5- chloropyrimidin-2- yl)amino)piperidin-1- yl)sulfonyl)phenoxy)- ethyl)piperidin-4-yl)-1- methyl-1H-indazol-3- yl)dihydropyrimidine- 2,4(1H,3H)-dione | 722.1 |
| 125 | 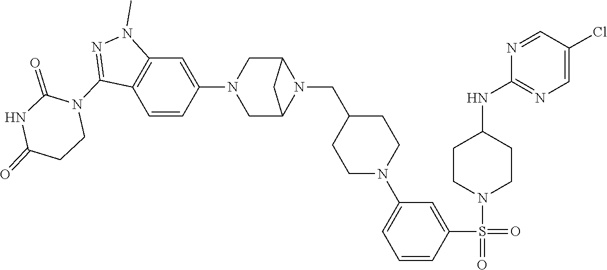 [SMILES: Cn1nc(N2CCC(=O)NC2=O)c2ccc(N3CC4CC(C3)N4CC3CCN(c4cccc(S(=O)(=O)N5CCC(Nc6ncc(Cl)cn6)CC5)c4)CC3)cc21] | 1-(6-(6-((1-(3-((4-((5- chloropyrimidin-2- yl)amino)piperidin-1- yl)sulfonyl)phenyl)- piperidin-4-yl)methyl)-3,6- diazabicyclo-[3.1.1]heptan- 3-yl)-1-methyl-1H-indazol- 3-yl)dihydropyrimidine- 2,4(1H,3H)-dione | 788.4 |
| 126 | 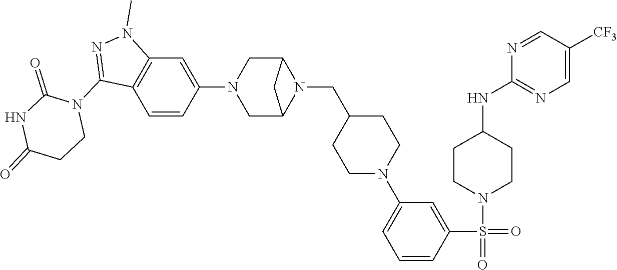 [SMILES: Cn1nc(N2CCC(=O)NC2=O)c2ccc(N3CC4CC(C3)N4CC3CCN(c4cccc(S(=O)(=O)N5CCC(Nc6ncc(C(F)(F)F)cn6)CC5)c4)CC3)cc21] | 1-(1-methyl-6-(6-((1-(3-((4- ((5-(trifluoro- methyl)pyrimidin-2- yl)amino)piperidin-1- yl)sulfonyl)phenyl) piperidin-4-yl)methyl)-3,6- diazabicyclo[3.1.1]heptan- 3-yl)-1H-indazol-3- yl)dihydropyrimidine- 2,4(1H,3H)-dione | 822.6 |
| 127 | 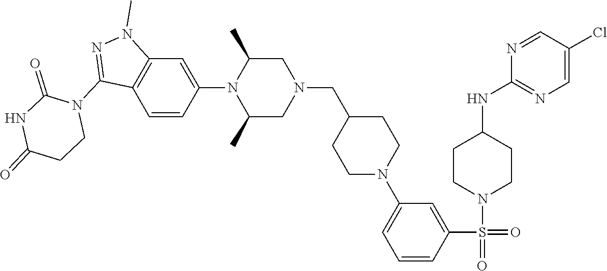 [SMILES: C[C@@H]1CN(CC2CCN(c3cccc(S(=O)(=O)N4CCC(Nc5ncc(Cl)cn5)CC4)c3)CC2)C[C@H](C)N1c1ccc2c(N3CCC(=O)NC3=O)nn(C)c2c1] | 1-(6-((2S,6R)-4-((1-(3-((4- ((5-chloro-pyrimidin-2- yl)amino)piperidin-1-yl)- sulfonyl)phenyl)piperidin-4- yl)methyl)-2,6- dimethylpiperazin-1-yl)-1- methyl-1H-indazol-3- yl)dihydropyrimidine- 2,4(1H,3H)-dione | 804.4 |
| 128 | 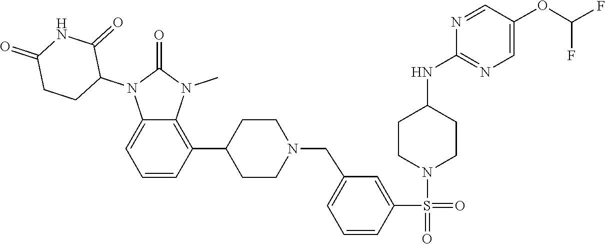 [SMILES: Cn1c(=O)n(C2CCC(=O)NC2=O)c2cccc(C3CCN(Cc4cccc(S(=O)(=O)N5CCC(Nc6ncc(OC(F)F)cn6)CC5)c4)CC3)c21] | 3-(4-(1-(3-((4-((5- (difluoromethoxy)- pyrimidin-2- yl)amino)piperidin-1- yl)sulfonyl)benzyl) piperidin-4-yl)-3- methyl-2-oxo-2,3- dihydro-1H- benzo[d]imidazol-1- yl)piperidine-2,6-dione | 739.1 |
| 129 | 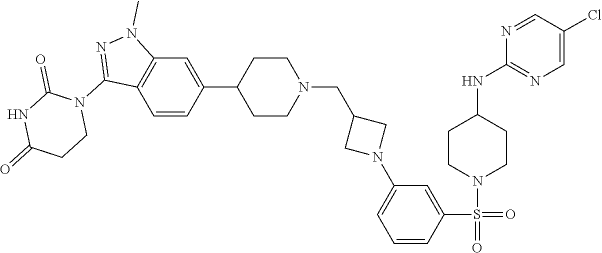 [SMILES: Cn1nc(N2CCC(=O)NC2=O)c2ccc(C3CCN(CC4CN(c5cccc(S(=O)(=O)N6CCC(Nc7ncc(Cl)cn7)CC6)c5)C4)CC3)cc21] | 1-(6-(1-((1-(3-((4-((5- chloropyrimidin-2- yl)amino)piperidin-1- yl)sulfonyl)phenyl)- azetidin-3- yl)methyl)piperidin-4-yl)- 1-methyl-1H-indazol-3- yl)dihydropyrimidine- 2,4(1H,3H)-dione | 747.1 |
| 130 | 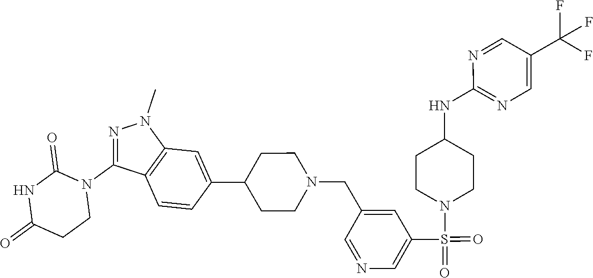 [SMILES: Cn1nc(N2CCC(=O)NC2=O)c2ccc(C3CCN(Cc4cncc(S(=O)(=O)N5CCC(Nc6ncc(C(F)(F)F)cn6)CC5)c4)CC3)cc21] | 1-(1-methyl-6-(1-((5-((4- ((5-(trifluoro- methyl)pyrimidin-2- yl)amino)piperidin-1- yl)sulfonyl)pyridin-3- yl)methyl)piperidin-4-yl)- 1H-indazol-3- yl)dihydropyrimidine- 2,4(1H,3H)-dione | 727.0 |
| 131 | 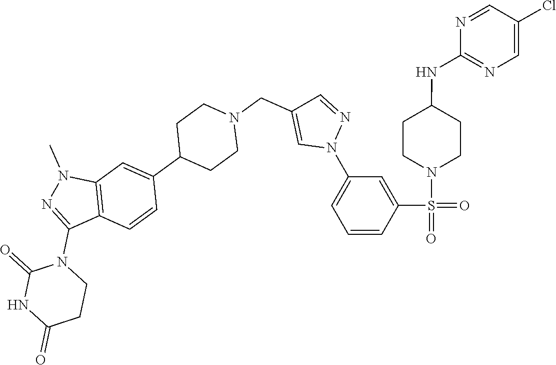 [SMILES: Cn1nc(N2CCC(=O)NC2=O)c2ccc(C3CCN(Cc4cnn(-c5cccc(S(=O)(=O)N6CCC(Nc7ncc(Cl)cn7)CC6)c5)c4)CC3)cc21] | 1-(6-(1-((1-(3-((4-((5- chloropyrimidin-2- yl)amino)piperidin-1- yl)sulfonyl)phenyl)-1H- pyrazol-4- yl)methyl)piperidin-4-yl)- 1-methyl-1H-indazol-3- yl)dihydropyrimidine- 2,4(1H,3H)-dione | 758.1 |
| 132 | 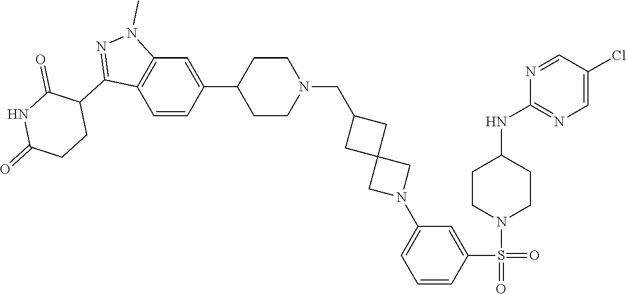 [SMILES: Cn1nc(C2CCC(=O)NC2=O)c2ccc(C3CCN(CC4CC5(C4)CN(c4cccc(S(=O)(=O)N6CCC(Nc7ncc(Cl)cn7)CC6)c4)C5)CC3)cc21] | 1-(6-(1-((2-(3-((4-((5- chloropyrimidin-2- yl)amino)piperidin-1- yl)sulfonyl)phenyl)-2- azaspiro[3.3]heptan-6- yl)methyl)piperidin-4-yl)- 1-methyl-1H-indazol-3- yl)dihydropyrimidine- 2,4(1H,3H)-dione | 787.1 |
| 133 | 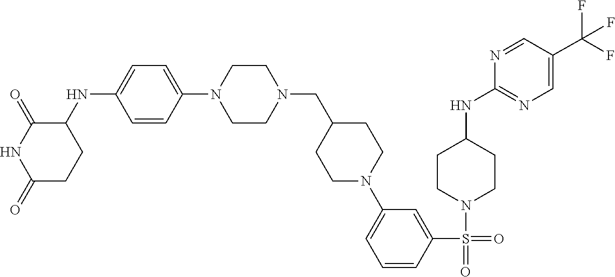 [SMILES: O=C1CCC(Nc2ccc(N3CCN(CC4CCN(c5cccc(S(=O)(=O)N6CCC(Nc7ncc(C(F)(F)F)cn7)CC6)c5)CC4)CC3)cc2)C(=O)N1] | 3-((4-(4-((1-(3-((4-((5- (trifluoromethyl)-pyrimidin- 2-yl)amino)piperidin-1- yl)sulfonyl)phenyl) piperidin-4-yl)methyl) piperazin-1- yl)phenyl)amino)- piperidine-2,6-dione | 770.1 |
| 134 | 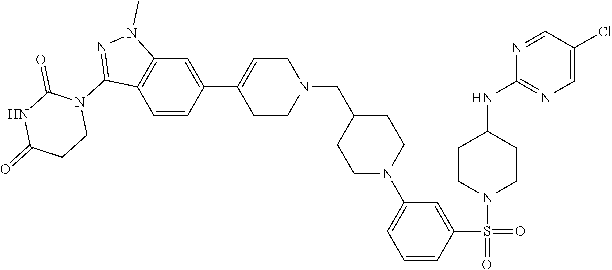 [SMILES: Cn1nc(N2CCC(=O)NC2=O)c2ccc(C3=CCN(CC4CCN(c5cccc(S(=O)(=O)N6CCC(Nc7ncc(Cl)cn7)CC6)c5)CC4)CC3)cc21] | 1-(6-(1-((1-(3-((4-((5- chloropyrimidin-2- yl)amino)piperidin-1- yl)sulfonyl)phenyl)- piperidin-4-yl)methyl)- 1,2,3,6-tetrahydropyridin-4- yl)-1-methyl-1H-indazol-3- yl)dihydropyrimidine- 2,4(1H,3H)-dione | 773.1 |
| 135 | 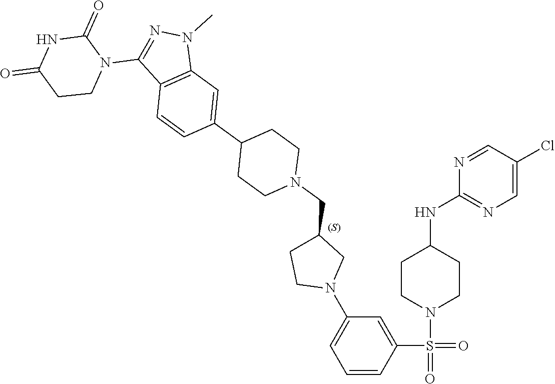 | (S)-1-(6-(1-((1-(3-((4-((5- chloropyrimidin-2- yl)amino)piperidin-1- yl)sulfonyl)phenyl)- pyrrolidin-3- yl)methyl)piperidin-4-yl)- 1-methyl-1H-indazol-3- yl)dihydropyrimidine- 2,4(1H,3H)-dione | 762.1 |
| 136 | 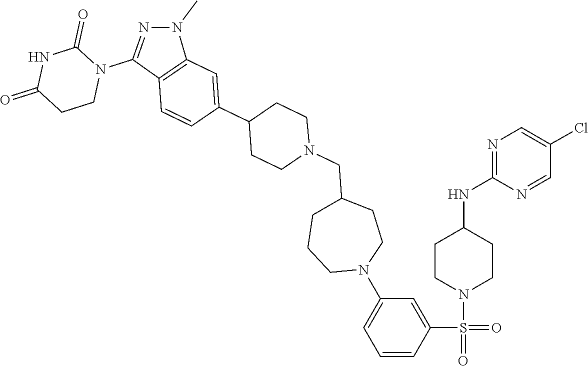 [SMILES: Cn1nc(N2CCC(=O)NC2=O)c2ccc(C3CCN(CC4CCCN(c5cccc(S(=O)(=O)N6CCC(Nc7ncc(Cl)cn7)CC6)c5)CC4)CC3)cc21] | 1-(6-(1-((1-(3-((4-((5- chloropyrimidin-2- yl)amino)piperidin-1- yl)sulfonyl)phenyl)-azepan- 4-yl)methyl)piperidin-4-yl)- 1-methyl-1H-indazol-3- yl)dihydropyrimidine- 2,4(1H,3H)-dione | 789.1 |
| 137 | 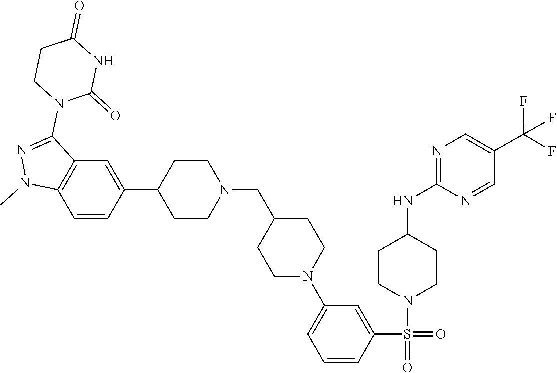 [SMILES: Cn1nc(N2CCC(=O)NC2=O)c2cc(C3CCN(CC4CCN(c5cccc(S(=O)(=O)N6CCC(Nc7ncc(C(F)(F)F)cn7)CC6)c5)CC4)CC3)ccc21] | 1-(1-methyl-5-(1-((1-(3-((4- ((5-(trifluoro- methyl)pyrimidin-2- yl)amino)piperidin-1- yl)sulfonyl)phenyl) piperidin-4-yl)methyl)- piperidin-4- yl)-1H-indazol-3- yl)dihydropyrimidine- 2,4(1H,3H)-dione | 809.3 |
| 138 | 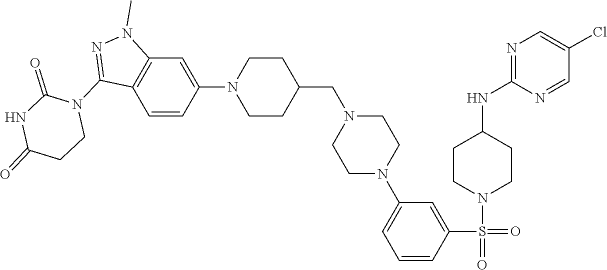 [SMILES: Cn1nc(N2CCC(=O)NC2=O)c2ccc(N3CCC(CN4CCN(c5cccc(S(=O)(=O)N6CCC(Nc7ncc(Cl)cn7)CC6)c5)CC4)CC3)cc21] | 1-(6-(4-((4-(3-((4-((5- chloropyrimidin-2- yl)amino)piperidin-1- yl)sulfonyl)phenyl)- piperazin-1- yl)methyl)piperidin-1-yl)- 1-methyl-1H-indazol-3- yl)dihydropyrimidine- 2,4(1H,3H)-dione | 776.3 |
| 139 | 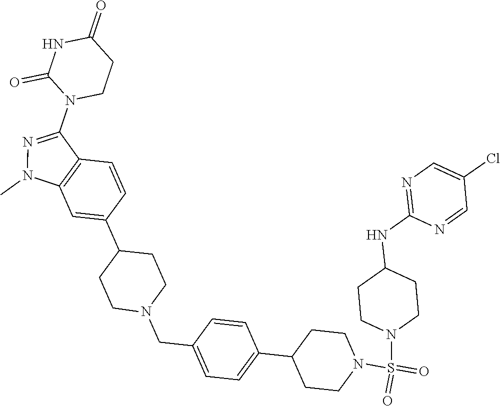 [SMILES: Cn1nc(N2CCC(=O)NC2=O)c2ccc(C3CCN(Cc4ccc(C5CCN(S(=O)(=O)N6CCC(Nc7ncc(Cl)cn7)CC6)CC5)cc4)CC3)cc21] | 1-(6-(1-(4-(1-((4-((5- chloropyrimidin-2- yl)amino)piperidin-1- yl)sulfonyl)piperidin-4- yl)benzyl)piperidin-4-yl)- 1-methyl-1H-indazol-3- yl)dihydropyrimidine- 2,4(1H,3H)-dione | 775.3 |
| 140 | 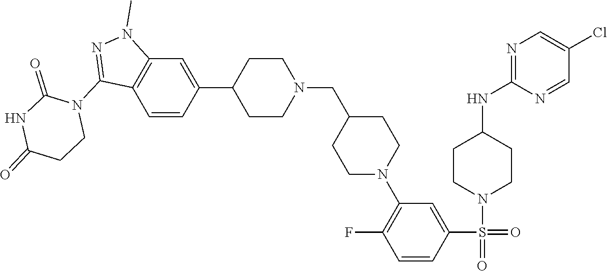 [SMILES: Cn1nc(N2CCC(=O)NC2=O)c2ccc(C3CCN(CC4CCN(c5cc(S(=O)(=O)N6CCC(Nc7ncc(Cl)cn7)CC6)ccc5F)CC4)CC3)cc21] | 1-(6-(1-((1-(5-((4-((5- chloropyrimidin-2- yl)amino)piperidin-1- yl)sulfonyl)-2- fluorophenyl)piperidin-4- yl)methyl)-piperidin-4-yl)- 1-methyl-1H-indazol-3- yl)dihydropyrimidine- 2,4(1H,3H)-dione | 793.1 |
| 141 | 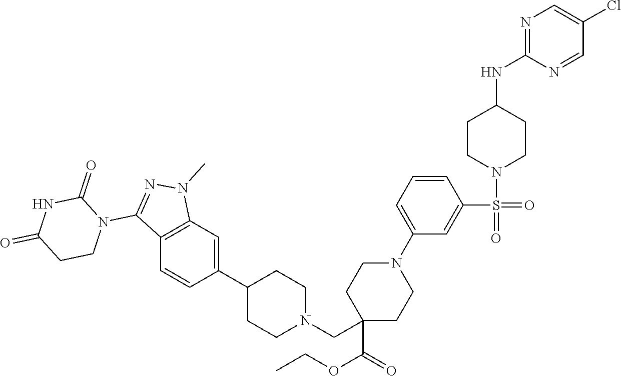 [SMILES: CCOC(=O)C1(CN2CCC(c3ccc4c(N5CCC(=O)NC5=O)nn(C)c4c3)CC2)CCN(c2cccc(S(=O)(=O)N3CCC(Nc4ncc(Cl)cn4)CC3)c2)CC1] | ethyl 1-(3-((4-((5- chloropyrimidin-2- yl)amino)piperidin-1- yl)sulfonyl)phenyl)-4-((4- (3-(2,4- dioxotetrahydropyrimidin- 1(2H)-yl)-1-methyl-1H- indazol-6-yl)piperidin-1- yl)methyl)piperidine-4- carboxylate | 847.3 |
| 142 | 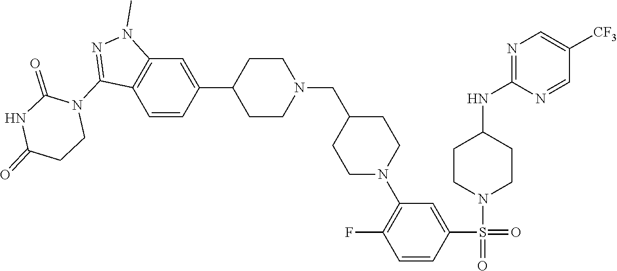 [SMILES: Cn1nc(N2CCC(=O)NC2=O)c2ccc(C3CCN(CC4CCN(c5cc(S(=O)(=O)N6CCC(Nc7ncc(C(F)(F)F)cn7)CC6)ccc5F)CC4)CC3)cc21] | 1-(6-(1-((1-(2-fluoro-5-((4- ((5-(trifluoro- methyl)pyrimidin-2- yl)amino)piperidin-1- yl)sulfonyl)phenyl) piperidin-4-yl)methyl)- piperidin-4- yl)-1-methyl-1H-indazol- 3-yl)dihydropyrimidine- 2,4(1H,3H)-dione | 827.1 |
| 143 | 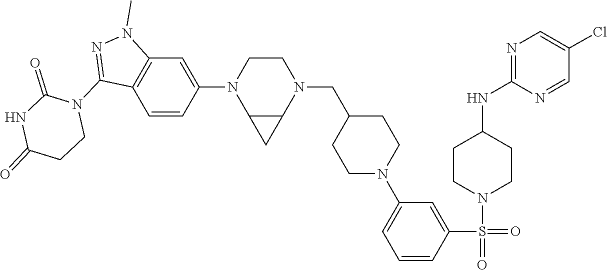 [SMILES: Cn1nc(N2CCC(=O)NC2=O)c2ccc(N3CCN(CC4CCN(c5cccc(S(=O)(=O)N6CCC(Nc7ncc(Cl)cn7)CC6)c5)CC4)C4CC43)cc21] | 1-(6-(5-((1-(3-((4-((5- chloropyrimidin-2- yl)amino)piperidin-1- yl)sulfonyl)phenyl)- piperidin-4-yl)methyl)-2,5- diazabicyclo-[4.1.0]heptan- 2-yl)-1-methyl-1H-indazol- 3-yl)dihydropyrimidine- 2,4(1H,3H)-dione | 788.3 |
| 144 | 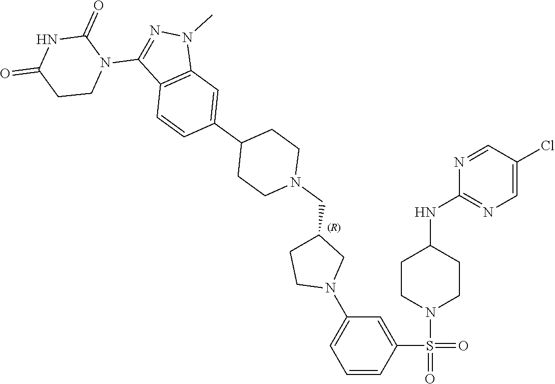 | (R)-1-(6-(1-((1-(3-((4-((5- chloropyrimidin-2- yl)amino)piperidin-1- yl)sulfonyl)phenyl)- pyrrolidin-3- yl)methyl)piperidin-4-yl)- 1-methyl-1H-indazol-3- yl)dihydropyrimidine- 2,4(1H,3H)-dione | 761.1 |
| 145 | 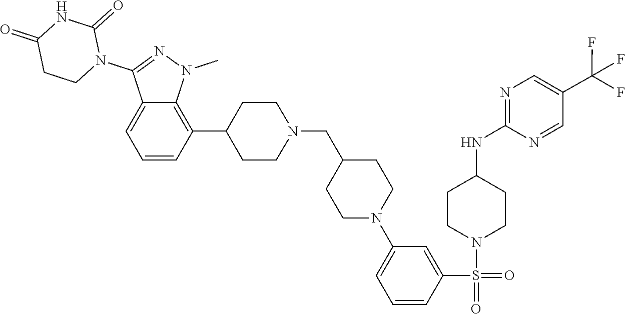 [SMILES: Cn1nc(N2CCC(=O)NC2=O)c2cccc(C3CCN(CC4CCN(c5cccc(S(=O)(=O)N6CCC(Nc7ncc(C(F)(F)F)cn7)CC6)c5)CC4)CC3)c21] | 1-(1-methyl-7-(1-((1-(3-((4- ((5-(trifluoro- methyl)pyrimidin-2- yl)amino)piperidin-1- yl)sulfonyl)phenyl) piperidin-4-yl)methyl)- piperidin-4- yl)-1H-indazol-3- yl)dihydropyrimidine- 2,4(1H,3H)-dione | 809.1 |
| 146 | 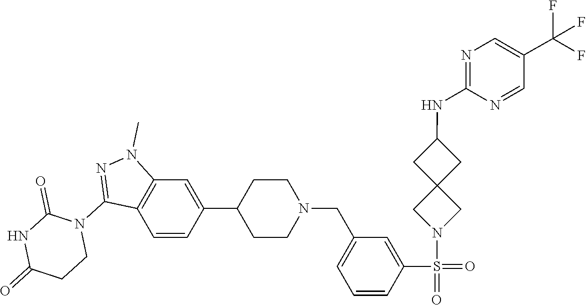 [SMILES: Cn1nc(N2CCC(=O)NC2=O)c2ccc(C3CCN(Cc4cccc(S(=O)(=O)N5CC6(CC(Nc7ncc(C(F)(F)F)cn7)C6)C5)c4)CC3)cc21] | 1-(1-methyl-6-(1-(3-((6-((5- (trifluoro-methyl)pyrimidin- 2-yl)amino)-2- azaspiro[3.3]heptan-2- yl)sulfonyl)benzyl)- piperidin-4-yl)-1H-indazol- 3-yl)dihydropyrimidine- 2,4(1H,3H)-dione | 738.1 |
| 147 | 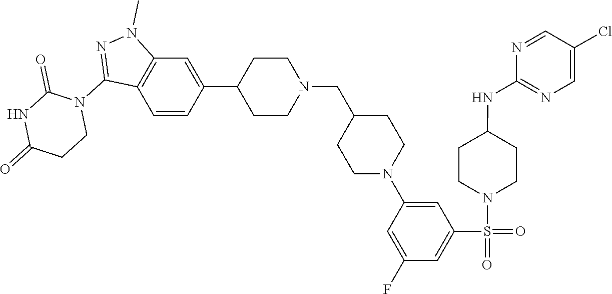 [SMILES: Cn1nc(N2CCC(=O)NC2=O)c2ccc(C3CCN(CC4CCN(c5cc(F)cc(S(=O)(=O)N6CCC(Nc7ncc(Cl)cn7)CC6)c5)CC4)CC3)cc21] | 1-(6-(1-((1-(3-((4-((5- chloropyrimidin-2- yl)amino)piperidin-1- yl)sulfonyl)-5- fluorophenyl)piperidin-4- yl)methyl)piperidin-4-yl)- 1-methyl-1H-indazol-3- yl)dihydropyrimidine- 2,4(1H,3H)-dione | 793.1 |
| 148 | 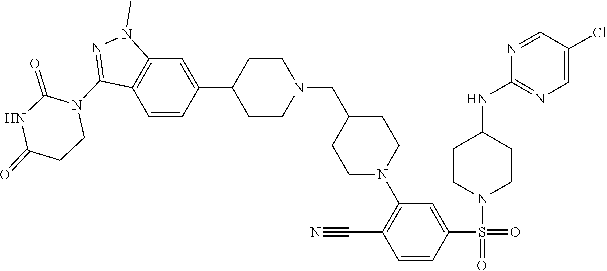 [SMILES: Cn1nc(N2CCC(=O)NC2=O)c2ccc(C3CCN(CC4CCN(c5cc(S(=O)(=O)N6CCC(Nc7ncc(Cl)cn7)CC6)ccc5C#N)CC4)CC3)cc21] | 4-((4-((5-chloropyrimidin- 2-yl)amino)piperidin-1- yl)sulfonyl)-2-(4-((4-(3- (2,4- dioxotetrahydropyrimidin- 1(2H)-yl)-1-methyl-1H- indazol-6-yl)piperidin-1- yl)methyl)piperidin-1- yl)benzonitrile | 800.1 |
| 149 | 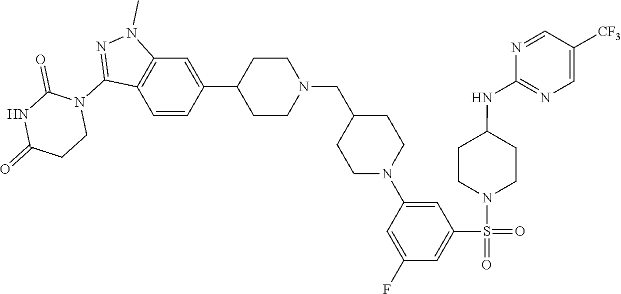 [SMILES: Cn1nc(N2CCC(=O)NC2=O)c2ccc(C3CCN(CC4CCN(c5cc(F)cc(S(=O)(=O)N6CCC(Nc7ncc(C(F)(F)F)cn7)CC6)c5)CC4)CC3)cc21] | 1-(6-(1-((1-(3-fluoro-5-((4- ((5- (trifluoromethyl)pyrimidin- 2-yl)amino)piperidin-1- yl)sulfonyl)phenyl) piperidin-4-yl)methyl) piperidin-4- yl)-1-methyl-1H-indazol- 3-yl)dihydropyrimidine- 2,4(1H,3H)-dione | 827.1 |
| 150 | 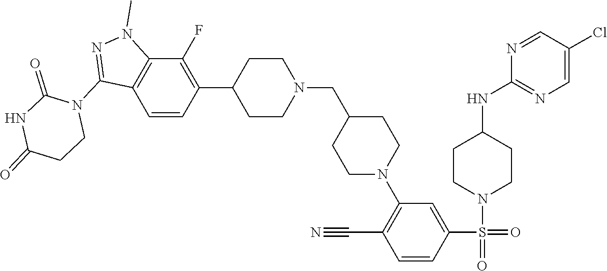 [SMILES: Cn1nc(N2CCC(=O)NC2=O)c2ccc(C3CCN(CC4CCN(c5cc(S(=O)(=O)N6CCC(Nc7ncc(Cl)cn7)CC6)ccc5C#N)CC4)CC3)c(F)c21] | 4-((4-((5-chloropyrimidin- 2-yl)amino)piperidin-1- yl)sulfonyl)-2-(4-((4-(3- (2,4- dioxotetrahydropyrimidin- 1(2H)-yl)-7-fluoro-1- methyl-1H-indazol-6- yl)piperidin-1- yl)methyl)piperidin-1- yl)benzonitrile | 819.1 |
| 151 | 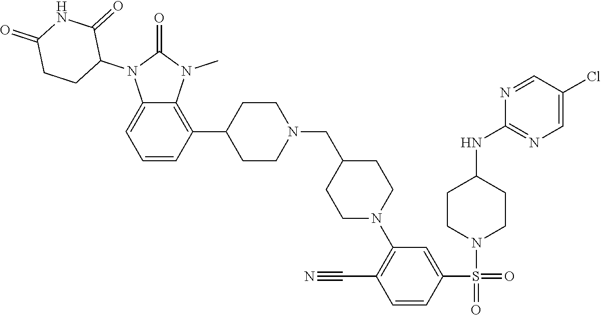 [SMILES: Cn1c(=O)n(C2CCC(=O)NC2=O)c2cccc(C3CCN(CC4CCN(c5cc(S(=O)(=O)N6CCC(Nc7ncc(Cl)cn7)CC6)ccc5C#N)CC4)CC3)c21] | 4-((4-((5-chloropyrimidin- 2-yl)amino)piperidin-1- yl)sulfonyl)-2-(4-((4-(1- (2,6-dioxopiperidin-3-yl)-3- methyl-2-oxo-2,3-dihydro- 1H-benzo[d]imidazol-4- yl)piperidin-1- yl)methyl)piperidin-1- yl)benzonitrile | 815.1 |
| 152 | 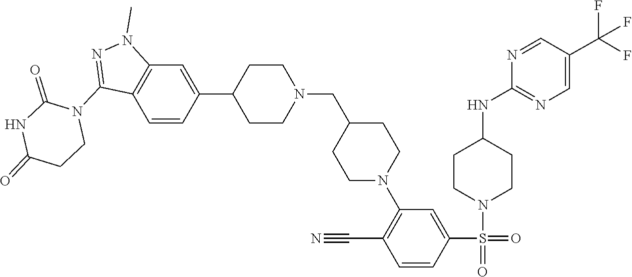 [SMILES: Cn1nc(N2CCC(=O)NC2=O)c2ccc(C3CCN(CC4CCN(c5cc(S(=O)(=O)N6CCC(Nc7ncc(C(F)(F)F)cn7)CC6)ccc5C#N)CC4)CC3)cc21] | 2-(4-((4-(3-(2,4- dioxotetrahydropyrimidin- 1(2H)-yl)-1-methyl-1H- indazol-6-yl)piperidin-1- yl)methyl)piperidin-1-yl)- 4-((4-((5- (trifluoromethyl)pyrimidin- 2-yl)amino)piperidin-1- yl)sulfonyl)benzonitrile | 834.1 |
| 153 | 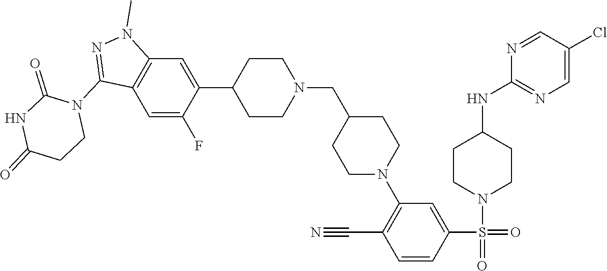 [SMILES: Cn1nc(N2CCC(=O)NC2=O)c2cc(F)c(C3CCN(CC4CCN(c5cc(S(=O)(=O)N6CCC(Nc7ncc(Cl)cn7)CC6)ccc5C#N)CC4)CC3)cc21] | 4-((4-((5-chloropyrimidin- 2-yl)amino)piperidin-1- yl)sulfonyl)-2-(4-((4-(3- (2,4- dioxotetrahydropyrimidin- 1(2H)-yl)-5-fluoro-1- methyl-1H-indazol-6- yl)piperidin-1- yl)methyl)piperidin-1- yl)benzonitrile | 818.1 |
| 154 | 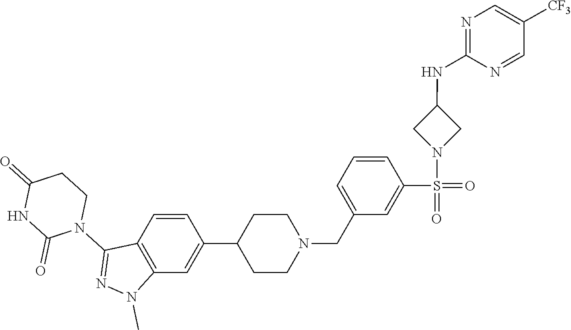 [SMILES: Cn1nc(N2CCC(=O)NC2=O)c2ccc(C3CCN(Cc4cccc(S(=O)(=O)N5CC(Nc6ncc(C(F)(F)F)cn6)C5)c4)CC3)cc21] | 1-(1-methyl-6-(1-(3-((3-((5- (trifluoromethyl)pyrimidin- 2-yl)amino)azetidin-1- yl)sulfonyl)benzyl) piperidin-4-yl)- 1H-indazol-3- yl)dihydropyrimidine- 2,4(1H,3H)-dione | 698.0 |
| 155 | 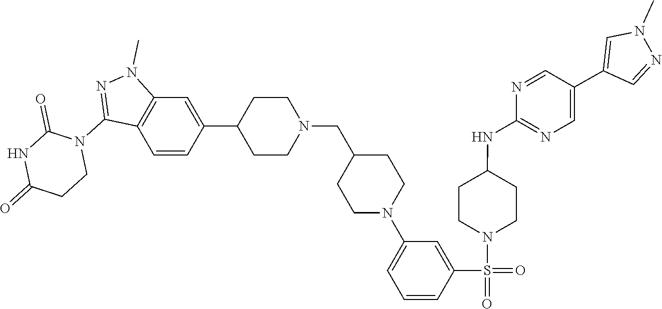 [SMILES: Cn1cc(-c2cnc(NC3CCN(S(=O)(=O)c4cccc(N5CCC(CN6CCC(c7ccc8c(N9CCC(=O)NC9=O)nn(C)c8c7)CC6)CC5)c4)CC3)nc2)cn1] | 1-(1-methyl-6-(1-((1-(3-((4- ((5-(1-methyl-1H-pyrazol- 4-yl)pyrimidin-2- yl)amino)piperidin-1- yl)sulfonyl)phenyl) piperidin-4-yl)methyl) piperidin-4- yl)-1H-indazol-3- yl)dihydropyrimidine- 2,4(1H,3H)-dione | 821.2 |
| 156 | 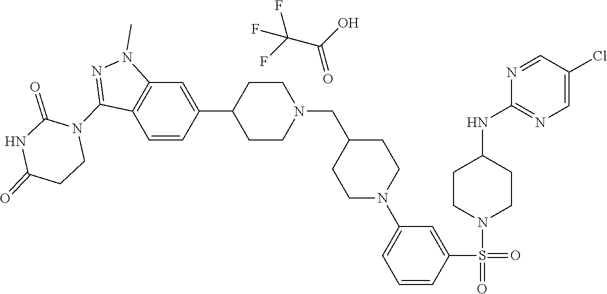 | 1-(6-(1-((4-(3-((4-((5- chloropyrimidin-2- yl)amino)piperidin-1- yl)sulfonyl)phenyl) cyclohexyl)methyl) piperidin-4-yl)-1- methyl-1H-indazol-3- yl)dihydropyrimidine- 2,4(1H,3H)-dione 2,2,2- trifluoroacetate | 774.1 |
| 157 | 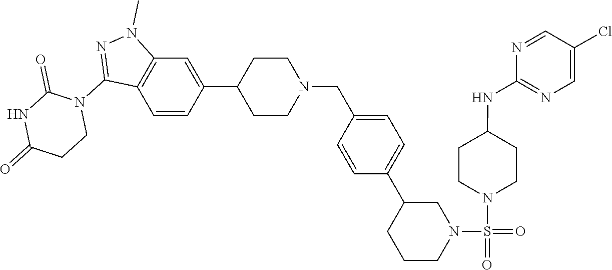 [SMILES: Cn1nc(N2CCC(=O)NC2=O)c2ccc(C3CCN(Cc4ccc(C5CCCN(S(=O)(=O)N6CCC(Nc7ncc(Cl)cn7)CC6)C5)cc4)CC3)cc21] | 1-(6-(1-(4-(1-((4-((5- chloropyrimidin-2- yl)amino)piperidin-1- yl)sulfonyl)piperidin-3- yl)benzyl)piperidin-4-yl)- 1-methyl-1H-indazol-3- yl)dihydropyrimidine- 2,4(1H,3H)-dione | 775.1 |
| 158 | 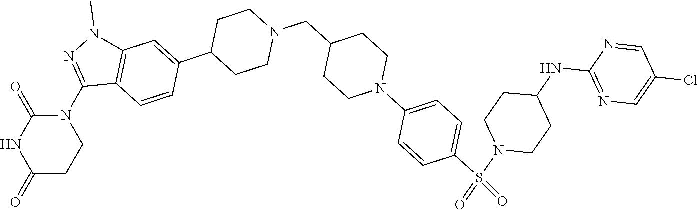 [SMILES: Cn1nc(N2CCC(=O)NC2=O)c2ccc(C3CCN(CC4CCN(c5ccc(S(=O)(=O)N6CCC(Nc7ncc(Cl)cn7)CC6)cc5)CC4)CC3)cc21] | 1-(6-(1-((1-(4-((4-((5- chloropyrimidin-2- yl)amino)piperidin-1- yl)sulfonyl)phenyl) piperidin-4-yl)methyl) piperidin-4- yl)-1-methyl-1H-indazol-3- yl)dihydropyrimidine- 2,4(1H,3H)-dione | 775.1 |
| 159 | 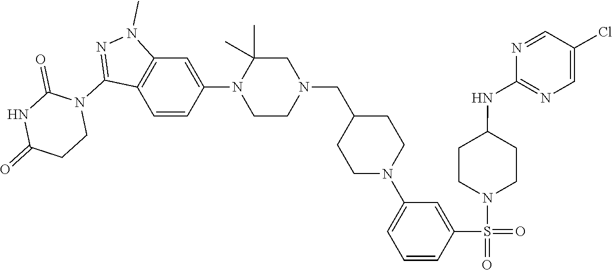 [SMILES: Cn1nc(N2CCC(=O)NC2=O)c2ccc(N3CCN(CC4CCN(c5cccc(S(=O)(=O)N6CCC(Nc7ncc(Cl)cn7)CC6)c5)CC4)CC3(C)C)cc21] | 1-(6-(4-((1-(3-((4-((5- chloropyrimidin-2- yl)amino)piperidin-1- yl)sulfonyl)phenyl) piperidin-4-yl) methyl)-2,2- dimethylpiperazin-1-yl)- 1-methyl-1H-indazol-3- yl)dihydropyrimidine- 2,4(1H,3H)-dione | 804.3 |
| 160 | 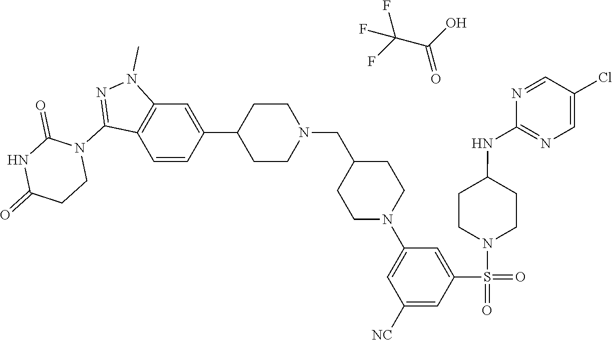 [SMILES: Cn1nc(N2CCC(=O)NC2=O)c2ccc(C3CCN(CC4CCN(c5cc(C#N)cc(S(=O)(=O)N6CCC(Nc7ncc(Cl)cn7)CC6)c5)CC4)CC3)cc21 ; O=C(O)C(F)(F)F] | 3-((4-((5-chloropyrimidin- 2-yl)amino)piperidin-1- yl)sulfonyl)-5-(4-((4-(3- (2,4- dioxotetrahydropyrimidin- 1(2H)-yl)-1-methyl-1H- indazol-6-yl)piperidin-1- yl)methyl)piperidin-1- yl)benzonitrile 2,2,2- trifluoroacetate | 800.2 |
| 161 | 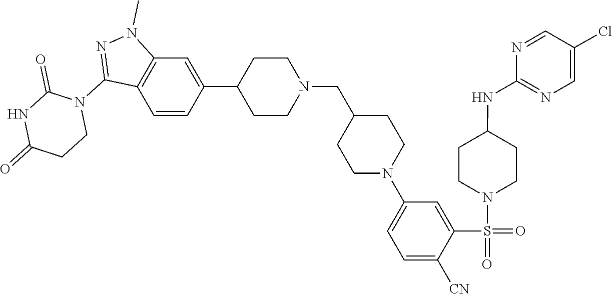 [SMILES: Cn1nc(N2CCC(=O)NC2=O)c2ccc(C3CCN(CC4CCN(c5ccc(C#N)c(S(=O)(=O)N6CCC(Nc7ncc(Cl)cn7)CC6)c5)CC4)CC3)cc21] | 2-((4-((5-chloropyrimidin- 2-yl)amino)piperidin-1- yl)sulfonyl)-4-(4-((4-(3- (2,4- dioxotetrahydropyrimidin- 1(2H)-yl)-1-methyl-1H- indazol-6-yl)piperidin-1- yl)methyl)piperidin-1- yl)benzonitrile | 800.2 |
| 162 | 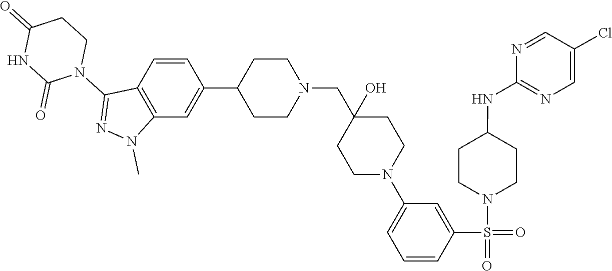 [SMILES: Cn1nc(N2CCC(=O)NC2=O)c2ccc(C3CCN(CC4(O)CCN(c5cccc(S(=O)(=O)N6CCC(Nc7ncc(Cl)cn7)CC6)c5)CC4)CC3)cc21] | 1-(6-(1-((1-(3-((4-((5- chloropyrimidin-2- yl)amino)piperidin-1- yl)sulfonyl)phenyl)-4- hydroxypiperidin-4- yl)methyl)piperidin-4-yl)- 1-methyl-1H-indazol-3- yl)dihydropyrimidine- 2,4(1H,3H)-dione | 791.1 |
| 163 | 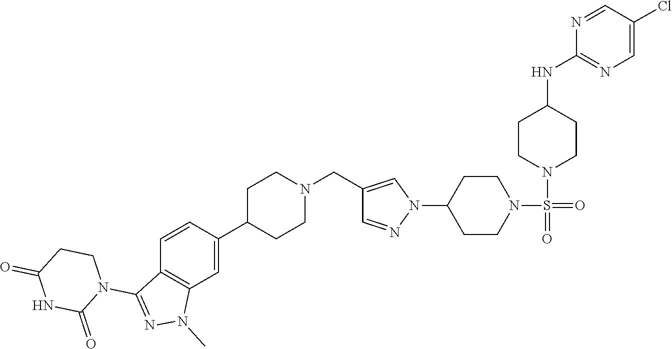 [SMILES: Cn1nc(N2CCC(=O)NC2=O)c2ccc(C3CCN(Cc4cnn(C5CCN(S(=O)(=O)N6CCC(Nc7ncc(Cl)cn7)CC6)CC5)c4)CC3)cc21] | 1-(6-(1-((1-(1-((4-((5- chloropyrimidin-2- yl)amino)piperidin-1- yl)sulfonyl)piperidin-4-yl)- 1H-pyrazol-4- yl)methyl)piperidin-4-yl)- 1-methyl-1H-indazol-3- yl)dihydropyrimidine- 2,4(1H,3H)-dione | 765.1 |
| 164 | 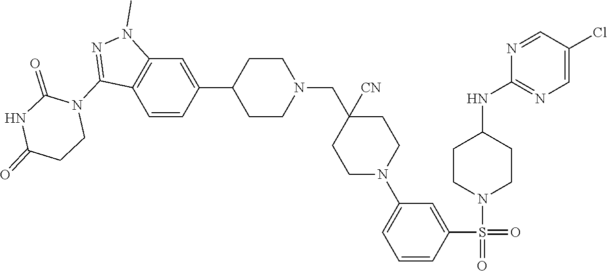 [SMILES: Cn1nc(N2CCC(=O)NC2=O)c2ccc(C3CCN(CC4(C#N)CCN(c5cccc(S(=O)(=O)N6CCC(Nc7ncc(Cl)cn7)CC6)c5)CC4)CC3)cc21] | 1-(3-((4-((5- chloropyrimidin-2- yl)amino)piperidin-1- yl)sulfonyl)phenyl)-4-((4- (3-(2,4- dioxotetrahydropyrimidin- 1(2H)-yl)-1-methyl-1H- indazol-6-yl)piperidin-1- yl)methyl)piperidine-4- carbonitrile | 800.1 |
| 165 | 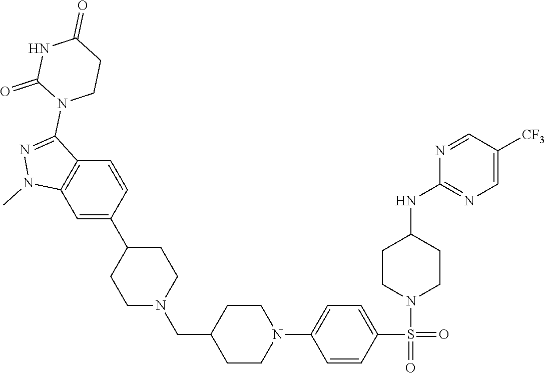 [SMILES: Cn1nc(N2CCC(=O)NC2=O)c2ccc(C3CCN(CC4CCN(c5ccc(S(=O)(=O)N6CCC(Nc7ncc(C(F)(F)F)cn7)CC6)cc5)CC4)CC3)cc21] | 1-(1-methyl-6-(1-((1-(4-((4- ((5- (trifluoromethyl)pyrimidin- 2-yl)amino)piperidin-1- yl)sulfonyl)phenyl) piperidin-4-yl)methyl) piperidin-4- yl)-1H-indazol-3- yl)dihydropyrimidine- 2,4(1H,3H)-dione | 809.1 |
| 166 | 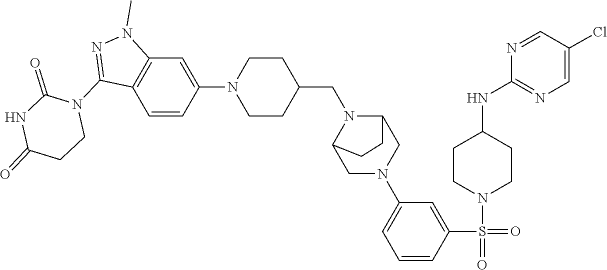 [SMILES: Cn1nc(N2CCC(=O)NC2=O)c2ccc(N3CCC(CN4C5CCC4CN(c4cccc(S(=O)(=O)N6CCC(Nc7ncc(Cl)cn7)CC6)c4)C5)CC3)cc21] | 1-(6-(4-((3-(3-((4-((5- chloropyrimidin-2- yl)amino)piperidin-1- yl)sulfonyl)phenyl)-3,8- diazabicyclo[3.2.1]octan-8- yl)methyl)piperidin-1-yl)-1- methyl-1H-indazol-3- yl)dihydropyrimidine- 2,4(1H,3H)-dione | 802.1 |
| 167 | 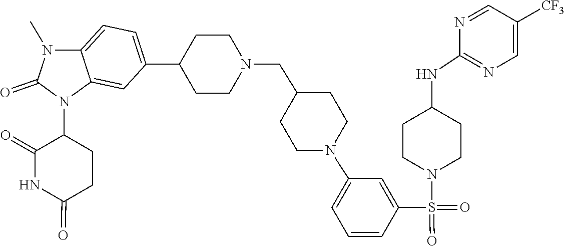 [SMILES: Cn1c(=O)n(C2CCC(=O)NC2=O)c2cc(C3CCN(CC4CCN(c5cccc(S(=O)(=O)N6CCC(Nc7ncc(C(F)(F)F)cn7)CC6)c5)CC4)CC3)ccc21] | 3-(3-methyl-2-oxo-6-(1-((1- (3-((4-((5- (trifluoromethyl)pyrimidin- 2-yl)amino)piperidin-1- yl)sulfonyl)phenyl) piperidin-4-yl)methyl) piperidin-4- yl)-2,3-dihydro-1H- benzo[d]imidazol-1- yl)piperidine-2,6-dione | 824.1 |
| 168 | 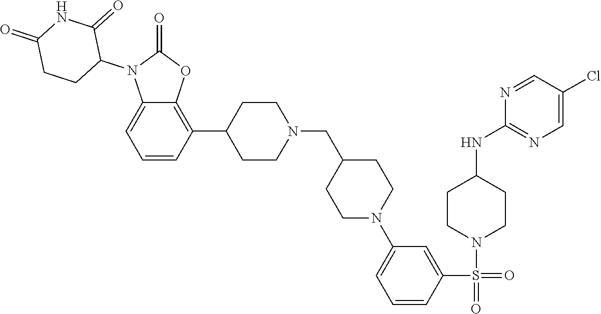 [SMILES: O=C1CCC(n2c(=O)oc3c(C4CCN(CC5CCN(c6cccc(S(=O)(=O)N7CCC(Nc8ncc(Cl)cn8)CC7)c6)CC5)CC4)cccc32)C(=O)N1] | 3-(7-(1-((1-(3-((4-((5- chloropyrimidin-2- yl)amino)piperidin-1- yl)sulfonyl)phenyl) piperidin-4-yl)methyl) piperidin-4- yl)-2-oxobenzo[d]oxazol- 3(2H)-yl)piperidine-2,6- dione | 777.1 |
| 169 | 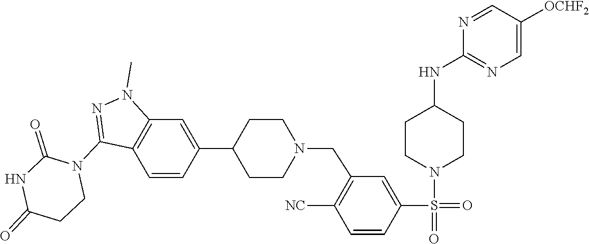 [SMILES: Cn1nc(N2CCC(=O)NC2=O)c2ccc(C3CCN(Cc4cc(S(=O)(=O)N5CCC(Nc6ncc(OC(F)F)cn6)CC5)ccc4C#N)CC3)cc21] | 4-((4-((5- (difluoromethoxy) pyrimidin-2-yl)amino) piperidin-1- yl)sulfonyl)-2-((4-(3-(2,4- dioxotetrahydropyrimidin- 1(2H)-yl)-1-methyl-1H- indazol-6-yl)piperidin-1- yl)methyl)benzonitrile | 749.1 |
| 170 | 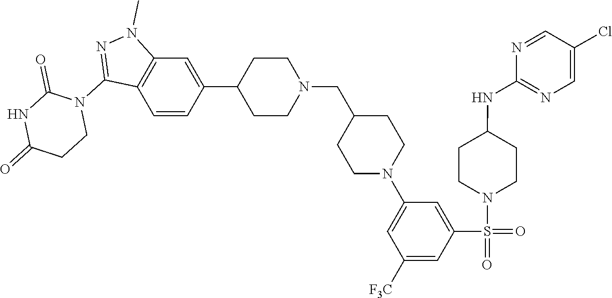 [SMILES: Cn1nc(N2CCC(=O)NC2=O)c2ccc(C3CCN(CC4CCN(c5cc(C(F)(F)F)cc(S(=O)(=O)N6CCC(Nc7ncc(Cl)cn7)CC6)c5)CC4)CC3)cc21] | 1-(6-(1-((1-(3-((4-((5- chloropyrimidin-2- yl)amino)piperidin-1- yl)sulfonyl)-5- (trifluoromethyl)phenyl) piperidin-4- yl)methyl)piperidin-4-yl)- 1-methyl-1H-indazol-3- yl)dihydropyrimidine- 2,4(1H,3H)-dione | 843.0 |
| 171 | 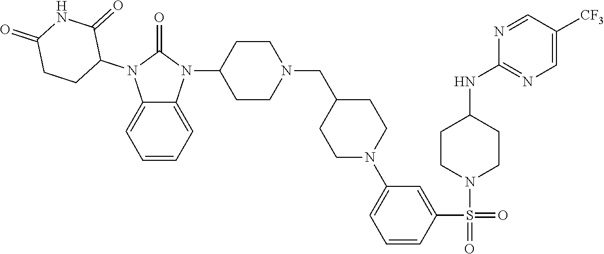 [SMILES: O=C1CCC(n2c(=O)n(C3CCN(CC4CCN(c5cccc(S(=O)(=O)N6CCC(Nc7ncc(C(F)(F)F)cn7)CC6)c5)CC4)CC3)c3ccccc32)C(=O)N1] | 3-(2-oxo-3-(1-((1-(3-((4- ((5- (trifluoromethyl)pyrimidin- 2-yl)amino)piperidin-1- yl)sulfonyl)phenyl) piperidin-4-yl)methyl) piperidin-4- yl)-2,3-dihydro-1H- benzo[d]imidazol-1- yl)piperidine-2,6-dione | 810.3 |
| 172 | 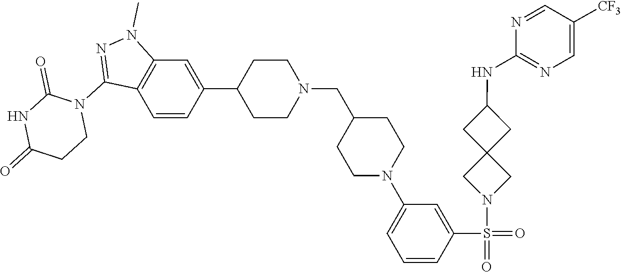 [SMILES: Cn1nc(N2CCC(=O)NC2=O)c2ccc(C3CCN(CC4CCN(c5cccc(S(=O)(=O)N6CC7(CC(Nc8ncc(C(F)(F)F)cn8)C7)C6)c5)CC4)CC3)cc21] | 1-(1-methyl-6-(1-((1-(3-((6- ((5- (trifluoromethyl)pyrimidin- 2-yl)amino)-2- azaspiro[3.3]heptan-2- yl)sulfonyl)phenyl) piperidin\-4-yl)methyl) piperidin-4- yl)-1H-indazol-3- yl)dihydropyrimidine- 2,4(1H,3H)-dione | 821.1 |
| 173 | 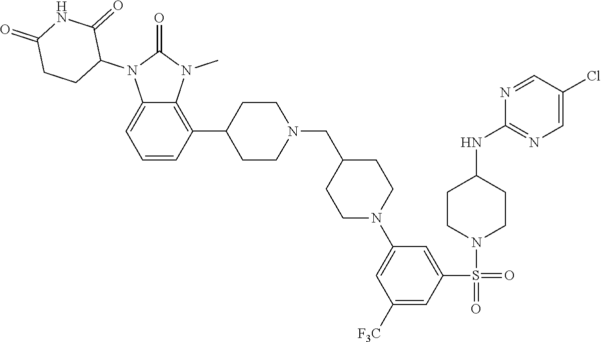 [SMILES: Cn1c(=O)n(C2CCC(=O)NC2=O)c2cccc(C3CCN(CC4CCN(c5cc(C(F)(F)F)cc(S(=O)(=O)N6CCC(Nc7ncc(Cl)cn7)CC6)c5)CC4)CC3)c21] | 3-(4-(1-((1-(3-((4-((5- chloropyrimidin-2- yl)amino)piperidin-1- yl)sulfonyl)-5- (trifluoromethyl)phenyl) piperidin-4- yl)methyl)piperidin-4-yl)-3- methyl-2-oxo-2,3-dihydro- 1H-benzo[d]imidazol-1- yl)piperidine-2,6-dione | 858.2 |
| 174 | 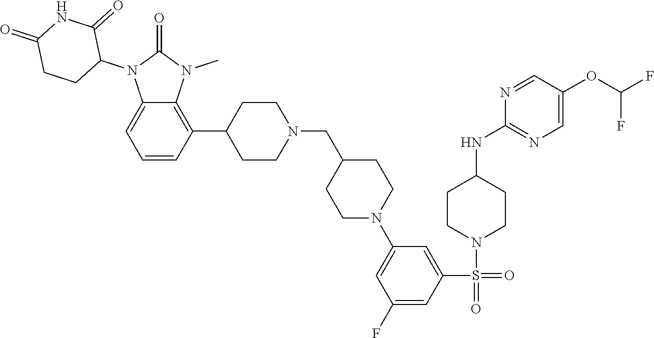 [SMILES: Cn1c(=O)n(C2CCC(=O)NC2=O)c2cccc(C3CCN(CC4CCN(c5cc(F)cc(S(=O)(=O)N6CCC(Nc7ncc(OC(F)F)cn7)CC6)c5)CC4)CC3)c21] | 3-(4-(1-((1-(3-((4-((5- (difluoromethoxy) pyrimidin-2-yl)amino) piperidin-1- yl)sulfonyl)-5- fluorophenyl)piperidin-4- yl)methyl)piperidin-4-yl)-3- methyl-2-oxo-2,3-dihydro- 1H-benzo[d]imidazol-1- yl)piperidine-2,6-dione | 840.1 |
| 175 | 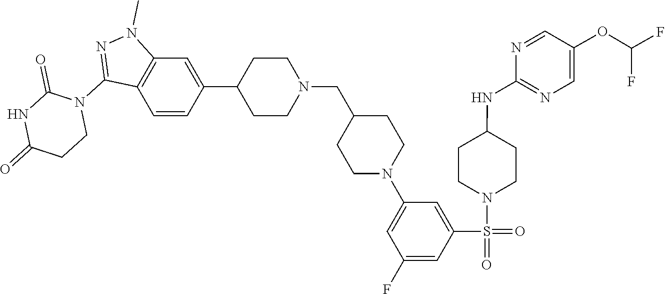 [SMILES: Cn1nc(N2CCC(=O)NC2=O)c2ccc(C3CCN(CC4CCN(c5cc(F)cc(S(=O)(=O)N6CCC(Nc7ncc(OC(F)F)cn7)CC6)c5)CC4)CC3)cc21] | 1-(6-(1-((1-(3-((4-((5- (difluoromethoxy) pyrimidin-2-yl)amino) piperidin-1- yl)sulfonyl)-5- fluorophenyl)piperidin-4- yl)methyl)piperidin-4-yl)-1- methyl-1H-indazol-3- yl)dihydropyrimidine- 2,4(1H,3H)-dione | 825.1 |
| 176 | 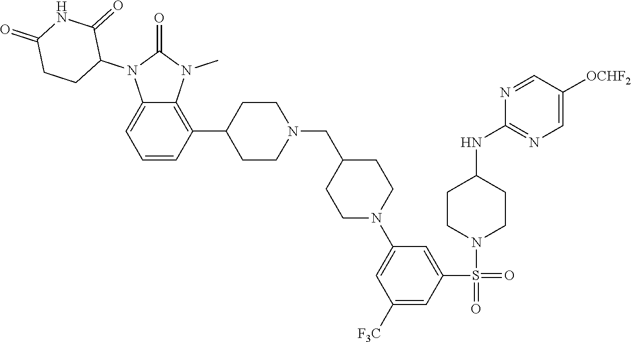 [SMILES: Cn1c(=O)n(C2CCC(=O)NC2=O)c2cccc(C3CCN(CC4CCN(c5cc(C(F)(F)F)cc(S(=O)(=O)N6CCC(Nc7ncc(OC(F)F)cn7)CC6)c5)CC4)CC3)c21] | 3-(4-(1-((1-(3-((4-((5- (difluoromethoxy) pyrimidin-2-yl)amino) piperidin-1- yl)sulfonyl)-5- (trifluoromethyl)phenyl) piperidin-4- yl)methyl)piperidin-4-yl)-3- methyl-2-oxo-2,3-dihydro- 1H-benzo[d]imidazol-1- yl)piperidine-2,6-dione | 890.2 |
| 177 | 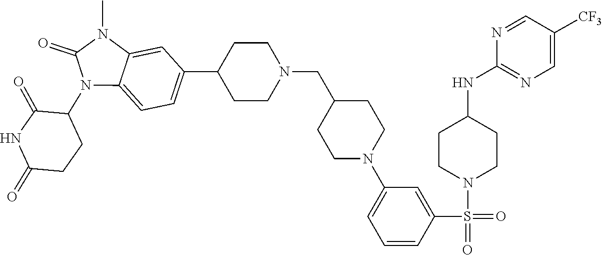 [SMILES: Cn1c(=O)n(C2CCC(=O)NC2=O)c2ccc(C3CCN(CC4CCN(c5cccc(S(=O)(=O)N6CCC(Nc7ncc(C(F)(F)F)cn7)CC6)c5)CC4)CC3)cc21] | 3-(3-methyl-2-oxo-5-(1-((1- (3-((4-((5- (trifluoromethyl)pyrimidin- 2-yl)amino)piperidin-1- yl)sulfonyl)phenyl) piperidin-4-yl)methyl) piperidin-4- yl)-2,3-dihydro-1H- benzo[d]imidazol-1- yl)piperidine-2,6-dione | 824.1 |
| 178 | 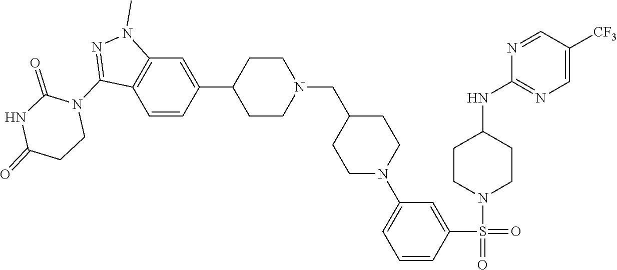 [SMILES: Cn1nc(N2CCC(=O)NC2=O)c2ccc(C3CCN(CC4CCN(c5cccc(S(=O)(=O)N6CCC(Nc7ncc(C(F)(F)F)cn7)CC6)c5)CC4)CC3)cc21] | 1-(1-methyl-6-(1-((1-(3-((3- ((5- (trifluoromethyl)pyrimidin- 2-yl)amino)azetidin-1- yl)sulfonyl)phenyl) piperidin-4-yl)methyl) piperidin-4- yl)-1H-indazol-3- yl)dihydropyrimidine- 2,4(1H,3H)-dione | 781.1 |
| 179 | 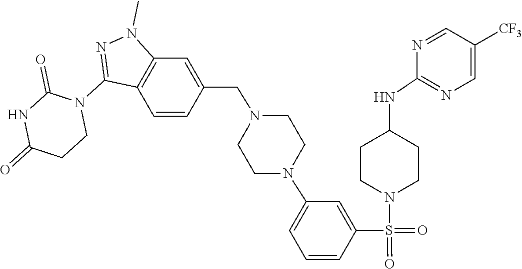 [SMILES: Cn1nc(N2CCC(=O)NC2=O)c2ccc(CN3CCN(c4cccc(S(=O)(=O)N5CCC(Nc6ncc(C(F)(F)F)cn6)CC5)c4)CC3)cc21] | 1-(1-methyl-6-((4-(3-((4- ((5- (trifluoromethyl)pyrimidin- 2-yl)amino)piperidin-1- yl)sulfonyl)phenyl) piperazin-1-yl)methyl)- 1H-indazol- 3-yl)dihydropyrimidine- 2,4(1H,3H)-dione | 727.2 |
| 180 | 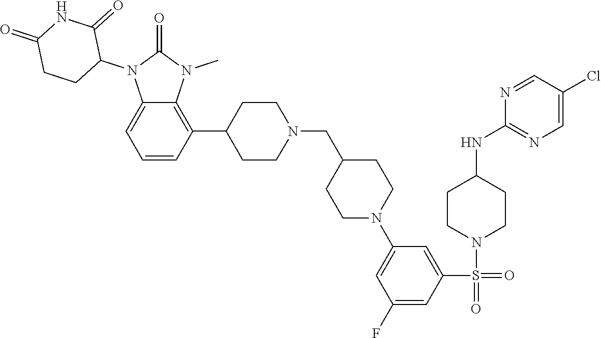 [SMILES: Cn1c(=O)n(C2CCC(=O)NC2=O)c2cccc(C3CCN(CC4CCN(c5cc(F)cc(S(=O)(=O)N6CCC(Nc7ncc(Cl)cn7)CC6)c5)CC4)CC3)c21] | 3-(4-(1-((1-(3-((4-((5- chloropyrimidin-2- yl)amino)piperidin-1- yl)sulfonyl)-5- fluorophenyl)piperidin-4- yl)methyl)piperidin-4-yl)-3- methyl-2-oxo-2,3-dihydro- 1H-benzo[d]imidazol-1- yl)piperidine-2,6-dione | 808.2 |
| 181 | 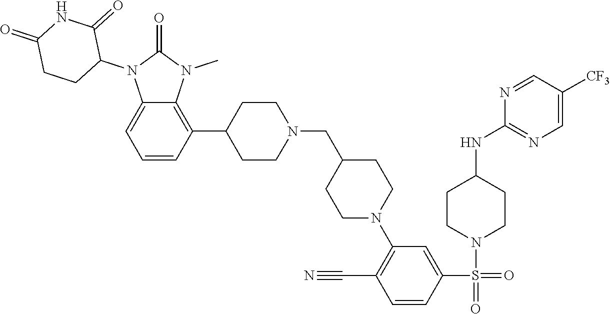 [SMILES: Cn1c(=O)n(C2CCC(=O)NC2=O)c2cccc(C3CCN(CC4CCN(c5cc(S(=O)(=O)N6CCC(Nc7ncc(C(F)(F)F)cn7)CC6)ccc5C#N)CC4)CC3)c21] | 2-(4-((4-(1-(2,6- dioxopiperidin-3-yl)-3- methyl-2-oxo-2,3-dihydro- 1H-benzo[d]imidazol-4- yl)piperidin-1- yl)methyl)piperidin-1- yl)-4-((4-((5- (trifluoromethyl)pyrimidin- 2-yl)amino)piperidin-1- yl)sulfonyl)benzonitrile | 849.5 |
| 182 | 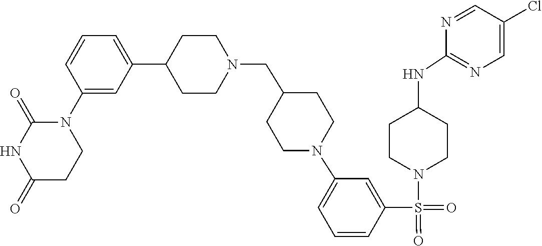 [SMILES: O=C1CCN(c2cccc(C3CCN(CC4CCN(c5cccc(S(=O)(=O)N6CCC(Nc7ncc(Cl)cn7)CC6)c5)CC4)CC3)c2)C(=O)N1] | 1-(3-(1-((1-(3-((4-((5- chloropyrimidin-2- yl)amino)piperidin-1- yl)sulfonyl)phenyl) piperidin-4-yl)methyl) piperidin-4-yl)phenyl) dihydropyrimidine- 2,4(1H,3H)-dione | 721.2 |
| 183 | 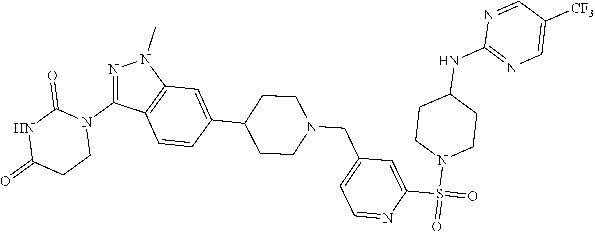 [SMILES: Cn1nc(N2CCC(=O)NC2=O)c2ccc(C3CCN(Cc4ccnc(S(=O)(=O)N5CCC(Nc6ncc(C(F)(F)F)cn6)CC5)c4)CC3)cc21] | 1-(1-methyl-6-(1-((2-((4- ((5- (trifluoromethyl)pyrimidin- 2-yl)amino)piperidin-1- yl)sulfonyl)pyridin-4- yl)methyl)piperidin-4-yl)- 1H-indazol-3- yl)dihydropyrimidine- 2,4(1H,3H)-dione | 727.1 |
| 184 | 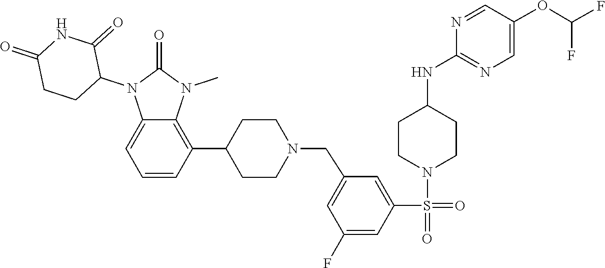 [SMILES: Cn1c(=O)n(C2CCC(=O)NC2=O)c2cccc(C3CCN(Cc4cc(F)cc(S(=O)(=O)N5CCC(Nc6ncc(OC(F)F)cn6)CC5)c4)CC3)c21] | 3-(4-(1-(3-((4-((5- (difluoromethoxy) pyrimidin-2-yl)amino) piperidin-1- yl)sulfonyl)-5- fluorobenzyl)piperidin-4- yl)-3-methyl-2-oxo-2,3- dihydro-1H- benzo[d]imidazol-1- yl)piperidine-2,6-dione | 757.1 |
| 185 | 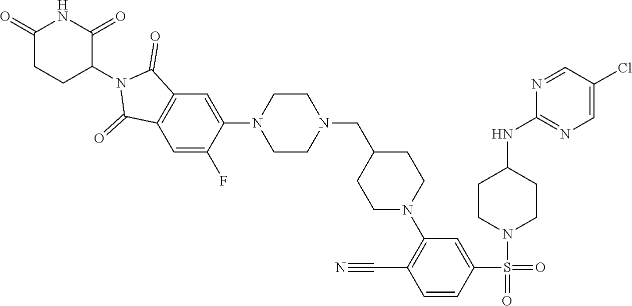 [SMILES: N#Cc1ccc(S(=O)(=O)N2CCC(Nc3ncc(Cl)cn3)CC2)cc1N1CCC(CN2CCN(c3cc4c(cc3F)C(=O)N(C3CCC(=O)NC3=O)C4=O)CC2)CC1] | 4-((4-((5-chloropyrimidin- 2-yl)amino)piperidin-1- yl)sulfonyl)-2-(4-((4-(2- (2,6-dioxopiperidin-3-yl)-6- fluoro-1,3-dioxoisoindolin- 5-yl)piperazin-1- yl)methyl)piperidin-1- yl)benzonitrile | 833.0 |
| 186 | 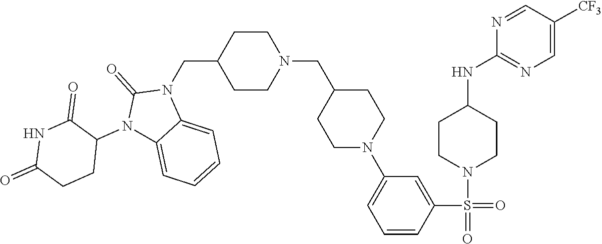 [SMILES: O=C1CCC(n2c(=O)n(CC3CCN(CC4CCN(c5cccc(S(=O)(=O)N6CCC(Nc7ncc(C(F)(F)F)cn7)CC6)c5)CC4)CC3)c3ccccc32)C(=O)N1] | 3-(2-oxo-3-((1-((1-(3-((4- ((5- (trifluoromethyl)pyrimidin- 2-yl)amino)piperidin-1- yl)sulfonyl)phenyl) piperidin-4-yl)methyl) piperidin-4- yl)methyl)-2,3-dihydro-1H- benzo[d]imidazol-1- yl)piperidine-2,6-dione | 824.5 |
| 187 | 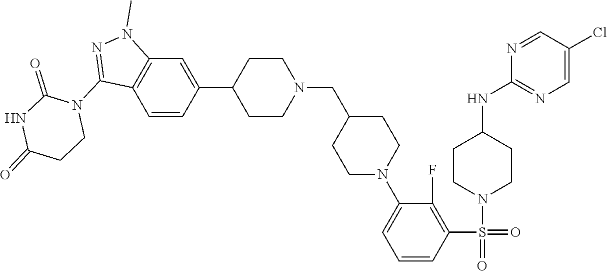 [SMILES: Cn1nc(N2CCC(=O)NC2=O)c2ccc(C3CCN(CC4CCN(c5cccc(S(=O)(=O)N6CCC(Nc7ncc(Cl)cn7)CC6)c5F)CC4)CC3)cc21] | 1-(6-(1-((1-(3-((4-((5- chloropyrimidin-2- yl)amino)piperidin-1- yl)sulfonyl)-2- fluorophenyl)piperidin-4- yl)methyl)piperidin-4-yl)-1- methyl-1H-indazol-3- yl)dihydropyrimidine- 2,4(1H,3H)-dione | 793.2 |
| 188 | 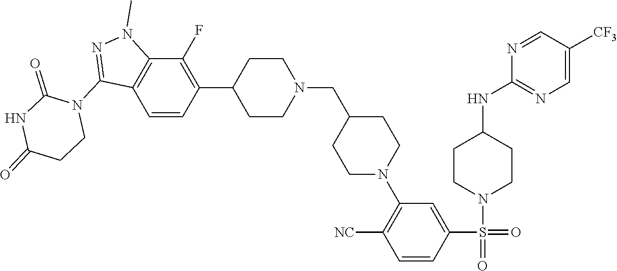 [SMILES: Cn1nc(N2CCC(=O)NC2=O)c2ccc(C3CCN(CC4CCN(c5cc(S(=O)(=O)N6CCC(Nc7ncc(C(F)(F)F)cn7)CC6)ccc5C#N)CC4)CC3)c(F)c21] | 2-(4-((4-(3-(2,4- dioxotetrahydropyrimidin- 1(2H)-yl)-7-fluoro-1- methyl-1H-indazol-6- yl)piperidin-1- yl)methyl)piperidin-1- yl)-4-((4-((5- (trifluoromethyl)pyrimidin- 2-yl)amino)piperidin-1- yl)sulfonyl)benzonitrile | 852.1 |
| 189 | 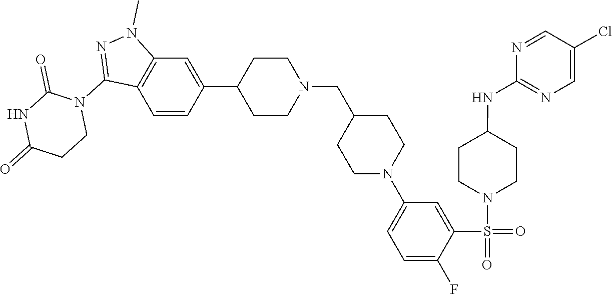 [SMILES: Cn1nc(N2CCC(=O)NC2=O)c2ccc(C3CCN(CC4CCN(c5ccc(F)c(S(=O)(=O)N6CCC(Nc7ncc(Cl)cn7)CC6)c5)CC4)CC3)cc21] | 1-(6-(1-((1-(3-((4-((5- chloropyrimidin-2- yl)amino)piperidin-1- yl)sulfonyl)-4- fluorophenyl)piperidin-4- yl)methyl)piperidin-4-yl)- 1-methyl-1H-indazol-3- yl)dihydropyrimidine- 2,4(1H,3H)-dione | 793.1 |
| 190 | 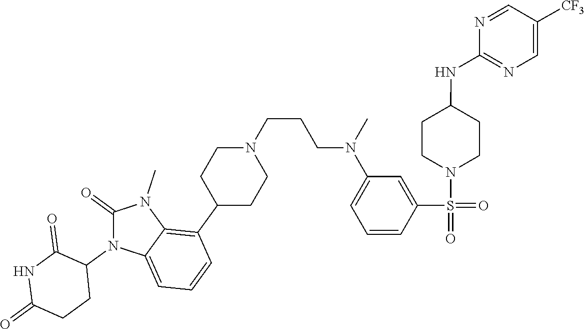 [SMILES: CN(CCCN1CCC(c2cccc3c2n(C)c(=O)n3C2CCC(=O)NC2=O)CC1)c1cccc(S(=O)(=O)N2CCC(Nc3ncc(C(F)(F)F)cn3)CC2)c1] | 3-(3-methyl-4-(1-(3- (methyl(3-((4-((5- (trifluoromethyl)pyrimidin- 2-yl)amino)piperidin-1- yl)sulfonyl)phenyl)amino) propyl)piperidin-4-yl)-2- oxo-2,3-dihydro-1H- benzo[d]imidazol-1- yl)piperidine-2,6-dione | 798.1 |
| 191 | 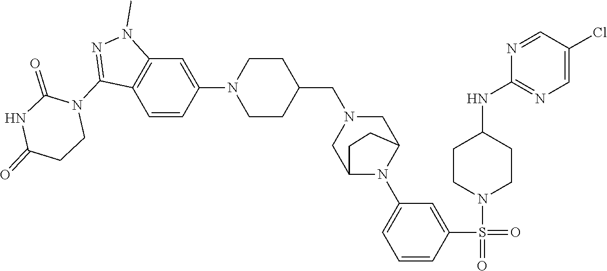 [SMILES: Cn1nc(N2CCC(=O)NC2=O)c2ccc(N3CCC(CN4CC5CCC(C4)N5c4cccc(S(=O)(=O)N5CCC(Nc6ncc(Cl)cn6)CC5)c4)CC3)cc21] | 1-(6-(4-((8-(3-((4-((5- chloropyrimidin-2- yl)amino)piperidin-1- yl)sulfonyl)phenyl)-3,8- diazabicyclo[3.2.1]octan-3- yl)methyl)piperidin-1-yl)- 1-methyl-1H-indazol-3- yl)dihydropyrimidine- 2,4(1H,3H)-dione | 802.1 |
| 192 | 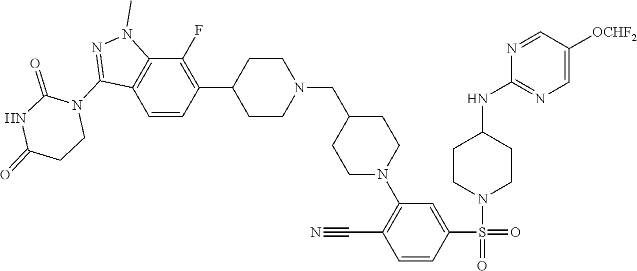 [SMILES: Cn1nc(N2CCC(=O)NC2=O)c2ccc(C3CCN(CC4CCN(c5cc(S(=O)(=O)N6CCC(Nc7ncc(OC(F)F)cn7)CC6)ccc5C#N)CC4)CC3)c(F)c21] | 4-((4-((5- (difluoromethoxy) pyrimidin-2-yl)amino) piperidin-1- yl)sulfonyl)-2-(4-((4-(3- (2,4- dioxotetrahydropyrimidin- 1(2H)-yl)-7-fluoro-1- methyl-1H-indazol-6- yl)piperidin-1- yl)methyl)piperidin-1- yl)benzonitrile | 850.1 |
| 193 | 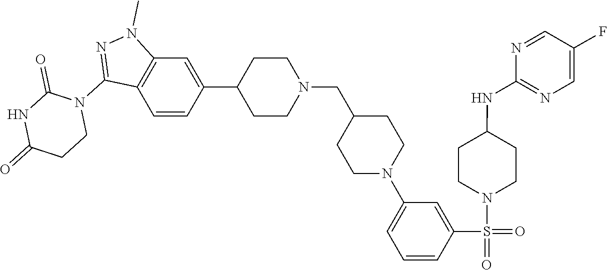 [SMILES: Cn1nc(N2CCC(=O)NC2=O)c2ccc(C3CCN(CC4CCN(c5cccc(S(=O)(=O)N6CCC(Nc7ncc(F)cn7)CC6)c5)CC4)CC3)cc21] | 1-(6-(1-((1-(3-((4-((5- fluoropyrimidin-2- yl)amino)piperidin-1- yl)sulfonyl)phenyl) piperidin-4-yl)methyl) piperidin-4- yl)-1-methyl-1H-indazol- 3-yl)dihydropyrimidine- 2,4(1H,3H)-dione | 759.1 |
| 194 | 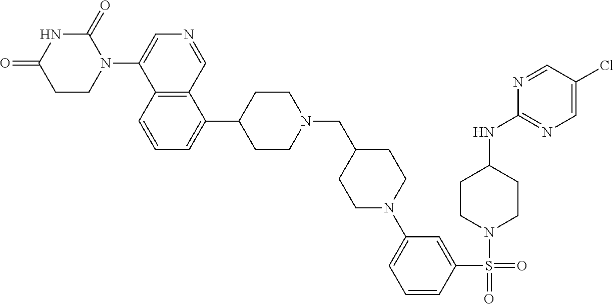 [SMILES: O=C1CCN(c2cncc3c(C4CCN(CC5CCN(c6cccc(S(=O)(=O)N7CCC(Nc8ncc(Cl)cn8)CC7)c6)CC5)CC4)cccc23)C(=O)N1] | 1-(8-(1-((1-(3-((4-((5- chloropyrimidin-2- yl)amino)piperidin-1- yl)sulfonyl)phenyl) piperidin-4-yl)methyl) piperidin-4- yl)isoquinolin-4- yl)dihydropyrimidine- 2,4(1H,3H)-dione | 772.1 |
| 195 | 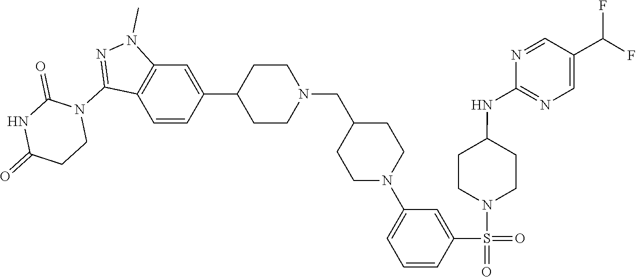 [SMILES: Cn1nc(N2CCC(=O)NC2=O)c2ccc(C3CCN(CC4CCN(c5cccc(S(=O)(=O)N6CCC(Nc7ncc(C(F)F)cn7)CC6)c5)CC4)CC3)cc21] | 1-(6-(1-((1-(3-((4-((5- (difluoromethyl)pyrimidin- 2-yl)amino)piperidin-1- yl)sulfonyl)phenyl) piperidin-4-yl)methyl) piperidin-4- yl)-1-methyl-1H-indazol- 3-yl)dihydropyrimidine- 2,4(1H,3H)-dione | 791.7 |
| 196 | 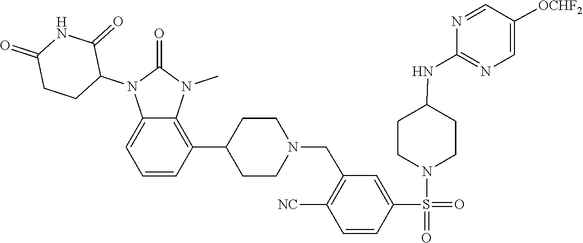 [SMILES: Cn1c(=O)n(C2CCC(=O)NC2=O)c2cccc(C3CCN(Cc4cc(S(=O)(=O)N5CCC(Nc6ncc(OC(F)F)cn6)CC5)ccc4C#N)CC3)c21] | 4-((4-((5- (difluoromethoxy) pyrimidin-2-yl)amino) piperidin-1- yl)sulfonyl)-2-((4-(1-(2,6- dioxopiperidin-3-yl)-3- methyl-2-oxo-2,3-dihydro- 1H-benzo[d]imidazol-4- yl)piperidin-1- yl)methyl)benzonitrile | 764.1 |
| 197 | 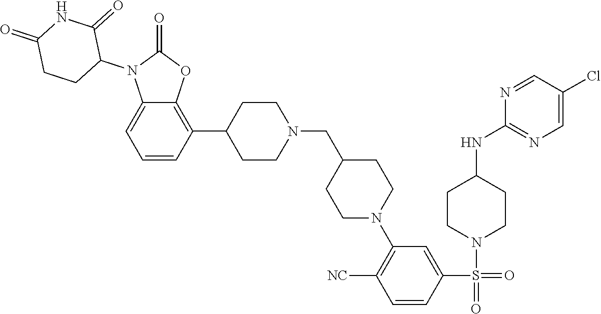 [SMILES: N#Cc1ccc(S(=O)(=O)N2CCC(Nc3ncc(Cl)cn3)CC2)cc1N1CCC(CN2CCC(c3cccc4c3oc(=O)n4C3CCC(=O)NC3=O)CC2)CC1] | 4-((4-((5-chloropyrimidin- 2-yl)amino)piperidin-1- yl)sulfonyl)-2-(4-((4-(3- (2,6-dioxopiperidin-3- yl)-2-oxo-2,3- dihydrobenzo[d]oxazol-7- yl)piperidin-1- yl)methyl)piperidin-1- yl)benzonitrile | 802.3 |
| 198 | 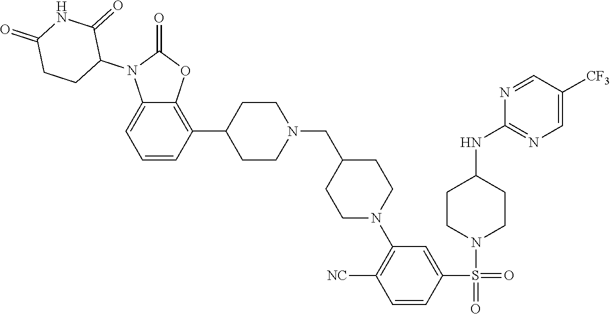 [SMILES: N#Cc1ccc(S(=O)(=O)N2CCC(Nc3ncc(C(F)(F)F)cn3)CC2)cc1N1CCC(CN2CCC(c3cccc4c3oc(=O)n4C3CCC(=O)NC3=O)CC2)CC1] | 2-(4-((4-(3-(2,6- dioxopiperidin-3-yl)-2-oxo- 2,3-dihydrobenzo[d]oxazol- 7-yl)piperidin-1- yl)methyl)piperidin-1- yl)-4-((4-((5- (trifluoromethyl)pyrimidin- 2-yl)amino)piperidin-1- yl)sulfonyl)benzonitrile | 836.6 |
| 199 | 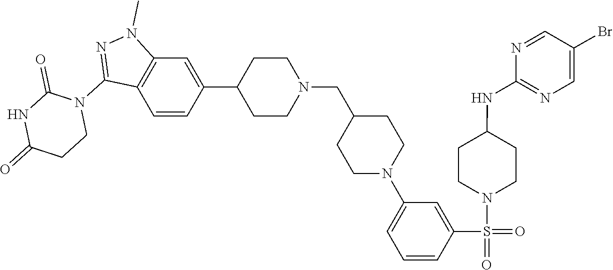 [SMILES: Cn1nc(N2CCC(=O)NC2=O)c2ccc(C3CCN(CC4CCN(c5cccc(S(=O)(=O)N6CCC(Nc7ncc(Br)cn7)CC6)c5)CC4)CC3)cc21] | 1-(6-(1-((1-(3-((4-((5- bromopyrimidin-2- yl)amino)piperidin-1- yl)sulfonyl)phenyl) piperidin-4-yl)methyl) piperidin-4- yl)-1-methyl-1H-indazol-3- yl)dihydropyrimidine- 2,4(1H,3H)-dione | 819.3 |
| 200 | 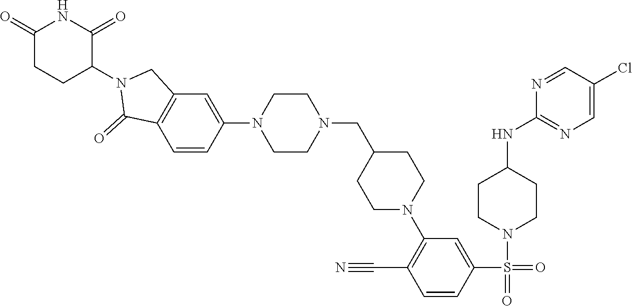 [SMILES: N#Cc1ccc(S(=O)(=O)N2CCC(Nc3ncc(Cl)cn3)CC2)cc1N1CCC(CN2CCN(c3ccc4c(c3)CN(C3CCC(=O)NC3=O)C4=O)CC2)CC1] | 4-((4-((5-chloropyrimidin- 2-yl)amino)piperidin-1- yl)sulfonyl)-2-(4-((4-(2- (2,6-dioxopiperidin-3-yl)- 1-oxoisoindolin-5- yl)piperazin-1- yl)methyl)piperidin-1- yl)benzonitrile | 801.0 |
| 201 | 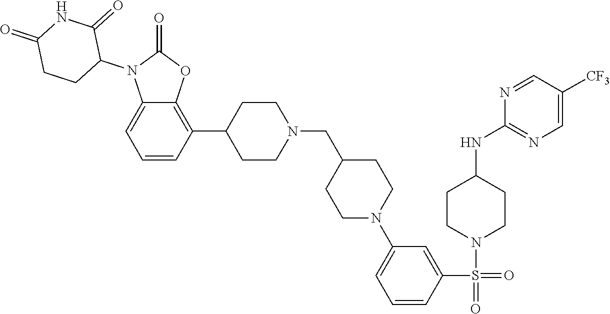 [SMILES: O=C1CCC(n2c(=O)oc3c(C4CCN(CC5CCN(c6cccc(S(=O)(=O)N7CCC(Nc8ncc(C(F)(F)F)cn8)CC7)c6)CC5)CC4)cccc32)C(=O)N1] | 3-(2-oxo-7-(1-((1-(3-((4- ((5- (trifluoromethyl)pyrimidin- 2-yl)amino)piperidin-1- yl)sulfonyl)phenyl) piperidin-4-yl)methyl) piperidin-4- yl)benzo[d]oxazol-3(2H)- yl)piperidine-2,6-dione | 811.6 |
| 202 | 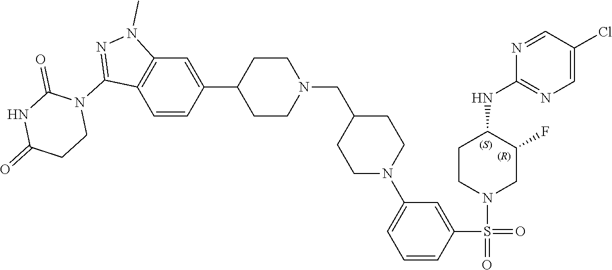 | Rac-1-(6-(1-((1-(3- (((3R,4S)-4-((5- chloropyrimidin-2- yl)amino)-3- fluoropiperidin-1- yl)sulfonyl)phenyl) piperidin-4-yl)methyl) piperidin-4- yl)-1-methyl-1H-indazol- 3-yl)dihydropyrimidine- 2,4(1H,3H)-dione | 793.2 |
| 203 | 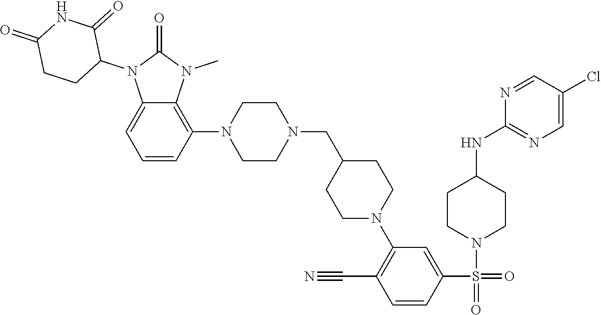 [SMILES: Cn1c(=O)n(C2CCC(=O)NC2=O)c2cccc(N3CCN(CC4CCN(c5cc(S(=O)(=O)N6CCC(Nc7ncc(Cl)cn7)CC6)ccc5C#N)CC4)CC3)c21] | 4-((4-((5-chloropyrimidin- 2-yl)amino)piperidin-1- yl)sulfonyl)-2-(4-((4-(1- (2,6-dioxopiperidin-3-yl)-3- methyl-2-oxo-2,3-dihydro- 1H-benzo[d]imidazol-4- yl)piperazin-1- yl)methyl)piperidin-1- yl)benzonitrile | 816.4 |
| 204 | 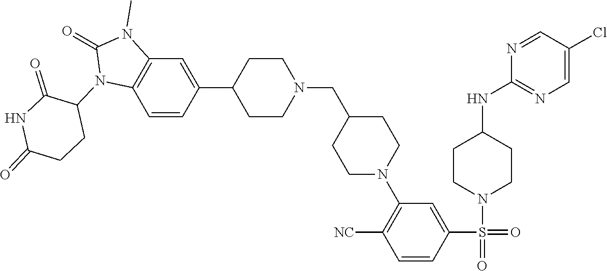 [SMILES: Cn1c(=O)n(C2CCC(=O)NC2=O)c2ccc(C3CCN(CC4CCN(c5cc(S(=O)(=O)N6CCC(Nc7ncc(Cl)cn7)CC6)ccc5C#N)CC4)CC3)cc21] | 4-((4-((5-chloropyrimidin- 2-yl)amino)piperidin-1- yl)sulfonyl)-2-(4-((4-(1- (2,6-dioxopiperidin-3-yl)-3- methyl-2-oxo-2,3-dihydro- 1H-benzo[d]imidazol-5- yl)piperidin-1- yl)methyl)piperidin-1- yl)benzonitrile | 815.4 |
| 205 | 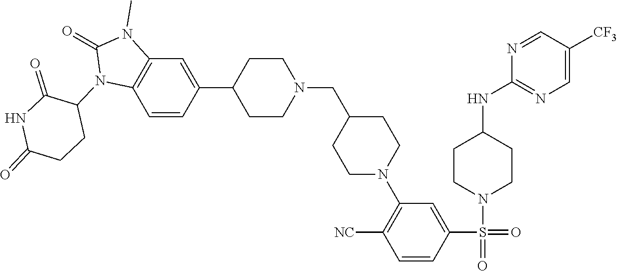 [SMILES: Cn1c(=O)n(C2CCC(=O)NC2=O)c2ccc(C3CCN(CC4CCN(c5cc(S(=O)(=O)N6CCC(Nc7ncc(C(F)(F)F)cn7)CC6)ccc5C#N)CC4)CC3)cc21] | 2-(4-((4-(1-(2,6- dioxopiperidin-3-yl)-3- methyl-2-oxo-2,3-dihydro- 1H-benzo[d]imidazol-5- yl)piperidin-1- yl)methyl)piperidin-1- yl)-4-((4-((5- (trifluoromethyl)pyrimidin- 2-yl)amino)piperidin-1- yl)sulfonyl)benzonitrile | 849.6 |
| 206 | 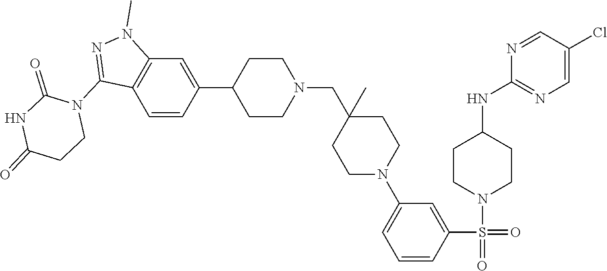 [SMILES: Cn1nc(N2CCC(=O)NC2=O)c2ccc(C3CCN(CC4(C)CCN(c5cccc(S(=O)(=O)N6CCC(Nc7ncc(Cl)cn7)CC6)c5)CC4)CC3)cc21] | 1-(6-(1-((1-(3-((4-((5- chloropyrimidin-2- yl)amino)piperidin-1- yl)sulfonyl)phenyl)-4- methylpiperidin-4- yl)methyl)piperidin-4-yl)- 1-methyl-1H-indazol-3- yl)dihydropyrimidine- 2,4(1H,3H)-dione | 789.1 |
| 207 | 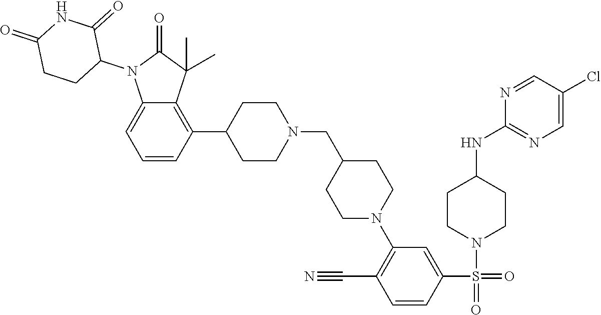 [SMILES: CC1(C)C(=O)N(C2CCC(=O)NC2=O)c2cccc(C3CCN(CC4CCN(c5cc(S(=O)(=O)N6CCC(Nc7ncc(Cl)cn7)CC6)ccc5C#N)CC4)CC3)c21] | 4-((4-((5-chloropyrimidin- 2-yl)amino)piperidin-1- yl)sulfonyl)-2-(4-((4-(1- (2,6-dioxopiperidin-3-yl)- 3,3-dimethyl-2-oxoindolin- 4-yl)piperidin-1- yl)methyl)piperidin-1- yl)benzonitrile | 828.1 |
| 208 | 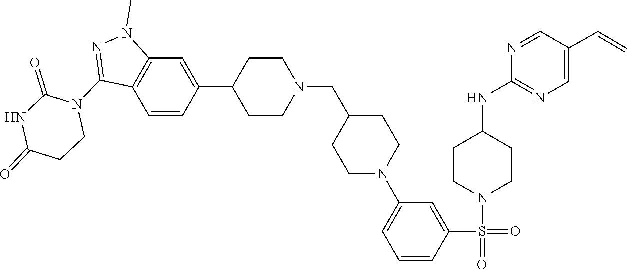 [SMILES: C=Cc1cnc(NC2CCN(S(=O)(=O)c3cccc(N4CCC(CN5CCC(c6ccc7c(N8CCC(=O)NC8=O)nn(C)c7c6)CC5)CC4)c3)CC2)nc1] | 1-(1-methyl-6-(1-((1-(3-((4- ((5-vinylpyrimidin-2- yl)amino)piperidin-1- yl)sulfonyl)phenyl) piperidin-4-yl)methyl) piperidin-4- yl)-1H-indazol-3- yl)dihydropyrimidine- 2,4(1H,3H)-dione | 767.2 |
| 209 | 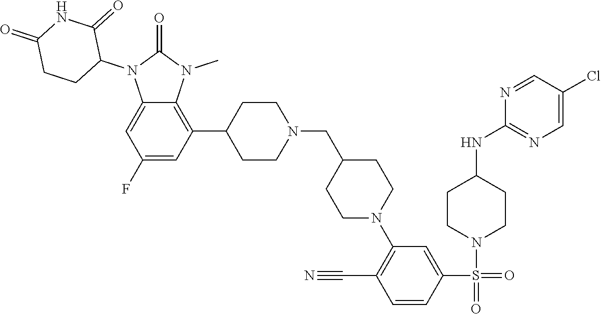 [SMILES: Cn1c(=O)n(C2CCC(=O)NC2=O)c2cc(F)cc(C3CCN(CC4CCN(c5cc(S(=O)(=O)N6CCC(Nc7ncc(Cl)cn7)CC6)ccc5C#N)CC4)CC3)c21] | 4-((4-((5-chloropyrimidin- 2-yl)amino)piperidin-1- yl)sulfonyl)-2-(4-((4-(1- (2,6-dioxopiperidin-3-yl)-6- fluoro-3-methyl-2-oxo-2,3- dihydro-1H- benzo[d]imidazol-4- yl)piperidin-1- yl)methyl)piperidin-1- yl)benzonitrile | 833.1 |
| 210 | 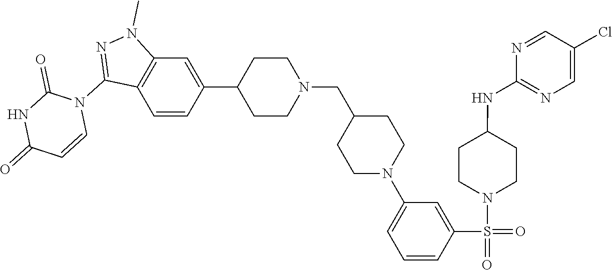 [SMILES: Cn1nc(-n2ccc(=O)[nH]c2=O)c2ccc(C3CCN(CC4CCN(c5cccc(S(=O)(=O)N6CCC(Nc7ncc(Cl)cn7)CC6)c5)CC4)CC3)cc21] | 1-(6-(1-((1-(3-((4-((5- chloropyrimidin-2- yl)amino)piperidin-1- yl)sulfonyl)phenyl) piperidin-4-yl)methyl) piperidin-4- yl)-1-methyl-1H-indazol-3- yl)pyrimidine-2,4(1H,3H)- dione | 773.4 |
| 211 | 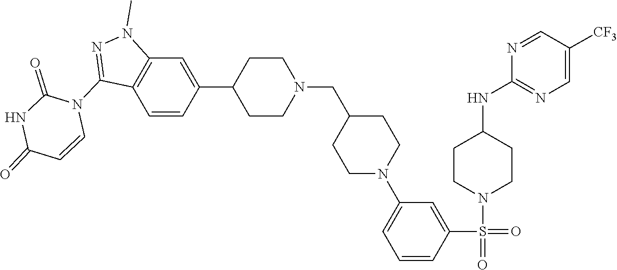 [SMILES: Cn1nc(-n2ccc(=O)[nH]c2=O)c2ccc(C3CCN(CC4CCN(c5cccc(S(=O)(=O)N6CCC(Nc7ncc(C(F)(F)F)cn7)CC6)c5)CC4)CC3)cc21] | 1-(1-methyl-6-(1-((1-(3-((4- ((5- (trifluoromethyl)pyrimidin- 2-yl)amino)piperidin-1- yl)sulfonyl)phenyl) piperidin-4-yl)methyl) piperidin-4- yl)-1H-indazol-3- yl)pyrimidine-2,4(1H,3H)- dione | 807.4 |
| 212 | 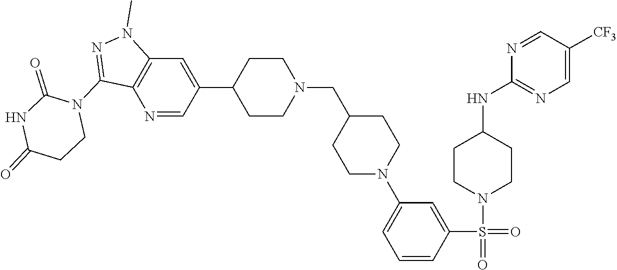 [SMILES: Cn1nc(N2CCC(=O)NC2=O)c2ncc(C3CCN(CC4CCN(c5cccc(S(=O)(=O)N6CCC(Nc7ncc(C(F)(F)F)cn7)CC6)c5)CC4)CC3)cc21] | 1-(1-methyl-6-(1-((1-(3-((4- ((5- (trifluoromethyl)pyrimidin- 2-yl)amino)piperidin-1- yl)sulfonyl)phenyl) piperidin-4-yl)methyl) piperidin-4- yl)-1H-pyrazolo[4,3- b]pyridin-3- yl)dihydropyrimidine- 2,4(1H,3H)-dione | 810.5 |
| 213 | 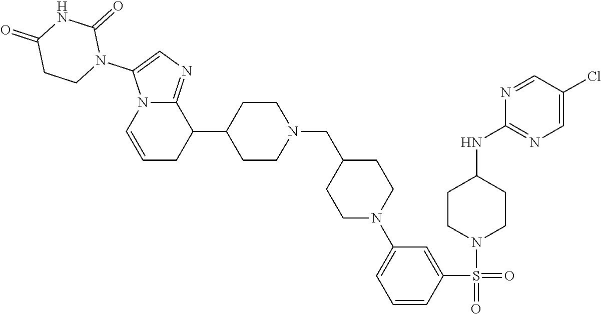 [SMILES: O=C1CCN(c2cnc3n2C=CCC3C2CCN(CC3CCN(c4cccc(S(=O)(=O)N5CCC(Nc6ncc(Cl)cn6)CC5)c4)CC3)CC2)C(=O)N1] | 1-(8-(1-((1-(3-((4-((5- chloropyrimidin-2- yl)amino)piperidin-1- yl)sulfonyl)phenyl) piperidin-4-yl)methyl) piperidin-4- yl)imidazo[1,2-a]pyridin- 3-yl)dihydropyrimidine- 2,4(1H,3H)-dione | 761.5 |
| 214 | 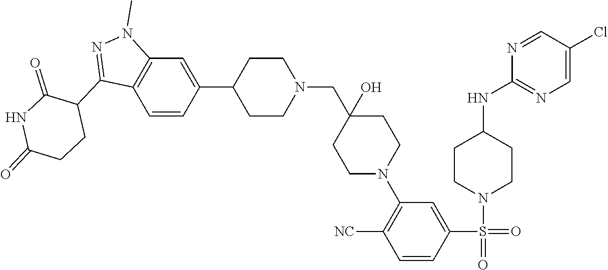 [SMILES: Cn1nc(C2CCC(=O)NC2=O)c2ccc(C3CCN(CC4(O)CCN(c5cc(S(=O)(=O)N6CCC(Nc7ncc(Cl)cn7)CC6)ccc5C#N)CC4)CC3)cc21] | 4-((4-((5-chloropyrimidin- 2-yl)amino)piperidin-1- yl)sulfonyl)-2-(4-((4-(3- (2,4- dioxotetrahydropyrimidin- 1(2H)-yl)-1-methyl-1H- indazol-6-yl)piperidin-1- yl)methyl)-4- hydroxypiperidin-1- yl)benzonitrile | 816.4 |
| 215 | 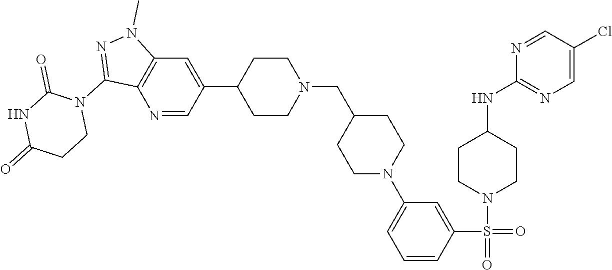 [SMILES: Cn1nc(N2CCC(=O)NC2=O)c2ncc(C3CCN(CC4CCN(c5cccc(S(=O)(=O)N6CCC(Nc7ncc(Cl)cn7)CC6)c5)CC4)CC3)cc21] | 1-(6-(1-((1-(3-((4-((5- 4,3-b]pyridin-3- yl)dihydropyrimidine- ] | 776.3 |
| 216 | 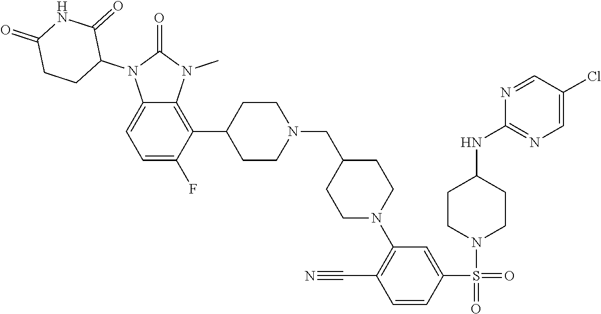 [SMILES: Cn1c(=O)n(C2CCC(=O)NC2=O)c2ccc(F)c(C3CCN(CC4CCN(c5cc(S(=O)(=O)N6CCC(Nc7ncc(Cl)cn7)CC6)ccc5C#N)CC4)CC3)c21] | 4-((4-((5-chloropyrimidin- 2-yl)amino)piperidin-1- yl)sulfonyl)-2-(4-((4-(1- (2,6-dioxopiperidin-3-yl)-5- fluoro-3-methyl-2-oxo-2,3- dihydro-1H- benzo[d]imidazol-4- yl)piperidin-1- yl)methyl)piperidin-1- yl)benzonitrile | 833.5 |
| 217 | 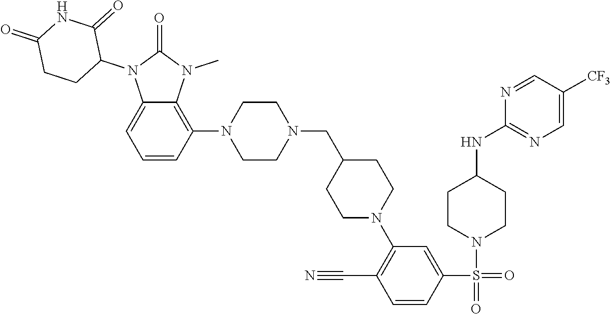 [SMILES: Cn1c(=O)n(C2CCC(=O)NC2=O)c2cccc(N3CCN(CC4CCN(c5cc(S(=O)(=O)N6CCC(Nc7ncc(C(F)(F)F)cn7)CC6)ccc5C#N)CC4)CC3)c21] | 2-(4-((4-(1-(2,6- dioxopiperidin-3-yl)-3- methyl-2-oxo-2,3-dihydro- 1H-benzo[d]imidazol-4- yl)piperazin-1- yl)methyl)piperidin-1-yl)-4- ((4-((5- (trifluoromethyl)pyrimidin- 2-yl)amino)piperidin-1- yl)sulfonyl)benzonitrile | 850.1 |
| 218 | 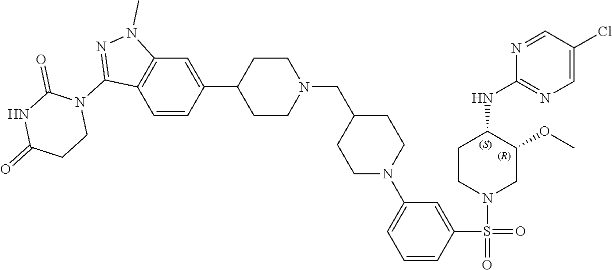 | 1-(6-(1-((1-(3-(((3R,4S)-4- ((5-chloropyrimidin-2- yl)amino)-3- methoxypiperidin-1- yl)sulfonyl)phenyl) piperidin-4-yl)methyl) piperidin-4- yl)-1-methyl-1H-indazol-3- yl)dihydropyrimidine- 2,4(1H,3H)-dione | 805.1 |
| 219 | 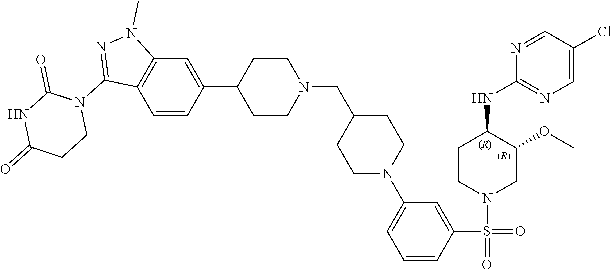 | Rac-1-(6-(1-((1-(3- (((3R,4R)-4-((5- chloropyrimidin-2- yl)amino)-3- methoxypiperidin-1- yl)sulfonyl)phenyl) piperidin-4-yl)methyl) piperidin-4- yl)-1-methyl-1H-indazol-3- yl)dihydropyrimidine- 2,4(1H,3H)-dione | 805.1 |
| 220 | 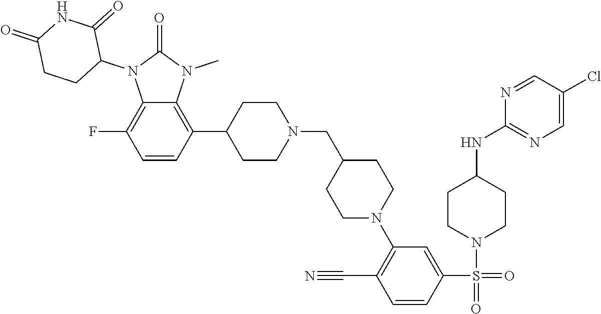 [SMILES: Cn1c(=O)n(C2CCC(=O)NC2=O)c2c(F)ccc(C3CCN(CC4CCN(c5cc(S(=O)(=O)N6CCC(Nc7ncc(Cl)cn7)CC6)ccc5C#N)CC4)CC3)c21] | 4-((4-((5-chloropyrimidin- 2-yl)amino)piperidin-1- yl)sulfonyl)-2-(4-((4-(1- (2,6-dioxopiperidin-3-yl)-7- fluoro-3-methyl-2-oxo-2,3- dihydro-1H- benzo[d]imidazol-4- yl)piperidin-1- yl)methyl)piperidin-1- yl)benzonitrile | 833.4 |
| 221 | 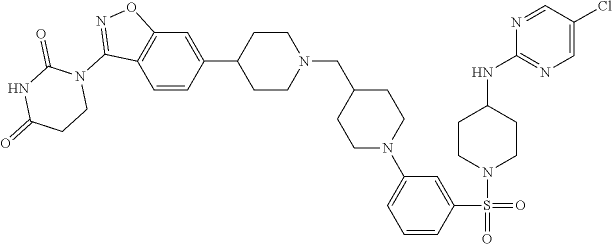 [SMILES: O=C1CCN(c2noc3cc(C4CCN(CC5CCN(c6cccc(S(=O)(=O)N7CCC(Nc8ncc(Cl)cn8)CC7)c6)CC5)CC4)ccc23)C(=O)N1] | 1-(6-(1-((1-(3-((4-((5- chloropyrimidin-2- yl)amino)piperidin-1- yl)sulfonyl)phenyl) piperidin-4-yl)methyl) piperidin-4- yl)benzo[d]isoxazol-3- yl)dihydropyrimidine- 2,4(1H,3H)-dione | 762.1 |
| 222 | 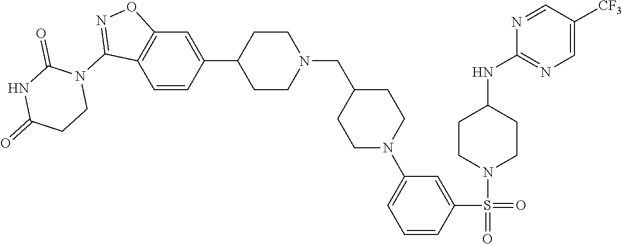 [SMILES: O=C1CCN(c2noc3cc(C4CCN(CC5CCN(c6cccc(S(=O)(=O)N7CCC(Nc8ncc(C(F)(F)F)cn8)CC7)c6)CC5)CC4)ccc23)C(=O)N1] | 1-(6-(1-((1-(3-((4-((5- (trifluoromethyl)pyrimidin- 2-yl)amino)piperidin-1- yl)sulfonyl)phenyl) piperidin-4-yl)methyl) piperidin-4- yl)benzo[d]isoxazol-3- yl)dihydropyrimidine- 2,4(1H,3H)-dione | 796.1 |
| 223 | 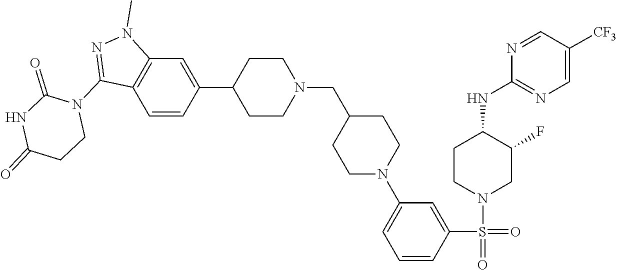 [SMILES: Cn1nc(N2CCC(=O)NC2=O)c2ccc(C3CCN(CC4CCN(c5cccc(S(=O)(=O)N6CC[C@H](Nc7ncc(C(F)(F)F)cn7)[C@H](F)C6)c5)CC4)CC3)cc21] | Rac-1-(6-(1-((1-(3- (((3R,4S)-3-fluoro-4-((5- (trifluoromethyl)pyrimidin- 2-yl)amino)piperidin-1- yl)sulfonyl)phenyl) piperidin-4-yl)methyl) piperidin-4- yl)-1-methyl-1H-indazol-3- yl)dihydropyrimidine- 2,4(1H,3H)-dione | 827.1 |
| 224 | 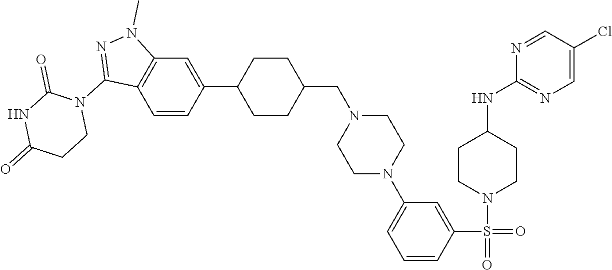 [SMILES: Cn1nc(N2CCC(=O)NC2=O)c2ccc(C3CCC(CN4CCN(c5cccc(S(=O)(=O)N6CCC(Nc7ncc(Cl)cn7)CC6)c5)CC4)CC3)cc21] | 1-(6-(4-((4-(3-((4-((5- chloropyrimidin-2- yl)amino)piperidin-1- yl)sulfonyl)phenyl) piperazin-1-yl)methyl) cyclohexyl)- 1-methyl-1H-indazol-3- yl)dihydropyrimidine- 2,4(1H,3H)-dione | 775.2 |
| 225 | 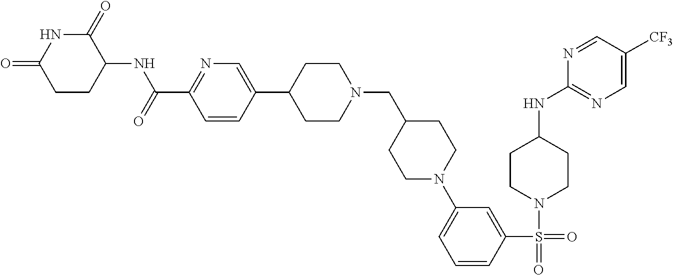 [SMILES: O=C1CCC(NC(=O)c2ccc(C3CCN(CC4CCN(c5cccc(S(=O)(=O)N6CCC(Nc7ncc(C(F)(F)F)cn7)CC6)c5)CC4)CC3)cn2)C(=O)N1] | N-(2,6-dioxopiperidin-3-yl)- 5-(1-((1-(3-((4-((5- (trifluoromethyl)pyrimidin- 2-yl)amino)piperidin-1- yl)sulfonyl)phenyl) piperidin-4-yl)methyl) piperidin-4- yl)picolinamide | 798.3 |
| 226 | 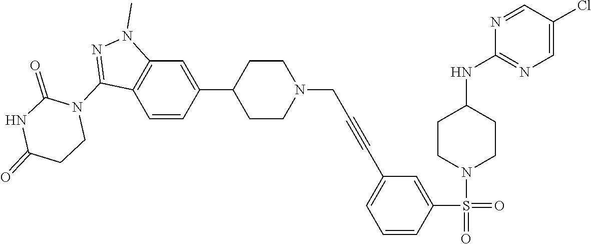 [SMILES: Cn1nc(N2CCC(=O)NC2=O)c2ccc(C3CCN(CC#Cc4cccc(S(=O)(=O)N5CCC(Nc6ncc(Cl)cn6)CC5)c4)CC3)cc21] | 1-(6-(1-(3-(3-((4-((5- chloropyrimidin-2- yl)amino)piperidin-1- yl)sulfonyl)phenyl)prop-2- yn-1-yl)piperidin-4-yl)-1- methyl-1H-indazol-3- yl)dihydropyrimidine- 2,4(1H,3H)-dione | 716.0 |
| 227 | 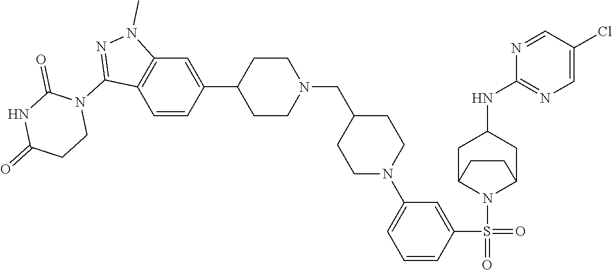 [SMILES: Cn1nc(N2CCC(=O)NC2=O)c2ccc(C3CCN(CC4CCN(c5cccc(S(=O)(=O)N6C7CCC6CC(Nc6ncc(Cl)cn6)C7)c5)CC4)CC3)cc21] | 1-(6-(1-((1-(3-(((1R,5S)-3- ((5-chloropyrimidin-2- yl)amino)-8- azabicyclo[3.2.1]octan-8- yl)sulfonyl)phenyl) piperidin-4-yl)methyl) piperidin-4- yl)-1-methyl-1H-indazol-3- yl)dihydropyrimidine- 2,4(1H,3H)-dione | 801.0 |
| 228 | 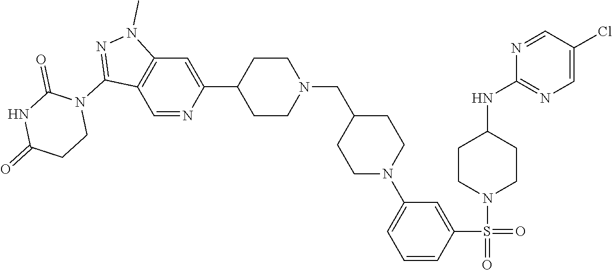 [SMILES: Cn1nc(N2CCC(=O)NC2=O)c2cnc(C3CCN(CC4CCN(c5cccc(S(=O)(=O)N6CCC(Nc7ncc(Cl)cn7)CC6)c5)CC4)CC3)cc21] | 1-(6-(1-((1-(3-((4-((5- chloropyrimidin-2- yl)amino)piperidin-1- yl)sulfonyl)phenyl) piperidin-4-yl)methyl) piperidin-4- yl)-1-methyl-1H- pyrazolo[4,3-c]pyridin-3- yl)dihydropyrimidine- 2,4(1H,3H)-dione | 776.0 |
| 229 | 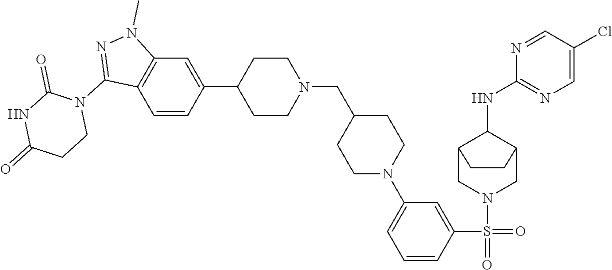 [SMILES: Cn1nc(N2CCC(=O)NC2=O)c2ccc(C3CCN(CC4CCN(c5cccc(S(=O)(=O)N6CC7CCC(C6)C7Nc6ncc(Cl)cn6)c5)CC4)CC3)cc21] | Rac-1-(6-(1-((1-(3- (((1R,5S,8s)-8-((5- chloropyrimidin-2- yl)amino)-3- azabicyclo[3.2.1]octan-3- yl)sulfonyl)phenyl) piperidin-4-yl)methyl) piperidin-4- yl)-1-methyl-1H-indazol-3- yl)dihydropyrimidine- 2,4(1H,3H)-dione | 801.1 |
| 230 | 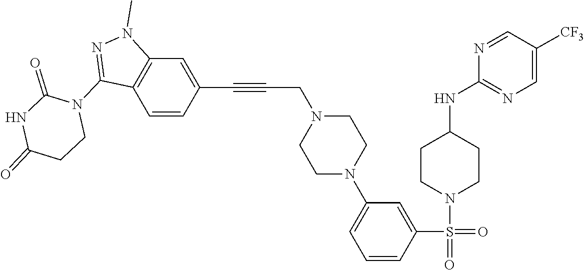 [SMILES: Cn1nc(N2CCC(=O)NC2=O)c2ccc(C#CCN3CCN(c4cccc(S(=O)(=O)N5CCC(Nc6ncc(C(F)(F)F)cn6)CC5)c4)CC3)cc21] | 1-(1-methyl-6-(3-(4-(3-((4- ((5- (trifluoromethyl)pyrimidin- 2-yl)amino)piperidin-1- yl)sulfonyl)phenyl) piperazin-1-yl)prop-1- yn-1-yl)-1H-indazol-3- yl)dihydropyrimidine- 2,4(1H,3H)-dione | 751.0 |
| 231 | 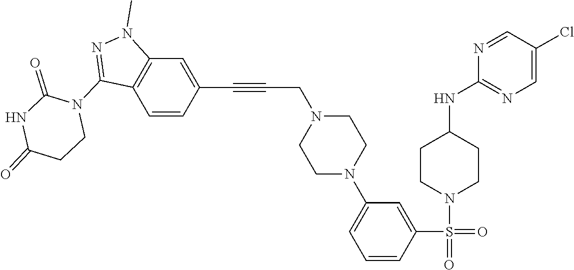 [SMILES: Cn1nc(N2CCC(=O)NC2=O)c2ccc(C#CCN3CCN(c4cccc(S(=O)(=O)N5CCC(Nc6ncc(Cl)cn6)CC5)c4)CC3)cc21] | 1-(6-(3-(4-(3-((4-((5- chloropyrimidin-2- yl)amino)piperidin-1- yl)sulfonyl)phenyl) piperazin-1-yl)prop- 1-yn-1-yl)-1- methyl-1H-indazol-3- yl)dihydropyrimidine- 2,4(1H,3H)-dione | 717.0 |
| 232 | 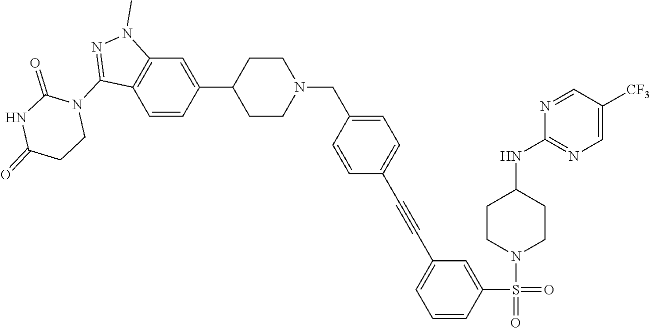 [SMILES: Cn1nc(N2CCC(=O)NC2=O)c2ccc(C3CCN(Cc4ccc(C#Cc5cccc(S(=O)(=O)N6CCC(Nc7ncc(C(F)(F)F)cn7)CC6)c5)cc4)CC3)cc21] | 1-(1-methyl-6-(1-(4-((3-((4- ((5- (trifluoromethyl)pyrimidin- 2-yl)amino)piperidin-1- yl)sulfonyl)phenyl)ethynyl) benzyl)piperidin-4-yl)-1H- indazol-3- yl)dihydropyrimidine- 2,4(1H,3H)-dione | 826.0 |
| 233 | 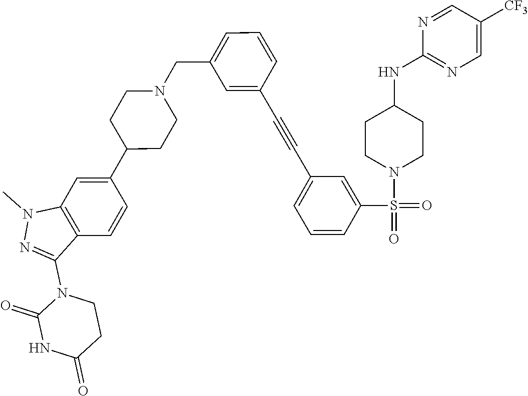 [SMILES: Cn1nc(N2CCC(=O)NC2=O)c2ccc(C3CCN(Cc4cccc(C#Cc5cccc(S(=O)(=O)N6CCC(Nc7ncc(C(F)(F)F)cn7)CC6)c5)c4)CC3)cc21] | 1-(6-(1-(4-((3-((4-((5- chloropyrimidin-2- yl)amino)piperidin-1- yl)sulfonyl)phenyl)ethynyl) benzyl)piperidin-4-yl)-1- methyl-1H-indazol-3- yl)dihydropyrimidine- 2,4(1H,3H)-dione | 826.0 |
| 234 | 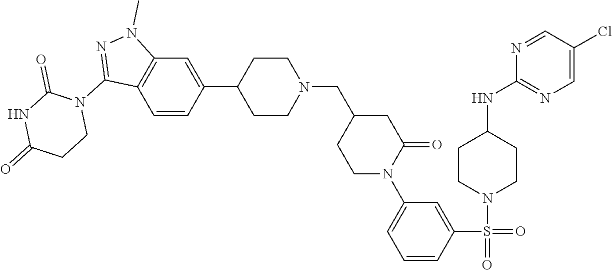 [SMILES: Cn1nc(N2CCC(=O)NC2=O)c2ccc(C3CCN(CC4CCN(c5cccc(S(=O)(=O)N6CCC(Nc7ncc(Cl)cn7)CC6)c5)C(=O)C4)CC3)cc21] | 1-(6-(1-((1-(3-((4-((5- chloropyrimidin-2- yl)amino)piperidin-1- yl)sulfonyl)phenyl)-2- oxopiperidin-4- yl)methyl)piperidin-4-yl)- 1-methyl-1H-indazol-3- yl)dihydropyrimidine- 2,4(1H,3H)-dione | 789.0 |
| 235 | 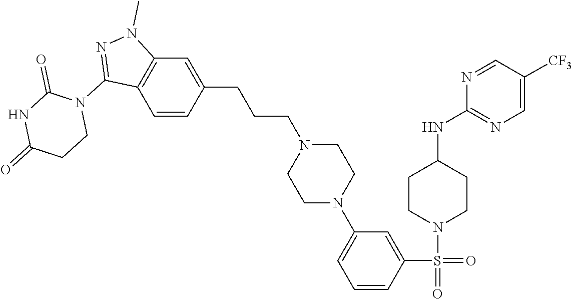 [SMILES: Cn1nc(N2CCC(=O)NC2=O)c2ccc(CCCN3CCN(c4cccc(S(=O)(=O)N5CCC(Nc6ncc(C(F)(F)F)cn6)CC5)c4)CC3)cc21] | 1-(1-methyl-6-(3-(4-(3-((4- ((5- (trifluoromethyl)pyrimidin- 2-yl)amino)piperidin-1- yl)sulfonyl)phenyl) piperazin-1-yl)propyl)- 1H-indazol- 3-yl)dihydropyrimidine- 2,4(1H,3H)-dione | 755.0 |
| 236 | 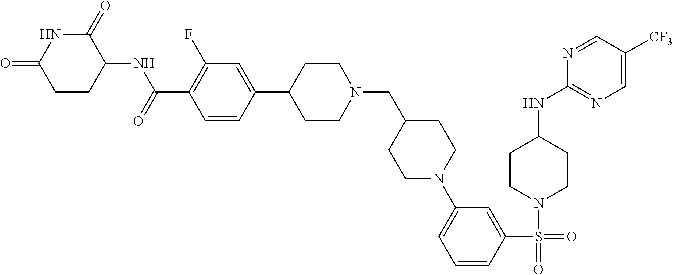 [SMILES: O=C1CCC(NC(=O)c2ccc(C3CCN(CC4CCN(c5cccc(S(=O)(=O)N6CCC(Nc7ncc(C(F)(F)F)cn7)CC6)c5)CC4)CC3)cc2F)C(=O)N1] | N-(2,6-dioxopiperidin-3-yl)- 2-fluoro-4-(1-((1-(3-((4-((5- (trifluoromethyl)pyrimidin- 2-yl)amino)piperidin-1- yl)sulfonyl)phenyl) piperidin-4-yl)methyl) piperidin-4- yl)benzamide | 815.0 |
| 237 | 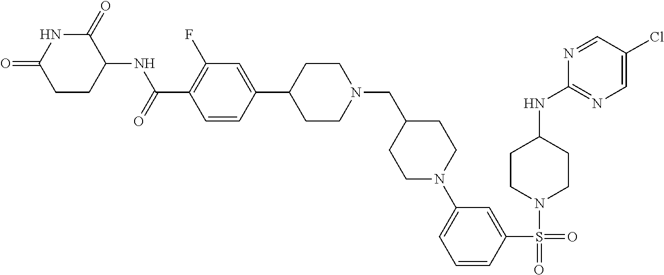 [SMILES: O=C1CCC(NC(=O)c2ccc(C3CCN(CC4CCN(c5cccc(S(=O)(=O)N6CCC(Nc7ncc(Cl)cn7)CC6)c5)CC4)CC3)cc2F)C(=O)N1] | 4-(1-((1-(3-((4-((5- chloropyrimidin-2- yl)amino)piperidin-1- yl)sulfonyl)phenyl) piperidin-4-yl)methyl) piperidin-4- yl)-N-(2,6-dioxopiperidin- 3-yl)-2-fluorobenzamide | 781.0 |
| 238 | 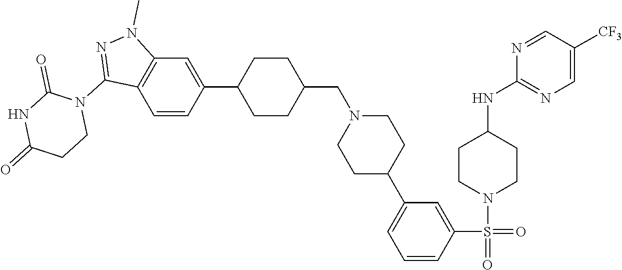 [SMILES: Cn1nc(N2CCC(=O)NC2=O)c2ccc(C3CCC(CN4CCC(c5cccc(S(=O)(=O)N6CCC(Nc7ncc(C(F)(F)F)cn7)CC6)c5)CC4)CC3)cc21] | 1-(1-methyl-6-(4-((4-(3-((4- ((5- (trifluoromethyl)pyrimidin- 2-yl)amino)piperidin-1- yl)sulfonyl)phenyl) piperidin-1-yl)methyl) cyclohexyl)- 1H-indazol-3- yl)dihydropyrimidine- 2,4(1H,3H)-dione | 808.1 |
| 239 | 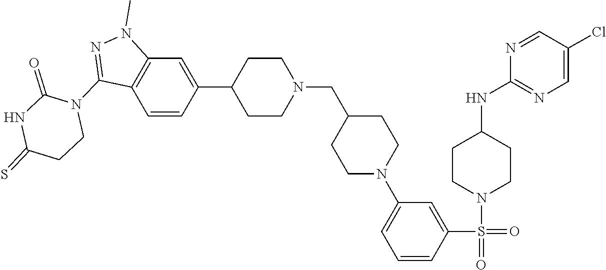 [SMILES: Cn1nc(N2CCC(=S)NC2=O)c2ccc(C3CCN(CC4CCN(c5cccc(S(=O)(=O)N6CCC(Nc7ncc(Cl)cn7)CC6)c5)CC4)CC3)cc21] | 1-(6-(1-((1-(3-((4-((5- chloropyrimidin-2- yl)amino)piperidin-1- yl)sulfonyl)phenyl) piperidin-4-yl)methyl) piperidin-4- yl)-1-methyl-1H-indazol- 3-yl)-4- thioxotetrahydropyrimidin- 2(1H)-one | 791.1 |
| 240 | 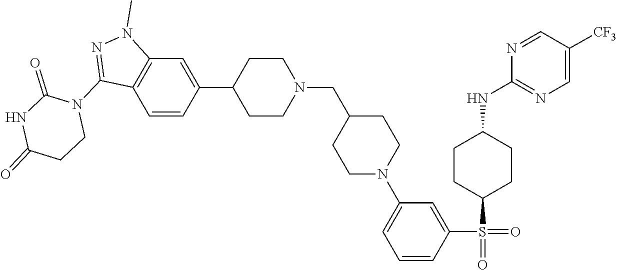 [SMILES: Cn1nc(N2CCC(=O)NC2=O)c2ccc(C3CCN(CC4CCN(c5cccc(S(=O)(=O)[C@H]6CC[C@H](Nc7ncc(C(F)(F)F)cn7)CC6)c5)CC4)CC3)cc21] | 1-(6-(1-((1-(3-(((1r,4r)-4- ((5-chloropyrimidin-2- yl)amino)cyclohexyl) sulfonyl)phenyl)piperidin- 4-yl)methyl)piperidin-4- yl)-1-methyl-1H-indazol- 3-yl)dihydropyrimidine- 2,4(1H,3H)-dione | 808.1 |
| 241 | 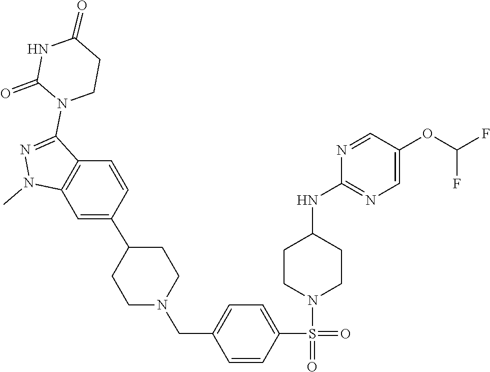 [SMILES: Cn1nc(N2CCC(=O)NC2=O)c2ccc(C3CCN(Cc4ccc(S(=O)(=O)N5CCC(Nc6ncc(OC(F)F)cn6)CC5)cc4)CC3)cc21] | 1-(6-(1-(4-((4-((5- (difluoromethoxy) pyrimidin-2-yl)amino) piperidin-1- yl)sulfonyl)benzyl) piperidin-4-yl)-1-methyl- 1H-indazol- 3-yl)dihydropyrimidine- 2,4(1H,3H)-dione | 724.0 |
| 242 | 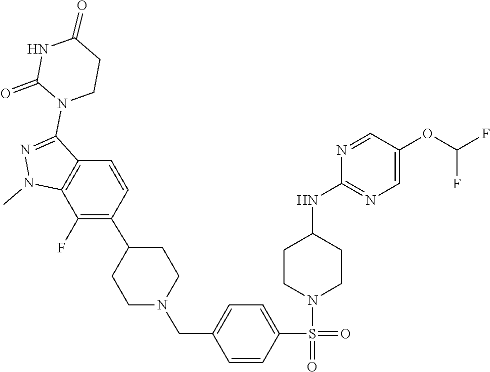 [SMILES: Cn1nc(N2CCC(=O)NC2=O)c2ccc(C3CCN(Cc4ccc(S(=O)(=O)N5CCC(Nc6ncc(OC(F)F)cn6)CC5)cc4)CC3)c(F)c21] | 1-(6-(1-(4-((4-((5- (difluoromethoxy) pyrimidin-2-yl)amino) piperidin-1- yl)sulfonyl)benzyl) piperidin-4-yl)-7- fluoro-1-methyl- 1H-indazol-3- yl)dihydropyrimidine- 2,4(1H,3H)-dione | 742.0 |
| 243 | 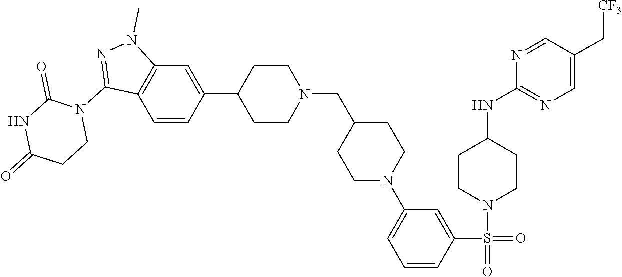 [SMILES: Cn1nc(N2CCC(=O)NC2=O)c2ccc(C3CCN(CC4CCN(c5cccc(S(=O)(=O)N6CCC(Nc7ncc(CC(F)(F)F)cn7)CC6)c5)CC4)CC3)cc21] | 1-(1-methyl-6-(1-((1-(3-((4- ((5-(2,2,2- trifluoroethyl)pyrimidin-2- yl)amino)piperidin-1- yl)sulfonyl)phenyl) piperidin-4-yl)methyl) piperidin-4- yl)-1H-indazol-3- yl)dihydropyrimidine- 2,4(1H,3H)-dione | 823.1 |
| 244 | 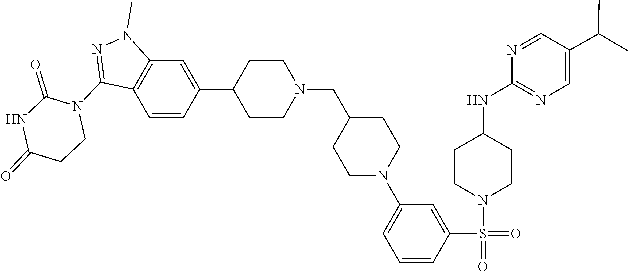 [SMILES: CC(C)c1cnc(NC2CCN(S(=O)(=O)c3cccc(N4CCC(CN5CCC(c6ccc7c(N8CCC(=O)NC8=O)nn(C)c7c6)CC5)CC4)c3)CC2)nc1] | 1-(6-(1-((1-(3-((4-((5- isopropylpyrimidin-2- yl)amino)piperidin-1- yl)sulfonyl)phenyl) piperidin-4-yl)methyl) piperidin-4- yl)-1-methyl-1H-indazol-3- yl)dihydropyrimidine- 2,4(1H,3H)-dione | 783.1 |
| 245 | 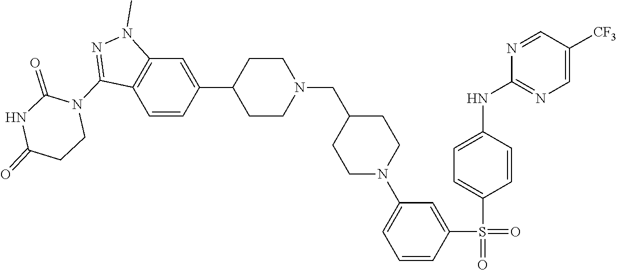 [SMILES: Cn1nc(N2CCC(=O)NC2=O)c2ccc(C3CCN(CC4CCN(c5cccc(S(=O)(=O)c6ccc(Nc7ncc(C(F)(F)F)cn7)cc6)c5)CC4)CC3)cc21] | 1-(1-methyl-6-(1- ((1-(3-((4-((5- (trifluoromethyl)pyrimidin- 2-yl)amino)phenyl)sulfonyl) phenyl)piperidin-4- yl)methyl)piperidin-4-yl)- 1H-indazol-3- yl)dihydropyrimidine- 2,4(1H,3H)-dione | 802.0 |
| 246 | 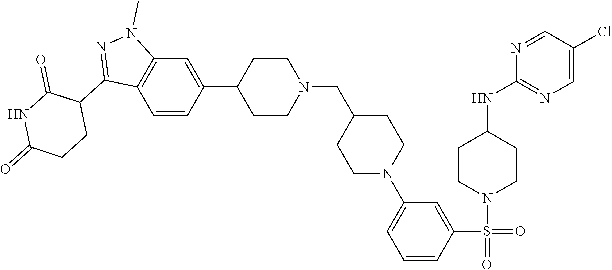 [SMILES: Cn1nc(C2CCC(=O)NC2=O)c2ccc(C3CCN(CC4CCN(c5cccc(S(=O)(=O)N6CCC(Nc7ncc(Cl)cn7)CC6)c5)CC4)CC3)cc21] | 3-(6-(1-((1-(3-((4-((5- chloropyrimidin-2- yl)amino)piperidin-1- yl)sulfonyl)phenyl)piperidin -4-yl)methyl)piperidin-4- yl)-1-methyl-1H-indazol-3- yl)piperidine-2,6-dione | 774.0 |
| 247 | 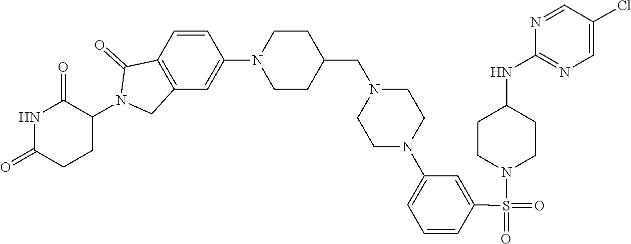 [SMILES: O=C1CCC(N2Cc3cc(N4CCC(CN5CCN(c6cccc(S(=O)(=O)N7CCC(Nc8ncc(Cl)cn8)CC7)c6)CC5)CC4)ccc3C2=O)C(=O)N1] | 3-(5-(4-((4-(3-((4-((5- chloropyrimidin-2- yl)amino)piperidin-1- yl)sulfonyl)phenyl) piperazin-1-yl)methyl) piperidin-1- yl)-1-oxoisoindolin-2- yl)piperidine-2,6-dione | 776.3 |
| 248 | 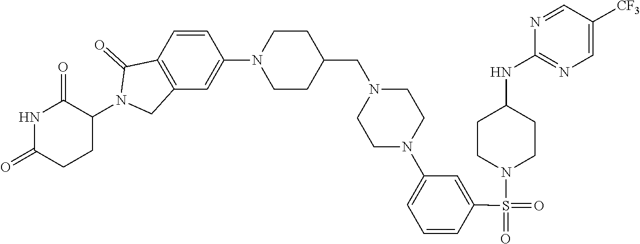 [SMILES: O=C1CCC(N2Cc3cc(N4CCC(CN5CCN(c6cccc(S(=O)(=O)N7CCC(Nc8ncc(C(F)(F)F)cn8)CC7)c6)CC5)CC4)ccc3C2=O)C(=O)N1] | 3-(1-oxo-5-(4-((4-(3-((4- ((5- (trifluoromethyl)pyrimidin- 2-yl)amino)piperidin-1- yl)sulfonyl)phenyl) piperazin-1-yl)methyl) piperidin-1- yl)isoindolin-2- yl)piperidine-2,6-dione | 810.0 |
| 249 | 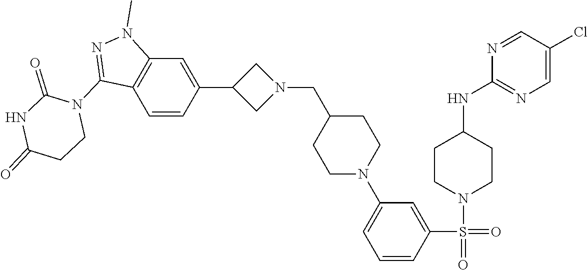 [SMILES: Cn1nc(N2CCC(=O)NC2=O)c2ccc(C3CN(CC4CCN(c5cccc(S(=O)(=O)N6CCC(Nc7ncc(Cl)cn7)CC6)c5)CC4)C3)cc21] | 1-(6-(1-((1-(3-((4-((5- chloropyrimidin-2- yl)amino)piperidin-1- yl)sulfonyl)phenyl) piperidin-4-yl)methyl) azetidin-3-yl)- 1-methyl-1H-indazol-3- yl)dihydropyrimidine- 2,4(1H,3H)-dione | 747.1 |
| 250 | 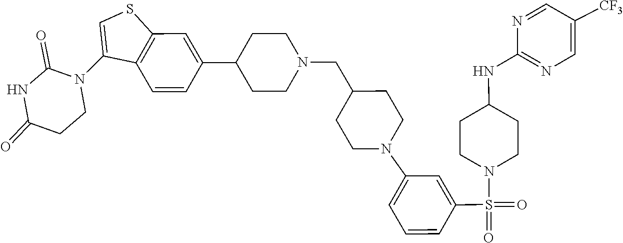 [SMILES: O=C1CCN(c2csc3cc(C4CCN(CC5CCN(c6cccc(S(=O)(=O)N7CCC(Nc8ncc(C(F)(F)F)cn8)CC7)c6)CC5)CC4)ccc23)C(=O)N1] | 3-(6-(1-((1-(3-((4-((5- (trifluoromethyl)pyrimidin- 2-yl)amino)piperidin-1- yl)sulfonyl)phenyl) piperidin-4-yl)methyl) piperidin-4- yl)benzo[b]thiophen-3- yl)piperidine-2,6-dione | 810.2 |
| 251 | 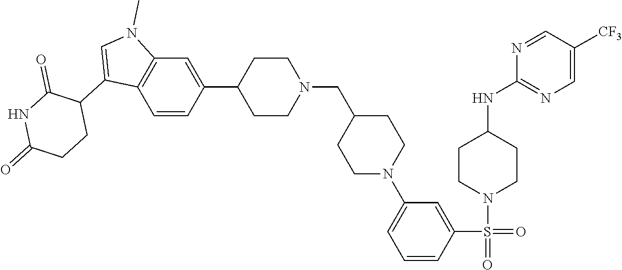 [SMILES: Cn1cc(C2CCC(=O)NC2=O)c2ccc(C3CCN(CC4CCN(c5cccc(S(=O)(=O)N6CCC(Nc7ncc(C(F)(F)F)cn7)CC6)c5)CC4)CC3)cc21] | 3-(1-methyl-6-(1- ((1-(3-((4-((5- (trifluoromethyl)pyrimidin- 2-yl)amino)piperidin-1- yl)sulfonyl)phenyl) piperidin-4-yl)methyl) piperidin-4- yl)-1H-indol-3- yl)piperidine-2,6-dione | 807.3 |
| 252 | 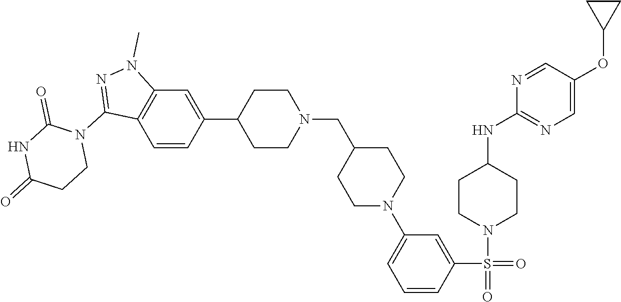 [SMILES: Cn1nc(N2CCC(=O)NC2=O)c2ccc(C3CCN(CC4CCN(c5cccc(S(=O)(=O)N6CCC(Nc7ncc(OC8CC8)cn7)CC6)c5)CC4)CC3)cc21] | 1-(6-(1-((1-(3-((4-((5- cyclopropoxypyrimidin-2- yl)amino)piperidin-1- yl)sulfonyl)phenyl) piperidin-4-yl)methyl) piperidin-4- yl)-1-methyl-1H-indazol-3- yl)dihydropyrimidine- 2,4(1H,3H)-dione | 797.3 |
| 253 | 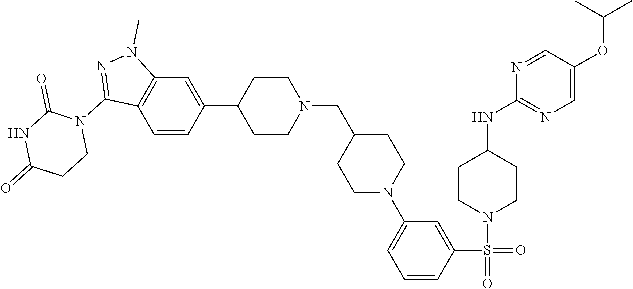 [SMILES: CC(C)Oc1cnc(NC2CCN(S(=O)(=O)c3cccc(N4CCC(CN5CCC(c6ccc7c(N8CCC(=O)NC8=O)nn(C)c7c6)CC5)CC4)c3)CC2)nc1] | 1-(6-(1-((1-(3-((4-((5- isopropoxypyrimidin-2- yl)amino)piperidin-1- yl)sulfonyl)phenyl) piperidin-4-yl)methyl) piperidin-4- yl)-1-methyl-1H-indazol-3- yl)dihydropyrimidine- 2,4(1H,3H)-dione | 799.3 |
| 254 | 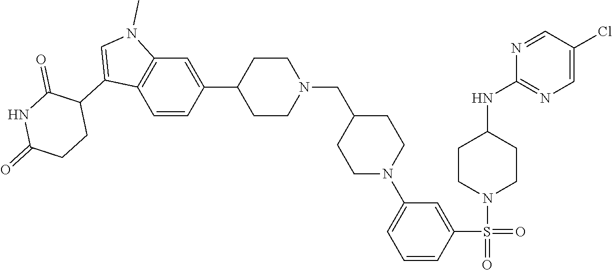 [SMILES: Cn1cc(C2CCC(=O)NC2=O)c2ccc(C3CCN(CC4CCN(c5cccc(S(=O)(=O)N6CCC(Nc7ncc(Cl)cn7)CC6)c5)CC4)CC3)cc21] | 3-(6-(1-((1-(3-((4-((5- chloropyrimidin-2- yl)amino)piperidin-1- yl)sulfonyl)phenyl) piperidin-4-yl)methyl) piperidin-4- yl)-1-methyl-1H-indol-3- yl)piperidine-2,6-dione | 773.3 |
| 255 | 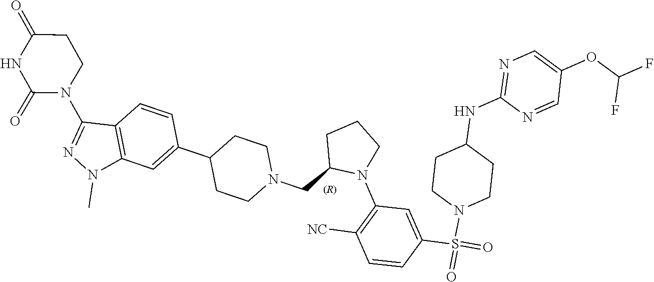 | (R)-4-((4-((5- (difluoromethoxy)pyrimidin- 2-yl)amino)piperidin-1- yl)sulfonyl)-2-(2-((4-(3- (2,4- dioxotetrahydropyrimidin- 1(2H)-yl)-1-methyl-1H- indazol-6-yl)piperidin-1- yl)methyl)pyrrolidin-1- yl)benzonitrile | 818.2 |
| 256 | 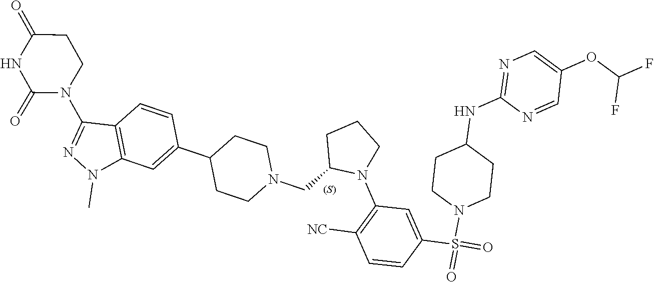 | (S)-4-((4-((5- (difluoromethoxy)pyrimidin- 2-yl)amino)piperidin-1- yl)sulfonyl)-2-(2-((4-(3- (2,4- dioxotetrahydropyrimidin- 1(2H)-yl)-1-methyl-1H- indazol-6-yl)piperidin-1- yl)methyl)pyrrolidin-1- yl)benzonitrile | 818.3 |
| 257 | 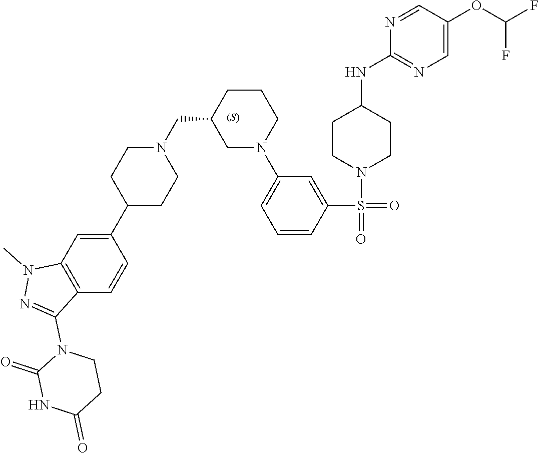 | (S)-1-(6-(1-((1-(3-((4-((5- (difluoromethoxy)pyrimidin- 2-yl)amino)piperidin-1- yl)sulfonyl)phenyl) piperidin-3-yl)methyl) piperidin-4- yl)-1-methyl-1H-indazol-3- yl)dihydropyrimidine- 2,4(1H,3H)-dione | 807.3 |
| 258 | 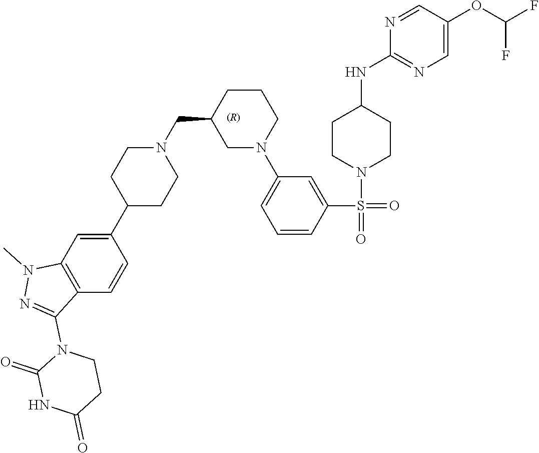 | (R)-1-(6-(1-((1-(3-((4-((5- (difluoromethoxy) pyrimidin-2-yl)amino) piperidin-1- yl)sulfonyl)phenyl) piperidin-3-yl)methyl) piperidin-4- yl)-1-methyl-1H-indazol-3- yl)dihydropyrimidine- 2,4(1H,3H)-dione | 807.3 |
| 259 | 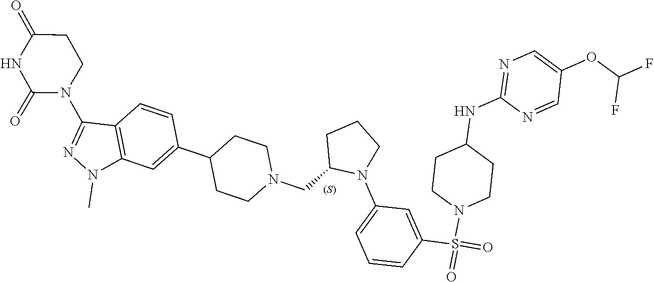 | (S)-1-(6-(1-((1-(3-((4-((5- (difluoromethoxy) pyrimidin-2-yl)amino) piperidin-1- yl)sulfonyl)phenyl) pyrrolidin-2-yl)methyl) piperidin-4- yl)-1-methyl-1H-indazol-3- yl)dihydropyrimidine- 2,4(1H,3H)-dione | 793.3 |
| 260 | 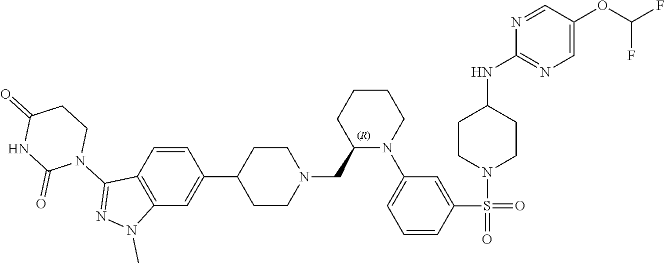 | (R)-1-(6-(1-((1-(3-((4-((5- (difluoromethoxy) pyrimidin-2-yl)amino) piperidin-1- yl)sulfonyl)phenyl) piperidin-2-yl)methyl) piperidin-4- yl)-1-methyl-1H-indazol-3- yl)dihydropyrimidine- 2,4(1H,3H)-dione | 807.3 |
| 261 | 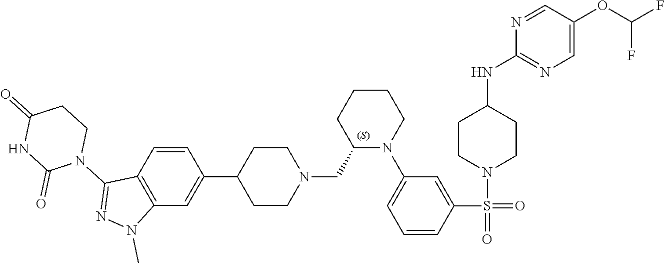 | (S)-1-(6-(1-((1-(3-((4-((5- (difluoromethoxy) pyrimidin-2-yl)amino) piperidin-1- yl)sulfonyl)phenyl) piperidin-2-yl)methyl) piperidin-4- yl)-1-methyl-1H-indazol-3- yl)dihydropyrimidine- 2,4(1H,3H)-dione | 807.3 |

Contemplated compounds of Formula (I)/(Ia) are shown in Compound Table 2 below:

TABLE 2

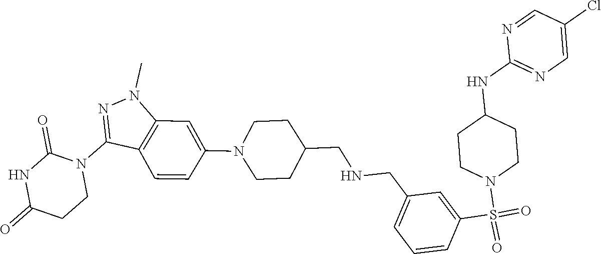 [SMILES: Cn1nc(N2CCC(=O)NC2=O)c2ccc(N3CCC(CNCc4cccc(S(=O)(=O)N5CCC(Nc6ncc(Cl)cn6)CC5)c4)CC3)cc21]

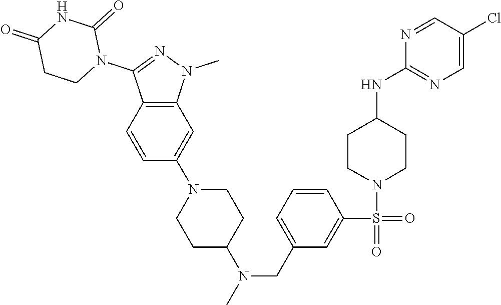 [SMILES: CN(Cc1cccc(S(=O)(=O)N2CCC(Nc3ncc(Cl)cn3)CC2)c1)C1CCN(c2ccc3c(N4CCC(=O)NC4=O)nn(C)c3c2)CC1]

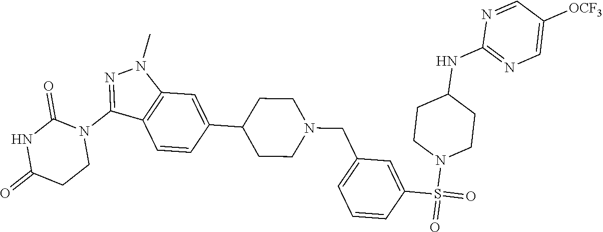 [SMILES: Cn1nc(N2CCC(=O)NC2=O)c2ccc(C3CCN(Cc4cccc(S(=O)(=O)N5CCC(Nc6ncc(OC(F)(F)F)cn6)CC5)c4)CC3)cc21]

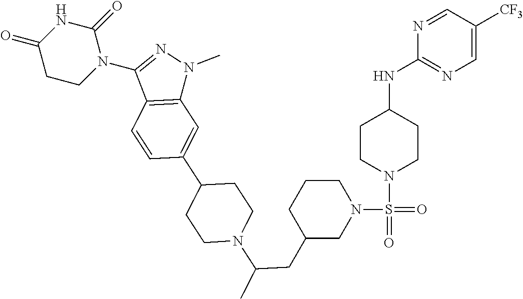 [SMILES: CC(CC1CCCN(S(=O)(=O)N2CCC(Nc3ncc(C(F)(F)F)cn3)CC2)C1)N1CCC(c2ccc3c(N4CCC(=O)NC4=O)nn(C)c3c2)CC1]

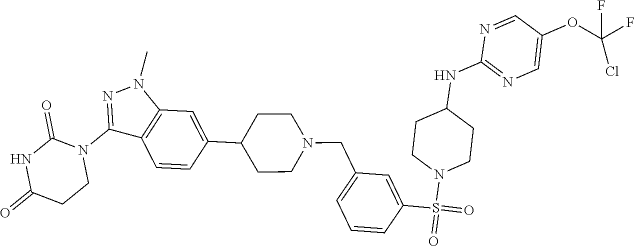 [SMILES: Cn1nc(N2CCC(=O)NC2=O)c2ccc(C3CCN(Cc4cccc(S(=O)(=O)N5CCC(Nc6ncc(OC(F)(F)Cl)cn6)CC5)c4)CC3)cc21]

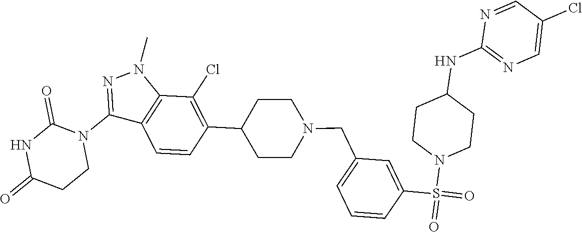 [SMILES: Cn1nc(N2CCC(=O)NC2=O)c2ccc(C3CCN(Cc4cccc(S(=O)(=O)N5CCC(Nc6ncc(Cl)cn6)CC5)c4)CC3)c(Cl)c21]

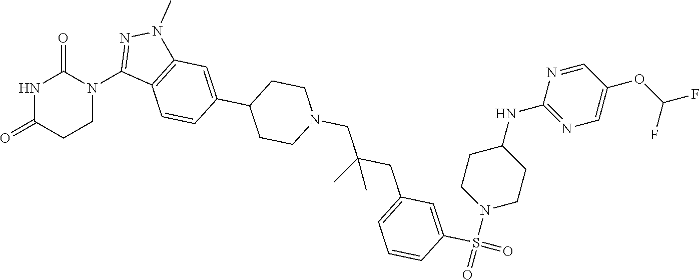 [SMILES: Cn1nc(N2CCC(=O)NC2=O)c2ccc(C3CCN(CC(C)(C)Cc4cccc(S(=O)(=O)N5CCC(Nc6ncc(OC(F)F)cn6)CC5)c4)CC3)cc21]

### General Synthetic Scheme

Compounds Formulas (A1), (A), (I), or (Ia) (and any embodiment thereof disclosed herein including specific compounds) can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds Formulas (A1), (A), (I), or (Ia) (and any embodiment thereof disclosed herein including specific compounds) can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C.

Compounds of Formula (Ia), where Degron is an E3 ligase ligand of formula (i)-(vi) where R^{2a} is hydrogen, ring Hy, R¹, R², and L are as defined in the fourth aspect, can be prepared as described in Scheme 1 below.

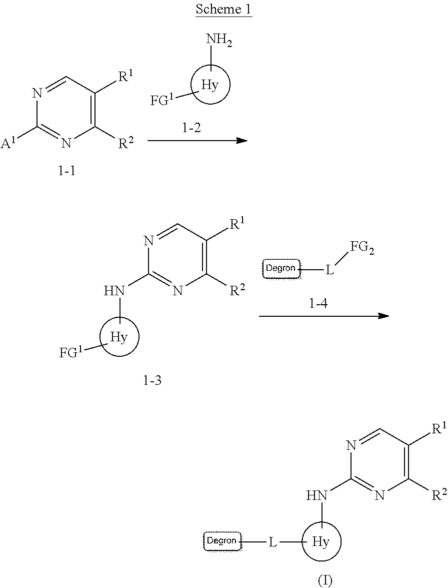

Treatment of a pyrimidine of formula 1-1 where A′ is a halogen such as chlorine, or bromine, with an amine of formula 1-2, where Hy is as defined in the Summary and FG¹ is a suitable functional group such as an acid or amine, under conditions well known in the art (such as in the presence of TEA and ZnCl₂ in tert-butanol), provides a compound of formula 1-3. A compound of formula 1-4 with a suitable functional group (FG²), such as acid, amine, or alkylhalide, reacts with FG¹ in 1-3 to afford compound of Formula (Ia). For example, the reaction is a peptide coupling reaction, where the resulting amide bond is part of L as defined in the Summary, and FG¹ and FG² are a combination of carboxylic acid and an amine, in the presence of suitable coupling reagents, such as a combination of HATU and DIPEA in DMF.

Alternatively, a compound of Formula (Ia) such as where R^{2a} is hydrogen, Hy is 1,4-piperidindiyl, Degron is a group of formula (i) and L is attached to Degron (i) via heterocyclylene and Hy via-SO₂—, can be synthesized as illustrated and described in Scheme 2.

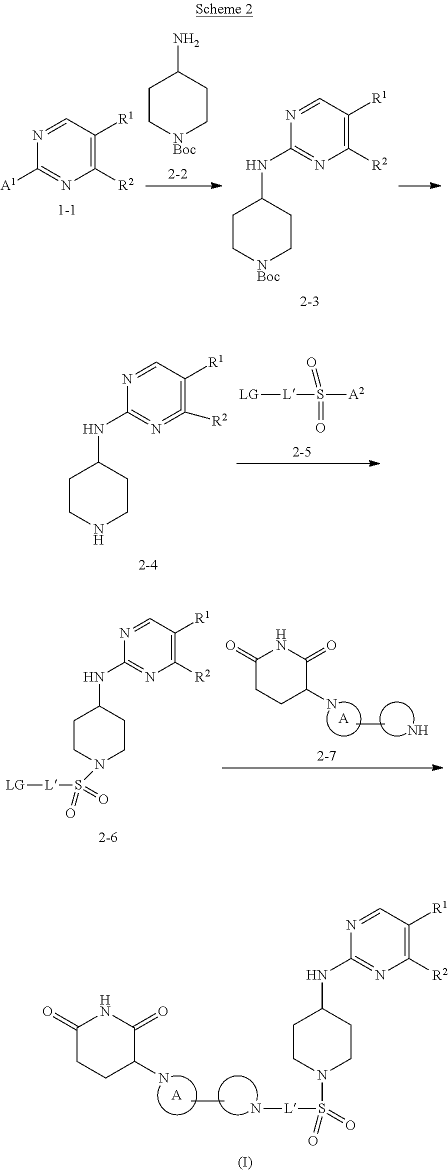

Treatment of a pyrimidine of formula 1-1 where A1 is a halogen such as chlorine, or bromine, with a piperidine amine of formula 2-2 under conditions well known in the art, such as in the presence of TEA, and ZnCl₂ in tert-butanol, provides a compound of formula 2-3. An amine compound of formula 2-4, prepared by removal of the Boc protecting group of 2-3 in the presence of an acid, such as TFA, is converted to a sulfonamide compound of formula 2-6 by treating it with a sulfonyl halide of formula 2-5 where L′ is a precursor group of L in the compound of Formula (Ia) as defined in Embodiment A3 and A2 is halogen such as chlorine and LG is a suitable leaving group such as halo or methylsulfonyl.

Treatment of a compound of formula 2-6 with an amine compound of formula 2-7, where ring A is defined as in Embodiment A3, under basic conditions such as in the presence of DIPEA, provides a compound of formula (I).

Compound of formula 1-1, 1-4, 2-5, and 2-7 are either commercially available or they can be prepared by methods known in the art.

Alternatively, a compound of Formula (Ia) such as where R^{2a} is hydrogen, Degron is a group of formula (i) and L is attached to Degron of formula (i) via heterocyclylene such as 4-piperidin-1-yl can be synthesized as illustrated and described in Scheme 3

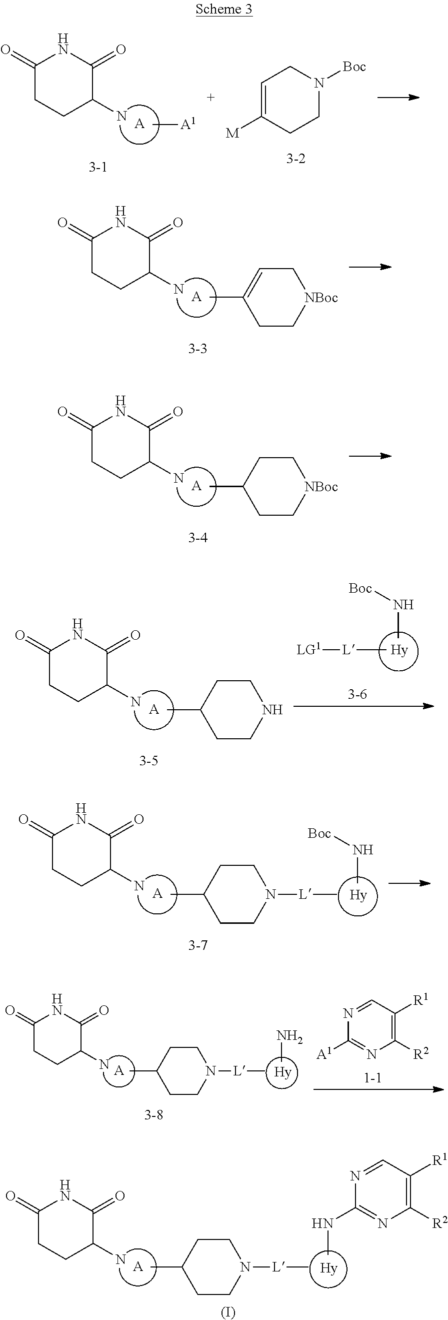

Cross coupling of a compound of formula 3-1, where A1 is a halogen and ring A as defined in the Summary, with a tetrahydropiperidinyl of formula 3-2 where M is a metal, such as boronic ester or zinc, provides a compound of formula 3-3. The reaction typically proceeds in the presence of a palladium catalyst; for example, when M is a boronic ester, a Suzuki reaction is conducted in the presence of Pd (dppf) C12 and Na₂CO₃, in 1,4-dioxane and water.

Reduction of the double bond in compound 3-3 under conditions well known in the art, such as in the presence of a palladium catalyst and under hydrogen atmosphere, provides compound of formula 3-4. Removal of the Boc protection group of 3-4 under acidic conditions provides an amine compound of formula 3-5. Reaction of 3-5 with a compound of formula 3-6, where LG¹ is a leaving group, such as halogen or —SO₂Me, Hy is as defined in the Summary and L′ is a precursor group of L as defined in the Summary, provides compound of formula 3-7. Removal of the Boc protecting group in compound 3-7 using an acid like TFA provides an amine compound of formula 3-8. Treatment of compound 3-8 with a compound of formula 1-1 under suitable conditions such as acidic, basic or transition metal catalyzed reaction conditions well known in the art, provides a compound of Formula (Ia).

Alternatively, compounds of Formula (I) or (Ia) where R^{2a} is hydrogen, R¹, R², Hy, L, and Degron are as defined in the Summary can be prepared as described in Scheme 4 below:

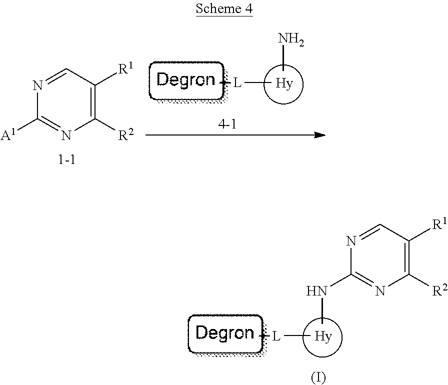

Treatment of a pyrimidine of formula 1-1 with an amine of formula 4-1, where R¹, R², Hy, L and Degron are as defined in the Summary, under suitable conditions such as acidic, basic or transition metal catalyzed reaction conditions well known in the art, provides a compound of Formula (I) or (Ia).

### Utility

The compound of Formulas (A1), (A), (I), or (Ia) (and any embodiment thereof disclosed herein including specific compounds) i.e., compound of this disclosure could cause degradation of CDK2 protein and hence are useful in the treatment of diseases mediated by CDK2.

Increasing evidence suggests that overactivated CDK2 leads to abnormal cell cycle regulation and proliferation in cancer cells. While CDK2 mutations are rarely found, the kinase activity of CDK2/Cyclin E or CDK2/Cyclin A complexes is elevated via several mechanisms in human cancers. Cyclin E has been found to be frequently amplified in human malignancies, for example, in uterine cancer, ovarian cancer, stomach cancer, and breast cancer. In some cancer types, loss-of-function mutations in FBXW7 or overexpression of USP28, which control the turnover of cyclin E, leads to cyclin E overexpression and CDK2 activation. Alternatively, certain cancer cells express a hyperactive, truncated form of cyclin E or cyclin A. In addition, cyclin A amplification and overexpression have also been reported in various cancers such as hepatocellular carcinomas, colorectal and breast cancers. In some tumors, catalytic activity of CDK2 is increased following loss of the expression or alteration of the location of the endogenous CDK2 inhibitor p27 or p21, or overexpression of SKP2, a negative regulator of p27. In addition, CDC25A and CDC25B, protein phosphatases responsible for the dephosphorylations that activate the CDK2, are overexpressed in various tumors. These various mechanisms of CDK2 activation have been validated using cancer cells or mouse cancer models.

Furthermore, CDK2/cyclin E phosphorylates oncogenic Myc to oppose ras-induced senescence, highlighting the importance of CDK2 in myc/ras-induced tumorigenesis. Inactivation of CDK2 has been shown to be synthetically lethal to myc over-expressing cancer cells. In aneuploid cancer cells, for example KRAS-mutant lung cancer, CDK2 inhibition resulted in anaphase catastrophe and apoptosis. Moreover, inhibiting CDK2 effectively induced granulocytic differentiation in AML cell lines and arrested tumor growth in AML mice models.

CDK2 activation as a result of cyclin E amplification or overexpression has also been identified as a key primary or acquired resistance pathway to tumors treated by CDK4/6 inhibitors or anti-HER2 therapy (i.e., trastuzumab). Accordingly, compounds of this disclosure can be used in combination with CDK4/6 inhibitors or anti-HER2 therapies for the treatment of cancers that become refractory to CDK4/6 inhibitors or anti-HER2 therapies.

Thus, compounds of this disclosure or a pharmaceutically acceptable salt thereof, may be useful for treating tumors characterized by one or more of: overexpression of CDK2, hyperphosphorylation of CDK2 (Thr160), amplification/overexpression of cyclin E or cyclin A, RB-deficiency, loss-of-function of mutation in FBXW7 or overexpression of USP28, expression of truncated cyclin E or cyclin A, dysregulation of p21 or p27 or overexpression of SKP2, amplification/overexpression of CDC25A or/and CDC25B, depletion of AMBRA1, hyperactive MYC/RAS, aneuploid cancers, CDK4 and/or CDK6 inhibitor refractory cancers. In some embodiments, the cancer is breast cancer (e.g. luminal A, triple negative, luminal B and Her2 positive), ovarian cancer (e.g. serous, clear cell, endometrioid, and mucinous ovarian carcinomas), uterine cancer (e.g. endometrial cancer and uterine sarcoma), stomach cancer (i.e. gastric cancer), lung cancer (e.g., adenocarcinoma, small cell lung cancer and non-small cell lung carcinomas, parvicellular and non-parvicellular carcinoma, bronchial carcinoma, bronchial adenoma, pleuropulmonary blastoma), renal cancer (e.g. clear cell renal cell carcinomas, papillary renal cell carcinomas, and chromophobe renal cell carcinomas), brain cancer (including astrocytoma, meningioma and glioblastoma), neuroblastoma, paraganglioma, pheochromocytoma, pancreatic neuroendocrine tumors, somatostatinomas, hemangioblastomas, gastrointestinal stromal tumors, pituitary tumors, leiomyomas, leiomyosarcomas, polycythaemia, retinal cancers, hereditary leiomyomatosis and renal cell cancer, astrocytoma, skin cancer (e.g. melanoma, squamous cell carcinoma, Kaposi sarcoma, Merkel cell skin cancer), bladder cancer (including bladder urothelial carcinoma), cervical cancer, colorectal cancer (e.g., cancer of the small intestine, colon cancer, rectal cancer, cancer of the anus), head and neck cancer (e.g., cancers of the larynx, hypopharynx, nasopharynx, oropharynx, lips, tongue and mouth), liver cancer (e.g., hepatocellular carcinoma, cholangiocellular carcinoma), prostate cancer, testicular cancer, gall bladder cancer, pancreatic cancer (e.g. exocrine pancreatic carcinoma, neuroendocrine pancreatic cancer), thyroid cancer, and parathyroid cancer, fallopian tube cancer, peritoneal cancer, vaginal cancer, biliary tract cancer, esophageal cancer (e.g. esophageal squamous cell carcinoma, esophageal adenocarcinoma), sarcoma (e.g. liposarcoma and osteosarcoma), bone cancer, chondrosarcoma, leukemia (including acute myeloid leukemia, acute lymphocytic leukemia, chronic myelogenous leukemia and chronic lymphocytic leukemia), lymphoma (e.g. non-Hodgkin lymphoma NHL including mantel cell lymphoma, MCL and Hodgkin lymphoma) and multiple myeloma. In some embodiments, the cancer is hepatocellular carcinomas, colorectal and breast cancers. In some embodiments, the cancer is ovarian cancer. In some such embodiments, the ovarian cancer is characterized by amplification or overexpression of CCNE1 and/or CCNE2.

In some embodiments, the cancer is breast cancer, including, e.g., ER-positive/HR-positive breast cancer, HER2-negative breast cancer; ER-positive/HR-positive breast cancer, HER2-positive breast cancer; ER-negative/HR-negative, HER2-positive breast cancer; triple negative breast cancer (TNBC); or inflammatory breast cancer. In some embodiments, the breast cancer is endocrine resistant breast cancer, anti-HER2 therapy (i.e., trastuzumab) resistant breast cancer, or breast cancer demonstrating primary or acquired resistance to CDK4/CDK6 inhibition. In some embodiments, the breast cancer, is advanced or metastatic breast cancer. In some embodiments of each of the foregoing, the breast cancer, is characterized by amplification or overexpression of CCNE1 CCNE2, and/or CCNA2. In some embodiments, the cancer is characterized by R^{b} deficiency and/or SKP2 amplification.

In some embodiments, the cancer can be lung cancer including NSCLC and SCLC. In some embodiments, the lung cancer can be characterized by R^{b} deficiency and/or SKP2 amplification.

In addition, compounds of this disclosure can also be useful in treating Ewing sarcoma, liposarcoma, osteosarcoma, rhabdomyosarcoma, neuroblastoma, medulloblastoma and AL in pediatric patients.

Besides cancer, CDK2 upregulation is also implicated in autoimmune diseases e.g., rheumatoid arthritis (RA), systemic lupus erythematosus (SLE), primary Sjogren's syndrome (pSS), multiple sclerosis (MS), Crohn's disease (CD), gout, uveitis, pemphigus vulgaris, and sepsis. As such compounds of this disclosure as described in the Summary as described in the first aspect (or any of the embodiments thereof herein above) are useful in treating above autoimmune diseases. Pharmacologic inhibition or genetic deletion of CDK2 has also been shown to preserve hearing function in animal models treated with cisplatin or noise (see Teitz T. et al., J Exp Med. 2018 Apr. 2; 215 (4): 1187-1203). Therefore, in addition to anti-tumor therapies, CDK2 inhibition can also be used as a promising preventive treatment for noise-, cisplatin-, or antibiotic-induced or age-related hearing loss.

### Testing

CDK2 potency and CDK2 degradation activities of the compounds of the present disclosure can be tested using the in vitro assays described in Biological Examples below.

### Pharmaceutical Compositions

In general, the compounds Formulas (A1), (A), (I), or (Ia) (unless stated otherwise, reference to compound/compounds of Formulas (A1), (A), (I), or (Ia) herein includes any embodiments thereof described herein or a pharmaceutically acceptable salt thereof) will be administered in a therapeutically effective amount by any of the accepted modes of administration for agents that serve similar utilities. Therapeutically effective amounts of compounds of Formulas (A1), (A), (I), or (Ia) (and any embodiment thereof disclosed herein including specific compounds) may range from about 0.01 to about 500 mg per kg patient body weight per day, which can be administered in single or multiple doses. A suitable dosage level may be from about 0.1 to about 250 mg/kg per day; about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount of the compound of Formulas (A1), (A), (I), or (Ia) (or any embodiment thereof disclosed herein including specific compounds), i.e., the active ingredient, will depend upon numerous factors such as the severity of the disease to be treated, the age and relative health of the patient, the potency of the compound being utilized, the route and form of administration, and other factors.

In general, compounds Formulas (A1), (A), (I), or (Ia) (and any embodiment thereof disclosed herein including specific compounds) will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance.

The compositions are comprised of in general, a compound of Formulas (A1), (A), (I), or (Ia) (or any embodiment thereof disclosed herein including specific compounds) in combination with at least one pharmaceutically acceptable excipient. Acceptable excipients are generally non-toxic, aid administration, and do not adversely affect the therapeutic benefit of the compound of Formulas (A1), (A), (I), or (Ia) (or any embodiment thereof disclosed herein including specific compounds). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

The compounds of Formulas (A1), (A), (I), or (Ia) (and any embodiment thereof disclosed herein including specific compounds) may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds of Formulas (A1), (A), (I), or (Ia) (and any embodiment thereof disclosed herein including specific compounds) may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds of Formulas (A1), (A), (1), or (Ia) (and any embodiment thereof disclosed herein including specific compounds) may also be formulated in rectal compositions such as suppositories or retention enemas, e.g., containing conventional suppository bases such as cocoa butter, polyethylene glycol, or other glycerides.

Certain compounds of Formulas (A1), (A), (I), or (Ia) (and any embodiment thereof disclosed herein including specific compounds) may be administered topically, that is by non-systemic administration. This includes the application of a compound of Formulas (A1), (A), (I), or (Ia) (or any embodiment thereof disclosed herein including specific compounds) externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds of Formulas (A1), (A), (I), or (Ia) (and any embodiment thereof disclosed herein including specific compounds) may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds of Formulas (A1), (A), (I), or (Ia) (and any embodiment thereof disclosed herein including specific compounds) may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator. Other suitable pharmaceutical excipients and their formulations are described in Remington's Pharmaceutical Sciences, edited by E. W. Martin (Mack Publishing Company, 20th ed., 2000).

The level of the compound of Formulas (A1), (A), (I), or (Ia) (or any embodiment thereof disclosed herein including specific compounds) in a formulation can vary within the full range employed by those skilled in the art. Typically, the formulation will contain, on a weight percent (wt. %) basis, from about 0.01-99.99 wt. % of a compound of Formulas (A1), (A), (I), or (Ia) (or any embodiment thereof disclosed herein including specific compounds) based on the total formulation, with the balance being one or more suitable pharmaceutical excipients. For example, the compound is present at a level of about 1-80 wt. %.

### Combinations and Combination Therapies

The compounds of Formulas (A1), (A), (I), or (Ia) (and any embodiment thereof disclosed herein including specific compounds) may be used in combination with one or more other drugs in the treatment of diseases or conditions for which compounds of Formulas (A1), (A), (I), or (Ia) (and any embodiment thereof disclosed herein including specific compounds) or the other drugs may have utility. Such other drug(s) may be administered, by a route and in an amount commonly used therefore, contemporaneously or sequentially with a compound of Formulas (A1), (A), (I), or (Ia) (or any embodiment thereof disclosed herein including specific compounds). When a compound of Formulas (A1), (A), (I), or (Ia) (or any embodiment thereof disclosed herein including specific compounds) is used contemporaneously with one or more other drugs, a pharmaceutical composition in unit dosage form containing such other drugs and the compound of Formulas (A1), (A), (I), or (Ia) (or any embodiment thereof disclosed herein including specific compounds) is preferred. However, the combination therapy may also include therapies in which the compound of Formulas (A1), (A), (I), or (Ia) (or any embodiment thereof disclosed herein including specific compounds) and one or more other drugs are administered on different overlapping schedules. It is also contemplated that when used in combination with one or more other active ingredients, the compounds of Formulas (A1), (A), (I), or (Ia) (and any embodiment thereof disclosed herein including specific compounds) and the other active ingredients may be used in lower doses than when each is used singly.

Accordingly, the pharmaceutical compositions of the present disclosure also include those that contain one or more other drugs, in addition to a compound of Formulas (A1), (A), (I), or (Ia) (or any embodiment thereof disclosed herein including specific compounds).

The above combinations include combinations of a compound of Formula (A1), (A), (I), or (Ia) (or any embodiment thereof disclosed herein including specific compounds) not only with one other drug, but also with two or more other active drugs. Likewise, a compound of Formula (A1), (A), (I), or (Ia) (or any embodiment thereof disclosed herein including specific compounds) may be used in combination with other drugs that are used in the prevention, treatment, control, amelioration, or reduction of risk of the diseases or conditions for which a compound of Formula (A1), (A), (I), or (Ia) (or any embodiment thereof disclosed herein including specific compounds) is useful. Such other drugs may be administered, by a route and in an amount commonly used therefore, contemporaneously or sequentially with a compound of Formula (A1), (A), (I), or (Ia) (or any embodiment thereof disclosed herein including specific compounds). When a compound of Formula (A1), (A), (I), or (Ia) (or any embodiment thereof disclosed herein including specific compounds) is used contemporaneously with one or more other drugs, a pharmaceutical composition containing such other drugs in addition to the compound of Formula (A1), (A), (I), or (Ia) (or any embodiment thereof disclosed herein including specific compounds) can be used. Accordingly, the pharmaceutical compositions of the present disclosure also include those that also contain one or more other active ingredients, in addition to a compound of Formula (A1), (A), (I), or (Ia) (or any embodiment thereof disclosed herein including specific compounds). The weight ratio of the compound of this disclosure to the second active ingredient may be varied and will depend upon the effective dose of each ingredient. Generally, an effective dose of each will be used.

Where the subject in need is suffering from or at risk of suffering from cancer, the subject can be treated with a compound of Formula (A1), (A), (I), or (Ia) (or any embodiment thereof disclosed herein including specific compounds) in any combination with one or more other anti-cancer agents including but not limited to: MAP kinase pathway (RAS/RAF/MEK/ERK) inhibitors including but not limited to: Vemurafanib (PLX4032), Dabrafenib, Encorafenib (LGX818), TQ-B3233, XL-518 (Cas No. 1029872-29-4, available from ACC Corp); trametinib, selumetinib (AZD6244), TQ-B3234, PD184352, PD325901, TAK-733, pimasertinib, binimetinib, refametinib, cobimetinib (GDC-0973), AZD8330, BVD-523, LTT462, Ulixertinib, AMG510, ARS853, and any RAS inhibitors disclosed in patents WO2016049565, WO2016164675, WO2016168540, WO2017015562, WO2017058728, WO2017058768, WO2017058792, WO2017058805, WO2017058807, WO2017058902, WO2017058915, WO2017070256, WO2017087528, WO2017100546, WO2017172979, WO2017201161, WO2018064510, WO2018068017, WO2018119183;
CSFIR inhibitors (PLX3397, LY3022855, etc.) and CSFIR antibodies (IMC-054, RG7155) TGF beta receptor kinase inhibitor such as LY2157299;
BTK inhibitor such as ibrutinib; BCR-ABL inhibitors: Imatinib (Gleevec®); Inilotinib hydrochloride; Nilotinib (Tasigna®); Dasatinib (BMS-345825); Bosutinib (SKI-606); Ponatinib (AP24534); Bafetinib (INNO406); Danusertib (PHA-739358), AT9283 (CAS 1133385-83-7); Saracatinib (AZD0530); and N-[2-[(1S,4R)-6-[[4-cyclobutylarmno)-5-(trifluoromethyl)-2-pyrimidinyl]amino]-l, 2,3,4-tetrahydronaphthalen-1,4-imin-9-yl]-2-oxoethyl]-acetamide (PF-03814735, CAS 942487-16-3);
ALK inhibitors: PF-2341066 (XALKOPJ®; crizotinib); 5-chloro-N4-(2-(isopropyl-sulfonyl)phenyl)-N2-(2-methoxy-4-(4-(4-methylpiperazin-l-yl)piperidin-l-yl)phenyl)pyrimidine-2,4-diamine; GSK1838705 A; CH5424802; Ceritinib (ZYKADIA); TQ-B3139, TQ-B3101 PI3K inhibitors: 4-[2-(lH-indazol-4-yl)-6-[[4-(methylsulfonyl)piperazin-l-yl]methyl]thieno[3,2-d]-pyrimidin-4-yl]morholine (also known as GDC 0941 and described in PCT Publication Nos. WO 09/036082 and WO 09/055730), 2-methyl-2-[4-[3-methyl-2-oxo-8-(quinolin-3-yl)-2,3-dihydro-imidazo[4,5-c]quinolin-l-yl]phenyl]propionitrile (also known as BEZ 235 or NVP-BEZ 235, and described in PCT Publication No. WO 06/122806);
Vascular Endothelial Growth Factor (VEGF) receptor inhibitors: Bevacizumab (sold under the trademark Avastin® by Genentech/Roche), axitinib, (N-methyl-2-[[3-[(E)-2-pyridin-2-ylethenyl]-lH-indazol-6-yl]sulfanyl]benzamide, also known as AG013736, and described in PCT Publication No. WO 01/002369), Brivanib Alaninate ((S)-((R)-l-(4-(4-fluoro-2-methyl-lH-indol-5-yloxy)-5-methylpyrrolo[2,l-f][l,2,4]triazin-6-yloxy)propan-2-yl) 2-aminopropanoate, also known as BMS-582664), motesanib (N-(2,3-dihydro-3,3-dimethyl-lH-indol-6-yl)-2-[(4-pyridinyl-methyl)amino]-3-pyridinecarboxamide, and described in PCT Publication No. WO 02/066470), pasireotide (also known as SOM230, and described in PCT Publication No. WO 02/010192), sorafenib (sold under the tradename Nexavar®); AL-2846 MET inhibitor such as foretinib, carbozantinib, or crizotinib;
FLT3 inhibitors-sunitinib malate (sold under the tradename Sutent® by Pfizer); PKC412 (midostaurin); tanutinib, sorafenib, lestaurtinib, KW-2449, quizartinib (AC220) and crenolanib;
Epidermal growth factor receptor (EGFR) inhibitors: Gefitnib (sold under the tradename Iressa®), N-[4-[(3-chloro-4-fluorophenyl)amino]-7-[[(3″S″)-tetrahydro-3-furanyl]oxy]-6-quinazolinyl]-4 (dimethylamino)-2-butenamide, sold under the tradename Tovok® by Boehringer Ingelheim), cetuximab (sold under the tradename Erbitux® by Bristol-Myers Squibb), panitumumab (sold under the tradename Vectibix® by Amgen);
HER2 receptor inhibitors: Trastuzumab (sold under the trademark Herceptin® by Genentech/Roche), Trastuzumab deruxtecan (sold under the trademark Enhertu), neratinib (also known as HKI-272, (2E)-N-[4-[[3-chloro-4-[(pyridin-2-yl) methoxy]phenyl]amino]-3-cyano-7-ethoxyquinolin-6-yl]-4-(dimethylamino)but-2-enamide, and described PCT Publication No. WO 05/028443), lapatinib or lapatinib ditosylate (sold under the trademark Tykerb® by GlaxoSmithKline); Trastuzumab emtansine (in the United States, ado-trastuzumab emtansine, trade name Kadcyla)—an antibody-drug conjugate consisting of the monoclonal antibody trastuzumab (Herceptin) linked to the cytotoxic agent mertansine (DM1);
HER dimerization inhibitors: Pertuzumab (sold under the trademark Omnitarg®, by Genentech);
CD20 antibodies: Rituximab (sold under the trademarks Riuxan®) and MabThera® by Genentech/Roche), tositumomab (sold under the trademarks Bexxar® by GlaxoSmithKline), ofatumumab (sold under the trademark Arzerra® by GlaxoSmithKline);
Tyrosine kinase inhibitors: Erlotinib hydrochloride (sold under the trademark Tarceva® by Genentech/Roche), Linifanib (N-[4-(3-amino-lH-indazol-4-yl)phenyl]-N′-(2-fluoro-5-methylphenyl) urea, also known as ABT 869, available from Genentech), sunitinib malate (sold under the tradename Sutent® by Pfizer), bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, and described in U.S. Pat. No. 6,780,996), dasatinib (sold under the tradename Sprycel® by Bristol-Myers Squibb), armala (also known as pazopanib, sold under the tradename Votrient® by GlaxoSmithKline), imatinib and imatinib mesylate (sold under the tradenames Gilvec® and Gleevec® by Novartis);
DNA Synthesis inhibitors: Capecitabine (sold under the trademark Xeloda® by Roche), gemcitabine hydrochloride (sold under the trademark Gemzar® by Eli Lilly and Company), nelarabine ((2R3S,4R,5R)-2-(2-amino-6-methoxy-purin-9-yl)-5-(hydroxymethyl)oxolane-3,4-diol, sold under the tradenames Arranon® and Atriance® by GlaxoSmithKline);
Antineoplastic agents: oxaliplatin (sold under the tradename Eloxatin® ay Sanofi-Aventis and described in U.S. Pat. No. 4,169,846);
Human Granulocyte colony-stimulating factor (G-CSF) modulators: Filgrastim (sold under the tradename Neupogen® by Amgen);
Immunomodulators: Afutuzumab (available from Roche®), pegfilgrastim (sold under the tradename Neulasta® by Amgen), lenalidomide (also known as CC-5013, sold under the tradename Revlimid®), thalidomide (sold under the tradename Thalomid®);
CD40 inhibitors: Dacetuzumab (also known as SGN-40 or huS2C6, available from Seattle Genetics, Inc); Pro-apoptotic receptor agonists (PARAs): Dulanermin (also known as AMG-951, available from Amgen/Genentech);
Hedgehog antagonists: 2-chloro-N-[4-chloro-3-(2-pyridinyl)phenyl]-4-(methylsulfonyl)-benzamide (also known as GDC-0449, and described in PCT Publication No. WO 06/028958);
Phospholipase A2 inhibitors: Anagrelide (sold under the tradename Agrylin®);
BCL-2 inhibitors: 4-[4-[[2-(4-chlorophenyl)-5,5-dimethyl-l-cyclohexen-l-yl]methyl]-l-piperazinyl]-N-[[4-[[(1R)-3-(4-morpholinyl)-l-[(phenylthio)methyl]propyl]amino]-3-[(trifluoromethyl)sulfonyl]phenyl]sulfonyl]benzamide (also known as ABT-263 and described in PCT Publication No. WO 09/155386);
MCI-1 inhibitors: MIK665, S64315, AMG 397, and AZD5991;
Aromatase inhibitors: Exemestane (sold under the trademark Aromasin® by Pfizer), letrozole (sold under the tradename Femara® by Novartis), anastrozole (sold under the tradename Arimidex®);
Topoisomerase I inhibitors: Irinotecan (sold under the trademark Camptosar® by Pfizer), topotecan hydrochloride (sold under the tradename Hycamtin® by GlaxoSmithKline);
Topoisomerase II inhibitors: etoposide (also known as VP-16 and Etoposide phosphate, sold under the tradenames Toposar®, VePesid® and Etopophos®), teniposide (also known as VM-26, sold under the tradename Vumon®);
mTOR inhibitors: Temsirolimus (sold under the tradename Torisel® by Pfizer), ridaforolimus (formally known as deferolimus, (1R,2R,4S)-4-[(2R)-2 [(1R,9S,12S,15R,16E, 18R,19R,21R,23S,24E,26E,28Z,30S,32S,35R)-1,18-dihydroxy-19,30-dimethoxy-15, 17, 21, 23, 29, 35-hexamethyl-2,3, 10, 14,20-pentaoxo-11, 36-dioxa-4-azatricyclo[30.3.1.0 4′9] hexatriaconta-16,24,26,28-tetraen-12-yl]propyl]-2-methoxycyclohexyl dimethylphosphinate, also known as AP23573 and MK8669, and described in PCT Publication No. WO 03/064383), everolimus (sold under the tradename Afinitor® by Novartis);
Proteasome inhibitor such as carfilzomib, MLN9708, delanzomib, or bortezomib;
BET inhibitors such as INCB054329, OTX015, and CPI-0610;
LSD1 inhibitors such as GSK2979552, and INCB059872;
HIF-2α inhibitors such as PT2977 and PT2385;
Osteoclastic bone resorption inhibitors: 1-Hydroxy-2-imidazol-1-yl-phosphonoethyl) phosphonic acid monohydrate (sold under the tradename Zometa® by Novartis); CD33 Antibody Drug Conjugates: Gemtuzumab ozogamicin (sold under the tradename Mylotarg® by Pfizer/Wyeth);
CD22 Antibody Drug Conjugates: Inotuzumab ozogamicin (also referred to as CMC-544 and WAY-207294, available from Hangzhou Sage Chemical Co., Ltd.);
CD20 Antibody Drug Conjugates: Ibritumomab tiuxetan (sold under the tradename Zevalin®);
Somatostain analogs: octreotide (also known as octreotide acetate, sold under the tradenames Sandostatin® and Sandostatin LAR®);
Synthetic Interleukin-11 (IL-11): oprelvekin (sold under the tradename Neumega® by Pfizer/Wyeth);
Synthetic erythropoietin: Darbepoetin alfa (sold under the tradename Aranesp® by Amgen);
Receptor Activator for Nuclear Factor κ B (RANK) inhibitors: Denosumab (sold under the tradename Prolia® by Amgen);
Thrombopoietin mimetic peptibodies: Romiplostim (sold under the tradename Nplate® by Amgen);
Cell growth stimulators: Palifermin (sold under the tradename Kepivance® by Amgen);
Anti-Insulin-like Growth Factor-1 receptor (IGF-1R) antibodies: Figitumumab (also known as CP-751,871, available from ACC Corp), robatumumab (CAS No. 934235-44-6);
Anti-CSI antibodies: Elotuzumab (HuLuc63, CAS No. 915296-00-3);
CD52 antibodies: Alemtuzumab (sold under the tradename Campath®);
Histone deacetylase inhibitors (HDI): Voninostat (sold under the tradename Zolinza® by Merck);
Alkylating agents: Temozolomide (sold under the tradenames Temodar® and Temodal® by Schering-Plough/Merck), dactinomycin (also known as actinomycin-D and sold under the tradename Cosmegen®), melphalan (also known as L-PAM, L-sarcolysin, and phenylalanine mustard, sold under the tradename Alkeran®), altretamine (also known as hexamethylmelamine (HMM), sold under the tradename Hexalen®), carmustine (sold under the tradename BiCNU®), bendamustine (sold under the tradename Treanda®), busulfan (sold under the tradenames Busulfex® and Myleran®), carboplatin (sold under the tradename Paraplatin®), lomustine (also known as CCNU, sold under the tradename CeeNU®), cisplatin (also known as CDDP, sold under the tradenames Platinol® and Platinol®-AQ), chlorambucil (sold under the tradename Leukeran®), cyclophosphamide (sold under the tradenames Cytoxan® and Neosar®), dacarbazine (also known as DTIC, DIC and imidazole carboxamide, sold under the tradename DTIC-Dome®), altretamine (also known as hexamethylmelamine (HMM) sold under the tradename Hexalen®), ifosfamide (sold under the tradename Ifex®), procarbazine (sold under the tradename Matulane®), mechlorethamine (also known as nitrogen mustard, mustine and mechloroethamine hydrochloride, sold under the tradename Mustargen®), streptozocin (sold under the tradename Zanosar®), thiotepa (also known as thiophosphoamide, TESPA and TSPA, sold under the tradename Thioplex®; Biologic response modifiers: bacillus calmette-guerin (sold under the tradenames theraCys® and TICE® BCG), denileukin diftitox (sold under the tradename Ontak®);
Anti-tumor antibiotics: doxorubicin (sold under the tradenames Adriamycin® and Rubex®), bleomycin (sold under the tradename Lenoxane®), daunorubicin (also known as dauorubicin hydrochloride, daunomycin, and rubidomycin hydrochloride, sold under the tradename Cerubidine®), daunorubicin liposomal (daunorubicin citrate liposome, sold under the tradename DaunoXome®), mitoxantrone (also known as DHAD, sold under the tradename Novantrone®), epirubicin (sold under the tradename Ellence™), idarubicin (sold under the tradenames Idamycin®, Idamycin PFS®), mitomycin C (sold under the tradename Mutamycin®);
Anti-microtubule agents: Estramustine (sold under the tradename Emcyl®);
Cathepsin K inhibitors: Odanacatib (also known as MK-0822, N-(1-cyanocyclopropyl)-4-fluoro-N-2-{(1S)-2,2,2-trifluoro-1-[4′-(methylsulfonyl) biphenyl-4-yl]ethyl}-L-leucinamide, available from Lanzhou Chon Chemicals, ACC Corp., and ChemieTek, and described in PCT Publication no. WO 03/075836); Epothilone B analogs: Ixabepilone (sold under the tradename LxempraR by Bristol-Myers Squibb);
Heat Shock Protein (HSP) inhibitors: Tanespimycin (17-allylamino-17-demethoxy-geldanamycin, also known as KOS-953 and 17-AAG, available from SIGMA, and described in U.S. Pat. No. 4,261,989), NVP-HSP990, AUY922, AT13387, STA-9090, Debio 0932, KW-2478, XL888, CNF2024, TAS-116
TpoR agonists: Eltrombopag (sold under the tradenames Promacta® and Revolade® by GlaxoSmithKline);
Anti-mitotic agents: Docetaxel (sold under the tradename Taxotere® by Sanofi-Aventis); Adrenal steroid inhibitors: aminoglutethimide (sold under the tradename Cytadren®);
Anti-androgens: Nilutamide (sold under the tradenames Nilandron® and Anandron®), bicalutamide (sold under tradename Casodex®), flutamide (sold under the tradename Fulexin™);
Androgens: Fluoxymesterone (sold under the tradename Halotestin®);
CDK (CDK1, CDK2, CDK3, CDK5, CDK7, CDK8, CDK9, CDK11/12, or CDK16) inhibitors including but not limited to Alvocidib (pan-CDK inhibitor, also known as flovopirdol or HMR-1275, 2-(2-chlorophenyl)-5,7-dihydroxy-8-[(3S,4R)-3-hydroxy-1-methyl-4-piperidinyl]-4-chromenone, and described in U.S. Pat. No. 5,621,002);
CDK4/6 inhibitors palbociclib, ribociclib, abemaciclib, and Trilaciclib; CDK9 inhibitors AZD 4573, P276-00, AT7519M, TP-1287; CDK2/4/6 inhibitor such as PF-06873600;
SHP-2 inhibitor such as TNO155;
MDM2/MDMX, MDM2/p53 and/or MDMX/p53 modulators;
Gonadotropin-releasing hormone (GnRH) receptor agonists: Leuprolide or leuprolide acetate (sold under the tradenames Viadure® by Bayer AG, Eligard® by Sanofi-Aventis and Lupron® by Abbott Lab);
Taxane anti-neoplastic agents: Cabazitaxel (1-hydroxy-7, 10-dimethoxy-9-oxo-5,20-epoxytax-l l-ene-2a,4,13a-triyl-4-acetate-2-benzoate-13-[(2R,3S)-3-{[(tert-butoxy)carbonyl]amino}-2-hydroxy-3-phenylpropanoate), larotaxel ((2α,3ξ,4α,5β,7α,10β,13α)-4,10-bis(acetyloxy)-13-({(2R,3S)-3-[(tert-butoxycarbonyl)amino]-2-hydroxy-3-phenylpropanoyl}oxy)-1-hydroxy-9-oxo-5,20-epoxy-7,19-cyclotax-l l-en-2-yl benzoate);
5HTla receptor agonists: Xaliproden (also known as SR57746, 1-[2-(2-naphthyl)ethyl]-4-[3-(trifluoromethyl)phenyl]-1,2,3,6-tetrahydropyridine, and described in U.S. Pat. No. 5,266,573); HPC vaccines: Cervarix® sold by GlaxoSmithKline, Gardasil® sold by Merck; Iron Chelating agents: Deferasinox (sold under the tradename Exjade® by Novartis);
Anti-metabolites: Claribine (2-chlorodeoxyadenosine, sold under the tradename Leustatin®), 5-fluorouracil (sold under the tradename Adrucil®), 6-thioguanine (sold under the tradename Purinethol®), pemetrexed (sold under the tradename Alimta®), cytarabine (also known as arabinosylcytosine (Ara-C), sold under the tradename Cytosar-U®), cytarabine liposomal (also known as Liposomal Ara-C, sold under the tradename DepoCyt™), decitabine (sold under the tradename Dacogen®), hydroxyurea (sold under the tradenames Hydrea®, Droxia™ and Mylocel™), fludarabine (sold under the tradename Fludara®), floxuridine (sold under the tradename FUDR®), cladribine (also known as 2-chlorodeoxyadenosine (2-CdA) sold under the tradename Leustatin™), methotrexate (also known as amethopterin, methotrexate sodium (MTX), sold under the tradenames Rheumatrex® and Trexall™), pentostatin (sold under the tradename Nipent®);
Bisphosphonates: Pamidronate (sold under the tradename Aredia®), zoledronic acid (sold under the tradename Zometa®); Demethylating agents: 5-azacitidine (sold under the tradename Vidaza®), decitabine (sold under the tradename Dacogen®);
Plant Alkaloids: Paclitaxel protein-bound (sold under the tradename Abraxane®), vinblastine (also known as vinblastine sulfate, vincaleukoblastine and VLB, sold under the tradenames Alkaban-AQ® and Velban®), vincristine (also known as vincristine sulfate, LCR, and VCR, sold under the tradenames Oncovin® and Vincasar Pfs®), vinorelbine (sold under the tradename Navelbine®), paclitaxel (sold under the tradenames Taxol and Onxal™);
Retinoids: Ali tretinoin (sold under the tradename Panretin®), tretinoin (all-trans retinoic acid, also known as ATRA, sold under the tradename Vesanoid®), Isotretinoin (13-cis-retinoic acid, sold under the tradenames AccutaneR, Amnesteem®, Claravis®, Clarus®, Decutan®, Isotane®, Izotech®, OrataneR, Isotret®, and Sotret®), bexarotene (sold under the tradename Targretin®);
Glucocorticosteroids: Hydrocortisone (also known as cortisone, hydrocortisone sodium succinate, hydrocortisone sodium phosphate, and sold under the tradenames Ala-Cort®, Hydrocortisone Phosphate, Solu-Cortef®, Hydrocort Acetate® and Lanacort®), dexamethazone ((8S,9R,10S,11S,13S,14S,16R,17R)-9-fluoro-l l,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-Methylprednisolone, Methylprednisolone Acetate, Methylprednisolone Sodium Succinate, sold under the tradenames Duralone®, Medralone®, Medrol®, M-Prednisol® and Solu-Medrol®);
Cytokines: interleukin-2 (also known as aldesleukin and IL-2, sold under the tradename Proleukin®), interleukin-11 (also known as oprevelkin, sold under the tradename Neumega®), alpha interferon alfa (also known as IFN-alpha, sold under the tradenames Intron® A, and Roferon-A®); [00209] Estrogen receptor downregulators: Fulvestrant (sold under the tradename Faslodex®);
Anti-estrogens: tamoxifen (sold under the tradename Novaldex®); Toremifene (sold under the tradename Fareston®);
Selective estrogen receptor modulators (SERMs): Raloxifene (sold under the tradename Evista®);
Leutinizing hormone releasing hormone (LHRH) agonists: Goserelin (sold under the tradename Zoladex®); Progesterones: megestrol (also known as megestrol acetate, sold under the tradename Megace®);
Miscellaneous cytotoxic agents: Arsenic trioxide (sold under the tradename Trisenox®), asparaginase (also known as L-asparaginase, Erwinia L-asparaginase, sold under the tradenames Elspar® and Kidrolase®);
One or more immune checkpoint inhibitors CD27, CD28, CD40, CD122, CD96, CD73, CD39, CD47, OX40, GITR, CSFIR, JAK, PI3K delta, PI3K gamma, TAM kinase, arginase, CD137 (also known as 4-1BB), ICOS, A2AR, A2BR, HIF-2α, B7-H3, B7-H4, BTLA, CTLA-4,
LAG3, TIM3, VISTA, CD96, TIGIT, PD-1, PD-L1 and PD-L2. In some embodiments, the immune checkpoint molecule is a stimulatory checkpoint molecule selected from CD27, CD28, CD40, ICOS, OX40, GITR, CD137 and STING. In some embodiments, the immune checkpoint molecule is an inhibitory checkpoint molecule selected from B7-H3, B7-H4, BTLA, CTLA-4, IDO, TDO, Arginase, KIR, LAG3, PD-1, TIM3, CD96, TIGIT and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP-224. In some embodiments, the anti-PD-1 monoclonal antibody is nivolumab, or pembrolizumab or PDR001. In some embodiments, the anti-PD1 antibody is pembrolizumab.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A (atezolizumab) or MEDI4736 (durvalumab).

In some embodiments, the inhibitor of an immune checkpoint molecule is an inhibitor of CTLA-4, e.g., an anti-CTLA-4 antibody. In some embodiments, the anti-CTLA-4 antibody is ipilimumab or tremelimumab. In some embodiments, the inhibitor of an immune checkpoint molecule is an inhibitor of LAG3, e.g., an anti-LAG3 antibody. In some embodiments, the anti-LAG3 antibody is BMS-986016 or LAG525. In some embodiments, the inhibitor of an immune checkpoint molecule is an inhibitor of GITR, e.g., an anti-GITR antibody. In some embodiments, the anti-GITR antibody is TRX518 or, MK-4166, INCAGN01876 or MK-1248. In some embodiments, the inhibitor of an immune checkpoint molecule is an inhibitor of OX40, e.g., an anti-OX40 antibody or OX40L fusion protein. In some embodiments, the anti-OX40 antibody is MEDI0562 or, INCAGN01949, GSK2831781, GSK-3174998, MOXR-0916, PF-04518600 or LAG525. In some embodiments, the OX40L fusion protein is MEDI6383

Compounds of Formulas (A1), (A), (I), or (Ia) (and any embodiment thereof disclosed herein including specific compounds) can also be used to increase or enhance an immune response, including increasing the immune response to an antigen; to improve immunization, including increasing vaccine efficacy; and to increase inflammation. In some embodiments, the compounds of the invention can be used to enhance the immune response to vaccines including, but not limited, Listeria vaccines, oncolytic viral vaccines, and cancer vaccines such as GVAX® (granulocyte-macrophage colony-stimulating factor (GM-CF) gene-transfected tumor cell vaccine). Anti-cancer vaccines include dendritic cells, synthetic peptides, DNA vaccines and recombinant viruses. Other immune-modulatory agents also include those that block immune cell migration such as antagonists to chemokine receptors, including CCR2 and CCR4; Sting agonists and Toll receptor agonists.

Other anti-cancer agents also include those that augment the immune system such as adjuvants or adoptive T cell transfer. Compounds of this application may be effective in combination with CAR (Chimeric antigen receptor) T cell treatment as a booster for T cell activation.

A compound of Formula (A1), (A), (I), or (Ia) (or any embodiment thereof disclosed herein including specific compounds) can also be used in combination with the following adjunct therapies: anti-nausea drugs: NK-1 receptor antagonists: Casopitant (sold under the tradenames Rezonic® and Zunrisa® by GlaxoSmithKline); and
Cytoprotective agents: Amifostine (sold under the tradename Ethyol®), leucovorin (also known as calcium leucovorin, citrovorum factor and folinic acid).

### EXAMPLES

The following preparations of Intermediates (References) and compounds of Formula (Ia) (Examples) are given to enable those skilled in the art to more clearly understand and to practice the present disclosure. They should not be considered as limiting the scope of the disclosure, but merely as being illustrative and representative thereof.

### Reference 1

### Synthesis of 4-((14-amino-3,6,9,12-tetraoxatetradecyl)amino)-2-(2,6-dioxopiperidin-3-yl)isoindoline-1,3-dione, 2,2,2-trifluoroacetate

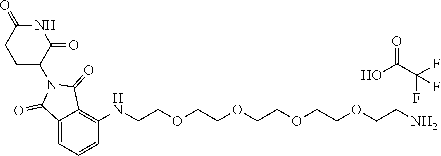

### Step 1: tert-Butyl (14-((2-(2,6-dioxopiperidin-3-yl)-1,3-dioxoisoindolin-4-yl)amino)-3,6,9,12-tetraoxatetradecyl)carbamate

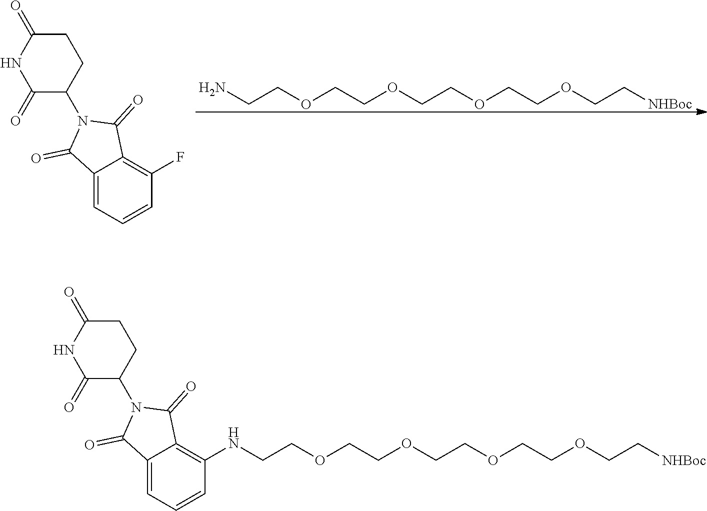 [SMILES: O=C1CCC(N2C(=O)c3cccc(F)c3C2=O)C(=O)N1 ; CC(C)(C)OC(=O)NCCOCCOCCOCCOCCN ; CC(C)(C)OC(=O)NCCOCCOCCOCCOCCNc1cccc2c1C(=O)N(C1CCC(=O)NC1=O)C2=O]

A mixture of 2-(2,6-dioxopiperidin-3-yl)-4-fluoroisoindoline-1,3-dione (500.0 mg, 1.81 mmol, 1.00 eq.), tert-butyl (14-amino-3,6,9,12-tetraoxatetradecyl)carbamate (609.0 mg, 1.81 mmol, 1.00 eq.), DIPEA (467.9 mg, 3.62 mmol, 2.00 eq.) in DMF (6.0 mL) was stirred for 16 h at 90° C. under nitrogen atmosphere. The mixture was cooled, diluted with water, and then extracted with EtOAc. The combined organic layer was washed with water, dried over anhydrous Na₂SO₄, filtered, and then concentrated. The residue was purified by silica gel column chromatography, eluted with PE/EtOAc (5:1), to afford the title compound as a yellow solid.

### Step 2: 4-((14-Amino-3,6,9,12-tetraoxatetradecyl)amino)-2-(2,6-dioxopiperidin-3-yl)isoindoline-1,3-dione, 2,2,2-trifluoroacetate

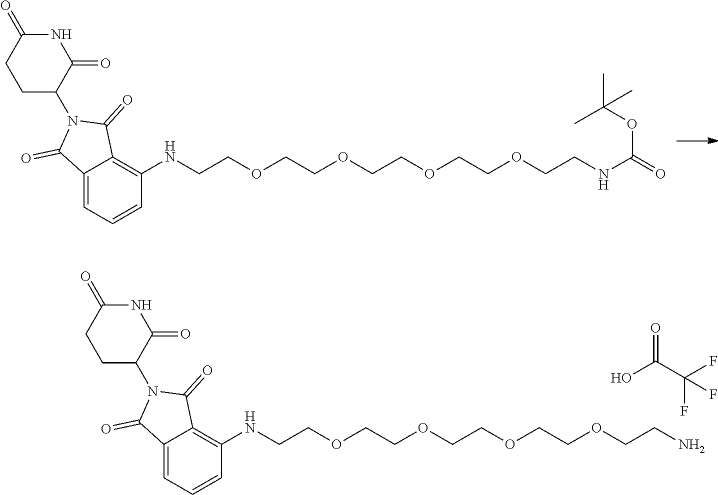

TFA (0.3 mL, 3.92 mmol, 46.67 eq.) was added to a stirred solution of tert-butyl (14-((2-(2,6-dioxopiperidin-3-yl)-1,3-dioxoisoindolin-4-yl)amino)-3,6,9,12-tetraoxatetradecyl)carbamate (50 mg, 0.084 mmol, 1.00 eq.) in DCM (1.0 mL) at 0° C. under nitrogen atmosphere. The mixture was stirred for 2 h, and then concentrated to give crude title compound as light yellow oil.

### Reference 2

### Synthesis of 2-(2-(2-(2-((2-(2,6-dioxopiperidin-3-yl)-1,3-dioxoisoindolin-4-yl)oxy)-acetamido)ethoxy) ethoxy)ethyl methanesulfonate

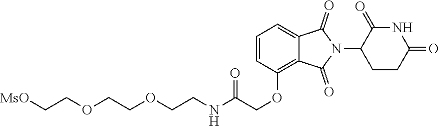 [SMILES: CS(=O)(=O)OCCOCCOCCNC(=O)COc1cccc2c1C(=O)N(C1CCC(=O)NC1=O)C2=O]

### Step 1: tert-Butyl 2-((2-(2,6-dioxopiperidin-3-yl)-1,3-dioxoisoindolin-4-yl)oxy)acetate

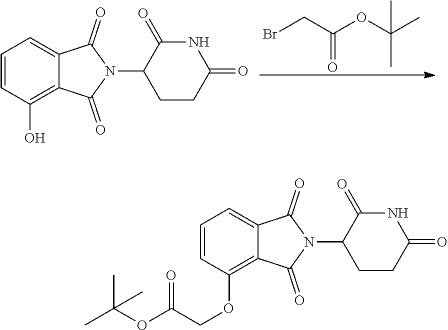

A mixture of 2-(2,6-dioxopiperidin-3-yl)-4-hydroxyisoindoline-1,3-dione (1.5 g, 5.47 mmol, 1.00 eq.), tert-butyl 2-bromoacetate (1.3 g, 6.66 mmol, 1.22 eq.) and K₂CO₃ (1.1 g, 7.96 mmol, 1.46 eq.) in DMF (20.0 mL) was stirred at RT for 2 h. The mixture was diluted with H₂O and extracted with ethyl acetate. The combined organic phase was washed with brine, dried over Na₂SO₄, concentrated to get title compound as a white solid.

### Step 2: 2-((2-(2,6-Dioxopiperidin-3-yl)-1,3-dioxoisoindolin-4-yl)oxy)acetic acid

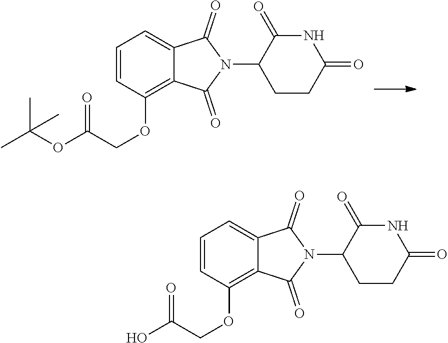 [SMILES: CC(C)(C)OC(=O)COc1cccc2c1C(=O)N(C1CCC(=O)NC1=O)C2=O ; O=C(O)COc1cccc2c1C(=O)N(C1CCC(=O)NC1=O)C2=O]

A solution of tert-butyl 2-((2-(2,6-dioxopiperidin-3-yl)-1,3-dioxoisoindolin-4-yl)oxy)-acetate (1.0 g, 2.57 mmol, 1.00 eq.) and TFA (5.0 mL) in DCM (10.0 mL) was stirred at rt for 2 h. The mixture was concentrated and the residue was triturated with ether to get title compound as a white solid.

### Step 3: 2-((2-(2,6-Dioxopiperidin-3-yl)-1,3-dioxoisoindolin-4-yl)oxy)-N-(2-(2-(2-hydroxyethoxy) ethoxy)ethyl)acetamide

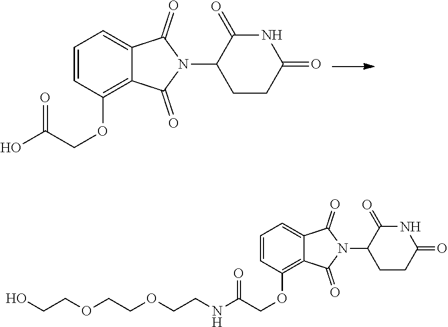 [SMILES: O=C(O)COc1cccc2c1C(=O)N(C1CCC(=O)NC1=O)C2=O ; O=C(COc1cccc2c1C(=O)N(C1CCC(=O)NC1=O)C2=O)NCCOCCOCCO]

HATU (513 mg, 1.35 mmol, 1.5 eq) was added to a stirred solution of 2-((2-(2,6-dioxopiperidin-3-yl)-1,3-dioxoisoindolin-4-yl)oxy)acetic acid (300 mg, 0.90 mmol, 1.00 eq.), 2-(2-(2-aminoethoxy) ethoxy) ethanol (201 mg, 1.35 mmol, 1.50 eq.) and DIPEA (348 mg, 2.69 mmol, 2.99 eq.) in DMF (6.0 mL) at 0° C. and the mixture was stirred at RT for 1 h. The mixture was diluted with H₂O and extracted with DCM. The combined organic phase was washed with brine, dried over Na₂SO₄, concentrated to get crude title compound as a yellow oil, which was used for next step without further purification.

### Step 4: 2-(2-(2-(2-((2-(2,6-Dioxopiperidin-3-yl)-1,3-dioxoisoindolin-4-yl)oxy)acetamido)ethoxy) ethoxy)ethyl methanesulfonate

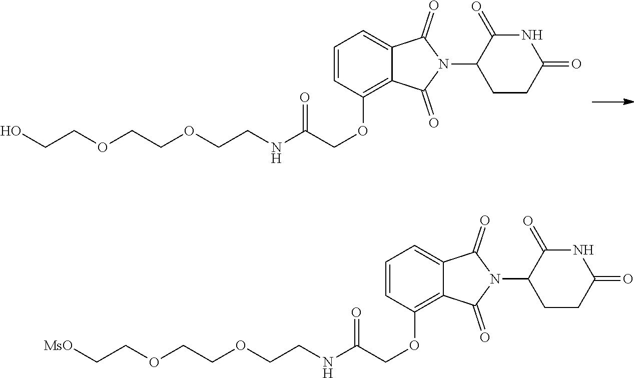 [SMILES: O=C(COc1cccc2c1C(=O)N(C1CCC(=O)NC1=O)C2=O)NCCOCCOCCO ; CS(=O)(=O)OCCOCCOCCNC(=O)COc1cccc2c1C(=O)N(C1CCC(=O)NC1=O)C2=O]

MsCl (298 mg, 2.60 mmol, 1.50 eq.) was added to a stirred solution of 2-((2-(2,6-dioxopiperidin-3-yl)-1,3-dioxoisoindolin-4-yl)oxy)-N-(2-(2-(2-hydroxyethoxy)ethoxy)ethyl)acetamide (800 mg, 1.73 mmol, 1.00 eq.) and TEA (524 mg, 5.18 mmol, 2.99 eq.) in DCM (8.0 mL) at 0° C. and the mixture was stirred at 0° C. for 1 h. The mixture was diluted with H₂O and extracted with DCM. The combined organic phase was washed with brine, dried over Na₂SO₄, concentrated and purified by flash silica gel chromatography (DCM:MeOH=50:1) to get title compound as a white solid.

### Reference 3

### Synthesis of 2-(2-(2-((2-(2,6-dioxopiperidin-3-yl)-1,3-dioxoisoindolin-4-yl)oxy)acetamido)ethoxy)-ethyl methanesulfonate

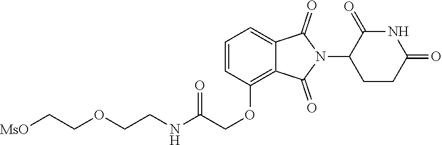 [SMILES: CS(=O)(=O)OCCOCCNC(=O)COc1cccc2c1C(=O)N(C1CCC(=O)NC1=O)C2=O]

### Step 1: 2-((2-(2,6-Dioxopiperidin-3-yl)-1,3-dioxoisoindolin-4-yl)oxy)-N-(2-(2-hydroxyethoxy)-ethyl)acetamide

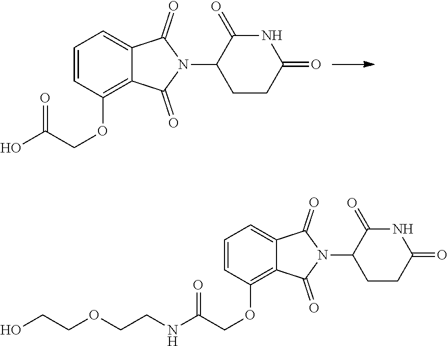 [SMILES: O=C(O)COc1cccc2c1C(=O)N(C1CCC(=O)NC1=O)C2=O ; O=C(COc1cccc2c1C(=O)N(C1CCC(=O)NC1=O)C2=O)NCCOCCO]

A mixture of 2-((2-(2,6-dioxopiperidin-3-yl)-1,3-dioxoisoindolin-4-yl)oxy)acetic acid (180 mg, 0.54 mmol, 1.00 eq.), 2-(2-aminoethoxy)ethan-1-ol (85 mg, 0.81 mmol, 1.50 eq.), HATU (308 mg, 0.81 mmol, 1.50 eq.) and DIPEA (209 mg, 1.62 mmol, 3.00 eq.) in DMF (5.0 mL) was stirred at 0° C. for 1 h. The reaction mixture was diluted with H₂O and extracted with DCM. The combined organic phase was washed with brine, dried over Na₂SO₄ and concentrated to give crude title compound as a yellow oil, which was used for next step without further purification.

### Step 2: 2-(2-(2-((2-(2,6-Dioxopiperidin-3-yl)-1,3-dioxoisoindolin-4-yl)oxy)acetamido)ethoxy)-ethyl methanesulfonate

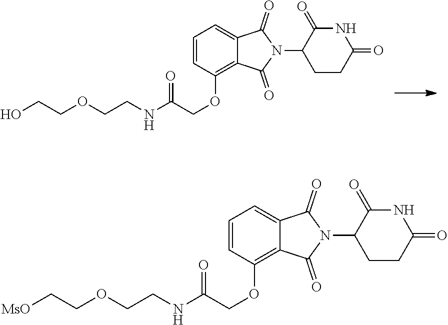 [SMILES: O=C(COc1cccc2c1C(=O)N(C1CCC(=O)NC1=O)C2=O)NCCOCCO ; CS(=O)(=O)OCCOCCNC(=O)COc1cccc2c1C(=O)N(C1CCC(=O)NC1=O)C2=O]

MsCl (162 mg, 1.41 mmol, 1.48 eq.) was added slowly to a stirred solution of 2-((2-(2,6-dioxopiperidin-3-yl)-1,3-dioxoisoindolin-4-yl)oxy)-N-(2-(2-hydroxyethoxy)ethyl)acetamide (400 mg, 0.95 mmol, 1.00 eq.) and TEA (288 mg, 2.85 mmol, 3.00 eq.) in DCM (8.0 mL) at 0° C. The resulting mixture was stirred at 0° C. for 1 h, diluted with H₂O and extracted with DCM. The combined organic phase was washed with brine, dried over Na₂SO₄, concentrated and the residue was purified by flash silica gel chromatography (DCM:MeOH=50:1) to give the title compound as a white solid.

### Reference 4

### Synthesis of 1-((2-(2,6-dioxopiperidin-3-yl)-1,3-dioxoisoindolin-4-yl)oxy)-2-oxo-6,9,12-trioxa-3-azatetradecan-14-yl methanesulfonate

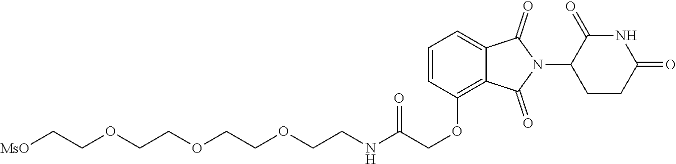 [SMILES: CS(=O)(=O)OCCOCCOCCOCCNC(=O)COc1cccc2c1C(=O)N(C1CCC(=O)NC1=O)C2=O]

### Step 1: 2-((2-(2,6-Dioxopiperidin-3-yl)-1,3-dioxoisoindolin-4-yl)oxy)-N-(2-(2-(2-(2-hydroxyethoxy)ethoxy)ethoxy)ethyl)acetamide

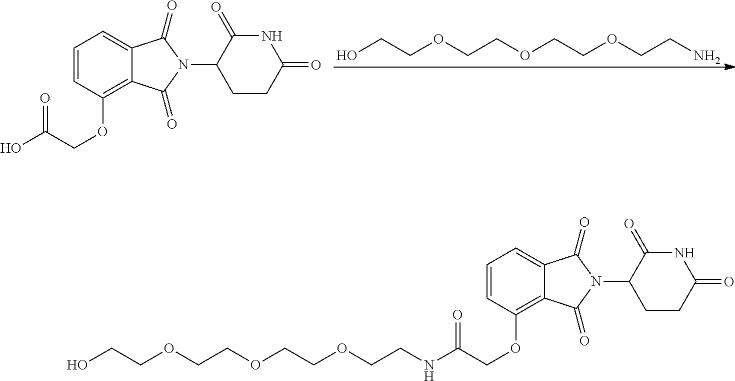

HATU (513 mg, 1.35 mmol, 1.50 eq.) and DIPEA (348 mg, 2.69 mmol, 3.00 eq.) were added to a mixture of 2-((2-(2,6-dioxopiperidin-3-yl)-1,3-dioxoisoindolin-4-yl)oxy)acetic acid (300 mg, 0.90 mmol, 1.00 eq.) and 2-(2-(2-(2-aminoethoxy) ethoxy) ethoxy) ethan-1-ol (259 mg, 1.34 mmol, 1.49 eq.) in DMF (5.0 mL) at 0° C. The mixture was stirred at 0° C. for 1 h, diluted with H₂O, and then extracted with DCM. The combined organic phase was washed with brine, dried over Na₂SO₄, filtered and concentrated to get crude title compound as a yellow oil, which was used for next step without further purification.

### Step 2: 1-((2-(2,6-Dioxopiperidin-3-yl)-1,3-dioxoisoindolin-4-yl)oxy)-2-oxo-6,9,12-trioxa-3-azatetradecan-14-yl methanesulfonate

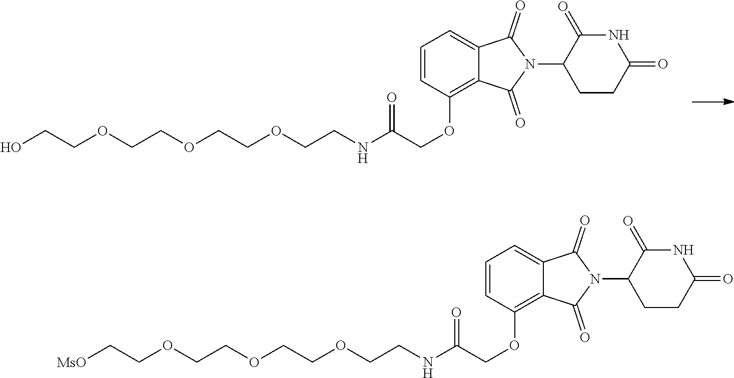 [SMILES: O=C(COc1cccc2c1C(=O)N(C1CCC(=O)NC1=O)C2=O)NCCOCCOCCOCCO ; CS(=O)(=O)OCCOCCOCCOCCNC(=O)COc1cccc2c1C(=O)N(C1CCC(=O)NC1=O)C2=O]

MsCl (271 mg, 2.37 mmol, 1.50 eq.) was added slowly to a stirred solution of 2-((2-(2,6-dioxopiperidin-3-yl)-1,3-dioxoisoindolin-4-yl)oxy)-N-(2-(2-(2-(2-hydroxyethoxy) ethoxy)-ethoxy)ethyl)acetamide (800 mg, 1.58 mmol, 1.00 eq.) and TEA (479 mg, 4.73 mmol, 3.00 eq.) in DCM (8.0 mL) at 0° C. The mixture was stirred at 0° C. for 1 h, diluted with H₂O and extracted with DCM. The combined organic phase was washed with brine, dried over Na₂SO₄, concentrated and purified by flash silica gel chromatography (DCM:MeOH=50:1) to get the title compound as a white solid.

### Reference 6

### Synthesis of 1-((2-(2,6-dioxopiperidin-3-yl)-1,3-dioxoisoindolin-4-yl)oxy)-2-oxo-6,9,12,15-tetraoxa-3-azaheptadecan-17-yl methanesulfonate

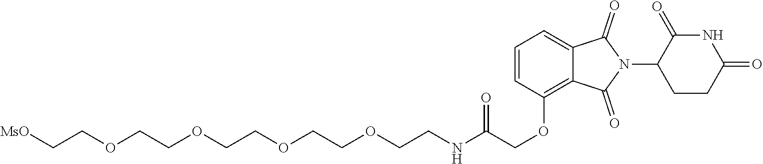 [SMILES: CS(=O)(=O)OCCOCCOCCOCCOCCNC(=O)COc1cccc2c1C(=O)N(C1CCC(=O)NC1=O)C2=O]

### Step 1: 2-((2-(2,6-Dioxopiperidin-3-yl)-1,3-dioxoisoindolin-4-yl)oxy)-N-(14-hydroxy-3,6,9,12-tetraoxatetradecyl)acetamide

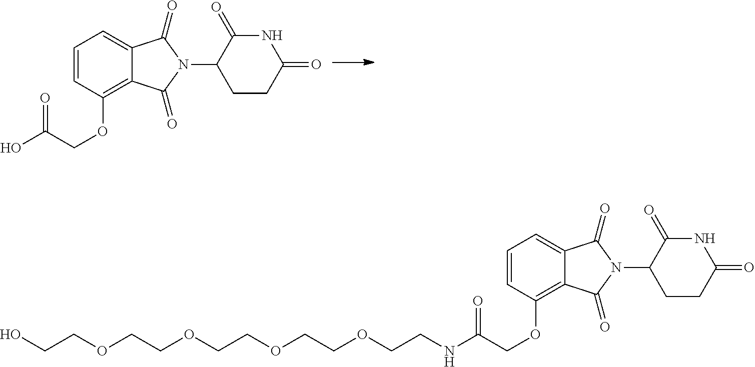 [SMILES: O=C(O)COc1cccc2c1C(=O)N(C1CCC(=O)NC1=O)C2=O ; O=C(COc1cccc2c1C(=O)N(C1CCC(=O)NC1=O)C2=O)NCCOCCOCCOCCOCCO]

A mixture of 2-((2-(2,6-dioxopiperidin-3-yl)-1,3-dioxoisoindolin-4-yl)oxy)acetic acid (300 mg, 0.90 mmol, 1.00 eq.), 14-amino-3,6,9,12-tetraoxatetradecan-1-ol (320 mg, 1.35 mmol, 1.50 eq.), HATU (513 mg, 1.35 mmol, 1.50 eq.) and DIPEA (348 mg, 2.69 mmol, 2.99 eq.) in DMF (6.0 mL) was stirred at 0° C. for 1 h. The reaction mixture was diluted with H₂O and extracted with DCM. The combined organic phase was washed with brine, dried over Na₂SO₄, concentrated to get crude title compound as a yellow oil, which was used for next step without further purification.

### Step 2: 1-((2-(2,6-Dioxopiperidin-3-yl)-1,3-dioxoisoindolin-4-yl)oxy)-2-oxo-6,9,12,15-tetraoxa-3-azaheptadecan-17-yl methanesulfonate

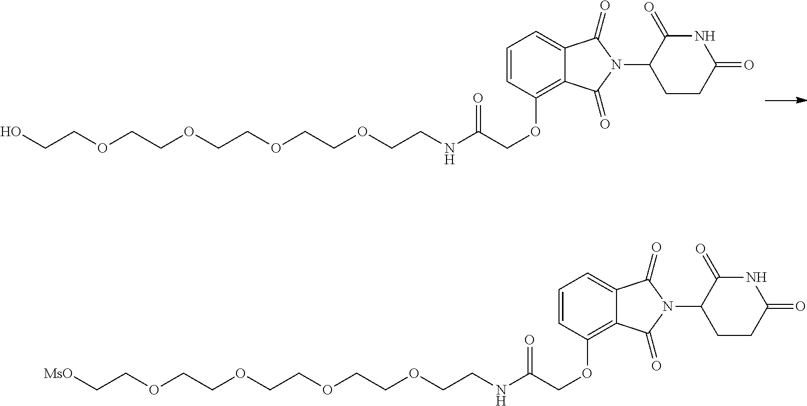 [SMILES: O=C(COc1cccc2c1C(=O)N(C1CCC(=O)NC1=O)C2=O)NCCOCCOCCOCCOCCO ; CS(=O)(=O)OCCOCCOCCOCCOCCNC(=O)COc1cccc2c1C(=O)N(C1CCC(=O)NC1=O)C2=O]

MsCl (271 mg, 2.37 mmol, 1.63 eq.) was added slowly to a stirred solution of 2-((2-(2,6-dioxopiperidin-3-yl)-1,3-dioxoisoindolin-4-yl)oxy)-N-(14-hydroxy-3,6,9,12-tetraoxatetradecyl)acetamide (800 mg, 1.45 mmol, 1.00 eq.) and TEA (479 mg, 4.73 mmol, 326 eq.) in DCM (8.0 mL) at 0° C. The reaction mixture was stirred at 0° C. for 1 h, diluted with H₂O and extracted with DCM. The combined organic phase was washed with brine, dried over Na₂SO₄, concentrated and purified by flash silica gel chromatography (DCM:MeOH=50:1) to give the title compound as a white solid.

### Reference 7

### Synthesis of 5-(3-(4-((4-aminopiperidin-1-yl)sulfonyl)phenoxy)azetidin-1-yl)-2-(2,6-dioxopiperidin-3-yl)isoindoline-1,3-dione

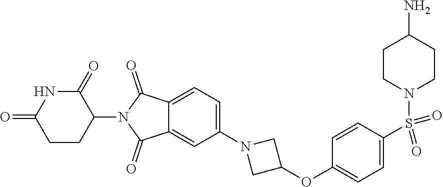 [SMILES: NC1CCN(S(=O)(=O)c2ccc(OC3CN(c4ccc5c(c4)C(=O)N(C4CCC(=O)NC4=O)C5=O)C3)cc2)CC1]

### Step 1: tert-Butyl (1-((4-fluorophenyl)sulfonyl)piperidin-4-yl)carbamate

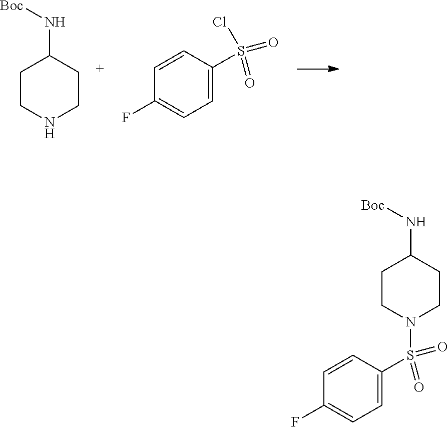 [SMILES: CC(C)(C)OC(=O)NC1CCNCC1 ; O=S(=O)(Cl)c1ccc(F)cc1 ; CC(C)(C)OC(=O)NC1CCN(S(=O)(=O)c2ccc(F)cc2)CC1]

A solution of 4-fluorobenzenesulfonyl chloride (2.6 g, 13.36 mmol, 1.07 eq.) in DCM (10.0 mL) was added dropwise to a stirred solution of tert-butyl piperidin-4-ylcarbamate (2.5 g, 12.48 mmol, 1.00 eq.) in DCM (10.0 mL) and TEA (5.2 mL) at 0° C. The resulting mixture was stirred at RT overnight, concentrated and diluted with DCM. The mixture was stirred at RT for 1 h and filtered to give the title compound as a white solid.

### Step 2: tert-Butyl (1-((4-((1-benzhydrylazetidin-3-yl)oxy)phenyl)sulfonyl)piperidin-4-yl)-carbamate

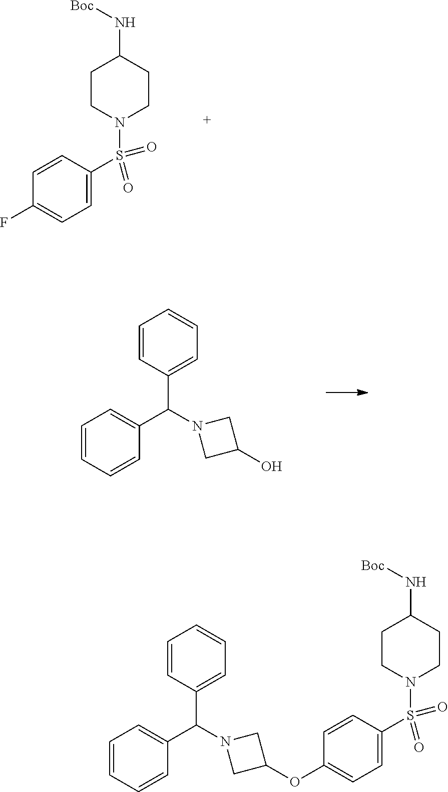 [SMILES: CC(C)(C)OC(=O)NC1CCN(S(=O)(=O)c2ccc(F)cc2)CC1 ; OC1CN(C(c2ccccc2)c2ccccc2)C1 ; CC(C)(C)OC(=O)NC1CCN(S(=O)(=O)c2ccc(OC3CN(C(c4ccccc4)c4ccccc4)C3)cc2)CC1]

To a stirred solution of 1-benzhydrylazetidin-3-ol (1.0 g, 4.18 mmol, 1.00 eq.) in THF (5.0 mL) was added NaH (60%, 251 mg, 6.28 mmol, 1.50 eq.) at 0° C. under N₂. The resulting mixture was stirred at RT for 15 min, then a solution of tert-butyl (1-((4-fluorophenyl)sulfonyl)piperidin-4-yl)carbamate (1.65 g, 4.60 mmol, 1.10 eq.) in THF (5.0 mL) was added slowly and the mixture was stirred at RT overnight. The mixture was diluted with H₂O, and then extracted with DCM. The combined organic layer was washed with aq. NaCl, dried over Na₂SO₄, filtered, and then concentrated. The residue was purified by silica gel flash column (PE:EA=3:1) to give the title compound as a white solid.

### Step 3: tert-Butyl (1-((4-(azetidin-3-yloxy)phenyl)sulfonyl)piperidin-4-yl)carbamate

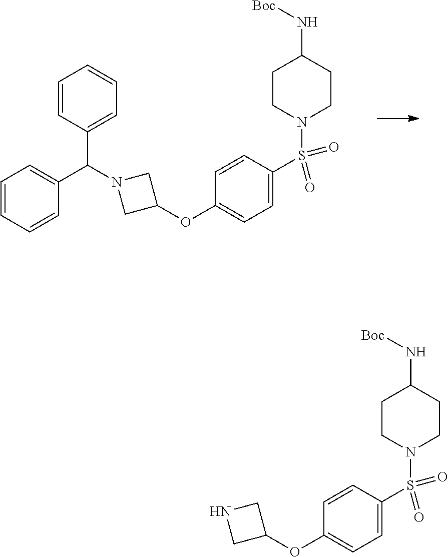 [SMILES: CC(C)(C)OC(=O)NC1CCN(S(=O)(=O)c2ccc(OC3CN(C(c4ccccc4)c4ccccc4)C3)cc2)CC1 ; CC(C)(C)OC(=O)NC1CCN(S(=O)(=O)c2ccc(OC3CNC3)cc2)CC1]

A mixture of tert-butyl (1-((4-((1-benzhydrylazetidin-3-yl)oxy)phenyl)sulfonyl)piperidin-4-yl)carbamate (500 mg, 0.87 mmol, 1.00 eq.), Pd(OH)₂ (300 mg, 20% on carbon) in THF (20.0 mL) was stirred at 50° C. under H₂ (50 psi) overnight. The mixture was cooled, filtrated, concentrated. The residue was purified by silica gel flash column (DCM:MeOH=10:1) to give the title compound as a white solid.

### Step 4: tert-Butyl (1-((4-((1-(2-(2,6-dioxopiperidin-3-yl)-1,3-dioxoisoindolin-5-yl)azetidin-3-yl)oxy)phenyl) sulfonyl)piperidin-4-yl)carbamate

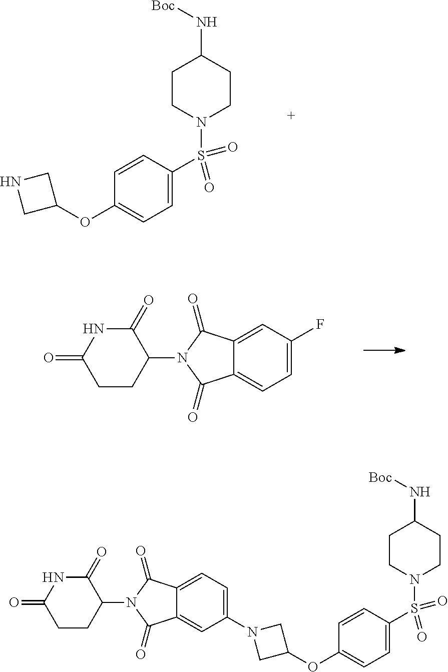 [SMILES: CC(C)(C)OC(=O)NC1CCN(S(=O)(=O)c2ccc(OC3CNC3)cc2)CC1 ; O=C1CCC(N2C(=O)c3ccc(F)cc3C2=O)C(=O)N1 ; CC(C)(C)OC(=O)NC1CCN(S(=O)(=O)c2ccc(OC3CN(c4ccc5c(c4)C(=O)N(C4CCC(=O)NC4=O)C5=O)C3)cc2)CC1]

A mixture of tert-butyl (1-((4-(-251-zetidine-3-yloxy)phenyl)sulfonyl)piperidin-4-yl) carbamate (100 mg, 0.24 mmol, 1.00 eq.), 2-(2,6-dioxopiperidin-3-yl)-5-fluoroisoindoline-1,3-dione (74 mg, 0.27 mmol, 1.13 eq.) and DIPEA (94 mg, 0.73 mmol, 3.04 eq.) in NMP (1.5 mL) was stirred at 140° C. 2 h under microwave irradiation. The mixture was cooled, diluted with water, extracted with DCM, and then concentrated. The residue was purified by silica gel flash column (PE:EA=1:1) to give the title product as a yellow solid.

### Step 5: 5-(3-(4-((4-Aminopiperidin-1-yl)sulfonyl)phenoxy)azetidin-1-yl)-2-(2,6-dioxopiperidin-3-yl)isoindoline-1,3-dione

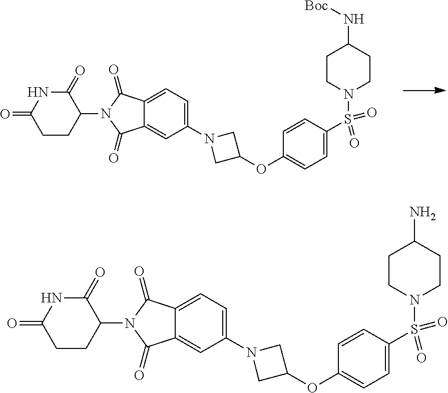 [SMILES: CC(C)(C)OC(=O)NC1CCN(S(=O)(=O)c2ccc(OC3CN(c4ccc5c(c4)C(=O)N(C4CCC(=O)NC4=O)C5=O)C3)cc2)CC1 ; NC1CCN(S(=O)(=O)c2ccc(OC3CN(c4ccc5c(c4)C(=O)N(C4CCC(=O)NC4=O)C5=O)C3)cc2)CC1]

A mixture of tert-butyl (1-((4-((1-(2-(2,6-dioxopiperidin-3-yl)-1,3-dioxo-isoindolin-5-yl)-251-zetidine-3-yl)oxy)phenyl)sulfonyl)piperidin-4-yl)carbamate (144 mg, 0.21 mmol, 1.00 eq.), TFA (1.0 mL) in DCM (4.0 mL) was stirred at RT for 2 h. The mixture was concentrated to give the title compound as a yellow oil, which was used for next step without further purification.

### Reference 8

### Synthesis of 5-((3-(4-((4-aminopiperidin-1-yl)sulfonyl)phenoxy)azetidin-1-yl)methyl)-2-(2,6-dioxo-piperidin-3-yl)isoindoline-1,3-dione

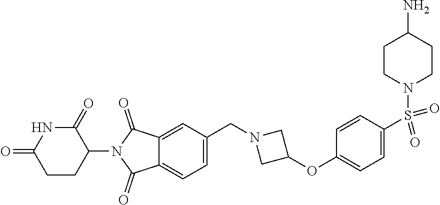 [SMILES: NC1CCN(S(=O)(=O)c2ccc(OC3CN(Cc4ccc5c(c4)C(=O)N(C4CCC(=O)NC4=O)C5=O)C3)cc2)CC1]

### Step 1: 5-(Bromomethyl)-2-(2,6-dioxopiperidin-3-yl)isoindoline-1,3-dione

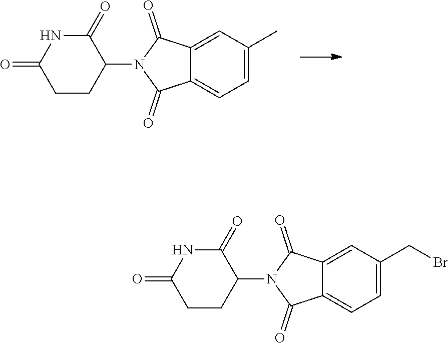 [SMILES: Cc1ccc2c(c1)C(=O)N(C1CCC(=O)NC1=O)C2=O ; O=C1CCC(N2C(=O)c3ccc(CBr)cc3C2=O)C(=O)N1]

NBS (196 mg, 1.10 mmol, 1.10 eq.) and AIBN (32.8 mg, 0.20 mmol, 0.20 eq.) were added to a stirred solution of 2-(2,6-dioxopiperidin-3-yl)-5-methylisoindoline-1,3-dione (272 mg, 1.00 mmol, 1.00 eq.) in MeCN (15.0 mL) and the mixture was stirred at 80° C. overnight under N₂. The mixture was cooled, and concentrated and the residue was purified by flash column chromatography (EA:PE=0-100%) gave the title compound as a white solid.

### Step 2: tert-Butyl (1-((4-((1-((2-(2,6-dioxopiperidin-3-yl)-1,3-dioxoisoindolin-5-yl)methyl) azetidin-3-yl)oxy)phenyl)sulfonyl)piperidin-4-yl)carbamate

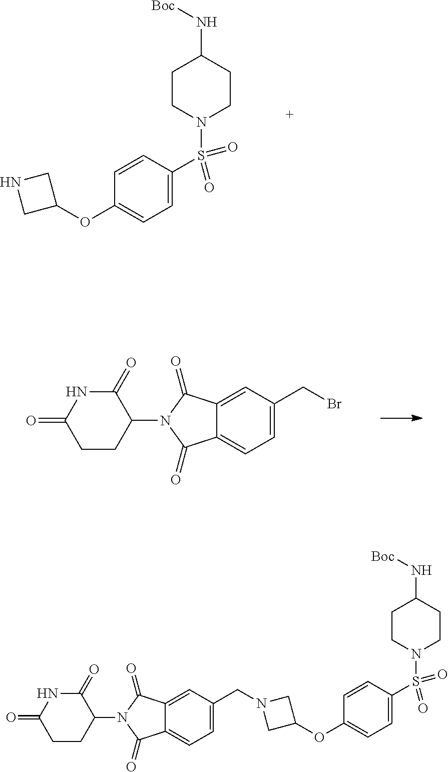 [SMILES: CC(C)(C)OC(=O)NC1CCN(S(=O)(=O)c2ccc(OC3CNC3)cc2)CC1 ; O=C1CCC(N2C(=O)c3ccc(CBr)cc3C2=O)C(=O)N1 ; CC(C)(C)OC(=O)NC1CCN(S(=O)(=O)c2ccc(OC3CN(Cc4ccc5c(c4)C(=O)N(C4CCC(=O)NC4=O)C5=O)C3)cc2)CC1]

A mixture of tert-butyl (1-((4-(azetidin-3-yloxy)phenyl)sulfonyl)piperidin-4-yl)carbamate (100 mg, 0.24 mmol, 1.00 eq., from Reference 7, Step 3), 5-(bromomethyl)-2-(2,6-dioxopiperidin-3-yl)isoindoline-1,3-dione (111 mg, 0.32 mmol, 1.33 eq.) and K₂CO₃ (67 mg, 0.48 mmol, 2.00 eq.) in MeCN (2.0 mL) was stirred at 80° C. overnight. The reaction mixture was cooled, concentrated and purified by silica gel flash column (DCM:MeOH=20:1) to give the title compound as a white solid.

### Step 3: 5-((3-(4-((4-Aminopiperidin-1-yl)sulfonyl)phenoxy)azetidin-1-yl)methyl)-2-(2,6-dioxo-piperidin-3-yl)isoindoline-1,3-dione

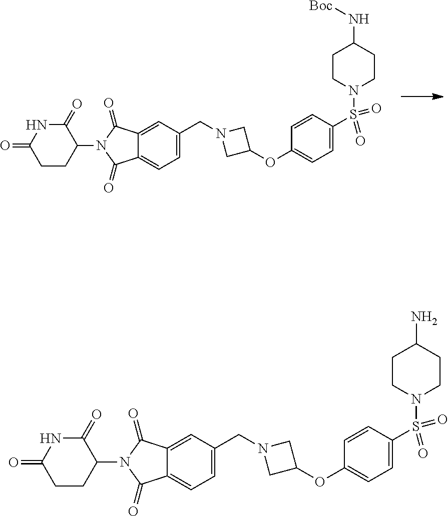 [SMILES: CC(C)(C)OC(=O)NC1CCN(S(=O)(=O)c2ccc(OC3CN(Cc4ccc5c(c4)C(=O)N(C4CCC(=O)NC4=O)C5=O)C3)cc2)CC1 ; NC1CCN(S(=O)(=O)c2ccc(OC3CN(Cc4ccc5c(c4)C(=O)N(C4CCC(=O)NC4=O)C5=O)C3)cc2)CC1]

A mixture of tert-butyl (1-((4-((1-((2-(2,6-dioxopiperidin-3-yl)-1,3-dioxoisoindolin-5-yl)methyl) azetidin-3-yl)oxy)phenyl)sulfonyl)piperidin-4-yl)carbamate (33 mg, 0.048 mmol, 1.00 eq.) and TFA (1.0 mL) in DCM (4.0 mL) was stirred at RT for 3 h. The mixture was concentrated to give the title compound as a yellow solid.

### Reference 9

### Synthesis of 4-amino-N-(2-(2-(2-((2-(2,6-dioxopiperidin-3-yl)-1,3-dioxoisoindolin-4-yl)amino)-ethoxy)ethoxy)ethyl)piperidine-1-sulfonamide

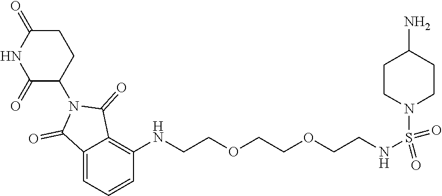 [SMILES: NC1CCN(S(=O)(=O)NCCOCCOCCNc2cccc3c2C(=O)N(C2CCC(=O)NC2=O)C3=O)CC1]

### Step 1: tert-Butyl(2-(2-(2-((2-(2,6-dioxopiperidin-3-yl)-1,3-dioxoisoindolin-4-yl)amino) ethoxy)-ethoxy)ethyl)carbamate

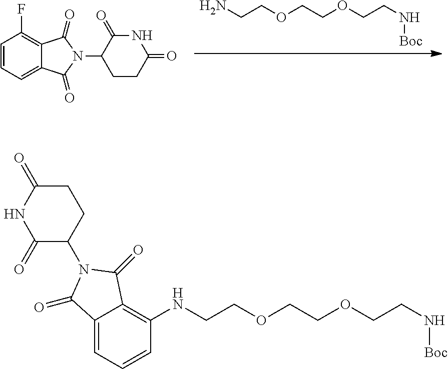 [SMILES: O=C1CCC(N2C(=O)c3cccc(F)c3C2=O)C(=O)N1 ; CC(C)(C)OC(=O)NCCOCCOCCN ; CC(C)(C)OC(=O)NCCOCCOCCNc1cccc2c1C(=O)N(C1CCC(=O)NC1=O)C2=O]

A mixture of 2-(2,6-dioxopiperidin-3-yl)-4-fluoroisoindoline-1,3-dione (100 mg, 0.36 mmol, 1.10 eq.), tert-butyl(2-(2-(2-aminoethoxy)ethoxy)ethyl)carbamate (81.7 mg, 0.33 mmol, 1.00 eq.), DIPEA (127 mg, 0.98 mmol, 2.97 eq.) in NMP (1.5 mL) was stirred at 140° C. under microwave for 2 h. The mixture was cooled and diluted with ethyl acetate, and then washed with water, brine, dried over Na₂SO₄, and concentrated. The residue was purified by silica gel chromatography (EA:PE=1:3) to give the title compound as a yellow oil.

### Step 2: 4-((2-(2-(2-Aminoethoxy)ethoxy)ethyl)amino)-2-(2,6-dioxopiperidin-3-yl)isoindoline-1,3-dione

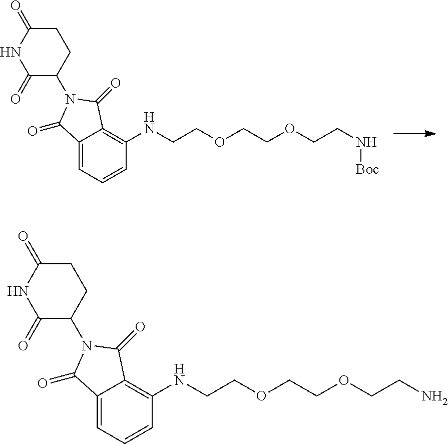 [SMILES: CC(C)(C)OC(=O)NCCOCCOCCNc1cccc2c1C(=O)N(C1CCC(=O)NC1=O)C2=O ; NCCOCCOCCNc1cccc2c1C(=O)N(C1CCC(=O)NC1=O)C2=O]

A mixture of tert-butyl(2-(2-(2-((2-(2,6-dioxopiperidin-3-yl)-1,3-dioxo-isoindolin-4-yl)amino) ethoxy) ethoxy)ethyl)carbamate (180 mg, 0.36 mmol, 1.00 eq.), TFA (0.5 mL) in DCM (2.0 mL) was stirred at RT for 2 h. The mixture was concentrated to give title compound as a yellow oil, which was used for next step without further purification.

### Step 3: tert-Butyl (1-(chlorosulfonyl)piperidin-4-yl)carbamate

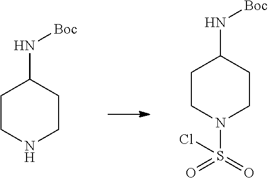 [SMILES: CC(C)(C)OC(=O)NC1CCNCC1 ; CC(C)(C)OC(=O)NC1CCN(S(=O)(=O)Cl)CC1]

Sulfuryl dichloride (81 mg, 0.60 mmol, 1.20 eq.) was added to a stirred solution of tert-butyl piperidin-4-ylcarbamate (100 mg, 0.50 mmol, 1.00 eq.) and TEA (76 mg, 0.75 mmol, 1.50 eq.) in DCM (2.0 mL) at 0° C. and the mixture was stirred at 0° C. for 3 h. The mixture was diluted with water, and then extracted with DCM. The organic layer was washed with brine, dried over Na₂SO₄, and then concentrated to give the title compound as a white solid, which was used for next step directly.

### Step 4: tert-Butyl (1-(N-(2-(2-(2-((2-(2,6-dioxopiperidin-3-yl)-1,3-dioxoisoindolin-4-yl)amino)-ethoxy)ethoxy)ethyl)sulfamoyl)piperidin-4-yl)carbamate

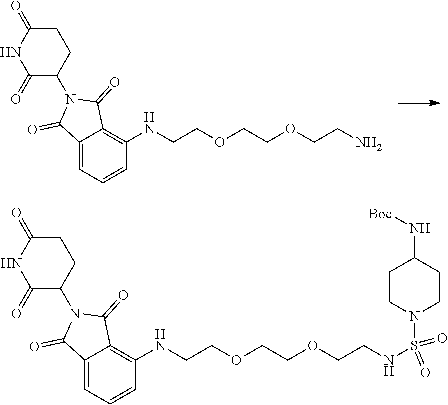 [SMILES: NCCOCCOCCNc1cccc2c1C(=O)N(C1CCC(=O)NC1=O)C2=O ; CC(C)(C)OC(=O)NC1CCN(S(=O)(=O)NCCOCCOCCNc2cccc3c2C(=O)N(C2CCC(=O)NC2=O)C3=O)CC1]

To a stirred solution of 4-((2-(2-(2-aminoethoxy)ethoxy)ethyl)amino)-2-(2,6-dioxo-piperidin-3-yl)isoindoline-1,3-dione (70 mg, 0.17 mmol, 1.00 eq.) and tert-butyl (1-(chloro-sulfonyl)piperidin-4-yl)carbamate (51.9 mg, 0.17 mmol, 1.00 eq.) in DCM (2.0 mL) was added TEA (52.4 mg, 0.52 mmol, 3.00 eq.). The mixture was stirred at 35° C. overnight, and then concentrated. The residue was purified by silica gel chromatography (DCM:MeOH=30:1) to give the title compound as a yellow oil.

### Step 5: 4-Amino-N-(2-(2-(2-((2-(2,6-dioxopiperidin-3-yl)-1,3-dioxoisoindolin-4-yl)amino)-ethoxy)ethoxy)ethyl)piperidine-1-sulfonamide

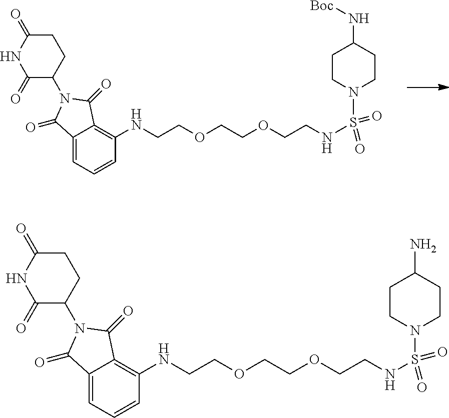 [SMILES: CC(C)(C)OC(=O)NC1CCN(S(=O)(=O)NCCOCCOCCNc2cccc3c2C(=O)N(C2CCC(=O)NC2=O)C3=O)CC1 ; NC1CCN(S(=O)(=O)NCCOCCOCCNc2cccc3c2C(=O)N(C2CCC(=O)NC2=O)C3=O)CC1]

A mixture of tert-butyl (1-(N-(2-(2-(2-((2-(2,6-dioxopiperidin-3-yl)-1,3-dioxoisoindolin-4-yl)amino) ethoxy)ethoxy)ethyl)sulfamoyl)piperidin-4-yl)carbamate (60 mg, 0.090 mmol, 1.00 eq.) in DCM (2.0 mL) and TFA (0.5 mL) was stirred at RT for 2 h. The mixture was concentrated to give the title compound as a yellow oil.

### Reference 10

### Synthesis of 4-amino-N-(3-(3-(2-(2,6-dioxopiperidin-3-yl)-1,3-dioxoisoindolin-4-yl)propoxy)-propyl)-N-methylpiperidine-1-sulfonamide

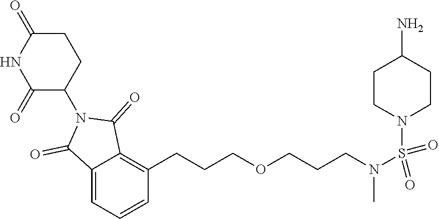 [SMILES: CN(CCCOCCCc1cccc2c1C(=O)N(C1CCC(=O)NC1=O)C2=O)S(=O)(=O)N1CCC(N)CC1]

### Step 1: 4-Bromo-2-(2,6-dioxopiperidin-3-yl)isoindoline-1,3-dione

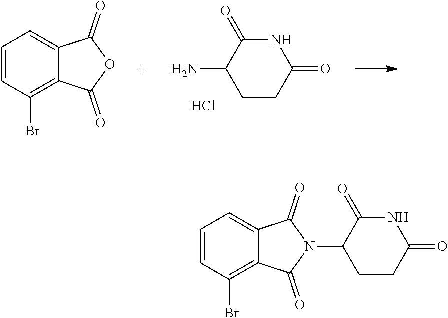 [SMILES: O=C1OC(=O)c2c(Br)cccc21 ; NC1CCC(=O)NC1=O ; Cl ; O=C1CCC(N2C(=O)c3cccc(Br)c3C2=O)C(=O)N1]

A mixture of 4-bromoisobenzofuran-1,3-dione (22.8 g, 100.44 mmol, 1.00 eq.), 3-aminopiperidine-2,6-dione (18.0 g, 109.36 mmol, HCl, 1.09 eq.) and KOAc (29.4 g, 299.54 mmol, 2.98 eq.) in HOAc (200.0 mL) was stirred at 90° C. for 16 h. The reaction mixture was cooled, diluted with ice water and then stirred at 0° C. for 1 h. The mixture was filtered and the filter cake was dried in vacuo to give the title compound as gray solid.

### Step 2: tert-Butyl methyl (3-(prop-2-yn-1-yloxy)propyl)carbamate

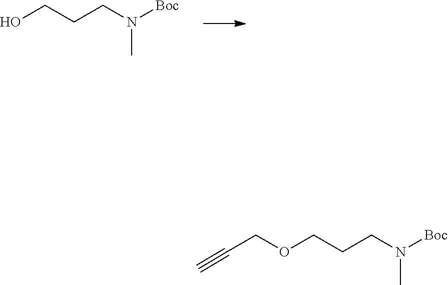 [SMILES: CN(CCCO)C(=O)OC(C)(C)C ; C#CCOCCCN(C)C(=O)OC(C)(C)C]

A mixture of tert-butyl (3-hydroxypropyl)(methyl)carbamate (3.0 g, 15.85 mmol, 1.00 eq.) in DCM (50.0 mL), 3-bromoprop-1-yne (3.0 g, 25.22 mmol, 1.59 eq.), 40% aqueous NaOH (30.0 mL) and tetrabutylammonium hydrogen sulfate (270 mg, 0.80 mmol, 0.050 eq.) was stirred at RT overnight under N₂. The mixture was diluted with water, and then extracted with DCM. The organic layer was washed with water, brine, dried over Na₂SO₄, concentrated. The residue was purified by flash column chromatography (EA:PE=0 to 100%) to give the title compound as a yellow oil.

### Step 3: tert-Butyl (3-((3-(2-(2,6-dioxopiperidin-3-yl)-1,3-dioxoisoindolin-4-yl)prop-2-yn-1-yl)oxy)propyl)(methyl)carbamate

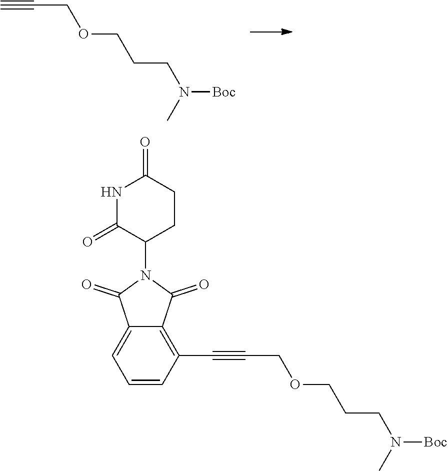

A mixture of 4-bromo-2-(2,6-dioxopiperidin-3-yl)isoindoline-1,3-dione (1.38 g, 4.09 mmol, 1.00 eq.), tert-butyl methyl (3-(prop-2-yn-1-yloxy)-propyl)carbamate (1.4 g, 6.16 mmol, 1.51 eq.), CuI (78 mg, 0.41 mmol, 0.10 eq.), TEA (7.5 g, 74.12 mmol, 18.12 eq.) and Pd(PPh₃)₂Cl₂ (288 mg, 0.41 mmol, 0.10 eq.) in DMF (15.0 mL) was stirred at 80° C. for 2 h under N₂. The mixture was cooled, diluted with water and then extracted with ethyl acetate. The organic layer was washed with water, brine, dried over Na₂SO₄, and concentrated. Purification by flash column chromatography (EA:PE=0 to 100%) to give the title compound as a yellow oil.

### Step 4: tert-Butyl (3-(3-(2-(2,6-dioxopiperidin-3-yl)-1,3-dioxoisoindolin-4-yl)propoxy)propyl)-(methyl)carbamate

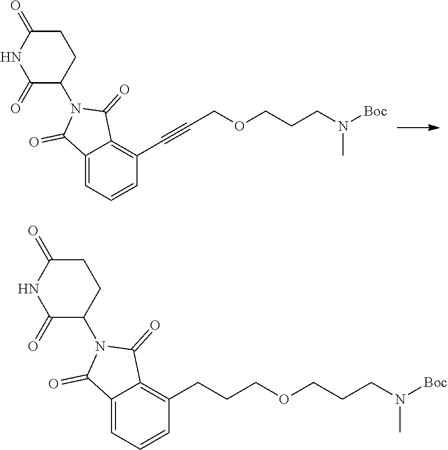 [SMILES: CN(CCCOCC#Cc1cccc2c1C(=O)N(C1CCC(=O)NC1=O)C2=O)C(=O)OC(C)(C)C ; CN(CCCOCCCc1cccc2c1C(=O)N(C1CCC(=O)NC1=O)C2=O)C(=O)OC(C)(C)C]

A mixture of tert-butyl (3-((3-(2-(2,6-dioxopiperidin-3-yl)-1,3-dioxoisoindolin-4-yl)prop-2-yn-1-yl)oxy)propyl)(methyl)carbamate (1.86 g, 3.85 mmol, 1.00 eq.), Pd(OH)₂/C (0.93 g, 50% w/w) in THF (50.0 mL) was stirred at RT overnight under H₂ atmosphere. The reaction mixture was filtered, concentrated and the residue was purified by flash chromatography (EA:PE=0 to 100%) to give the title compound as a yellow oil.

### Step 5: 2-(2,6-Dioxopiperidin-3-yl)-4-(3-(3-(methylamino)propoxy)propyl)isoindoline-1,3-dione

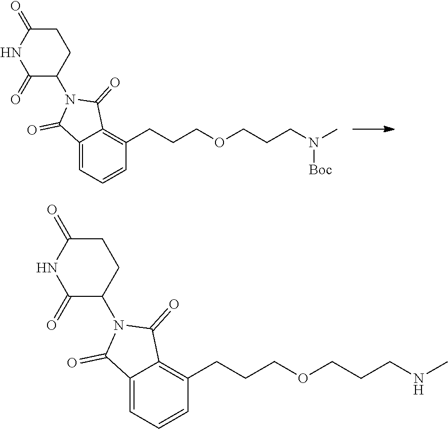 [SMILES: CN(CCCOCCCc1cccc2c1C(=O)N(C1CCC(=O)NC1=O)C2=O)C(=O)OC(C)(C)C ; CNCCCOCCCc1cccc2c1C(=O)N(C1CCC(=O)NC1=O)C2=O]

A mixture of tert-butyl (3-(3-(2-(2,6-dioxopiperidin-3-yl)-1,3-dioxoisoindolin-4-yl)propoxy)propyl)(methyl)carbamate (1.45 g, 2.97 mmol, 1.00 eq.), TFA (1.0 mL) in DCM (10.0 mL) was stirred at RT for 2 h under N₂. The mixture was concentrated and adjusted pH to 9 using aqueous Na₂CO₃, and then the mixture was extracted with DCM. The organic layer was washed with water, brine, dried over Na₂SO₄, and then concentrated to give the title compound as a yellow oil.

### Step 6: tert-Butyl (1-(N-(3-(3-(2-(2,6-dioxopiperidin-3-yl)-1,3-dioxoisoindolin-4-yl)propoxy)-propyl)-N-methylsulfamoyl)piperidin-4-yl)carbamate

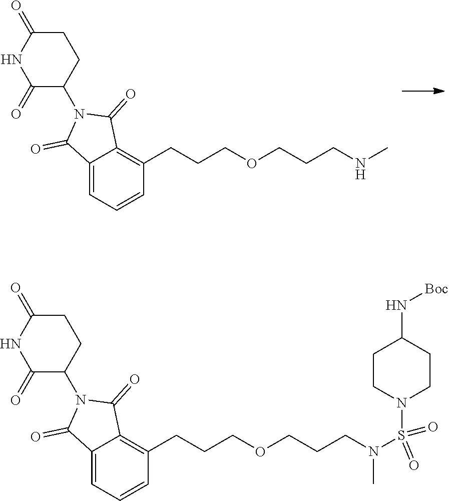 [SMILES: CNCCCOCCCc1cccc2c1C(=O)N(C1CCC(=O)NC1=O)C2=O ; CN(CCCOCCCc1cccc2c1C(=O)N(C1CCC(=O)NC1=O)C2=O)S(=O)(=O)N1CCC(NC(=O)OC(C)(C)C)CC1]

A mixture of 2-(2,6-dioxopiperidin-3-yl)-4-(3-(3-(methylamino) propoxy)-propyl)isoindoline-1,3-dione (150 mg, 0.39 mmol, 1.00 eq.), tert-butyl (1-(chlorosulfonyl)-piperidin-4-yl)carbamate (173 mg, 0.58 mmol, 1.49 eq.) and TEA (118 mg, 1.17 mmol, 3.00 eq.) in DCM (2.0 mL) was stirred at 40° C. overnight under N₂. The mixture was cooled, diluted with water and then extracted with ethyl acetate. The organic layer was washed with water, brine, dried over Na₂SO₄, concentrated to give the title compound as a yellow solid.

### Step 7: 4-Amino-N-(3-(3-(2-(2,6-dioxopiperidin-3-yl)-1,3-dioxoisoindolin-4-yl)propoxy)propyl)-N-methylpiperidine-1-sulfonamide

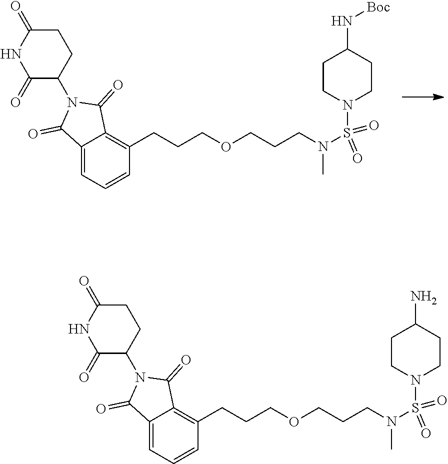 [SMILES: CN(CCCOCCCc1cccc2c1C(=O)N(C1CCC(=O)NC1=O)C2=O)S(=O)(=O)N1CCC(NC(=O)OC(C)(C)C)CC1 ; CN(CCCOCCCc1cccc2c1C(=O)N(C1CCC(=O)NC1=O)C2=O)S(=O)(=O)N1CCC(N)CC1]

To a stirred solution of tert-butyl (1-(N-(3-(3-(2-(2,6-dioxopiperidin-3-yl)-1,3-dioxoisoindolin-4-yl)propoxy)propyl)-N-methylsulfamoyl)piperidin-4-yl)carbamate (200 mg, 0.31 mmol, 1.00 eq.) in DCM (2.0 mL) was added TFA (0.5 mL). The resulting mixture was stirred at RT for 3 h under N₂, concentrated and adjusted pH to 9 using aqueous Na₂CO₃, and then extracted with DCM. The organic layer was washed with water, brine, dried over Na₂SO₄, concentrated to give the title compound as a yellow oil, which was used for next step without further purification.

### Reference 11

### Synthesis of 4-(3-(2-(2-aminoethoxy)ethoxy)propyl)-2-(2,6-dioxopiperidin-3-yl)isoindoline-1,3-dione

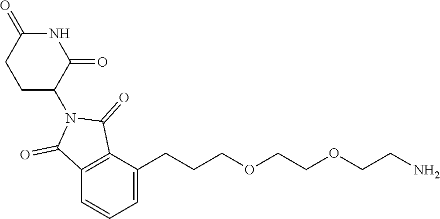 [SMILES: NCCOCCOCCCc1cccc2c1C(=O)N(C1CCC(=O)NC1=O)C2=O]

### Step 1: tert-Butyl(2-(2-(prop-2-yn-1-yloxy) ethoxy)ethyl)carbamate

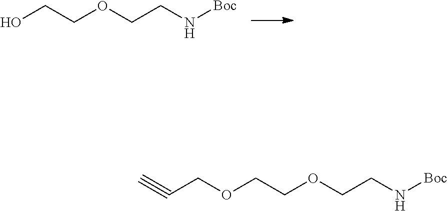 [SMILES: CC(C)(C)OC(=O)NCCOCCO ; C#CCOCCOCCNC(=O)OC(C)(C)C]

NaH (1.2 g, 60%, 30.00 mmol, 1.50 eq.) was added to a stirred solution of tert-butyl(2-(2-hydroxyethoxy)ethyl)carbamate (4.1 g, 19.98 mmol, 1.00 eq.) in THF (50.0 mL) in portions at 0° C. and the mixture was stirred for 1 h. Then 3-bromoprop-1-yne (2.83 g, 23.79 mmol, 1.19 eq.) was added at 0° C. The reaction mixture was warmed to RT and stirred for 16 h, poured into water and extracted with DCM. The organic layer was washed with water, brine, dried over Na₂SO₄, concentrated and purified by flash column chromatography (EA:PE=0 to 100%) to give the title compound as a yellow oil.

### Step 2: 4-Amino-N-(2-(2-(3-(2-(2,6-dioxopiperidin-3-yl)-1,3-dioxoisoindolin-4-yl)propoxy)ethoxy)ethyl)piperidine-1-sulfonamide

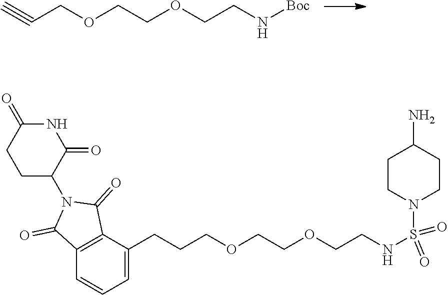 [SMILES: C#CCOCCOCCNC(=O)OC(C)(C)C ; NC1CCN(S(=O)(=O)NCCOCCOCCCc2cccc3c2C(=O)N(C2CCC(=O)NC2=O)C3=O)CC1]

Proceeding analogously as described in Reference 10, Step 3-7 above, but using tert-Butyl (2-(2-(prop-2-yn-1-yloxy) ethoxy)ethyl)carbamate provided the title compound.

### Reference 12

### Synthesis of 5-(3-(3-((4-aminopiperidin-1-yl)sulfonyl)phenoxy)azetidin-1-yl)-2-(2,6-dioxopiperidin-3-yl)isoindoline-1,3-dione

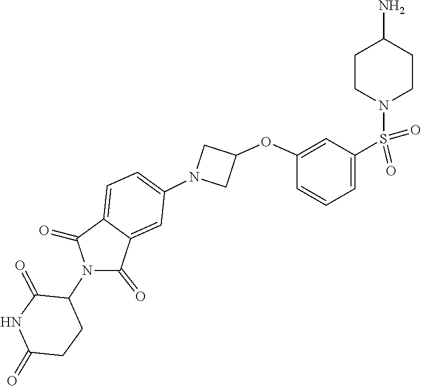 [SMILES: NC1CCN(S(=O)(=O)c2cccc(OC3CN(c4ccc5c(c4)C(=O)N(C4CCC(=O)NC4=O)C5=O)C3)c2)CC1]

### Step 1: Benzyl (1-((3-methoxyphenyl)sulfonyl)piperidin-4-yl)carbamate

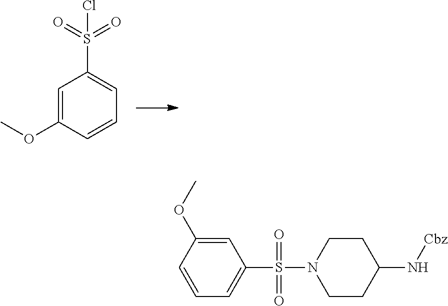 [SMILES: COc1cccc(S(=O)(=O)Cl)c1 ; COc1cccc(S(=O)(=O)N2CCC(NC(=O)OCc3ccccc3)CC2)c1]

A solution of 3-methoxybenzene-1-sulfonyl chloride (3.24 g, 15.68 mmol, 1.05 eq.) in DCM (20.0 mL) was added dropwise to a stirred solution of benzyl piperidin-4-ylcarbamate (3.5 g, 14.94 mmol, 1.00 eq.) and TEA (4.52 g, 44.82 mmol, 3.00 eq.) in DCM (50.0 mL) at 0° C. and the mixture was stirred at RT for 3 h. The mixture was diluted with DCM and the organic layer was washed with water and brine, dried over Na₂SO₄, and concentrated. The residue was purified by silica gel chromatography (EA:PE=1:3) gave the title compound as a white solid.

### Step 2: 3-((4-Aminopiperidin-1-yl)sulfonyl)phenol

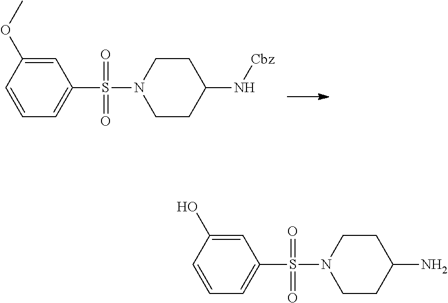 [SMILES: COc1cccc(S(=O)(=O)N2CCC(NC(=O)OCc3ccccc3)CC2)c1 ; NC1CCN(S(=O)(=O)c2cccc(O)c2)CC1]

The solution of benzyl (1-((3-methoxyphenyl)sulfonyl)piperidin-4-yl)carbamate (3.5 g, 8.66 mmol, 1.00 eq.) in CF₃SO₃H (20.0 mL) was stirred under N₂ at 100° C. for 3 h. The reaction mixture was cooled and concentrated to give the title compound as a brown oil, which was used for next step without further purification.

### Step 3: tert-Butyl (1-((3-hydroxyphenyl)sulfonyl)piperidin-4-yl)carbamate

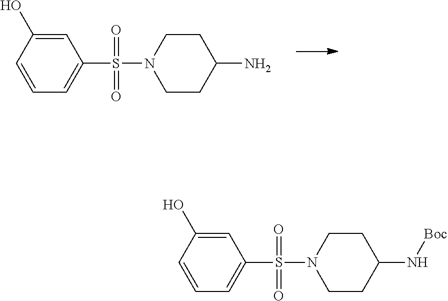 [SMILES: NC1CCN(S(=O)(=O)c2cccc(O)c2)CC1 ; CC(C)(C)OC(=O)NC1CCN(S(=O)(=O)c2cccc(O)c2)CC1]

A solution of (Boc) 20 (852 mg, 3.91 mmol, 1.00 eq.) in DCM (5.0 mL) was added to a stirred solution of 3-((4-aminopiperidin-1-yl)sulfonyl)phenol (1.0 g, 3.91 mmol, 1.00 eq.) in DCM (20.0 mL) and TEA (1.18 g, 11.73 mmol, 3.00 eq.) at 0° C. The mixture was stirred at RT for 2 h, diluted with DCM and the organic layer was washed with water, brine, dried over Na₂SO₄, and concentrated. The residue was purified by flash silica gel chromatography (ACN/water=(35%-75%)) to give the title compound as a white solid.

### Step 4: 1-Benzhydrylazetidin-3-yl methanesulfonate

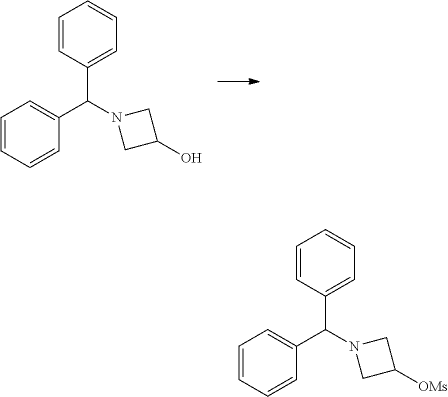 [SMILES: OC1CN(C(c2ccccc2)c2ccccc2)C1 ; CS(=O)(=O)OC1CN(C(c2ccccc2)c2ccccc2)C1]

To a stirred solution of 1-benzhydrylazetidin-3-ol (500 mg, 2.09 mmol, 1.00 eq.) in DCM (10.0 mL) was added TEA (633 mg, 6.27 mmol, 3.00 eq.) and MsCl (479 mg, 4.18 mmol, 2.00 eq.) at 0° C. The mixture was stirred at RT overnight, diluted with DCM and then washed with water. The organic layer was washed with brine, dried over Na₂SO₄, concentrated and then purified by silica gel chromatography (EA:PE=1:3) to give the title compound as a white solid.

### Step 5: tert-Butyl (1-((3-((1-benzhydrylazetidin-3-yl)oxy)phenyl)sulfonyl)piperidin-4-yl)-carbamate

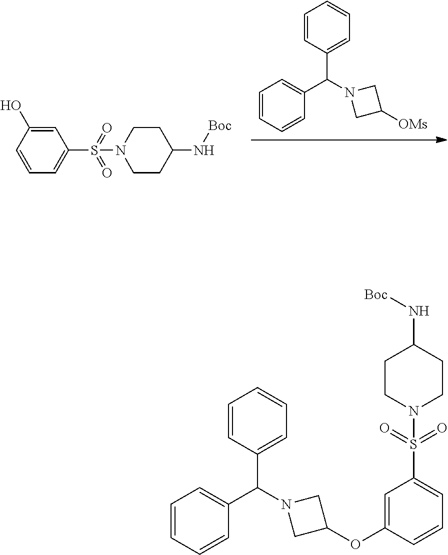 [SMILES: CC(C)(C)OC(=O)NC1CCN(S(=O)(=O)c2cccc(O)c2)CC1 ; CS(=O)(=O)OC1CN(C(c2ccccc2)c2ccccc2)C1 ; CC(C)(C)OC(=O)NC1CCN(S(=O)(=O)c2cccc(OC3CN(C(c4ccccc4)c4ccccc4)C3)c2)CC1]

A mixture of tert-butyl (1-((3-hydroxyphenyl)sulfonyl)piperidin-4-yl)carbamate (533 mg, 1.49 mmol, 1.00 eq.), 1-benzhydrylazetidin-3-yl methanesulfonate (570 mg, 1.79 mmol, 1.20 eq.), Cs₂CO₃ (1.46 g, 4.49 mmol, 3.00 eq.) in DMSO (10.0 mL) was stirred at 90° C. under N₂ for 3 h. The mixture was cooled, diluted with EtOAc and the organic layer was washed with brine, dried over Na₂SO₄, concentrated. The residue was purified by silica gel chromatography (EA:PE=1:3) to give the title compound as a pale yellow solid.

### Step 6: tert-Butyl (1-((3-(azetidin-3-yloxy)phenyl)sulfonyl)piperidin-4-yl)carbamate

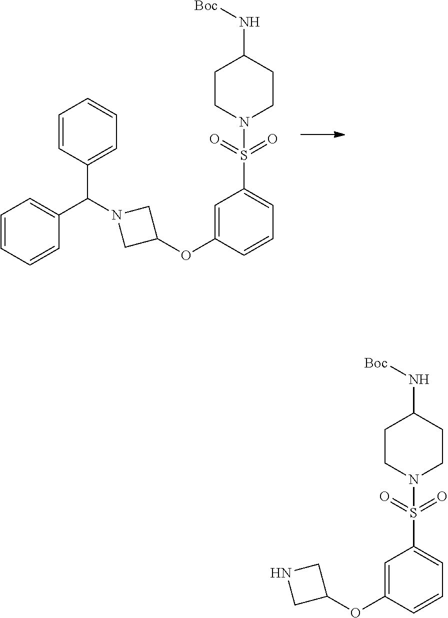 [SMILES: CC(C)(C)OC(=O)NC1CCN(S(=O)(=O)c2cccc(OC3CN(C(c4ccccc4)c4ccccc4)C3)c2)CC1 ; CC(C)(C)OC(=O)NC1CCN(S(=O)(=O)c2cccc(OC3CNC3)c2)CC1]

To a stirred solution of tert-butyl (1-((3-((1-benzhydrylazetidin-3-yl)oxy)phenyl)sulfonyl)-piperidin-4-yl)carbamate (400 mg, 0.69 mmol, 1.00 eq.) in MeOH (15.0 mL) were added Pd(OH)₂/C (20 wt. %, 250 mg) and AcOH (0.5 mL) at RT. The resulting mixture was stirred at 50° C. under H₂ (50 psi) overnight. The reaction mixture was cooled and filtered, and the filtrate was concentrated. The residue was purified by silica gel chromatography (MeOH:DCM=1:15) to give the title compound as a white solid.

### Step 7: tert-Butyl (1-((3-((1-(2-(2,6-dioxopiperidin-3-yl)-1,3-dioxoisoindolin-5-yl)azetidin-3-yl)-oxy)phenyl)sulfonyl)piperidin-4-yl)carbamate

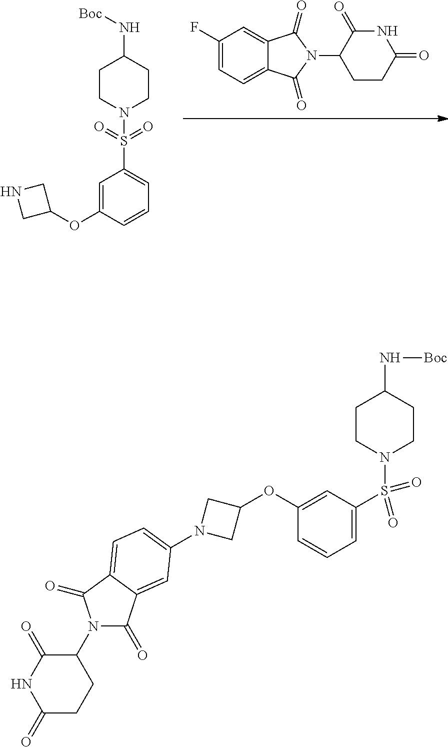 [SMILES: CC(C)(C)OC(=O)NC1CCN(S(=O)(=O)c2cccc(OC3CNC3)c2)CC1 ; O=C1CCC(N2C(=O)c3ccc(F)cc3C2=O)C(=O)N1 ; CC(C)(C)OC(=O)NC1CCN(S(=O)(=O)c2cccc(OC3CN(c4ccc5c(c4)C(=O)N(C4CCC(=O)NC4=O)C5=O)C3)c2)CC1]

Proceeding analogously as described in Reference 7, Step 4 above, but using tert-butyl (1-((3-(azetidin-3-yloxy)phenyl)sulfonyl)piperidin-4-yl)carbamate and 2-(2,6-dioxopiperidin-3-yl)-5-fluoroisoindoline-1,3-dione provided the title compound.

### Step 8: 5-(3-(3-((4-Aminopiperidin-1-yl)sulfonyl)phenoxy)azetidin-1-yl)-2-(2,6-dioxopiperidin-3-yl)isoindoline-1,3-dione

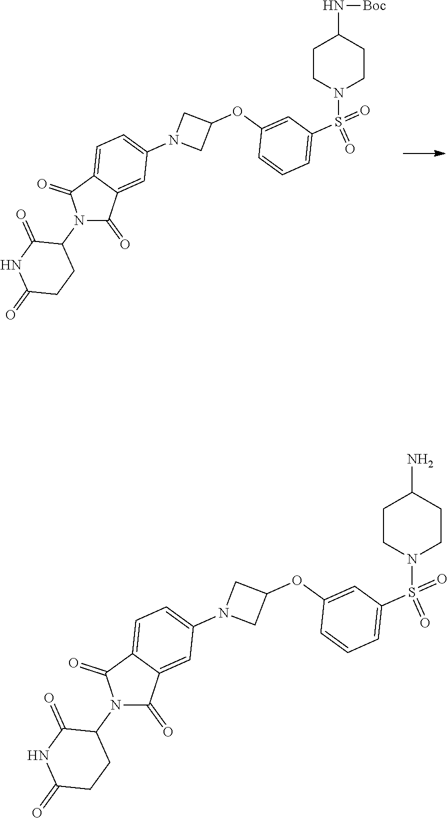 [SMILES: CC(C)(C)OC(=O)NC1CCN(S(=O)(=O)c2cccc(OC3CN(c4ccc5c(c4)C(=O)N(C4CCC(=O)NC4=O)C5=O)C3)c2)CC1 ; NC1CCN(S(=O)(=O)c2cccc(OC3CN(c4ccc5c(c4)C(=O)N(C4CCC(=O)NC4=O)C5=O)C3)c2)CC1]

Proceeding analogously as described in Reference 7, Step 5 above, but using tert-butyl (1-((3-((1-(2-(2,6-dioxopiperidin-3-yl)-1,3-dioxoisoindolin-5-yl)azetidin-3-yl)oxy)phenyl)-sulfonyl)piperidin-4-yl)carbamate provided the title compound.

### Reference 13

### Synthesis of 3-(4-(3-((1-((1-((4-aminopiperidin-1-yl)sulfonyl)piperidin-4-yl)methyl)piperidin-4-yl)-oxy)prop-1-yn-1-yl)-3-methyl-2-oxo-2,3-dihydro-1H-benzo[d]imidazol-1-yl)piperidine-2,6-dione

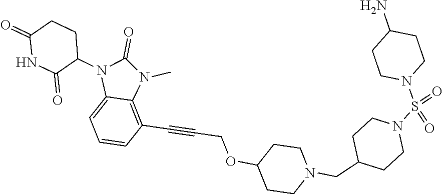 [SMILES: Cn1c(=O)n(C2CCC(=O)NC2=O)c2cccc(C#CCOC3CCN(CC4CCN(S(=O)(=O)N5CCC(N)CC5)CC4)CC3)c21]

### Step 1: tert-Butyl 4-((4-((3-(1-(2,6-dioxopiperidin-3-yl)-3-methyl-2-oxo-2,3-dihydro-1H-benzo[d]imidazol-4-yl)prop-2-yn-1-yl)oxy)piperidin-1-yl)methyl)piperidine-1-carboxylate

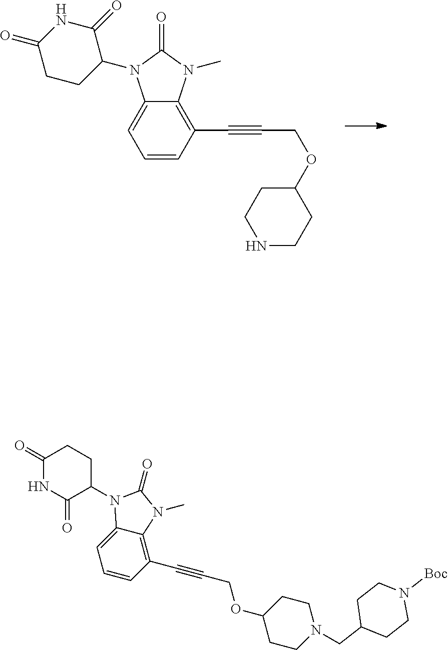 [SMILES: Cn1c(=O)n(C2CCC(=O)NC2=O)c2cccc(C#CCOC3CCNCC3)c21 ; Cn1c(=O)n(C2CCC(=O)NC2=O)c2cccc(C#CCOC3CCN(CC4CCN(C(=O)OC(C)(C)C)CC4)CC3)c21]

A mixture of 3-(3-methyl-2-oxo-4-(3-(piperidin-4-yloxy)prop-1-yn-1-yl)-2,3-dihydro-1H-benzo[d]imidazol-1-yl)piperidine-2,6-dione (300 mg, 0.76 mmol, 1.00 eq., prepared by proceeding as described in Reference 11, Steps 1 and 2 above), tert-butyl 4-(bromomethyl)piperidine-1-carboxylate (421 mg, 1.51 mmol, 2.00 eq.), NaI (114 mg, 0.76 mmol, 1.00 eq.), K₂CO₃ (634.8 mg, 4.59 mmol, 6.00 eq.) in ACN (5.0 mL) was stirred at 70° C. overnight. The reaction mixture was cooled, concentrated and then purified with chromatograph on silica gel (DCM/MeOH=20/1) to give the title compound as a yellow solid.

### Step 2: 3-(3-Methyl-2-oxo-4-(3-((1-(piperidin-4-ylmethyl)piperidin-4-yl)oxy)prop-1-yn-1-yl)-2,3-dihydro-1H-benzo[d]imidazol-1-yl)piperidine-2,6-dione

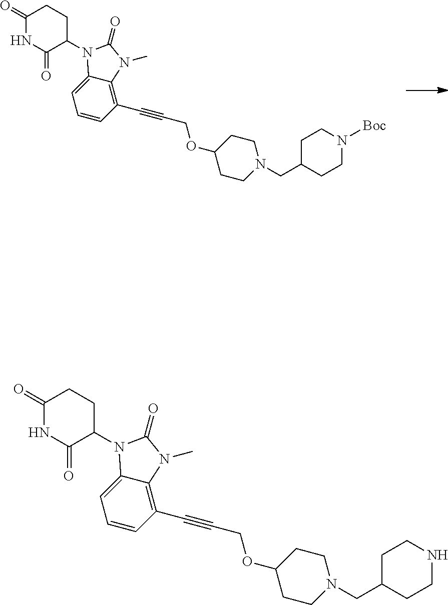 [SMILES: Cn1c(=O)n(C2CCC(=O)NC2=O)c2cccc(C#CCOC3CCN(CC4CCN(C(=O)OC(C)(C)C)CC4)CC3)c21 ; Cn1c(=O)n(C2CCC(=O)NC2=O)c2cccc(C#CCOC3CCN(CC4CCNCC4)CC3)c21]

Proceeding analogously as described in Reference 10, Step 5 above, but using tert-butyl 4-((4-((3-(1-(2,6-dioxopiperidin-3-yl)-3-methyl-2-oxo-2,3-dihydro-1H-benzo[d]imidazol-4-yl)prop-2-yn-1-yl)oxy)piperidin-1-yl)methyl)piperidine-1-carboxylate provided the title compound.

### Step 3: tert-Butyl (1-((4-((4-((3-(1-(2,6-dioxopiperidin-3-yl)-3-methyl-2-oxo-2,3-dihydro-1H-benzo[d]imidazol-4-yl)prop-2-yn-1-yl)oxy)piperidin-1-yl)methyl)piperidin-1-yl)sulfonyl)piperidin-4-yl)carbamate

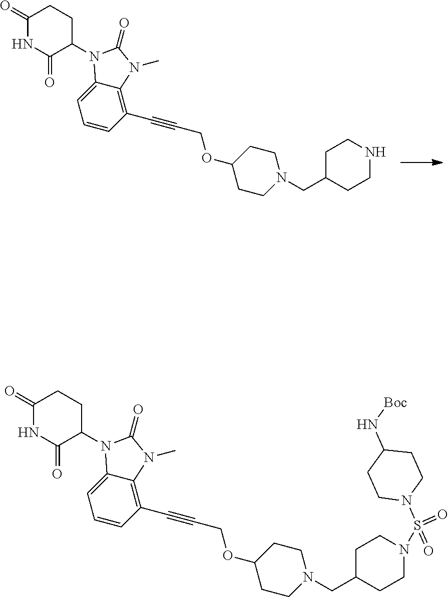 [SMILES: Cn1c(=O)n(C2CCC(=O)NC2=O)c2cccc(C#CCOC3CCN(CC4CCNCC4)CC3)c21 ; Cn1c(=O)n(C2CCC(=O)NC2=O)c2cccc(C#CCOC3CCN(CC4CCN(S(=O)(=O)N5CCC(NC(=O)OC(C)(C)C)CC5)CC4)CC3)c21]

Proceeding analogously as described in Reference 10, Step 6 above, but using 3-(3-methyl-2-oxo-4-(3-((1-(piperidin-4-ylmethyl)piperidin-4-yl)oxy)prop-1-yn-1-yl)-2,3-dihydro-1H-benzo[d]imidazol-1-yl)piperidine-2,6-dione and tert-butyl (1-(chlorosulfonyl)piperidin-4-yl)carbamate provided the title compound.

### Step 4: 3-(4-(3-((1-((1-((4-Aminopiperidin-1-yl)sulfonyl)piperidin-4-yl)methyl)piperidin-4-yl)-oxy)prop-1-yn-1-yl)-3-methyl-2-oxo-2,3-dihydro-1H-benzo[d]imidazol-1-yl)piperidine-2,6-dione

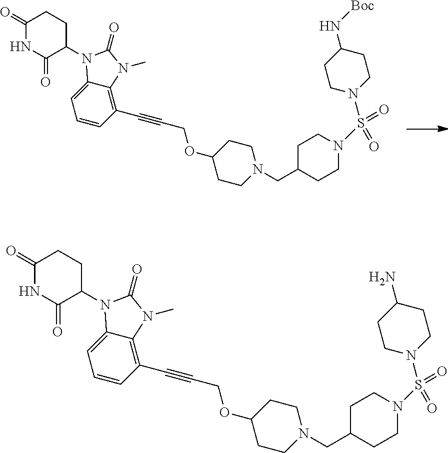 [SMILES: Cn1c(=O)n(C2CCC(=O)NC2=O)c2cccc(C#CCOC3CCN(CC4CCN(S(=O)(=O)N5CCC(NC(=O)OC(C)(C)C)CC5)CC4)CC3)c21 ; Cn1c(=O)n(C2CCC(=O)NC2=O)c2cccc(C#CCOC3CCN(CC4CCN(S(=O)(=O)N5CCC(N)CC5)CC4)CC3)c21]

Proceeding analogously as described in Reference 10, Step 7 above, but using tert-butyl (1-((4-((4-((3-(1-(2,6-dioxopiperidin-3-yl)-3-methyl-2-oxo-2,3-dihydro-1H-benzo[d]imidazol-4-yl)-prop-2-yn-1-yl)oxy)piperidin-1-yl)methyl)piperidin-1-yl)sulfonyl)piperidin-4-yl)carbamate provided the title compound.

### Reference 14

### Synthesis of 3-(4-(3-((1-((4-aminopiperidin-1-yl)sulfonyl)piperidin-4-yl)oxy)prop-1-yn-1-yl)-3-methyl-2-oxo-2,3-dihydro-1H-benzo[d]imidazol-1-yl)piperidine-2,6-dione

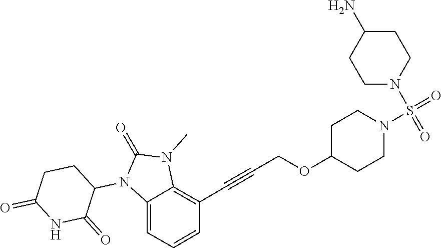 [SMILES: Cn1c(=O)n(C2CCC(=O)NC2=O)c2cccc(C#CCOC3CCN(S(=O)(=O)N4CCC(N)CC4)CC3)c21]

### Step 1: 3-Hydroxy-1-(4-methoxybenzyl)piperidine-2,6-dione

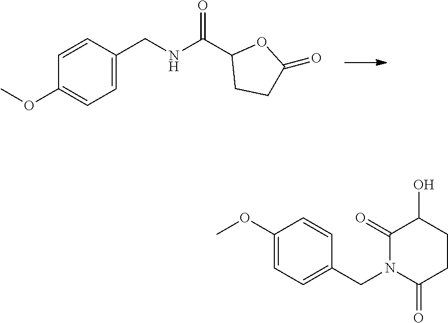 [SMILES: COc1ccc(CNC(=O)C2CCC(=O)O2)cc1 ; COc1ccc(CN2C(=O)CCC(O)C2=O)cc1]

t-BuOK (2.3 g, 20.50 mmol, 1.02 eq.) was added to a stirred mixture of N-(4-methoxybenzyl)-5-oxotetrahydrofuran-2-carboxamide (5.0 g, 20.06 mmol, 1.00 eq.) in THF (50.0 mL) at −78° C. After stirring at −78° C. for 1 h, the mixture was quenched with saturated aqueous NH₄Cl and then extracted with EtOAc. The organic layer was washed with brine, dried over Na₂SO₄, concentrated and then purified with silica gel chromatograph (PE/EA=3/1) to give the title compound as a white solid.

### Step 2: 1-(4-Methoxybenzyl)-2,6-dioxopiperidin-3-yl trifluoromethanesulfonate

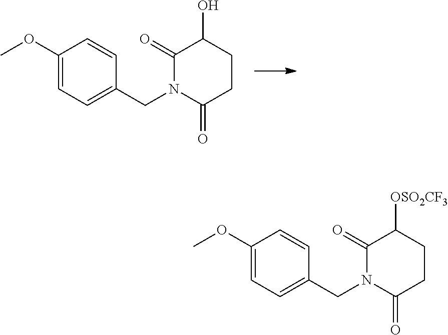

Trifluoromethanesulfonic anhydride (3.2 g, 11.34 mmol, 1.49 eq.) was added slowly to a stirred solution of 3-hydroxy-1-(4-methoxybenzyl)piperidine-2,6-dione (1.9 g, 7.62 mmol, 1.00 eq.) and pyridine (1.2 g, 15.17 mmol, 1.99 eq.) in DCM (40.0 mL) at 0° C. After stirring at 0° C. for 2 h, the reaction mixture was quenched with water and then extracted with DCM. The organic layer was washed with brine, dried over Na₂SO₄, concentrated and then purified by silica gel chromatograph (PE/EA=5/1) to give the title compound as a yellow oil.

### Step 3: 3-(4-Bromo-3-methyl-2-oxo-2,3-dihydro-1H-benzo[d]imidazol-1-yl)-1-(4-methoxy-benzyl)piperidine-2,6-dione

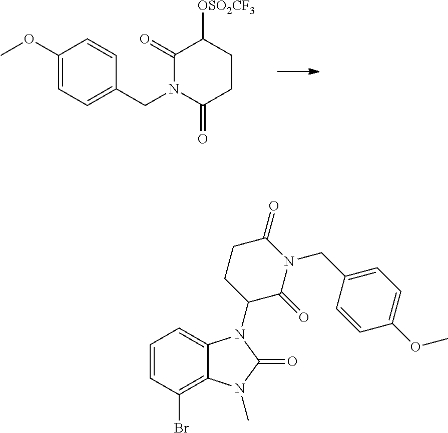

To a stirred solution of 7-bromo-1-methyl-1H-benzo[d]imidazol-2 (3H)-one (1.1 g, 4.84 mmol, 1.23 eq.) in THF (30.0 mL) was added t-BuOK (632 mg, 5.63 mmol, 1.43 eq.) at 0° C. After stirring at 0° C. for 0.5 h, a solution of 1-(4-methoxybenzyl)-2,6-dioxopiperidin-3-yl trifluoromethanesulfonate (1.5 g, 3.93 mmol, 1.00 eq.) in THF (10.0 mL) was added at 0° C. The reaction mixture was stirred at 0° C. for 1 h, diluted with water and then extracted with EtOAc. The organic layer was washed with brine, dried over Na₂SO₄, concentrated and the residue was purified by silica gel chromatograph (PE/EA=2/1) to give the title compound as a white solid.

### Step 4: 3-(4-Bromo-3-methyl-2-oxo-2,3-dihydro-1H-benzo[d]imidazol-1-yl)piperidine-2,6-dione

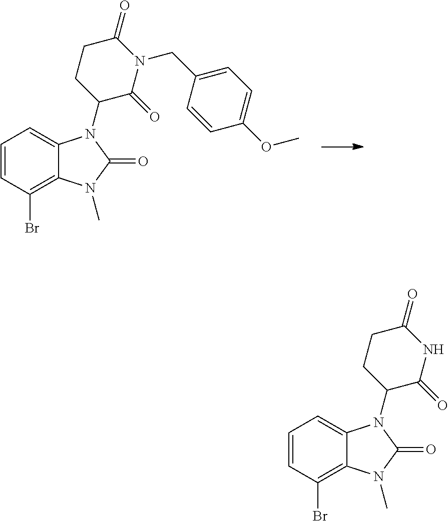 [SMILES: COc1ccc(CN2C(=O)CCC(n3c(=O)n(C)c4c(Br)cccc43)C2=O)cc1 ; Cn1c(=O)n(C2CCC(=O)NC2=O)c2cccc(Br)c21]

A mixture of 3-(4-bromo-3-methyl-2-oxo-2,3-dihydro-1H-benzo[d]imidazol-1-yl)-1-(4-methoxybenzyl)piperidine-2,6-dione (900 mg, 1.96 mmol, 1.00 eq.) in toluene/methanesulfonic acid=2/1 (3.0 mL) was stirred at 120° C. for 3 h. The reaction mixture was cooled, concentrated and poured into ice water. The resulting mixture was filtered, and the cake was dried to give the title compound as a white solid.

### Step 5: tert-Butyl 4-(prop-2-yn-1-yloxy)piperidine-1-carboxylate

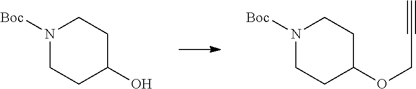 [SMILES: CC(C)(C)OC(=O)N1CCC(O)CC1 ; C#CCOC1CCN(C(=O)OC(C)(C)C)CC1]

NaH (60%, 240 mg, 6.00 mmol, 1.21 eq.) was added to a stirred solution of tert-butyl 4-hydroxypiperidine-1-carboxylate (1.0 g, 4.97 mmol, 1.00 eq.) in THF (20.0 mL) at 0° C., followed by 3-bromoprop-1-yne (704 mg, 5.92 mmol 1.19 eq.). The resulting mixture was stirred at RT for 2 h, quenched with water and then extracted with EtOAc. The organic layer was washed with brine, dried over Na₂SO₄, concentrated and then purified by silica gel chromatograph (PE/EA=10/1) to give the title compound as a white solid.

### Step 6: tert-Butyl 4-((3-(1-(2,6-dioxopiperidin-3-yl)-3-methyl-2-oxo-2,3-dihydro-1H-benzo[d]imidazol-4-yl)prop-2-yn-1-yl)oxy)piperidine-1-carboxylate

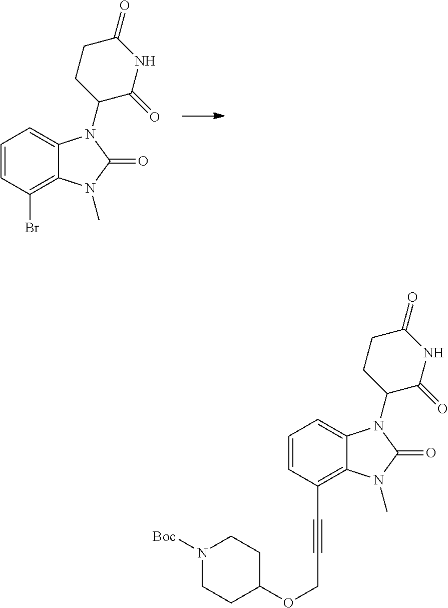 [SMILES: Cn1c(=O)n(C2CCC(=O)NC2=O)c2cccc(Br)c21 ; Cn1c(=O)n(C2CCC(=O)NC2=O)c2cccc(C#CCOC3CCN(C(=O)OC(C)(C)C)CC3)c21]

Proceeding analogously as described in Reference 10, Step 3 above, but using 3-(4-bromo-3-methyl-2-oxo-2,3-dihydro-1H-benzo[d]imidazol-1-yl)piperidine-2,6-dione and tert-butyl 4-(prop-2-yn-1-yloxy)piperidine-1-carboxylate in DMF provided the title compound.

### Step 7: 3-(3-Methyl-2-oxo-4-(3-(piperidin-4-yloxy)prop-1-yn-1-yl)-2,3-dihydro-1H-benzo[d]imidazol-1-yl)piperidine-2,6-dione

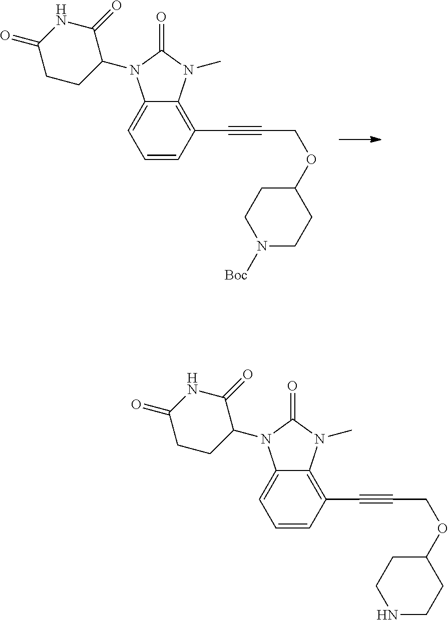 [SMILES: Cn1c(=O)n(C2CCC(=O)NC2=O)c2cccc(C#CCOC3CCN(C(=O)OC(C)(C)C)CC3)c21 ; Cn1c(=O)n(C2CCC(=O)NC2=O)c2cccc(C#CCOC3CCNCC3)c21]

Proceeding analogously as described in Reference 10, Step 5 above, but using tert-butyl 4-((3-(1-(2,6-dioxopiperidin-3-yl)-3-methyl-2-oxo-2,3-dihydro-1H-benzo[d]imidazol-4-yl)prop-2-yn-1-yl)oxy)piperidine-1-carboxylate provided the title compound.

### Step 8: tert-Butyl (1-((4-((3-(1-(2,6-dioxopiperidin-3-yl)-3-methyl-2-oxo-2,3-dihydro-1H-benzo[d]imidazol-4-yl)prop-2-yn-1-yl)oxy)piperidin-1-yl)sulfonyl)piperidin-4-yl)carbamate

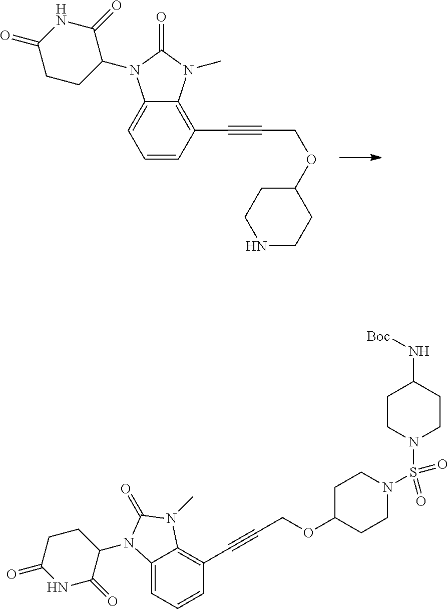 [SMILES: Cn1c(=O)n(C2CCC(=O)NC2=O)c2cccc(C#CCOC3CCNCC3)c21 ; Cn1c(=O)n(C2CCC(=O)NC2=O)c2cccc(C#CCOC3CCN(S(=O)(=O)N4CCC(NC(=O)OC(C)(C)C)CC4)CC3)c21]

Proceeding analogously as described in Reference 10, Step 6 above, but using 3-(3-methyl-2-oxo-4-(3-(piperidin-4-yloxy)prop-1-yn-1-yl)-2,3-dihydro-1H-benzo[d]imidazol-1-yl)piperidine-2,6-dione and tert-butyl (1-(chlorosulfonyl)piperidin-4-yl)carbamate in DMF provided the title compound.

### Step 9: 3-(4-(3-((1-((4-aminopiperidin-1-yl)sulfonyl)piperidin-4-yl)oxy)prop-1-yn-1-yl)-3-methyl-2-oxo-2,3-dihydro-1H-benzo[d]imidazol-1-yl)piperidine-2,6-dione

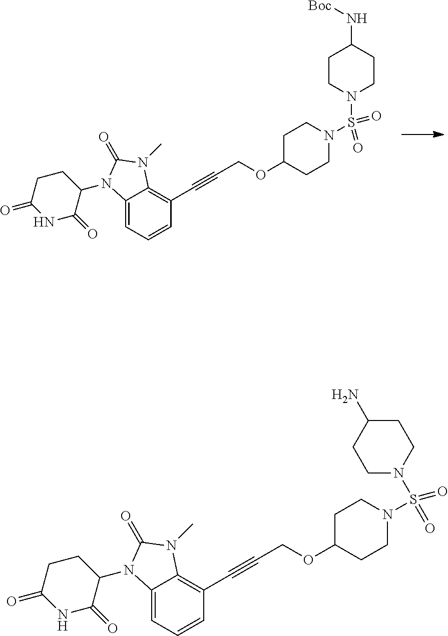 [SMILES: Cn1c(=O)n(C2CCC(=O)NC2=O)c2cccc(C#CCOC3CCN(S(=O)(=O)N4CCC(NC(=O)OC(C)(C)C)CC4)CC3)c21 ; Cn1c(=O)n(C2CCC(=O)NC2=O)c2cccc(C#CCOC3CCN(S(=O)(=O)N4CCC(N)CC4)CC3)c21]

Proceeding analogously as described in Reference 10, Step 7 above, but using tert-butyl (1-((4-((3-(1-(2,6-dioxopiperidin-3-yl)-3-methyl-2-oxo-2,3-dihydro-1H-benzo[d]imidazol-4-yl)prop-2-yn-1-yl)oxy)piperidin-1-yl)sulfonyl)piperidin-4-yl)carbamate provided the title compound.

### Reference 15

### Synthesis of 4-amino-N-(4-((2-(2,6-dioxopiperidin-3-yl)-1,3-dioxoisoindolin-4-yl)amino)-cyclohexyl)-N-methylpiperidine-1-sulfonamide 2,2,2-trifluoroacetate

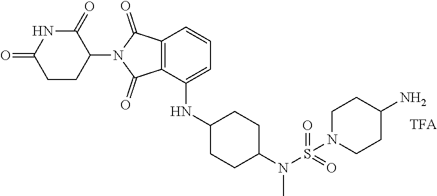 [SMILES: CN(C1CCC(Nc2cccc3c2C(=O)N(C2CCC(=O)NC2=O)C3=O)CC1)S(=O)(=O)N1CCC(N)CC1]

### Step 1: 4-Aminocyclohexanone hydrochloride

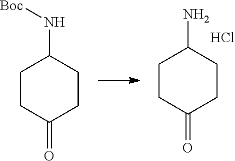 [SMILES: CC(C)(C)OC(=O)NC1CCC(=O)CC1 ; NC1CCC(=O)CC1 ; Cl]

A mixture of tert-butyl (4-oxocyclohexyl)carbamate (500 mg, 2.34 mmol, 1.00 eq.) in a solution of HCl in ethyl acetate (1.0 M, 10.0 mL) was stirred at RT for 1 h. The reaction mixture was concentrated to give the title compound, which was used for next step without further purification.

### Step 2: 2-(2,6-Dioxopiperidin-3-yl)-4-((4-oxocyclohexyl)amino) isoindoline-1,3-dione

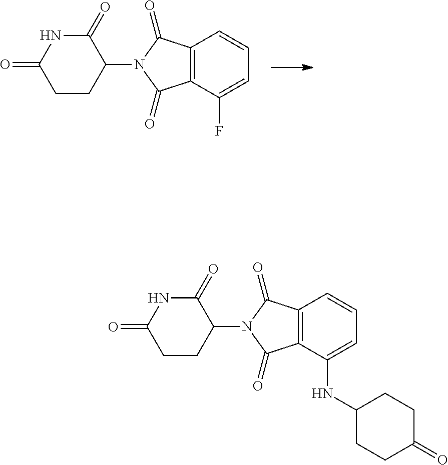 [SMILES: O=C1CCC(N2C(=O)c3cccc(F)c3C2=O)C(=O)N1 ; O=C1CCC(Nc2cccc3c2C(=O)N(C2CCC(=O)NC2=O)C3=O)CC1]

A mixture of 2-(2,6-dioxopiperidin-3-yl)-4-fluoroisoindoline-1,3-dione (276 mg, 1.00 mmol, 1.00 eq.) and 4-aminocyclohexanone hydrochloride (300 mg, 2.00 mmol, 2.00 eq.) in NMP (2.5 mL) was stirred at 140° C. under microwave for 3 h. The reaction mixture was cooled, diluted with DCM and then washed with brine. The organic layer was concentrated, and then the residue was triturated with DCM, filtered to give the title compound as a yellow solid.

### Step 3: 2-(2,6-Dioxopiperidin-3-yl)-4-((4-(methylamino)cyclohexyl)amino) isoindoline-1,3-dione

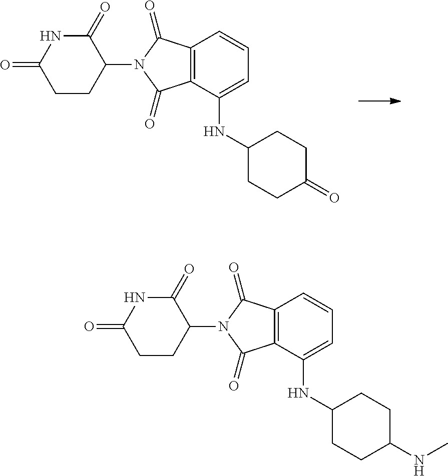 [SMILES: O=C1CCC(Nc2cccc3c2C(=O)N(C2CCC(=O)NC2=O)C3=O)CC1 ; CNC1CCC(Nc2cccc3c2C(=O)N(C2CCC(=O)NC2=O)C3=O)CC1]

To a stirred mixture of 2-(2,6-dioxopiperidin-3-yl)-4-((4-oxocyclohexyl)amino)-isoindoline-1,3-dione (200 mg, 0.54 mmol, 1.00 eq.) and methylamine (40% in MeOH, 210 mg, 2.71 mmol, 5.02 eq.) in MeOH/DCE (2.0 mL/2.0 mL) was added one drop of AcOH. The resulting mixture was stirred at RT for 1 h, and then NaBH(OAc)₃ (345 mg, 1.63 mmol, 3.02 eq.) was added. The reaction mixture was stirred at RT overnight, diluted with DCM, washed with saturated aqueous NaHCO₃ and then brine. The organic layer was dried over Na₂SO₄ and then concentrated to give the title compound as a yellow solid.

### Step 4: tert-Butyl (1-(N-(4-((2-(2,6-dioxopiperidin-3-yl)-1,3-dioxoisoindolin-4-yl)amino)-cyclohexyl)-N-methylsulfamoyl)piperidin-4-yl)carbamate

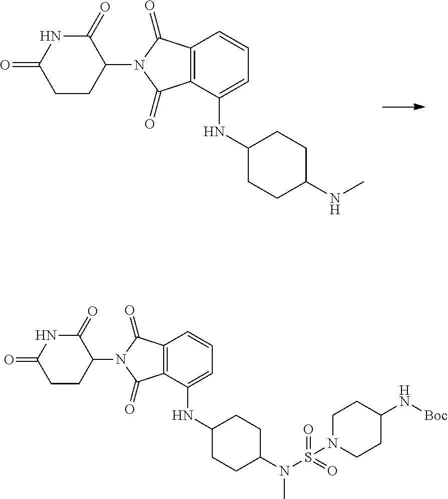 [SMILES: CNC1CCC(Nc2cccc3c2C(=O)N(C2CCC(=O)NC2=O)C3=O)CC1 ; CN(C1CCC(Nc2cccc3c2C(=O)N(C2CCC(=O)NC2=O)C3=O)CC1)S(=O)(=O)N1CCC(NC(=O)OC(C)(C)C)CC1]

Proceeding analogously as described in Reference 10, Step 6 above, but using 2-(2,6-dioxopiperidin-3-yl)-4-((4-(methylamino)cyclohexyl)amino) isoindoline-1,3-dione and tert-butyl (1-(chlorosulfonyl)piperidin-4-yl)carbamate provided the title compound.

### Step 5: 4-Amino-N-(4-((2-(2,6-dioxopiperidin-3-yl)-1,3-dioxoisoindolin-4-yl)amino)cyclohexyl)-N-methylpiperidine-1-sulfonamide 2,2,2-trifluoroacetate

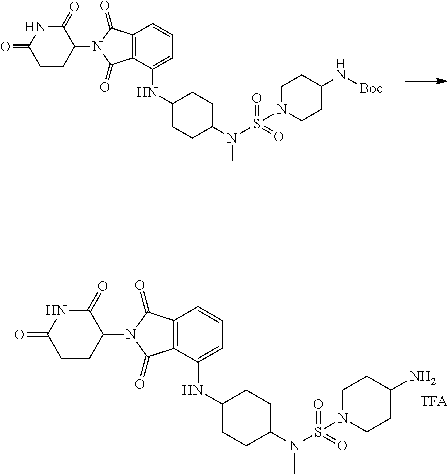 [SMILES: CN(C1CCC(Nc2cccc3c2C(=O)N(C2CCC(=O)NC2=O)C3=O)CC1)S(=O)(=O)N1CCC(NC(=O)OC(C)(C)C)CC1 ; CN(C1CCC(Nc2cccc3c2C(=O)N(C2CCC(=O)NC2=O)C3=O)CC1)S(=O)(=O)N1CCC(N)CC1]

Proceeding analogously as described in Reference 10, Step 7 above, but using tert-butyl (1-(N-(4-((2-(2,6-dioxopiperidin-3-yl)-1,3-dioxoisoindolin-4-yl)amino)cyclohexyl)-N-methylsulfamoyl)piperidin-4-yl)carbamate provided the title compound.

### Reference 16

### Synthesis of tert-butyl 4-((3-(1-(2,6-dioxopiperidin-3-yl)-3-methyl-2-oxo-2,3-dihydro-1H-benzo[d]-imidazol-4-yl)prop-2-yn-1-yl)oxy)piperidine-1-carboxylate

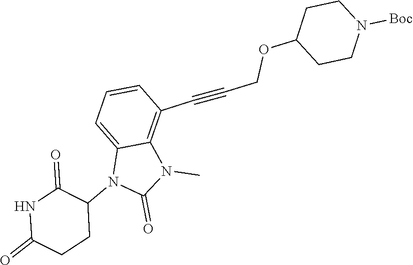 [SMILES: Cn1c(=O)n(C2CCC(=O)NC2=O)c2cccc(C#CCOC3CCN(C(=O)OC(C)(C)C)CC3)c21]

### Step 1: tert-Butyl 4-((3-(1-(2,6-dioxopiperidin-3-yl)-3-methyl-2-oxo-2,3-dihydro-1H-benzo[d]-imidazol-4-yl)prop-2-yn-1-yl)oxy)piperidine-1-carboxylate

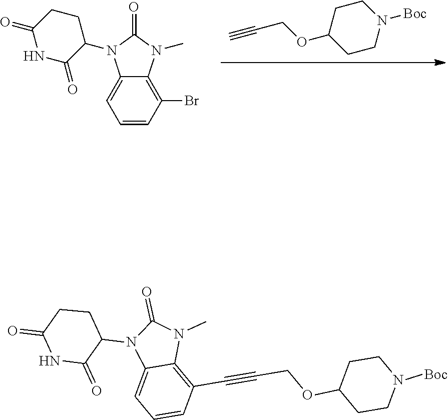 [SMILES: Cn1c(=O)n(C2CCC(=O)NC2=O)c2cccc(Br)c21 ; C#CCOC1CCN(C(=O)OC(C)(C)C)CC1 ; Cn1c(=O)n(C2CCC(=O)NC2=O)c2cccc(C#CCOC3CCN(C(=O)OC(C)(C)C)CC3)c21]

Proceeding analogously as described in Reference 10, Step 3 above, but using 3-(4-bromo-3-methyl-2-oxo-2,3-dihydro-1H-benzo[d]imidazol-1-yl)piperidine-2,6-dione and tert-butyl 4-(prop-2-yn-1-yloxy)piperidine-1-carboxylate provided the title compound.

### Reference 17

### Synthesis of 5-((3-(3-((4-aminopiperidin-1-yl)sulfonyl)phenoxy)azetidin-1-yl)methyl)-2-(2,6-dioxopiperidin-3-yl)isoindoline-1,3-dione

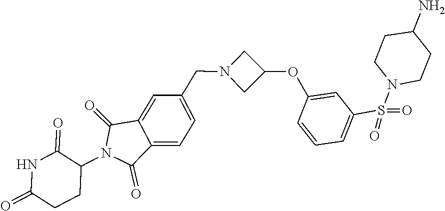 [SMILES: NC1CCN(S(=O)(=O)c2cccc(OC3CN(Cc4ccc5c(c4)C(=O)N(C4CCC(=O)NC4=O)C5=O)C3)c2)CC1]

### Step 1: tert-Butyl (1-((3-((1-((2-(2,6-dioxopiperidin-3-yl)-1,3-dioxoisoindolin-5-yl)methyl)-azetidin-3-yl)oxy)phenyl)sulfonyl)piperidin-4-yl)carbamate

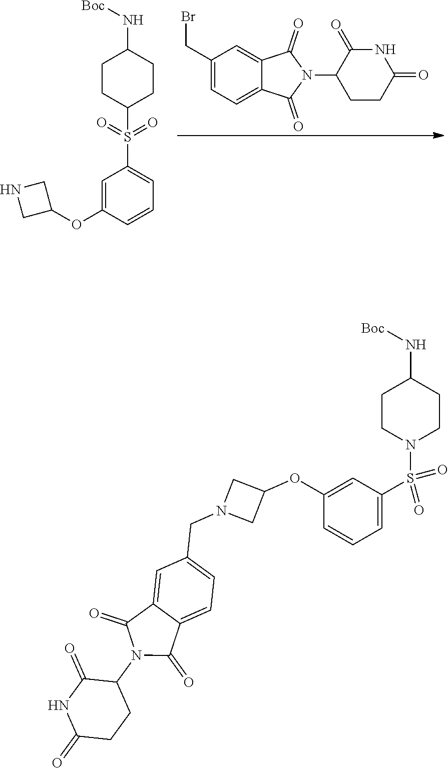

Proceeding analogously as described in Reference 8, Step 5 above, but using tert-butyl (1-((3-(azetidin-3-yloxy)phenyl)sulfonyl)piperidin-4-yl)carbamate and 5-(bromomethyl)-2-(2,6-dioxopiperidin-3-yl)isoindoline-1,3-dione provided the title compound.

### Step 2: 5-((3-(3-((4-Aminopiperidin-1-yl)sulfonyl)phenoxy)azetidin-1-yl)methyl)-2-(2,6-dioxopiperidin-3-yl)isoindoline-1,3-dione

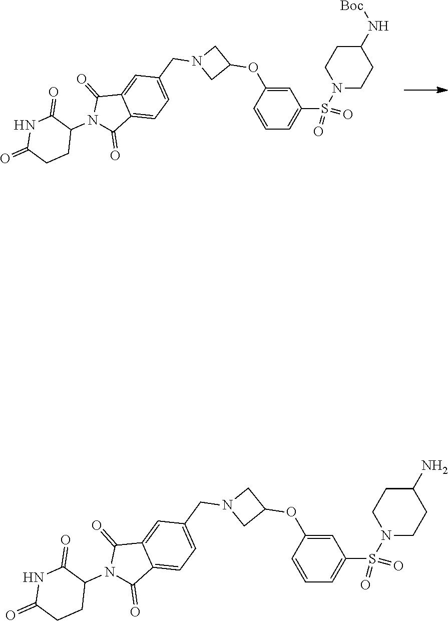 [SMILES: CC(C)(C)OC(=O)NC1CCN(S(=O)(=O)c2cccc(OC3CN(Cc4ccc5c(c4)C(=O)N(C4CCC(=O)NC4=O)C5=O)C3)c2)CC1 ; NC1CCN(S(=O)(=O)c2cccc(OC3CN(Cc4ccc5c(c4)C(=O)N(C4CCC(=O)NC4=O)C5=O)C3)c2)CC1]

Proceeding analogously as described in Reference 8, Step 6 above, but using (1-((3-((1-((2-(2,6-dioxopiperidin-3-yl)-1,3-dioxoisoindolin-5-yl)methyl)azetidin-3-yl)oxy)phenyl)-sulfonyl)piperidin-4-yl)carbamate and TFA provided the title compound.

### Reference 18

### Synthesis of 4-amino-N-(2-(3-(1-(2,6-dioxopiperidin-3-yl)-3-methyl-2-oxo-2,3-dihydro-1H-benzo[d]imidazol-4-yl)propoxy)ethyl)piperidine-1-sulfonamide

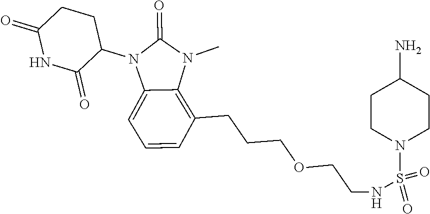 [SMILES: Cn1c(=O)n(C2CCC(=O)NC2=O)c2cccc(CCCOCCNS(=O)(=O)N3CCC(N)CC3)c21]

### Step 1: tert-Butyl(2-(prop-2-yn-1-yloxy)ethyl)carbamate

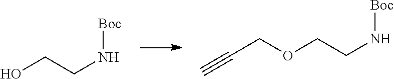

Proceeding analogously as described in Reference 10, Step 2 above, but using tert-butyl (2-hydroxyethyl)carbamate and 3-bromoprop-1-yne provided the title compound.

### Step 2: 4-Amino-N-(2-(3-(1-(2,6-dioxopiperidin-3-yl)-3-methyl-2-oxo-2,3-dihydro-1H-benzo[d]imidazol-4-yl)propoxy)ethyl)piperidine-1-sulfonamide

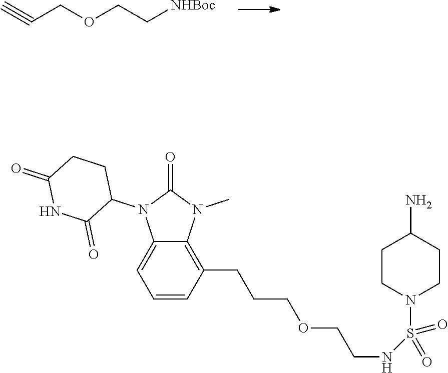 [SMILES: C#CCOCCNC(=O)OC(C)(C)C ; Cn1c(=O)n(C2CCC(=O)NC2=O)c2cccc(CCCOCCNS(=O)(=O)N3CCC(N)CC3)c21]

Proceeding analogously as described in Reference 10, Step 3-7 above, but using tert-butyl (2-(prop-2-yn-1-yloxy)ethyl)carbamate provided the title compound.

### Reference 19

### Synthesis of tert-butyl (1-((1-(2,6-dioxopiperidin-3-yl)-3-methyl-2-oxo-2,3-dihydro-1H-benzo[d]imidazol-4-yl)methyl)piperidin-4-yl)(methyl)carbamate

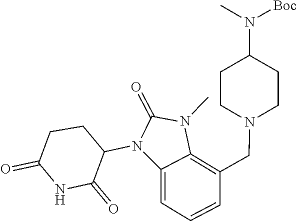 [SMILES: CN(C(=O)OC(C)(C)C)C1CCN(Cc2cccc3c2n(C)c(=O)n3C2CCC(=O)NC2=O)CC1]

### Step 1: 1-(2,6-Dioxopiperidin-3-yl)-3-methyl-2-oxo-2,3-dihydro-1H-benzo[d]imidazole-4-carbaldehyde

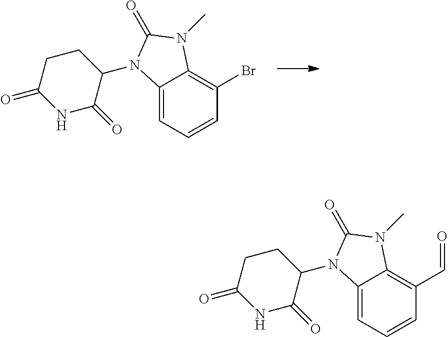 [SMILES: Cn1c(=O)n(C2CCC(=O)NC2=O)c2cccc(Br)c21 ; Cn1c(=O)n(C2CCC(=O)NC2=O)c2cccc(C=O)c21]

A mixture of 3-(4-bromo-3-methyl-2-oxo-2,3-dihydro-1H-benzo[d]imidazol-1-yl)-piperidine-2,6-dione (700 mg, 2.07 mmol, 1.00 eq.), TEA (630 mg, 6.23 mmol, 3.01 eq.), Pd(dppf)Cl₂ (230.6 mg, 0.32 mmol, 0.15 eq.), Et₃SiH (733 mg, 6.30 mmol, 3.04 eq.) in DMF (10 mL) was stirred at 80° C. under 15 psi carbon monoxide atmosphere overnight. The reaction mixture was diluted with water and then extracted with EtOAc. The organic layer was washed with brine, dried over Na₂SO₄, filtered and then concentrated. The residue was purified by chromatograph on silica gel (DCM/MeOH=20/1) to give the title compound as a yellow oil.

### Step 2: tert-Butyl (1-((1-(2,6-dioxopiperidin-3-yl)-3-methyl-2-oxo-2,3-dihydro-1H-benzo[d]imidazol-4-yl)methyl)piperidin-4-yl)(methyl)carbamate

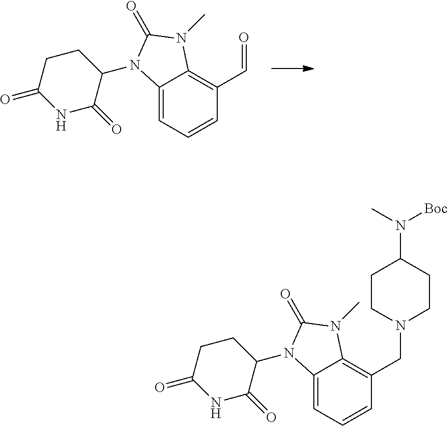 [SMILES: Cn1c(=O)n(C2CCC(=O)NC2=O)c2cccc(C=O)c21 ; CN(C(=O)OC(C)(C)C)C1CCN(Cc2cccc3c2n(C)c(=O)n3C2CCC(=O)NC2=O)CC1]

A mixture of 1-(2,6-dioxopiperidin-3-yl)-3-methyl-2-oxo-2,3-dihydro-1H-benzo[d]-imidazole-4-carbaldehyde (360 mg, 1.25 mmol, 1.00 eq.), tert-butyl N-methyl (piperidin-4-yl)carbamate (403 mg, 1.88 mmol, 1.50 eq.) in THF/DMF=2/1 (5 mL) was stirred at RT for 2 h. NaBH(OAC)₃ (413 mg, 1.95 mmol, 1.60 eq.) was added at RT. After the reaction was complete, the mixture was diluted with water and then extracted with EtOAc. The organic layer was washed with brine, dried over Na₂SO₄ and then concentrated. The residue was purified by Prep-HPLC to give the title compound as a yellow solid.

### Reference 20

### Synthesis of tert-butyl(2-((4-((2-(2,6-dioxopiperidin-3-yl)-1,3-dioxoisoindolin-4-yl)(methyl)amino)benzyl)(methyl)amino)ethyl)(methyl)carbamate

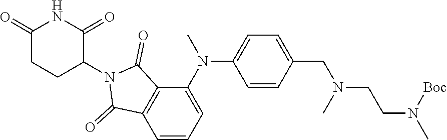 [SMILES: CN(CCN(C)C(=O)OC(C)(C)C)Cc1ccc(N(C)c2cccc3c2C(=O)N(C2CCC(=O)NC2=O)C3=O)cc1]

### Step 1: Dimethyl 3-iodophthalate

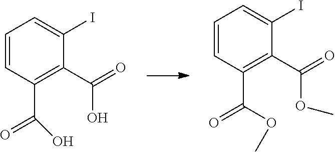

To a stirred mixture of 3-iodophthalic acid (5.00 g, 17.12 mmol, 1.00 eq.), Na₂CO₃ (5.40 g, 50.95 mmol, 2.98 eq.) in DMF (30 mL) was added iodomethane (7.30 g, 51.43 mmol, 3.00 eq.) at RT. The reaction mixture was stirred at 70° C. overnight, cooled, diluted with water, and extracted with EtOAc. The organic layer was washed with brine, dried over Na₂SO₄, filtered, and concentrated. The residue was purified by chromatograph on silica gel (PE/EA=10/1) to give the title compound as a white solid.

### Step 2: 4-(((tert-Butyldimethylsilyl)oxy)methyl)aniline

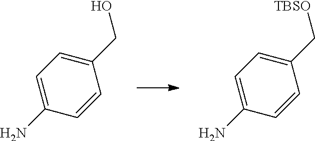 [SMILES: Nc1ccc(CO)cc1 ; CC(C)(C)[Si](C)(C)OCc1ccc(N)cc1]

A mixture of (4-aminophenyl) methanol (2.00 g, 16.24 mmol, 1.00 eq.), DMAP (595 mg, 4.87 mmol, 0.30 eq.), TEA (2.00 g, 19.76 mmol, 1.22 eq.) and TBSCl (2.70 g, 17.91 mmol, 1.10 eq.) in DMF (40 mL) was stirred at RT overnight. The reaction mixture was diluted with water and then extracted with EtOAc. The organic layer was washed with brine, dried over Na₂SO₄, filtered, and then concentrated. The residue was purified by chromatograph on silica gel (PE/EA=10/1) to give the title compound as a colorless oil.

### Step 3: Dimethyl 3-((4-(((tert-butyldimethylsilyl)oxy)methyl)phenyl)amino)phthalate

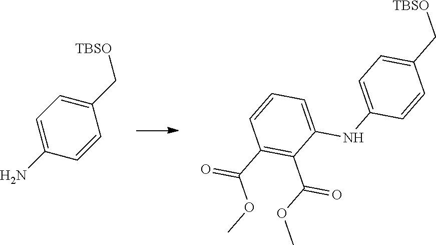 [SMILES: CC(C)(C)[Si](C)(C)OCc1ccc(N)cc1 ; COC(=O)c1cccc(Nc2ccc(CO[Si](C)(C)C(C)(C)C)cc2)c1C(=O)OC]

A mixture of 3-iodo-phthalic acid dimethyl ester (3.00 g, 9.37 mmol, 1.00 eq.), 4-(tert-butyl-dimethyl-silanyloxymethyl)-phenylamine (2.67 g, 11.25 mmol, 1.20 eq.), Pd₂(dba)₃ (436 mg, 0.48 mmol, 0.051 eq.), Cs₂CO₃ (6.11 g, 18.75 mmol, 2.00 eq.), BINAP (143 mg, 0.23 mmol, 0.025 eq.) in toluene (30.0 mL) was stirred at 120° C. overnight under nitrogen atmosphere. The reaction mixture was cooled, concentrated and the residue was purified by chromatograph on silica gel (PE/EA=10/1) to give the title compound as a yellow oil.

### Step 4: Dimethyl 3-((4-(((tert-butyldimethylsilyl)oxy)methyl)phenyl)(methyl)amino)phthalate

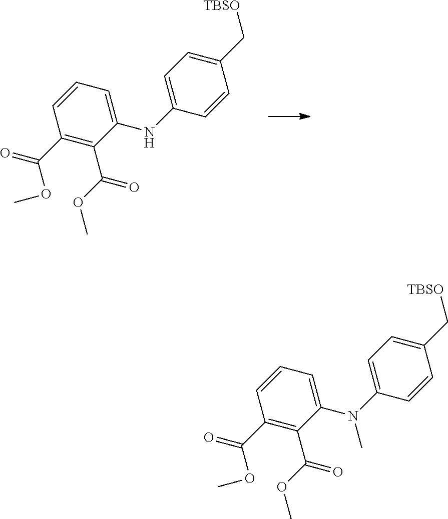 [SMILES: COC(=O)c1cccc(Nc2ccc(CO[Si](C)(C)C(C)(C)C)cc2)c1C(=O)OC ; COC(=O)c1cccc(N(C)c2ccc(CO[Si](C)(C)C(C)(C)C)cc2)c1C(=O)OC]

A mixture of dimethyl 3-((4-(((tert-butyl dimethylsilyl)oxy)methyl)phenyl)-amino) phthalate (1.50 g, 3.49 mmol, 1.00 eq.), iodomethane (991 mg, 6.98 mmol, 2.00 eq.), Cs₂CO₃ (3.41 g, 10.47 mmol, 3.00 eq.) in DMF (30.0 mL) was stirred at 20° C. for 8 h under nitrogen atmosphere. The reaction mixture was diluted with water and extracted with EtOAc. The organic layer was washed with brine, dried over Na₂SO₄, filtered, and then concentrated. The residue was purified by chromatograph on silica gel (PE/EA=5/1) to give the title compound as a yellow oil.

### Step 5: 3-[(4-Hydroxymethyl-phenyl)-methyl-amino]-phthalic acid dimethyl ester

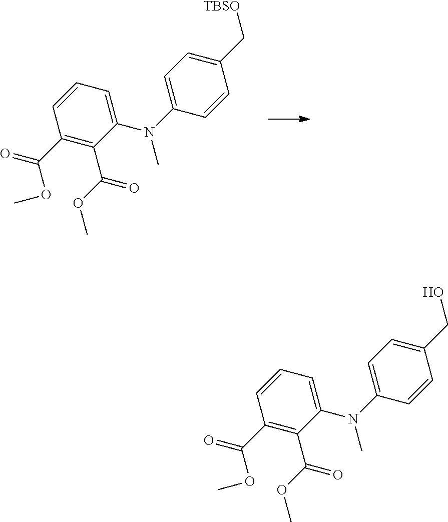 [SMILES: COC(=O)c1cccc(N(C)c2ccc(CO[Si](C)(C)C(C)(C)C)cc2)c1C(=O)OC ; COC(=O)c1cccc(N(C)c2ccc(CO)cc2)c1C(=O)OC]

A solution of TBAF in THF (3.0 M, 2.0 mL) was added To a stirred solution of dimethyl 3-((4-(((tert-butyl dimethylsilyl)oxy)methyl)phenyl)-(methyl)amino)phthalate (500 mg, 1.13 mmol, 1.00 eq.) in THF (5.0 mL) at rt. After 2 h, the mixture was diluted with water and then extracted with EtOAc. The organic layer was washed with brine, dried over Na₂SO₄, filtered and then concentrated. The residue was purified by chromatograph on silica gel (PE/EA=2/1) to give the title compound as a yellow oil.

### Step 6: Dimethyl 3-((4-formylphenyl)(methyl)amino)phthalate

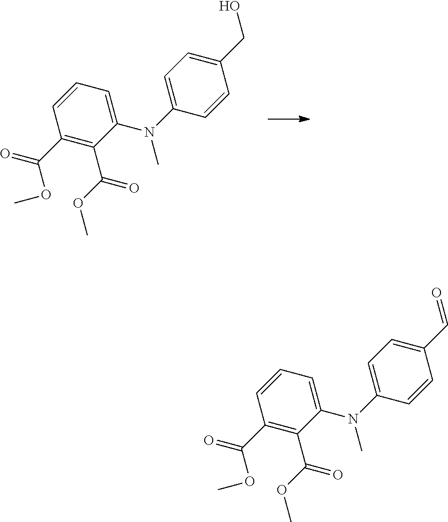 [SMILES: COC(=O)c1cccc(N(C)c2ccc(CO)cc2)c1C(=O)OC ; COC(=O)c1cccc(N(C)c2ccc(C=O)cc2)c1C(=O)OC]

A mixture of 3-[(4-hydroxymethyl-phenyl)methylamino]phthalic acid dimethyl ester (300 mg, 0.91 mmol, 1.00 eq.) and MnO₂ (800 mg, 9.20 mmol, 10.11 eq.) in DCM (10.0 mL) was stirred at rt overnight. The reaction mixture was filtered and concentrated to give the title compound as a yellow oil, which was used for next step without further purification.

### Step 7: Dimethyl 3-((4-(((2-((tert-butoxycarbonyl)(methyl)amino)ethyl)(methyl)amino)methyl) phenyl)(methyl)amino)phthalate

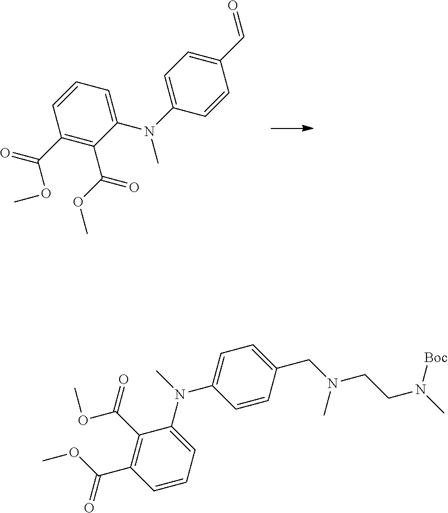

A mixture of dimethyl 3-((4-formylphenyl)(methyl)amino)phthalate (300 mg, 0.92 mmol, 1.00 eq.), methyl-(2-methylamino-ethyl)-carbamic acid tert-butyl ester (205 mg, 1.09 mmol, 1.18 eq.) and a drop of AcOH in DCE (5.0 mL) was stirred at RT for 2 h. NaBH(OAc)₃ (290 mg, 1.37 mmol, 1.49 eq.) was then added and stirred for 4 h. The reaction mixture was concentrated and purified by prep-HPLC to give the title compound as a white solid.

### Step 8: 3-((4-(((2-((tert-Butoxycarbonyl)(methyl)amino)ethyl)(methyl)amino)methyl)phenyl) (methyl)amino)phthalic acid

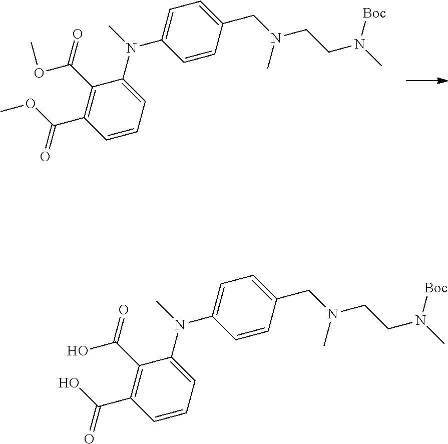

A mixture of dimethyl 3-((4-(((2-((tert-butoxycarbonyl)(methyl)amino)ethyl)(methyl)amino)methyl)phenyl)(methyl)amino)phthalate (250 mg, 0.50 mmol, 1.00 eq.) and NaOH (40 mg, 1.00 mmol, 2.00 eq.) in EtOH/H₂O=2/1 (5.0 mL) was stirred at 80° C. for 5 h. The reaction mixture was concentrated and purified by prep-HPLC to give the title compound as a white solid.

### Step 9: tert-Butyl(2-((4-((2-(2,6-dioxopiperidin-3-yl)-1,3-dioxoisoindolin-4-yl)(methyl)amino) benzyl)(methyl)amino)ethyl)(methyl)carbamate

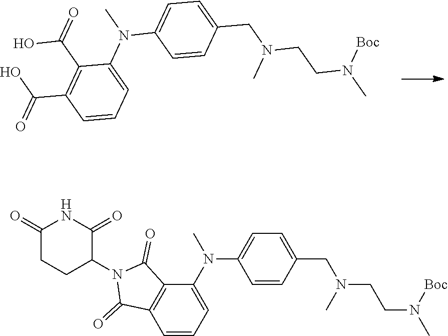 [SMILES: CN(CCN(C)C(=O)OC(C)(C)C)Cc1ccc(N(C)c2cccc(C(=O)O)c2C(=O)O)cc1 ; CN(CCN(C)C(=O)OC(C)(C)C)Cc1ccc(N(C)c2cccc3c2C(=O)N(C2CCC(=O)NC2=O)C3=O)cc1]

A mixture of 3-((4-(((2-((tert-butoxycarbonyl)(methyl)amino)ethyl)(methyl)amino)methyl) phenyl)(methyl)amino)phthalic acid (120 mg, 0.25 mmol, 1.00 eq.) and 3-aminopiperidine-2,6-dione hydrochloride (41 mg, 0.25 mmol, 1.00 eq.) in pyridine (3.0 mL) was stirred at 100° C. overnight. The reaction mixture was cooled and concentrated. The residue was purified by chromatograph on silica gel (DCM/MeOH=30/1) to give the title compound as a yellow solid.

### Reference 21

### Synthesis of tert-butyl (3-(4-(1-(2,6-dioxopiperidin-3-yl)-3-methyl-2-oxo-2,3-dihydro-1H-benzo[d]-imidazol-4-yl)piperidin-1-yl)propyl)(methyl)carbamate

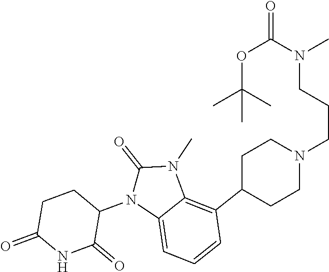 [SMILES: CN(CCCN1CCC(c2cccc3c2n(C)c(=O)n3C2CCC(=O)NC2=O)CC1)C(=O)OC(C)(C)C]

### Step 1: tert-Butyl 4-(1-(2,6-dioxopiperidin-3-yl)-3-methyl-2-oxo-2,3-dihydro-1H-benzo[d]-imidazol-4-yl)-3,6-dihydropyridine-1(2H)-carboxylate

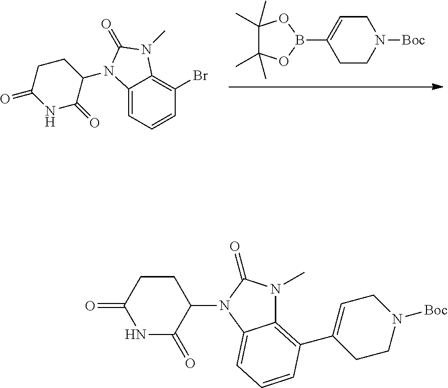 [SMILES: Cn1c(=O)n(C2CCC(=O)NC2=O)c2cccc(Br)c21 ; CC(C)(C)OC(=O)N1CC=C(B2OC(C)(C)C(C)(C)O2)CC1 ; Cn1c(=O)n(C2CCC(=O)NC2=O)c2cccc(C3=CCN(C(=O)OC(C)(C)C)CC3)c21]

A mixture of 3-(4-bromo-3-methyl-2-oxo-2,3-dihydro-1H-benzo[d]imidazol-1-yl)-piperidine-2,6-dione (100 mg, 0.30 mmol, 1.00 eq.), tert-butyl 4-(4,4,5,5-tetramethyl-1,3,2-dioxaborolan-2-yl)-5,6-dihydropyridine-1(2H)-carboxylate (119 mg, 0.38 mmol, 1.27 eq.), X-Phos-Pd-G3 (38 mg, 0.045 mmol, 0.15 eq.), and K₃PO₄ (191 mg, 0.90 mmol, 3.0 eq.) in 1,4-dioxane/H₂O=10/1 (2.2 mL) was stirred at 60° C. for 3 h. The reaction mixture was diluted with water and then extracted with EtOAc. The organic layer was washed with brine, dried over Na₂SO₄, filtered, and then concentrated. The residue was purified by chromatograph on silica gel (DCM/MeOH=20/1) to give the title compound as a brown solid.

### Step 2: tert-Butyl 4-(1-(2,6-dioxopiperidin-3-yl)-3-methyl-2-oxo-2,3-dihydro-1H-benzo[d]imidazol-4-yl)piperidine-1-carboxylate

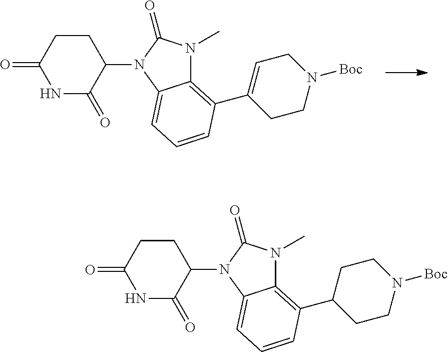 [SMILES: Cn1c(=O)n(C2CCC(=O)NC2=O)c2cccc(C3=CCN(C(=O)OC(C)(C)C)CC3)c21 ; Cn1c(=O)n(C2CCC(=O)NC2=O)c2cccc(C3CCN(C(=O)OC(C)(C)C)CC3)c21]

A mixture of tert-butyl 4-(1-(2,6-dioxopiperidin-3-yl)-3-methyl-2-oxo-2,3-dihydro-1H-benzo[d]imidazol-4-yl)-5,6-dihydropyridine-1(2H)-carboxylate (70 mg, 0.16 mmol, 1.00 eq.), 10% Pd/C (30 mg) and Pd(OH)₂ (30 mg) in THF (10 mL) was stirred at 50° C. under 50 psi H₂ pressure. The reaction mixture was filtered and then concentrated to give the title as a white solid.

### Step 3: 3-(3-Methyl-2-oxo-4-(piperidin-4-yl)-2,3-dihydro-1H-benzo[d]imidazol-1-yl)piperidine-2,6-dione TFA salt

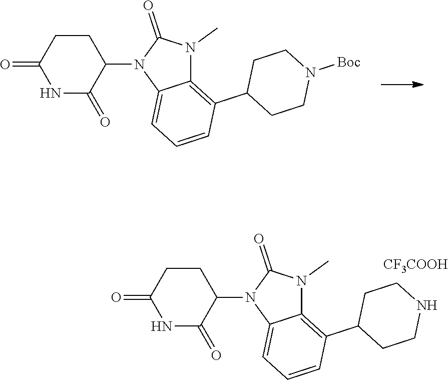 [SMILES: Cn1c(=O)n(C2CCC(=O)NC2=O)c2cccc(C3CCN(C(=O)OC(C)(C)C)CC3)c21 ; Cn1c(=O)n(C2CCC(=O)NC2=O)c2cccc(C3CCNCC3)c21 ; "CF3COOH"]

A mixture of tert-butyl 4-(1-(2,6-dioxopiperidin-3-yl)-3-methyl-2-oxo-2,3-dihydro-1H-benzo[d]imidazol-4-yl)piperidine-1-carboxylate (60 mg, 0.14 mmol, 1.00 eq.) and TFA (0.5 mL) in DCM (2 mL) was stirred at RT for 2 h. The reaction mixture was concentrated to give the title compound as a yellow oil.

### Step 4: tert-Butyl (3-(4-(1-(2,6-dioxopiperidin-3-yl)-3-methyl-2-oxo-2,3-dihydro-1H-benzo[d]-imidazol-4-yl)piperidin-1-yl)propyl)(methyl)carbamate

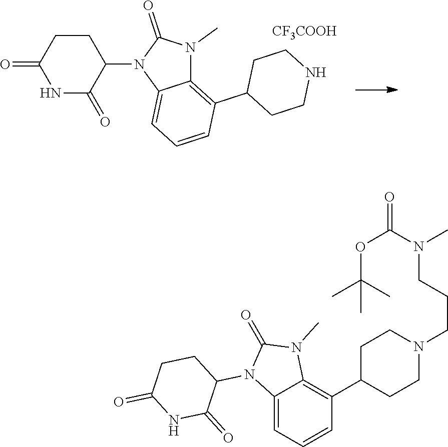 [SMILES: Cn1c(=O)n(C2CCC(=O)NC2=O)c2cccc(C3CCNCC3)c21 ; "CF3COOH" ; CN(CCCN1CCC(c2cccc3c2n(C)c(=O)n3C2CCC(=O)NC2=O)CC1)C(=O)OC(C)(C)C]

To a stirred mixture of 3-(3-methyl-2-oxo-4-(piperidin-4-yl)-2,3-dihydro-1H-benzo[d]imidazol-1-yl)piperidine-2,6-dione TFA salt (60 mg, 0.13 mmol, 1.00 eq.) in THF (5.0 mL) and DMF (1.0 mL) was added one drop of AcOH. After stirring at RT for 0.5 h, tert-butyl methyl (3-oxopropyl)carbamate (63.6 mg, 0.34 mmol, 2.0 eq) was added at RT. The mixture was stirred at 20° C. for 2 h followed by addition of NaBH(OAC)₃ (72 mg, 0.34 mmol, 2.62 eq.). After stirring at RT overnight, the mixture was diluted with water and then extracted with EtOAc. The organic layer was washed with brine, dried over Na₂SO₄, filtered, and then concentrated. The residue was purified by chromatograph on silica gel (DCM/MeOH=50/1) to give the title compound as a yellow solid.

### Reference 22

### Synthesis of tert-butyl (14-((2-(2,6-dioxopiperidin-3-yl)-1,3-dioxoisoindolin-4-yl)amino)-14-oxo-3,6,9,12-tetraoxatetradecyl)carbamate

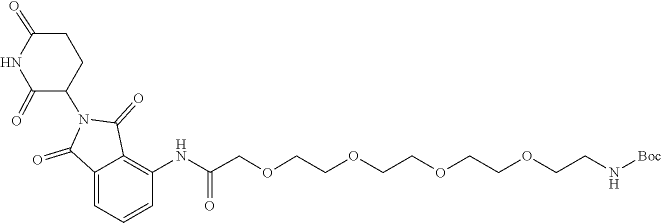 [SMILES: CC(C)(C)OC(=O)NCCOCCOCCOCCOCC(=O)Nc1cccc2c1C(=O)N(C1CCC(=O)NC1=O)C2=O]

### Step 1: 2,2-Dimethyl-4-oxo-3,8,11,14,17-pentaoxa-5-azanonadecan-19-oic acid

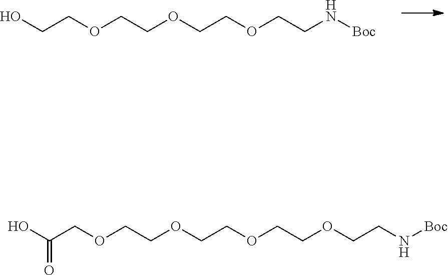 [SMILES: CC(C)(C)OC(=O)NCCOCCOCCOCCO ; CC(C)(C)OC(=O)NCCOCCOCCOCCOCC(=O)O]

NaH (60% in mineral oil, 204 mg, 5.10 mmol, 3.00 eq.) was added to a stirred solution of tert-butyl(2-(2-(2-(2-hydroxyethoxy)ethoxy)ethoxy)ethyl)carbamate (500 mg, 1.70 mmol, 1.00 eq.) in DMF (2 mL) at 0° C. under nitrogen. After stirring at 0° C. for 1 h, 2-iodoacetic acid (793 mg, 4.26 mmol, 2.51 eq.) was added at 0° C. The resulting mixture was slowly warmed to RT and then stirred overnight. This reaction mixture was quenched with H₂O at 0° C., the pH was adjusted to 2˜3 with 1 N aqueous HCl and then extracted with EtOAc. The organic layer was washed with brine, dried over Na₂SO₄, filtered, and then concentrated to give the title compound as a yellow oil.

### Step 2: tert-Butyl (14-((2-(2,6-dioxopiperidin-3-yl)-1,3-dioxoisoindolin-4-yl)amino)-14-oxo-3,6,9,12-tetraoxatetradecyl)carbamate

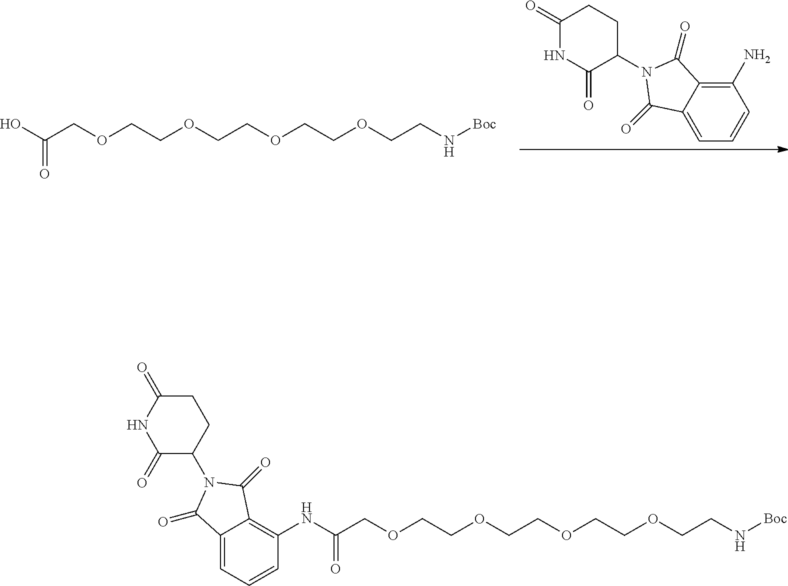 [SMILES: CC(C)(C)OC(=O)NCCOCCOCCOCCOCC(=O)O ; Nc1cccc2c1C(=O)N(C1CCC(=O)NC1=O)C2=O ; CC(C)(C)OC(=O)NCCOCCOCCOCCOCC(=O)Nc1cccc2c1C(=O)N(C1CCC(=O)NC1=O)C2=O]

To a stirred solution of 2,2-dimethyl-4-oxo-3,8,11,14,17-pentaoxa-5-azanonadecan-19-oic acid (372 mg, 1.06 mmol, 2.00 eq.) in THF (6 mL) was added isobutyl chloroformate (109 mg, 0.80 mmol, 1.51 eq.) and N-methylmorpholine (161 mg, 1.59 mmol, 3.00 eq.), followed by a solution of 4-amino-2-(2,6-dioxopiperidin-3-yl)isoindoline-1,3-dione (145 mg, 0.53 mmol, 1.00 eq.) in DMF (2 mL) dropwise at 0° C. The resulting mixture was stirred at 30° C. overnight, quenched with saturated NaHCO₃, extracted with EtOAc. The organic layer was washed with brine, dried over Na₂SO₄, filtered and then concentrated. The residue was purified by column chromatography on silica gel (PE:EA=1:1) to give the title compound as a yellow solid.

### Reference 23

### Synthesis of tert-butyl (1-((3-((1-(1-(2-(2,6-dioxopiperidin-3-yl)-1-oxoisoindolin-5-yl)piperidin-4-yl)azetidin-3-yl)oxy)phenyl)sulfonyl)piperidin-4-yl)carbamate

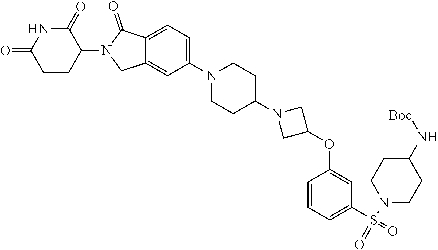 [SMILES: CC(C)(C)OC(=O)NC1CCN(S(=O)(=O)c2cccc(OC3CN(C4CCN(c5ccc6c(c5)CN(C5CCC(=O)NC5=O)C6=O)CC4)C3)c2)CC1]

### Step 1: Benzyl 4-(3-(3-((4-((tert-butoxycarbonyl)amino)piperidin-1-yl)sulfonyl) phenoxy)-azetidin-1-yl)piperidine-1-carboxylate

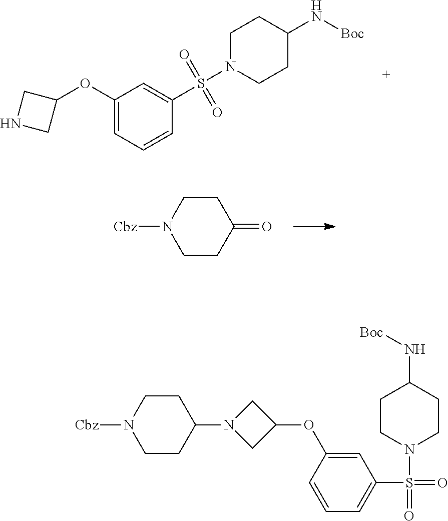 [SMILES: CC(C)(C)OC(=O)NC1CCN(S(=O)(=O)c2cccc(OC3CNC3)c2)CC1 ; O=C1CCN(C(=O)OCc2ccccc2)CC1 ; CC(C)(C)OC(=O)NC1CCN(S(=O)(=O)c2cccc(OC3CN(C4CCN(C(=O)OCc5ccccc5)CC4)C3)c2)CC1]

A solution of tert-butyl (1-((3-(azetidin-3-yloxy)phenyl)sulfonyl)piperidin-4-yl)carbamate (100 mg, 0.24 mmol, 1.00 eq.), benzyl 4-oxopiperidine-1-carboxylate (113 mg, 0.48 mmol, 2.00 eq.) and 1 drop of AcOH in THF (3.0 mL) was stirred at RT for 1 h, followed by addition of NaBH(OAc)₃ (102 mg, 0.48 mmol, 2.00 eq.). The reaction mixture was stirred at RT overnight, diluted with water and then extracted with DCM. The organic layer was concentrated and then purified by silica gel flash column (DCM/MeOH=20/1) to give the title compound as a white solid.

### Step 2: tert-Butyl (1-((3-((1-(piperidin-4-yl)azetidin-3-yl)oxy)phenyl)sulfonyl)piperidin-4-yl)-carbamate

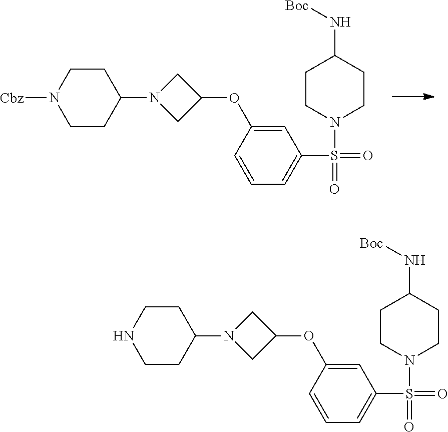 [SMILES: CC(C)(C)OC(=O)NC1CCN(S(=O)(=O)c2cccc(OC3CN(C4CCN(C(=O)OCc5ccccc5)CC4)C3)c2)CC1 ; CC(C)(C)OC(=O)NC1CCN(S(=O)(=O)c2cccc(OC3CN(C4CCNCC4)C3)c2)CC1]

To a stirred solution of benzyl 4-(3-(3-((4-((tert-butoxycarbonyl)amino)piperidin-1-yl)sulfonyl) phenoxy) azetidin-1-yl)piperidine-1-carboxylate (60 mg, 0.095 mmol, 1.00 eq.) in MeOH (10.0 mL) was added 10% Pd/C (20 mg). The resulting mixture was stirred at 45° C. under H₂ atmosphere overnight. The reaction mixture was filtered and concentrated to give the title compound as a white solid.

### Step 3: tert-Butyl (1-((3-((1-(1-(2-(2,6-dioxopiperidin-3-yl)-1-oxoisoindolin-5-yl)piperidin-4-yl)azetidin-3-yl)oxy)phenyl)sulfonyl)piperidin-4-yl)carbamate

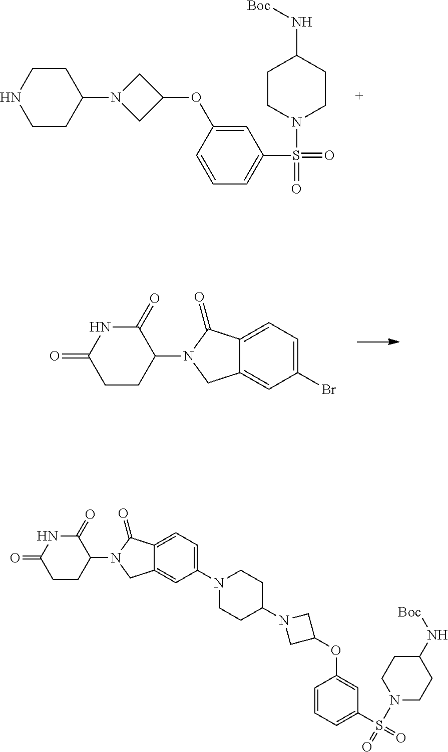 [SMILES: CC(C)(C)OC(=O)NC1CCN(S(=O)(=O)c2cccc(OC3CN(C4CCNCC4)C3)c2)CC1 ; O=C1CCC(N2Cc3cc(Br)ccc3C2=O)C(=O)N1 ; CC(C)(C)OC(=O)NC1CCN(S(=O)(=O)c2cccc(OC3CN(C4CCN(c5ccc6c(c5)CN(C5CCC(=O)NC5=O)C6=O)CC4)C3)c2)CC1]

A mixture of tert-butyl (1-((3-((1-(piperidin-4-yl)-276-zetidine-3-yl)oxy)phenyl) sulfonyl)piperidin-4-yl)carbamate (39.6 mg, 0.080 mmol, 1.00 eq.), 3-(5-bromo-1-oxoisoindolin-2-yl)piperidine-2,6-dione (38 mg, 0.12 mmol, 1.50 eq.), Cs₂CO₃ (78 mg, 0.24 mmol, 3.00 eq.), Xantphos (15 mg, 0.027 mmol, 0.34 eq.) and Pd(OAc)₂ (15 mg, 0.067 mmol, 0.84 eq.) in 1,4-dioxane (2.0 mL) was stirred at 100° C. overnight under N₂ atmosphere. The mixture was cooled and then filtered. The filtrate was diluted with water and then extracted with DCM. The organic layer was concentrated and then purified by prep-TLC (DCM/MeOH=10/1) to give the title compound as a yellow solid.

### Reference 24

### Synthesis of tert-butyl (1-((3-((1-(azetidin-3-yl)piperidin-4-yl)oxy)phenyl)sulfonyl)piperidin-4-yl)-carbamate

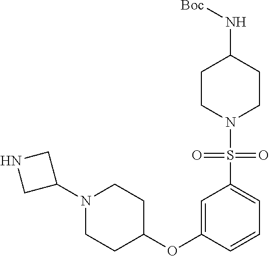 [SMILES: CC(C)(C)OC(=O)NC1CCN(S(=O)(=O)c2cccc(OC3CCN(C4CNC4)CC3)c2)CC1]

### Step 1: tert-Butyl (1-((3-(piperidin-4-yloxy)phenyl)sulfonyl)piperidin-4-yl)carbamate

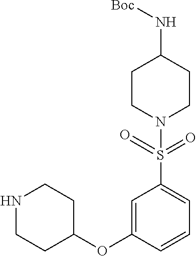 [SMILES: CC(C)(C)OC(=O)NC1CCN(S(=O)(=O)c2cccc(OC3CCNCC3)c2)CC1]

The title compound was prepared by proceeding as described in Reference 12, Steps 1 to 6 using 1-benzhydrylpiperidin-4-yl methanesulfonate.

### Step 2: tert-Butyl (1-((3-((1-(azetidin-3-yl)piperidin-4-yl)oxy)phenyl)sulfonyl)piperidin-4-yl)-carbamate

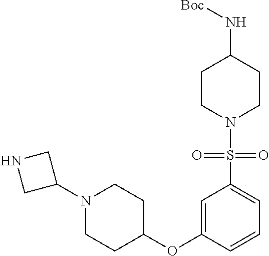 [SMILES: CC(C)(C)OC(=O)NC1CCN(S(=O)(=O)c2cccc(OC3CCN(C4CNC4)CC3)c2)CC1]

tert-Butyl (1-((3-(piperidin-4-yloxy)phenyl)sulfonyl)piperidin-4-yl)carbamate was converted to the title compound by proceeding analogously as described in Reference 27, Steps 1 and 2 above using benzyl 3-oxoazetidine-1-carboxylate.

### Reference 25

### Synthesis of tert-butyl (1-((3-((1-(2-((2-(2,6-dioxopiperidin-3-yl)-1-oxoisoindolin-4-yl)-oxy)ethyl)piperidin-4-yl)oxy)phenyl)sulfonyl)piperidin-4-yl)carbamate

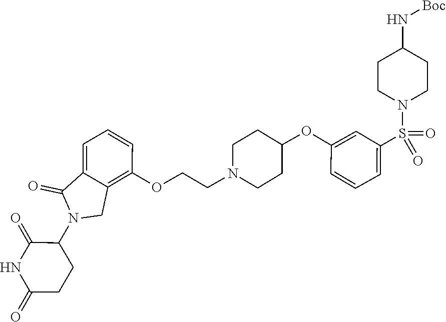 [SMILES: CC(C)(C)OC(=O)NC1CCN(S(=O)(=O)c2cccc(OC3CCN(CCOc4cccc5c4CN(C4CCC(=O)NC4=O)C5=O)CC3)c2)CC1]

### Step 1: Methyl 3-(2-hydroxyethoxy)-2-methylbenzoate

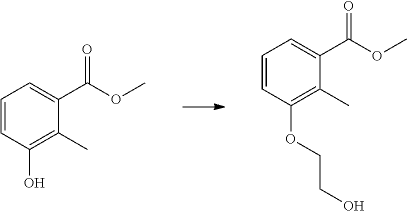

A mixture of methyl 3-hydroxy-2-methylbenzoate (2.50 g, 15.04 mmol, 1.00 eq.) and 1,3-dioxolan-2-one (1.98 g, 22.48 mmol, 1.50 eq.), K₂CO₃ (2.07 g, 14.98 mmol, 1.00 eq.) in DMF (30.0 mL) was stirred at 120° C. under N₂ for 2 h. The reaction mixture was cooled, diluted with water and then extracted with EtOAc. The organic layer was washed water, brine, dried over Na₂SO₄, filtered, and then concentrated. The residue was purified by silica gel chromatography (EA:PE=1:4) to give the title compound as a white solid.

### Step 2: Methyl 2-(bromomethyl)-3-(2-hydroxyethoxy)benzoate

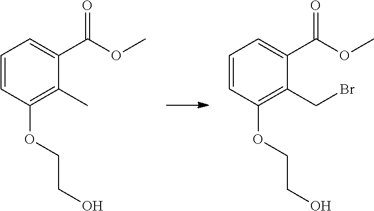 [SMILES: COC(=O)c1cccc(OCCO)c1C ; COC(=O)c1cccc(OCCO)c1CBr]

A mixture of methyl 3-(2-hydroxyethoxy)-2-methylbenzoate (1.50 g, 7.14 mmol, 1.00 eq.) in CC14 (45.0 mL), NBS (1.46 g, 8.20 mmol, 1.15 eq.) and AIBN (117 mg, 0.71 mmol, 0.10 eq was stirred under N₂ at 75° C. for 3 h. The mixture was cooled and then concentrated. The residue was purified by silica gel chromatography (EA PE=1:3) to give the title compound as a white solid.

### Step 3: 3-(4-(2-Hydroxyethoxy)-1-oxoisoindolin-2-yl)piperidine-2,6-dione

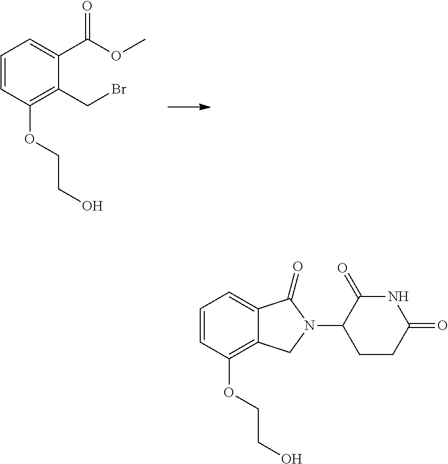 [SMILES: COC(=O)c1cccc(OCCO)c1CBr ; O=C1CCC(N2Cc3c(OCCO)cccc3C2=O)C(=O)N1]

To a stirred solution of methyl 2-(bromomethyl)-3-(2-hydroxyethoxy)benzoate (2.00 g, 6.92 mmol, 1.00 eq.) in ACN (70.0 mL) was added 3-aminopiperidine-2,6-dione hydrochloride (1.48 g, 8.99 mmol, 1.30 eq.) and TEA (1.04 g, 10.28 mmol, 1.49 eq.). The resulting mixture was stirred under N₂ at 80° C. overnight, cooled and then concentrated. The residue was purified by silica gel chromatography (DCM:MeOH=20:1) to give the title compound as a blue solid.

### Step 4: 2-((2-(2,6-Dioxopiperidin-3-yl)-1-oxoisoindolin-4-yl)oxy) ethyl 4-methylbenzenesulfonate

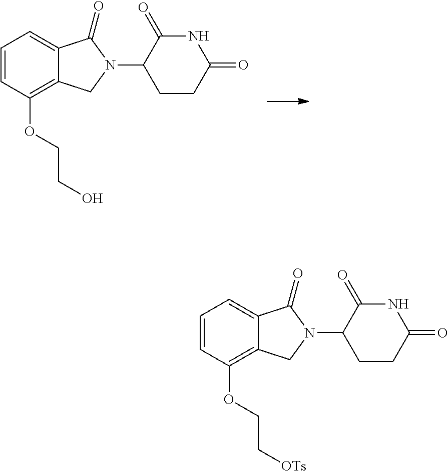 [SMILES: O=C1CCC(N2Cc3c(OCCO)cccc3C2=O)C(=O)N1 ; Cc1ccc(S(=O)(=O)OCCOc2cccc3c2CN(C2CCC(=O)NC2=O)C3=O)cc1]

To a stirred solution of 3-(4-(2-hydroxyethoxy)-1-oxoisoindolin-2-yl)piperidine-2,6-dione (500 mg, 1.64 mmol, 1.00 eq.) in DCM (10.0 mL) was added TEA (333 mg, 3.29 mmol, 2.00 eq.), TsCl (377 mg, 1.98 mmol, 1.21 eq.) and DMAP (20 mg, 0.16 mmol, 0.10 eq.) at 0° C. The resulting mixture was stirred at RT overnight, diluted with DCM, washed with water, brine, dried over Na₂SO₄, filtered, and then concentrated. The residue was purified by silica gel chromatography (DCM:MeOH=30:1) to give the title compound as a green solid.

### Step 5: Benzyl 4-((methylsulfonyl)oxy)piperidine-1-carboxylate

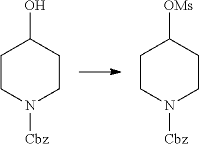 [SMILES: O=C(OCc1ccccc1)N1CCC(O)CC1 ; CS(=O)(=O)OC1CCN(C(=O)OCc2ccccc2)CC1]

To a stirred solution of benzyl 4-hydroxypiperidine-1-carboxylate (2.00 g, 8.50 mmol, 1.00 eq.) in DCM (20.0 mL) was added TEA (2.57 g, 25.40 mmol, 3.00 eq.) and MsCl (1.16 g, 10.13 mmol, 1.20 eq.) at 0° C. The resulting mixture was stirred at RT overnight, diluted with water and then extracted with DCM. The organic layer was washed with water, brine, dried over Na₂SO₄, filtered, and concentrated to give the crude title compound as a yellow oil, which was used for next step without further purification.

### Step 6: tert-Butyl (1-((3-(piperidin-4-yloxy)phenyl)sulfonyl)piperidin-4-yl)carbamate

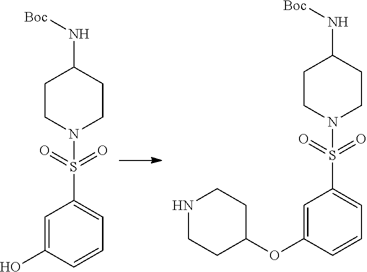 [SMILES: CC(C)(C)OC(=O)NC1CCN(S(=O)(=O)c2cccc(O)c2)CC1 ; CC(C)(C)OC(=O)NC1CCN(S(=O)(=O)c2cccc(OC3CCNCC3)c2)CC1]

Benzyl 4-((methylsulfonyl)oxy)piperidine-1-carboxylate was converted to the title compound by proceeding analogously as described in Reference 12, Steps 5-6 above.

### Step 7: tert-Butyl (1-((3-((1-(2-((2-(2,6-dioxopiperidin-3-yl)-1-oxoisoindolin-4-yl)oxy)ethyl)piperidin-4-yl)oxy)phenyl)sulfonyl)piperidin-4-yl)carbamate

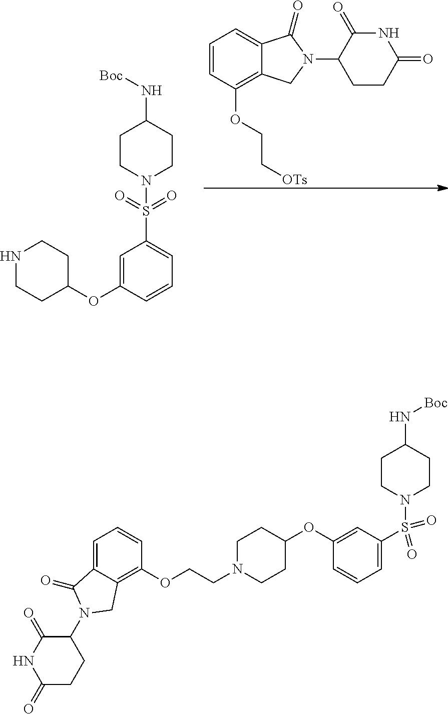 [SMILES: CC(C)(C)OC(=O)NC1CCN(S(=O)(=O)c2cccc(OC3CCNCC3)c2)CC1 ; Cc1ccc(S(=O)(=O)OCCOc2cccc3c2CN(C2CCC(=O)NC2=O)C3=O)cc1 ; CC(C)(C)OC(=O)NC1CCN(S(=O)(=O)c2cccc(OC3CCN(CCOc4cccc5c4CN(C4CCC(=O)NC4=O)C5=O)CC3)c2)CC1]

A mixture of of 2-((2-(2,6-dioxopiperidin-3-yl)-1-oxoisoindolin-4-yl)oxy)ethyl 4-methylbenzenesulfonate (50 mg, 0.11 mmol, 1.10 eq.), tert-butyl (1-((3-(piperidin-4-yloxy)phenyl)sulfonyl)piperidin-4-yl)carbamate (44 mg, 0.10 mmol, 1.00 eq.), KI (15 mg, 0.090 mmol, 0.90 eq.) and DIPEA (35 mg, 0.27 mmol, 2.70 eq.) in ACN (2.0 mL) was stirred at 100° C. under microwave for 3 h. The reaction mixture was cooled and concentrated, and the residue was purified by silica gel chromatography (DCM:MeOH=20:1) to give the title as a yellow oil.

### Reference 26

### Synthesis of tert-butyl (1-((3-((1-(2-((2-(2,6-dioxopiperidin-3-yl)-1-oxoisoindolin-4-yl)oxy)-ethyl)azetidin-3-yl)oxy)phenyl)sulfonyl)piperidin-4-yl)carbamate

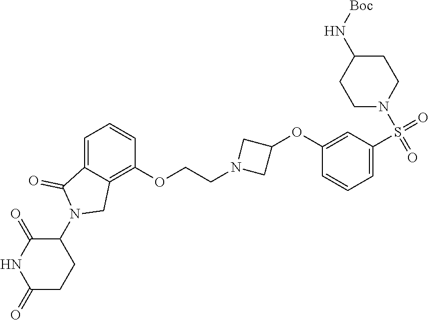 [SMILES: CC(C)(C)OC(=O)NC1CCN(S(=O)(=O)c2cccc(OC3CN(CCOc4cccc5c4CN(C4CCC(=O)NC4=O)C5=O)C3)c2)CC1]

tert-Butyl (1-((3-(azetidin-3-yloxy)phenyl)sulfonyl)piperidin-4-yl)carbamate was converted to the title compound by proceeding analogously as described in Reference 25, Step 7.

### Reference 27

### Synthesis of tert-butyl (1-((3-((1-(2-(2,6-dioxopiperidin-3-yl)-1-oxoisoindolin-5-yl)piperidin-4-yl)oxy)phenyl)sulfonyl)piperidin-4-yl)carbamate

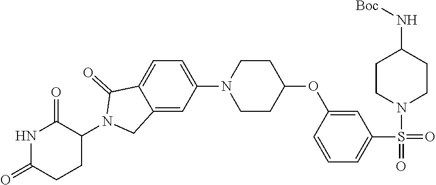 [SMILES: CC(C)(C)OC(=O)NC1CCN(S(=O)(=O)c2cccc(OC3CCN(c4ccc5c(c4)CN(C4CCC(=O)NC4=O)C5=O)CC3)c2)CC1]

A mixture of tert-butyl (1-((3-(piperidin-4-yloxy)phenyl)sulfonyl)piperidin-4-yl)carbamate (300 mg, 0.93 mmol, 1.00 eq.), 3-(5-bromo-1-oxoisoindolin-2-yl)piperidine-2,6-dione (448 mg, 1.02 mmol, 1.10 eq.), Cs₂CO₃ (603 mg, 1.86 mmol, 2.00 eq.), Pd(OAc)₂ (41 mg, 0.19 mmol, 0.20 eq.) and X-Phos (176 mg, 0.37 mmol, 0.40 eq.) in 1,4-dioxane (10.0 mL) was stirred at 105° C. under N₂ for 2 days. The reaction mixture was diluted with water and extracted with DCM. The combined organic layer was washed with brine and dried over Na₂SO₄ and concentrated. The residue was purified by flash chromatography gave title compound as a yellow solid.

### Reference 28

### Synthesis of tert-butyl (1-((3-(1-((1-(2-(2,6-dioxopiperidin-3-yl)-1-oxoisoindolin-5-yl)-piperidin-4-yl)methyl)piperidin-4-yl)phenyl)sulfonyl)piperidin-4-yl)carbamate

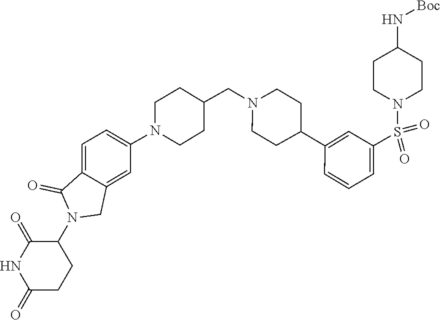 [SMILES: CC(C)(C)OC(=O)NC1CCN(S(=O)(=O)c2cccc(C3CCN(CC4CCN(c5ccc6c(c5)CN(C5CCC(=O)NC5=O)C6=O)CC4)CC3)c2)CC1]

### Step 1: Benzyl 4-(3-((4-((tert-butoxycarbonyl)amino)piperidin-1-yl)sulfonyl)phenyl)-5,6-dihydropyridine-1(2H)-carboxylate

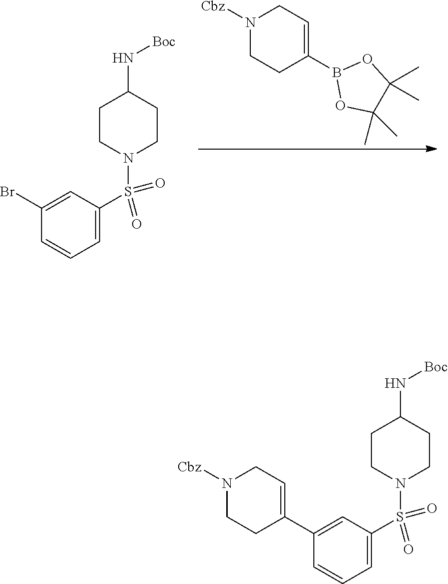 [SMILES: CC(C)(C)OC(=O)NC1CCN(S(=O)(=O)c2cccc(Br)c2)CC1 ; CC1(C)OB(C2=CCN(C(=O)OCc3ccccc3)CC2)OC1(C)C ; CC(C)(C)OC(=O)NC1CCN(S(=O)(=O)c2cccc(C3=CCN(C(=O)OCc4ccccc4)CC3)c2)CC1]

A mixture of tert-butyl (1-((3-bromophenyl)sulfonyl)piperidin-4-yl)carbamate (3.00 g, 7.18 mmol, 1.00 eq.), benzyl 4-(4,4,5,5-tetramethyl-1,3,2-dioxaborolan-2-yl)-5,6-dihydropyridine-1(2H)-carboxylate (3.20 g, 9.33 mmol, 1.30 eq.), X-Phos-Pd-G3 (608.0 mg, 0.72 mmol, 0.10 eq.) and K₃PO₄ (4.57 g, 21.54 mmol, 3.00 eq.) in 1,4-dioxane (70.0 mL) and H₂O (7.0 mL) was stirred at 60° C. under N₂ for 6 h. The resulting mixture was concentrated and the residue was purified by silica gel column chromatography, eluted with PE/EtOAc (4:1), to afford the title compound as a yellow solid.

### Step 2: tert-Butyl (1-((3-(piperidin-4-yl)phenyl)sulfonyl)piperidin-4-yl)carbamate

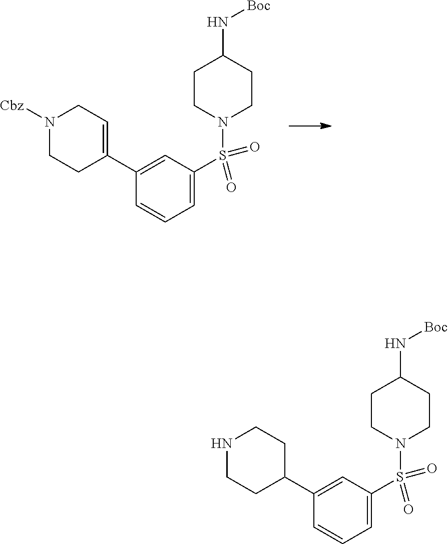 [SMILES: CC(C)(C)OC(=O)NC1CCN(S(=O)(=O)c2cccc(C3=CCN(C(=O)OCc4ccccc4)CC3)c2)CC1 ; CC(C)(C)OC(=O)NC1CCN(S(=O)(=O)c2cccc(C3CCNCC3)c2)CC1]

A mixture of benzyl 4-(3-((4-((tert-butoxycarbonyl)amino)piperidin-1-yl)sulfonyl)phenyl)-5,6-dihydropyridine-1(2H)-carboxylate (4.00 g, 7.20 mmol, 1.00 eq.) and Pd/C (800 mg) in MeOH (40.0 mL) was stirred at 50° C. under H₂ (50 psi) for 16 h. The mixture was filtered and concentrated to afford the title compound as a white solid.

### Step 3: Benzyl 4-((4-(3-((4-((tert-butoxycarbonyl)amino)piperidin-1-yl)sulfonyl)phenyl)piperidin-1-yl)methyl)piperidine-1-carboxylate

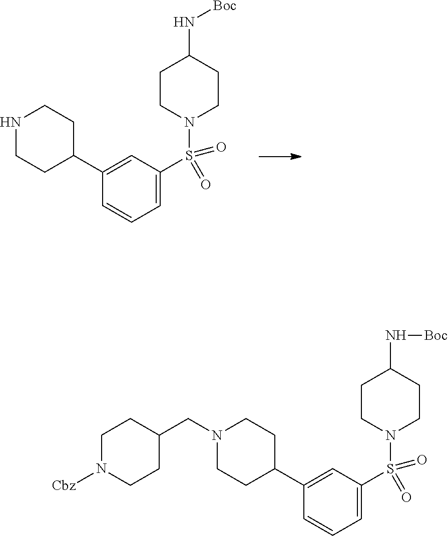 [SMILES: CC(C)(C)OC(=O)NC1CCN(S(=O)(=O)c2cccc(C3CCNCC3)c2)CC1 ; CC(C)(C)OC(=O)NC1CCN(S(=O)(=O)c2cccc(C3CCN(CC4CCN(C(=O)OCc5ccccc5)CC4)CC3)c2)CC1]

To a solution of tert-butyl (1-((3-(piperidin-4-yl)phenyl)sulfonyl)piperidin-4-yl)carbamate (3.00 g, 7.10 mmol, 1.00 eq.) in DCE (20.0 mL) and MeOH (20.0 mL) was added benzyl 4-formylpiperidine-1-carboxylate (2.63 g, 10.65 mmol, 1.50 eq.) and AcOH (426.0 mg, 7.10 mmol, 1.00 eq.) and the solution was stirred at RT for 1 h. NaBH₃CN (1.34 g, 21.30 mmol, 3.00 eq.) was added and the mixture was stirred at RT for 3 h. The resulting mixture was concentrated and the residue was purified by silica gel column chromatography, eluted with DCM/MeOH (60:1), to afford the title compound as a white solid.

### Step 4: tert-Butyl (1-((3-(1-(piperidin-4-ylmethyl)piperidin-4-yl)phenyl)sulfonyl)piperidin-4-yl)-carbamate

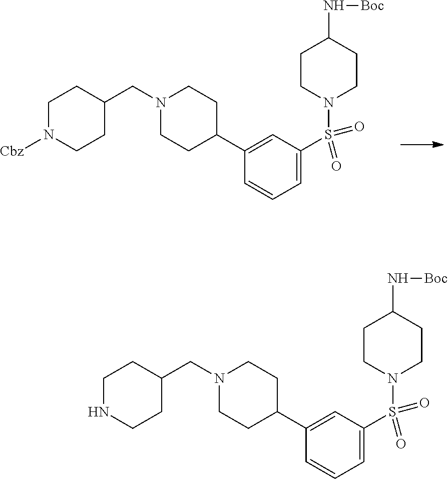 [SMILES: CC(C)(C)OC(=O)NC1CCN(S(=O)(=O)c2cccc(C3CCN(CC4CCN(C(=O)OCc5ccccc5)CC4)CC3)c2)CC1 ; CC(C)(C)OC(=O)NC1CCN(S(=O)(=O)c2cccc(C3CCN(CC4CCNCC4)CC3)c2)CC1]

A mixture of benzyl 4-((4-(3-((4-((tert-butoxycarbonyl)amino)piperidin-1-yl)-sulfonyl)phenyl)piperidin-1-yl)methyl)piperidine-1-carboxylate (3.80 g, 5.81 mmol, 1.00 eq.) and Pd/C (800 mg) in MeOH (40.0 mL) was stirred at 50° C. under H₂ (50 psi) for 16 h. The mixture was filtered and concentrated to afford the title compound as a white solid.

### Step 5: Methyl 4-(4-((4-(3-((4-((tert-butoxycarbonyl)amino)piperidin-1-yl)sulfonyl)-phenyl)piperidin-1-yl)methyl)piperidin-1-yl)-2-cyanobenzoate

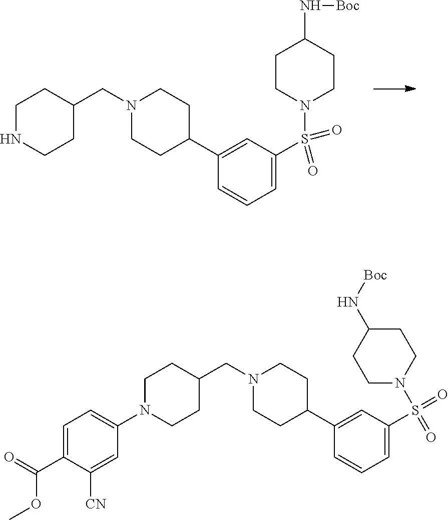 [SMILES: CC(C)(C)OC(=O)NC1CCN(S(=O)(=O)c2cccc(C3CCN(CC4CCNCC4)CC3)c2)CC1 ; COC(=O)c1ccc(N2CCC(CN3CCC(c4cccc(S(=O)(=O)N5CCC(NC(=O)OC(C)(C)C)CC5)c4)CC3)CC2)cc1C#N]

A solution of tert-butyl (1-((3-(1-(piperidin-4-ylmethyl)piperidin-4-yl)phenyl)-sulfonyl)piperidin-4-yl)carbamate (2.80 g, 5.40 mmol, 1.00 eq.), methyl 2-cyano-4-fluorobenzoate (1.06 g, 5.94 mmol, 1.10 eq.) and DIEA (2.09 g, 16.20 mmol, 3.00 eq.) in DMSO (30.0 mL) was stirred at 120° C. under N₂ for 16 h. The mixture was cooled to RT, diluted with water, and then extracted with EtOAc. The combined organic layer was washed with water, dried over anhydrous Na₂SO₄, filtered, and then concentrated. The residue was purified by silica gel column chromatography, eluted with DCM/MeOH (100:1), to afford the title compound as a brown solid.

### Step 6: Methyl 4-(4-((4-(3-((4-((tert-butoxycarbonyl)amino)piperidin-1-yl)sulfonyl)-phenyl)piperidin-1-yl)methyl)piperidin-1-yl)-2-formylbenzoate

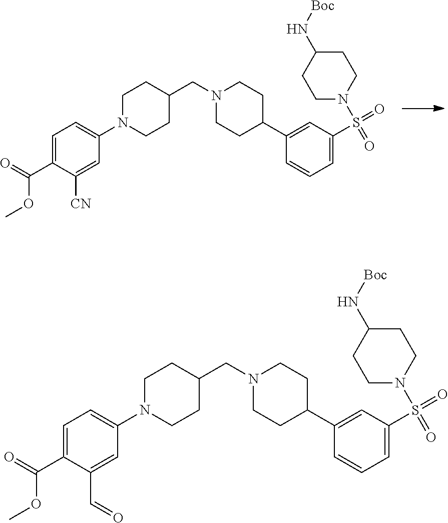 [SMILES: COC(=O)c1ccc(N2CCC(CN3CCC(c4cccc(S(=O)(=O)N5CCC(NC(=O)OC(C)(C)C)CC5)c4)CC3)CC2)cc1C#N ; COC(=O)c1ccc(N2CCC(CN3CCC(c4cccc(S(=O)(=O)N5CCC(NC(=O)OC(C)(C)C)CC5)c4)CC3)CC2)cc1C=O]

A mixture of methyl 4-(4-((4-(3-((4-((tert-butoxycarbonyl)amino)piperidin-1-yl)-sulfonyl)phenyl)piperidin-1-yl)methyl)piperidin-1-yl)-2-cyanobenzoate (1.01 g, 1.50 mmol, 1.00 eq.), NaH₂PO₂·H₂O (1.59 g, 15.00 mmol, 10.00 eq.) and Raney Ni (1.60 g) in pyridine (10.0 mL), H₂O (5.0 mL) and AcOH (5.0 mL) was stirred for 16 h at 70° C. under nitrogen atmosphere. The resulting mixture was diluted with EtOAc and washed with water, brine, dried over anhydrous Na₂SO₄ and concentrated. The residue was purified by silica gel column chromatography, eluted with DCM/MeOH (80:1), to afford the title compound as a light-yellow solid.

### Step 7: tert-Butyl (1-((3-(1-((1-(2-(2,6-dioxopiperidin-3-yl)-1-oxoisoindolin-5-yl)piperidin-4-yl)methyl)piperidin-4-yl)phenyl)sulfonyl)piperidin-4-yl)carbamate

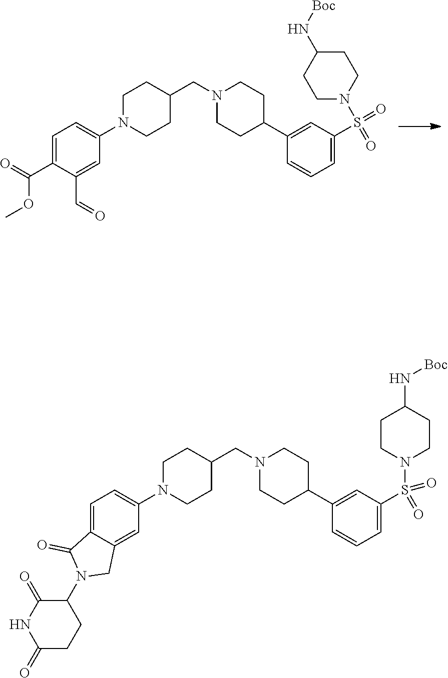 [SMILES: COC(=O)c1ccc(N2CCC(CN3CCC(c4cccc(S(=O)(=O)N5CCC(NC(=O)OC(C)(C)C)CC5)c4)CC3)CC2)cc1C=O ; CC(C)(C)OC(=O)NC1CCN(S(=O)(=O)c2cccc(C3CCN(CC4CCN(c5ccc6c(c5)CN(C5CCC(=O)NC5=O)C6=O)CC4)CC3)c2)CC1]

A mixture of 3-aminopiperidine-2,6-dione hydrochloride (126 mg, 0.77 mmol, 1.30 eq.) and DIEA (184 mg, 1.43 mmol, 2.40 eq.) in dry DCM (5.0 mL) was stirred at RT for 10 min and then a solution of methyl 4-(4-((4-(3-((4-((tert-butoxycarbonyl)amino)piperidin-1-yl)sulfonyl)phenyl)piperidin-1-yl)methyl)piperidin-1-yl)-2-formylbenzoate (400 mg, 0.59 mmol, 1.00 eq.) in dry DCM (5.0 mL) and AcOH (134 mg, 2.23 mmol, 3.80 eq.) was added. The mixture was stirred at 45° C. under N₂ for 3 h. The mixture was cooled to 0° C. and NaBH(OAc)₃ (375 mg, 1.77 mmol, 3.00 eq.) was added. The mixture was stirred at rt for 1 h and then at 45° C. under N₂ for 16 h. The mixture was cooled, diluted with water, and then extracted with DCM. The combined organic layer was washed with water, dried over anhydrous Na₂SO₄, filtered, and concentrated. The residue was purified by silica gel column chromatography, eluted with DCM/MeOH (40:1), to afford the title compound as a yellow solid.

### Reference 29

### Synthesis of tert-butyl (1-((3-(4-((4-(2-(2,6-dioxopiperidin-3-yl)-1-oxoisoindolin-5-yl)piperidin-1-yl)methyl)piperidin-1-yl)phenyl)sulfonyl)piperidin-4-yl)carbamate

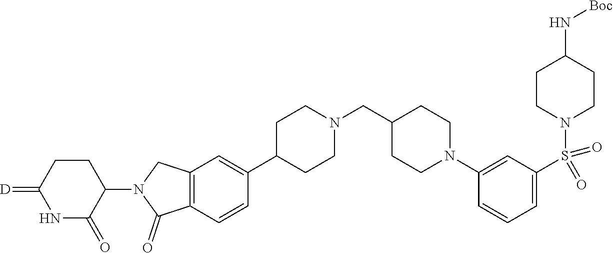

### Step 1: 3-5-Bromo-1-oxoisoindolin-2-ylpiperidine-2,6-dione

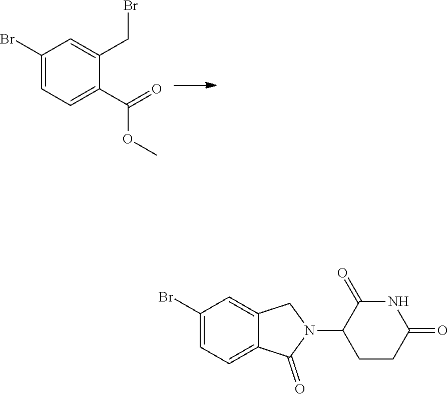 [SMILES: COC(=O)c1ccc(Br)cc1CBr ; O=C1CCC(N2Cc3cc(Br)ccc3C2=O)C(=O)N1]

A mixture of methyl 4-bromo-2-(bromomethyl)benzoate (20.00 g, 64.91 mmol, 1.00 eq.) and 3-aminopiperidine-2,6-dione (11.71 g, 71.41 mmol, 1.10 eq.), K₂CO₃ (26.87 g, 194.71 mmol, 3.00 eq.) in DMF was stirred at 70° C. overnight under N₂ atmosphere. The mixture was poured into water after the reaction was complete and extracted with DCM. The combined organic layer was washed with water, dried over anhydrous Na₂SO₄, filtered, and concentrated. The residue was purified by flash column (PE:EA=2:1) to give the title compound as a white solid.

### Step 2: tert-Butyl 4-(2-(2,6-dioxopiperidin-3-yl)-1-oxoisoindolin-5-yl)-5,6-dihydropyridine-1(2H)-carboxylate

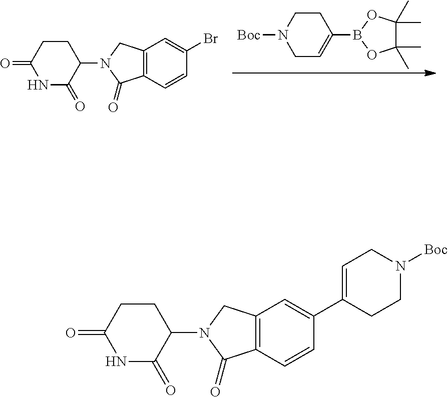 [SMILES: O=C1CCC(N2Cc3cc(Br)ccc3C2=O)C(=O)N1 ; CC(C)(C)OC(=O)N1CC=C(B2OC(C)(C)C(C)(C)O2)CC1 ; CC(C)(C)OC(=O)N1CC=C(c2ccc3c(c2)CN(C2CCC(=O)NC2=O)C3=O)CC1]

A mixture of 3-(5-bromo-1-oxoisoindolin-2-yl)piperidine-2,6-dione (1.00 g, 3.11 mmol, 1.00 eq.), tert-butyl 4-(4,4,5,5-tetramethyl-1,3,2-dioxaborolan-2-yl)-5,6-dihydropyridine-1(2H)-carboxylate (1.25 g, 4.04 mmol, 1.30 eq.), K₃PO₄ (800 mg, 3.73 mmol, 1.20 eq) and Pd(dppf)Cl₂ (114 mg, 0.16 mmol, 0.05 eq) in DMF (10.0 mL) was stirred at 90° C. for 12 h. The mixture was concentrated and purified by silica gel column chromatography eluting with PE/EA (1:2) to give title compound as yellow solid.

### Step 3: tert-Butyl 4-(2-(2,6-dioxopiperidin-3-yl)-1-oxoisoindolin-5-yl)piperidine-1-carboxylate

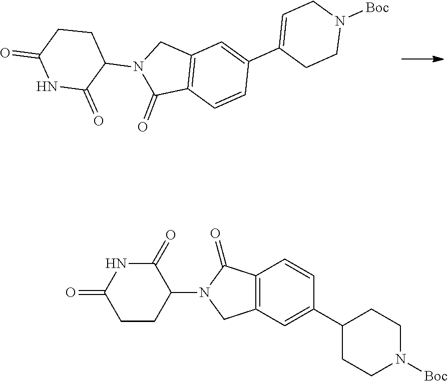 [SMILES: CC(C)(C)OC(=O)N1CC=C(c2ccc3c(c2)CN(C2CCC(=O)NC2=O)C3=O)CC1 ; CC(C)(C)OC(=O)N1CCC(c2ccc3c(c2)CN(C2CCC(=O)NC2=O)C3=O)CC1]

To a stirred solution of tert-butyl 4-(2-(2,6-dioxopiperidin-3-yl)-1-oxoisoindolin-5-yl)-5,6-dihydropyridine-1(2H)-carboxylate (200 mg, 0.47 mmol, 1.00 eq.) in THF (2.0 mL) was added Pd/C (40 mg, 20% w/w). The resulting mixture was stirred at 40° C. for 12 h under H₂, filtered and concentrated to give the title compound as white solid.

### Step 4: 3-(1-Oxo-5-(piperidin-4-yl) isoindolin-2-yl)piperidine-2,6-dione

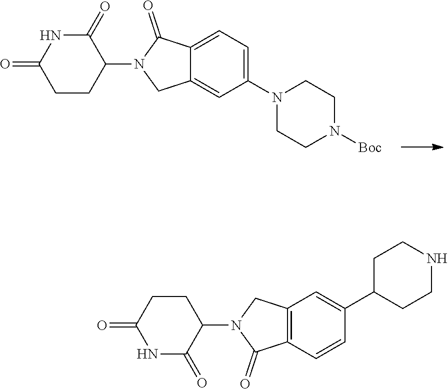 [SMILES: CC(C)(C)OC(=O)N1CCN(c2ccc3c(c2)CN(C2CCC(=O)NC2=O)C3=O)CC1 ; O=C1CCC(N2Cc3cc(C4CCNCC4)ccc3C2=O)C(=O)N1]

DCM/TFA=4:1 (2.5 mL) was added to a stirred solution of tert-butyl 4-(2-(2,6-dioxopiperidin-3-yl)-1-oxoisoindolin-5-yl)piperidine-1-carboxylate (100 mg, 0.234 mmol, 1.00 eq.) and the mixture was stirred at RT for 2 h. The mixture was concentrated to give the title compound as brown solid.

### Step 5: tert-Butyl (1-((3-(4-((4-(2-(2,6-dioxopiperidin-3-yl)-1-oxoisoindolin-5-yl)piperidin-1-yl)methyl)piperidin-1-yl)phenyl)sulfonyl)piperidin-4-yl)carbamate

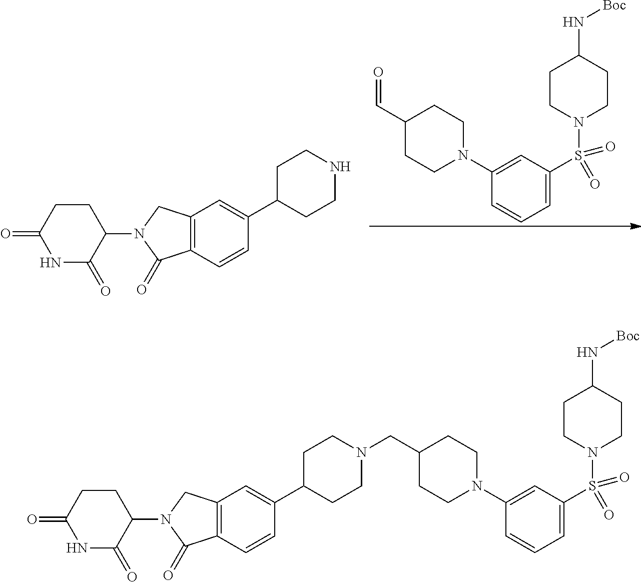 [SMILES: O=C1CCC(N2Cc3cc(C4CCNCC4)ccc3C2=O)C(=O)N1 ; CC(C)(C)OC(=O)NC1CCN(S(=O)(=O)c2cccc(N3CCC(C=O)CC3)c2)CC1 ; CC(C)(C)OC(=O)NC1CCN(S(=O)(=O)c2cccc(N3CCC(CN4CCC(c5ccc6c(c5)CN(C5CCC(=O)NC5=O)C6=O)CC4)CC3)c2)CC1]

To a stirred solution of 3-(1-oxo-5-(piperidin-4-yl) isoindolin-2-yl)piperidine-2,6-dione (76.60 mg, 0.23 mmol, 1.00 eq) in THF (1.0 mL) was added DMF (1.0 mL), HCOOH (1 drop) and tert-butyl (1-((3-(4-formylpiperidin-1-yl)phenyl)sulfonyl)piperidin-4-yl)carbamate (105.60 mg, 0.23 mmol 1.00 eq). The resulting mixture was stirred at 45° C. for 0.5 h. NaBH₃CN (29.40 mg, 0.47 mmol, 2.00 eq) was added at RT and the reaction mixture was stirred at RT for 12 h. The mixture was diluted with water and extracted with EtOAc. The combined organic layer was washed with water and brine and concentrated. The residue was purified by silica gel column chromatography, eluted with DCM:MeOH (0˜100%), to give the title compound as a white solid.

### Reference 30

### Synthesis of tert-butyl (1-((3-(4-((4-(2-(2,6-dioxopiperidin-3-yl)-1-oxoisoindolin-5-yl)piperazin-1-yl)-methyl)piperidin-1-yl)phenyl)sulfonyl)piperidin-4-yl)carbamate

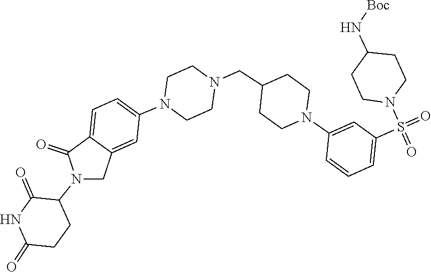 [SMILES: CC(C)(C)OC(=O)NC1CCN(S(=O)(=O)c2cccc(N3CCC(CN4CCN(c5ccc6c(c5)CN(C5CCC(=O)NC5=O)C6=O)CC4)CC3)c2)CC1]

### Step 1: Benzyl 4-(dimethoxymethyl)piperidine-1-carboxylate

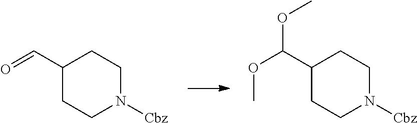 [SMILES: O=CC1CCN(C(=O)OCc2ccccc2)CC1 ; COC(OC)C1CCN(C(=O)OCc2ccccc2)CC1]

To a mixture of benzyl 4-formylpiperidine-1-carboxylate (1.00 g, 2.40 mmol, 1.00 eq.) in MeOH (9.0 mL) was added p-TsOH (38 mg, 0.20 mmol, 0.05 eq.) and trimethoxymethane (2.14 g, 20.22 mmol, 5.00 eq.). The mixture was stirred at RT for 12 h and then extracted with EtOAc. Purification of the crude product by silica gel column chromatography eluting with PE/EtOAc (10:1) gave the title compound as a colorless oil.

### Step 2: 4-(Dimethoxymethyl)piperidine

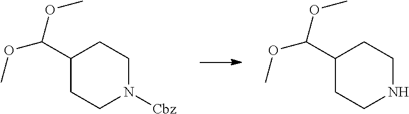 [SMILES: COC(OC)C1CCN(C(=O)OCc2ccccc2)CC1 ; COC(OC)C1CCNCC1]

To a mixture of benzyl 4-(dimethoxymethyl)piperidine-1-carboxylate (948 mg, 3.23 mmol, 1.00 eq.) in MeOH (10.0 mL) was added Pd/C (400 mg) and the reaction mixture was stirred at RT under H₂ for overnight. The resulting mixture was filtered through Celite and the filtrate was concentrated to give the title compound as a colorless oil.

### Step 3: tert-Butyl (1-((3-(4-(dimethoxymethyl)piperidin-1-yl)phenyl)sulfonyl)piperidin-4-yl)-carbamate

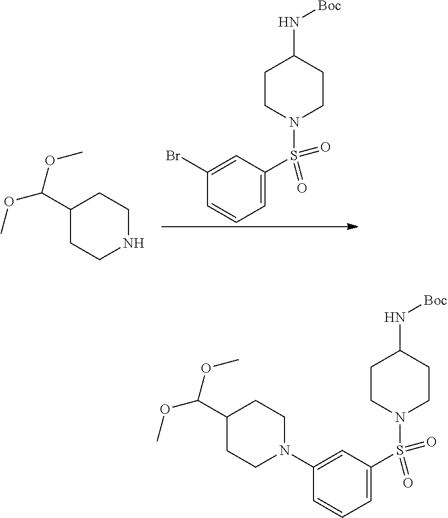 [SMILES: COC(OC)C1CCNCC1 ; CC(C)(C)OC(=O)NC1CCN(S(=O)(=O)c2cccc(Br)c2)CC1 ; COC(OC)C1CCN(c2cccc(S(=O)(=O)N3CCC(NC(=O)OC(C)(C)C)CC3)c2)CC1]

A mixture of 4-(dimethoxymethyl)piperidine (100 mg, 0.63 mmol, 1.20 eq.), K₂CO₃ (215 mg, 1.56 mmol, 3.00 eq.), CuI (20 mg, 0.104 mmol, 0.20 eq.), L-proline (18 mg, 0.16 mmol, 0.30 eq.) and tert-butyl (1-((3-bromophenyl)sulfonyl)piperidin-4-yl)carbamate (219 mg, 0.52 mmol, 1.00 eq.) in DMSO (4.0 mL) was stirred at 90° C. overnight. The reaction mixture was diluted with water and extracted with EtOAc. The combined organic layer was washed with water and brine and concentrated. The residue was purified by silica gel column chromatography eluting with PE/EtOAc (1:1) to give the title compound as white solid.

### Step 4: tert-Butyl (1-((3-(4-formylpiperidin-1-yl)phenyl)sulfonyl)piperidin-4-yl)carbamate

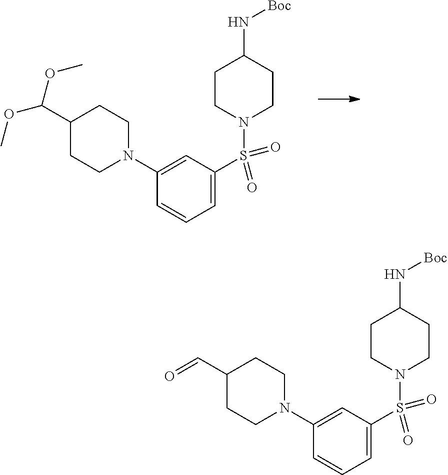 [SMILES: COC(OC)C1CCN(c2cccc(S(=O)(=O)N3CCC(NC(=O)OC(C)(C)C)CC3)c2)CC1 ; CC(C)(C)OC(=O)NC1CCN(S(=O)(=O)c2cccc(N3CCC(C=O)CC3)c2)CC1]

To a mixture of tert-butyl (1-((3-(4-(dimethoxymethyl)piperidin-1-yl)phenyl)sulfonyl) piperidin-4-yl)carbamate (640 mg, 1.29 mmol, 1.00 eq.) in DCM (4.0 mL) was added TFA (4.0 mL) and the mixture was stirred at 45° C. overnight. The reaction mixture was concentrated and dissolved in DCM (5.0 mL) followed by addition of TEA (261 mg, 2.58 mmol, 2.00 eq.) and (Boc)₂O (562 mg, 2.58 mmol, 2.00 eq.). The solution was stirred at RT for 4 h, and concentrated and the residue was purified by silica gel column chromatography eluting with PE/EtOAc (3:1) to give title compound as yellow solid.

### Step 5: tert-Butyl (1-((3-(4-((4-(2-(2,6-dioxopiperidin-3-yl)-1-oxoisoindolin-5-yl)piperazin-1-yl)-methyl)piperidin-1-yl)phenyl)sulfonyl)piperidin-4-yl)carbamate

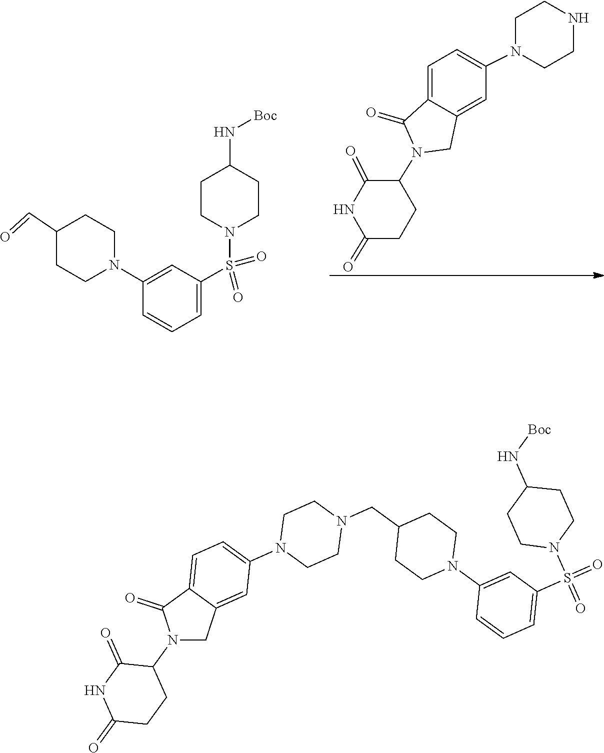 [SMILES: CC(C)(C)OC(=O)NC1CCN(S(=O)(=O)c2cccc(N3CCC(C=O)CC3)c2)CC1 ; O=C1CCC(N2Cc3cc(N4CCNCC4)ccc3C2=O)C(=O)N1 ; CC(C)(C)OC(=O)NC1CCN(S(=O)(=O)c2cccc(N3CCC(CN4CCN(c5ccc6c(c5)CN(C5CCC(=O)NC5=O)C6=O)CC4)CC3)c2)CC1]

The compound was prepared analogously as described in Reference 29, Step 5.

### Reference 31

### Synthesis of 3-(5-(azetidin-3-yl)-1-oxoisoindolin-2-yl)piperidine-2,6-dione

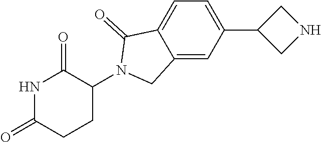 [SMILES: O=C1CCC(N2Cc3cc(C4CNC4)ccc3C2=O)C(=O)N1]

### Step 1:(1-(tert-Butoxycarbonyl)azetidin-3-yl)zinc(II) iodide

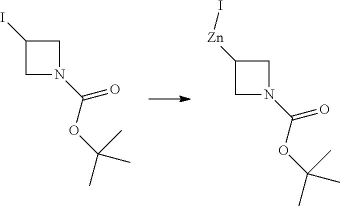

To a mixture of Zn dust (300 mg, 4.59 mmol, 1.30 eq.) in DMA (3.0 mL) was added 1,2-dibromoethene (66 mg, 0.35 mmol, 0.10 eq.) and the mixture was stirred at 65° C. under N₂ for 30 min. The mixture was allowed to cool to RT and TMSCl (38 mg, 0.35 mmol, 0.10 eq.) was added. After stirring the mixture for 30 min., a solution of tert-butyl 3-iodoazetidine-1-carboxylate (1.00 g, 3.53 mmol, 1.00 eq.) in DMA (1.0 mL) was added dropwise. The mixture was stirred at 65° C. under N₂ for 2 h, cooled to RT and used in next step without further purification.

### Step 2: tert-Butyl 3-(2-(2,6-dioxopiperidin-3-yl)-1-oxoisoindolin-5-yl)azetidine-1-carboxylate

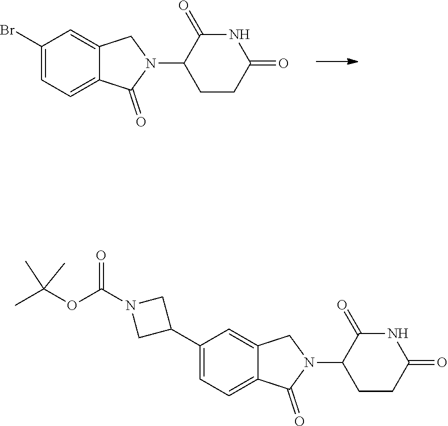 [SMILES: O=C1CCC(N2Cc3cc(Br)ccc3C2=O)C(=O)N1 ; CC(C)(C)OC(=O)N1CC(c2ccc3c(c2)CN(C2CCC(=O)NC2=O)C3=O)C1]

A solution of (1-(tert-butoxycarbonyl)azetidin-3-yl)zinc(II) iodide (600 mg, 1.72 mmol, 3.00 eq.) in DMA was slowly added to a mixture of 3-(5-bromo-1-oxoisoindolin-2-yl)piperidine-2,6-dione (185 mg, 0.57 mmol, 1.00 eq.), CuI (12 mg, 0.06 mmol, 0.10 eq.), Pd(dppf)Cl₂ (44 mg, 0.06 mmol, 0.10 eq.) in DMA (2.0 mL). The mixture was stirred at 90° C. under N₂ overnight. The mixture was concentrated and purified by column chromatography on silica gel (EA) to give the title compound as a brown solid.

### Step 3: 3-(5-(Azetidin-3-yl)-1-oxoisoindolin-2-yl)piperidine-2,6-dione

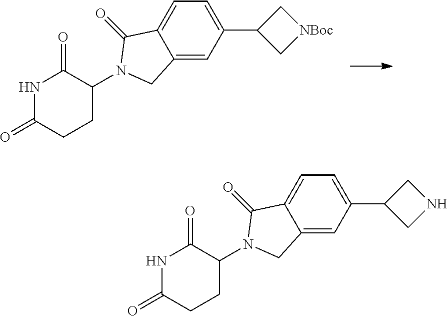 [SMILES: CC(C)(C)OC(=O)N1CC(c2ccc3c(c2)CN(C2CCC(=O)NC2=O)C3=O)C1 ; O=C1CCC(N2Cc3cc(C4CNC4)ccc3C2=O)C(=O)N1]

To a solution of tert-butyl 3-(2-(2,6-dioxopiperidin-3-yl)-1-oxoisoindolin-5-yl)azetidine-1-carboxylate (44 mg, 0.11 mmol, 1.00 eq.) in DCM (1.0 mL) was added TFA (0.2 mL) dropwise and the solution was stirred at RT for 3 h. The resulting mixture was concentrated to give the crude product as a brown oil.

### Reference 32

### Synthesis of tert-butyl (1-((3-(4-((1-(2-(2,6-dioxopiperidin-3-yl)-1-oxoisoindolin-5-yl)piperidin-4-yl)methyl)piperazin-1-yl)phenyl)sulfonyl)piperidin-4-yl)carbamate

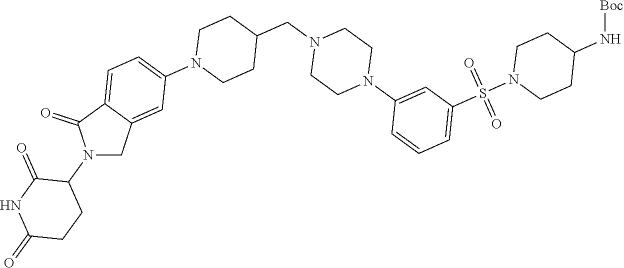 [SMILES: CC(C)(C)OC(=O)NC1CCN(S(=O)(=O)c2cccc(N3CCN(CC4CCN(c5ccc6c(c5)CN(C5CCC(=O)NC5=O)C6=O)CC4)CC3)c2)CC1]

### Step 1: Benzyl 4-(3-((4-((tert-butoxycarbonyl)amino)piperidin-1-yl)sulfonyl)phenyl)piperazine-1-carboxylate

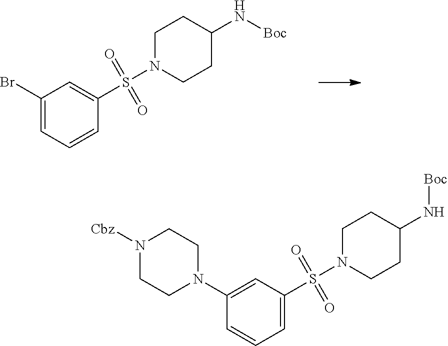 [SMILES: CC(C)(C)OC(=O)NC1CCN(S(=O)(=O)c2cccc(Br)c2)CC1 ; CC(C)(C)OC(=O)NC1CCN(S(=O)(=O)c2cccc(N3CCN(C(=O)OCc4ccccc4)CC3)c2)CC1]

A mixture of tert-butyl (1-((3-bromophenyl)sulfonyl)piperidin-4-yl)carbamate (5.00 g, 11.96 mmol, 1.00 eq.), K₂CO₃ (5.78 g, 41.86 mmol, 3.50 eq.), CuI (0.45 g, 2.39 mmol, 0.20 eq.), L-PRO (0.41 g, 3.59 mmol, 0.30 eq.) in DMSO (25.00 mL) and benzyl piperazine-1-carboxylate (3.43 g, 15.55 mmol, 1.30 eq.) was stirred at 100° C. for 12 h. The mixture was quenched with H₂O and extracted with EtOAc. The organic layer was concentrated and the residue was purified by silica gel column chromatography eluting with PE/EtOAc (3:1) to give the title compound as white solid.

### Step 2: tert-Butyl (1-((3-(piperazin-1-yl)phenyl)sulfonyl)piperidin-4-yl)carbamate

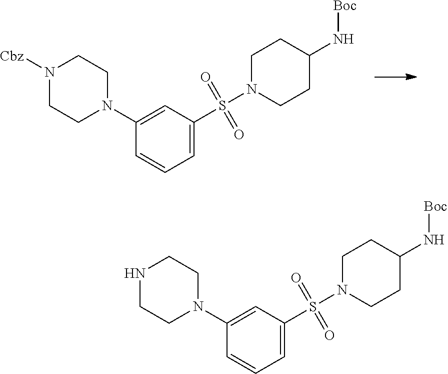 [SMILES: CC(C)(C)OC(=O)NC1CCN(S(=O)(=O)c2cccc(N3CCN(C(=O)OCc4ccccc4)CC3)c2)CC1 ; CC(C)(C)OC(=O)NC1CCN(S(=O)(=O)c2cccc(N3CCNCC3)c2)CC1]

The title compound was prepared analogously as described in Reference 30, Step 2.

### Step 3: Benzyl 4-((4-(3-((4-((tert-butoxycarbonyl)amino)piperidin-1-yl)sulfonyl)phenyl)piperazin-1-yl)methyl)piperidine-1-carboxylate

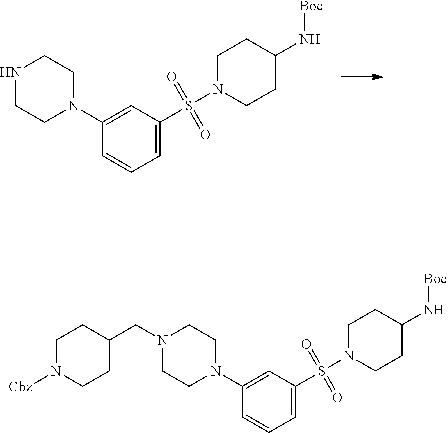 [SMILES: CC(C)(C)OC(=O)NC1CCN(S(=O)(=O)c2cccc(N3CCNCC3)c2)CC1 ; CC(C)(C)OC(=O)NC1CCN(S(=O)(=O)c2cccc(N3CCN(CC4CCN(C(=O)OCc5ccccc5)CC4)CC3)c2)CC1]

A mixture of tert-butyl (1-((3-(piperazin-1-yl)phenyl)sulfonyl)piperidin-4-yl)carbamate (1.07 g, 2.52 mmol, 1.00 eq.), AcOH (3 drops) and benzyl 4-formylpiperidine-1-carboxylate (933 mg, 3.78 mmol, 1.50 eq.) in MeOH (10.0 mL) was stirred at 45° C. for 1 h. The solution cooled to RT and NaBH₃CN (475 mg, 7.56 mmol, 3.00 eq.) was added. The mixture was stirred at RT for 12 h and then diluted with water and extracted with EtOAc. The combined organic layer was washed with brine, dried over anhydrous Na₂SO₄, and concentrated. The residue was purified by silica gel column chromatography eluting with DCM/MeOH (60:1) to give the title compound as white solid.

### Step 4: tert-Butyl (1-((3-(4-((1-(2-(2,6-dioxopiperidin-3-yl)-1-oxoisoindolin-5-yl)piperidin-4-yl)methyl)piperazin-1-yl)phenyl)sulfonyl)piperidin-4-yl)carbamate

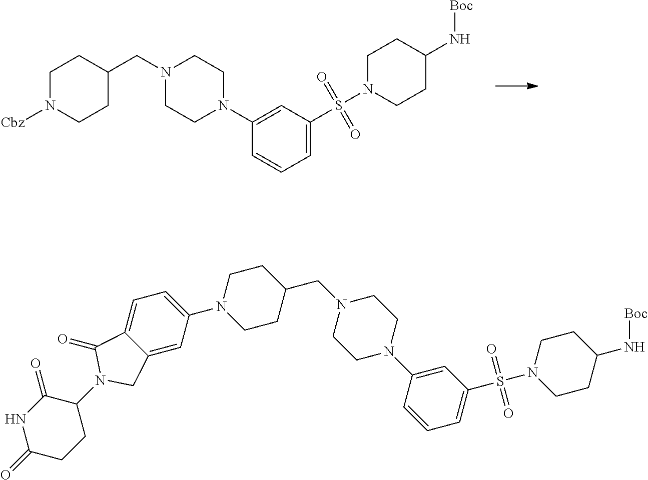 [SMILES: CC(C)(C)OC(=O)NC1CCN(S(=O)(=O)c2cccc(N3CCN(CC4CCN(C(=O)OCc5ccccc5)CC4)CC3)c2)CC1 ; CC(C)(C)OC(=O)NC1CCN(S(=O)(=O)c2cccc(N3CCN(CC4CCN(c5ccc6c(c5)CN(C5CCC(=O)NC5=O)C6=O)CC4)CC3)c2)CC1]

Benzyl 4-((4-(3-((4-((tert-butoxycarbonyl)amino)piperidin-1-yl)sulfonyl)phenyl)piperazin-1-yl)methyl)piperidine-1-carboxylate was converted to the title compound using similar procedure as described in Reference 28, Step 4-7.

### Reference 33

### Synthesis of tert-butyl (1-((3-(4-(1-(2,6-dioxopiperidin-3-yl)-3-methyl-2-oxo-2,3-dihydro-1H-benzo[d]imidazol-4-yl)-[1,4′-bipiperidin]-1′-yl)phenyl)sulfonyl)piperidin-4-yl)carbamate

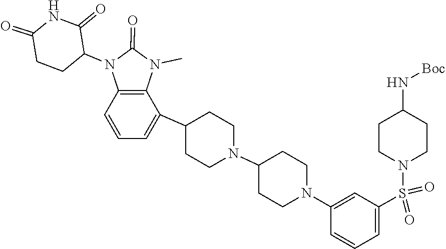 [SMILES: Cn1c(=O)n(C2CCC(=O)NC2=O)c2cccc(C3CCN(C4CCN(c5cccc(S(=O)(=O)N6CCC(NC(=O)OC(C)(C)C)CC6)c5)CC4)CC3)c21]

### Step 1: tert-Butyl (1-((3-(1,4-dioxa-8-azaspiro[4.5]decan-8-yl)phenyl)sulfonyl)piperidin-4-yl)-carbamate

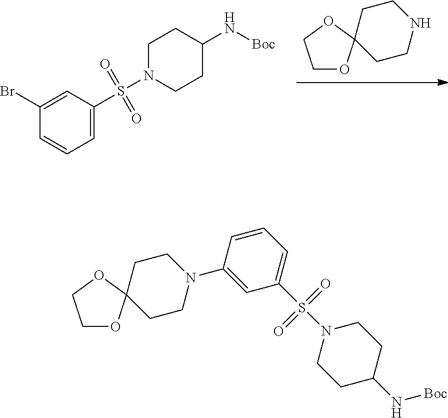 [SMILES: CC(C)(C)OC(=O)NC1CCN(S(=O)(=O)c2cccc(Br)c2)CC1 ; C1CC2(CCN1)OCCO2 ; CC(C)(C)OC(=O)NC1CCN(S(=O)(=O)c2cccc(N3CCC4(CC3)OCCO4)c2)CC1]

A mixture of tert-butyl (1-((3-bromophenyl)sulfonyl)piperidin-4-yl)carbamate (1.00 g, 2.40 mmol, 1.00 eq.), K₂CO₃ (1.16 g, 8.40 mmol, 3.50 eq.), CuI (91 mg, 0.480 mmol, 0.20 eq.), L-proline (83 mg, 0.72 mmol, 0.30 eq.) and 1,4-dioxa-8-azaspiro[4.5]decane (412 mg, 2.88 mmol, 1.20 eq.) in DMSO (10.0 mL) was stirred at 90° C. overnight. The reaction mixture was diluted with water and extracted with DCM. The combined organic layer was washed with brine, dried over anhydrous Na₂SO₄, and concentrated. The residue was purified by silica gel column chromatography eluting with PE/EtOAc (1:1) to give the title compound as yellow solid.

### Step 2: tert-Butyl (1-((3-(4-oxopiperidin-1-yl)phenyl)sulfonyl)piperidin-4-yl)carbamate

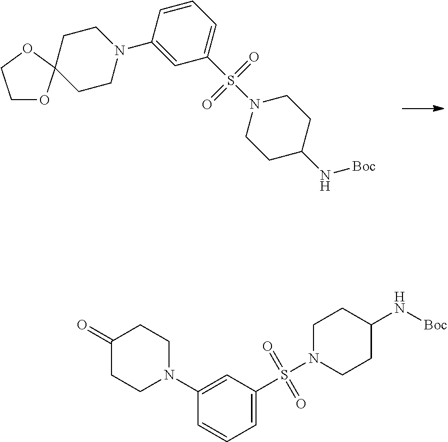 [SMILES: CC(C)(C)OC(=O)NC1CCN(S(=O)(=O)c2cccc(N3CCC4(CC3)OCCO4)c2)CC1 ; CC(C)(C)OC(=O)NC1CCN(S(=O)(=O)c2cccc(N3CCC(=O)CC3)c2)CC1]

A mixture of tert-butyl (1-((3-(1,4-dioxa-8-azaspiro[4.5]decan-8-yl)phenyl)-sulfonyl)piperidin-4-yl)carbamate (624 mg, 1.30 mmol, 1.00 eq.), TsOH·H₂O (49 mg, 0.26 mmol, 0.20 eq.) in acetone (6.0 mL) and H₂O (12.0 mL) was stirred at 60° C. overnight. The mixture was extracted with DCM. The combined organic layer was washed with brine, dried over anhydrous Na₂SO₄, and concentrated. The residue was purified by silica gel column chromatography eluting PE/EtOAc (1:1) to give the title compound as yellow solid.

### Step 3: tert-Butyl (1-((3-(4-(1-(2,6-dioxopiperidin-3-yl)-3-methyl-2-oxo-2,3-dihydro-1H-benzo[d]imidazol-4-yl)-[1,4′-bipiperidin]-1′-yl)phenyl)sulfonyl)piperidin-4-yl)carbamate

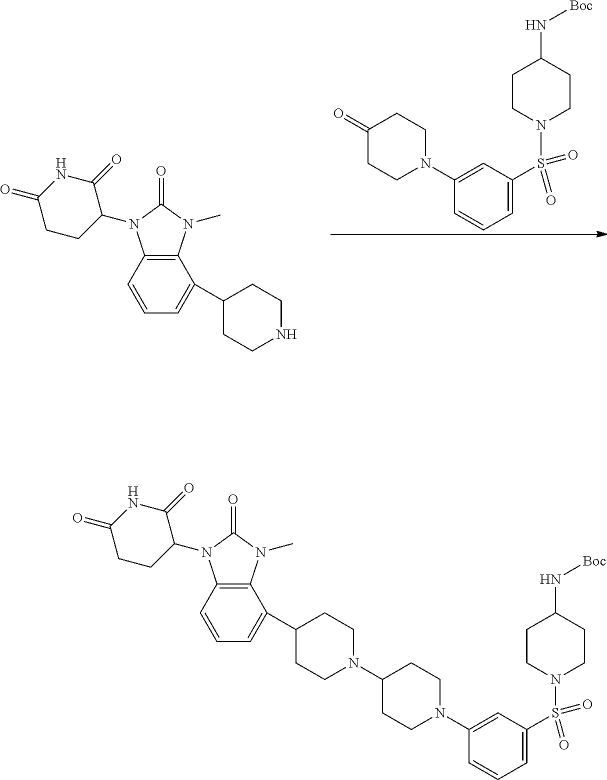 [SMILES: Cn1c(=O)n(C2CCC(=O)NC2=O)c2cccc(C3CCNCC3)c21 ; CC(C)(C)OC(=O)NC1CCN(S(=O)(=O)c2cccc(N3CCC(=O)CC3)c2)CC1 ; Cn1c(=O)n(C2CCC(=O)NC2=O)c2cccc(C3CCN(C4CCN(c5cccc(S(=O)(=O)N6CCC(NC(=O)OC(C)(C)C)CC6)c5)CC4)CC3)c21]

tert-Butyl (1-((3-(4-oxopiperidin-1-yl)phenyl)sulfonyl)piperidin-4-yl)carbamate (44 mg, 1.02 mmol, 0.90 eq.) and 1 drop of AcOH was added to a mixture of 3-(3-methyl-2-oxo-4-(piperidin-4-yl)-2,3-dihydro-1H-benzo[d]imidazol-1-yl)piperidine-2,6-dione (387 mg, 1.13 mmol, 1.00 eq.) in THF (5.0 mL). The reaction mixture was stirred at 40° C. for 0.5 h. NaBH₃CN (142 mg, 2.60 mmol, 2.00 eq.) was added at RT and stirred at RT overnight. The reaction mixture was extracted with DCM. The combined organic layer was washed with brine, dried over anhydrous Na₂SO₄, and concentrated. The residue was purified by silica gel column chromatography eluting with DCM/MeOH (10:1) to give the title compound as a yellow solid.

### Reference 34

### Synthesis of 3-(4-(azetidin-3-yl)-3-methyl-2-oxo-2,3-dihydro-1H-benzo[d]imidazol-1-yl)piperidine-2,6-dione

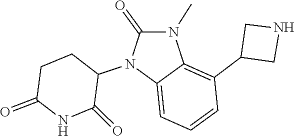 [SMILES: Cn1c(=O)n(C2CCC(=O)NC2=O)c2cccc(C3CNC3)c21]

### Step 1: tert-Butyl 3-(1-(2,6-dioxopiperidin-3-yl)-3-methyl-2-oxo-2,3-dihydro-1H-benzo[d]-imidazol-4-yl)azetidine-1-carboxylate

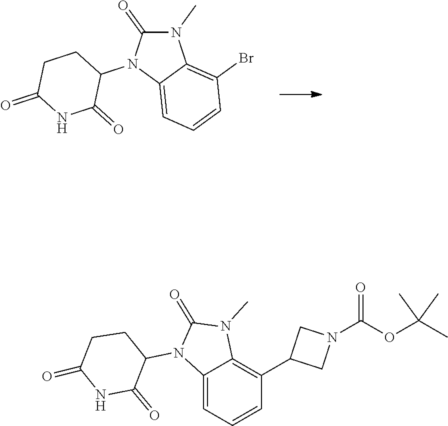 [SMILES: Cn1c(=O)n(C2CCC(=O)NC2=O)c2cccc(Br)c21 ; Cn1c(=O)n(C2CCC(=O)NC2=O)c2cccc(C3CN(C(=O)OC(C)(C)C)C3)c21]

A solution of (1-(tert-butoxycarbonyl)azetidin-3-yl)zinc (II) iodide (600 mg, 1.72 mmol, 3.00 eq.) in DMA was slowly added to a mixture of 3-(4-bromo-3-methyl-2-oxo-2,3-dihydro-1H-benzo[d]imidazol-1-yl)piperidine-2,6-dione (193 mg, 0.57 mmol, 1.00 eq.) in DMA (2.0 mL) CuI (12 mg, 0.06 mmol, 0.10 eq.) and Pd (dppf) C12 (44 mg, 0.06 mmol, 0.10 eq.). The mixture was stirred at 90° C. under N₂ overnight. The mixture was concentrated and purified by column chromatography on silica gel (EA) to the title compound as a yellow solid.

### Step 2: 3-(4-(Azetidin-3-yl)-3-methyl-2-oxo-2,3-dihydro-1H-benzo[d]imidazol-1-yl)piperidine-2,6-dione

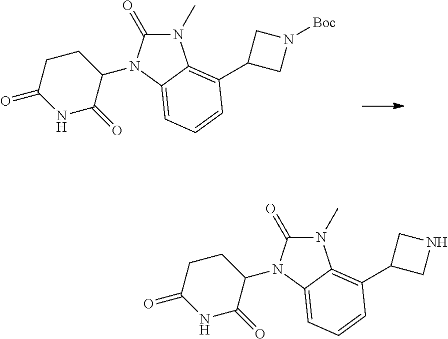 [SMILES: Cn1c(=O)n(C2CCC(=O)NC2=O)c2cccc(C3CN(C(=O)OC(C)(C)C)C3)c21 ; Cn1c(=O)n(C2CCC(=O)NC2=O)c2cccc(C3CNC3)c21]

To a solution of tert-butyl 3-(1-(2,6-dioxopiperidin-3-yl)-3-methyl-2-oxo-2,3-dihydro-1H-benzo[d]imidazol-4-yl)azetidine-1-carboxylate (23 mg, 0.055 mmol, 1.00 eq.) in DCM (1.0 mL) was added TFA (0.2 mL) dropwise and the solution was stirred at RT for 3 h. The resulting mixture was concentrated to give the crude product as a brown oil, which was used in next step without further purification.

### Reference 35

### Synthesis of tert-butyl (1-((3-((4-(2-(2,6-dioxopiperidin-3-yl)-6-fluoro-1-oxoisoindolin-5-yl)piperazin-1-yl)methyl)phenyl)sulfonyl)piperidin-4-yl)carbamate

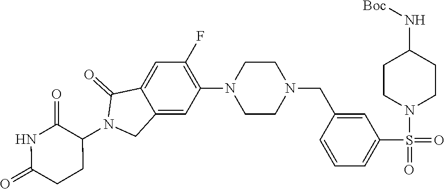 [SMILES: CC(C)(C)OC(=O)NC1CCN(S(=O)(=O)c2cccc(CN3CCN(c4cc5c(cc4F)C(=O)N(C4CCC(=O)NC4=O)C5)CC3)c2)CC1]

### Step 1: tert-Butyl 4-(5-bromo-2-fluoro-4-(methoxycarbonyl)phenyl)piperazine-1-carboxylate

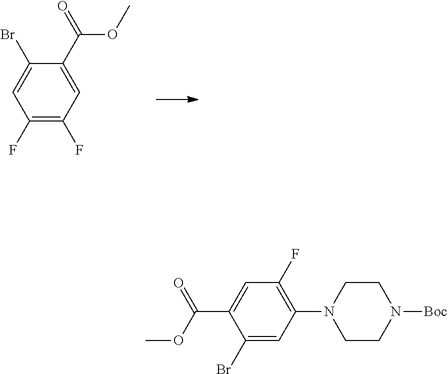 [SMILES: COC(=O)c1cc(F)c(F)cc1Br ; COC(=O)c1cc(F)c(N2CCN(C(=O)OC(C)(C)C)CC2)cc1Br]

A mixture of methyl 2-bromo-4,5-difluorobenzoate (2.00 g, 8.00 mmol, 1.00 eq.) and tert-butyl piperazine-1-carboxylate (2.23 g, 12.00 mmol, 1.50 eq.), K₂CO₃ (1.65 g, 12.00 mmol, 1.50 eq.) in DMA (6.0 mL) was stirred at 80° C. overnight. The mixture was diluted with water and extracted EA. The combined organic layer was washed with brine, dried over Na₂SO₄, concentrated. The residue was purified by flash chromatography (PE:EA=3:1) to give the title compound as a colorless oil.

### Step 2: tert-Butyl 4-(5-cyano-2-fluoro-4-(methoxycarbonyl)phenyl)piperazine-1-carboxylate

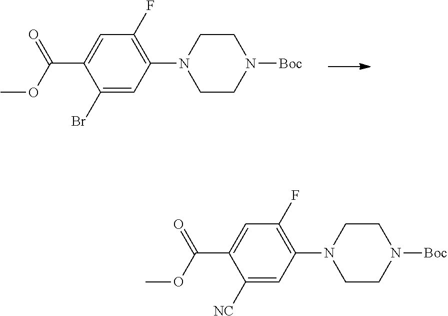 [SMILES: COC(=O)c1cc(F)c(N2CCN(C(=O)OC(C)(C)C)CC2)cc1Br ; COC(=O)c1cc(F)c(N2CCN(C(=O)OC(C)(C)C)CC2)cc1C#N]

A mixture of tert-butyl 4-(5-bromo-2-fluoro-4-(methoxycarbonyl)phenyl)-piperazine-1-carboxylate (1.50 g, 3.60 mmol, 1.00 eq.) and CuCN (484 mg, 5.40 mmol, 1.50 eq.) in DMF (6.0 mL was stirred at 100° C. overnight. The mixture was extracted with EA and washed with NH₃·H₂O. The organic layer was washed with water and brine, dried over Na₂SO₄, concentrated and purified by flash chromatography (PE:EA=3:1) to give the title compound as a white solid.

### Step 3: tert-Butyl 4-(2-(2,6-dioxopiperidin-3-yl)-6-fluoro-1-oxoisoindolin-5-yl)piperazine-1-carboxylate

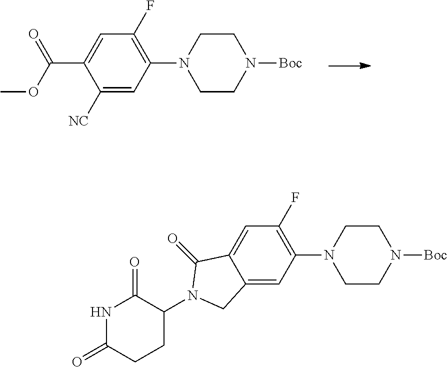 [SMILES: COC(=O)c1cc(F)c(N2CCN(C(=O)OC(C)(C)C)CC2)cc1C#N ; CC(C)(C)OC(=O)N1CCN(c2cc3c(cc2F)C(=O)N(C2CCC(=O)NC2=O)C3)CC1]

tert-Butyl 4-(5-cyano-2-fluoro-4-(methoxycarbonyl)phenyl)piperazine-1-carboxylate was converted to the title compound by proceeding analogously as described in Reference 28, Step 6-7.

### Step 4: 3-(6-Fluoro-1-oxo-5-(piperazin-1-yl) isoindolin-2-yl)piperidine-2,6-dione

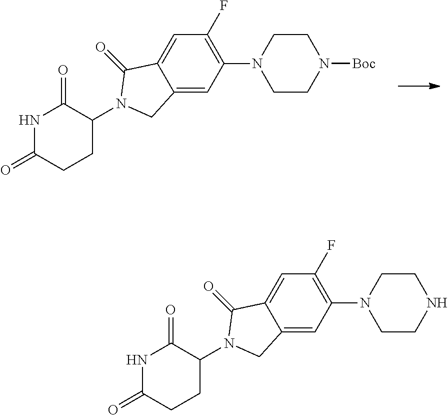 [SMILES: CC(C)(C)OC(=O)N1CCN(c2cc3c(cc2F)C(=O)N(C2CCC(=O)NC2=O)C3)CC1 ; O=C1CCC(N2Cc3cc(N4CCNCC4)c(F)cc3C2=O)C(=O)N1]

To a stirred solution of tert-butyl 4-(2-(2,6-dioxopiperidin-3-yl)-6-fluoro-1-oxoisoindolin-5-yl)piperazine-1-carboxylate (95 mg, 0.21 mmol, 1.00 eq.) in DCM (2.0 mL) was added TFA (0.5 mL) and the mixture was stirred at RT for 2 h. The reaction mixture was concentrated to give the title compound as a yellow oil.

### Step 5: tert-Butyl (1-((3-((4-(2-(2,6-dioxopiperidin-3-yl)-6-fluoro-1-oxoisoindolin-5-yl)piperazin-1-yl)methyl)phenyl)sulfonyl)piperidin-4-yl)carbamate

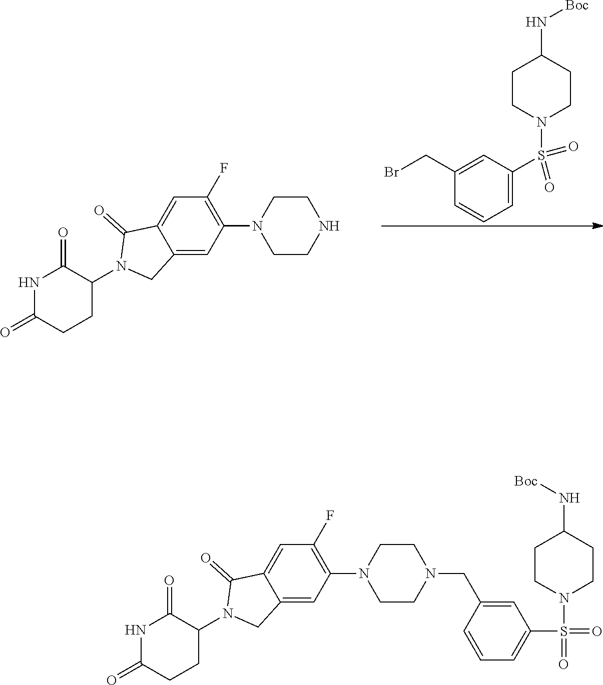 [SMILES: O=C1CCC(N2Cc3cc(N4CCNCC4)c(F)cc3C2=O)C(=O)N1 ; CC(C)(C)OC(=O)NC1CCN(S(=O)(=O)c2cccc(CBr)c2)CC1 ; CC(C)(C)OC(=O)NC1CCN(S(=O)(=O)c2cccc(CN3CCN(c4cc5c(cc4F)C(=O)N(C4CCC(=O)NC4=O)C5)CC3)c2)CC1]

A mixture of 3-(6-fluoro-1-oxo-5-(piperazin-1-yl) isoindolin-2-yl)piperidine-2,6-dione (74 mg, 0.33 mmol, 1.00 eq.), tert-butyl (1-((3-(bromomethyl)phenyl)sulfonyl)piperidin-4-yl)carbamate (138 mg, 0.32 mmol, 1.50 eq.), TEA (127 mg, 1.26 mmol, 6.00 eq.) in THF (5.0 mL) was stirred at 55° C. overnight. The mixture was diluted with water and extracted with DCM. The organic layer was washed with brine, dried over Na₂SO₄, concentrated, and the residue was purified by flash chromatography (DCM:MeOH=20:1) to give the title compound as a yellow solid.

### Reference 36

### Synthesis of tert-butyl (1-((3-((4-(2-(2,6-dioxopiperidin-3-yl)-6-fluoro-1,3-dioxoisoindolin-5-yl)piperazin-1-yl)methyl)phenyl)sulfonyl)piperidin-4-yl)carbamate

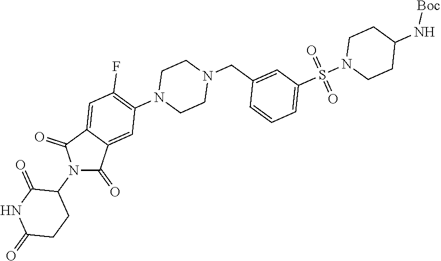 [SMILES: CC(C)(C)OC(=O)NC1CCN(S(=O)(=O)c2cccc(CN3CCN(c4cc5c(cc4F)C(=O)N(C4CCC(=O)NC4=O)C5=O)CC3)c2)CC1]

### Step 1: tert-Butyl 4-(2-(2,6-dioxopiperidin-3-yl)-6-fluoro-1,3-dioxoisoindolin-5-yl)piperazine-1-carboxylate

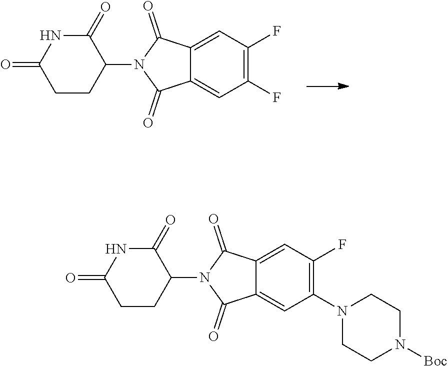 [SMILES: O=C1CCC(N2C(=O)c3cc(F)c(F)cc3C2=O)C(=O)N1 ; CC(C)(C)OC(=O)N1CCN(c2cc3c(cc2F)C(=O)N(C2CCC(=O)NC2=O)C3=O)CC1]

A mixture of tert-butyl piperazine-1-carboxylate (950 mg, 5.10 mmol, 1.00 eq.) and 2-(2,6-dioxopiperidin-3-yl)-5,6-difluoroisoindoline-1,3-dione (1.50 g, 5.10 mmol, 1.00 eq.), DIEA (1.97 g, 15.30 mmol, 3.00 eq.) in NMP (15.0 mL) was stirred at 110° C. overnight. The mixture was diluted with water and extracted EA. The organic layer was washed with brine, dried over Na₂SO₄, concentrated and the residue was purified by flash chromatography (PE:EA=1:2) to give the title compound as a yellow solid.

### Step 2: 2-(2,6-Dioxopiperidin-3-yl)-5-fluoro-6-(piperazin-1-yl)isoindoline-1,3-dione

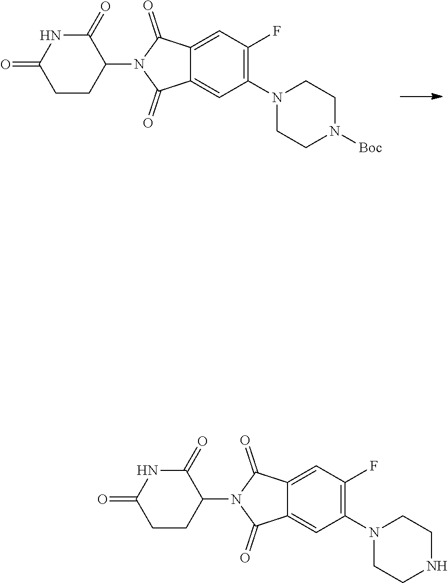 [SMILES: CC(C)(C)OC(=O)N1CCN(c2cc3c(cc2F)C(=O)N(C2CCC(=O)NC2=O)C3=O)CC1 ; O=C1CCC(N2C(=O)c3cc(F)c(N4CCNCC4)cc3C2=O)C(=O)N1]

To a stirred solution of tert-butyl 4-(2-(2,6-dioxopiperidin-3-yl)-6-fluoro-1,3-dioxoisoindolin-5-yl)piperazine-1-carboxylate (800 mg, 1.74 mmol, 1.00 eq.) in DCM (4.0 mL) was added TFA (1.0 mL) and the mixture was stirred at RT for 2 h. The reaction mixture was concentrated to give the title compound as a yellow oil.

### Step 3: 3-(Bromomethyl)benzenesulfonyl chloride

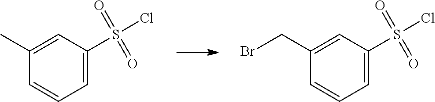

A mixture of 3-methylbenzenesulfonyl chloride (8.00 g, 41.96 mol, 1.00 eq.) NBS (8.22 g, 46.16 mol, 1.10 eq.) and benzoyl peroxide (1.46 g, 4.20 mol, 0.01 eq.) in CCl₄ (80.00 mL) was stirred at 80° C. for 12 h. The solution was filtered and the filtrate was concentrated to give crude product as white oil.

### Step 4: tert-Butyl (1-((3-(bromomethyl)phenyl)sulfonyl)piperidin-4-yl)carbamate

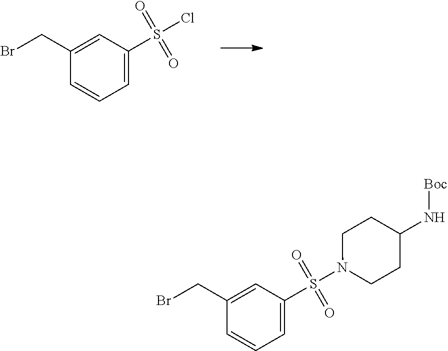 [SMILES: O=S(=O)(Cl)c1cccc(CBr)c1 ; CC(C)(C)OC(=O)NC1CCN(S(=O)(=O)c2cccc(CBr)c2)CC1]

tert-Butyl piperidin-4-ylcarbamate (5.64 g, 21.05 mol, 1.00 eq.) in THF (20.00 mL) was added to a stirred solution of 3-(bromomethyl)benzenesulfonyl chloride (3.79 g, 18.95 mol, 0.90 eq.) in THF (40.00 mL) and TEA (4.25 g, 42.10 mmol, 2.00 eq.) at 0° C. The resulting mixture was stirred at RT for 12 h, quenched with H₂O and then extracted with DCM. The combined organic layer was washed with water, dried over anhydrous Na₂SO₄, filtered, and concentrated. The residue was purified by silica gel column chromatography, eluted with PE/EA (3:1), to afford the title compound as white solid.

### Step 5: tert-Butyl (1-((3-((4-(2-(2,6-dioxopiperidin-3-yl)-6-fluoro-1,3-dioxoisoindolin-5-yl)piperazin-1-yl)methyl)phenyl)sulfonyl)piperidin-4-yl)carbamate

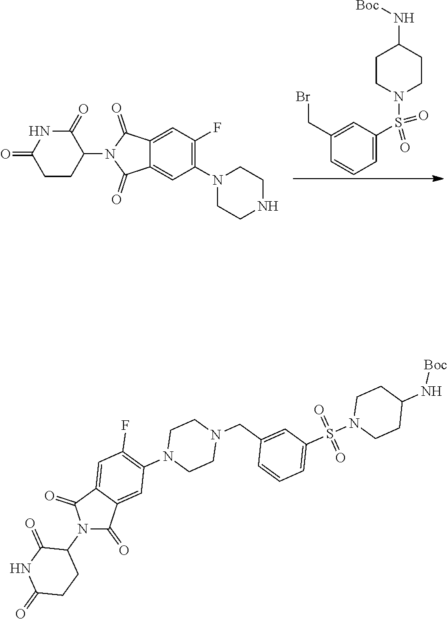 [SMILES: O=C1CCC(N2C(=O)c3cc(F)c(N4CCNCC4)cc3C2=O)C(=O)N1 ; CC(C)(C)OC(=O)NC1CCN(S(=O)(=O)c2cccc(CBr)c2)CC1 ; CC(C)(C)OC(=O)NC1CCN(S(=O)(=O)c2cccc(CN3CCN(c4cc5c(cc4F)C(=O)N(C4CCC(=O)NC4=O)C5=O)CC3)c2)CC1]

A mixture of 2-(2,6-dioxopiperidin-3-yl)-5-fluoro-6-(piperazin-1-yl)isoindoline-1,3-dione (509 mg, 1.41 mmol, 1.00 eq.), tert-butyl (1-((3-(bromomethyl)phenyl)sulfonyl)-piperidin-4-yl)carbamate (916 mg, 2.12 mmol, 1.50 eq.) TEA (854 mg, 8.46 mmol, 6.00 eq.) in THF (10.0 mL) was stirred at 55° C. overnight. The mixture was extracted DCM and water. The organic layer was washed with brine, dried over Na₂SO₄, filtered and concentrated. The residue was purified by flash chromatography (DCM:MeOH=20:1) to give the title compound as a yellow solid.

### Reference 37

### Synthesis of tert-butyl (1-((3-((8-(2-(2,6-dioxopiperidin-3-yl)-6-fluoro-1,3-dioxoisoindolin-5-yl)-3,8-diazabicyclo[3.2.1]octan-3-yl)methyl)phenyl)sulfonyl)piperidin-4-yl)carbamate

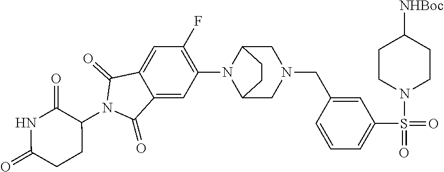 [SMILES: CC(C)(C)OC(=O)NC1CCN(S(=O)(=O)c2cccc(CN3CC4CCC(C3)N4c3cc4c(cc3F)C(=O)N(C3CCC(=O)NC3=O)C4=O)c2)CC1]

### Step 1: tert-Butyl 8-(2-(2,6-dioxopiperidin-3-yl)-6-fluoro-1,3-dioxoisoindolin-5-yl)-3,8-di-azabicyclo[3.2.1]octane-3-carboxylate

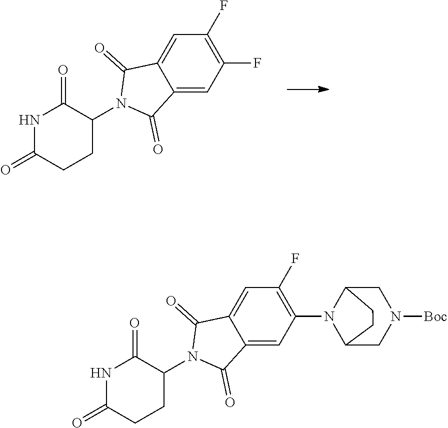 [SMILES: O=C1CCC(N2C(=O)c3cc(F)c(F)cc3C2=O)C(=O)N1 ; CC(C)(C)OC(=O)N1CC2CCC(C1)N2c1cc2c(cc1F)C(=O)N(C1CCC(=O)NC1=O)C2=O]

A mixture of 2-(2,6-dioxopiperidin-3-yl)-5,6-difluoroisoindoline-1,3-dione (200 mg, 0.68 mmol, 1.00 eq.), tert-butyl 3,8-diazabicyclo[3.2.1]octane-3-carboxylate (144 mg, 0.68 mmol, 1.00 eq.) and DIEA (263 mg, 2.04 mmol, 3.00 eq.) in NMP (3.0 mL) was stirred at 110° C. overnight. The reaction mixture was quenched with H₂O and then extracted with DCM. The combined organic layer was washed with water, dried over anhydrous Na₂SO₄, filtered, and concentrated. The residue was purified by silica gel column chromatography eluting with PE/EtOAc (1:1) to give title compound as yellow solid.

### Step 2: tert-Butyl (1-((3-((8-(2-(2,6-dioxopiperidin-3-yl)-6-fluoro-1,3-dioxoisoindolin-5-yl)-3,8-diazabicyclo[3.2.1]octan-3-yl)methyl)phenyl)sulfonyl)piperidin-4-yl)carbamate

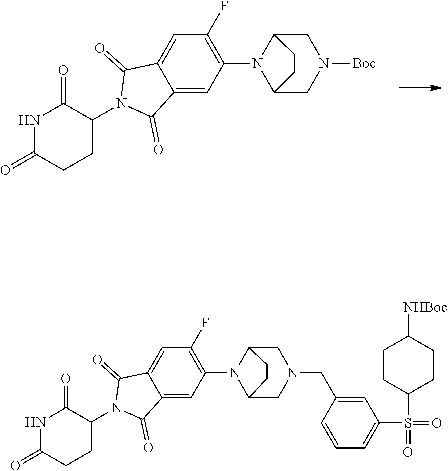 [SMILES: CC(C)(C)OC(=O)N1CC2CCC(C1)N2c1cc2c(cc1F)C(=O)N(C1CCC(=O)NC1=O)C2=O ; CC(C)(C)OC(=O)NC1CCC(S(=O)(=O)c2cccc(CN3CC4CCC(C3)N4c3cc4c(cc3F)C(=O)N(C3CCC(=O)NC3=O)C4=O)c2)CC1]

tert-Butyl 8-(2-(2,6-Dioxopiperidin-3-yl)-6-fluoro-1,3-dioxoisoindolin-5-yl)-3,8-diazabicyclo[3.2.1]octane-3-carboxylate was converted to the title compound proceeding analogously as described in Reference 36, Step 2-5.

### Reference 38

### Synthesis of tert-butyl (1-((3-((4-(2-(2,6-dioxopiperidin-3-yl)-1-oxoisoindolin-5-yl)piperazin-1-yl)-methyl)phenyl)sulfonyl)piperidin-4-yl)carbamate

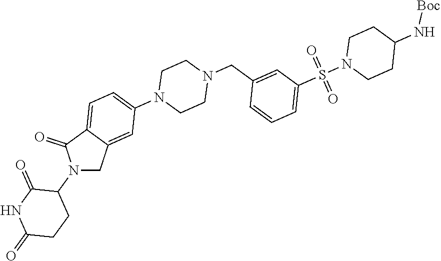 [SMILES: CC(C)(C)OC(=O)NC1CCN(S(=O)(=O)c2cccc(CN3CCN(c4ccc5c(c4)CN(C4CCC(=O)NC4=O)C5=O)CC3)c2)CC1]

### Step 1: tert-Butyl 4-(3-cyano-4-(methoxycarbonyl)phenyl)piperazine-1-carboxylate

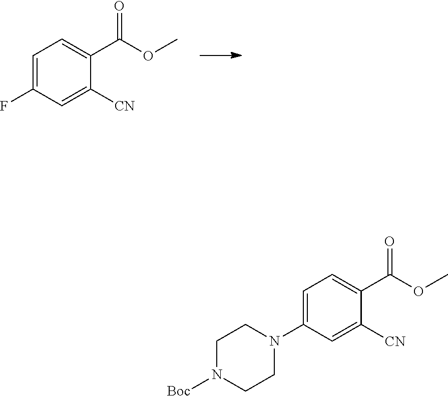 [SMILES: COC(=O)c1ccc(F)cc1C#N ; COC(=O)c1ccc(N2CCN(C(=O)OC(C)(C)C)CC2)cc1C#N]

To a stirred solution of methyl 2-cyano-4-fluorobenzoate (10.00 g, 55.80 mmol, 1.00 eq.) in DMSO (150.0 mL) was added tert-butyl piperazine-1-carboxylate (11.40 g, 61.38 mmol, 1.10 eq.) and DIEA (34.70 g, 268.96 mmol, 4.80 eq.). The resulting mixture was stirred at 110° C. for 12 h. The mixture was extracted with EtOAc washed with brine, concentrated and purified by silica gel column chromatography eluting with PE/EtOAc (3:1) to give the title compound as yellow solid.

### Step 2: tert-Butyl 4-(3-formyl-4-(methoxycarbonyl)phenyl)piperazine-1-carboxylate

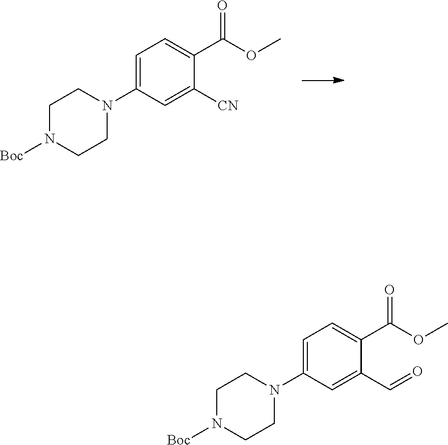 [SMILES: COC(=O)c1ccc(N2CCN(C(=O)OC(C)(C)C)CC2)cc1C#N ; COC(=O)c1ccc(N2CCN(C(=O)OC(C)(C)C)CC2)cc1C=O]

A mixture of tert-butyl 4-(3-cyano-4-(methoxycarbonyl)phenyl)piperazine-1-carboxylate (8.00 g, 23.20 mmol, 1.00 eq.) NaH₂PO₂·H₂O (5.20 g, 48.70 mmol, 2.10 eq.) and Raney-Ni (5.10 g) in pyridine:H₂O:AcOH=2:1:1 (80.0 mL) was stirred at 70° C. for 12 h. The mixture was adjusted pH=7˜8 with aq. NaHCO₃, filtered, and extracted with EtOAc. The organic layer was washed with brine, concentrated and the residue was purified by silica gel column chromatography eluting with PE/EtOAc (3:1) to give the title compound as yellow solid.

### Step 3: tert-Butyl 4-(2-(2,6-dioxopiperidin-3-yl)-1-oxoisoindolin-5-yl)piperazine-1-carboxylate

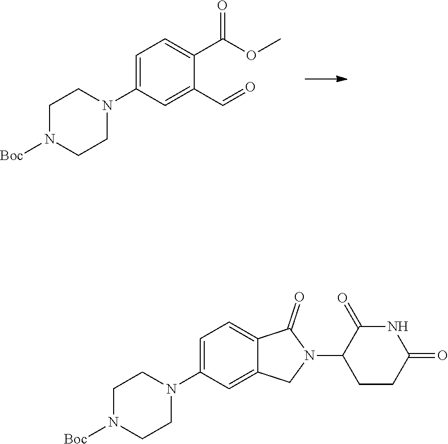 [SMILES: COC(=O)c1ccc(N2CCN(C(=O)OC(C)(C)C)CC2)cc1C=O ; CC(C)(C)OC(=O)N1CCN(c2ccc3c(c2)CN(C2CCC(=O)NC2=O)C3=O)CC1]

A mixture of 3-aminopiperidine-2,6-dione hydrochloride (2.60 g, 15.50 mmol, 1.20 eq.) DIEA (4.03 g, 31.22 mmol, 2.42 eq.), AcOH (10.63 g, 188.76 mmol, 13.78 eq.) and tert-butyl 4-(3-formyl-4-(methoxycarbonyl)phenyl)piperazine-1-carboxylate (4.50 g, 12.90 mmol, 1.00 eq.) in DCM (50.0 mL) was stirred at 35° C. for 4 h and then NaBH(OAc)₃ (8.20 g, 38.70 mmol, 3.00 eq.) was added at RT. The mixture was stirred at 40° C. for 12 h and diluted with water and extracted with EtOAc. The organic layer was washed with brine, concentrated, and the residue was purified by silica gel column chromatography eluting with PE/EtOAc (1:2) to give the title compound as white solid.

### Step 4: 3-(1-Oxo-5-(piperazin-1-yl) isoindolin-2-yl)piperidine-2,6-dione

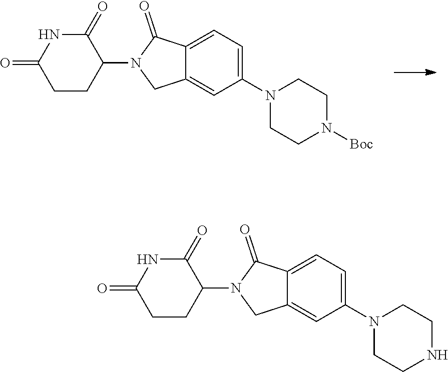 [SMILES: CC(C)(C)OC(=O)N1CCN(c2ccc3c(c2)CN(C2CCC(=O)NC2=O)C3=O)CC1 ; O=C1CCC(N2Cc3cc(N4CCNCC4)ccc3C2=O)C(=O)N1]

To a solution of tert-butyl 4-(2-(2,6-dioxopiperidin-3-yl)-1-oxoisoindolin-5-yl)piperazine-1-carboxylate (72 mg, 0.17 mmol, 1.00 eq.) in DCM (4.0 mL) was added TFA (1.0 mL). The resulting mixture was stirred at RT for 2 h and then concentrated to give the title compound as yellow oil.

### Step 5: tert-Butyl (1-((3-((4-(2-(2,6-dioxopiperidin-3-yl)-1-oxoisoindolin-5-yl)piperazin-1-yl)-methyl)phenyl)sulfonyl)piperidin-4-yl)carbamate

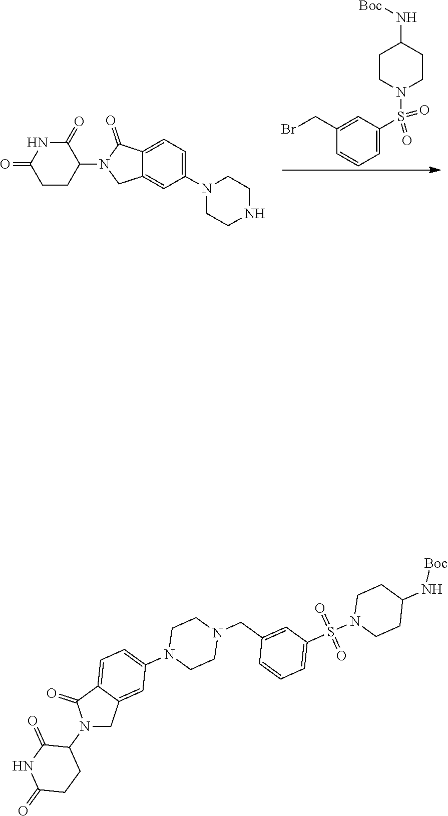 [SMILES: O=C1CCC(N2Cc3cc(N4CCNCC4)ccc3C2=O)C(=O)N1 ; CC(C)(C)OC(=O)NC1CCN(S(=O)(=O)c2cccc(CBr)c2)CC1 ; CC(C)(C)OC(=O)NC1CCN(S(=O)(=O)c2cccc(CN3CCN(c4ccc5c(c4)CN(C4CCC(=O)NC4=O)C5=O)CC3)c2)CC1]

To a stirred solution of 3-(1-oxo-5-(piperazin-1-yl) isoindolin-2-yl)piperidine-2,6-dione (55 mg, 0.17 mmol, 1.00 eq.) in THF (2.0 mL) were added TEA (52 mg, 0.51 mmol, 3.00 eq.) and tert-butyl (1-((3-(bromomethyl)phenyl)sulfonyl)piperidin-4-yl)carbamate (95 mg, 0.22 mmol, 1.30 eq.). The reaction mixture was stirred at 55° C. overnight. The reaction mixture was concentrated and purified by silica gel column chromatography eluting with DCM/MeOH (20:1) to give the title compound as a yellow solid.

### Reference 39

### Synthesis of tert-butyl (1-((3-((7-(2-(2,6-dioxopiperidin-3-yl)-1-oxoisoindolin-5-yl)-2,7-diazaspiro-[3.5]nonan-2-yl)methyl)phenyl)sulfonyl)piperidin-4-yl)carbamate

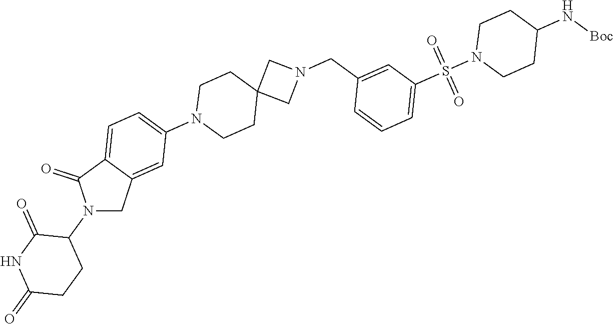 [SMILES: CC(C)(C)OC(=O)NC1CCN(S(=O)(=O)c2cccc(CN3CC4(CCN(c5ccc6c(c5)CN(C5CCC(=O)NC5=O)C6=O)CC4)C3)c2)CC1]

### Step 1: tert-Butyl 7-(3-cyano-4-(methoxycarbonyl)phenyl)-2,7-diazaspiro[3.5]nonane-2-carboxylate

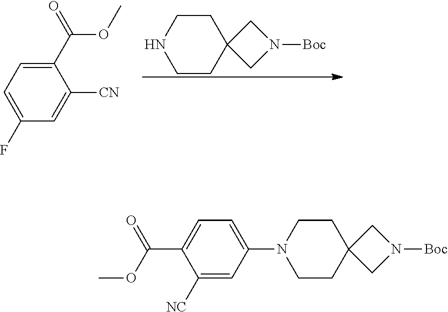 [SMILES: COC(=O)c1ccc(F)cc1C#N ; CC(C)(C)OC(=O)N1CC2(CCNCC2)C1 ; COC(=O)c1ccc(N2CCC3(CC2)CN(C(=O)OC(C)(C)C)C3)cc1C#N]

A mixture of methyl 2-cyano-4-fluorobenzoate (1.00 g, 5.58 mmol, 1.00 eq.) and tert-butyl 2,7-diazaspiro[3.5]nonane-2-carboxylate (1.39 g, 6.14 mmol, 1.10 eq.) DIEA (719 mg, 16.74 mmol, 3.00 eq.) in DMSO (10.0 mL) was stirred at 110° C. overnight. The mixture was diluted with water and extracted EA. The organic layer was washed with brine, dried over Na₂SO₄, and concentrated. The residue was purified by flash chromatography (PE:EA=3:1) to give the title compound as a white solid.

### Step 2: tert-Butyl 7-(2-(2,6-dioxopiperidin-3-yl)-1-oxoisoindolin-5-yl)-2,7-diazaspiro[3.5]nonane-2-carboxylate

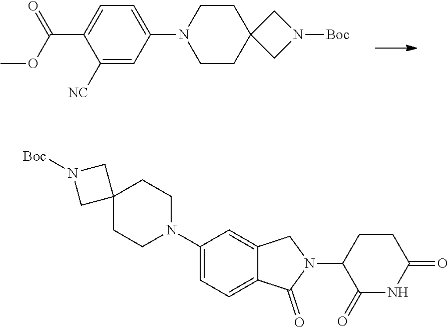 [SMILES: COC(=O)c1ccc(N2CCC3(CC2)CN(C(=O)OC(C)(C)C)C3)cc1C#N ; CC(C)(C)OC(=O)N1CC2(CCN(c3ccc4c(c3)CN(C3CCC(=O)NC3=O)C4=O)CC2)C1]

tert-butyl 7-(3-Cyano-4-(methoxycarbonyl)phenyl)-2,7-diazaspiro[3.5]nonane-2-carboxylate was converted to the title compound by proceeding analogously as described in Reference 28, Step 6-7.

### Step 3: 3-(1-Oxo-5-(2,7-diazaspiro[3.5]nonan-7-yl) isoindolin-2-yl)piperidine-2,6-dione

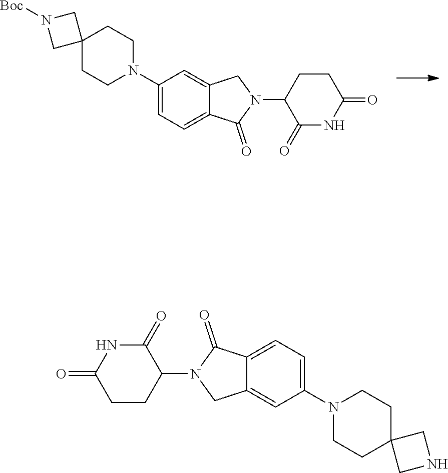 [SMILES: CC(C)(C)OC(=O)N1CC2(CCN(c3ccc4c(c3)CN(C3CCC(=O)NC3=O)C4=O)CC2)C1 ; O=C1CCC(N2Cc3cc(N4CCC5(CC4)CNC5)ccc3C2=O)C(=O)N1]

To a stirred solution of tert-butyl 7-(2-(2,6-dioxopiperidin-3-yl)-1-oxoisoindolin-5-yl)-2,7-diazaspiro[3.5]nonane-2-carboxylate (220 mg, 0.32 mmol, 1.00 eq.) in DCM (2.0 mL) was added TFA (0.5 mL) and the mixture was stirred at RT for 2 h. The reaction mixture was concentrated to give title compound as a yellow oil.

### Step 4: tert-Butyl (1-((3-((7-(2-(2,6-dioxopiperidin-3-yl)-1-oxoisoindolin-5-yl)-2,7-diazaspiro-[3.5]nonan-2-yl)methyl)phenyl)sulfonyl)piperidin-4-yl)carbamate

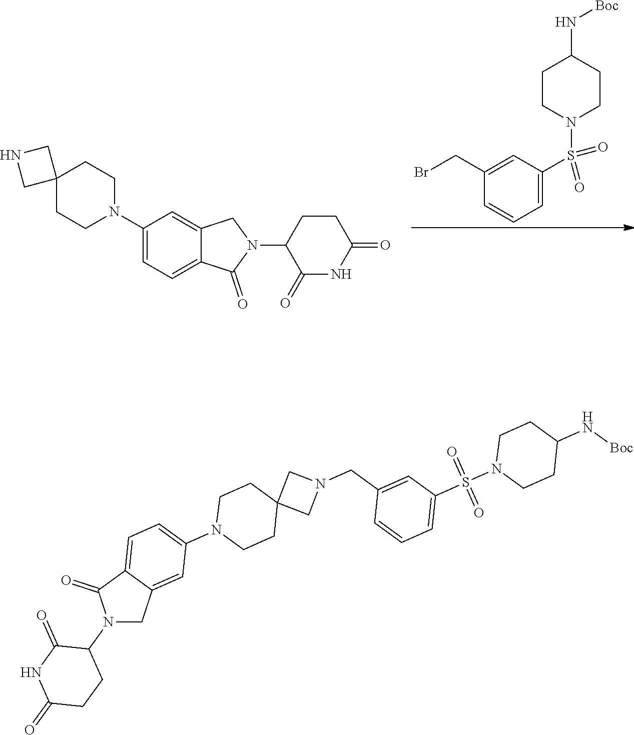 [SMILES: O=C1CCC(N2Cc3cc(N4CCC5(CC4)CNC5)ccc3C2=O)C(=O)N1 ; CC(C)(C)OC(=O)NC1CCN(S(=O)(=O)c2cccc(CBr)c2)CC1 ; CC(C)(C)OC(=O)NC1CCN(S(=O)(=O)c2cccc(CN3CC4(CCN(c5ccc6c(c5)CN(C5CCC(=O)NC5=O)C6=O)CC4)C3)c2)CC1]

A mixture of 3-(1-oxo-5-(2,7-diazaspiro[3.5]nonan-7-yl) isoindolin-2-yl)piperidine-2,6-dione (173 mg, 0.47 mmol, 1.00 eq.) and tert-butyl (1-((3-(bromomethyl)phenyl)sulfonyl)-piperidin-4-yl)carbamate (264 mg, 0.61 mmol, 1.30 eq.) TEA (285 mg, 2.82 mmol, 6.00 eq.) in THF (5.0 mL) was stirred at 55° C. overnight. The mixture was diluted with water and extracted DCM. The organic layer was washed with brine, dried over Na₂SO₄, and concentrated. The residue was purified by flash chromatography (DCM:MeOH=20:1) to give the title compound as a yellow solid.

### Reference 40

### Synthesis of rac-tert-butyl ((3R,4S)-1-((3-((4-(2-(2,6-dioxopiperidin-3-yl)-6-fluoro-1,3-dioxoisoindolin-5-yl)piperazin-1-yl)methyl)phenyl)sulfonyl)-3-fluoropiperidin-4-yl)carbamate

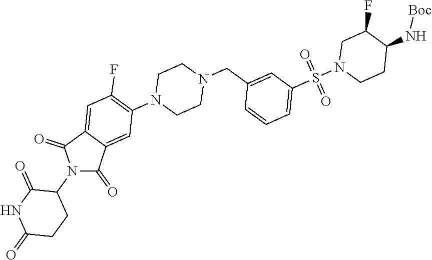 [SMILES: CC(C)(C)OC(=O)N[C@H]1CCN(S(=O)(=O)c2cccc(CN3CCN(c4cc5c(cc4F)C(=O)N(C4CCC(=O)NC4=O)C5=O)CC3)c2)C[C@H]1F]

### Step 1: rac-tert-Butyl ((3R,4S)-1-((3-(bromomethyl)phenyl)sulfonyl)-3-fluoropiperidin-4-yl)carbamate

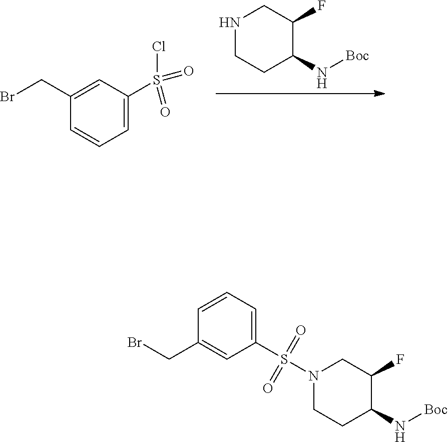 [SMILES: O=S(=O)(Cl)c1cccc(CBr)c1 ; CC(C)(C)OC(=O)N[C@H]1CCNC[C@H]1F ; CC(C)(C)OC(=O)N[C@H]1CCN(S(=O)(=O)c2cccc(CBr)c2)C[C@H]1F]

3-(Bromomethyl)-benzene-1-sulfonyl chloride (122 mg, 0.46 mmol, 1.00 eq.) in THF (1.0 mL was added to a solution of rac-tert-butyl ((3R,4S)-3-fluoropiperidin-4-yl)carbamate (100 mg, 0.46 mmol, 1.00 eq.) and TEA (93 mg, 0.92 mmol, 2.00 eq.) in THF (2.0 mL) slowly at −10° C. for 3 h. The mixture was diluted with water and extracted EA. The organic layer was washed with brine, dried over Na₂SO₄, and concentrated. The residue was purified by flash chromatography (PE:EA=4:1) to give the title compound as a white solid.

### Step 2: rac-tert-Butyl ((3R,4S)-1-((3-((4-(2-(2,6-dioxopiperidin-3-yl)-6-fluoro-1,3-dioxoisoindolin-5-yl)piperazin-1-yl)methyl)phenyl)sulfonyl)-3-fluoropiperidin-4-yl)carbamate

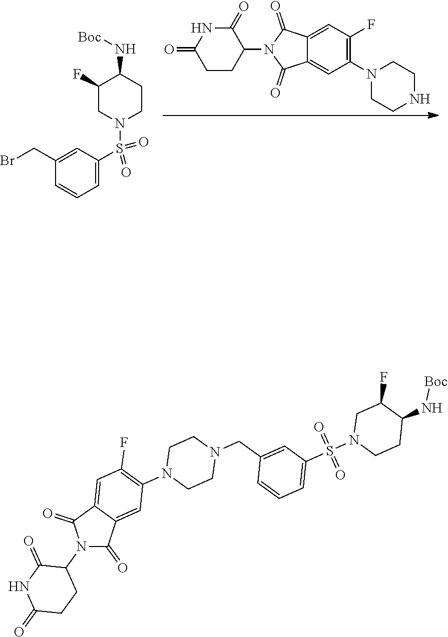 [SMILES: CC(C)(C)OC(=O)N[C@H]1CCN(S(=O)(=O)c2cccc(CBr)c2)C[C@H]1F ; O=C1CCC(N2C(=O)c3cc(F)c(N4CCNCC4)cc3C2=O)C(=O)N1 ; CC(C)(C)OC(=O)N[C@H]1CCN(S(=O)(=O)c2cccc(CN3CCN(c4cc5c(cc4F)C(=O)N(C4CCC(=O)NC4=O)C5=O)CC3)c2)C[C@H]1F]

To a solution of 2-(2,6-dioxopiperidin-3-yl)-5-fluoro-6-(piperazin-1-yl)isoindoline-1,3-dione (94 mg, 0.26 mmol, 1.00 eq.) and rac-tert-butyl ((3R,4S)-1-((3-(bromomethyl)phenyl)-sulfonyl)-3-fluoropiperidin-4-yl)carbamate (141 mg, 0.31 mmol, 1.20 eq.) in THF (4.0 mL) was added TEA (131 mg, 1.30 mmol, 5.00 eq.) and the mixture was stirred at 55° C. overnight. The mixture was diluted with water and extracted DCM. The organic layer was washed with brine, dried over Na₂SO₄, and concentrated. The residue was purified by flash chromatography (DCM:MeOH=20:1) to give the title compound as a yellow solid.

### Reference 41

### Synthesis of tert-butyl (1-((3-((1-((2-(2,6-dioxopiperidin-3-yl)-1-oxoisoindolin-5-yl)-methyl)piperidin-4-yl)oxy)phenyl)sulfonyl)piperidin-4-yl)carbamate

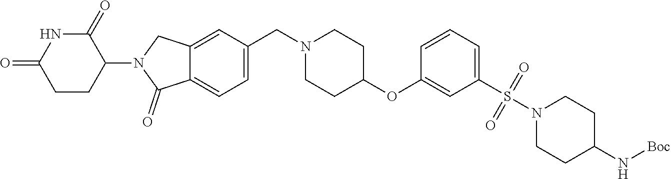 [SMILES: CC(C)(C)OC(=O)NC1CCN(S(=O)(=O)c2cccc(OC3CCN(Cc4ccc5c(c4)CN(C4CCC(=O)NC4=O)C5=O)CC3)c2)CC1]

### Step 1: 2-(2,6-Dioxopiperidin-3-yl)-1-oxoisoindoline-5-carbonitrile

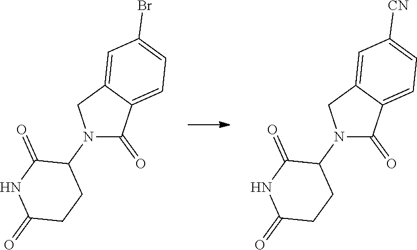 [SMILES: O=C1CCC(N2Cc3cc(Br)ccc3C2=O)C(=O)N1 ; N#Cc1ccc2c(c1)CN(C1CCC(=O)NC1=O)C2=O]

A mixture of 3-(5-bromo-1-oxoisoindolin-2-yl)piperidine-2,6-dione (2.00 g, 6.21 mmol, 1.00 eq.) and Zn(CN)₂ (438 mg, 3.73 mmol, 0.60 eq.) Pd(pph₃)₄ (714 mg) in DMF (30.0 mL) was stirred at 100° C. overnight. The mixture was extracted with DCM and purified by silica gel column chromatography eluting with PE/EtOAc (1:2) to give the title compound as yellow solid.

### Step 2: 2-(2,6-Dioxopiperidin-3-yl)-1-oxoisoindoline-5-carbaldehyde

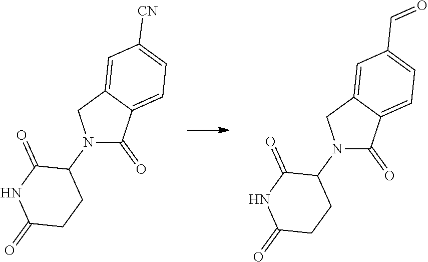 [SMILES: N#Cc1ccc2c(c1)CN(C1CCC(=O)NC1=O)C2=O ; O=Cc1ccc2c(c1)CN(C1CCC(=O)NC1=O)C2=O]

A mixture of 2-(2,6-dioxopiperidin-3-yl)-1-oxoisoindoline-5-carbonitrile (1.20 g, 4.46 mmol, 1.00 eq.), NaH₂PO₂·H₂O (993 mg, 9.37 mmol, 2.10 eq.) and Raney-Ni (500 mg) in pyridine:H₂O:AcOH (40.0 mL, 2:2:1) was stirred at 70° C. overnight. The reaction mixture was filtered and washed with aq. NaHCO₃. The organic layer was concentrated and the residue was purified by silica gel column chromatography eluting PE/EtOAc (1:2) to give the title compound as yellow solid.

### Step 3: Benzyl 4-(3-((4-((tert-butoxycarbonyl)amino)piperidin-1-yl)sulfonyl) phenoxy) piperidine-1-carboxylate

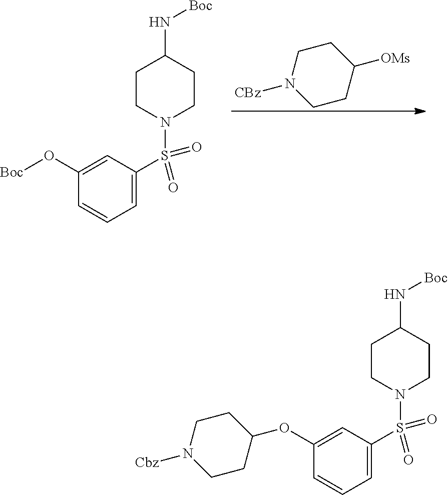

A mixture of tert-butyl (1-((3-((tert-butoxycarbonyl)oxy)phenyl)sulfonyl)piperidin-4-yl)carbamate (7.30 g, 16.0 mmol, 1.00 eq.), benzyl 4-((methylsulfonyl)oxy)piperidine-1-carboxylate (7.52 g, 24 mmol, 1.50 eq.) and Cs₂CO₃ (10.4 g, 32 mmol, 2.00 eq.) in DMSO (70.0 mL) was stirred at 90° C. for 4 h and then extracted with EtOAc. The organic layer was concentrated and the residue was purified by silica gel column chromatography eluting PE/EtOAc (3:1) to give the title compound as a yellow solid.

### Step 4: tert-Butyl (1-((3-(piperidin-4-yloxy)phenyl)sulfonyl)piperidin-4-yl)carbamate

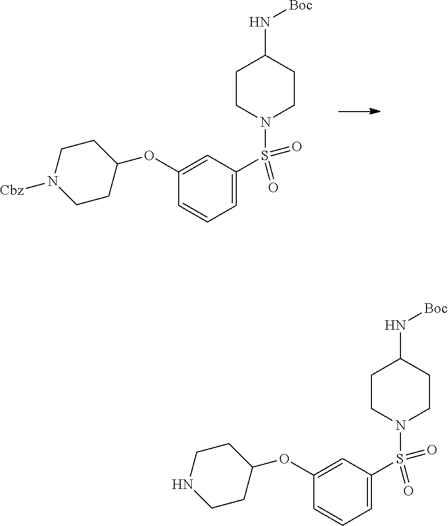 [SMILES: CC(C)(C)OC(=O)NC1CCN(S(=O)(=O)c2cccc(OC3CCN(C(=O)OCc4ccccc4)CC3)c2)CC1 ; CC(C)(C)OC(=O)NC1CCN(S(=O)(=O)c2cccc(OC3CCNCC3)c2)CC1]

A mixture of benzyl 4-(3-((4-((tert-butoxycarbonyl)amino)piperidin-1-yl)sulfonyl)-phenoxy) piperidine-1-carboxylate (6.0 g, 10.47 mmol, 1.00 eq.), HCOONH₄ (3.3 g, 52.35 mmol, 5.00 eq.), and Pd(OH)₂ (1.2 g) in EtOH (60.0 mL) was stirred at 70° C. for 4 h. The mixture was filtered and concentrated to give the title compound as a white solid.

### Step 5: tert-Butyl (1-((3-((1-((2-(2,6-dioxopiperidin-3-yl)-1-oxoisoindolin-5-yl)methyl)piperidin-4-yl)oxy)phenyl)sulfonyl)piperidin-4-yl)carbamate

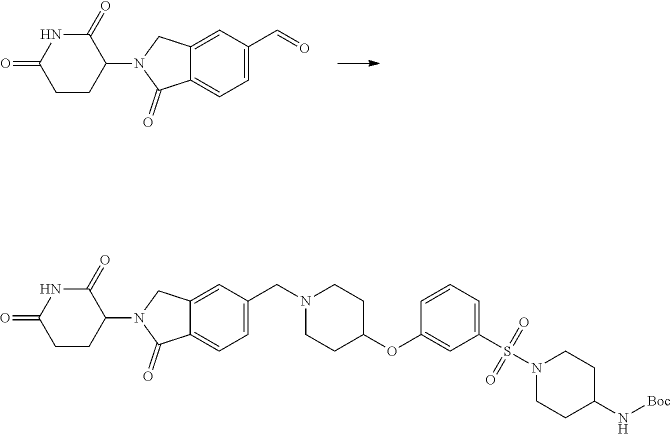 [SMILES: O=Cc1ccc2c(c1)CN(C1CCC(=O)NC1=O)C2=O ; CC(C)(C)OC(=O)NC1CCN(S(=O)(=O)c2cccc(OC3CCN(Cc4ccc5c(c4)CN(C4CCC(=O)NC4=O)C5=O)CC3)c2)CC1]

To a mixture of 2-(2,6-dioxopiperidin-3-yl)-1-oxoisoindoline-5-carbaldehyde (100 mg, 0.37 mmol, 1.00 eq.) in THF (3.0 mL) were added tert-butyl (1-((3-(piperidin-4-yloxy)phenyl)-sulfonyl)piperidin-4-yl)carbamate (169 mg, 0.39 mmol, 1.05 eq.) and 1 drop of AcOH. The mixture was stirred at 40° C. for 0.5 h. NaBH₃CN (47 mg, 0.74 mmol, 2.00 eq.) was added and stirred at RT for 16 h. The reaction mixture was diluted with water and extracted DCM. The organic layer was washed with brine, dried over Na₂SO₄, and concentrated. The residue was purified by silica gel column chromatography eluting with DCM/MeOH (20:1) to give the title compound as a yellow solid.

### Reference 42

### Synthesis of tert-butyl (1-((3-(4-(1-(2-(2,6-dioxopiperidin-3-yl)-1-oxoisoindolin-5-yl)azetidin-3-yl)piperazin-1-yl)phenyl)sulfonyl)piperidin-4-yl)carbamate

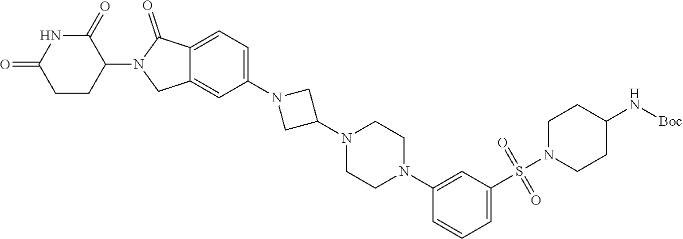 [SMILES: CC(C)(C)OC(=O)NC1CCN(S(=O)(=O)c2cccc(N3CCN(C4CN(c5ccc6c(c5)CN(C5CCC(=O)NC5=O)C6=O)C4)CC3)c2)CC1]

### Step 1: Benzyl 3-(4-(3-((4-((tert-butoxycarbonyl)amino)piperidin-1-yl)sulfonyl)phenyl)piperazin-1-yl)azetidine-1-carboxylate

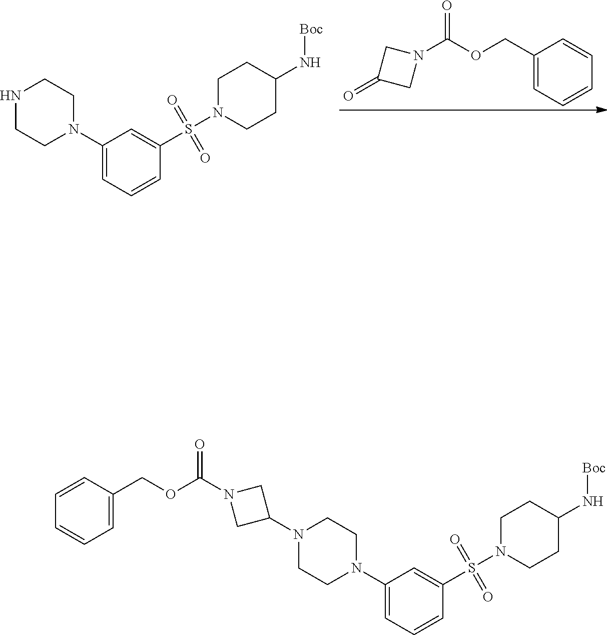 [SMILES: CC(C)(C)OC(=O)NC1CCN(S(=O)(=O)c2cccc(N3CCNCC3)c2)CC1 ; O=C1CN(C(=O)OCc2ccccc2)C1 ; CC(C)(C)OC(=O)NC1CCN(S(=O)(=O)c2cccc(N3CCN(C4CN(C(=O)OCc5ccccc5)C4)CC3)c2)CC1]

To a mixture of tert-butyl (1-((3-(piperazin-1-yl)phenyl)sulfonyl)piperidin-4-yl)carbamate (740 mg, 1.75 mmol, 1.00 eq.) in THF (10.0 mL) were added AcOH (3 drops) and benzyl 3-oxoazetidine-1-carboxylate (718 mg, 3.50 mmol, 2.00 eq.). The solution was stirred at 45° C. for 0.5 h. The solution was cooled to RT and NaBH₃CN (220 mg, 3.50 mmol, 2.00 eq.) was added. The solution was stirred at RT overnight and then extracted with EtOAc. The mixture was diluted with water and extracted DCM. The organic layer was washed with brine, dried over Na₂SO₄, and concentrated. The residue was purified by silica gel column chromatography eluting DCM/MeOH (20:1) to give the title compound as a white oil.

### Step 2: tert-Butyl (1-((3-(4-(1-(2-(2,6-dioxopiperidin-3-yl)-1-oxoisoindolin-5-yl)azetidin-3-yl)piperazin-1-yl)phenyl)sulfonyl)piperidin-4-yl)carbamate

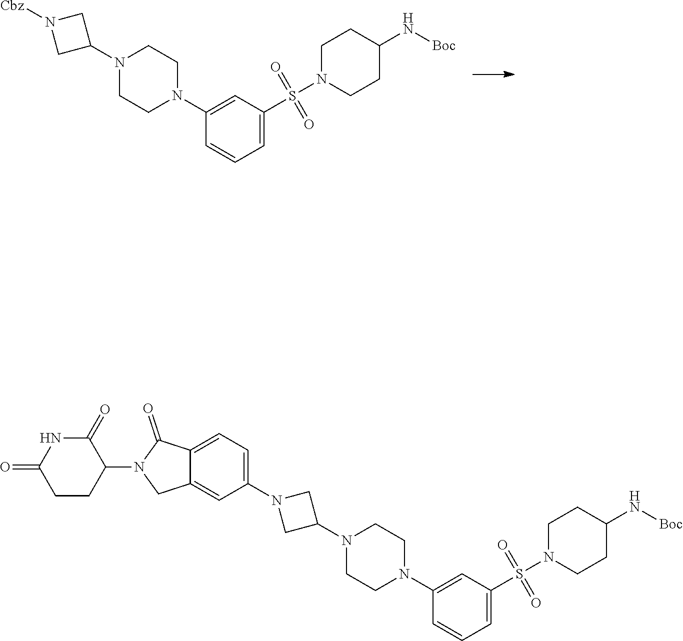 [SMILES: CC(C)(C)OC(=O)NC1CCN(S(=O)(=O)c2cccc(N3CCN(C4CN(C(=O)OCc5ccccc5)C4)CC3)c2)CC1 ; CC(C)(C)OC(=O)NC1CCN(S(=O)(=O)c2cccc(N3CCN(C4CN(c5ccc6c(c5)CN(C5CCC(=O)NC5=O)C6=O)C4)CC3)c2)CC1]

Benzyl 3-(4-(3-((4-((tert-butoxycarbonyl)amino)piperidin-1-yl)sulfonyl)phenyl)-piperazin-1-yl)azetidine-1-carboxylate was converted to the title compound using similar procedure as described in Reference 28, Step 4-7.

### Reference 43

### Synthesis of tert-butyl (1-((3-(3-(4-(2-(2,6-dioxopiperidin-3-yl)-1-oxoisoindolin-5-yl)piperazin-1-yl)-azetidin-1-yl)phenyl)sulfonyl)piperidin-4-yl)carbamate

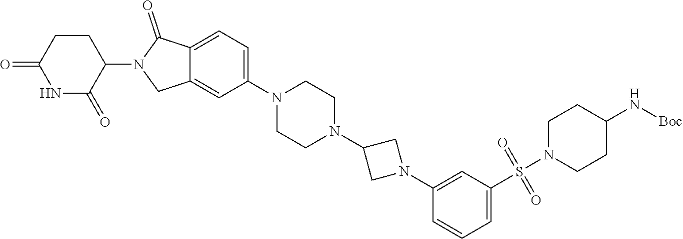 [SMILES: CC(C)(C)OC(=O)NC1CCN(S(=O)(=O)c2cccc(N3CC(N4CCN(c5ccc6c(c5)CN(C5CCC(=O)NC5=O)C6=O)CC4)C3)c2)CC1]

### Step 1: 3-Hydroxyazetidine

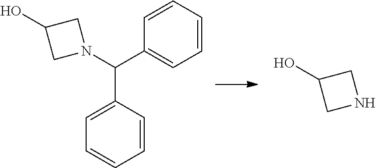 [SMILES: OC1CN(C(c2ccccc2)c2ccccc2)C1 ; OC1CNC1]

To a stirred solution of 1-benzhydrylazetidin-3-ol (5.00 g, 20.92 mmol, 1.00 eq.) and Pd(OH)₂, (3.50 g) in MeOH (130.00 mL) was added AcOH (18.50 mL). The resulting mixture was stirred at 50° C. under H₂ (50 psi) for 12 h. HCl (aq) was added to adjust the pH of the solution to pH 3. The solution was concentrated to give crude product as white oil.

### Step 2: tert-Butyl (1-((3-(3-hydroxyazetidin-1-yl)phenyl)sulfonyl)piperidin-4-yl)carbamate

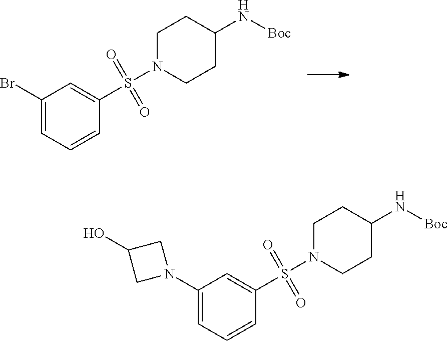 [SMILES: CC(C)(C)OC(=O)NC1CCN(S(=O)(=O)c2cccc(Br)c2)CC1 ; CC(C)(C)OC(=O)NC1CCN(S(=O)(=O)c2cccc(N3CC(O)C3)c2)CC1]

A mixture of tert-butyl (1-((3-bromophenyl)sulfonyl)piperidin-4-yl)carbamate (5.83 g, 13.95 mmol, 1.00 eq.), K₂CO₃ (6.74 g, 48.83 mmol, 3.50 eq.), CuI (0.53 g, 2.79 mmol, 0.20 eq.), L-PRO (481 mg, 4.19 mmol, 0.30 eq.) and 3-hydroxyazetidine (2.28 g, 20.92 mmol, 1.50 eq.) in DMSO (50.00 mL) was stirred at 90° C. for 12 h. The mixture was quenched with H₂O and extracted with EtOAc. The organic layer was concentrated and purified by silica gel column chromatography eluting with PE/EtOAc (2:1) to give the title compound as white solid.

### Step 3: tert-Butyl (1-((3-(3-oxoazetidin-1-yl)phenyl)sulfonyl)piperidin-4-yl)carbamate

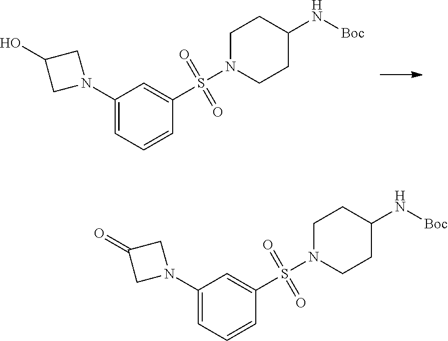 [SMILES: CC(C)(C)OC(=O)NC1CCN(S(=O)(=O)c2cccc(N3CC(O)C3)c2)CC1 ; CC(C)(C)OC(=O)NC1CCN(S(=O)(=O)c2cccc(N3CC(=O)C3)c2)CC1]

To a stirred solution of tert-butyl (1-((3-(3-hydroxyazetidin-1-yl)phenyl)sulfonyl)-piperidin-4-yl)carbamate (0.50 g, 1.22 mmol, 1.00 eq.) in DCM (5.00 mL) was added Dess-Martin (1.03 g, 2.44 mmol, 2.00 eq.) and the mixture was stirred at 0° C. for 3 h. The mixture was diluted with sodium thiosulfate (aq) and extracted with DCM. The organic layer was concentrated and the residue was purified by silica gel column chromatography, eluted with EtOAc/PE (3:1), to give the title compound as a white solid.

### Step 4: tert-Butyl (1-((3-(3-(4-(2-(2,6-dioxopiperidin-3-yl)-1-oxoisoindolin-5-yl)piperazin-1-yl)-azetidin-1-yl)phenyl)sulfonyl)piperidin-4-yl)carbamate

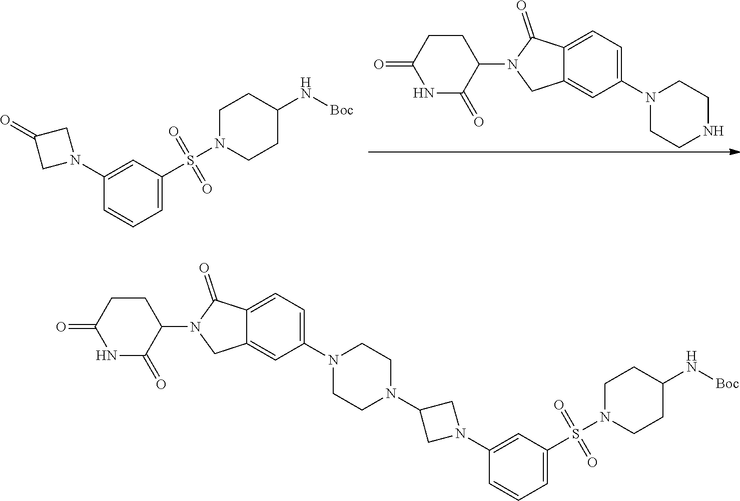 [SMILES: CC(C)(C)OC(=O)NC1CCN(S(=O)(=O)c2cccc(N3CC(=O)C3)c2)CC1 ; O=C1CCC(N2Cc3cc(N4CCNCC4)ccc3C2=O)C(=O)N1 ; CC(C)(C)OC(=O)NC1CCN(S(=O)(=O)c2cccc(N3CC(N4CCN(c5ccc6c(c5)CN(C5CCC(=O)NC5=O)C6=O)CC4)C3)c2)CC1]

To a solution of tert-butyl (1-((3-(3-oxoazetidin-1-yl)phenyl)sulfonyl)piperidin-4-yl)carbamate (40.00 mg, 0.10 mmol, 1.00 eq.) in THF (1.00 mL) and DMF (0.50 mL) were added AcOH (3 drops) and 3-(1-oxo-5-(piperazin-1-yl) isoindolin-2-yl)piperidine-2,6-dione (39.00 mg, 0.12 mmol, 1.20 eq.). The solution was stirred at 45° C. for 45 min. The solution cooled to RT and NaBH₃CN (13.00 mg, 0.20 mmol, 2.00 eq.) was added. The mixture was stirred at RT for 12 h and then diluted with water and extracted with EtOAc. The combined organic layer was washed with brine, dried over anhydrous Na₂SO₄ and concentrated. The residue was purified by TLC, eluted with DCM/MeOH (20:1), to afford the title compound as a white solid.

### Reference 44

### Synthesis of tert-butyl (1-((3-(3-(4-(1-(2,6-dioxopiperidin-3-yl)-3-methyl-2-oxo-2,3-dihydro-1H-benzo[d]imidazol-4-yl)piperidin-1-yl)azetidin-1-yl)phenyl)sulfonyl)piperidin-4-yl)carbamate

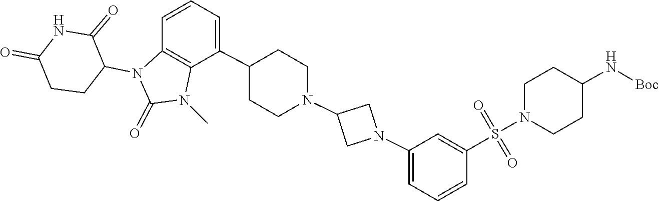 [SMILES: Cn1c(=O)n(C2CCC(=O)NC2=O)c2cccc(C3CCN(C4CN(c5cccc(S(=O)(=O)N6CCC(NC(=O)OC(C)(C)C)CC6)c5)C4)CC3)c21]

To a solution of 3-(3-methyl-2-oxo-4-(piperidin-4-yl)-2,3-dihydro-1H-benzo[d]imidazol-1-yl)piperidine-2,6-dione (41.00 mg, 0.12 mmol, 1.00 eq) in THF (2.00 mL) and DMF (0.50 mL) were added AcOH (3 drops) and tert-butyl (1-((3-(3-oxoazetidin-1-yl)phenyl)sulfonyl)piperidin-4-yl)carbamate (98.00 mg, 0.24 mmol, 2.00 eq.). The solution was stirred at 45° C. for 0.5 h. Then the solution cooled to RT and NaBH₃CN (15.08 mg, 0.24 mmol, 2.00 eq.) was added. The mixture was stirred at RT for 12 h and then diluted with water and extracted with EtOAc. The combined organic layer was washed with brine, dried over anhydrous Na₂SO₄ and concentrated. The residue was purified by TLC, eluted with DCM/MeOH (20:1), to afford the title compound as a white solid.

### Reference 45

### Synthesis of rac-1-(6-(1-(3-(((3R,4S)-4-amino-3-fluoropiperidin-1-yl)sulfonyl)benzyl)piperidin-4-yl)-1-methyl-1H-indazol-3-yl)dihydropyrimidine-2,4(1H,3H)-dione

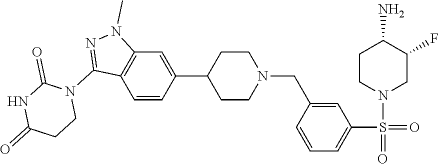 [SMILES: Cn1nc(N2CCC(=O)NC2=O)c2ccc(C3CCN(Cc4cccc(S(=O)(=O)N5CC[C@H](N)[C@H](F)C5)c4)CC3)cc21]

### Step 1: 6-Bromo-1-methyl-1H-indazol-3-amine

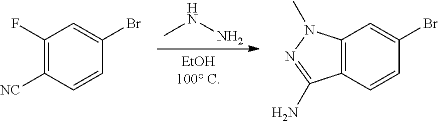

To a stirred solution of 4-bromo-2-fluorobenzonitrile (10 g, 0.05 mol, 1.00 eq.) in EtOH (50.0 mL) was added methylhydrazine (57 g, 0.50 mol, 10.00 eq.) and the mixture was stirred at 100° C. 30 h in sealed tube. Then the mixture was concentrated, and added water. The mixture was filtered to give title compound as pale yellow solid.

### Step 2: Methyl 3-((6-bromo-1-methyl-1H-indazol-3-yl)amino) propanoate

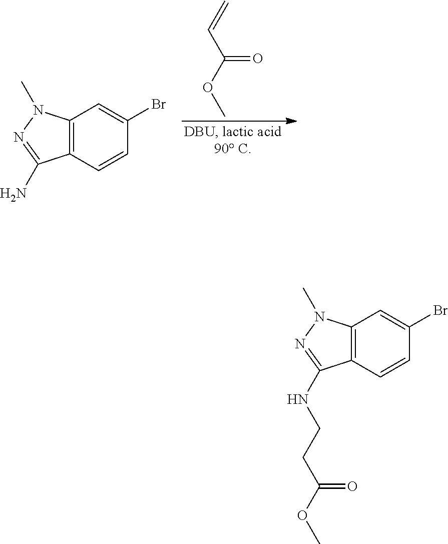

Methyl acrylate (209.00 g, 2.43 mol, 10.00 eq.) was added to a solution of 6-bromo-1-methyl-1H-indazol-3-amine (55.00 g, 0.24 mol, 1.00 eq.), DBU (55.00 g, 0.36 mol, 1.50 eq.), lactic acid (33.00 g, 0.36 mol, 1.50 eq.) at 0° C., and the mixture was stirred at 90° C. 20 h under N₂. The mixture was purified by column chromatography on silica gel (EA:PE=0 to 100%) to give the title compound as yellow solid.

### Step 3: Methyl 3-(1-(6-bromo-1-methyl-1H-indazol-3-yl) ureido) propanoate

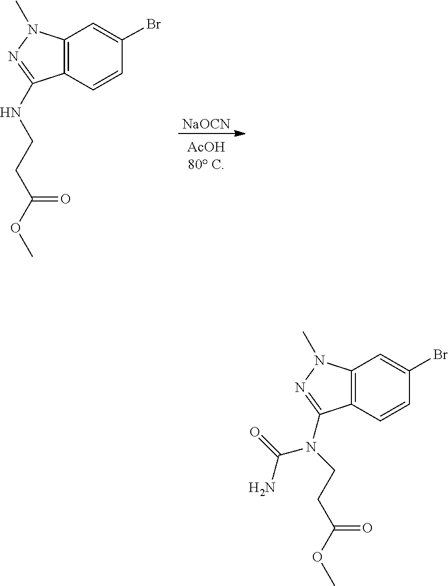

NaOCN (26.00 g, 0.32 mol, 2.00 eq.) was added to a solution of methyl 3-((6-bromo-1-methyl-1H-indazol-3-yl)amino) propanoate (50.00 g, 0.16 mol, 1.00 eq.) in AcOH (500.0 mL), and the mixture was stirred at 80° C. 20 h under N₂. The mixture was diluted with water and extracted with EA, and the organic layer was washed with sat. NaHCO₃ aq., water, brine, dried over Na₂SO₄, concentrated to give the title compound as yellow solid.

### Step 4: 1-(6-Bromo-1-methyl-1H-indazol-3-yl)dihydropyrimidine-2,4(1H,3H)-dione

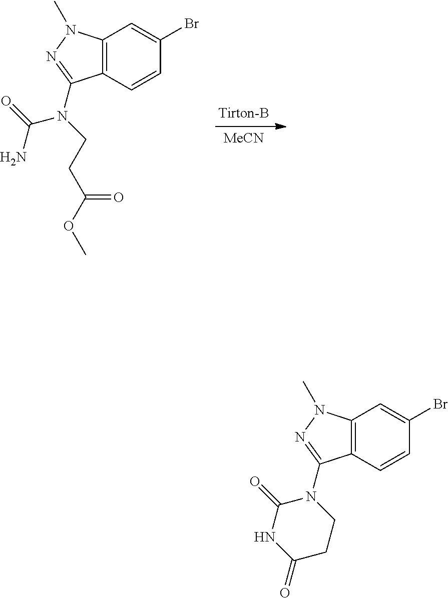 [SMILES: "MeCN" ; COC(=O)CCN(C(N)=O)c1nn(C)c2cc(Br)ccc12 ; Cn1nc(N2CCC(=O)NC2=O)c2ccc(Br)cc21]

To a solution of methyl 3-(1-(6-bromo-1-methyl-1H-indazol-3-yl) ureido) propanoate (56.00 g, 0.16 mol, 1.00 eq.) in MeCN (500.0 mL) was added Tirton-B (7.90 g, 0.05 mol, 0.30 eq.) and stirred at r.t. for 20 h under N₂. The mixture was concentrated, then diluted with water. The mixture was filtered and solid was washed with water, air dried to give the title compound as pale yellow solid.

### Step 5: tert-Butyl 4-(3-(2,4-dioxotetrahydropyrimidin-1(2H)-yl)-1-methyl-1H-indazol-6-yl)-5,6-dihydropyridine-1(2H)-carboxylate

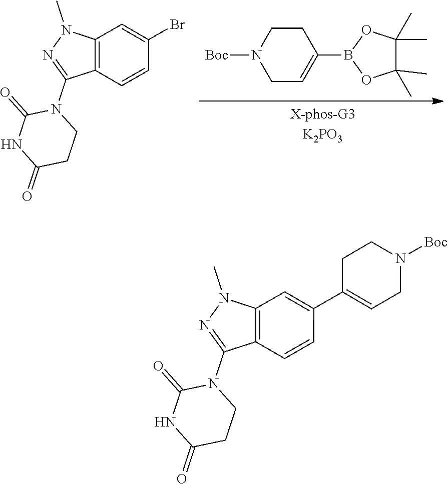 [SMILES: Cn1nc(N2CCC(=O)NC2=O)c2ccc(Br)cc21 ; CC(C)(C)OC(=O)N1CC=C(B2OC(C)(C)C(C)(C)O2)CC1 ; Cn1nc(N2CCC(=O)NC2=O)c2ccc(C3=CCN(C(=O)OC(C)(C)C)CC3)cc21]

To a mixture of 1-(6-bromo-1-methyl-1H-indazol-3-yl)dihydropyrimidine-2,4(1H,3H)-dione (1.10 g, 3.41 mmol, 1.00 eq.) in 1,4-dioxane/H₂O (10 mL/1 mL) was added tert-butyl 4-(4,4,5,5-tetramethyl-1,3,2-dioxaborolan-2-yl)-5,6-dihydropyridine-1(2H)-carboxylate (1.60 g, 5.11 mmol, 1.50 eq.), K₃PO₄ (2.20 g, 10.22 mmol, 3.00 eq.) and X-Phos-Pd G3 (289 mg, 0.34 mmol, 0.10 eq.), and the mixture was stirred at 60° C. under N₂ for 3 h. The mixture was diluted with DCM, and the organic layer was washed with water and brine, dried over Na₂SO₄, concentrated and purified by column chromatography on silica gel (DCM:MeOH=20:1) to give the title compound as yellow solid.

### Step 6: tert-Butyl 4-(3-(2,4-dioxotetrahydropyrimidin-1(2H)-yl)-1-methyl-1H-indazol-6-yl)piperidine-1-carboxylate

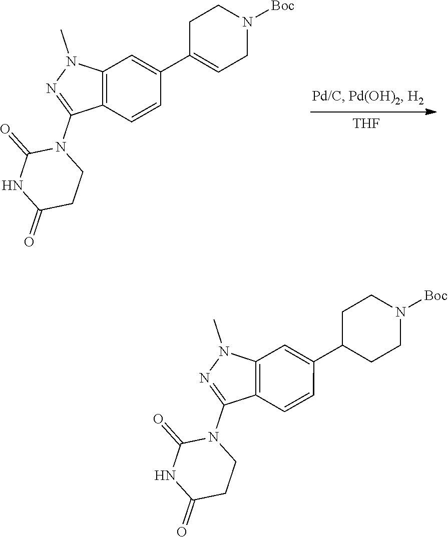 [SMILES: Cn1nc(N2CCC(=O)NC2=O)c2ccc(C3=CCN(C(=O)OC(C)(C)C)CC3)cc21 ; Cn1nc(N2CCC(=O)NC2=O)c2ccc(C3CCN(C(=O)OC(C)(C)C)CC3)cc21 ; "THF"]

A mixture of tert-butyl 4-(3-(2,4-dioxotetrahydropyrimidin-1(2H)-yl)-1-methyl-1H-indazol-6-yl)-5,6-dihydropyridine-1(2H)-carboxylate (300 mg, 0.71 mmol, 1.00 eq.), Pd/C (150 mg, 50% wt) and Pd(OH)₂ (150 mg, 50% wt) in THF (20.0 mL) was stirred under H₂ at 50° C. and 50 psi overnight. The mixture was filtered and the filtrate was concentrated and purified by column chromatography on silica gel (PE:EA=1:1) to give the title compound as yellow solid.

### Step 7: 1-(1-Methyl-6-(piperidin-4-yl)-1H-indazol-3-yl)dihydropyrimidine-2,4(1H,3H)-dione 2,2,2-trifluoroacetate

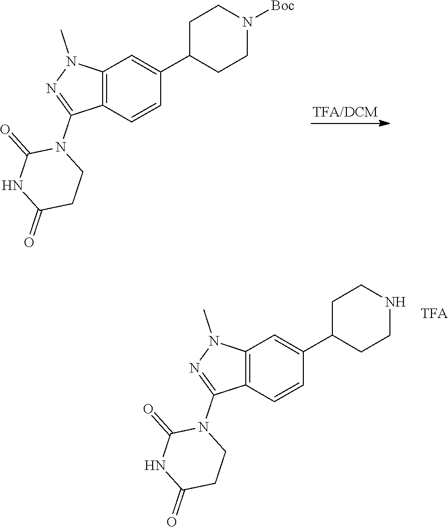 [SMILES: Cn1nc(N2CCC(=O)NC2=O)c2ccc(C3CCN(C(=O)OC(C)(C)C)CC3)cc21 ; Cn1nc(N2CCC(=O)NC2=O)c2ccc(C3CCNCC3)cc21 ; "TFA"]

A mixture of tert-butyl 4-(3-(2,4-dioxotetrahydropyrimidin-1(2H)-yl)-1-methyl-1H-indazol-6-yl)piperidine-1-carboxylate (100 mg, 0.25 mmol, 1.00 eq.) in TFA/DCM (0.5 mL/2.0 mL) was stirred at rt for 2 h. The mixture was concentrated to give the title compound as brown oil.

### Step 8: rac-tert-Butyl ((3R,4S)-1-((3-((4-(3-(2,4-dioxotetrahydropyrimidin-1(2H)-yl)-1-methyl-1H-indazol-6-yl)piperidin-1-yl)methyl)phenyl)sulfonyl)-3-fluoropiperidin-4-yl)carbamate

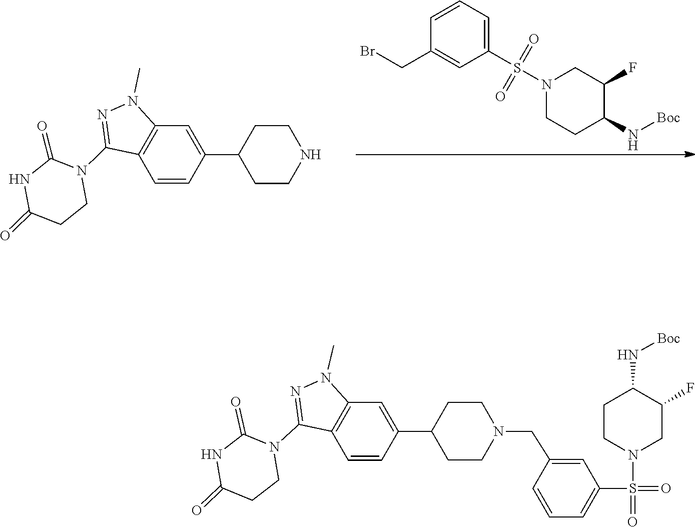 [SMILES: Cn1nc(N2CCC(=O)NC2=O)c2ccc(C3CCNCC3)cc21 ; CC(C)(C)OC(=O)N[C@H]1CCN(S(=O)(=O)c2cccc(CBr)c2)C[C@H]1F ; Cn1nc(N2CCC(=O)NC2=O)c2ccc(C3CCN(Cc4cccc(S(=O)(=O)N5CC[C@H](NC(=O)OC(C)(C)C)[C@H](F)C5)c4)CC3)cc21]

A mixture of 1-(1-methyl-6-(piperidin-4-yl)-1H-indazol-3-yl)dihydropyrimidine-2,4(1H,3H)-dione (82 mg, 0.25 mmol, 1.00 eq.), rac-tert-butyl ((3R,4S)-1-((3-(bromomethyl)phenyl)sulfonyl)-3-fluoropiperidin-4-yl)carbamate (115 mg, 0.25 mmol, 1.00 eq.) in DCM (4.0 mL), and TEA (76 mg, 0.75 mmol, 3.00 eq.) was stirred at 50° C. for 16 h. The mixture was filtered and the filtrate was concentrated. The residue was purified by column chromatography on silica gel (DCM:MeOH=15:1) to give the title compound as white solid.

### Step 9: rac-1-(6-(1-(3-(((3R,4S)-4-amino-3-fluoropiperidin-1-yl)sulfonyl)benzyl)piperidin-4-yl)-1-methyl-1H-indazol-3-yl)dihydropyrimidine-2,4(1H,3H)-dione

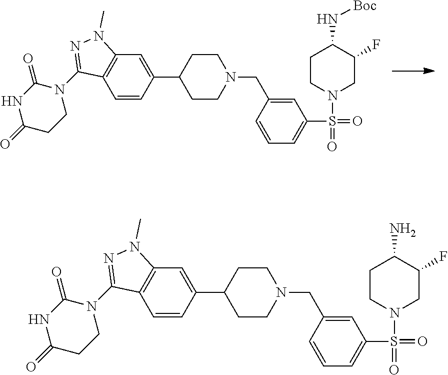 [SMILES: Cn1nc(N2CCC(=O)NC2=O)c2ccc(C3CCN(Cc4cccc(S(=O)(=O)N5CC[C@H](NC(=O)OC(C)(C)C)[C@H](F)C5)c4)CC3)cc21 ; Cn1nc(N2CCC(=O)NC2=O)c2ccc(C3CCN(Cc4cccc(S(=O)(=O)N5CC[C@H](N)[C@H](F)C5)c4)CC3)cc21]

A mixture of tert-butyl ((3R,4S)-1-((3-((4-(3-(2,4-dioxotetrahydropyrimidin-1(2H)-yl)-1-methyl-1H-indazol-6-yl)piperidin-1-yl)methyl)phenyl)sulfonyl)-3-fluoropiperidin-4-yl)carbamate (45 mg, 0.07 mmol, 1.00 eq.) in TFA/DCM (0.5 mL/2.0 mL) was stirred at rt for 2 h. The mixture was concentrated to give the title compound as its TFA salt as brown oil.

The following intermediates were synthesized by proceeding analogously as described in Reference 45.

| | | |
|---|---|---|
| Ref 45a | 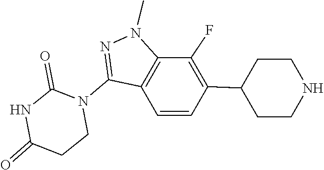 [SMILES: Cn1nc(N2CCC(=O)NC2=O)c2ccc(C3CCNCC3)c(F)c21] | 4-bromo-2,3-difluorobenzo- nitrile replaced 4-bromo-2- fluorobenzonitrile in Step 1 |
| Ref 45b | 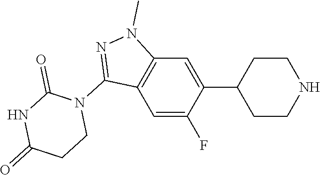 [SMILES: Cn1nc(N2CCC(=O)NC2=O)c2cc(F)c(C3CCNCC3)cc21] | 4-bromo-2,3-difluorobenzo- nitrile replaced 4-bromo-2- fluorobenzonitrile in Step 1 |

### Reference 46

### Synthesis of 3-((4-(Piperidin-4-yl)phenyl)amino)piperidine-2,6-dione

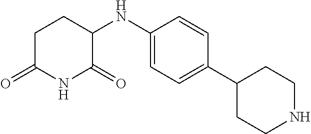 [SMILES: O=C1CCC(Nc2ccc(C3CCNCC3)cc2)C(=O)N1]

### Step 1: tert-Butyl 4-(4-nitrophenyl)-5,6-dihydropyridine-1(2H)-carboxylate

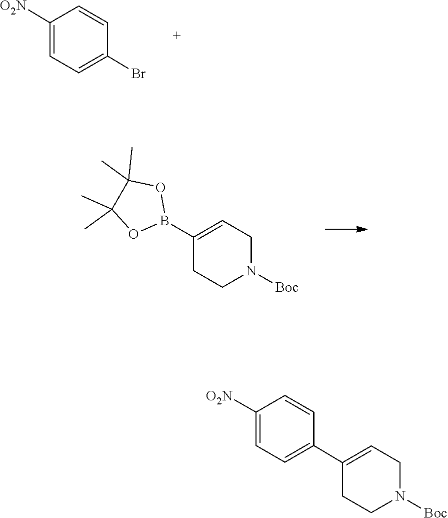 [SMILES: O=[N+]([O-])c1ccc(Br)cc1 ; CC(C)(C)OC(=O)N1CC=C(B2OC(C)(C)C(C)(C)O2)CC1 ; CC(C)(C)OC(=O)N1CC=C(c2ccc([N+](=O)[O-])cc2)CC1]

A mixture of 1-bromo-4-nitrobenzene (1.0 g, 4.95 mmol, 1.00 eq), tert-butyl 4-(4,4,5,5-tetramethyl-1,3,2-dioxaborolan-2-yl)-5,6-dihydropyridine-1(2H)-carboxylate (2.30 g, 7.43 mmol, 1.50 eq), K₂CO₃ (1.37 g, 9.90 mmol, 2.00 eq), and Pd(dppf)Cl₂ (724 mg, 0.99 mmol, 0.20 eq) in dioxane/H₂O (15 mL, 5/1) was stirred at 100° C. for 4 h. The mixture was filtered and extracted with EA. The combined organic layers were dried over anhydrous Na₂SO₄, filtered, and concentrated. The residue was purified by silica flash column PE/EtOAc (10:1) to give product as yellow solid.

### Step 2: tert-Butyl 4-(4-aminophenyl)piperidine-1-carboxylate

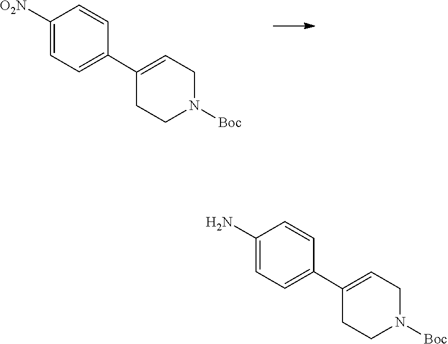 [SMILES: CC(C)(C)OC(=O)N1CC=C(c2ccc([N+](=O)[O-])cc2)CC1 ; CC(C)(C)OC(=O)N1CC=C(c2ccc(N)cc2)CC1]

A mixture of tert-butyl 4-(4-nitrophenyl)-5,6-dihydropyridine-1(2H)-carboxylate (1.20 g, 3.95 mmol, 1.00 eq), Pd/C (360 mg) in MeOH/THF (30 mL, 1:1) was stirred at 45° C. under H₂ overnight. The mixture was filtered and concentrated, and the residue was purified by silica flash column PE/EtOAc (3:1) to give product as yellow solid.

### Step 3: tert-Butyl 4-(4-((2,6-dioxopiperidin-3-yl)amino)phenyl)piperidine-1-carboxylate

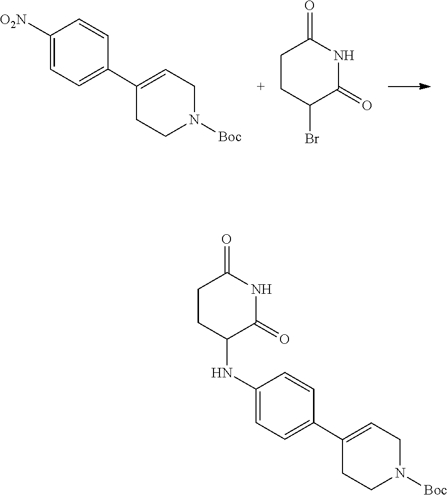 [SMILES: CC(C)(C)OC(=O)N1CC=C(c2ccc([N+](=O)[O-])cc2)CC1 ; O=C1CCC(Br)C(=O)N1 ; CC(C)(C)OC(=O)N1CC=C(c2ccc(NC3CCC(=O)NC3=O)cc2)CC1]

A mixture of tert-butyl 4-(4-aminophenyl)piperidine-1-carboxylate (332 mg, 1.20 mmol, 1.00 eq.), 3-bromopiperidine-2,6-dione (242 mg, 1.26 mmol, 1.05 eq.) and NaHCO₃ (302 mg, 3.60 mmol, 3.00 eq.) in DMF (4.0 mL) was stirred at 70° C. for overnight. The mixture was diluted with water and extracted with EA. The combined organic layers were dried over anhydrous Na₂SO₄, filtered, and then concentrated. The residue was purified by silica flash column PE/EtOAc (1:1) to give the title compound as yellow solid.

### Step 4: 3-((4-(Piperidin-4-yl)phenyl)amino)piperidine-2,6-dione

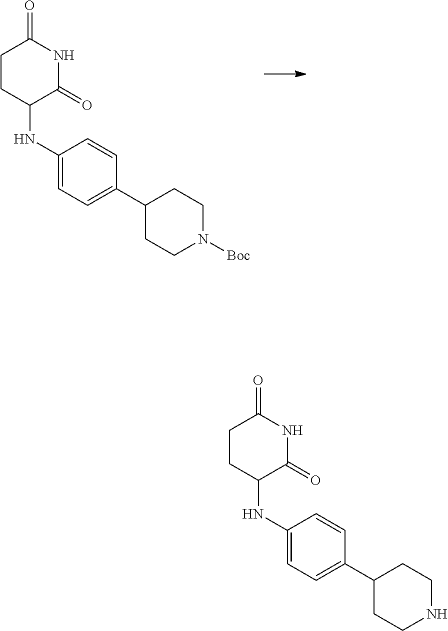 [SMILES: CC(C)(C)OC(=O)N1CCC(c2ccc(NC3CCC(=O)NC3=O)cc2)CC1 ; O=C1CCC(Nc2ccc(C3CCNCC3)cc2)C(=O)N1]

TFA (0.5 mL) was added to a mixture of tert-butyl 4-(4-((2,6-dioxopiperidin-3-yl)amino)phenyl)piperidine-1-carboxylate (100 mg, 0.26 mmol, 1.00 eq.) in DCM (2.0 mL) and the mixture was stirred at rt for 2 h. The solution was concentrated to give the title compound as a yellow solid.

### Reference 47

### Synthesis of 3-(4-(piperazin-1-yl)phenyl)piperidine-2,6-dione

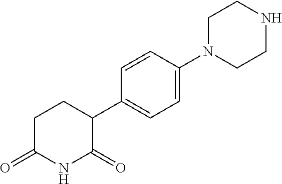 [SMILES: O=C1CCC(c2ccc(N3CCNCC3)cc2)C(=O)N1]

### Step 1: 2,6-Bis(benzyloxy)pyridine

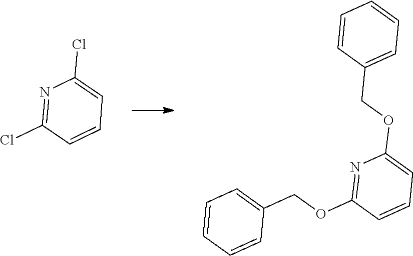 [SMILES: Clc1cccc(Cl)n1 ; c1ccc(COc2cccc(OCc3ccccc3)n2)cc1]

To a stirred solution of phenylmethanol (14.60 g, 135.14 mmol, 2.00 eq.) in THF (250.0 mL) were added t-BuOK (38.00 g, 337.84 mmol, 5.00 eq.) and 2,6-dichloropyridine (10.00 g, 67.57 mmol, 1.00 eq.). The mixture was stirred at 75° C. for 20 h under N₂. The mixture was diluted with water and extracted with EA, and the combined organic layers was washed with brine, dried over Na₂SO₄, concentrated to give the title compound as pale yellow solid.

### Step 2: 2,6-Bis(benzyloxy)-3-bromopyridine

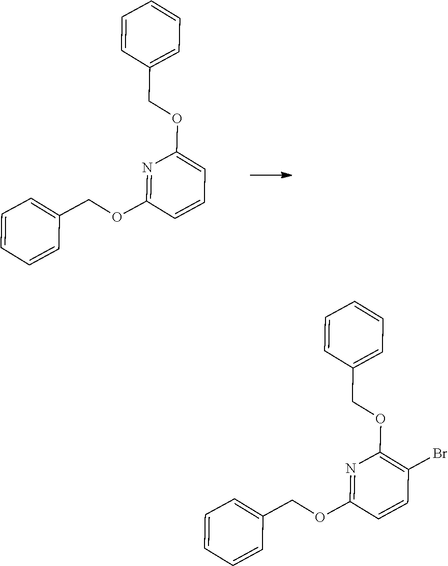 [SMILES: c1ccc(COc2cccc(OCc3ccccc3)n2)cc1 ; Brc1ccc(OCc2ccccc2)nc1OCc1ccccc1]

NBS (8.70 g, 0.05 mol, 0.95 eq.) was added to a stirred solution of 2,6-bis(benzyloxy)-pyridine (15.00 g, 0.05 mol, 1.00 eq.) in MeCN (100.0 mL) and the mixture was stirred at 80° C. for 4 h under N₂. The mixture was diluted with water and extracted with EA. The combined organic layers was washed with brine, dried over Na₂SO₄, concentrated to give the title compound as yellow solid.

### Step 3: 2,6-Bis(benzyloxy)-3-(4,4,5,5-tetramethyl-1,3,2-dioxaborolan-2-yl)pyridine

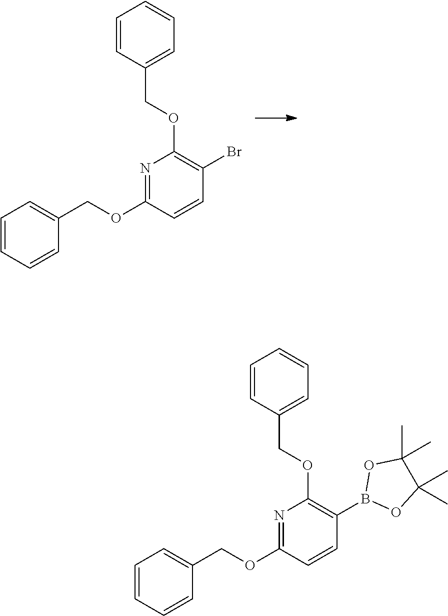 [SMILES: Brc1ccc(OCc2ccccc2)nc1OCc1ccccc1 ; CC1(C)OB(c2ccc(OCc3ccccc3)nc2OCc2ccccc2)OC1(C)C]

A mixture of 2,6-bis(benzyloxy)-3-bromopyridine (19.00 g, 0.05 mol, 1.00 eq.), 4,4,4′,4′,5,5,5′,5′-octamethyl-2,2′-bi (1,3,2-dioxaborolane) (19.60 g, 0.08 mol, 1.50 eq.), KOAc (10.00 g, 0.10 mol, 2.00 eq.), and Pd(dppf)Cl₂ (3.7 g, 5.00 mmol, 0.10 eq.) in 1,4-dioxane (200.0 mL) was stirred at 100° C. for 25 h under N₂. The mixture was diluted with water and extracted with EA, and the combined organic layer was washed with brine, dried over Na₂SO₄, and concentrated. The residue was purified by silica gel column chromatography eluting with EA:PE=0 to 100% to give title compound as yellow solid.

### Step 4: 2,6-Bis(benzyloxy)-3-(4-bromophenyl)pyridine

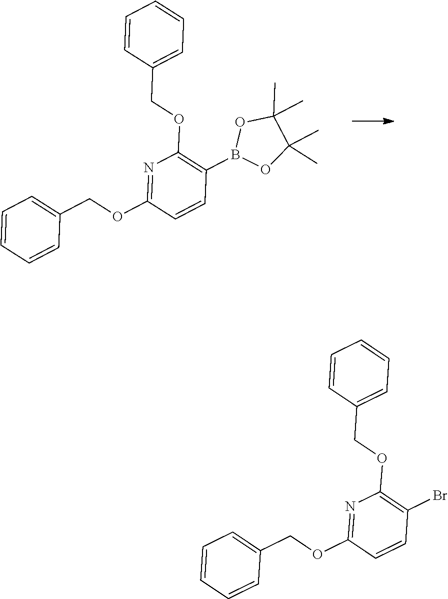 [SMILES: CC1(C)OB(c2ccc(OCc3ccccc3)nc2OCc2ccccc2)OC1(C)C ; Brc1ccc(OCc2ccccc2)nc1OCc1ccccc1]

A mixture of 2,6-bis(benzyloxy)-3-(4,4,5,5-tetramethyl-1,3,2-dioxaborolan-2-yl)pyridine (4.42 g, 10.60 mmol, 1.20 eq.), 1-bromo-4-iodobenzene (2.50 g, 8.83 mol, 1.00 eq.), K₃PO₄ (5.63 g, 26.50 mmol, 3.00 eq.), and Pd(PPh₃)₄ (510 mg, 0.44 mmol, 0.05 eq.) in 1,4-dioxane/H₂O=10:1 (40.0 mL) was stirred at 100° C. for 16 h under N₂. The mixture was diluted with water and extracted with EA, and the combined organic layer was washed with brine, dried over Na₂SO₄, and concentrated. The residue was purified by silica gel column chromatography eluting with EA:PE=0 to 100% to give the title compound as yellow solid.

### Step 5: tert-Butyl 4-(4-(2,6-bis(benzyloxy)pyridin-3-yl)phenyl)piperazine-1-carboxylate

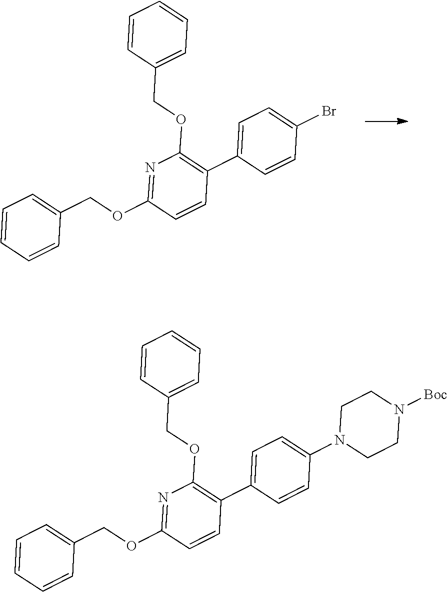 [SMILES: Brc1ccc(-c2ccc(OCc3ccccc3)nc2OCc2ccccc2)cc1 ; CC(C)(C)OC(=O)N1CCN(c2ccc(-c3ccc(OCc4ccccc4)nc3OCc3ccccc3)cc2)CC1]

A mixture of 2,6-bis(benzyloxy)-3-(4-bromophenyl)pyridine (500 mg, 1.12 mmol, 1.00 eq.), tert-butyl piperazine-1-carboxylate (417 mg, 2.24 mmol, 2.00 eq.), Cs₂CO₃ (730 mg, 2.24 mmol, 2.00 eq.), Pd₂(dba)₃ (51 mg, 0.06 mmol, 0.05 eq.), and Ruphos (52 mg, 0.11 mmol, 0.10 eq.) in toluene (15.0 mL) was stirred at 110° C. for 20 h under N₂. The mixture was diluted with water and extracted with EA, and the combined organic layer was washed with brine, dried over Na₂SO₄, concentrated. The residue was purified by silica gel column chromatography eluting with EA:PE=0 to 100% to give the title compound as yellow solid.

### Step 6: tert-Butyl 4-(4-(2,6-dioxopiperidin-3-yl)phenyl)piperazine-1-carboxylate

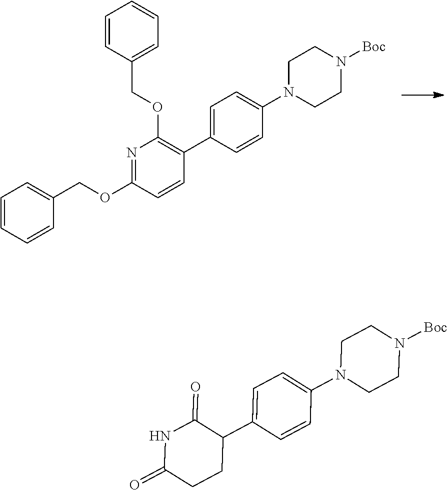 [SMILES: CC(C)(C)OC(=O)N1CCN(c2ccc(-c3ccc(OCc4ccccc4)nc3OCc3ccccc3)cc2)CC1 ; CC(C)(C)OC(=O)N1CCN(c2ccc(C3CCC(=O)NC3=O)cc2)CC1]

A mixture of tert-butyl 4-(4-(2,6-bis(benzyloxy)pyridin-3-yl)phenyl)piperazine-1-carboxylate (260 mg, 0.47 mmol, 1.00 eq.), 10% Pd/C (260 mg) in EA (5.0 mL) and 1,4-dioxane (5.0 mL) was stirred at r.t for 20 h under H₂. The mixture was filtered and the filtrate was concentrated to give the title compound as yellow oil.

### Step 7: 3-(4-(Piperazin-1-yl)phenyl)piperidine-2,6-dione

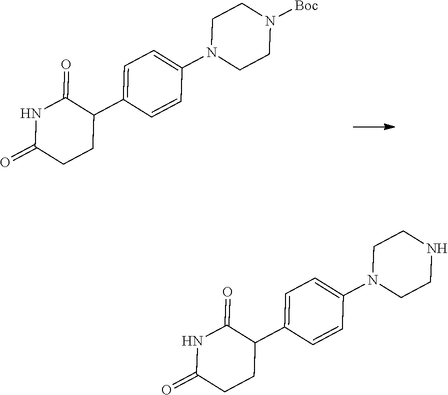 [SMILES: CC(C)(C)OC(=O)N1CCN(c2ccc(C3CCC(=O)NC3=O)cc2)CC1 ; O=C1CCC(c2ccc(N3CCNCC3)cc2)C(=O)N1]

TFA (0.5 mL) was added to a stirred solution of tert-butyl 4-(4-(2,6-dioxopiperidin-3-yl)phenyl)piperazine-1-carboxylate (160 mg, 0.43 mmol, 1.00 eq.) in DCM (2.0 mL) and the mixture was stirred at r.t for 2 h under N₂. The mixture was concentrated to give the title compound as its TFA salt as yellow oil.

### Reference 48

### Synthesis of 2-chloro-5-(difluoromethyl)pyrimidine

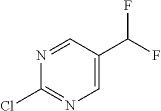

To a solution of 2-chloropyrimidine-5-carbaldehyde (250 mg, 1.60 mmol, 1.00 eq.) in DCM (3.0 mL) was added DAST (45 mg, 31.93 mmol, 20.00 eq.) at 0° C. and the mixture was stirred at r.t overnight. The mixture was diluted with water and extracted DCM. The organic layer was washed with brine, dried over Na₂SO₄, concentrated and purified by flash chromatography (PE:EA=10:1) to give the title compound as a white solid.

The following intermediates were prepared by proceeding analogously to Reference 48

| Ref# | Structure | Synthesis |
|---|---|---|
| Ref. 48a | 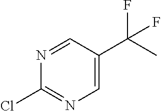 | 1-(2-chloropyrimidin-5-yl)ethan-1-one replaced 2-chloro-pyrimidine-5-carbalde- hyde |

### Reference 49

### Synthesis of 2-chloro-5-(difluoromethoxy)pyrimidine

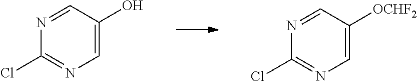 [SMILES: Oc1cnc(Cl)nc1 ; FC(F)Oc1cnc(Cl)nc1]

A mixture of 2-chloropyrimidin-5-ol (1.00 g, 7.69 mmol, 1.00 eq.), methyl 2-chloro-2,2-difluoroacetate (3.32 g, 23.08 mmol, 3.00 eq.) and Cs₂CO₃ (3.01 g, 9.23 mmol, 1.20 eq.) in DMF (10.0 mL) was stirred at 100° C. under N₂ 1 h. The mixture was poured into water, extracted with DCM, and the combined organic layers was dried over Na₂SO₄, and concentrated. Purification of the crude product with column chromatography on silica gel (PE:EA=20:1) gave the title compound as yellow oil.

### Reference 50

### Synthesis of 1-(6-(piperidin-4-yl)-1-(2,2,2-trifluoroethyl)-1H-indazol-3-yl)dihydropyrimidine-2,4(1H,3H)-dione

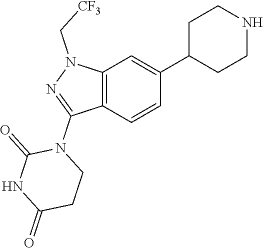 [SMILES: O=C1CCN(c2nn(CC(F)(F)F)c3cc(C4CCNCC4)ccc23)C(=O)N1]

### Step 1: 6-Bromo-1-(2,2,2-trifluoroethyl)-1H-indazol-3-amine

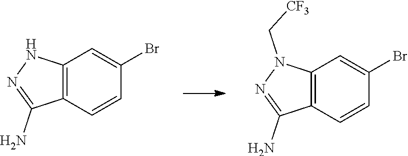 [SMILES: Nc1n[nH]c2cc(Br)ccc12 ; Nc1nn(CC(F)(F)F)c2cc(Br)ccc12]

NaH (2.10 g, 52.83 mmol, 2.00 eq.) was added to a stirred solution of 6-bromo-1H-indazol-3-amine (5.60 g, 26.42 mmol, 1.00 eq.) in DMF (20.0 mL) at 0° C. and the mixture was stirred at 0° C. for 1 h. 2,2,2-Trifluoroethyl trifluoromethanesulfonate (6.7 g, 29.06 mmol, 1.10 eq.) was added and the mixture was stirred at r.t. for 3 h under N₂. The mixture was poured into cold water and filtered. The solid was washed with water and dried to give the title compound as yellow solid.

### Step 2: 1-(6-(Piperidin-4-yl)-1-(2,2,2-trifluoroethyl)-1H-indazol-3-yl)dihydropyrimidine-2,4(1H,3H)-dione

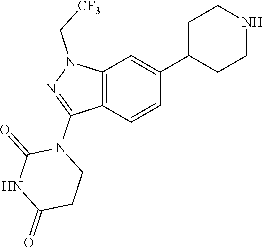 [SMILES: O=C1CCN(c2nn(CC(F)(F)F)c3cc(C4CCNCC4)ccc23)C(=O)N1]

The title compound was synthesized by proceeding analogously as described in Reference 45, Steps 2-7 with 6-bromo-1-(2,2,2-trifluoroethyl)-1H-indazol-3-amine replacing 6-bromo-1-methyl-1H-indazol-3-amine.

### Reference 51

### Synthesis of 1-(1-methyl-6-(piperazin-1-yl)-1H-indazol-3-yl)dihydropyrimidine-2,4(1H,3H)-dione

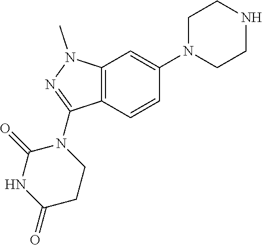 [SMILES: Cn1nc(N2CCC(=O)NC2=O)c2ccc(N3CCNCC3)cc21]

### Step 1: Benzyl 4-(4-cyano-3-fluorophenyl)piperazine-1-carboxylate

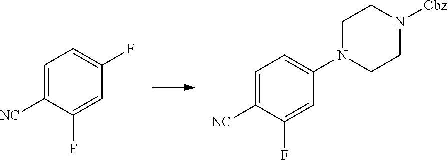 [SMILES: N#Cc1ccc(F)cc1F ; N#Cc1ccc(N2CCN(C(=O)OCc3ccccc3)CC2)cc1F]

A mixture of 2,4-difluorobenzonitrile (18.95 g, 136.20 mmol, 1.50 eq.), benzyl piperazine-1-carboxylate (20 g, 90.80 mmol, 1.00 eq.) and potassium carbonate (25.10 g, 181.6 mmol, 2.00 eq.) in ACN (200.0 mL) was stirred at 80° C. under N₂ for 16 h. The mixture was filtered and the filtrate was concentrated. The residue was purified by silica gel column chromatography eluting with PE/EtOAc (3:1) to give the title compound as white solid.

### Step 2: Benzyl 4-(3-amino-1H-indazol-6-yl)piperazine-1-carboxylate

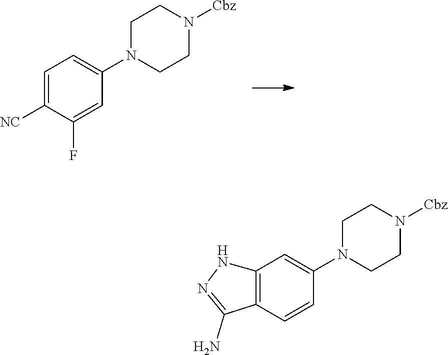 [SMILES: N#Cc1ccc(N2CCN(C(=O)OCc3ccccc3)CC2)cc1F ; Nc1n[nH]c2cc(N3CCN(C(=O)OCc4ccccc4)CC3)ccc12]

A mixture of benzyl 4-(4-cyano-3-fluorophenyl)piperazine-1-carboxylate (11.00 g, 32.40 mmol, 1.00 eq.) and N₂H₄/H₂O (10.14 g, 161.99 mmol, 5.00 eq) in BuOH (100.0 mL) was stirred at 100° C. under N₂ for 16 h. The mixture was concentrated and purified by flash chromatography to give the title compound as yellow solid.

### Step 3: Benzyl 4-(3-amino-1-methyl-1H-indazol-6-yl)piperazine-1-carboxylate

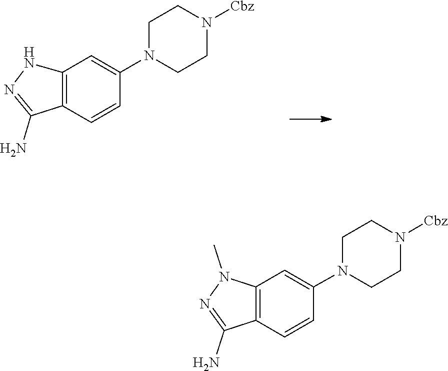 [SMILES: Nc1n[nH]c2cc(N3CCN(C(=O)OCc4ccccc4)CC3)ccc12 ; Cn1nc(N)c2ccc(N3CCN(C(=O)OCc4ccccc4)CC3)cc21]

To a solution of benzyl 4-(3-amino-1H-indazol-6-yl)piperazine-1-carboxylate (4.00 g, 11.40 mmol, 1.00 eq.) in dry DMF (50.0 mL) at 0° C. was added NaH (0.91 g, 22.80 mmol, 2.00 eq.) under N₂, and the mixture was stirred at rt for 30 min. The mixture was cooled to 0° C., CH₃I (1.78 g, 12.54 mmol, 1.10 eq.) in dry DMF (10.0 mL) was added dropwise, and the mixture was stirred for 3 h. The mixture was quenched with water, extracted with EA. The combined organic layers were washed with brine, dried with Na₂SO₄, and concentrated. The residue was purified by silica gel column chromatography eluting with DCM/MeOH=(50:1) to give the title compound as yellow solid.

### Step 4: Benzyl 4-(3-(2,4-dioxotetrahydropyrimidin-1(2H)-yl)-1-methyl-1H-indazol-6-yl)-piperazine-1-carboxylate

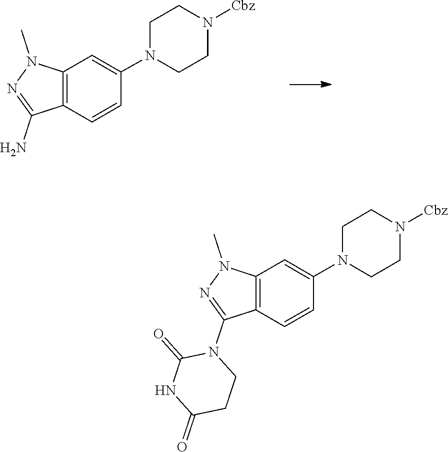 [SMILES: Cn1nc(N)c2ccc(N3CCN(C(=O)OCc4ccccc4)CC3)cc21 ; Cn1nc(N2CCC(=O)NC2=O)c2ccc(N3CCN(C(=O)OCc4ccccc4)CC3)cc21]

The title compound was synthesized by proceeding analogously as described in Reference 45, Steps 2-4 with benzyl 4-(3-amino-1-methyl-1H-indazol-6-yl)piperazine-1-carboxylate replacing 6-bromo-1-methyl-1H-indazol-3-amine.

### Step 5: 1-(1-Methyl-6-(piperazin-1-yl)-1H-indazol-3-yl)dihydropyrimidine-2,4(1H,3H)-dione

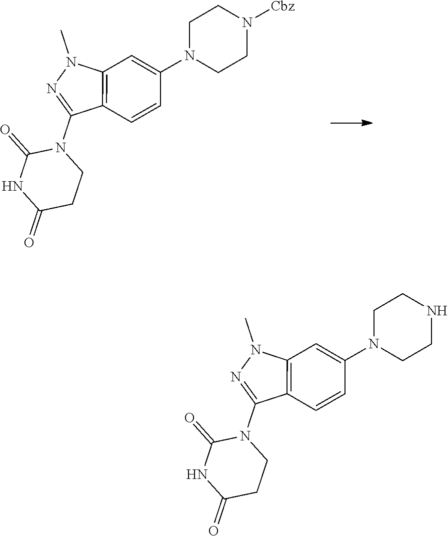 [SMILES: Cn1nc(N2CCC(=O)NC2=O)c2ccc(N3CCN(C(=O)OCc4ccccc4)CC3)cc21 ; Cn1nc(N2CCC(=O)NC2=O)c2ccc(N3CCNCC3)cc21]

A mixture of benzyl 4-[3-(2,4-dioxo-1,3-diazinan-1-yl)-1-methylindazol-6-yl]piperazine-1-carboxylate (500 mg, 1.08 mmol, 1.00 eq.), 10% Pd/C (400 mg) and ammonium formate (682 mg, 10.81 mmol, 10.00 eq.) in MeOH (20.0 mL) was stirred at 60° C. under N₂ for 16 h. The mixture was filtered and the filtrate was concentrated to give the title compound as white solid.

### Reference 52

### Synthesis of 1-(6-(piperazin-1-yl)-1-(2,2,2-trifluoroethyl)-1H-indazol-3-yl)-dihydropyrimidine-2,4(1H,3H)-dione

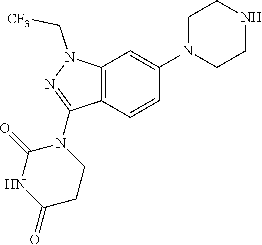 [SMILES: O=C1CCN(c2nn(CC(F)(F)F)c3cc(N4CCNCC4)ccc23)C(=O)N1]

### Step 1: Benzyl 4-(3-amino-1-(2,2,2-trifluoroethyl)-1H-indazol-6-yl)piperazine-1-carboxylate

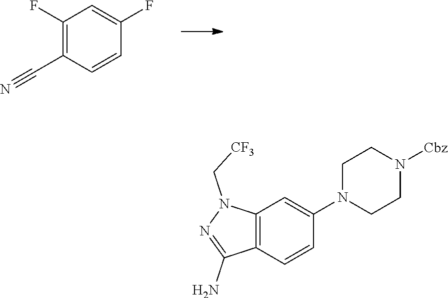 [SMILES: N#Cc1ccc(F)cc1F ; Nc1nn(CC(F)(F)F)c2cc(N3CCN(C(=O)OCc4ccccc4)CC3)ccc12]

The title compound was prepared by proceeding analogously as described in 51, with 2,2,2-trifluoroethyl trifluoromethanesulfonate replacing Mel in Step 3.

### Step 2: 3-((6-(4-((Benzyloxy) carbonyl)piperazin-1-yl)-1-(2,2,2-trifluoroethyl)-1H-indazol-3-yl)-amino) propanoic acid

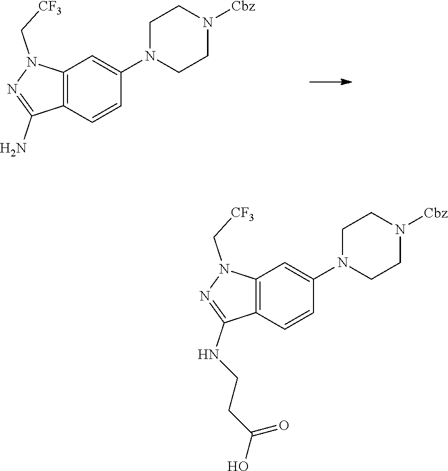 [SMILES: Nc1nn(CC(F)(F)F)c2cc(N3CCN(C(=O)OCc4ccccc4)CC3)ccc12 ; O=C(O)CCNc1nn(CC(F)(F)F)c2cc(N3CCN(C(=O)OCc4ccccc4)CC3)ccc12]

A mixture of benzyl 4-(3-amino-1-(2,2,2-trifluoroethyl)-1H-indazol-6-yl)piperazine-1-carboxylate (950 mg, 2.20 mmol, 1.00 eq.) and acrylic acid (237.6 mg, 3.30 mmol, 1.50 eq.) in toluene (10.0 mL) was stirred at 130° C. under N₂ for 12 h. The mixture was concentrated and the residue was purified by column chromatography (DCM:MeOH=80:1) to give the title compound as a yellow solid.

### Step 3: Benzyl 4-(3-(2,4-dioxotetrahydropyrimidin-1(2H)-yl)-1-(2,2,2-trifluoroethyl)-1H-indazol-6-yl)piperazine-1-carboxylate

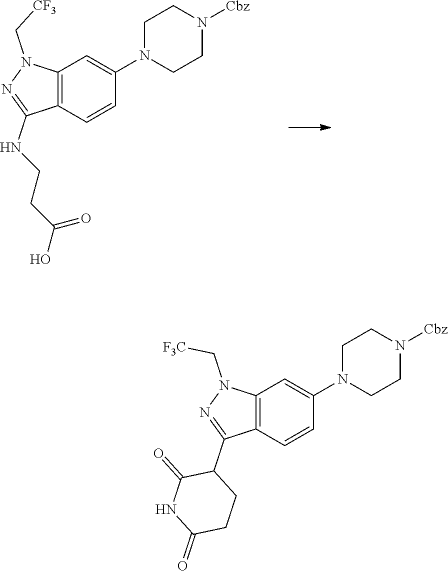 [SMILES: O=C(O)CCNc1nn(CC(F)(F)F)c2cc(N3CCN(C(=O)OCc4ccccc4)CC3)ccc12 ; O=C1CCC(c2nn(CC(F)(F)F)c3cc(N4CCN(C(=O)OCc5ccccc5)CC4)ccc23)C(=O)N1]

A mixture of 3-((6-(4-((benzyloxy) carbonyl)piperazin-1-yl)-1-(2,2,2-trifluoroethyl)-1H-indazol-3-yl)amino) propanoic acid (620 mg, 1.23 mmol, 1.00 eq.) and urea (369 mg, 6.15 mmol, 5.00 eq.) in AcOH (10.0 mL) was stirred at 120° C. under N₂ overnight. The mixture was diluted with water and extracted with EtOAc. The combined organic layers were washed with brine, dried over Na₂SO₄, filtered and concentrated. The residue was purified by column chromatography (DCM:MeOH=100:1) to give the title compound as a yellow solid.

### Step 4: 1-(6-(Piperazin-1-yl)-1-(2,2,2-trifluoroethyl)-1H-indazol-3-yl)dihydropyrimidine-2,4(1H,3H)-dione

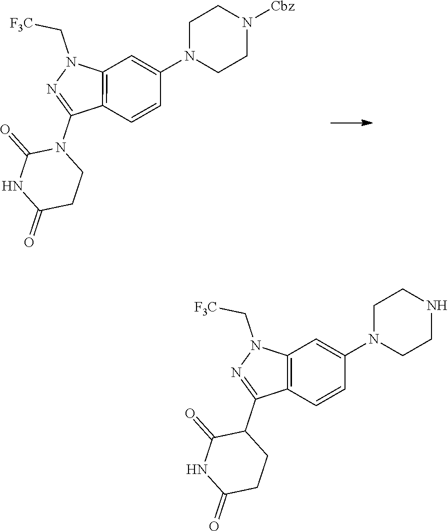 [SMILES: O=C1CCN(c2nn(CC(F)(F)F)c3cc(N4CCN(C(=O)OCc5ccccc5)CC4)ccc23)C(=O)N1 ; O=C1CCC(c2nn(CC(F)(F)F)c3cc(N4CCNCC4)ccc23)C(=O)N1]

A mixture of benzyl 4-(3-(2,4-dioxotetrahydropyrimidin-1(2H)-yl)-1-(2,2,2-trifluoroethyl)-1H-indazol-6-yl)piperazine-1-carboxylate (280 mg, 0.52 mmol, 1.00 eq.), HCOONH₄ (312 mg, 5.20 mmol, 10.0 eq.) and 10% Pd/C (100 mg) in THF/MeOH (5.0 mL/5.0 mL) was stirred at 60° C. under N₂ overnight. The mixture was concentrated and the residue was purified by column chromatography (DCM:MeOH=12:1) to give the title compound as a yellow solid.

### Reference 53

### Synthesis of 1-(6-(3,3-difluoropiperidin-4-yl)-1-methyl-1H-indazol-3-yl)dihydropyrimidine-2,4(1H,3H)-dione 2,2,2-trifluoroacetate

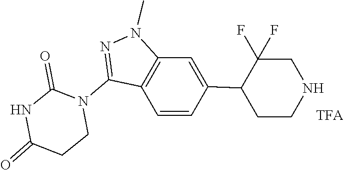 [SMILES: Cn1nc(N2CCC(=O)NC2=O)c2ccc(C3CCNCC3(F)F)cc21]

### Step 1: 1-(1-Methyl-6-(4,4,5,5-tetramethyl-1,3,2-dioxaborolan-2-yl)-1H-indazol-3-yl)dihydropyrimidine-2,4(1H,3H)-dione

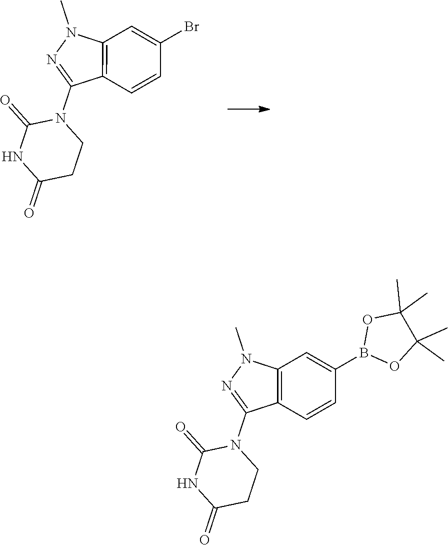 [SMILES: Cn1nc(N2CCC(=O)NC2=O)c2ccc(Br)cc21 ; Cn1nc(N2CCC(=O)NC2=O)c2ccc(B3OC(C)(C)C(C)(C)O3)cc21]

A mixture of 1-(6-bromo-1-methyl-1H-indazol-3-yl)dihydropyrimidine-2,4(1H,3H)-dione (626 mg, 2.00 mmol, 1.00 eq.), bis(pinacolato)diboron (762 mg, 3.00 mmol, 1.50 eq.), KOAc (589 mg, 6.00 mmol, 3.00 eq.) and Pd(dppf)Cl₂ (146 mg, 0.20 mmol, 0.10 eq.) in 1,4-dioxane (10 mL) was stirred at 85° C. under N₂ overnight. The mixture was concentrated and purified by column chromatography on silica gel (DCM:MeOH=100:1) to give the title compound as a yellow solid.

### Step 2: tert-Butyl 3,3-difluoro-4-(((trifluoromethyl)sulfonyl)oxy)-3,6-dihydropyridine-1(2H)-carboxylate

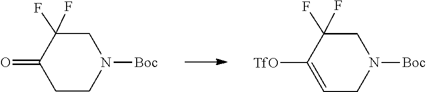 [SMILES: CC(C)(C)OC(=O)N1CCC(=O)C(F)(F)C1 ; CC(C)(C)OC(=O)N1CC=C(OS(=O)(=O)C(F)(F)F)C(F)(F)C1]

Tf₂O (3.03 g, 10.77 mmol, 1.50 eq.) was added to a mixture of tert-butyl 3,3-difluoro-4-oxopiperidine-1-carboxylate (1.68 g, 7.18 mmol, 1.00 eq.) and DIEA (5.56 g, 43.08 mmol, 6.00 eq.) in DCM (20.0 mL). The mixture was stirred at −10° C. under N₂ and then stirred at r.t. overnight. The mixture was concentrated in vacuo and purified by column chromatography on silica gel (PE:EA=10:1) to give the title compound as a brown oil.

### Step 3: tert-Butyl 4-(3-(2,4-dioxotetrahydropyrimidin-1(2H)-yl)-1-methyl-1H-indazol-6-yl)-3,3-difluoro-3,6-dihydropyridine-1(2H)-carboxylate

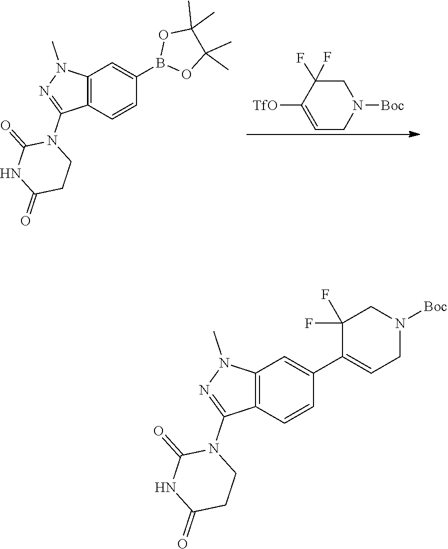 [SMILES: Cn1nc(N2CCC(=O)NC2=O)c2ccc(B3OC(C)(C)C(C)(C)O3)cc21 ; CC(C)(C)OC(=O)N1CC=C(OS(=O)(=O)C(F)(F)F)C(F)(F)C1 ; Cn1nc(N2CCC(=O)NC2=O)c2ccc(C3=CCN(C(=O)OC(C)(C)C)CC3(F)F)cc21]

A mixture of 1-(1-methyl-6-(4,4,5,5-tetramethyl-1,3,2-dioxaborolan-2-yl)-1H-indazol-3-yl)dihydropyrimidine-2,4(1H,3H)-dione (800 mg, 2.00 mmol, 1.00 eq.), tert-butyl 3,3-difluoro-4-(((trifluoromethyl)sulfonyl)oxy)-3,6-dihydropyridine-1(2H)-carboxylate (1.10 g, 3.00 mmol, 1.50 eq.), Na₂CO₃ (636 mg, 6.00 mmol, 3.00 eq.), Pd(dppf)Cl₂ (146 mg, 0.20 mmol, 0.1 eq.) and H₂O (2.5 mL) in 1,4-dioxane (10.0 mL) was stirred at 55° C. under N₂ overnight. The mixture was concentrated and the residue was purified by column chromatography on silica gel (DCM:MeOH=120:1) to give the title compound as a yellow solid.

### Step 4: tert-Butyl 4-(3-(2,4-dioxotetrahydropyrimidin-1(2H)-yl)-1-methyl-1H-indazol-6-yl)-3,3-difluoropiperidine-1-carboxylate

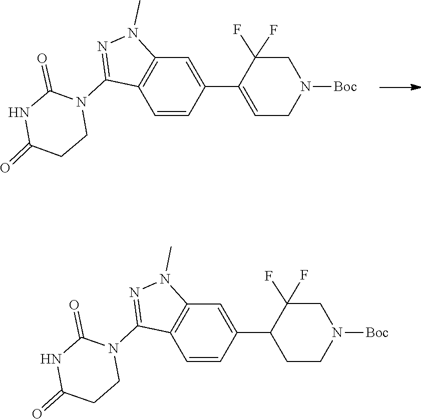 [SMILES: Cn1nc(N2CCC(=O)NC2=O)c2ccc(C3=CCN(C(=O)OC(C)(C)C)CC3(F)F)cc21 ; Cn1nc(N2CCC(=O)NC2=O)c2ccc(C3CCN(C(=O)OC(C)(C)C)CC3(F)F)cc21]

A mixture of tert-butyl 4-(3-(2,4-dioxotetrahydropyrimidin-1(2H)-yl)-1-methyl-1H-indazol-6-yl)-3,3-difluoro-3,6-dihydropyridine-1(2H)-carboxylate (940 mg, 2.00 mmol, 1.00 eq.), 10% Pd/C (900 mg) and Pd(OH)₂ (900 mg) in MeOH (10.0 mL) was stirred at 50° C. under H₂ (50 PSI) overnight. The mixture was filtered and the filtrate was concentrated. The residue was purified by column chromatography on silica gel (DCM:MeOH=1:1) to give the title as yellow solid.

### Step 5: 1-(6-(3,3-Difluoropiperidin-4-yl)-1-methyl-1H-indazol-3-yl)dihydropyrimidine-2,4(1H,3H)-dione 2,2,2-trifluoroacetate

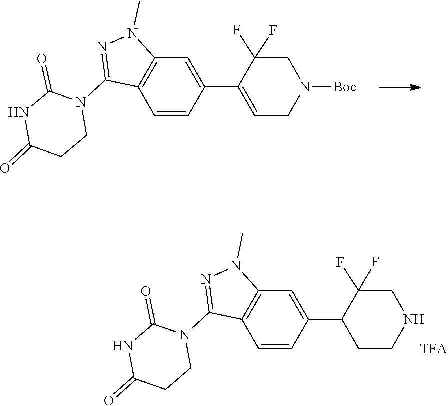 [SMILES: Cn1nc(N2CCC(=O)NC2=O)c2ccc(C3=CCN(C(=O)OC(C)(C)C)CC3(F)F)cc21 ; Cn1nc(N2CCC(=O)NC2=O)c2ccc(C3CCNCC3(F)F)cc21]

A mixture of tert-butyl 4-(3-(2,4-dioxotetrahydropyrimidin-1(2H)-yl)-1-methyl-1H-indazol-6-yl)-3,3-difluoropiperidine-1-carboxylate (102 mg, 0.22 mmol, 1.00 eq.) in TFA/DCM (0.5 mL/2.0 mL) was stirred at rt for 2 h. The mixture was concentrated to give the title compound as brown oil.

### Reference 54

### Synthesis of 1-(6-(1-(3-((4-aminopiperidin-1-yl)sulfonyl)benzyl)piperidin-4-yl)-1-methyl-1H-indazol-3-yl)dihydropyrimidine-2,4(1H,3H)-dione 2,2,2-trifluoroacetate

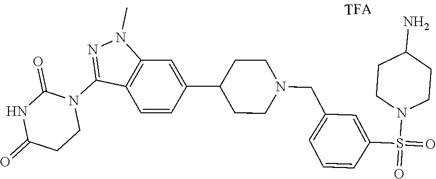 [SMILES: Cn1nc(N2CCC(=O)NC2=O)c2ccc(C3CCN(Cc4cccc(S(=O)(=O)N5CCC(N)CC5)c4)CC3)cc21]

### Step 1: 3-(Bromomethyl)benzenesulfonyl chloride

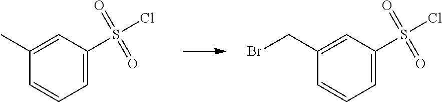

NBS (8.22 g, 46.16 mol, 1.10 eq.) and benzoyl peroxide (1.46 g, 4.20 mol, 0.01 eq.) were added to a stirred solution of 3-methylbenzenesulfonyl chloride (8.00 g, 41.96 mol, 1.00 eq.) in CC14 (80.0 mL) and the mixture was stirred at 80° C. for 12 h. The mixture was filtered, and the filtrate was concentrated to give title compound as white oil, which was used to next step without further purification.

### Step 2: tert-Butyl (1-((3-(bromomethyl)phenyl)sulfonyl)piperidin-4-yl)carbamate

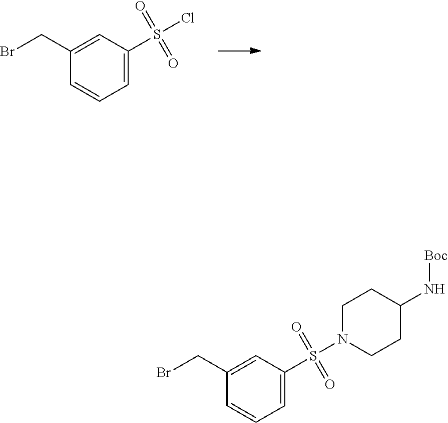 [SMILES: O=S(=O)(Cl)c1cccc(CBr)c1 ; CC(C)(C)OC(=O)NC1CCN(S(=O)(=O)c2cccc(CBr)c2)CC1]

3-(Bromomethyl)benzenesulfonyl chloride (5.64 g, 21.25 mol, 1.00 eq.) in THF (20.0 mL) was added to a stirred solution of tert-butyl piperidin-4-ylcarbamate (3.83 g, 19.13 mol, 0.90 eq.), TEA (4.30 g, 42.50 mmol, 2.00 eq.) in THF (40.0 mL) at 0° C. and the mixture was stirred at RT for 12 h. The mixture was quenched with H₂O and extracted with DCM. The combined organic layer was washed with water, dried over anhydrous Na₂SO₄, filtered, and then concentrated. The residue was purified by silica gel column chromatography, eluted with PE/EA (3:1), to afford the title compound as white solid.

### Step 3: tert-Butyl (1-((3-((4-(3-(2,4-dioxotetrahydropyrimidin-1(2H)-yl)-1-methyl-1H-indazol-6-yl)piperidin-1-yl)methyl)phenyl)sulfonyl)piperidin-4-yl)carbamate

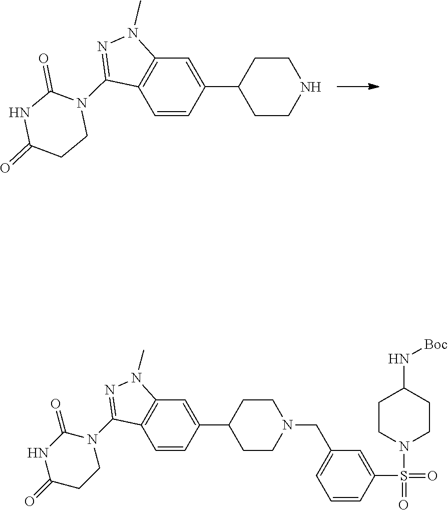 [SMILES: Cn1nc(N2CCC(=O)NC2=O)c2ccc(C3CCNCC3)cc21 ; Cn1nc(N2CCC(=O)NC2=O)c2ccc(C3CCN(Cc4cccc(S(=O)(=O)N5CCC(NC(=O)OC(C)(C)C)CC5)c4)CC3)cc21]

A mixture of 1-(1-methyl-6-(piperidin-4-yl)-1H-indazol-3-yl)dihydropyrimidine-2,4(1H,3H)-dione 2,2,2-trifluoroacetate (50 mg, 0.15 mmol, 1.00 eq.) and tert-butyl (1-((3-(bromomethyl)-phenyl)sulfonyl)piperidin-4-yl)carbamate (99 mg, 0.23 mmol, 1.50 eq.), TEA (45 mg, 0.45 mmol, 3.00 eq.) in THF (5.0 mL) was stirred at 55° C. overnight. The mixture was diluted with water and extracted with DCM, and the combined organic layer was washed with brine, dried over Na₂SO₄, concentrated, The residue was purified by flash chromatography (DCM:MeOH=20:1) to give title compound as a yellow solid.

### Step 4: 1-(6-(1-(3-((4-Aminopiperidin-1-yl)sulfonyl)benzyl)piperidin-4-yl)-1-methyl-1H-indazol-3-yl)dihydropyrimidine-2,4(1H,3H)-dione 2,2,2-trifluoroacetate

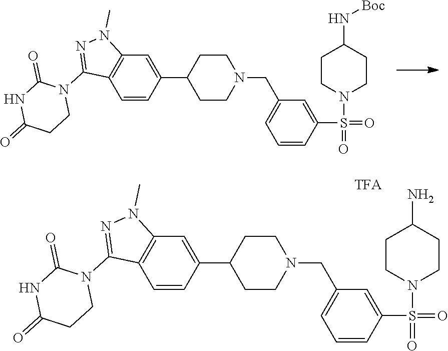 [SMILES: Cn1nc(N2CCC(=O)NC2=O)c2ccc(C3CCN(Cc4cccc(S(=O)(=O)N5CCC(NC(=O)OC(C)(C)C)CC5)c4)CC3)cc21 ; Cn1nc(N2CCC(=O)NC2=O)c2ccc(C3CCN(Cc4cccc(S(=O)(=O)N5CCC(N)CC5)c4)CC3)cc21]

A mixture of tert-butyl (1-((3-((4-(3-(2,4-dioxotetrahydropyrimidin-1(2H)-yl)-1-methyl-1H-indazol-6-yl)piperidin-1-yl)methyl)phenyl)sulfonyl)piperidin-4-yl)carbamate (50 mg, 0.07 mmol, 1.00 eq.) in TFA/DCM (0.5 mL/2.0 mL) was stirred at r.t. for 2 h. The mixture was concentrated to give the title compound as brown oil.

The following intermediates were prepared by proceeding analogously to Reference 54.

| Ref# | Structure | Synthesis |
|---|---|---|
| Ref. 54a | 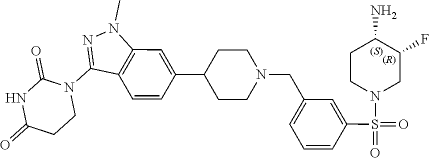 | tert-butyl ((3R,4S)-3-fluoropiper- idin-4-yl)carbamate replaced tert- butyl piperidin-4-ylcarbamate in Step 2 |
| Ref. 54b | 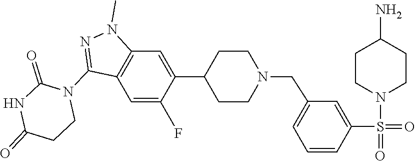 [SMILES: Cn1nc(N2CCC(=O)NC2=O)c2cc(F)c(C3CCN(Cc4cccc(S(=O)(=O)N5CCC(N)CC5)c4)CC3)cc21] | Ref 45b replaced 1-(1-methyl-6- (piperidin-4-yl)-1H-indazol-3- yl)dihydropyrimidine-2,4(1H, 3H)-dione 2,2,2-trifluoroacetate in Step 3 |
| Ref. 54c | 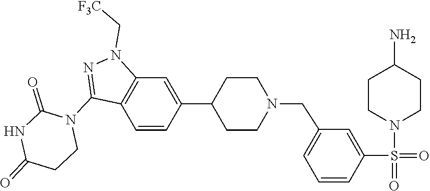 [SMILES: NC1CCN(S(=O)(=O)c2cccc(CN3CCC(c4ccc5c(N6CCC(=O)NC6=O)nn(CC(F)(F)F)c5c4)CC3)c2)CC1] | Ref 50 replaced 1-(1-methyl-6- (piperidin-4-yl)-1H-indazol-3- yl)dihydropyrimidine-2,4(1H, 3H)-dione 2,2,2-trifluoroacetate in Step 3 |
| Ref. 54d | 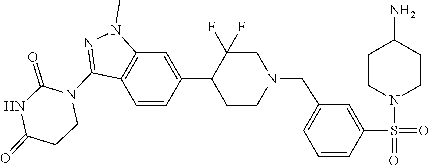 [SMILES: Cn1nc(N2CCC(=O)NC2=O)c2ccc(C3CCN(Cc4cccc(S(=O)(=O)N5CCC(N)CC5)c4)CC3(F)F)cc21] | Ref 53 replaced 1-(1-methyl-6- (piperidin-4-yl)-1H-indazol-3- yl)dihydropyrimidine-2,4(1H, 3H)-dione 2,2,2-trifluoroacetate in Step 3 |

### Reference 55

### Synthesis of 1-(6-(1-((1-((4-aminopiperidin-1-yl)sulfonyl)piperidin-4-yl)methyl)piperidin-4-yl)-1-methyl-1H-indazol-3-yl)dihydropyrimidine-2,4(1H,3H)-dione 2,2,2-trifluoroacetate

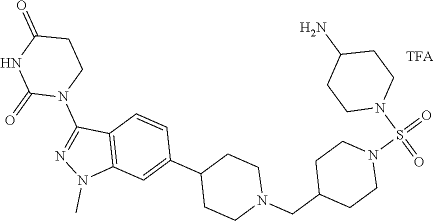 [SMILES: Cn1nc(N2CCC(=O)NC2=O)c2ccc(C3CCN(CC4CCN(S(=O)(=O)N5CCC(N)CC5)CC4)CC3)cc21]

### Step 1: tert-Butyl 4-((4-(3-(2,4-dioxotetrahydropyrimidin-1(2H)-yl)-1-methyl-1H-indazol-6-yl)piperidin-1-yl)methyl)piperidine-1-carboxylate

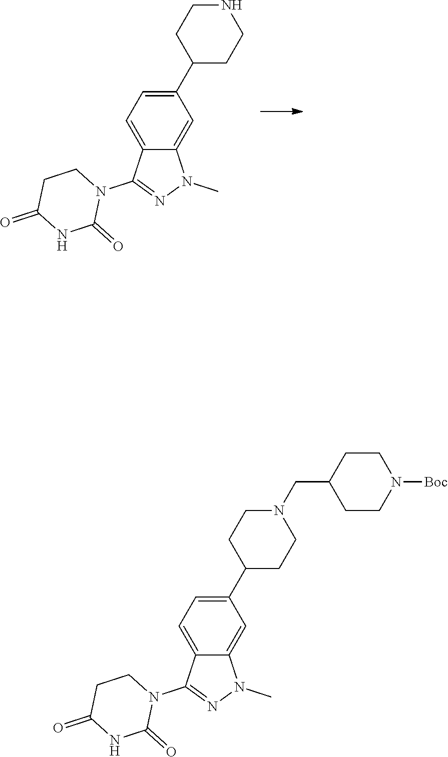 [SMILES: Cn1nc(N2CCC(=O)NC2=O)c2ccc(C3CCNCC3)cc21 ; Cn1nc(N2CCC(=O)NC2=O)c2ccc(C3CCN(CC4CCN(C(=O)OC(C)(C)C)CC4)CC3)cc21]

A mixture of 1-(1-methyl-6-(piperidin-4-yl)-1H-indazol-3-yl)dihydropyrimidine-2,4(1H,3H)-dione (500 mg, 1.40 mmol, 1.00 eq.), AcOH (3 drops), tert-butyl 4-formylpiperidine-1-carboxylate (300 mg, 1.40 mmol, 1.00 eq.) in THF (5.0 mL) and DMF (2.5 mL) was stirred at 45° C. for 45 min. The solution was cooled to r.t, NaBH₃CN (168 mg, 2.80 mmol, 2.00 eq.) was added and the mixture was stirred at r.t for 2 h. The resulting mixture was diluted with water and extracted with EtOAc. The combined organic layer was washed with brine, dried over anhydrous Na₂SO₄ and concentrated. The residue was purified by column chromatography, eluted with DCM/MeOH (20:1), to afford the title compound as a white solid.

### Step 2: 1-(1-Methyl-6-(1-(piperidin-4-ylmethyl)piperidin-4-yl)-1H-indazol-3-yl)dihydropyrimidine-2,4(1H,3H)-dione

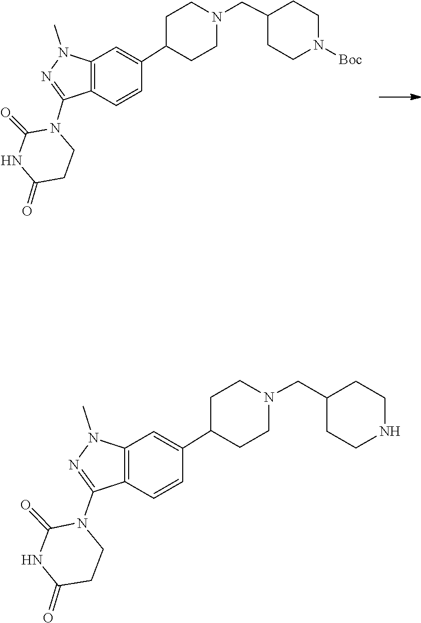 [SMILES: Cn1nc(N2CCC(=O)NC2=O)c2ccc(C3CCN(CC4CCN(C(=O)OC(C)(C)C)CC4)CC3)cc21 ; Cn1nc(N2CCC(=O)NC2=O)c2ccc(C3CCN(CC4CCNCC4)CC3)cc21]

A mixture of tert-butyl 4-((4-(3-(2,4-dioxotetrahydropyrimidin-1(2H)-yl)-1-methyl-1H-indazol-6-yl)piperidin-1-yl)methyl)piperidine-1-carboxylate (200 mg, 0.38 mmol, 1.00 eq.) in TFA/DCM (0.5 mL/2.0 mL) was stirred at rt for 2 h. The mixture was concentrated to give the title compound as brown oil.

### Step 3: tert-Butyl (1-((4-((4-(3-(2,4-dioxotetrahydropyrimidin-1(2H)-yl)-1-methyl-1H-indazol-6-yl)piperidin-1-yl)methyl)piperidin-1-yl)sulfonyl)piperidin-4-yl)carbamate

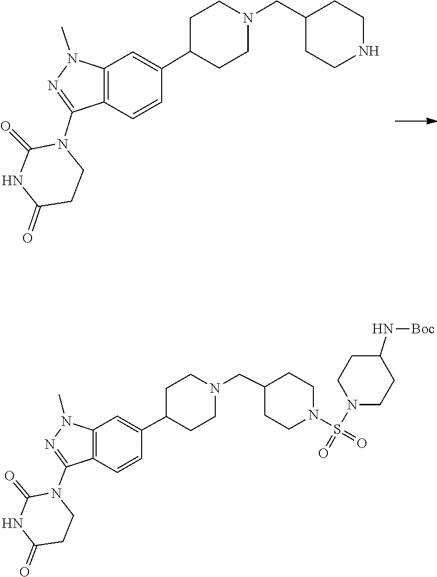 [SMILES: Cn1nc(N2CCC(=O)NC2=O)c2ccc(C3CCN(CC4CCNCC4)CC3)cc21 ; Cn1nc(N2CCC(=O)NC2=O)c2ccc(C3CCN(CC4CCN(S(=O)(=O)N5CCC(NC(=O)OC(C)(C)C)CC5)CC4)CC3)cc21]

tert-Butyl (1-(chlorosulfonyl)piperidin-4-yl)carbamate (59 mg, 0.20 mmol, 1.05 eq.) in DCM (2.0 mL) was added to a mixture of 1-(1-methyl-6-(1-(piperidin-4-ylmethyl)piperidin-4-yl)-1H-indazol-3-yl)dihydropyrimidine-2,4(1H,3H)-dione (80 mg, 0.19 mmol, 1.00 eq.), TEA (38 mg, 0.38 mmol, 2.00 eq.) in DCM (2.0 mL) at 0° C., and the mixture was stirred at RT for 12 h. The mixture was quenched with H₂O and then extracted with DCM. The combined organic layer was washed with water, dried over anhydrous Na₂SO₄, filtered, and then concentrated. The residue was purified by silica gel column chromatography, eluted with DCM/MeOH (20:1), to afford the title compound as white solid.

### Step 4: 1-(6-(1-((1-((4-Aminopiperidin-1-yl)sulfonyl)piperidin-4-yl)methyl)piperidin-4-yl)-1-methyl-1H-indazol-3-yl)dihydropyrimidine-2,4(1H,3H)-dione 2,2,2-trifluoroacetate

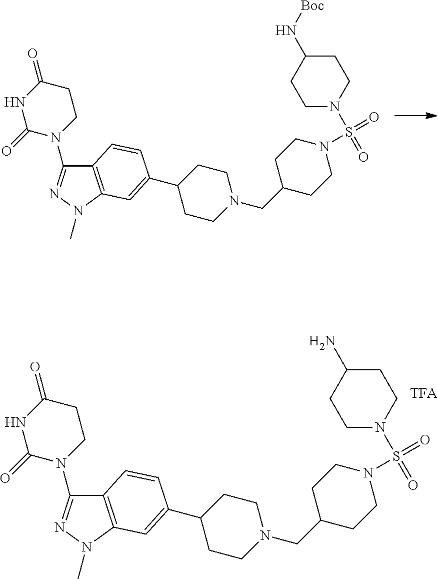 [SMILES: Cn1nc(N2CCC(=O)NC2=O)c2ccc(C3CCN(CC4CCN(S(=O)(=O)N5CCC(NC(=O)OC(C)(C)C)CC5)CC4)CC3)cc21 ; Cn1nc(N2CCC(=O)NC2=O)c2ccc(C3CCN(CC4CCN(S(=O)(=O)N5CCC(N)CC5)CC4)CC3)cc21]

A mixture of tert-butyl (1-((4-((4-(3-(2,4-dioxotetrahydropyrimidin-1(2H)-yl)-1-methyl-1H-indazol-6-yl)piperidin-1-yl)methyl)piperidin-1-yl)sulfonyl)piperidin-4-yl)carbamate (27 mg, 0.039 mmol, 1.00 eq.) in TFA/DCM (0.5 mL/2.0 mL) was stirred at rt for 2 h. The mixture was concentrated to give the title compound as yellow oil.

### Reference 56

### Synthesis of 1-(6-(1-((1-(3-((4-aminopiperidin-1-yl)sulfonyl)phenyl)piperidin-4-yl)methyl)piperidin-4-yl)-1-methyl-1H-indazol-3-yl)dihydropyrimidine-2,4(1H,3H)-dione 2,2,2-trifluoroacetate

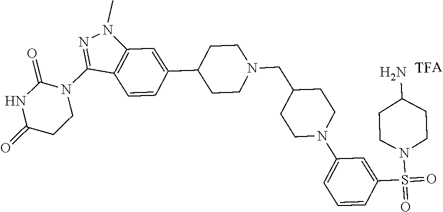 [SMILES: Cn1nc(N2CCC(=O)NC2=O)c2ccc(C3CCN(CC4CCN(c5cccc(S(=O)(=O)N6CCC(N)CC6)c5)CC4)CC3)cc21]

### Step 1: tert-Butyl (1-((3-bromophenyl)sulfonyl)piperidin-4-yl)carbamate

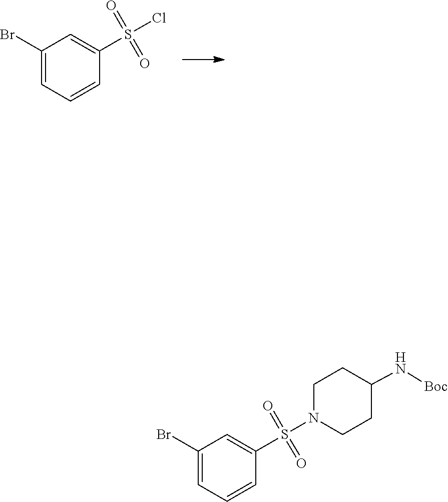 [SMILES: O=S(=O)(Cl)c1cccc(Br)c1 ; CC(C)(C)OC(=O)NC1CCN(S(=O)(=O)c2cccc(Br)c2)CC1]

3-Bromobenzene-1-sulfonyl chloride (5.0 g, 19.57 mmol, 1.00 eq.) in THF (50.0 mL) was added to a stirred solution of tert-butyl piperidin-4-ylcarbamate (4.12 g, 20.55 mmol, 1.05 eq.) in THF (50 mL) and TEA (2.18 g, 21.53 mmol, 1.10 eq.) dropwise at −10° C., and the mixture was stirred at rt for 2 h. The mixture was concentrated and the residue was taken into water/acetonitrile=1:1 and stirred for 1 h. The mixture was filtered, and the cake was washed with water and acetonitrile and dried to give the title compound a white solid.

### Step 2: tert-Butyl (1-((3-(4-(hydroxymethyl)piperidin-1-yl)phenyl)sulfonyl)piperidin-4-yl)carbamate

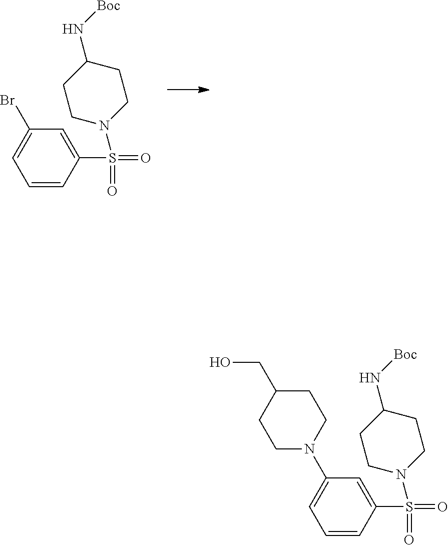 [SMILES: CC(C)(C)OC(=O)NC1CCN(S(=O)(=O)c2cccc(Br)c2)CC1 ; CC(C)(C)OC(=O)NC1CCN(S(=O)(=O)c2cccc(N3CCC(CO)CC3)c2)CC1]

A mixture of tert-butyl (1-((3-bromophenyl)sulfonyl)piperidin-4-yl)carbamate (3.00 g, 7.18 mmol, 1.00 eq.), K₂CO₃ (3.47 g, 25.1 mmol, 3.50 eq.), L-proline (248 mg, 2.15 mmol, 0.3 eq.), CuI (273 mg, 1.44 mmol, 0.2 eq.) and piperidin-4-ylmethanol (1.07 g, 9.33 mmol, 1.30 eq.) in DMSO (100.0 mL) was stirred at 100° C. for 12 h. The mixture was diluted with water and extracted with DCM. The combined organic layer was washed with brine, dried over anhydrous Na₂SO₄ and concentrated. The residue was purified by silica gel column chromatography, eluted with PE/EA=1:1, to afford the title compound as white solid.

### Step 3: tert-Butyl (1-((3-(4-formylpiperidin-1-yl)phenyl)sulfonyl)piperidin-4-yl)carbamate

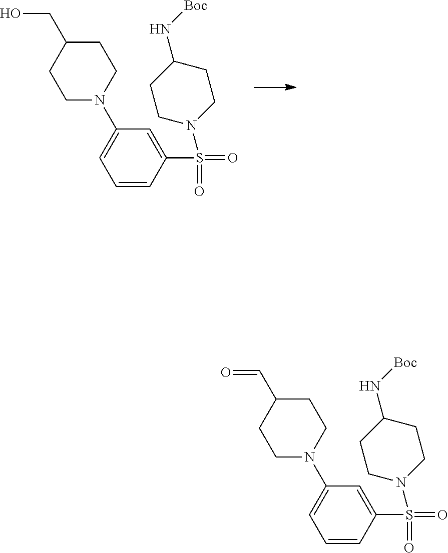 [SMILES: CC(C)(C)OC(=O)NC1CCN(S(=O)(=O)c2cccc(N3CCC(CO)CC3)c2)CC1 ; CC(C)(C)OC(=O)NC1CCN(S(=O)(=O)c2cccc(N3CCC(C=O)CC3)c2)CC1]

Dess-Martin (2.53 g, 5.96 mmol, 2.00 eq.) was added to a stirred solution of tert-butyl (1-((3-(4-(hydroxymethyl)piperidin-1-yl)phenyl)sulfonyl)piperidin-4-yl)carbamate (1.35 g, 2.98 mmol, 1.00 eq.) in DCM (15.0 mL) at 0° C., and the mixture was stirred at RT for 2 h. The mixture was quenched with H₂O and then extracted with DCM. The combined organic layer was washed with water, dried over anhydrous Na₂SO₄, filtered, and then concentrated. The residue was purified by silica gel column chromatography, eluted with PE/EA (1:1), to afford the title compound as white solid.

### Step 4: tert-Butyl (1-((3-(4-((4-(3-(2,4-dioxotetrahydropyrimidin-1(2H)-yl)-1-methyl-1H-indazol-6-yl)piperidin-1-yl)methyl)piperidin-1-yl)phenyl)sulfonyl)piperidin-4-yl)carbamate

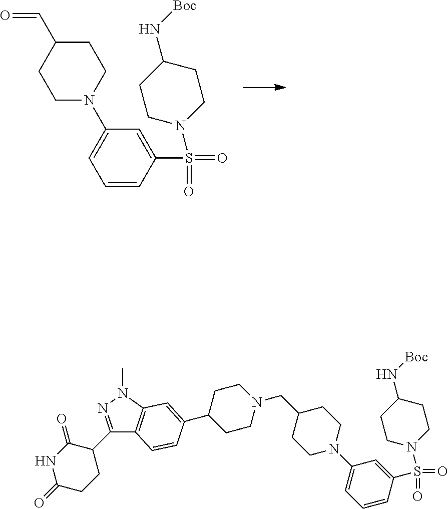 [SMILES: CC(C)(C)OC(=O)NC1CCN(S(=O)(=O)c2cccc(N3CCC(C=O)CC3)c2)CC1 ; Cn1nc(C2CCC(=O)NC2=O)c2ccc(C3CCN(CC4CCN(c5cccc(S(=O)(=O)N6CCC(NC(=O)OC(C)(C)C)CC6)c5)CC4)CC3)cc21]

A mixture of tert-butyl (1-((3-(4-formylpiperidin-1-yl)phenyl)sulfonyl)piperidin-4-yl)carbamate (264 mg, 0.585 mmol, 1.00 eq.), CH₃COOH (1 drops) and 1-(1-methyl-6-(piperidin-4-yl)-1H-indazol-3-yl)dihydropyrimidine-2,4(1H,3H)-dione (191.5 mg, 0.585 mmol, 1.00 eq.) in THF (2.0 mL)/DMF (2.0 mL) was stirred at 45° C. for 0.5 h. The mixture was cooled to 0° C. and NaBH₃CN (73.5 mg, 1.17 mmol, 2.00 eq.) was added. After stirring at RT for 12 h, the mixture was quenched with H₂O and extracted with DCM. The combined organic layer was washed with water, dried over anhydrous Na₂SO₄, filtered, and then concentrated. The residue was purified by silica gel column chromatography, eluted with DCM/MeOH=20:1 to give the title compound as yellow solid.

### Step 5: 1-(6-(1-((1-(3-((4-Aminopiperidin-1-yl)sulfonyl)phenyl)piperidin-4-yl)methyl)piperidin-4-yl)-1-methyl-1H-indazol-3-yl)dihydropyrimidine-2,4(1H,3H)-dione 2,2,2-trifluoroacetate

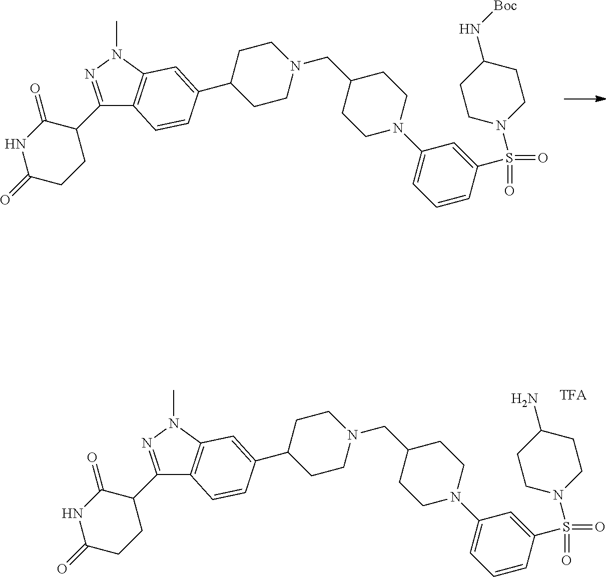 [SMILES: Cn1nc(C2CCC(=O)NC2=O)c2ccc(C3CCN(CC4CCN(c5cccc(S(=O)(=O)N6CCC(NC(=O)OC(C)(C)C)CC6)c5)CC4)CC3)cc21 ; Cn1nc(C2CCC(=O)NC2=O)c2ccc(C3CCN(CC4CCN(c5cccc(S(=O)(=O)N6CCC(N)CC6)c5)CC4)CC3)cc21]

A mixture of tert-butyl (1-((3-(4-((4-(3-(2,4-dioxotetrahydropyrimidin-1(2H)-yl)-1-methyl-1H-indazol-6-yl)piperidin-1-yl)methyl)piperidin-1-yl)phenyl)sulfonyl)piperidin-4-yl)carbamate (170 mg, 0.223 mmol, 1.00 eq.) in TFA/DCM (0.5 mL/2.0 mL) was stirred at rt for 2 h. The mixture was concentrated to give the title compound as yellow solid.

The following intermediates were prepared by proceeding analogously as described in Reference 56.

| Ref # | Structure | Synthesis |
|---|---|---|
| Ref. 56a | 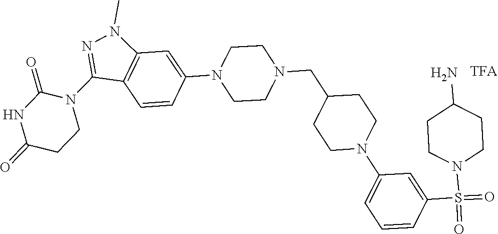 [SMILES: Cn1nc(N2CCC(=O)NC2=O)c2ccc(N3CCN(CC4CCN(c5cccc(S(=O)(=O)N6CCC(N)CC6)c5)CC4)CC3)cc21] | Ref 51 replaced 1-(1-methyl-6- (piperidin-4-yl)-1H-indazol-3- yl)dihydropyrimidine-2,4(1H, 3H)-dione in Step 4 |

### Reference 57

### Synthesis of tert-butyl 6-(3-(2,4-dioxotetrahydropyrimidin-1(2H)-yl)-1-methyl-1H-indazol-6-yl)-2,6-diazaspiro[3.3]heptane-2-carboxylate

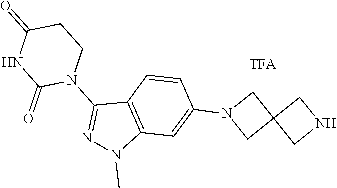 [SMILES: Cn1nc(N2CCC(=O)NC2=O)c2ccc(N3CC4(CNC4)C3)cc21]

### Step 1: tert-Butyl 6-(3-(2,4-dioxotetrahydropyrimidin-1(2H)-yl)-1-methyl-1H-indazol-6-yl)-2,6-diazaspiro[3.3]heptane-2-carboxylate

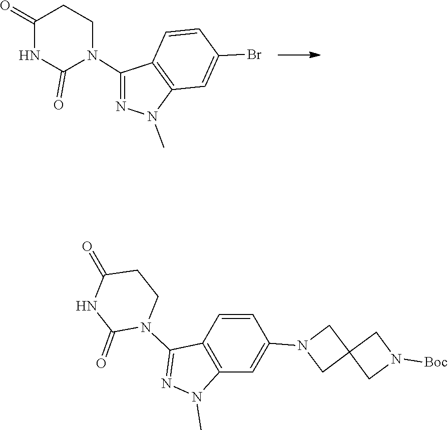 [SMILES: Cn1nc(N2CCC(=O)NC2=O)c2ccc(Br)cc21 ; Cn1nc(N2CCC(=O)NC2=O)c2ccc(N3CC4(CN(C(=O)OC(C)(C)C)C4)C3)cc21]

A mixture of 1-(6-bromo-1-methyl-1H-indazol-3-yl)dihydropyrimidine-2,4(1H,3H)-dione (300 mg, 0.93 mmol, 1.00 eq.), tert-butyl 2,6-diazaspiro[3.3]heptane-2-carboxylate (570 mg, 2.32 mmol, 2.50 eq.), t-BuOK (627 mg, 5.6 mmol, 6.00 eq.), t-BuBrettphos Pd G3 (81 mg, 0.093 mmol, 0.10 eq.) and t-BuXphos (76 mg, 0.186 mmol, 0.20 eq.) in 1,4-dioxane (6 mL) was stirred at 100° C. under N₂ for 3 h. The mixture was diluted with DCM and the organic layer was washed with water and brine, dried over Na₂SO₄, and concentrated. Purification of the residue by column chromatography on silica gel (DCM:MeOH=20:1) to give the title compound as yellow solid.

### Step 2: 1-(1-Methyl-6-(2,6-diazaspiro[3.3]heptan-2-yl)-1H-indazol-3-yl)dihydropyrimidine-2,4(1H,3H)-dione 2,2,2-trifluoroacetate

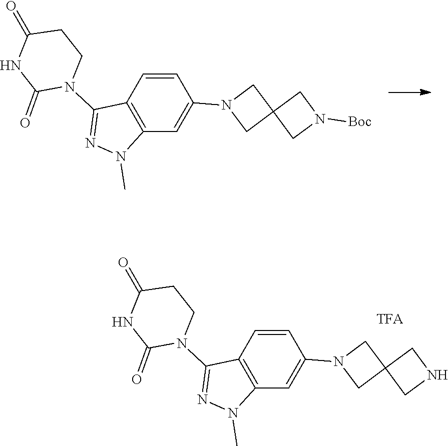 [SMILES: Cn1nc(N2CCC(=O)NC2=O)c2ccc(N3CC4(CN(C(=O)OC(C)(C)C)C4)C3)cc21 ; Cn1nc(N2CCC(=O)NC2=O)c2ccc(N3CC4(CNC4)C3)cc21]

A mixture of tert-butyl 6-(3-(2,4-dioxotetrahydropyrimidin-1(2H)-yl)-1-methyl-1H-indazol-6-yl)-2,6-diazaspiro[3.3]heptane-2-carboxylate (90 mg, 0.204 mmol, 1.00 eq.) in TFA/DCM (0.5 mL/2 mL) was stirred at rt for 2 h. The mixture was concentrated to give the title compound as brown oil.

### Reference 58

### Synthesis of 1-(6-(1-(3-(((1s,4s)-4-aminocyclohexyl)sulfonyl)benzyl)piperidin-4-yl)-1-methyl-1H-indazol-3-yl)dihydropyrimidine-2,4(1H,3H)-dione 2,2,2-trifluoroacetate

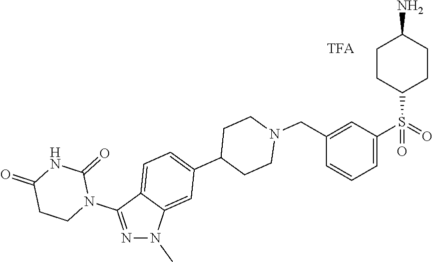

### Step 1:(3-Mercaptophenyl) methanol

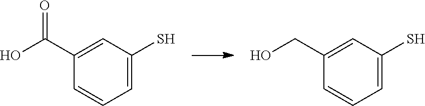 [SMILES: O=C(O)c1cccc(S)c1 ; OCc1cccc(S)c1]

LiAlH₄ (38.92 mL, 38.92 mmol, 3.0 eq) was added to a suspension of 3-sulfanylbenzoic acid (2 g, 12.97 mmol, 1.0 eq) in anhydrous THF (50 mL). The reaction mixture was stirred at rt for 1 h and then refluxed for 6 h. The suspension was allowed to cool to rt and stirred overnight before being quenched at 0° C. by the slow addition of water, 1N aq. NaOH and water. The solid was removed by filtration. The solid was dissolved in 1N aq. HCl and the resulting solution was extracted with EA. The combined organic extracts were concentrated under reduced pressure. The residue was purified by silica gel column chromatography to afford the title compound as a yellow oil.

### Step 2: tert-Butyl ((1r,4r)-4-((3-(hydroxymethyl)phenyl)thio)cyclohexyl)carbamate

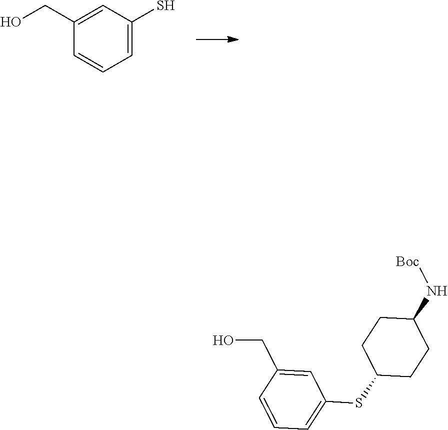 [SMILES: OCc1cccc(S)c1 ; CC(C)(C)OC(=O)N[C@H]1CC[C@H](Sc2cccc(CO)c2)CC1]

A mixture of [4-(tert-butoxycarbonylamino)cyclohexyl] 4-methylbenzenesulfonate (2.9 g, 7.85 mmol, 1.0 eq) and (3-mercaptophenyl) methanol (1.1 g, 7.85 mmol, 1.0 eq), potassium carbonate (2.17 g, 15.7 mmol, 2.0 eq) in MeCN (100 mL) was degassed and refilled with N₂ and stirred at 80° C. for 16 h. The mixture was filtered through Celite and the filter cake was washed with MeCN. The combined organic layers were concentrated under reduced pressure and the residue was purified by silica gel column chromatography, eluted with EA/PE (0-25%) to afford the title compound as a white solid.

### Step 3: tert-Butyl ((1r,4r)-4-((3-formylphenyl)thio)cyclohexyl)carbamate

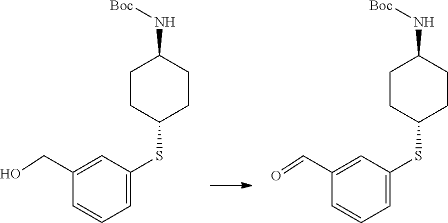 [SMILES: CC(C)(C)OC(=O)N[C@H]1CC[C@H](Sc2cccc(CO)c2)CC1 ; CC(C)(C)OC(=O)N[C@H]1CC[C@H](Sc2cccc(C=O)c2)CC1]

To a stirred mixture of tert-butyl ((1r,4r)-4-((3-(hydroxymethyl)phenyl)thio)cyclohexyl)-carbamate (260 mg, 0.77 mmol, 1.0 eq) in anhydrous DCM (15 mL) at 0° C., was added DMP (653.5 mg, 1.54 mmol, 2.0 eq) in portions. The resulting mixture was stirred for 2 h at 25° C. The resulting mixture was diluted with water, quenched with saturated Na₂S₂O₃ and saturated NaHCO₃ at 0° C. The resulting mixture was stirred at rt for 10 min and extracted with DCM, and dried over anhydrous Na₂SO₄. After filtration, the filtrate was concentrated under reduced pressure and the residue was purified by silica gel column chromatography, eluted with EA/PE (0-25%) to afford the title compound as a white solid.

### Step 4: tert-Butyl ((1r,4r)-4-((3-((4-(3-(2,4-dioxotetrahydropyrimidin-1(2H)-yl)-1-methyl-1H-indazol-6-yl)piperidin-1-yl)methyl)phenyl)thio)cyclohexyl)carbamate

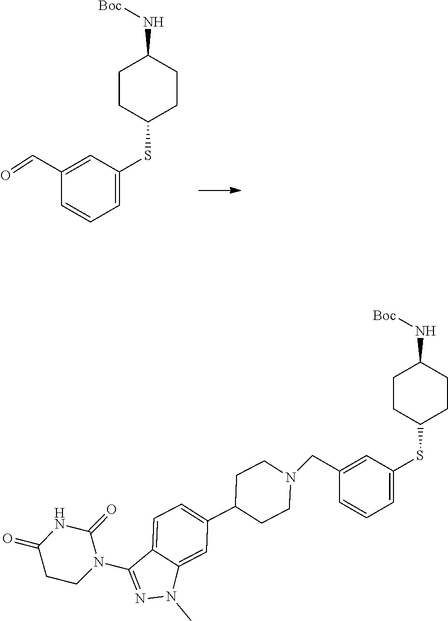

To a stirred mixture of tert-butyl ((1r,4r)-4-((3-formylphenyl)thio)cyclohexyl)carbamate (200 mg, 0.6 mmol, 1.0 eq) and 1-[1-methyl-6-(4-piperidyl) indazol-3-yl]hexahydropyrimidine-2,4-dione (hydrochloride salt, 216.9 mg, 0.6 mmol, 1.0 eq) in anhydrous DCE (20 mL) at 0° C. was added sodium triacetoxyborohydride (379.1 mg, 1.8 mmol, 1.0 eq) in portions and the resulting mixture was stirred for 16 h at 25° C. The mixture was diluted with water, quenched with saturated NaHCO₃ at 0° C. and stirred at rt for 10 min. The mixture was extracted with DCM, and the organic layer was washed with brine, dried over anhydrous Na₂SO₄. After filtration, the filtrate was concentrated under reduced pressure and the residue was purified by silica gel column chromatography, eluted with MeOH/DCM (0-5%) to afford title compound as a white solid.

### Step 5: 1-(6-(1-(3-(((1r,4r)-4-Aminocyclohexyl)thio)benzyl)piperidin-4-yl)-1-methyl-1H-indazol-3-yl)dihydropyrimidine-2,4(1H,3H)-dione 2,2,2-trifluoroacetate

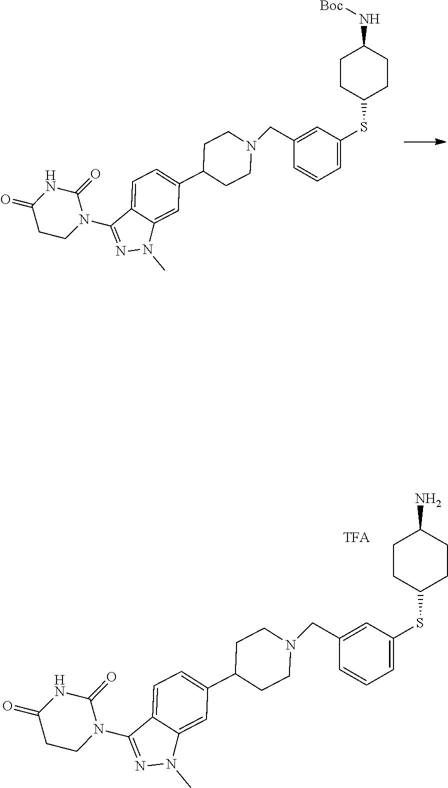

To a stirred mixture of tert-butyl ((1r,4r)-4-((3-((4-(3-(2,4-dioxotetrahydropyrimidin-1(2H)-yl)-1-methyl-1H-indazol-6-yl)piperidin-1-yl)methyl)phenyl)thio)cyclohexyl)carbamate (100 mg, 0.15 mmol, 1.0 eq) in anhydrous DCM (10 mL) at 0° C. was added TFA (0.24 mL) dropwise. The resulting mixture was stirred for 2 h at 25° C. and was concentrated under reduced pressure to afford the title compound as a yellow oil.

### Step 6: 1-(6-(1-(3-(((1s,4s)-4-Aminocyclohexyl)sulfonyl)benzyl)piperidin-4-yl)-1-methyl-1H-indazol-3-yl)dihydropyrimidine-2,4(1H,3H)-dione 2,2,2-trifluoroacetate

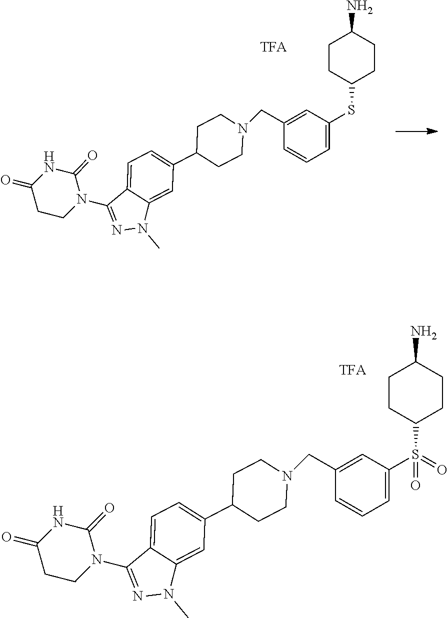

To a stirred mixture of 1-(6-(1-(3-(((1r,4r)-4-aminocyclohexyl)thio)benzyl)piperidin-4-yl)-1-methyl-1H-indazol-3-yl)dihydro pyrimidine-2,4(1H,3H)-dione 2,2,2-trifluoroacetate (0.15 mmol, 1.0 eq) in anhydrous DCM (10 mL) at 0° C. was added m-CPBA (78.4 mg, 0.45 mmol, 3.0 eq) and the resulting mixture was stirred for 16 h at 25° C. The mixture was concentrated under reduced pressure to afford the title compound as a white solid.

### Reference 59

### Synthesis of (2S,4R)-1-((R)-3-(((1-(3-((4-aminopiperidin-1-yl)sulfonyl)benzyl)piperidin-4-yl)-methyl)thio)-2-(1-fluorocyclopropane-1-carboxamido)-3-methylbutanoyl)-4-hydroxy-N—((S)-1-(4-(4-methylthiazol-5-yl)phenyl)ethyl) pyrrolidine-2-carboxamide

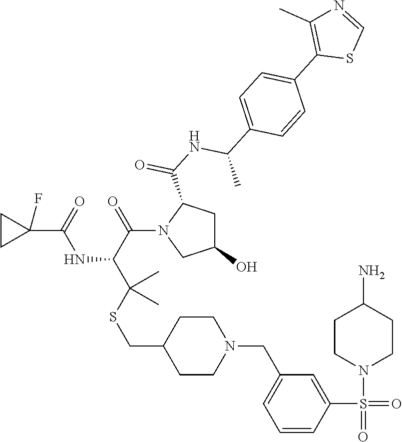 [SMILES: Cc1ncsc1-c1ccc([C@H](C)NC(=O)[C@@H]2C[C@@H](O)CN2C(=O)[C@@H](NC(=O)C2(F)CC2)C(C)(C)SCC2CCN(Cc3cccc(S(=O)(=O)N4CCC(N)CC4)c3)CC2)cc1]

### Step 1: tert-Butyl(2S,4R)-4-hydroxy-2-(((S)-1-(4-(4-methylthiazol-5-yl)phenyl)ethyl) carbamoyl) pyrrolidine-1-carboxylate

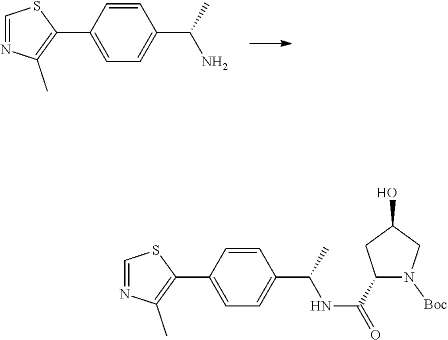 [SMILES: Cc1ncsc1-c1ccc([C@H](C)N)cc1 ; Cc1ncsc1-c1ccc([C@H](C)NC(=O)[C@@H]2C[C@@H](O)CN2C(=O)OC(C)(C)C)cc1]

A mixture of (1S)-1-[4-(4-methylthiazol-5-yl)phenyl]ethanamine (2480. mg, 11.36 mmol) and (2S,4R)-1-tert-butoxycarbonyl-4-hydroxy-pyrrolidine-2-carboxylic acid (2889.57 mg, 12.5 mmol), HATU (5183.19 mg, 13.63 mmol) and TEA (7.92 mL, 56.8 mmol) in DCM (25 mL) was stirred at 25° C. for 2 h. The mixture was diluted with water and extracted with DCM, and the organic phase was dried over anhydrous sodium sulfate and concentrated. The residue was purified by flash column chromatography to give the title compound.

### Step 2: (2S,4R)-4-Hydroxy-N—((S)-1-(4-(4-methylthiazol-5-yl)phenyl)ethyl) pyrrolidine-2-carboxamide

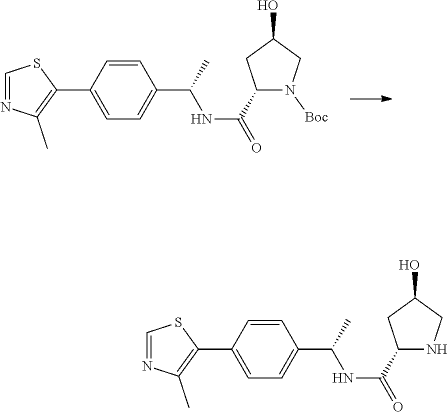 [SMILES: Cc1ncsc1-c1ccc([C@H](C)NC(=O)[C@@H]2C[C@@H](O)CN2C(=O)OC(C)(C)C)cc1 ; Cc1ncsc1-c1ccc([C@H](C)NC(=O)[C@@H]2C[C@@H](O)CN2)cc1]

To a solution of tert-butyl(2S,4R)-4-hydroxy-2-[[(1S)-1-[4-(4-methylthiazol-5-yl)-phenyl]ethyl]carbamoyl]pyrrolidine-1-carboxylate (3.77 g, 8.74 mmol) in DCM (20 mL) was added 4M HCl-dioxane (20 mL, 80 mmol) and the reaction mixture was stirred at room temperature for 1 h. The solvent was evaporated under reduced pressure to afford the title compound.

### Step 3: (9H-Fluoren-9-yl)methyl ((R)-1-((2S,4R)-4-hydroxy-2-(((S)-1-(4-(4-methylthiazol-5-yl)phenyl)ethyl) carbamoyl) pyrrolidin-1-yl)-3-methyl-1-oxo-3-(tritylthio) butan-2-yl)carbamate

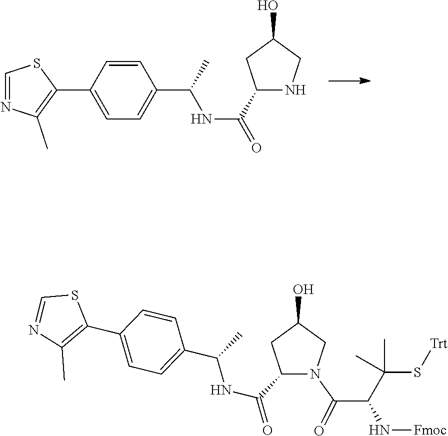

To a solution of (2R)-2-(9H-fluoren-9-ylmethoxycarbonylamino)-3-methyl-3-trityl-sulfanyl-butanoic acid (1576.6 mg, 2.57 mmol), (2S,4R)-4-hydroxy-N-[(1S)-1-[4-(4-methyl-thiazol-5-yl)phenyl]ethyl]pyrrolidine-2-carboxamide hydrochloride (1350 mg, 3.67 mmmol) in DMF (10 mL) was added TEA (1.53 mL, 11.01 mmol), and HATU (2790.6 mg, 7.34 mmol), and the mixture was stirred at room temperature for 2 h. The reaction mixture was diluted with water, extracted with EA. The combined organic layers were washed with brine, dried over anhydrous sodium sulfate, filtered, and the filtrate was concentrated under reduced pressure. The residue was purified by flash silica gel column chromatography to afford the title compound.

### Step 4: (2S,4R)-1-((R)-2-Amino-3-methyl-3-(tritylthio) butanoyl)-4-hydroxy-N—((S)-1-(4-(4-methylthiazol-5-yl)phenyl)ethyl) pyrrolidine-2-carboxamide

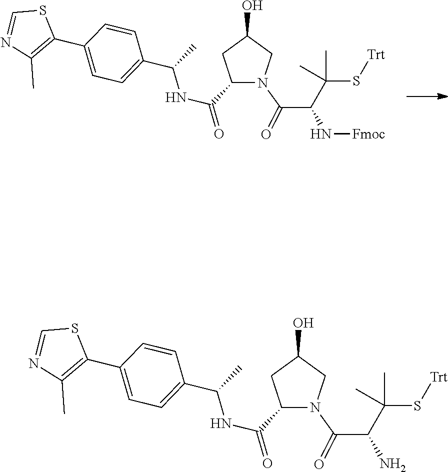

To a solution of 9H-fluoren-9-ylmethyl N-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-2-tritylsulfanyl-propyl]carbamate (1360 mg, 1.47 mmol) in DCM (10 mL) was added piperidine (0.29 mL, 2.93 mmol) at room temperature, and the solution was stirred at 25° C. for 3 h. The reaction mixture was diluted with water, extracted with EA. The combined organic layers were washed with brine, dried over anhydrous sodium sulfate, filtered, and the filtrate was concentrated under reduced pressure. The residue was purified by flash silica gel column chromatography to afford the title compound.

### Step 5: (2S,4R)-1-((R)-2-(1-Fluorocyclopropane-1-carboxamido)-3-methyl-3-(tritylthio)-butanoyl)-4-hydroxy-N—((S)-1-(4-(4-methylthiazol-5-yl)phenyl)ethyl) pyrrolidine-2-carboxamide

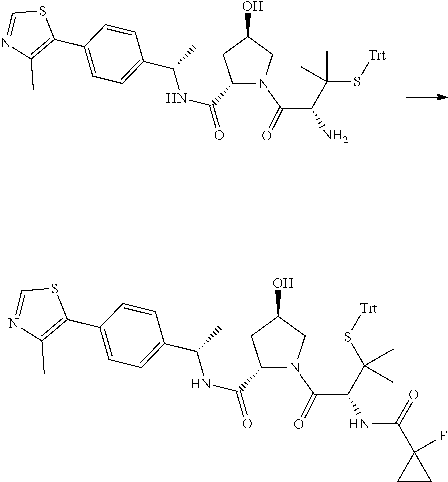

To a solution of (2S,4R)-1-[(2R)-2-amino-3-methyl-3-tritylsulfanyl-butanoyl]-4-hydroxy-N-[(1S)-1-[4-(4-methylthiazol-5-yl)phenyl]ethyl]pyrrolidine-2-carboxamide (2.1 g, 2.98 mmol) and HATU (2265.4 mg, 5.96 mmol) in DCM (1 mL) was added TEA (1155.0 mg, 8.94 mmol). The reaction mixture was purged with Argon and then HATU (2265.4 mg, 5.96 mmol) was added, an the reaction mixture was stirred at room temperature for 1 h. The mixture was diluted with water and extracted with DCM, and the organic phase was dried over anhydrous sodium sulfate. The organic layer was concentrated to afford the title compound.

### Step 6: (2S,4R)-1-((R)-2-(1-fluorocyclopropane-1-carboxamido)-3-mercapto-3-methylbutanoyl)-4-hydroxy-N—((S)-1-(4-(4-methylthiazol-5-yl)phenyl)ethyl) pyrrolidine-2-carboxamide

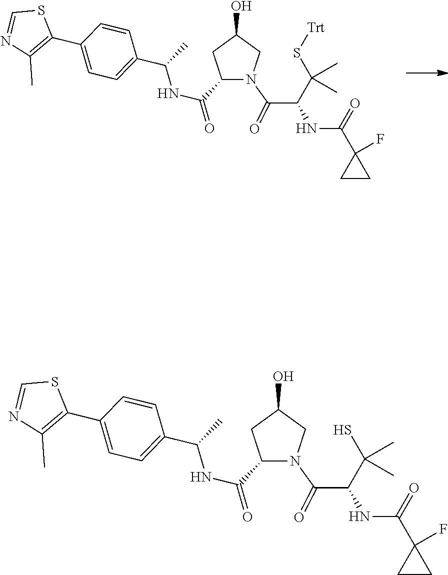

To a solution of (2S,4R)-1-((R)-2-(1-fluorocyclopropane-1-carboxamido)-3-methyl-3-(tritylthio) butanoyl)-4-hydroxy-N—((S)-1-(4-(4-methylthiazol-5-yl)phenyl)ethyl) pyrrolidine-2-carboxamide (1.2 g, 1.52 mmol) in DCM (10 mL) was added TFA (10 mL, 129.8 mmol) and triisopropylsilane (1.03 mL, 5.01 mmol). The reaction mixture was stirred at room temperature for 2 h and then diluted with water and extracted with DCM. The organic phase was dried over anhydrous sodium sulfate and concentrated to afford the title compound.

### Step 7: tert-Butyl 4-((((R)-3-(1-fluorocyclopropane-1-carboxamido)-4-((2S,4R)-4-hydroxy-2-(((S)-1-(4-(4-methylthiazol-5-yl)phenyl)ethyl) carbamoyl) pyrrolidin-1-yl)-2-methyl-4-oxobutan-2-yl)thio)methyl)piperidine-1-carboxylate

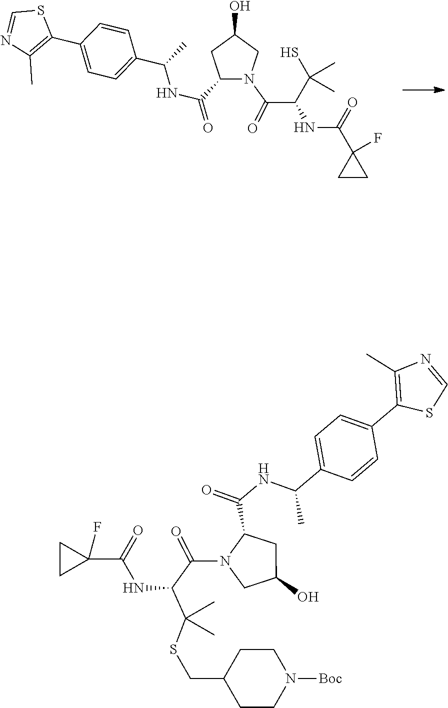 [SMILES: Cc1ncsc1-c1ccc([C@H](C)NC(=O)[C@@H]2C[C@@H](O)CN2C(=O)[C@@H](NC(=O)C2(F)CC2)C(C)(C)S)cc1 ; Cc1ncsc1-c1ccc([C@H](C)NC(=O)[C@@H]2C[C@@H](O)CN2C(=O)[C@@H](NC(=O)C2(F)CC2)C(C)(C)SCC2CCN(C(=O)OC(C)(C)C)CC2)cc1]

DBU (2433.9 mg, 16.0 mmol) was added to a solution of (2R,4R)-1-[(2R)-2-[(1-fluorocyclopropanecarbonyl)amino]-3-methyl-3-sulfanyl-butanoyl]-4-hydroxy-N-[(1S)-1-[4-(4-methylthiazol-5-yl)phenyl] ethyl]pyrrolidine-2-carboxamide (1.7 g, 2.66 mmol) and tert-butyl 4-(bromomethyl)piperidine-1-carboxylate (1.04 mg, 3.73 mmol) in THF (20 mL) at room temperature, and the reaction was stirred at room temperature overnight. The mixture was diluted with water and extracted with EA, and the organic phase was dried over anhydrous sodium sulfate and concentrated. The residue was purified by flash column chromatography to obtain the title compound.

### Step 8: (2S,4R)-1-((R)-2-(1-fluorocyclopropane-1-carboxamido)-3-methyl-3-((piperidin-4-yl-methyl)thio) butanoyl)-4-hydroxy-N—((S)-1-(4-(4-methylthiazol-5-yl)phenyl)ethyl) pyrrolidine-2-carboxamide

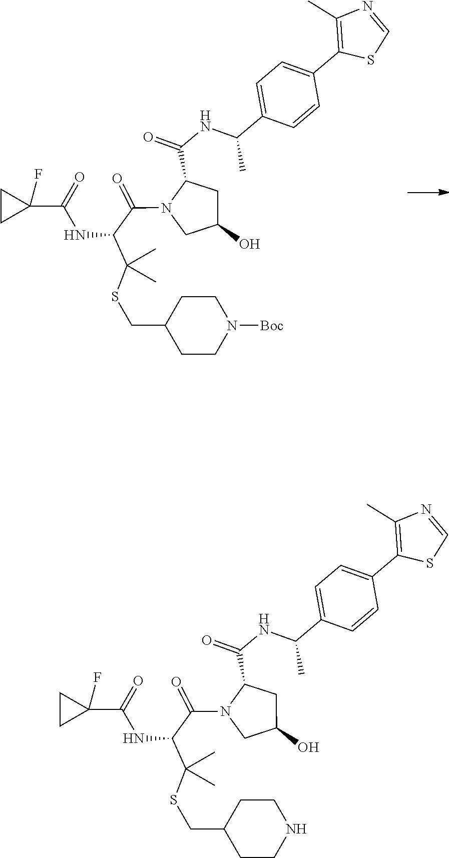 [SMILES: Cc1ncsc1-c1ccc([C@H](C)NC(=O)[C@@H]2C[C@@H](O)CN2C(=O)[C@@H](NC(=O)C2(F)CC2)C(C)(C)SCC2CCN(C(=O)OC(C)(C)C)CC2)cc1 ; Cc1ncsc1-c1ccc([C@H](C)NC(=O)[C@@H]2C[C@@H](O)CN2C(=O)[C@@H](NC(=O)C2(F)CC2)C(C)(C)SCC2CCNCC2)cc1]

To a solution of tert-butyl 4-[[(2R)-2-[(1-fluorocyclopropanecarbonyl)amino]-3-[(2S,4R)-4-hydroxy-2-[[(1S)-1-[4-(4-methylthiazol-5-yl)phenyl]ethyl]carbamoyl]pyrrolidin-1-yl]-1,1-dimethyl-3-oxo-propyl]sulfanylmethyl]piperidine-1-carboxylate (1.1 g, 1.47 mmol) in DCM (10 mL) was added 4M HCl-Dioxane (5. mL, 1.47 mmol) at room temperature. The reaction mixture was stirred at room temperature for 1 h and then concentrated to afford the title compound.

### Step 9: tert-Butyl (1-((3-((4-((((R)-3-(1-fluorocyclopropane-1-carboxamido)-4-((2S,4R)-4-hydroxy-2-(((S)-1-(4-(4-methylthiazol-5-yl)phenyl)ethyl) carbamoyl) pyrrolidin-1-yl)-2-methyl-4-oxobutan-2-yl)thio)methyl)piperidin-1-yl)methyl)phenyl)sulfonyl)piperidin-4-yl)carbamate

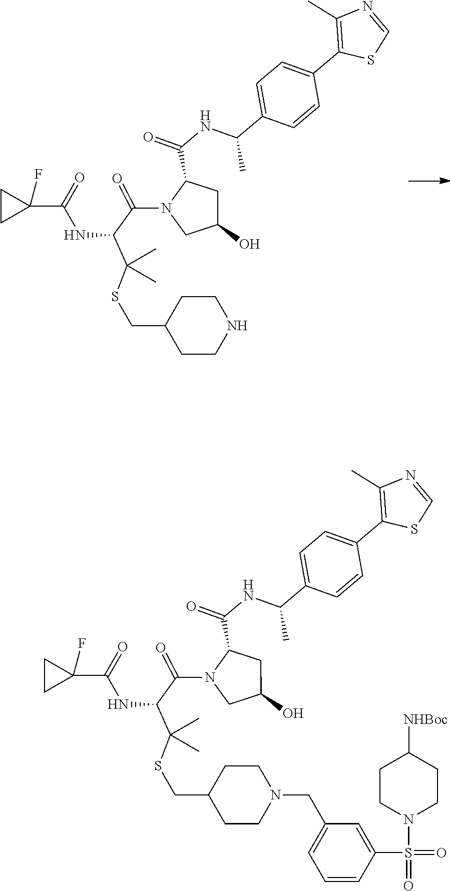 [SMILES: Cc1ncsc1-c1ccc([C@H](C)NC(=O)[C@@H]2C[C@@H](O)CN2C(=O)[C@@H](NC(=O)C2(F)CC2)C(C)(C)SCC2CCNCC2)cc1 ; Cc1ncsc1-c1ccc([C@H](C)NC(=O)[C@@H]2C[C@@H](O)CN2C(=O)[C@@H](NC(=O)C2(F)CC2)C(C)(C)SCC2CCN(Cc3cccc(S(=O)(=O)N4CCC(NC(=O)OC(C)(C)C)CC4)c3)CC2)cc1]

A mixture of (2S,4R)-1-((R)-2-(1-fluorocyclopropane-1-carboxamido)-3-methyl-3-((piperidin-4-ylmethyl)thio) butanoyl)-4-hydroxy-N—((S)-1-(4-(4-methylthiazol-5-yl)phenyl)-ethyl) pyrrolidine-2-carboxamide (300 mg, 0.46 mmol) and tert-butyl N-[1-[3-(bromomethyl)-phenyl]sulfonyl-4-piperidyl]carbamate (221.43 mg, 0.51 mmol), TEA (0.39 mL, 2.79 mmol) in DMF (2 mL) was stirred at 55° C. for 3 h. The mixture was cooled to room temperature and diluted with water and extracted with EA. The organic phase was dried over anhydrous sodium sulfate and concentrated and the residue was purified by flash column chromatography to give the title compound.

### Step 10: (2S,4R)-1-((R)-3-(((1-(3-((4-aminopiperidin-1-yl)sulfonyl)benzyl)piperidin-4-yl)methyl)-thio)-2-(1-fluorocyclopropane-1-carboxamido)-3-methylbutanoyl)-4-hydroxy-N—((S)-1-(4-(4-methylthiazol-5-yl)phenyl)ethyl) pyrrolidine-2-carboxamide

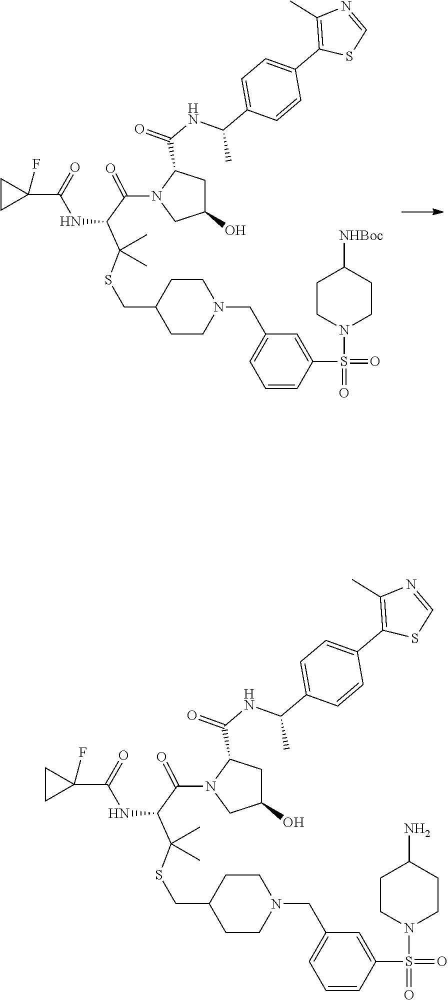 [SMILES: Cc1ncsc1-c1ccc([C@H](C)NC(=O)[C@@H]2C[C@@H](O)CN2C(=O)[C@@H](NC(=O)C2(F)CC2)C(C)(C)SCC2CCN(Cc3cccc(S(=O)(=O)N4CCC(NC(=O)OC(C)(C)C)CC4)c3)CC2)cc1 ; Cc1ncsc1-c1ccc([C@H](C)NC(=O)[C@@H]2C[C@@H](O)CN2C(=O)[C@@H](NC(=O)C2(F)CC2)C(C)(C)SCC2CCN(Cc3cccc(S(=O)(=O)N4CCC(N)CC4)c3)CC2)cc1]

To a solution of tert-butyl (1-((3-((4-((((R)-3-(1-fluorocyclopropane-1-carboxamido)-4-((2S,4R)-4-hydroxy-2-(((S)-1-(4-(4-methylthiazol-5-yl)phenyl)ethyl) carbamoyl) pyrrolidin-1-yl)-2-methyl-4-oxobutan-2-yl)thio)methyl)piperidin-1-yl)methyl)phenyl)sulfonyl)piperidin-4-yl)-carbamate (450 mg, 0.45 mmol) in DCM (4 mL) was added 4M HCl-Dioxane (2 mL, 0.45 mmol) at 25° C., and the mixture was stirred at 25° C. for 1 h. The mixture was concentrated under reduced pressure to afford the title compound.

### Reference 60

### Synthesis of 1-(6-(1-(3-((4-aminopiperidin-1-yl)sulfonyl)phenethyl)piperidin-4-yl)-1-methyl-1H-indazol-3-yl)dihydropyrimidine-2,4(1H,3H)-dione hydrochloride

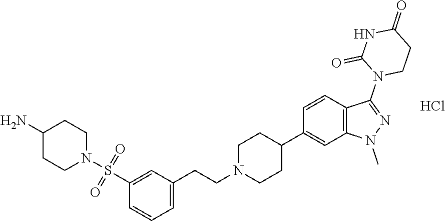 [SMILES: Cn1nc(N2CCC(=O)NC2=O)c2ccc(C3CCN(CCc4cccc(S(=O)(=O)N5CCC(N)CC5)c4)CC3)cc21 ; Cl]

### Step 1: 1-(1-Methyl-6-(piperidin-4-yl)-1H-indazol-3-yl)dihydropyrimidine-2,4(1H,3H)-dione hydrochloride

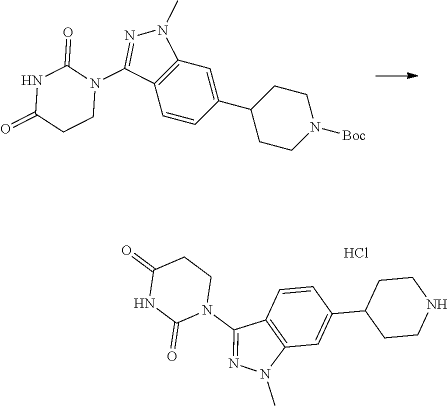 [SMILES: Cn1nc(N2CCC(=O)NC2=O)c2ccc(C3CCN(C(=O)OC(C)(C)C)CC3)cc21 ; Cn1nc(N2CCC(=O)NC2=O)c2ccc(C3CCNCC3)cc21 ; Cl]

To a stirred solution of tert-butyl 4-(3-(2,4-dioxotetrahydropyrimidin-1(2H)-yl)-1-methyl-1H-indazol-6-yl)piperidine-1-carboxylate (7.2 g, 16.84 mmol) in dichloromethane (72 mL) was added 4 M HCl in 1,4-dioxane (36 mL) at 0° C. and stirred for 1 h. The mixture was concentrated to afford the title compound as an off-white solid.

### Step 2: tert-Butyl (1-((3-vinylphenyl)sulfonyl)piperidin-4-yl)carbamate

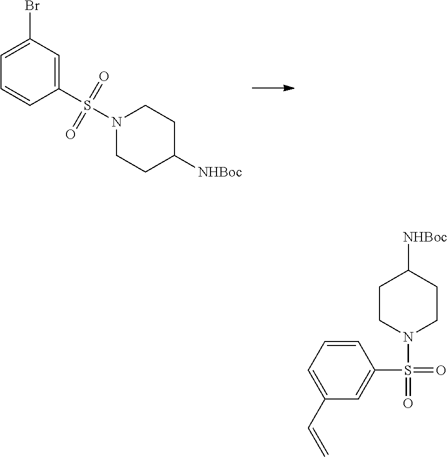 [SMILES: CC(C)(C)OC(=O)NC1CCN(S(=O)(=O)c2cccc(Br)c2)CC1 ; C=Cc1cccc(S(=O)(=O)N2CCC(NC(=O)OC(C)(C)C)CC2)c1]

A mixture of tert-butyl (1-((3-bromophenyl)sulfonyl)piperidin-4-yl)carbamate (5 g, 11.92 mmol), potassium vinyltrifluoroborate (2.4 g, 17.89 mmol), Pd(dppf)Cl₂ (872.48 mg, 1.19 mmol) and potassium carbonate (4.9 g, 35.77 mmol) in dioxane/acetonitrile/water (60 mL, 5:5:2, v/v) was purged with argon five times and stirred at 85° C. 6 h. The mixture was diluted with ethyl acetate and filtered. The filtrate was washed with water, brine, dried over anhydrous sodium sulfate, filtered and concentrated. The residue was purified by silica gel chromatography, eluted with ethyl acetate/petroleum ether (0˜50%) to afford the title compound as a yellow solid.

### Step 3: tert-Butyl (1-((3-(2-hydroxyethyl)phenyl)sulfonyl)piperidin-4-yl)carbamate

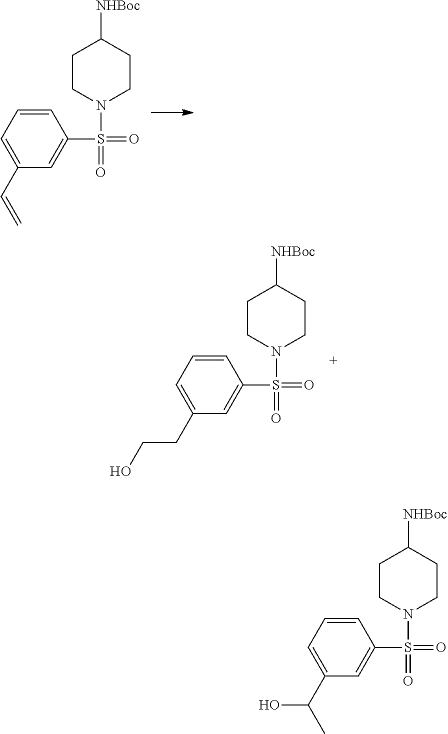 [SMILES: C=Cc1cccc(S(=O)(=O)N2CCC(NC(=O)OC(C)(C)C)CC2)c1 ; CC(C)(C)OC(=O)NC1CCN(S(=O)(=O)c2cccc(CCO)c2)CC1 ; CC(O)c1cccc(S(=O)(=O)N2CCC(NC(=O)OC(C)(C)C)CC2)c1]

To a stirred solution of tert-butyl (1-((3-vinylphenyl)sulfonyl)piperidin-4-yl)carbamate (4.05 g, 11.05 mmol) in anhydrous tetrahydrofuran (40 mL) was added borane-tetrahydrofuran complex (16.58 mL, 16.58 mmol) dropwise at 25° C. under argon atmosphere and the mixture was stirred for 2.5 h. 10% sodium hydroxide aq (8840.86 mg, 22.1 mmol) was added slowly, followed by hydrogen peroxide (2.26 mL, 22.1 mmol, 30%). The resulting mixture was stirred at 25° C. for 3.5 h. The reaction mixture was quenched with ammonium chloride (aq.) and extracted with ethyl acetate. The combined organic layers were washed with water, brine, dried over anhydrous sodium sulfate. After filtration, the filtrated was concentrated under reduced pressure and the residue was purified by silica gel chromatography, eluted with ethyl acetate/petroleum ether (0˜50%) to afford the title compound and its isomer as white solid.

### Step 4: tert-Butyl (1-((3-(2-oxoethyl)phenyl)sulfonyl)piperidin-4-yl)carbamate

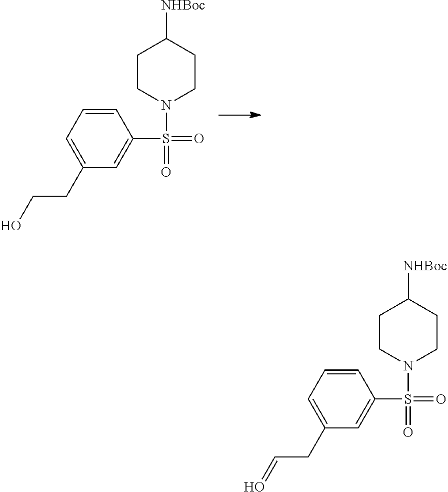 [SMILES: CC(C)(C)OC(=O)NC1CCN(S(=O)(=O)c2cccc(CCO)c2)CC1 ; CC(C)(C)OC(=O)NC1CCN(S(=O)(=O)c2cccc(CC=O)c2)CC1]

To a stirred solution of tert-butyl (1-((3-(2-hydroxyethyl)phenyl)sulfonyl)piperidin-4-yl)-carbamate (200 mg, 0.52 mmol) in anhydrous dichloromethane (5 mL) was added Dess-Martin Periodinane (441.25 mg, 1.04 mmol) at 0° C. and the mixture was stirred for 1 h. The mixture was diluted with ethyl acetate, washed with sodium sulfite (aq.), sodium bicarbonate (aq.), water, brine, and the organic layer was dried over anhydrous sodium sulfate. After filtration, the filtrate was concentrated under reduced pressure to afford the title compound as a white solid.

### Step 5: tert-Butyl (1-((3-(2-(4-(3-(2,4-dioxotetrahydropyrimidin-1(2H)-yl)-1-methyl-1H-indazol-6-yl)piperidin-1-yl)ethyl)phenyl)sulfonyl)piperidin-4-yl)carbamate

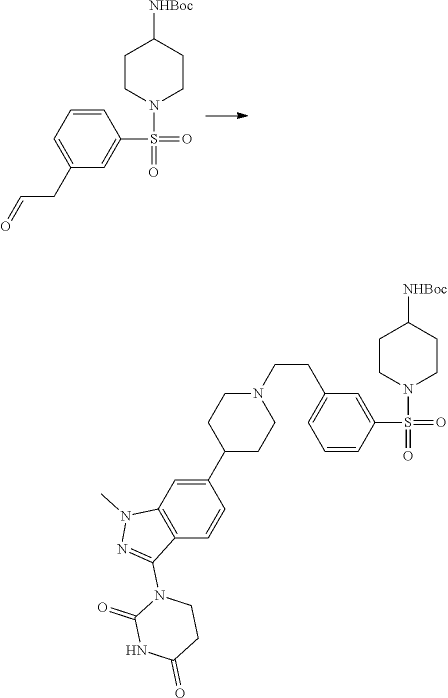 [SMILES: CC(C)(C)OC(=O)NC1CCN(S(=O)(=O)c2cccc(CC=O)c2)CC1 ; Cn1nc(N2CCC(=O)NC2=O)c2ccc(C3CCN(CCc4cccc(S(=O)(=O)N5CCC(NC(=O)OC(C)(C)C)CC5)c4)CC3)cc21]

A mixture of 1-(1-methyl-6-(piperidin-4-yl)-1H-indazol-3-yl)dihydropyrimidine-2,4(1H,3H)-dione hydrochloride (95.13 mg, 0.26 mmol) and tert-butyl (1-((3-(2-oxoethyl)-phenyl)sulfonyl)piperidin-4-yl)carbamate (100 mg, 0.26 mmol) in anhydrous dichloromethane (2 mL) was stirred at 25° C. for 1 h. Sodium triacetoxyborohydride (110.82 mg, 0.52 mmol) was added and the mixture was stirred for 2 h. The reaction mixture was diluted with water and extracted with ethyl acetate and the combined organic layers were washed with water, brine, and dried over anhydrous sodium sulfate. After filtration, the filtrate was concentrated under reduced pressure and the residue was purified by silica gel chromatography, eluted with methanol/dichloromethane (0˜5%) to afford title compound as a yellow solid.

### Step 6: 1-(6-(1-(3-((4-Aminopiperidin-1-yl)sulfonyl)phenethyl)piperidin-4-yl)-1-methyl-1H-indazol-3-yl)dihydropyrimidine-2,4(1H,3H)-dione hydrochloride

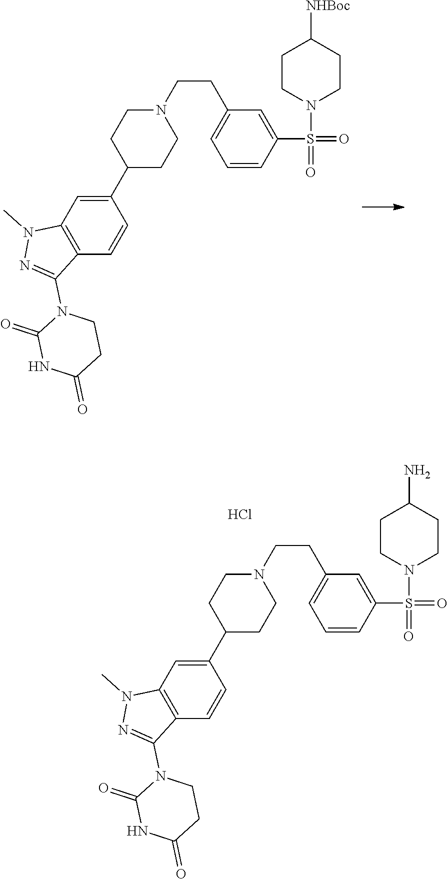 [SMILES: Cn1nc(N2CCC(=O)NC2=O)c2ccc(C3CCN(CCc4cccc(S(=O)(=O)N5CCC(NC(=O)OC(C)(C)C)CC5)c4)CC3)cc21 ; Cn1nc(N2CCC(=O)NC2=O)c2ccc(C3CCN(CCc4cccc(S(=O)(=O)N5CCC(N)CC5)c4)CC3)cc21 ; Cl]

To a stirred solution of tert-butyl (1-((3-(2-(4-(3-(2,4-dioxotetrahydropyrimidin-1(2H)-yl)-1-methyl-1H-indazol-6-yl)piperidin-1-yl)ethyl)phenyl)sulfonyl)piperidin-4-yl)carbamate (100 mg, 0.14 mmol) in dichloromethane (3 mL) was added 4 M hydrogen chloride/1,4-dioxane (1.5 mL) at 0° C. and stirred for 1 h. The solvent was removed under reduced pressure to afford the title compound as a white solid.

### Reference 61

### Synthesis of 1-(6-(1-(3-(3-((4-Aminopiperidin-1-yl)sulfonyl)phenyl)-2-methylpropyl)piperidin-4-yl)-1-methyl-1H-indazol-3-yl)dihydropyrimidine-2,4(1H,3H)-dione hydrochloride

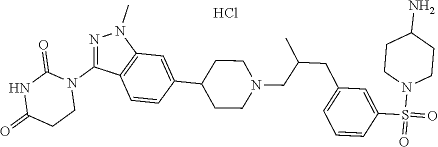 [SMILES: CC(Cc1cccc(S(=O)(=O)N2CCC(N)CC2)c1)CN1CCC(c2ccc3c(N4CCC(=O)NC4=O)nn(C)c3c2)CC1 ; Cl]

### Step 1: tert-Butyl (1-((3-(2-methyl-3-oxopropyl)phenyl)sulfonyl)piperidin-4-yl)carbamate

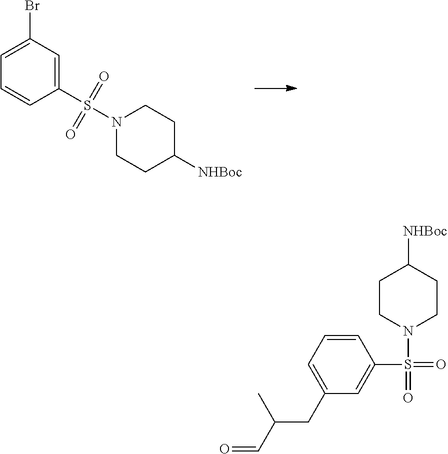 [SMILES: CC(C)(C)OC(=O)NC1CCN(S(=O)(=O)c2cccc(Br)c2)CC1 ; CC(C=O)Cc1cccc(S(=O)(=O)N2CCC(NC(=O)OC(C)(C)C)CC2)c1]

A mixture of palladium (II) acetate (53.54 mg, 0.24 mmol) and tetrabutylammonium bromide (4.0 g, 12.41 mmol) was heated to 130° C. under argon atmosphere, and then tert-butyl (1-((3-bromophenyl)sulfonyl)piperidin-4-yl)carbamate (1.0 g, 2.38 mmol), 2-methylprop-2-en-1-ol (515.89 mg, 7.15 mmol) and sodium bicarbonate (400.69 mg, 4.77 mmol) were added. The mixture was stirred at 130° C. for 4 h. After cooling the mixture to rt, the mixture was diluted with water and ethyl acetate. After filtration, the mixture was extracted with ethyl acetate and the combined organic layers were washed with water, brine, dried over anhydrous sodium sulfate, and concentrated. The residue was purified by silica gel chromatography, eluted with ethyl acetate/petroleum ether (0˜21% with 5% dichloromethane) to afford the title compound as an off-white solid.

### Step 2: tert-Butyl (1-((3-(3-(4-(3-(2,4-dioxotetrahydropyrimidin-1(2H)-yl)-1-methyl-1H-indazol-6-yl)piperidin-1-yl)-2-methylpropyl)phenyl)sulfonyl)piperidin-4-yl)carbamate

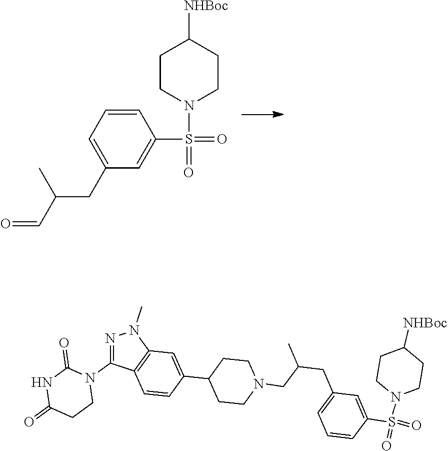 [SMILES: CC(C=O)Cc1cccc(S(=O)(=O)N2CCC(NC(=O)OC(C)(C)C)CC2)c1 ; CC(Cc1cccc(S(=O)(=O)N2CCC(NC(=O)OC(C)(C)C)CC2)c1)CN1CCC(c2ccc3c(N4CCC(=O)NC4=O)nn(C)c3c2)CC1]

A mixture of tert-butyl (1-((3-(2-methyl-3-oxopropyl)phenyl)sulfonyl)piperidin-4-yl)-carbamate (100 mg, 0.24 mmol) and 1-(1-methyl-6-(piperidin-4-yl)-1H-indazol-3-yl)dihydropyrimidine-2,4(1H,3H)-dione hydrochloride (88.63 mg, 0.24 mmol) in anhydrous dichloromethane (2 mL) was stirred at 25° C. for 3 h. Sodium triacetoxyborohydride (154.88 mg, 0.73 mmol) was added and the mixture was stirred 16 h. The mixture was diluted with water and extracted with ethyl acetate. The combined organic layers were washed with water, brine, dried over anhydrous sodium sulfate, and concentrated. The residue was purified by silica gel chromatography, eluted with methanol/dichloromethane (0˜5%) to afford the title compound as a white solid.

### Step 3: 1-(6-(1-(3-(3-((4-Aminopiperidin-1-yl)sulfonyl)phenyl)-2-methylpropyl)piperidin-4-yl)-1-methyl-1H-indazol-3-yl)dihydropyrimidine-2,4(1H,3H)-dione hydrochloride

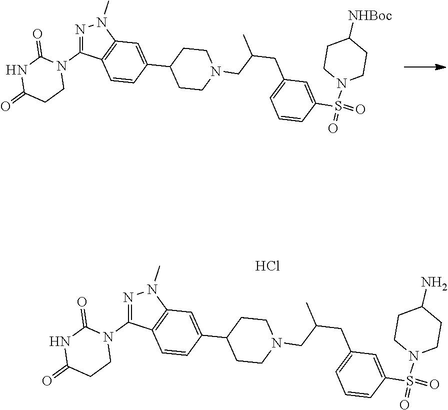 [SMILES: CC(Cc1cccc(S(=O)(=O)N2CCC(NC(=O)OC(C)(C)C)CC2)c1)CN1CCC(c2ccc3c(N4CCC(=O)NC4=O)nn(C)c3c2)CC1 ; CC(Cc1cccc(S(=O)(=O)N2CCC(N)CC2)c1)CN1CCC(c2ccc3c(N4CCC(=O)NC4=O)nn(C)c3c2)CC1 ; Cl]

To a stirred solution of tert-butyl (1-((3-(3-(4-(3-(2,4-dioxotetrahydropyrimidin-1(2H)-yl)-1-methyl-1H-indazol-6-yl)piperidin-1-yl)-2-methylpropyl)phenyl)sulfonyl)piperidin-4-yl)-carbamate (62 mg, 0.086 mmol) in dichloromethane (2 mL) was added 4 M hydrogen chloride in dioxane (1.0 mL) at 0° C. and the mixture was stirred for 1 h. The mixture was concentrated to afford the title compound as a white solid.

### Reference 62

### Synthesis of 1-(6-(1-(3-(3-((4-Aminopiperidin-1-yl)sulfonyl)phenyl)-2,2-dimethylpropyl) piperidin-4-yl)-1-methyl-1H-indazol-3-yl)dihydropyrimidine-2,4(1H,3H)-dione hydrochloride

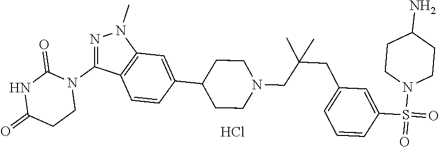 [SMILES: Cn1nc(N2CCC(=O)NC2=O)c2ccc(C3CCN(CC(C)(C)Cc4cccc(S(=O)(=O)N5CCC(N)CC5)c4)CC3)cc21 ; Cl]

### Step 1: tert-Butyl (1-((3-(2,2-dimethyl-3-oxopropyl)phenyl)sulfonyl)piperidin-4-yl)carbamate

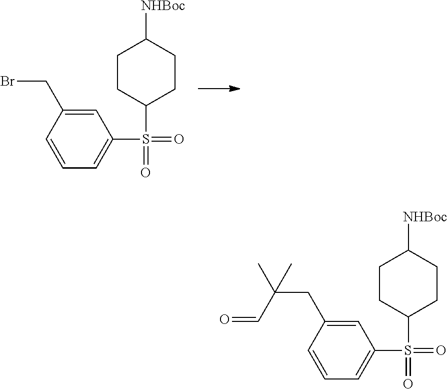 [SMILES: CC(C)(C)OC(=O)NC1CCC(S(=O)(=O)c2cccc(CBr)c2)CC1 ; CC(C)(C=O)Cc1cccc(S(=O)(=O)C2CCC(NC(=O)OC(C)(C)C)CC2)c1]

A mixture of tert-butyl N-[1-[3-(bromomethyl)phenyl]sulfonyl-4-piperidyl]carbamate (1.0 g, 2.31 mmol), 2-methylpropanal (416 mg, 5.77 mmol), tetrabutylammonium iodide (85.24 mg, 0.23 mmol) and sodium hydroxide (323.06 mg, 8.08 mmol) in 1,4-dioxane (10 mL) was heated to 70° C. and stirred for 3 h under Argon atmosphere. After cooling, the mixture was diluted with water and extracted with ethyl acetate. The combined organic layers were washed with water, brine, and dried over anhydrous sodium sulfate. After filtration, the filtrate was concentrated and the residue was purified by silica gel chromatography, eluted with ethyl acetate/petroleum ether (0˜20% with 5% dichloromethane) to afford the title compound as a white solid.

### Step 2: tert-Butyl (1-((3-(3-(4-(3-(2,4-dioxotetrahydropyrimidin-1(2H)-yl)-1-methyl-1H-indazol-6-yl)piperidin-1-yl)-2,2-dimethylpropyl)phenyl)sulfonyl)piperidin-4-yl)carbamate

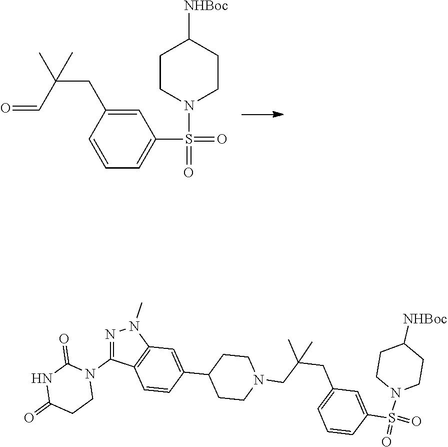 [SMILES: CC(C)(C=O)Cc1cccc(S(=O)(=O)N2CCC(NC(=O)OC(C)(C)C)CC2)c1 ; Cn1nc(N2CCC(=O)NC2=O)c2ccc(C3CCN(CC(C)(C)Cc4cccc(S(=O)(=O)N5CCC(NC(=O)OC(C)(C)C)CC5)c4)CC3)cc21]

Titanium tetraisopropanolate (1241.82 mg, 4.37 mmol) was added to a mixture of tert-butyl (1-((3-(2,2-dimethyl-3-oxopropyl)phenyl)sulfonyl)piperidin-4-yl)carbamate (530 mg, 1.25 mmol) and 1-(1-methyl-6-(piperidin-4-yl)-1H-indazol-3-yl)dihydropyrimidine-2,4(1H,3H)-dione hydrochloride (408.7 mg, 1.25 mmol) in anhydrous N-methyl-2-pyrrolidone (5.3 mL), and the mixture was heated to 90° C. for 3 h under Argon atmosphere. The mixture was cooled to rt and sodium cyanoborohydride (274.56 mg, 4.37 mmol) was added, and the mixture was stirred at 25° C. for 1 h. The mixture was diluted with water and extracted with ethyl acetate. The combined organic layers were washed with water, brine, dried over anhydrous sodium sulfate and concentrated. The residue was purified by silica gel chromatography, eluted with methanol/dichloromethane (0˜5%) to afford the title compound as a white solid.

### Step 3: 1-(6-(1-(3-(3-((4-Aminopiperidin-1-yl)sulfonyl)phenyl)-2,2-dimethylpropyl)piperidin-4-yl)-1-methyl-1H-indazol-3-yl)dihydropyrimidine-2,4(1H,3H)-dione hydrochloride

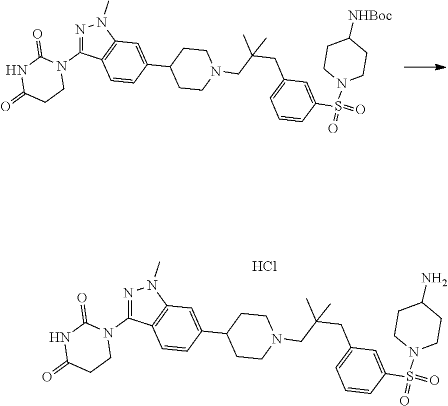 [SMILES: Cn1nc(N2CCC(=O)NC2=O)c2ccc(C3CCN(CC(C)(C)Cc4cccc(S(=O)(=O)N5CCC(NC(=O)OC(C)(C)C)CC5)c4)CC3)cc21 ; Cn1nc(N2CCC(=O)NC2=O)c2ccc(C3CCN(CC(C)(C)Cc4cccc(S(=O)(=O)N5CCC(N)CC5)c4)CC3)cc21 ; Cl]

To a stirred solution of tert-butyl (1-((3-(3-(4-(3-(2,4-dioxotetrahydropyrimidin-1(2H)-yl)-1-methyl-1H-indazol-6-yl)piperidin-1-yl)-2,2-dimethylpropyl)phenyl)sulfonyl)piperidin-4-yl)carbamate (280 mg, 0.38 mmol) in dichloromethane (3 mL) was added 4 M hydrogen chloride in dioxane (1.5 mL) at 0° C. and stirred for 1 h. The solvent was removed under reduced pressure to afford title compound as a white solid.

### Example 1

### Synthesis of 1-(6-(1-(3-((4-((5-(difluoromethyl)pyrimidin-2-yl)amino)piperidin-1-yl)sulfonyl)-benzyl)piperidin-4-yl)-1-methyl-1H-indazol-3-yl)dihydropyrimidine-2,4(1H,3H)-dione

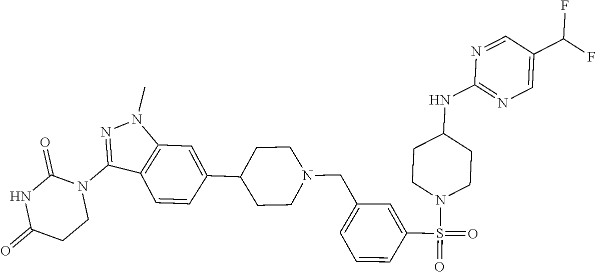 [SMILES: Cn1nc(N2CCC(=O)NC2=O)c2ccc(C3CCN(Cc4cccc(S(=O)(=O)N5CCC(Nc6ncc(C(F)F)cn6)CC5)c4)CC3)cc21]

A mixture of 1-(6-(1-(3-((4-aminopiperidin-1-yl)sulfonyl)benzyl)piperidin-4-yl)-1-methyl-1H-indazol-3-yl)dihydropyrimidine-2,4(1H,3H)-dione (45 mg, 0.08 mmol, 1.00 eq.), DIEA (20 mg, 0.16 mmol, 2.00 eq.) and 2-chloro-5-(difluoromethyl)pyrimidine (19 mg, 0.09 mmol, 1.20 eq.) in DMSO (2.0 mL) was stirred at 90° C. for 12 h. The mixture was diluted with water and extracted with EtOAc. The combined organic layer was washed with brine, dried over anhydrous Na₂SO₄ and concentrated. The residue was purified by prep-TLC to give the title compound as a white solid. MS (ES, m/z): [M+1]⁺=708.1.

The following compounds were synthesized by proceeding analogously as described in Example 1.

| Ex# | Structure | Synthesis |
|---|---|---|
| 2 | 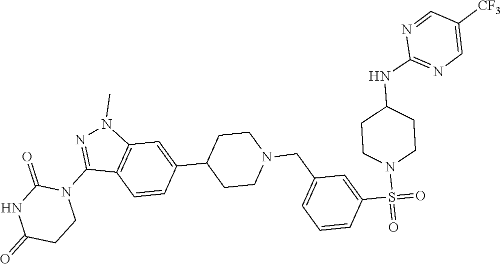 [SMILES: Cn1nc(N2CCC(=O)NC2=O)c2ccc(C3CCN(Cc4cccc(S(=O)(=O)N5CCC(Nc6ncc(C(F)(F)F)cn6)CC5)c4)CC3)cc21] | 2-chloro-5-(trifluoromethyl)- pyrimidine replaced 2-chloro- 5-(difluoromethyl)pyrimidine |
| 3 | 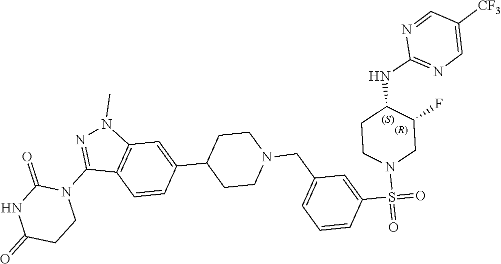 | Ref. 54a replaced Ref. 54, and 2-chloro-5-(trifluoromethyl)- pyrimidine replaced 2-chloro- 5-(difluoromethyl)pyrimidine |
| 4 | 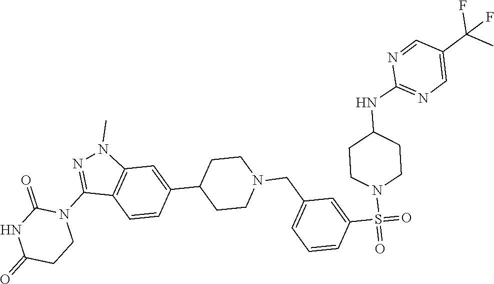 [SMILES: Cn1nc(N2CCC(=O)NC2=O)c2ccc(C3CCN(Cc4cccc(S(=O)(=O)N5CCC(Nc6ncc(C(C)(F)F)cn6)CC5)c4)CC3)cc21] | Ref. 48a replaced 2-chloro-5- (difluoromethyl)-pyrimidine |
| 5 | 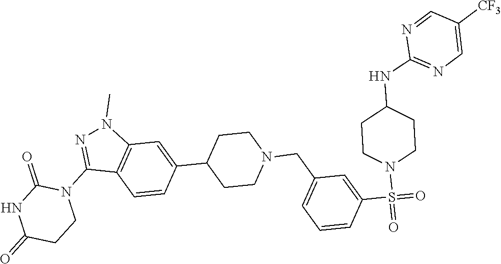 [SMILES: Cn1nc(N2CCC(=O)NC2=O)c2ccc(C3CCN(Cc4cccc(S(=O)(=O)N5CCC(Nc6ncc(C(F)(F)F)cn6)CC5)c4)CC3)cc21] | 2-chloropyrimidine-5- carbonitrile replaced 2-chloro- 5-(difluoromethyl)pyrimidine |
| 6 | 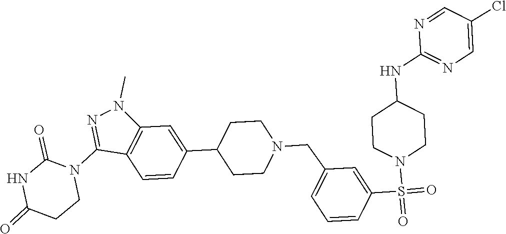 [SMILES: Cn1nc(N2CCC(=O)NC2=O)c2ccc(C3CCN(Cc4cccc(S(=O)(=O)N5CCC(Nc6ncc(Cl)cn6)CC5)c4)CC3)cc21] | 2,5-dichloropyrimidine replaced 2-chloro-5- (difluoromethyl)pyrimidine |
| 7 | 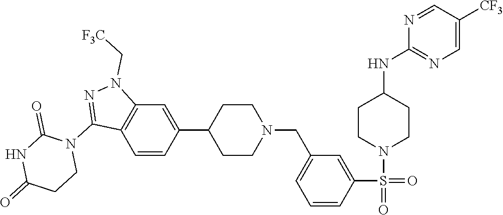 [SMILES: O=C1CCN(c2nn(CC(F)(F)F)c3cc(C4CCN(Cc5cccc(S(=O)(=O)N6CCC(Nc7ncc(C(F)(F)F)cn7)CC6)c5)CC4)ccc23)C(=O)N1] | 2-chloro-5-(trifluoromethyl)- pyrimidinereplaced 2-chloro- 5-(difluoromethyl)pyrimidine and Ref 54c replaced Ref. 54 |
| 8 | 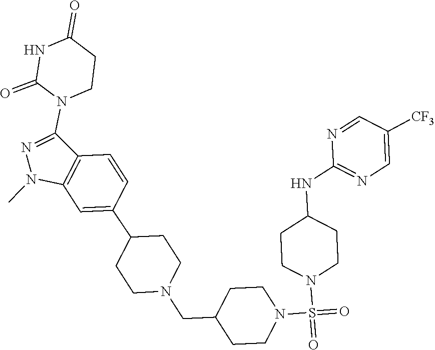 [SMILES: Cn1nc(N2CCC(=O)NC2=O)c2ccc(C3CCN(CC4CCN(S(=O)(=O)N5CCC(Nc6ncc(C(F)(F)F)cn6)CC5)CC4)CC3)cc21] | 2-chloro-5-(trifluoromethyl)- pyrimidinereplaced 2-chloro- 5-(difluoromethyl)pyrimidine and Ref 55 replaced Ref. 54 |
| 9 | 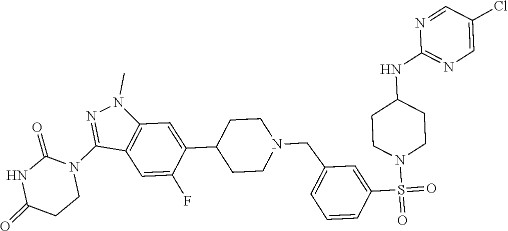 [SMILES: Cn1nc(N2CCC(=O)NC2=O)c2cc(F)c(C3CCN(Cc4cccc(S(=O)(=O)N5CCC(Nc6ncc(Cl)cn6)CC5)c4)CC3)cc21] | 2,5-dichloropyrimidine replaced 2-chloro-5-(difluoro- methyl)pyrimidine and Ref 54b replaced Ref. 54 |
| 10 | 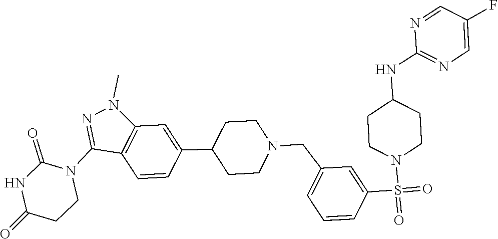 [SMILES: Cn1nc(N2CCC(=O)NC2=O)c2ccc(C3CCN(Cc4cccc(S(=O)(=O)N5CCC(Nc6ncc(F)cn6)CC5)c4)CC3)cc21] | 2-chloro-5-fluoropyrimidine replaced 2-chloro-5-(difluoro- methyl)pyrimidine |
| 11 | 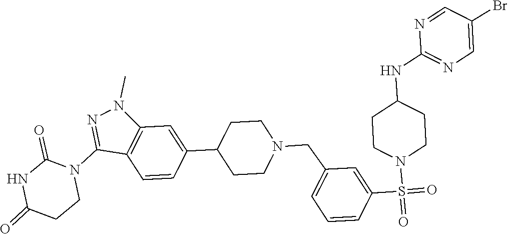 [SMILES: Cn1nc(N2CCC(=O)NC2=O)c2ccc(C3CCN(Cc4cccc(S(=O)(=O)N5CCC(Nc6ncc(Br)cn6)CC5)c4)CC3)cc21] | 2-chloro-5-bromopyrimidine replaced 2-chloro-5-(difluoro- methyl)pyrimidine |
| 12 | 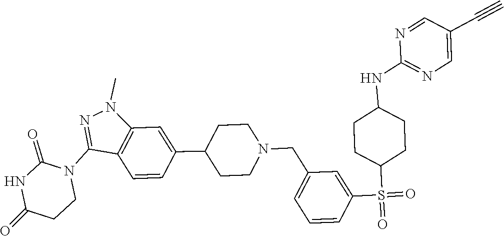 [SMILES: C#Cc1cnc(NC2CCC(S(=O)(=O)c3cccc(CN4CCC(c5ccc6c(N7CCC(=O)NC7=O)nn(C)c6c5)CC4)c3)CC2)nc1] | 2-chloro-5-ethynylpyrimidine replaced 2-chloro-5-(difluoro- methyl)pyrimidine |
| 13 | 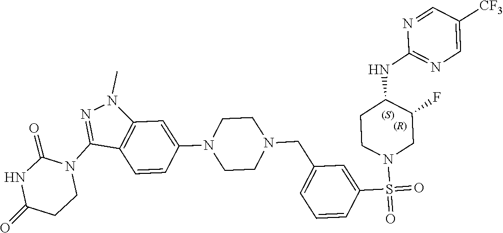 | 2,5-dichloropyrimidine replaced 2-chloro-5-(difluoro- methyl)pyrimidine and Ref 54a replaced Ref. 54 |
| 14 | 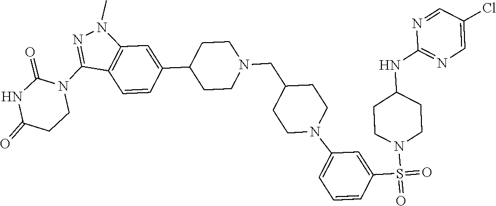 [SMILES: Cn1nc(N2CCC(=O)NC2=O)c2ccc(C3CCN(CC4CCN(c5cccc(S(=O)(=O)N6CCC(Nc7ncc(Cl)cn7)CC6)c5)CC4)CC3)cc21] | 2,5-dichloropyrimidine replaced 2-chloro-5-(difluoro- methyl)pyrimidine and Ref 56 replaced Ref. 54 |
| 15 | 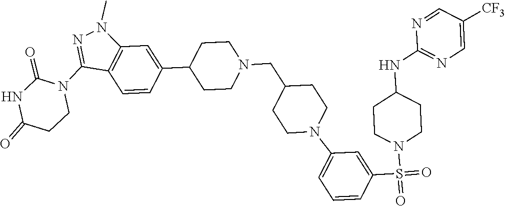 [SMILES: Cn1nc(N2CCC(=O)NC2=O)c2ccc(C3CCN(CC4CCN(c5cccc(S(=O)(=O)N6CCC(Nc7ncc(C(F)(F)F)cn7)CC6)c5)CC4)CC3)cc21] | 2-chloro-5-(trifluoromethyl)- pyrimidine replaced 2-chloro- 5-(difluoromethyl)pyrimidine and Ref 56 replaced Ref. 54 |
| 16 | 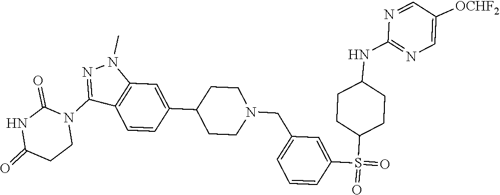 [SMILES: Cn1nc(N2CCC(=O)NC2=O)c2ccc(C3CCN(Cc4cccc(S(=O)(=O)C5CCC(Nc6ncc(OC(F)F)cn6)CC5)c4)CC3)cc21] | Ref 49. replaced 2-chloro-5- (difluoromethyl)-pyrimidine |
| 17 | 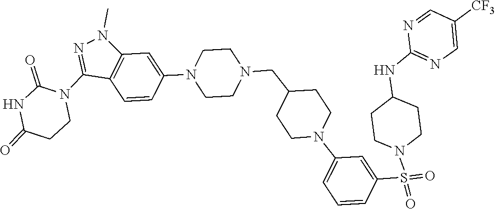 [SMILES: Cn1nc(N2CCC(=O)NC2=O)c2ccc(N3CCN(CC4CCN(c5cccc(S(=O)(=O)N6CCC(Nc7ncc(C(F)(F)F)cn7)CC6)c5)CC4)CC3)cc21] | 2-chloro-5-(trifluoromethyl)- pyrimidinereplaced 2-chloro- 5-(difluoromethyl)pyrimidine and Ref 56a replaced Ref. 56 |
| 18 | 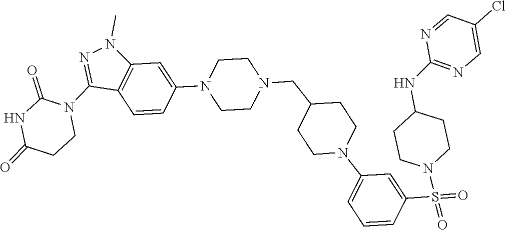 [SMILES: Cn1nc(N2CCC(=O)NC2=O)c2ccc(N3CCN(CC4CCN(c5cccc(S(=O)(=O)N6CCC(Nc7ncc(Cl)cn7)CC6)c5)CC4)CC3)cc21] | 2,5-dichloropyrimidine replaced 2-chloro-5-(difluoro- methyl)pyrimidine and Ref 56a replaced Ref. 56 |

### Example 19

### Synthesis of 1-(1-methyl-6-(1-(3-((4-((5-methylpyrimidin-2-yl)amino)piperidin-1-yl)sulfonyl)-benzyl)piperidin-4-yl)-1H-indazol-3-yl)dihydropyrimidine-2,4(1H,3H)-dione

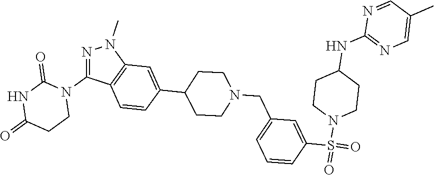 [SMILES: Cc1cnc(NC2CCN(S(=O)(=O)c3cccc(CN4CCC(c5ccc6c(N7CCC(=O)NC7=O)nn(C)c6c5)CC4)c3)CC2)nc1]

### Step 1: tert-Butyl 4-((5-methylpyrimidin-2-yl)amino)piperidine-1-carboxylate

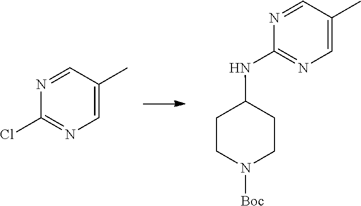

A mixture of 2-chloro-5-methylpyrimidine (300 mg, 2.35 mmol, 1.00 eq), DIEA (910 mg, 7.05 mmol, 3.00 eq.), tert-butyl 4-aminopiperidine-1-carboxylate (721 mg, 3.60 mmol, 1.50 eq.) in DMSO (3.0 mL) was stirred at 90° C. for 12 h. The mixture was quenched with H₂O and extracted with EA. The combined organic layer was concentrated and the residue was purified by silica gel column chromatography, eluted with PE/EA (3:1), to afford the title compound as a white solid.

### Step 2: 5-Methyl-N-(piperidin-4-yl)pyrimidin-2-amine trifluoroacetate

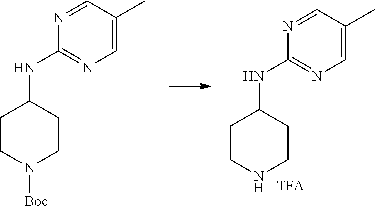 [SMILES: Cc1cnc(NC2CCN(C(=O)OC(C)(C)C)CC2)nc1 ; Cc1cnc(NC2CCNCC2)nc1]

To a solution of tert-butyl 4-((5-methylpyrimidin-2-yl)amino)piperidine-1-carboxylate (420 mg, 1.44 mmol, 1.00 eq.) in DCM (2.0 mL) was added TFA (0.5 mL) and the mixture was stirred at r.t for 2 h. The mixture was concentrated to give the title compound as a yellow oil.

### Step 3: N-(1-((3-(Bromomethyl)phenyl)sulfonyl)piperidin-4-yl)-5-methylpyrimidin-2-amine

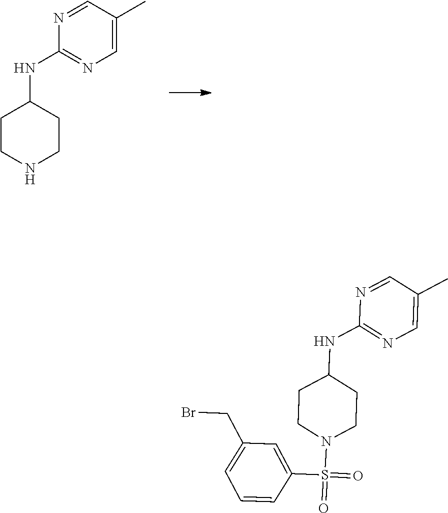 [SMILES: Cc1cnc(NC2CCNCC2)nc1 ; Cc1cnc(NC2CCN(S(=O)(=O)c3cccc(CBr)c3)CC2)nc1]

3-(Bromomethyl)benzene-1-sulfonyl chloride (232 mg, 0.86 mmol, 1.20 eq.) in THF (1.0 mL) was added to a stirred solution of 5-methyl-N-(piperidin-4-yl)pyrimidin-2-amine trifluoroacetate (138 mg, 0.72 mmol, 1.00 eq.), TEA (436 mg, 4.32 mmol, 6.00 eq.) in THF (3.0 mL) at 0° C. and the mixture was stirred at 0° C. for 1 h. The mixture was quenched with H₂O and extracted with DCM. The combined organic layer was concentrated and the residue was purified by silica gel column chromatography, eluted with PE/EA (1:1), to afford the title compound as a yellow solid.

### Step 4: 1-(1-Methyl-6-(1-(3-((4-((5-methylpyrimidin-2-yl)amino)piperidin-1-yl)sulfonyl)-benzyl)piperidin-4-yl)-1H-indazol-3-yl)dihydropyrimidine-2,4(1H,3H)-dione

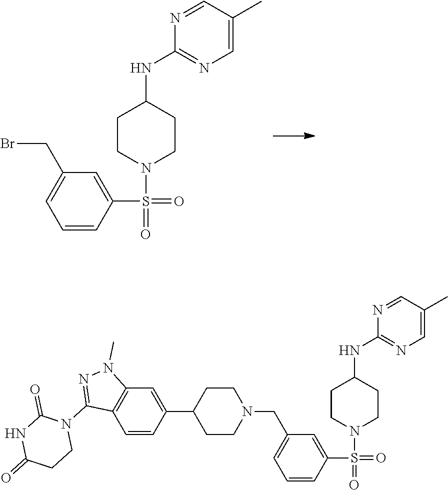 [SMILES: Cc1cnc(NC2CCN(S(=O)(=O)c3cccc(CBr)c3)CC2)nc1 ; Cc1cnc(NC2CCN(S(=O)(=O)c3cccc(CN4CCC(c5ccc6c(N7CCC(=O)NC7=O)nn(C)c6c5)CC4)c3)CC2)nc1]

N-(1-((3-(bromomethyl)phenyl)sulfonyl)piperidin-4-yl)-5-methylpyrimidin-2-amine (119 mg, 0.28 mmol, 1.20 eq.) was added to a stirred solution of N-(1-((3-(bromomethyl)phenyl)-sulfonyl)piperidin-4-yl)-5-methylpyrimidin-2-amine (77 mg, 0.234 mmol, 1.00 eq.) and TEA (71 mg, 0.70 mmol, 3.00 eq.) in THF (2.0 mL), and the mixture was stirred at rt for 12 h. The mixture was filtered and the filtrate was concentrated and the residue was purified by prep-TLC on silica gel (DCM:MeOH=20:1) to give the title compound as white solid. MS (ES, m/z): [M+1]⁺=672.1.

The following compounds were synthesized by proceeding analogously as described in Example 19.

| Ex# | Structure | Synthesis |
|---|---|---|
| 20 | 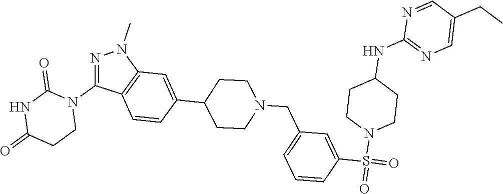 [SMILES: CCc1cnc(NC2CCN(S(=O)(=O)c3cccc(CN4CCC(c5ccc6c(N7CCC(=O)NC7=O)nn(C)c6c5)CC4)c3)CC2)nc1] | 2-chloro-5-ethylpyrimidine replaced 2-chloro-5-methyl- pyrimidine in Step 1 |

### Example 21

### Synthesis of 1-(5-fluoro-1-methyl-6-(1-(3-((4-((5-(trifluoromethyl)pyrimidin-2-yl)amino)-piperidin-1-yl)sulfonyl)benzyl)piperidin-4-yl)-1H-indazol-3-yl)dihydropyrimidine-2,4(1H,3H)-dione

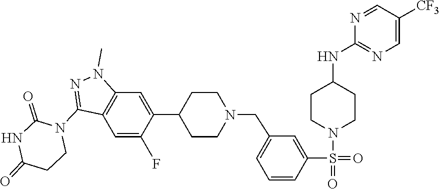 [SMILES: Cn1nc(N2CCC(=O)NC2=O)c2cc(F)c(C3CCN(Cc4cccc(S(=O)(=O)N5CCC(Nc6ncc(C(F)(F)F)cn6)CC5)c4)CC3)cc21]

### Step 1: tert-Butyl 4-((5-(trifluoromethyl)pyrimidin-2-yl)amino)piperidine-1-carboxylate

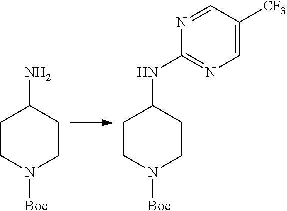 [SMILES: CC(C)(C)OC(=O)N1CCC(N)CC1 ; CC(C)(C)OC(=O)N1CCC(Nc2ncc(C(F)(F)F)cn2)CC1]

2-Chloro-5-(trifluoromethyl)pyrimidine (3.30 g, 16.40 mmol, 1.00 eq.) was added to a solution of tert-butyl 4-aminopiperidine-1-carboxylate (3.00 g, 16.40 mmol, 1.00 eq.) and DIEA (6.40 g, 49.20 mmol, 3.00 eq.) in DMSO (30.0 mL), and the mixture was stirred at 90° C. for 12 h. The mixture was quenched with H₂O and extracted with EA. The organic layer was concentrated and purified by silica gel column chromatography, eluted with PE/EA (3:1), to afford the title compound as a white solid.

### Step 2: N-(Piperidin-4-yl)-5-(trifluoromethyl)pyrimidin-2-amine trifluoroacetate

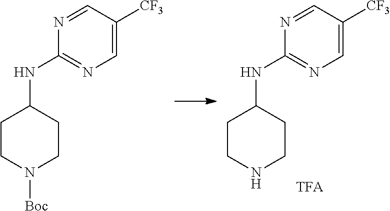 [SMILES: CC(C)(C)OC(=O)N1CCC(Nc2ncc(C(F)(F)F)cn2)CC1 ; FC(F)(F)c1cnc(NC2CCNCC2)nc1]

TFA (0.5 mL) was added to a solution of tert-butyl 4-((5-(trifluoromethyl)pyrimidin-2-yl)amino)piperidine-1-carboxylate (1.00 g, 2.89 mmol, 1.00 eq.) in DCM (2.0 mL) dropwise and the solution was stirred at r.t for 2 h. The mixture was concentrated to give the title compound as a yellow oil.

### Step 3: N-(1-((3-(Bromomethyl)phenyl)sulfonyl)piperidin-4-yl)-5-(trifluoromethyl)pyrimidin-2-amine

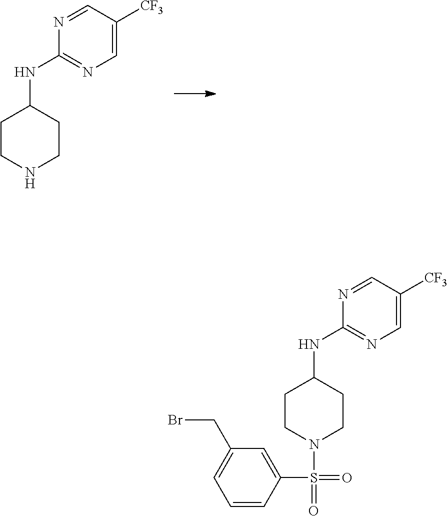 [SMILES: FC(F)(F)c1cnc(NC2CCNCC2)nc1 ; O=S(=O)(c1cccc(CBr)c1)N1CCC(Nc2ncc(C(F)(F)F)cn2)CC1]

The title compound was synthesized by proceeding analogously as described in Example 19, Step 3.

### Step 4: 1-(5-Fluoro-1-methyl-6-(1-(3-((4-((5-(trifluoromethyl)pyrimidin-2-yl)amino)piperidin-1-yl)sulfonyl)benzyl)piperidin-4-yl)-1H-indazol-3-yl)dihydropyrimidine-2,4(1H,3H)-dione

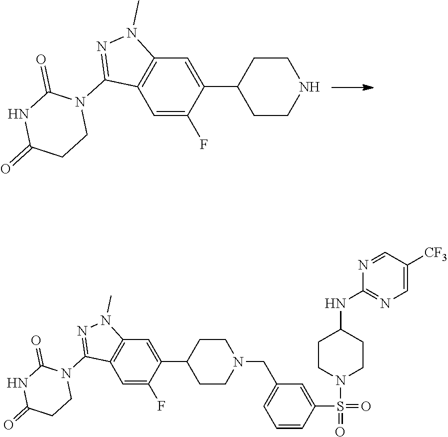 [SMILES: Cn1nc(N2CCC(=O)NC2=O)c2cc(F)c(C3CCNCC3)cc21 ; Cn1nc(N2CCC(=O)NC2=O)c2cc(F)c(C3CCN(Cc4cccc(S(=O)(=O)N5CCC(Nc6ncc(C(F)(F)F)cn6)CC5)c4)CC3)cc21]

N-(1-((3-(Bromomethyl)phenyl)sulfonyl)piperidin-4-yl)-5-(trifluoromethyl)pyrimidin-2-amine (200 mg, 0.446 mmol, 2.00 eq.) was added to a mixture of 1-(5-fluoro-1-methyl-6-(piperidin-4-yl)-1H-indazol-3-yl)dihydropyrimidine-2,4(1H,3H)-dione (77 mg, 0.223 mmol, 1.00 eq.), and TEA (68 mg, 0.669 mmol, 3.00 eq.) in THF (3.0 mL), and the mixture was stirred at rt for 12 h. The mixture was quenched with H₂O and extracted with DCM. The combined organic layer was concentrated and the residue was purified by prep-TLC to afford the title compound as a white solid. MS (ES, m/z): [M+1]⁺=744.1.

The following compounds were synthesized by proceeding analogously as described in Example 21.

| Ex# | Structure | Synthesis |
|---|---|---|
| 22 | 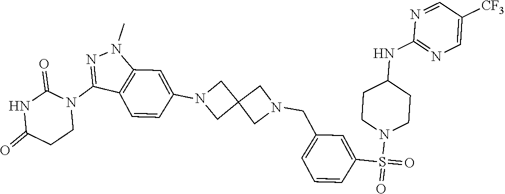 [SMILES: Cn1nc(N2CCC(=O)NC2=O)c2ccc(N3CC4(CN(Cc5cccc(S(=O)(=O)N6CCC(Nc7ncc(C(F)(F)F)cn7)CC6)c5)C4)C3)cc21] | Ref. 57 replaced Ref. 45b in Step 4 |
| 23 | 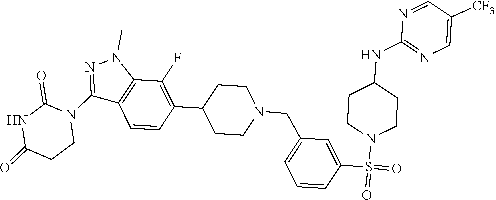 [SMILES: Cn1nc(N2CCC(=O)NC2=O)c2ccc(C3CCN(Cc4cccc(S(=O)(=O)N5CCC(Nc6ncc(C(F)(F)F)cn6)CC5)c4)CC3)c(F)c21] | Ref. 45a replaced Ref. 45b in Step 4 |
| 24 | 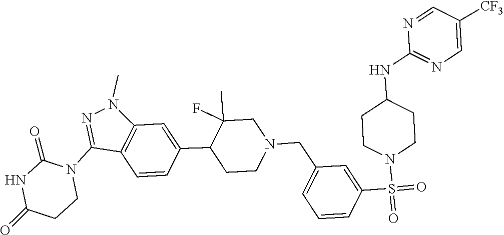 [SMILES: Cn1nc(N2CCC(=O)NC2=O)c2ccc(C3CCN(Cc4cccc(S(=O)(=O)N5CCC(Nc6ncc(C(F)(F)F)cn6)CC5)c4)CC3(C)F)cc21] | Ref. 53 replaced Ref. 45b in Step 4 |
| 25 | 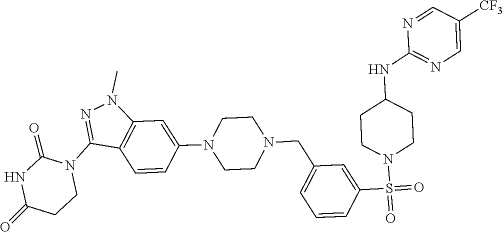 [SMILES: Cn1nc(N2CCC(=O)NC2=O)c2ccc(N3CCN(Cc4cccc(S(=O)(=O)N5CCC(Nc6ncc(C(F)(F)F)cn6)CC5)c4)CC3)cc21] | Ref. 51 replaced Ref. 45b in Step 4 |
| 26 | 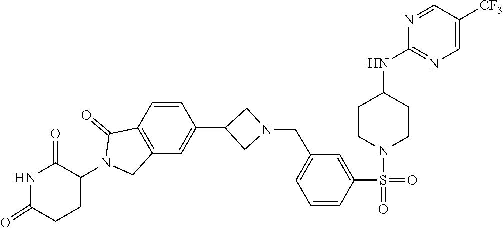 [SMILES: O=C1CCC(N2Cc3cc(C4CN(Cc5cccc(S(=O)(=O)N6CCC(Nc7ncc(C(F)(F)F)cn7)CC6)c5)C4)ccc3C2=O)C(=O)N1] | Ref. 31 replaced Ref. 45b in Step 4 |

### Example 27

### Synthesis of 1-(6-(1-(1-(3-((4-((5-chloropyrimidin-2-yl)amino)piperidin-1-yl)sulfonyl)-phenyl)ethyl)piperidin-4-yl)-1-methyl-1H-indazol-3-yl)dihydropyrimidine-2,4(1H,3H)-dione

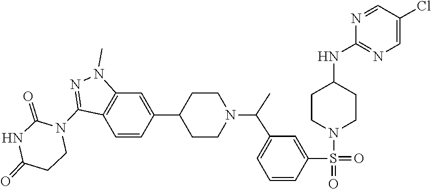 [SMILES: CC(c1cccc(S(=O)(=O)N2CCC(Nc3ncc(Cl)cn3)CC2)c1)N1CCC(c2ccc3c(N4CCC(=O)NC4=O)nn(C)c3c2)CC1]

### Step 1:(4-(tert-Butyl)benzyl)(3-ethylphenyl)sulfane

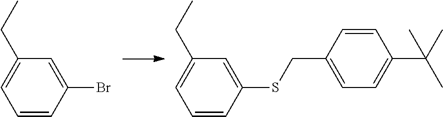

A mixture of 1-bromo-3-ethylbenzene (2.50 g, 13.51 mmol, 1.00 eq.), (4-(tert-butyl)phenyl) methanethiol (3.45 g, 20.27 mmol, 1.50 eq.), DIEA (5.22 g, 40.5 mmol, 3.00 eq.), Xantphos (1.56 g, 2.7 mmol, 0.20 eq.) and Pd₂(dba)₃ (1.22 g, 1.35 mmol, 0.10 eq.) in dioxane (20.0 mL) was stirred at 100° C. under N₂ for 16 h. The mixture was diluted with water and extracted EA. The organic layer was washed with brine, dried over Na₂SO₄, concentrated and the residue was purified by column chromatography on silica gel (PE:EA=5:1) to give the title compound as a colorless oil.

### Step 2: 3-Ethylbenzenesulfonyl chloride

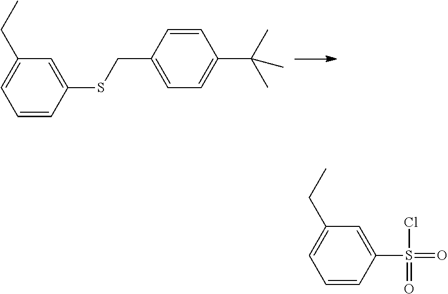 [SMILES: CCc1cccc(SCc2ccc(C(C)(C)C)cc2)c1 ; CCc1cccc(S(=O)(=O)Cl)c1]

1,3-Dichloro-5,5-dimethylimidazolidine-2,4-dione (1.39 g, 7.04 mmol, 2.00 eq.) was added to a solution of (4-(tert-butyl)benzyl)(3-ethylphenyl)sulfane (1.00 g, 3.52 mmol, 1.00 eq.) in MeCN/H₂O/AcOH=7:3:1 (33.0 mL) and the mixture was stirred at 80° C. for 16 h. The mixture was diluted with water and extracted EA. The organic layer was washed with brine, dried over Na₂SO₄, concentrated and the residue was purified by column chromatography on silica gel (PE:EA=10:1) to give the title compound as a colorless oil.

### Step 3: 3-(1-Bromoethyl)benzenesulfonyl chloride

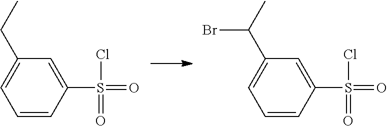

A mixture of 3-ethylbenzenesulfonyl chloride (294 mg, 2.94 mmol, 1.00 eq.), NBS (532 mg, 2.94 mmol, 1.00 eq.), and AIBN (50 mg, 0.30 mmol, 0.10 eq.) in ACN (10.0 ml) was stirred at 80° C. under N₂ for 16 h. The mixture was diluted with water and extracted with EA. The organic layer was washed with brine, dried over Na₂SO₄, concentrated and purified by column chromatography on silica gel (PE:EA=10:1) to give the title compound as a yellow oil.

### Step 4: N-(1-((3-(1-Bromoethyl)phenyl)sulfonyl)piperidin-4-yl)-5-chloropyrimidin-2-amine

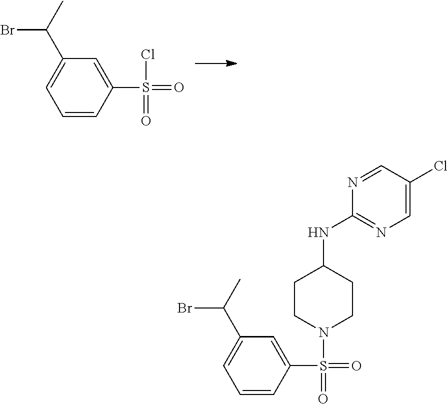 [SMILES: CC(Br)c1cccc(S(=O)(=O)Cl)c1 ; CC(Br)c1cccc(S(=O)(=O)N2CCC(Nc3ncc(Cl)cn3)CC2)c1]

3-(1-Bromoethyl)benzenesulfonyl chloride (295 mg, 1.04 mmol, 1.00 eq.) in DCM (3.0 mL) was dropped slowly to a mixture of 5-chloro-N-(piperidin-4-yl)pyrimidin-2-amine (220 mg, 1.04 mmol, 1.00 eq.) in DCM (2.0 mL) and TEA (320 mg, 3.14 mmol, 3.00 eq.) at 0° C. and the mixture was stirred for 3 h. The mixture was diluted with water and extracted with DCM. The combined organic layer was washed with brine, dried over Na₂SO₄ and concentrated. The residue was purified by silica gel column chromatography eluted with PE/EtOAc (2:1) to afford the title compound as a white solid.

### Step 5: 1-(6-(1-(1-(3-((4-((5-Chloropyrimidin-2-yl)amino)piperidin-1-yl)sulfonyl)phenyl)ethyl)-piperidin-4-yl)-1-methyl-1H-indazol-3-yl)dihydropyrimidine-2,4(1H,3H)-dione

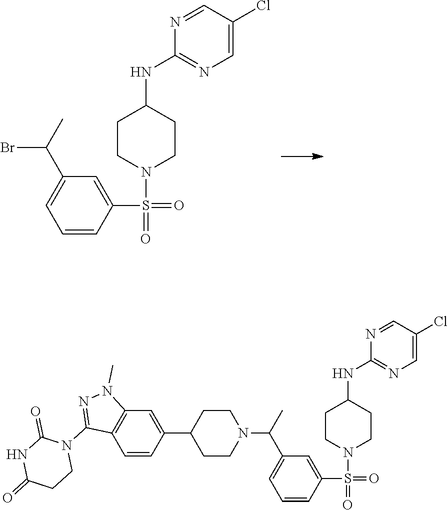 [SMILES: CC(Br)c1cccc(S(=O)(=O)N2CCC(Nc3ncc(Cl)cn3)CC2)c1 ; CC(c1cccc(S(=O)(=O)N2CCC(Nc3ncc(Cl)cn3)CC2)c1)N1CCC(c2ccc3c(N4CCC(=O)NC4=O)nn(C)c3c2)CC1]

A mixture of 1-(1-methyl-6-(piperidin-4-yl)-1H-indazol-3-yl)dihydropyrimidine-2,4(1H,3H)-dione (76 mg, 0.23 mmol, 1.00 eq.), TEA (71 mg, 0.70 mmol, 3.00 eq.), and N-(1-((3-(1-bromoethyl)phenyl)sulfonyl)piperidin-4-yl)-5-chloropyrimidin-2-amine (108 mg, 0.23 mmol, 1.00 eq.) in THF/DMF (2.0 mL/0.5 mL) was stirred at 55° C. under nitrogen atmosphere for 20 h. The mixture was diluted with water and extracted EA. The combined organic layer was washed with brine, dried over anhydrous Na₂SO₄, filtered, and then concentrated. The residue was purified by silica gel column chromatography, eluted with DCM/MeOH (20:1), to afford the title compound as a white solid. MS (ES, m/z): [M+1]⁺=706.1.

### Example 28

### Synthesis of 1-(1-methyl-6-(1-(3-(1-((4-((5-(trifluoromethyl)pyrimidin-2-yl)amino)piperidin-1-yl)sulfonyl)piperidin-4-yl)propyl)piperidin-4-yl)-1H-indazol-3-yl)dihydropyrimidine-2,4(1H,3H)-dione

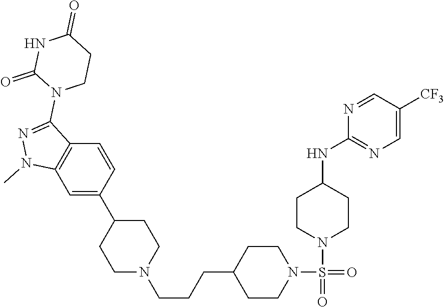 [SMILES: Cn1nc(N2CCC(=O)NC2=O)c2ccc(C3CCN(CCCC4CCN(S(=O)(=O)N5CCC(Nc6ncc(C(F)(F)F)cn6)CC5)CC4)CC3)cc21]

### Step 1: tert-Butyl 4-(3-(4-(3-(2,4-dioxotetrahydropyrimidin-1(2H)-yl)-1-methyl-1H-indazol-6-yl)piperidin-1-yl)propyl)piperidine-1-carboxylate

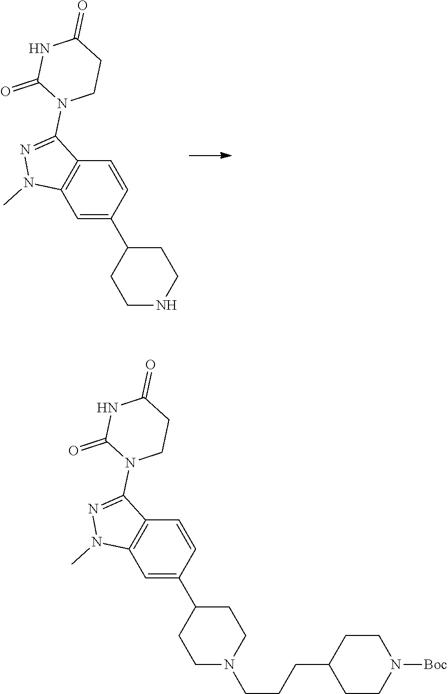 [SMILES: Cn1nc(N2CCC(=O)NC2=O)c2ccc(C3CCNCC3)cc21 ; Cn1nc(N2CCC(=O)NC2=O)c2ccc(C3CCN(CCCC4CCN(C(=O)OC(C)(C)C)CC4)CC3)cc21]

To a solution of 1-(1-methyl-6-(piperidin-4-yl)-1H-indazol-3-yl)dihydropyrimidine-2,4(1H,3H)-dione (140 mg, 0.43 mmol, 1.00 eq.) in DMF (2.5 mL) was added AcOH (3 drops) and tert-butyl 4-(3-oxopropyl)piperidine-1-carboxylate (125 mg, 0.52 mmol, 1.20 eq.), and the mixture was stirred at 45° C. for 45 min. The mixture was cooled to r.t, NaBH₃CN (54 mg, 0.86 mmol, 2.00 eq.) was added and the mixture was stirred at r.t overnight. The mixture was diluted with water and extracted with EtOAc. The combined organic layer was washed with brine, dried over anhydrous Na₂SO₄, and concentrated. The residue was purified by silica gel chromatography, eluted with DCM/MeOH (20:1), to afford the title compound as a white solid.

### Step 2: 4-((5-(Trifluoromethyl)pyrimidin-2-yl)amino)piperidine-1-sulfonyl chloride

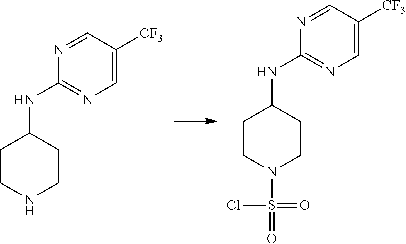 [SMILES: FC(F)(F)c1cnc(NC2CCNCC2)nc1 ; O=S(=O)(Cl)N1CCC(Nc2ncc(C(F)(F)F)cn2)CC1]

Sulfuryl dichloride (177 mg, 1.31 mmol, 1.50 eq.) in DCM (2.0 mL) was added to a stirred solution of N-(piperidin-4-yl)-5-(trifluoromethyl)pyrimidin-2-amine (214 mg, 0.87 mmol, 1.00 eq.) and DIEA (449 mg, 3.48 mmol, 4.00 eq.) in DCM (2.0 mL) at −78° C. The mixture was allowed to warm to room temperature and stirred for 12 h, and the mixture was quenched with H₂O and then extracted with DCM. The combined organic layer was washed with water, dried over anhydrous Na₂SO₄, filtered, and then concentrated. The residue was purified by silica gel column chromatography, eluted with PE/EA (3:1), to afford the title compound as white solid.

### Step 3: 1-(1-Methyl-6-(1-(3-(piperidin-4-yl)propyl)piperidin-4-yl)-1H-indazol-3-yl)dihydropyrimidine-2,4(1H,3H)-dione trifluoroacetate

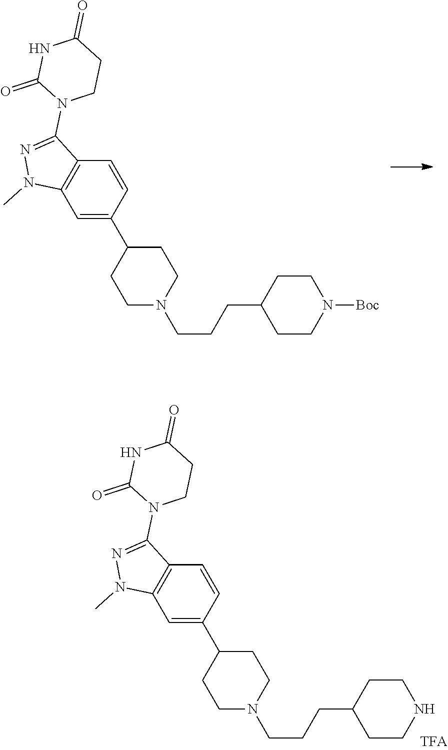 [SMILES: Cn1nc(N2CCC(=O)NC2=O)c2ccc(C3CCN(CCCC4CCN(C(=O)OC(C)(C)C)CC4)CC3)cc21 ; Cn1nc(N2CCC(=O)NC2=O)c2ccc(C3CCN(CCCC4CCNCC4)CC3)cc21]

A mixture of tert-butyl 4-(3-(4-(3-(2,4-dioxotetrahydropyrimidin-1(2H)-yl)-1-methyl-1H-indazol-6-yl)piperidin-1-yl)propyl)piperidine-1-carboxylate (20 mg, 0.04 mmol, 1.00 eq.) in TFA/DCM (0.5 mL/2 mL) was stirred at rt for 2 h. The mixture was concentrated to give the title compound as brown oil.

### Step 4: 1-(1-Methyl-6-(1-(3-(1-((4-((5-(trifluoromethyl)pyrimidin-2-yl)amino)piperidin-1-yl)sulfonyl)piperidin-4-yl)propyl)piperidin-4-yl)-1H-indazol-3-yl)dihydropyrimidine-2,4(1H,3H)-dione

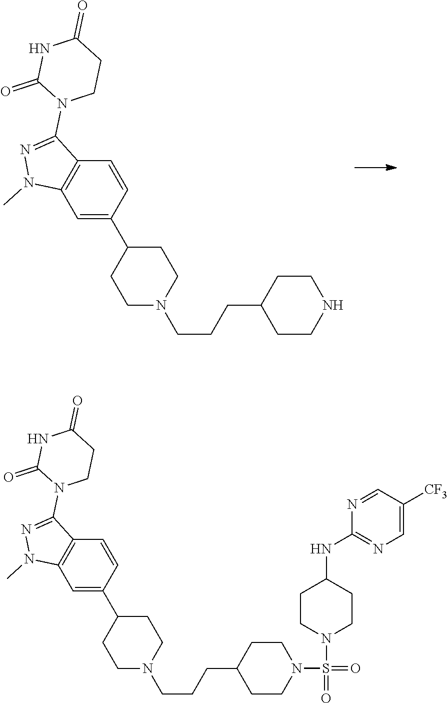 [SMILES: Cn1nc(N2CCC(=O)NC2=O)c2ccc(C3CCN(CCCC4CCNCC4)CC3)cc21 ; Cn1nc(N2CCC(=O)NC2=O)c2ccc(C3CCN(CCCC4CCN(S(=O)(=O)N5CCC(Nc6ncc(C(F)(F)F)cn6)CC5)CC4)CC3)cc21]

4-((5-(Trifluoromethyl)pyrimidin-2-yl)amino)piperidine-1-sulfonyl chloride (14 mg, 0.04 mmol, 1.00 eq.) in DMF (2.0 mL) was added to a stirred solution of 1-(1-methyl-6-(1-(3-(piperidin-4-yl)propyl)piperidin-4-yl)-1H-indazol-3-yl)dihydropyrimidine-2,4(1H,3H)-dione (19 mg, 0.04 mmol, 1.05 eq.) and TEA (8 mg, 0.08 mmol, 2.00 eq.) in DMF (2.0 mL) at 0° C. The mixture was stirred at RT for 12 h, then quenched with H₂O and extracted with DCM. The combined organic layer was washed with water, dried over anhydrous Na₂SO₄, filtered, and concentrated. The residue was purified by silica gel column chromatography, eluted with DCM/MeOH (20:1), to afford the title compound as white solid. MS (ES, m/z): [M+1]⁺=761.1.

### Example 29

### Synthesis of 1-(6-(4-(3-((4-((5-chloropyrimidin-2-yl)amino)piperidin-1-yl)sulfonyl)benzyl)-piperazin-1-yl)-1-methyl-1H-indazol-3-yl)dihydropyrimidine-2,4(1H,3H)-dione

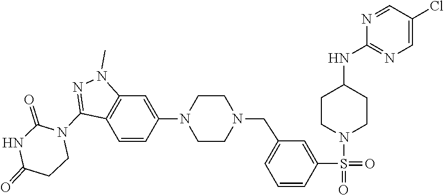 [SMILES: Cn1nc(N2CCC(=O)NC2=O)c2ccc(N3CCN(Cc4cccc(S(=O)(=O)N5CCC(Nc6ncc(Cl)cn6)CC5)c4)CC3)cc21]

### Step 1: tert-Butyl 4-((5-chloropyrimidin-2-yl)amino)piperidine-1-carboxylate

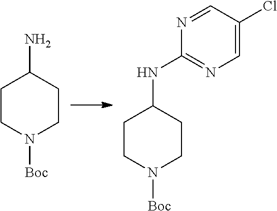 [SMILES: CC(C)(C)OC(=O)N1CCC(N)CC1 ; CC(C)(C)OC(=O)N1CCC(Nc2ncc(Cl)cn2)CC1]

A mixture of tert-butyl 4-aminopiperidine-1-carboxylate (3.50 g, 23.49 mmol, 1.00 eq.), 2,5-dichloropyrimidine (5.17 g, 25.84 mmol, 1.10 eq.) and DIEA (6.06 g, 46.98 mmol, 2.00 eq.) in DMSO (50.0 mL) was stirred at 90° C. under N₂ for 3 h. The mixture was diluted with water and extracted with EA. The organic layer was washed with brine, dried over Na₂SO₄, concentrated and the residue was purified by silica gel column chromatography eluting with PE/EtOAc (8:1) to give the title compound as white solid.

### Step 2: 5-Chloro-N-(piperidin-4-yl)pyrimidin-2-amine 2,2,2-trifluoroacetate

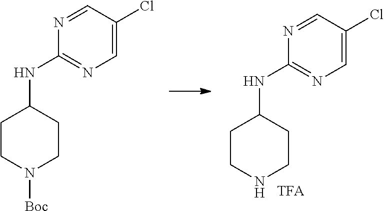 [SMILES: CC(C)(C)OC(=O)N1CCC(Nc2ncc(Cl)cn2)CC1 ; Clc1cnc(NC2CCNCC2)nc1]

A mixture of tert-butyl 4-((5-chloropyrimidin-2-yl)amino)piperidine-1-carboxylate (500 mg, 1.60 mmol, 1.00 eq.) and TFA (0.5 mL) in DCM (2.0 mL) was stirred at RT for 2 h. The mixture was concentrated to give the title compound as yellow oil.

### Step 3: N-(1-((3-(Bromomethyl)phenyl)sulfonyl)piperidin-4-yl)-5-chloropyrimidin-2-amine

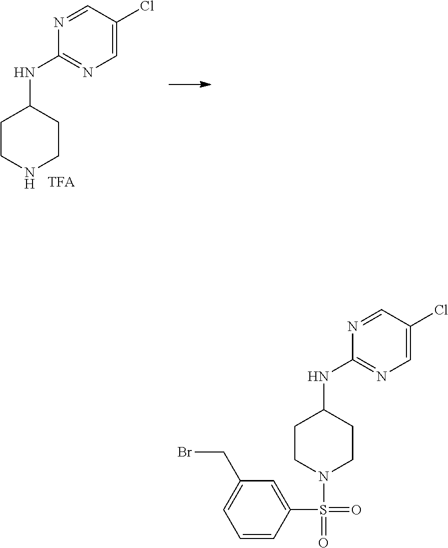 [SMILES: Clc1cnc(NC2CCNCC2)nc1 ; O=S(=O)(c1cccc(CBr)c1)N1CCC(Nc2ncc(Cl)cn2)CC1]

3-(Bromomethyl)benzenesulfonyl chloride (412 mg, 1.53 mmol, 1.00 eq.) in DCM (3.0 mL) was added to a stirred solution of 5-chloro-N-(piperidin-4-yl)pyrimidin-2-amine 2,2,2-trifluoroacetate (500 mg, 1.53 mmol, 1.00 eq.) and TEA (773 mg, 7.65 mmol, 5.00 eq.) in DCM (5.0 mL) slowly at −10° C. The mixture was warmed slowly to RT and stirred for 2 h. The mixture was quenched with H₂O and extracted with EA. The organic layer was washed with brine, dried over Na₂SO₄, concentrated and the residue was purified by silica gel column chromatography eluting with PE/EtOAc (5:1) to give the title compound as white solid.

### Step 4: 1-(6-(4-(3-((4-((5-Chloropyrimidin-2-yl)amino)piperidin-1-yl)sulfonyl)benzyl)piperazin-1-yl)-1-methyl-1H-indazol-3-yl)dihydropyrimidine-2,4(1H,3H)-dione

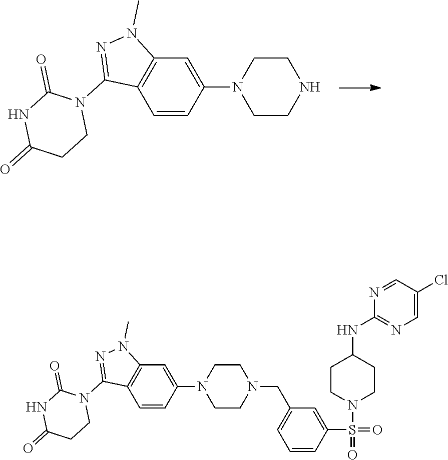 [SMILES: Cn1nc(N2CCC(=O)NC2=O)c2ccc(N3CCNCC3)cc21 ; Cn1nc(N2CCC(=O)NC2=O)c2ccc(N3CCN(Cc4cccc(S(=O)(=O)N5CCC(Nc6ncc(Cl)cn6)CC5)c4)CC3)cc21]

A mixture of 1-(1-methyl-6-(piperazin-1-yl)-1H-indazol-3-yl)dihydropyrimidine-2,4(1H,3H)-dione (50 mg, 0.15 mmol, 1.00 eq.), N-(1-((3-(bromomethyl)phenyl)sulfonyl)-piperidin-4-yl)-5-chloropyrimidin-2-amine (102 mg, 0.23 mmol, 1.50 eq.), and TEA (45 mg, 0.45 mmol, 3.00 eq.) in DMF (2.0 mL) was stirred at 60° C. under N₂ overnight. The mixture was diluted with EA, filtered and concentrated to give the crude product. The crude product was purified by prep-TLC to give the title compound as white solid. MS (ES, m/z): [M+1]+=693.0.

The following compounds were synthesized by proceeding analogously as described in Example 29.

| Ex# | Structure | Synthesis |
|---|---|---|
| 30 | 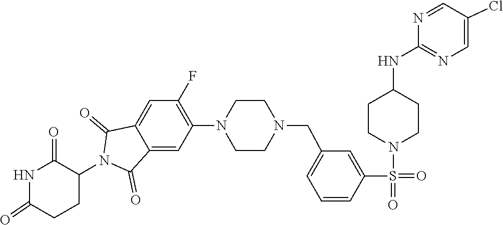 [SMILES: O=C1CCC(N2C(=O)c3cc(F)c(N4CCN(Cc5cccc(S(=O)(=O)N6CCC(Nc7ncc(Cl)cn7)CC6)c5)CC4)cc3C2=O)C(=O)N1] | 2-(2,6-dioxopiperidin-3-yl)- 5-fluoro-6-(piperazin-1-yl)- isoindoline-1,3-dione replaced 1-(1-methyl-6-(piperazin-1- yl)-1H-indazol-3-yl)dihydro- pyrimidine-2,4(1H,3H)-dione in Step 4 |
| 31 | 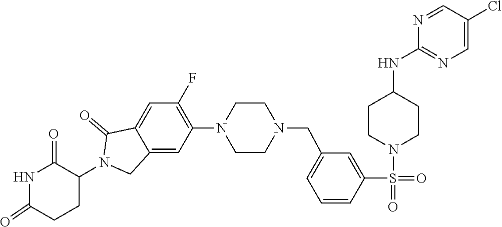 [SMILES: O=C1CCC(N2Cc3cc(N4CCN(Cc5cccc(S(=O)(=O)N6CCC(Nc7ncc(Cl)cn7)CC6)c5)CC4)c(F)cc3C2=O)C(=O)N1] | 3-(6-fluoro-1-oxo-5-(piper- azin-1-yl)isoindolin-2-yl)- piperidine-2,6-dione replaced 1-(1-methyl-6-(piperazin-1- yl)-1H-indazol-3-yl)dihydro- pyrimidine-2,4(1H,3H)-dione in Step 4 |
| 32 | 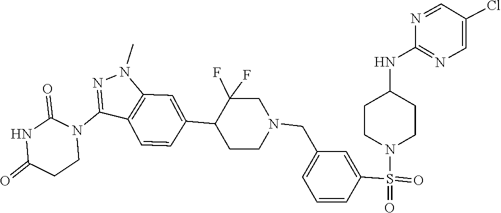 [SMILES: Cn1nc(N2CCC(=O)NC2=O)c2ccc(C3CCN(Cc4cccc(S(=O)(=O)N5CCC(Nc6ncc(Cl)cn6)CC5)c4)CC3(F)F)cc21] | Ref. 53 replaced 1-(1-methyl- 6-(piperazin-1-yl)-1H-inda- zol-3-yl)dihydropyrimidine- 2,4(1H,3H)-dione in Step 4 |
| 33 | 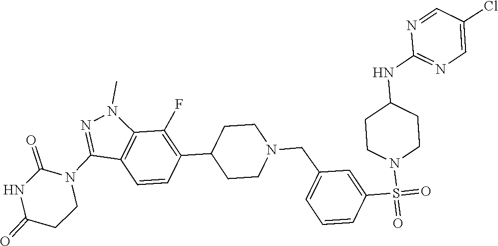 [SMILES: Cn1nc(N2CCC(=O)NC2=O)c2ccc(C3CCN(Cc4cccc(S(=O)(=O)N5CCC(Nc6ncc(Cl)cn6)CC5)c4)CC3)c(F)c21] | Ref. 45a replaced 1-(1-meth- yl-6-(piperazin-1-yl)-1H- indazol-3-yl)dihydropyrim- idine-2,4(1H,3H)-dione in Step 4 |
| 34 | 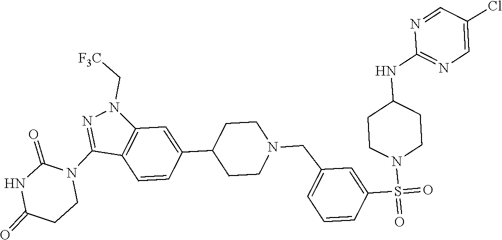 [SMILES: O=C1CCN(c2nn(CC(F)(F)F)c3cc(C4CCN(Cc5cccc(S(=O)(=O)N6CCC(Nc7ncc(Cl)cn7)CC6)c5)CC4)ccc23)C(=O)N1] | Ref. 50 replaced 1-(1-methyl- 6-(piperazin-1-yl)-1H-inda- zol-3-yl)dihydropyrimidine- 2,4(1H,3H)-dione in Step 4 |
| 35 | 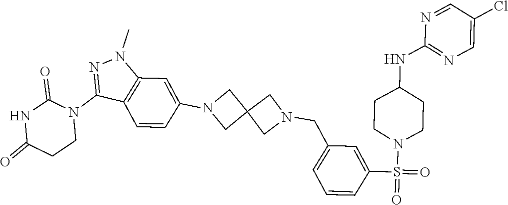 [SMILES: Cn1nc(N2CCC(=O)NC2=O)c2ccc(N3CC4(CN(Cc5cccc(S(=O)(=O)N6CCC(Nc7ncc(Cl)cn7)CC6)c5)C4)C3)cc21] | Ref. 57 replaced 1-(1-methyl- 6-(piperazin-1-yl)-1H-inda- zol-3-yl)dihydropyrimidine- 2,4(1H,3H)-dione in Step 4 |

### Example 36

### Synthesis of 1-(1-methyl-6-(1-(2-(1-((4-((5-(trifluoromethyl)pyrimidin-2-yl)amino)piperidin-1-yl)sulfonyl)piperidin-3-yl)ethyl)piperidin-4-yl)-1H-indazol-3-yl)dihydropyrimidine-2,4(1H,3H)-dione

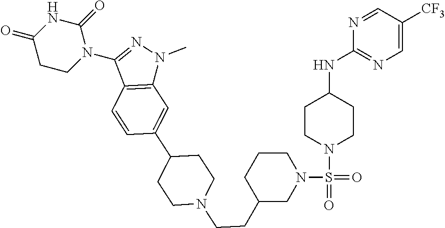 [SMILES: Cn1nc(N2CCC(=O)NC2=O)c2ccc(C3CCN(CCC4CCCN(S(=O)(=O)N5CCC(Nc6ncc(C(F)(F)F)cn6)CC5)C4)CC3)cc21]

### Step 1: Methyl 2-(1-(chlorosulfonyl)piperidin-3-yl)acetate

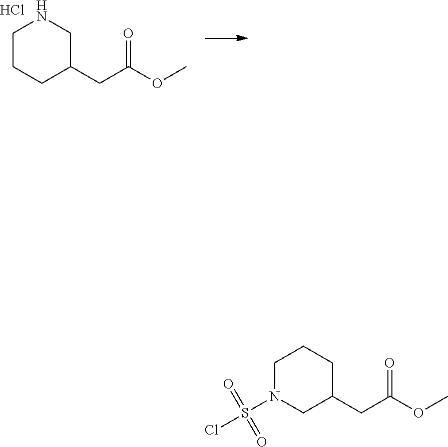

SO₂Cl₂ (836.2 mg, 6.20 mol, 1.20 eq.) in DCM (5.0 mL) was added to a stirred solution of methyl 2-(piperidin-3-yl)acetate hydrochloride (1.00 g, 5.16 mmol, 1.00 eq.) and TEA (781.7 mg, 7.74 mmol, 1.50 eq.) in DCM (5.00 mL) dropwise at 0° C. The mixture was stirred at r.t for 12 h and then diluted with water and extracted with DCM. The combined organic layer was washed with brine, dried over anhydrous Na₂SO₄ and concentrated and purified by silica gel column chromatography, eluted with PE/EA=3:1, to afford the title compound as yellow oil.

### Step 2: Methyl 2-(1-((4-((5-(trifluoromethyl)pyrimidin-2-yl)amino)piperidin-1-yl)sulfonyl)-piperidin-3-yl)acetate

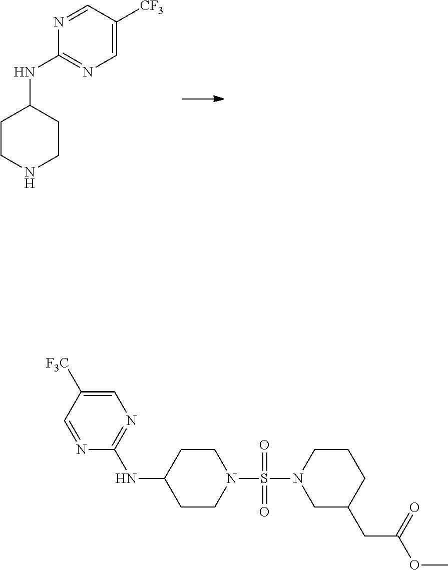 [SMILES: FC(F)(F)c1cnc(NC2CCNCC2)nc1 ; COC(=O)CC1CCCN(S(=O)(=O)N2CCC(Nc3ncc(C(F)(F)F)cn3)CC2)C1]

Methyl 2-(1-(chlorosulfonyl)piperidin-3-yl)acetate (228 mg, 0.896 mmol, 1.00 eq.) in DCM (5.0 mL) was added to a stirred solution of N-(piperidin-4-yl)-5-(trifluoromethyl)pyrimidin-2-amine 2,2,2-trifluoroacetate (220 mg, 0.896 mmol, 1.00 eq.) and TEA (271 mg, 2.69 mmol, 3.00 eq.) in DCM (5.0 mL) dropwise at 0° C. The mixture was stirred at RT for 2 h, quenched with water and extracted with DCM. The combined organic layer was washed with water, dried over anhydrous Na₂SO₄, filtered, and concentrated. The residue was purified by silica gel column chromatography, eluted with PE/EA (3:1), to afford the title compound as yellow solid.

### Step 3: 2-(1-((4-((5-(Trifluoromethyl)pyrimidin-2-yl)amino)piperidin-1-yl)sulfonyl)piperidin-3-yl) ethanol

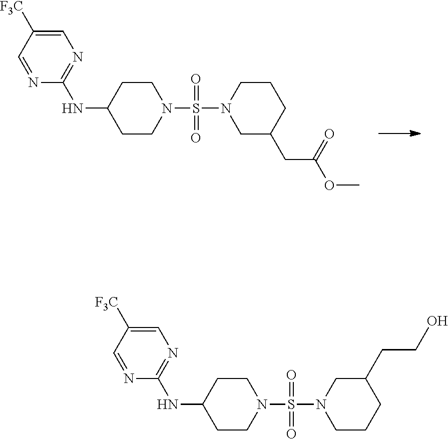 [SMILES: COC(=O)CC1CCCN(S(=O)(=O)N2CCC(Nc3ncc(C(F)(F)F)cn3)CC2)C1 ; O=S(=O)(N1CCC(Nc2ncc(C(F)(F)F)cn2)CC1)N1CCCC(CCO)C1]

Methyl 2-(1-((4-((5-(trifluoromethyl)pyrimidin-2-yl)amino)piperidin-1-yl)sulfonyl)piperidin-3-yl)acetate (320 mg, 0.688 mmol, 1.00 eq.) in THF (5.0 mL) was added to a stirred solution of LiAlH₄ (52 mg, 1.38 mmol, 2.00 eq.) in THF (5.0 mL) dropwise at 0° C. The mixture was stirred at RT for 12 h, quenched with water, and then extracted with EA. The combined organic layer was washed with water, dried over anhydrous Na₂SO₄, filtered, and then concentrated. The residue was purified by silica gel column chromatography, eluted with PE/EA (3:1), to afford the title compound as white solid.

### Step 4: 2-(1-((4-((5-(Trifluoromethyl)pyrimidin-2-yl)amino)piperidin-1-yl)sulfonyl)piperidin-3-yl)acetaldehyde

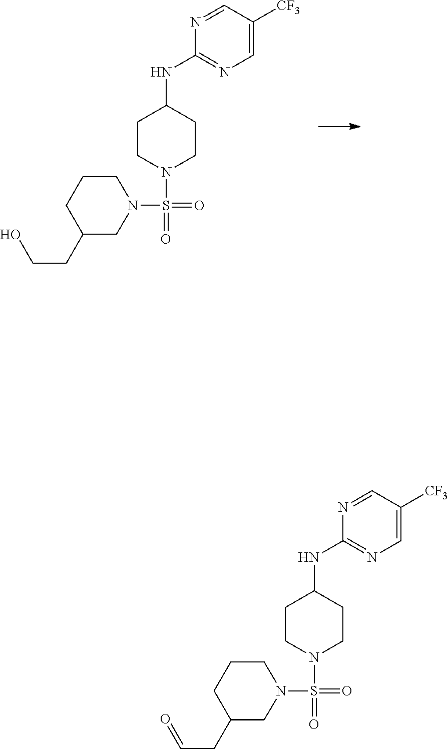 [SMILES: O=S(=O)(N1CCC(Nc2ncc(C(F)(F)F)cn2)CC1)N1CCCC(CCO)C1 ; O=CCC1CCCN(S(=O)(=O)N2CCC(Nc3ncc(C(F)(F)F)cn3)CC2)C1]

To a stirred solution of 2-(1-((4-((5-(trifluoromethyl)pyrimidin-2-yl)amino)piperidin-1-yl)sulfonyl)piperidin-3-yl) ethanol (80 mg, 0.183 mmol, 1.00 eq.) in DCM (5.0 mL) was added Dess-Martin periodinane (116 mg, 0.274 mmol, 1.50 eq.) at 0° C. The mixture was stirred at RT for 2 h, quenched with water, and then extracted with EA. The combined organic layer was washed with water, dried over anhydrous Na₂SO₄, filtered, and then concentrated. The residue was purified by silica gel column chromatography, eluted with PE/EA (3:1), to afford the title compound as a white oil.

### Step 5: 1-(1-Methyl-6-(1-(2-(1-((4-((5-(trifluoromethyl)pyrimidin-2-yl)amino)piperidin-1-yl)-sulfonyl)piperidin-3-yl)ethyl)piperidin-4-yl)-1H-indazol-3-yl)dihydropyrimidine-2,4(1H,3H)-dione

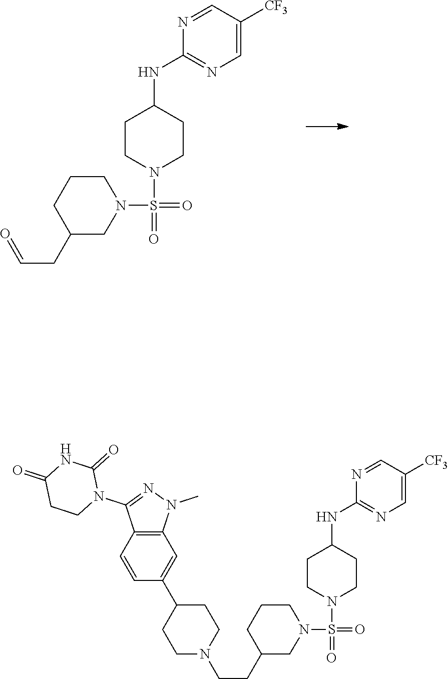 [SMILES: O=CCC1CCCN(S(=O)(=O)N2CCC(Nc3ncc(C(F)(F)F)cn3)CC2)C1 ; Cn1nc(N2CCC(=O)NC2=O)c2ccc(C3CCN(CCC4CCCN(S(=O)(=O)N5CCC(Nc6ncc(C(F)(F)F)cn6)CC5)C4)CC3)cc21]

A mixture of 1-(1-methyl-6-(piperidin-4-yl)-1H-indazol-3-yl)dihydropyrimidine-2,4(1H,3H)-dione trifluoroacetate (77 mg, 0.234 mmol, 1.00 eq.), CH₃COOH (1 drops) and 2-(1-((4-((5-(trifluoromethyl)pyrimidin-2-yl)amino)piperidin-1-yl)sulfonyl)piperidin-3-yl)acetaldehyde (102 mg, 0.234 mmol, 1.00 eq) in THF (2.0 mL)/DMF (2.0 mL) was stirred at 45° C. for 0.5 h. NaBH₃CN (29 mg, 0.468 mmol, 2.00 eq.) was added to the mixture at 0° C. and the mixture was stirred at RT for 12 h. The mixture was quenched with water and then extracted with EA. The combined organic layer was washed with water, dried over anhydrous Na₂SO₄, filtered, and then concentrated. The residue was purified by prep-TLC to give title compounds. MS (ES, m/z): [M+1]⁺=747.6.

### Example 37

### Synthesis of 1-(6-(1-((1-((3-fluoro-4-((5-(trifluoromethyl)pyrimidin-2-yl)amino)phenyl)-sulfonyl)piperidin-4-yl)methyl)piperidin-4-yl)-1-methyl-1H-indazol-3-yl)dihydropyrimidine-2,4(1H,3H)-dione

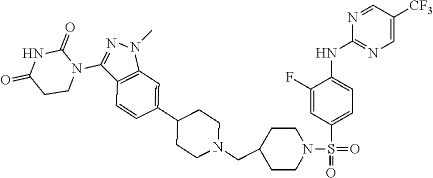 [SMILES: Cn1nc(N2CCC(=O)NC2=O)c2ccc(C3CCN(CC4CCN(S(=O)(=O)c5ccc(Nc6ncc(C(F)(F)F)cn6)c(F)c5)CC4)CC3)cc21]

### Step 1: tert-Butyl 4-((4-(3-(2,4-dioxotetrahydropyrimidin-1(2H)-yl)-1-methyl-1H-indazol-6-yl)piperidin-1-yl)methyl)piperidine-1-carboxylate

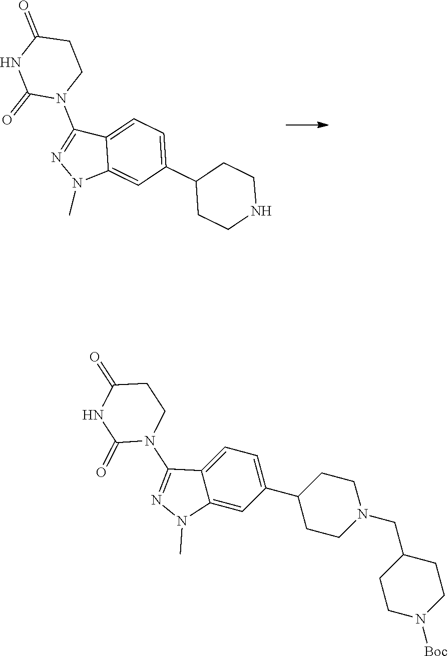 [SMILES: Cn1nc(N2CCC(=O)NC2=O)c2ccc(C3CCNCC3)cc21 ; Cn1nc(N2CCC(=O)NC2=O)c2ccc(C3CCN(CC4CCN(C(=O)OC(C)(C)C)CC4)CC3)cc21]

A mixture of 1-(1-methyl-6-(piperidin-4-yl)-1H-indazol-3-yl)dihydropyrimidine-2,4(1H,3H)-dione (500 mg, 1.40 mmol, 1.00 eq.), AcOH (3 drops), tert-butyl 4-formylpiperidine-1-carboxylate (300 mg, 1.40 mmol, 1.00 eq.) in THF (5.00 mL)/DMF (2.5 mL) was stirred at 45° C. for 45 min. The solution was cooled to r.t and NaBH₃CN (168 mg, 2.80 mmol, 2.00 eq.) was added. The mixture was stirred at r.t for 2 h, and then diluted with water and extracted with EtOAc. The combined organic layer was washed with brine, dried over anhydrous Na₂SO₄ and concentrated. The residue was purified by silica gel chromatography, eluted with DCM/MeOH (20:1), to afford the title compound as a white solid.

### Step 2: 1-(1-Methyl-6-(1-(piperidin-4-ylmethyl)piperidin-4-yl)-1H-indazol-3-yl)dihydro-pyrimidine-2,4(1H,3H)-dione

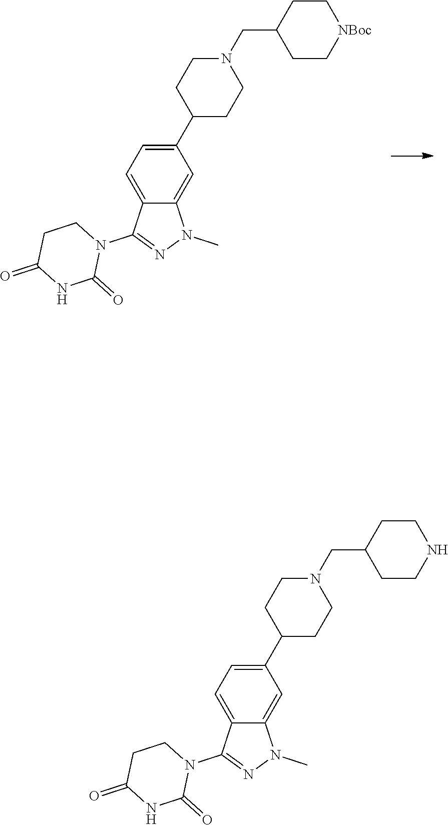 [SMILES: Cn1nc(N2CCC(=O)NC2=O)c2ccc(C3CCN(CC4CCN(C(=O)OC(C)(C)C)CC4)CC3)cc21 ; Cn1nc(N2CCC(=O)NC2=O)c2ccc(C3CCN(CC4CCNCC4)CC3)cc21]

To a stirred solution of tert-butyl 4-((4-(3-(2,4-dioxotetrahydropyrimidin-1(2H)-yl)-1-methyl-1H-indazol-6-yl)piperidin-1-yl)methyl)piperidine-1-carboxylate (346 mg, 0.66 mmol, 1.00 eq.) in TFA/DCM (0.5 mL/2.0 mL) was stirred at rt for 2 h. The mixture was concentrated to give the title compound as brown oil.

### Step 3: 1-(6-(1-((1-((3-Fluoro-4-nitrophenyl)sulfonyl)piperidin-4-yl)methyl)piperidin-4-yl)-1-methyl-1H-indazol-3-yl)dihydropyrimidine-2,4(1H,3H)-dione

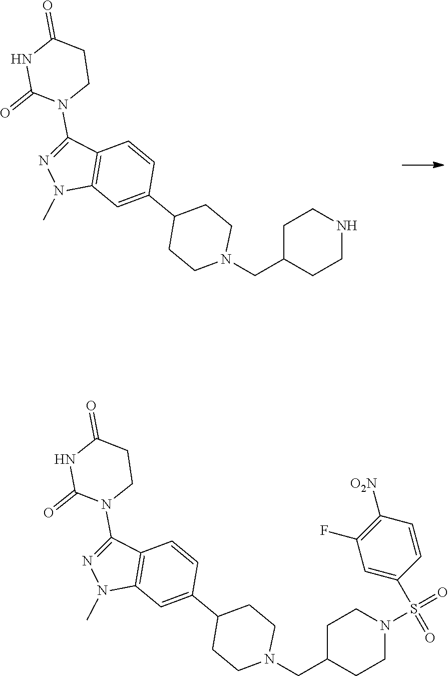 [SMILES: Cn1nc(N2CCC(=O)NC2=O)c2ccc(C3CCN(CC4CCNCC4)CC3)cc21 ; Cn1nc(N2CCC(=O)NC2=O)c2ccc(C3CCN(CC4CCN(S(=O)(=O)c5ccc([N+](=O)[O-])c(F)c5)CC4)CC3)cc21]

3-Fluoro-4-nitrobenzenesulfonyl chloride (54 mg, 0.23 mmol, 1.20 eq.) was added to a stirred solution of 1-(1-methyl-6-(1-(piperidin-4-ylmethyl)piperidin-4-yl)-1H-indazol-3-yl)dihydropyrimidine-2,4(1H,3H)-dione (80 mg, 0.19 mmol, 1.00 eq.) and TEA (57 mg, 0.57 mmol, 3.00 eq.) in DCM (3.0 mL) at 0° C. The mixture was stirred at RT for 2 h, quenched with H₂O and extracted with DCM. The combined organic layer was washed with water, dried over anhydrous Na₂SO₄, filtered, and concentrated. The residue was purified by silica gel column chromatography, eluted with DCM/MeOH (20:1), to afford the title compound as white solid.

### Step 4: 1-(6-(1-((1-((4-Amino-3-fluorophenyl)sulfonyl)piperidin-4-yl)methyl)piperidin-4-yl)-1-methyl-1H-indazol-3-yl)dihydropyrimidine-2,4(1H,3H)-dione

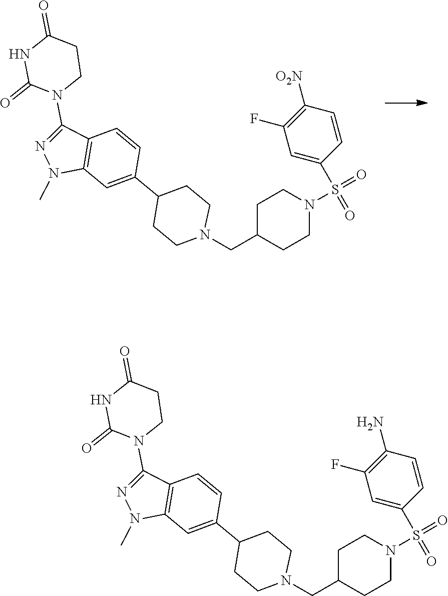 [SMILES: Cn1nc(N2CCC(=O)NC2=O)c2ccc(C3CCN(CC4CCN(S(=O)(=O)c5ccc([N+](=O)[O-])c(F)c5)CC4)CC3)cc21 ; Cn1nc(N2CCC(=O)NC2=O)c2ccc(C3CCN(CC4CCN(S(=O)(=O)c5ccc(N)c(F)c5)CC4)CC3)cc21]

A mixture of 1-(6-(1-((1-((3-fluoro-4-nitrophenyl)sulfonyl)piperidin-4-yl)methyl)-piperidin-4-yl)-1-methyl-1H-indazol-3-yl)dihydropyrimidine-2,4(1H,3H)-dione (65 mg, 0.09 mmol, 1.00 eq.) and Pd/C (10% w/w, 33 mg) in MeOH (2.0 mL) was stirred at r.t. for 12 h under H₂ atmosphere. The mixture was filtered, and the filtrate was concentrated to give the title compound as yellow solid.

### Step 5: 1-(6-(1-((1-((3-Fluoro-4-((5-(trifluoromethyl)pyrimidin-2-yl)amino)phenyl)sulfonyl)-piperidin-4-yl)methyl)piperidin-4-yl)-1-methyl-1H-indazol-3-yl)dihydropyrimidine-2,4(1H,3H)-dione

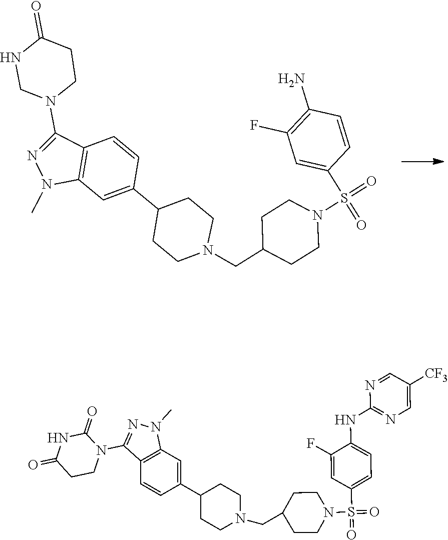 [SMILES: Cn1nc(N2CCC(=O)NC2)c2ccc(C3CCN(CC4CCN(S(=O)(=O)c5ccc(N)c(F)c5)CC4)CC3)cc21 ; Cn1nc(N2CCC(=O)NC2=O)c2ccc(C3CCN(CC4CCN(S(=O)(=O)c5ccc(Nc6ncc(C(F)(F)F)cn6)c(F)c5)CC4)CC3)cc21]

A solution of 1-(6-(1-((1-((4-amino-3-fluorophenyl)sulfonyl)piperidin-4-yl)methyl)piperidin-4-yl)-1-methyl-1H-indazol-3-yl)dihydropyrimidine-2,4(1H,3H)-dione (30 mg, 0.05 mmol, 1.00 eq.), 2-chloro-5-(trifluoromethyl)pyrimidine (14 mg, 0.08 mmol, 1.50 eq.), Pd₂(dba)₃ (5 mg, 0.005 mmol, 0.10 eq.), XantPhos (6 mg, 0.01 mmol, 0.02 eq.) and K₃PO₄ (21 mg, 0.10 mmol, 2.00 eq) in dioxane (2.0 mL) was stirred at 100° C. for 12 h under N₂. The resulting mixture was diluted with water and extracted with EtOAc. The combined organic layer was washed with brine, dried over anhydrous Na₂SO₄ and concentrated. The residue was purified by prep-TLC (DCM:MeOH=20:1) to give the title compound as a white solid. MS (ES, m/z): [M+1]⁺=744.0.

### Example 38

### Synthesis of 1-(1-methyl-6-(1-(3-((4-((5-vinylpyrimidin-2-yl)amino)piperidin-1-yl)sulfonyl)-benzyl)piperidin-4-yl)-1H-indazol-3-yl)dihydropyrimidine-2,4(1H,3H)-dione

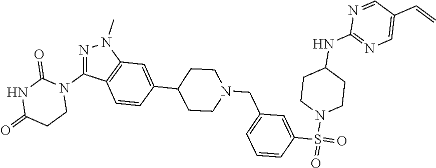 [SMILES: C=Cc1cnc(NC2CCN(S(=O)(=O)c3cccc(CN4CCC(c5ccc6c(N7CCC(=O)NC7=O)nn(C)c6c5)CC4)c3)CC2)nc1]

A mixture of 1-(6-(1-(3-((4-aminopiperidin-1-yl)sulfonyl)benzyl)piperidin-4-yl)-1-methyl-1H-indazol-3-yl)dihydropyrimidine-2,4(1H,3H)-dione (50 mg, 0.07 mmol, 1.00 eq.), Cs₂CO₃ (70 mg, 0.22 mmol, 3.00 eq.), 2-chloro-5-vinylpyrimidine (13 mg, 0.09 mmol, 1.20 eq.), Ruphos (20 mg) and Ruphos-Pd G2 (20 mg) in t-BuOH/NMP (2.0 mL/1.0 mL) was stirred at 90° C. under N₂ overnight. The mixture was diluted with water, extracted with DCM. The combined organic layers were dried over Na₂SO₄, concentrated and the residue was purified by prep-TLC to give the title compound as yellow solid. MS (ES, m/z): [M+1]+=684.1.

### Example 39

### Synthesis of 1-(1-methyl-6-(1-(3-(((1s,4s)-4-((5-(trifluoromethyl)pyrimidin-2-yl)amino) cyclohexyl)sulfonyl)benzyl)piperidin-4-yl)-1H-indazol-3-yl)dihydropyrimidine-2,4(1H,3H)-dione

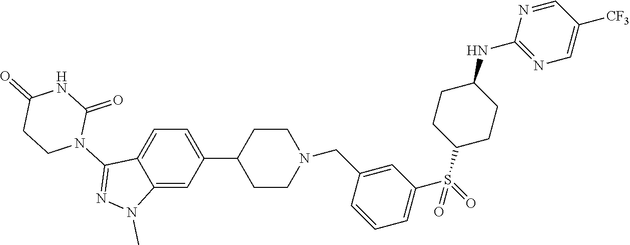

To a stirred mixture of 2-chloro-5-(trifluoromethyl)pyrimidine (52.7 mg, 0.30 mmol, 2.0 eq) and 1-(6-(1-(3-(((1s,4s)-4-aminocyclohexyl)sulfonyl)benzyl)piperidin-4-yl)-1-methyl-1H-indazol-3-yl)dihydropyrimidine-2,4(1H,3H)-dione 2,2,2-trifluoroacetate (Ref 58; 0.15 mmol, 1.0 eq) in anhydrous DMSO (2 mL) at 25° C. was added DIPEA (99.8 mg, 0.75 mmol, 5.0 eq). The resulting mixture was stirred at 80° C. under N₂ for 2 h. The mixture was diluted with water, and extracted with DCM. The combined organic layers were dried over Na₂SO₄, concentrated and the residue was purified by C18 column chromatography, eluted with MeCN/water (0-45%, 0.5% NH₄CO₃ in water) to give the title compound as a white solid. LCMS (ESI) m/z [M+H]⁺=725.3.

The following compounds were prepared by proceeding analogously as described in Example 39.

| Ex# | Structure | Synthesis |
|---|---|---|
| 40 | 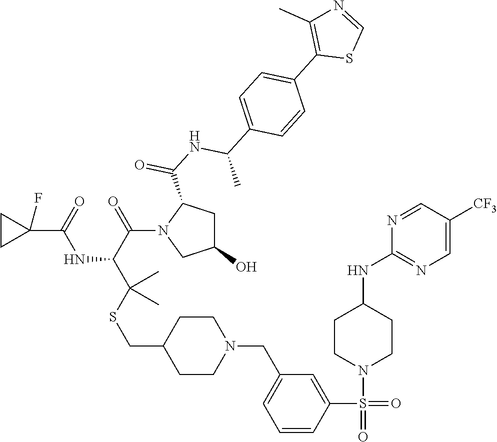 [SMILES: Cc1ncsc1-c1ccc([C@H](C)NC(=O)[C@@H]2C[C@@H](O)CN2C(=O)[C@@H](NC(=O)C2(F)CC2)C(C)(C)SCC2CCN(Cc3cccc(S(=O)(=O)N4CCC(Nc5ncc(C(F)(F)F)cn5)CC4)c3)CC2)cc1] | Ref. 59 replaced Ref. 58 |
| 41 | 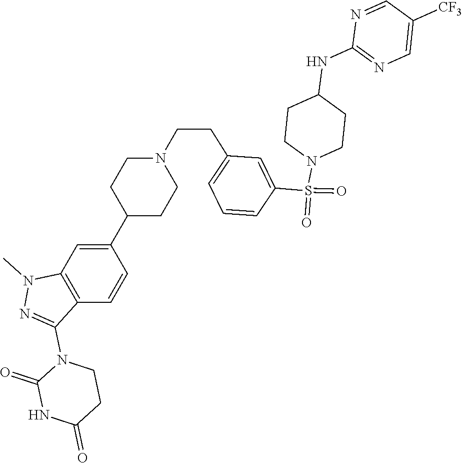 [SMILES: Cn1nc(N2CCC(=O)NC2=O)c2ccc(C3CCN(CCc4cccc(S(=O)(=O)N5CCC(Nc6ncc(C(F)(F)F)cn6)CC5)c4)CC3)cc21] | Ref. 60 replaced Ref. 58 |
| 42 | 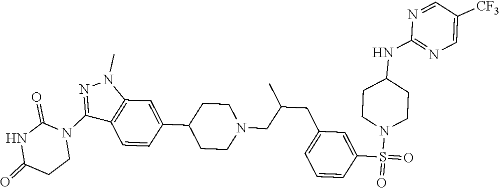 [SMILES: CC(Cc1cccc(S(=O)(=O)N2CCC(Nc3ncc(C(F)(F)F)cn3)CC2)c1)CN1CCC(c2ccc3c(N4CCC(=O)NC4=O)nn(C)c3c2)CC1] | Ref 61. replaced Ref. 58 |
| 43 | 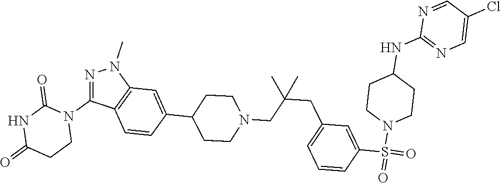 [SMILES: Cn1nc(N2CCC(=O)NC2=O)c2ccc(C3CCN(CC(C)(C)Cc4cccc(S(=O)(=O)N5CCC(Nc6ncc(Cl)cn6)CC5)c4)CC3)cc21] | Ref. 62 replaced Ref. 58 and 2,5-dichloropyrim- idine replaced 2-chloro- 5-(trifluoromethyl)pyr- imidine |

### Example 44

### Synthesis of N—((R)-1-((2S,4R)-4-hydroxy-2-(((S)-1-(4-(4-methylthiazol-5-yl)phenyl)ethyl)-carbamoyl) pyrrolidin-1-yl)-3,3-dimethyl-1-oxobutan-2-yl)-1-(3-((4-((5-(trifluoromethyl)-pyrimidin-2-yl)amino)piperidin-1-yl)sulfonyl)benzyl)piperidine-4-carboxamide

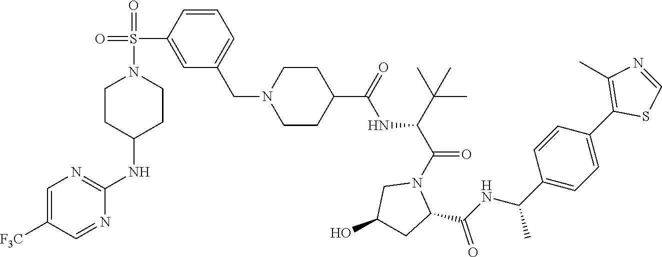 [SMILES: Cc1ncsc1-c1ccc([C@H](C)NC(=O)[C@@H]2C[C@@H](O)CN2C(=O)[C@H](NC(=O)C2CCN(Cc3cccc(S(=O)(=O)N4CCC(Nc5ncc(C(F)(F)F)cn5)CC4)c3)CC2)C(C)(C)C)cc1]

### Step 1: tert-Butyl 4-(((R)-1-((2S,4R)-4-hydroxy-2-(((S)-1-(4-(4-methylthiazol-5-yl)phenyl)ethyl) carbamoyl) pyrrolidin-1-yl)-3,3-dimethyl-1-oxobutan-2-yl) carbamoyl)piperidine-1-carboxylate

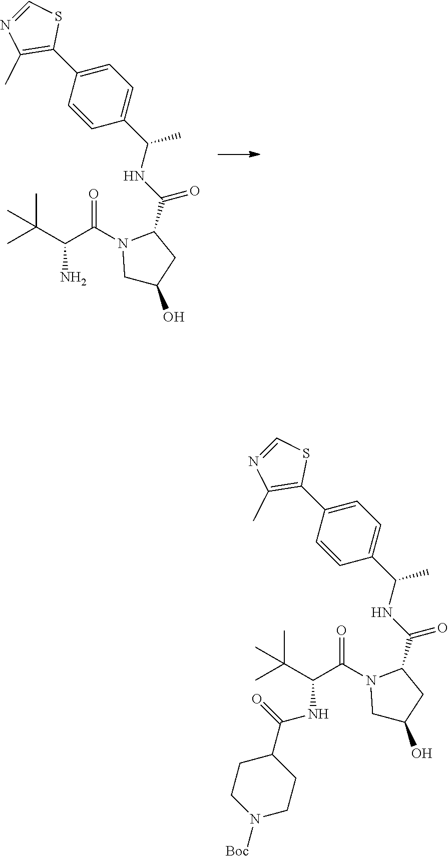 [SMILES: Cc1ncsc1-c1ccc([C@H](C)NC(=O)[C@@H]2C[C@@H](O)CN2C(=O)[C@H](N)C(C)(C)C)cc1 ; Cc1ncsc1-c1ccc([C@H](C)NC(=O)[C@@H]2C[C@@H](O)CN2C(=O)[C@H](NC(=O)C2CCN(C(=O)OC(C)(C)C)CC2)C(C)(C)C)cc1]

To a stirred solution of (2S,4R)-1-[(2R)-2-amino-3,3-dimethyl-butanoyl]-4-hydroxy-N-[(1S)-1-[4-(4-methylthiazol-5-yl)phenyl]ethyl]pyrrolidine-2-carboxamide (890 mg, 2 mmol) and 1-tert-butoxycarbonylpiperidine-4-carboxylic acid (920 mg, 4 mmol) in 3 mL DMF was added HATU (910 mg, 2.4 mmol) and N,N-diisopropylethylamine (780 mg, 6 mmol), and the mixture was stirred for 2 h at rt. The mixture was quenched by water, extracted by EtOAc, the organic layer was dried over Na₂SO₄, filtered, and concentrated. The residue was purified by flash column chromatography eluted with MeOH/DCM=0-6%, to afford the title compound as yellow solid.

### Step 2: N—((R)-1-((2S,4R)-4-hydroxy-2-(((S)-1-(4-(4-methylthiazol-5-yl)phenyl)ethyl) carbamoyl) pyrrolidin-1-yl)-3,3-dimethyl-1-oxobutan-2-yl)piperidine-4-carboxamide

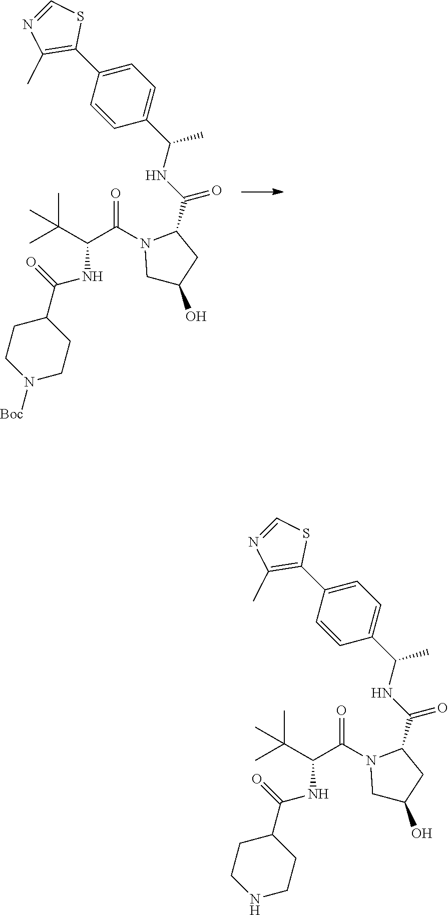 [SMILES: Cc1ncsc1-c1ccc([C@H](C)NC(=O)[C@@H]2C[C@@H](O)CN2C(=O)[C@H](NC(=O)C2CCN(C(=O)OC(C)(C)C)CC2)C(C)(C)C)cc1 ; Cc1ncsc1-c1ccc([C@H](C)NC(=O)[C@@H]2C[C@@H](O)CN2C(=O)[C@H](NC(=O)C2CCNCC2)C(C)(C)C)cc1]

A mixture of tert-butyl 4-[[(1R)-1-[(2S,4R)-4-hydroxy-2-[[(1S)-1-[4-(4-methylthiazol-5-yl)-phenyl]ethyl]carbamoyl]pyrrolidine-1-carbonyl]-2,2-dimethyl-propyl]carbamoyl]piperidine-1-carboxylate (1.3 g, 1.98 mmol) in 4 mL 2M HCl in EA was stirred 30 min at rt. The mixture was concentrated, and the residue was basified by addition of NH₃/MeOH then concentrated to afford title compound as yellow solid.

### Step 3: tert-Butyl (1-((3-((4-(((R)-1-((2S,4R)-4-hydroxy-2-(((S)-1-(4-(4-methylthiazol-5-yl)phenyl)ethyl) carbamoyl) pyrrolidin-1-yl)-3,3-dimethyl-1-oxobutan-2-yl) carbamoyl)piperidin-1-yl)methyl)phenyl)sulfonyl)piperidin-4-yl)carbamate

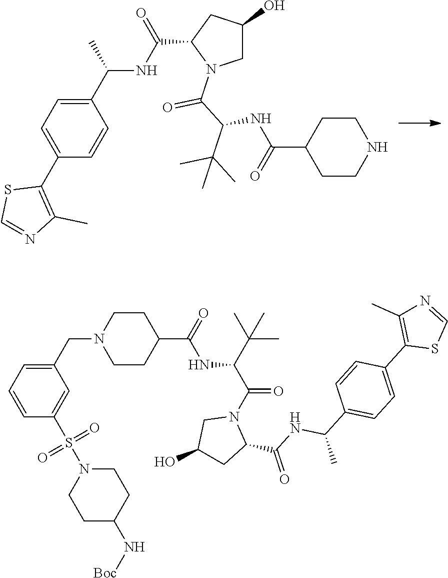 [SMILES: Cc1ncsc1-c1ccc([C@H](C)NC(=O)[C@@H]2C[C@@H](O)CN2C(=O)[C@H](NC(=O)C2CCNCC2)C(C)(C)C)cc1 ; Cc1ncsc1-c1ccc([C@H](C)NC(=O)[C@@H]2C[C@@H](O)CN2C(=O)[C@H](NC(=O)C2CCN(Cc3cccc(S(=O)(=O)N4CCC(NC(=O)OC(C)(C)C)CC4)c3)CC2)C(C)(C)C)cc1]

A mixture of N-[(1R)-1-[(2S,4R)-4-hydroxy-2-[[(1S)-1-[4-(4-methylthiazol-5-yl)phenyl]-ethyl]carbamoyl]pyrrolidine-1-carbonyl]-2,2-dimethyl-propyl]piperidine-4-carboxamide (650 mg, 1.16 mmol) and tert-butyl N-[1-[3-(bromomethyl)phenyl]sulfonyl-4-piperidyl]carbamate (507 mg, 1.16 mmol), and N,N-diisopropylethylamine (453 mg, 3.51 mmol) in 2.5 mL DMF and 2.5 mL THF was stirred for 3 h at 50° C. The mixture was filtered, and the solid was purified by flash column chromatography, eluting with MeOH/DCM=0-10%, afford the title compound as yellow solid.

### Step 4: 1-(3-((4-Aminopiperidin-1-yl)sulfonyl)benzyl)-N—((R)-1-((2S,4R)-4-hydroxy-2-(((S)-1-(4-(4-methylthiazol-5-yl)phenyl)ethyl) carbamoyl) pyrrolidin-1-yl)-3,3-dimethyl-1-oxobutan-2-yl)piperidine-4-carboxamide

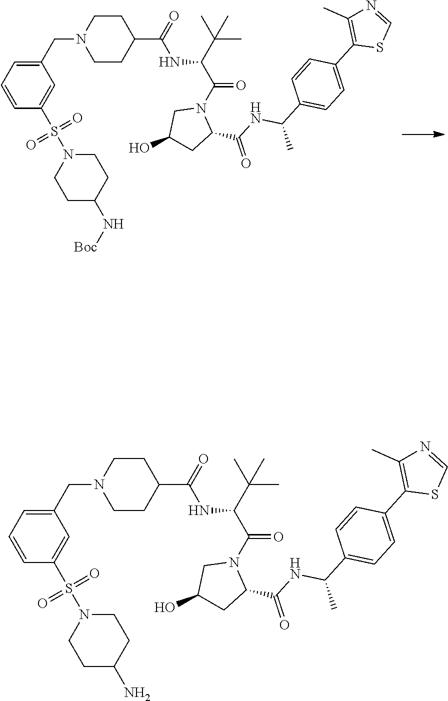 [SMILES: Cc1ncsc1-c1ccc([C@H](C)NC(=O)[C@@H]2C[C@@H](O)CN2C(=O)[C@H](NC(=O)C2CCN(Cc3cccc(S(=O)(=O)N4CCC(NC(=O)OC(C)(C)C)CC4)c3)CC2)C(C)(C)C)cc1 ; Cc1ncsc1-c1ccc([C@H](C)NC(=O)[C@@H]2C[C@@H](O)CN2C(=O)[C@H](NC(=O)C2CCN(Cc3cccc(S(=O)(=O)N4CCC(N)CC4)c3)CC2)C(C)(C)C)cc1]

A mixture of tert-butyl N-[1-[3-[[4-[[(1R)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethylpropyl]-carbamoyl]-1-piperidyl]methyl]phenyl]sulfonyl-4-piperidyl]carbamate (700 mg, 0.77 mmol) in 2 mL 2M HCl in EtOAc was stirred 30 min at rt. The mixture was concentrated, and the residue was basified by addition of NH₃/MeOH then concentrated to afford the title compound as yellow solid.

### Step 5: N—((R)-1-((2S,4R)-4-hydroxy-2-(((S)-1-(4-(4-methylthiazol-5-yl)phenyl)ethyl)-carbamoyl) pyrrolidin-1-yl)-3,3-dimethyl-1-oxobutan-2-yl)-1-(3-((4-((5-(trifluoromethyl)-pyrimidin-2-yl)amino)piperidin-1-yl)sulfonyl)benzyl)piperidine-4-carboxamide

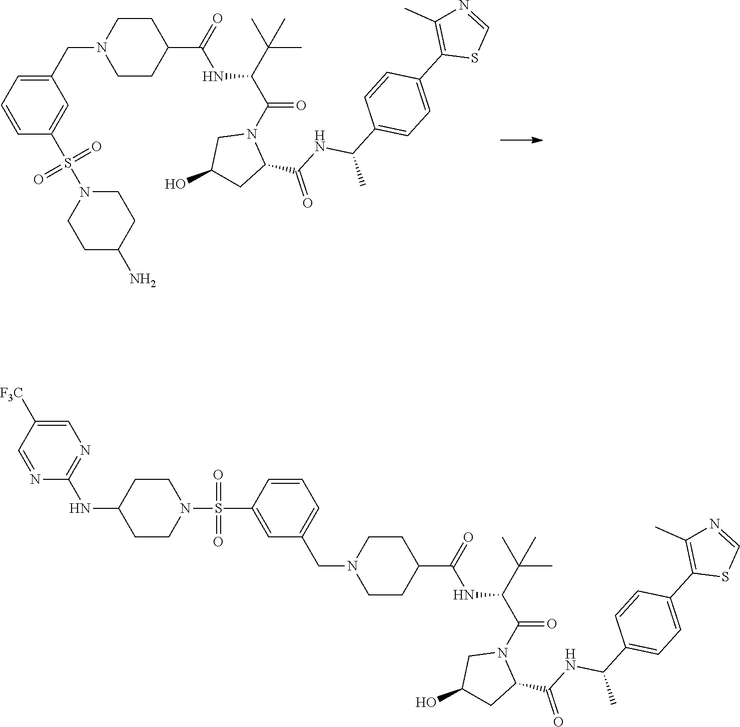 [SMILES: Cc1ncsc1-c1ccc([C@H](C)NC(=O)[C@@H]2C[C@@H](O)CN2C(=O)[C@H](NC(=O)C2CCN(Cc3cccc(S(=O)(=O)N4CCC(N)CC4)c3)CC2)C(C)(C)C)cc1 ; Cc1ncsc1-c1ccc([C@H](C)NC(=O)[C@@H]2C[C@@H](O)CN2C(=O)[C@H](NC(=O)C2CCN(Cc3cccc(S(=O)(=O)N4CCC(Nc5ncc(C(F)(F)F)cn5)CC4)c3)CC2)C(C)(C)C)cc1]

A mixture of 1-[[3-[(4-amino-1-piperidyl)sulfonyl]phenyl]methyl]-N-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]piperidine-4-carboxamide (120 mg, 0.15 mmol), 2-chloro-5-(trifluoromethyl)pyrimidine (41 mg, 0.22 mmol), K₂CO₃ (41 mg, 0.30 mmol) in 1 mL DMF was stirred 12 h at 55° C. The mixture was quenched by water, extracted by EtOAc. The organic layer was washed with brine, dried over Na₂SO₄, filtered, and concentrated. The residue was purified by Prep-HPLC to afford the title compound as yellow solid. LCMS (ESI) m/z. [M+H]+=954.3.

The following compound was prepared by proceeding analogy as described in Example 44.

| Ex# | Structure | Synthesis |
|---|---|---|
| 45 | 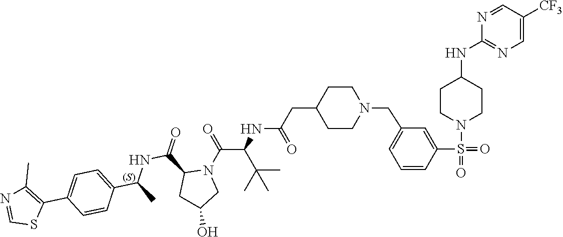 | 2-(1-tert-butoxycarbonyl- 4-piperidyl)-acetic acid replaced 1-tert-butoxy- carbonylpiperidine-4- carboxylic acid in Step 1 |

### Example 46

### Synthesis of 1-(6-(1-(1-(3-((4-((5-chloropyrimidin-2-yl)amino)piperidin-1-yl)sulfonyl)phenyl)-2,2-difluoroethyl)piperidin-4-yl)-1-methyl-1H-indazol-3-yl)dihydro pyrimidine-2,4(1H,3H)-dione

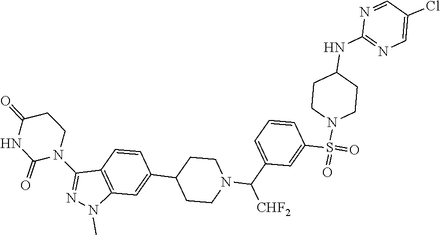 [SMILES: Cn1nc(N2CCC(=O)NC2=O)c2ccc(C3CCN(C(c4cccc(S(=O)(=O)N5CCC(Nc6ncc(Cl)cn6)CC5)c4)C(F)F)CC3)cc21]

### Step 1: Methyl 3-((4-((tert-butoxycarbonyl)amino)piperidin-1-yl)sulfonyl)benzoate

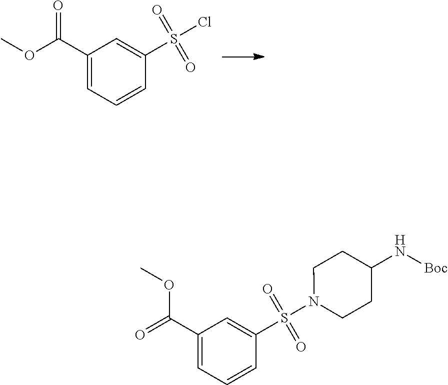 [SMILES: COC(=O)c1cccc(S(=O)(=O)Cl)c1 ; COC(=O)c1cccc(S(=O)(=O)N2CCC(NC(=O)OC(C)(C)C)CC2)c1]

To a mixture of tert-butyl N-(4-piperidyl)carbamate (4.27 g, 21.31 mmol, 1.0 eq) and TEA (3.56 mL, 25.57 mmol, 1.2 eq) in anhydrous DCM (30 mL) at 0° C. was added methyl 3-chlorosulfonylbenzoate (5 g, 21.31 mmol, 1.0 eq) in portions, and the mixture was stirred 3 h at 0° C. The mixture was diluted with water, extracted with DCM. The combined organic layers were washed with brine, and dried over anhydrous Na₂SO₄. After filtration, the filtrate was concentrated under reduced pressure. The residue was purified by silica gel column chromatography, eluted with EA/PE (0-40%) to afford the title compound as a white solid.

### Step 2: tert-Butyl (1-((3-(hydroxymethyl)phenyl)sulfonyl)piperidin-4-yl)carbamate

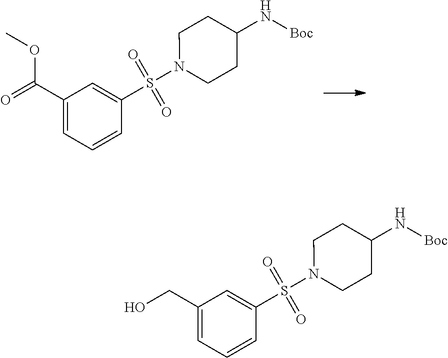 [SMILES: COC(=O)c1cccc(S(=O)(=O)N2CCC(NC(=O)OC(C)(C)C)CC2)c1 ; CC(C)(C)OC(=O)NC1CCN(S(=O)(=O)c2cccc(CO)c2)CC1]

To a stirred mixture of methyl 3-((4-((tert-butoxycarbonyl)amino)piperidin-1-yl)sulfonyl)benzoate (4. g, 10.04 mmol, 1.0 eq) in anhydrous THF (50 mL) at −78° C. was added DIBAL-H (35.14 mL, 35.14 mmol, 3.5 eq) dropwise. The resulting mixture was allowed to warm to 25° C. and stirred for 16 h. The mixture was quenched with water carefully at 0° C., followed by addition of 15% NaOH and anhydrous Na₂SO₄. The mixture was stirred at rt for 10 min, and filtered. After filtration, the filtrate was concentrated under reduced pressure. The residue was purified by silica gel column chromatography, eluted with EA/PE (0-40%) to afford title compound as a white solid.

### Step 3: tert-Butyl (1-((3-formylphenyl)sulfonyl)piperidin-4-yl)carbamate

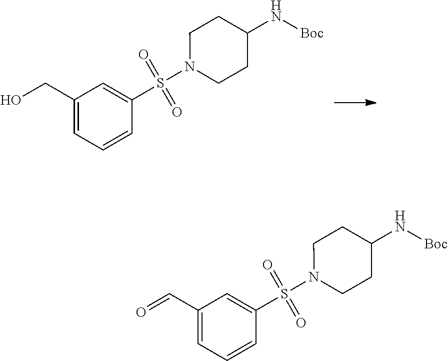 [SMILES: CC(C)(C)OC(=O)NC1CCN(S(=O)(=O)c2cccc(CO)c2)CC1 ; CC(C)(C)OC(=O)NC1CCN(S(=O)(=O)c2cccc(C=O)c2)CC1]

To a stirred mixture of tert-butyl (1-((3-(hydroxymethyl)phenyl)sulfonyl)piperidin-4-yl)carbamate (1.6 g, 4.32 mmol, 1.0 eq) in anhydrous DCM (30 mL) at 0° C. was added DMP (3.66 g, 8.64 mmol, 2.0 eq) in portions and the mixture was stirred for 2 h at 25° C. The mixture was diluted with water, quenched with saturated NaHCO₃ and saturated Na₂S₂O₃ at 0° C. and stirred at rt for 10 min. The mixture was extracted with DCM, and the combined organic layers were washed with brine, dried over anhydrous Na₂SO₄ and concentrated. The residue was purified by silica gel column chromatography, eluted with EA/PE (0-40%) to afford title compound as a white solid.

### Step 4: tert-Butyl (1-((3-(2,2-difluoro-1-hydroxyethyl)phenyl)sulfonyl)piperidin-4-yl)carbamate

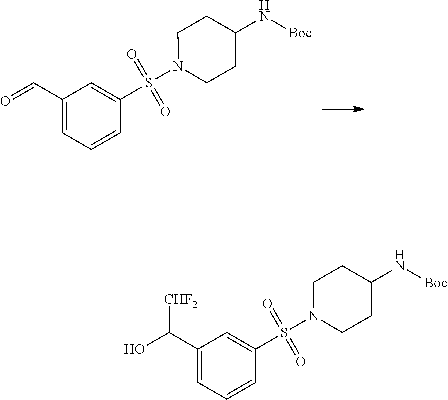 [SMILES: CC(C)(C)OC(=O)NC1CCN(S(=O)(=O)c2cccc(C=O)c2)CC1 ; CC(C)(C)OC(=O)NC1CCN(S(=O)(=O)c2cccc(C(O)C(F)F)c2)CC1]

A solution of difluoromethyl(trimethyl) silane (370.8 mg, 2.99 mmol, 2.0 eq) in DMF was added to a mixture of tert-butyl (1-((3-formylphenyl)sulfonyl)piperidin-4-yl)carbamate (550 mg, 1.49 mmol, 1.0 eq), CsF (226.8 mg, 1.49 mmol, 1.0 eq) in anhydrous DMF (7.5 mL) at 25° C., and the mixture was allowed to stir for 16 h. The mixture was quenched with ice/water and the mixture was extracted with EA. The combined organic layers were washed with brine, dried over anhydrous Na₂SO₄, and concentrated under reduced pressure. The residue was purified by silica gel column chromatography, eluted with EA/PE (0-45%) to afford the title compound as a white solid.

### Step 5: 1-(3-((4-((tert-Butoxycarbonyl)amino)piperidin-1-yl)sulfonyl)phenyl)-2,2-difluoroethyl methanesulfonate

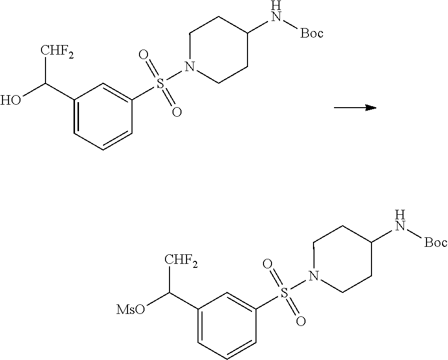 [SMILES: CC(C)(C)OC(=O)NC1CCN(S(=O)(=O)c2cccc(C(O)C(F)F)c2)CC1 ; CC(C)(C)OC(=O)NC1CCN(S(=O)(=O)c2cccc(C(OS(C)(=O)=O)C(F)F)c2)CC1]

MsCl (0.04 mL, 0.48 mmol, 2.0 eq) was added to a mixture of tert-butyl (1-((3-(2,2-difluoro-1-hydroxyethyl)phenyl)sulfonyl)piperidin-4-yl)carbamate (100 mg, 0.24 mmol, 1.0 eq) and DIPEA (0.12 mL, 0.72 mmol, 3.0 eq) in anhydrous DCM (5 mL) at 0° C., dropwise, and the mixture was stirred for 3 h at 25° C. The mixture was diluted with water, extracted with DCM. The combined organic layers were washed with brine, dried over anhydrous Na₂SO₄ and concentrated under reduced pressure. The residue was purified by silica gel column chromatography, eluted with EA/PE (0-20%) to afford the title compound as a yellow oil.

### Step 6: tert-Butyl (1-((3-(1-(4-(3-(2,4-dioxotetrahydropyrimidin-1(2H)-yl)-1-methyl-1H-indazol-6-yl)piperidin-1-yl)-2,2-difluoroethyl)phenyl)sulfonyl)piperidin-4-yl)carbamate

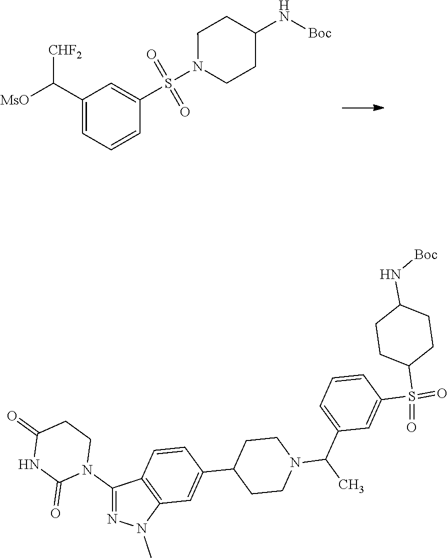 [SMILES: CC(C)(C)OC(=O)NC1CCN(S(=O)(=O)c2cccc(C(OS(C)(=O)=O)C(F)F)c2)CC1 ; CC(c1cccc(S(=O)(=O)C2CCC(NC(=O)OC(C)(C)C)CC2)c1)N1CCC(c2ccc3c(N4CCC(=O)NC4=O)nn(C)c3c2)CC1]

To a mixture of 1-[1-methyl-6-(4-piperidyl) indazol-3-yl]hexahydropyrimidine-2,4-dione (13.1 mg, 0.04 mmol, 1.0 eq) and 1-(3-((4-((tert-butoxycarbonyl)amino)piperidin-1-yl)-sulfonyl)phenyl)-2,2-difluoroethyl methanesulfonate (20 mg, 0.04 mmol, 1.0 eq) in anhydrous MeCN (3 mL) at 25° C. was added DIPEA (0.07 mL, 0.4 mmol, 10.0 eq). The resulting mixture was stirred for 3 days at 130° C. under Ar atmosphere and then concentrated under reduced pressure. The residue was diluted with water, extracted with DCM, washed with brine, dried over anhydrous Na₂SO₄, and concentrated under reduced pressure. The residue was purified by C18 column chromatography, eluted with MeCN/water (0-35%, 0.1% FA water) to afford the title compound as a yellow oil.

### Step 7: 1-(6-(1-(1-(3-((4-Aminopiperidin-1-yl)sulfonyl)phenyl)-2,2-difluoroethyl)piperidin-4-yl)-1-methyl-1H-indazol-3-yl)dihydropyrimidine-2,4(1H,3H)-dione 2,2,2-trifluoroacetate

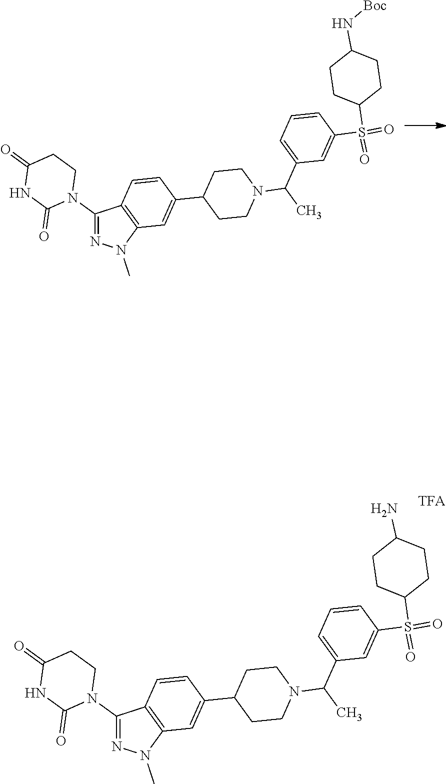 [SMILES: CC(c1cccc(S(=O)(=O)C2CCC(NC(=O)OC(C)(C)C)CC2)c1)N1CCC(c2ccc3c(N4CCC(=O)NC4=O)nn(C)c3c2)CC1 ; CC(c1cccc(S(=O)(=O)C2CCC(N)CC2)c1)N1CCC(c2ccc3c(N4CCC(=O)NC4=O)nn(C)c3c2)CC1]

To a stirred mixture of tert-butyl (1-((3-(1-(4-(3-(2,4-dioxotetrahydropyrimidin-1(2H)-yl)-1-methyl-1H-indazol-6-yl)piperidin-1-yl)-2,2-difluoroethyl)phenyl)sulfonyl)piperidin-4-yl) carbamate (10 mg, 0.0137 mmol, 1.0 eq) in anhydrous DCM (2 mL) at 25° C. was added TFA (0.02 mL), and the mixture was stirred for 2 h. The mixture was concentrated to afford the title compound as a yellow oil.

### Step 8: 1-[6-[1-[1-[3-[[4-[(5-Chloropyrimidin-2-yl)amino]-1-piperidyl]sulfonyl]phenyl]-2,2-difluoro-ethyl]-4-piperidyl]-1-methyl-indazol-3-yl]hexahydropyrimidine-2,4-dione

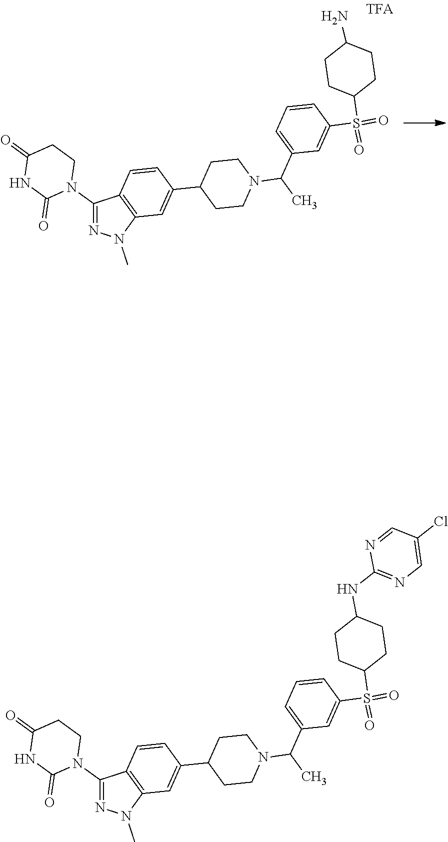 [SMILES: CC(c1cccc(S(=O)(=O)C2CCC(N)CC2)c1)N1CCC(c2ccc3c(N4CCC(=O)NC4=O)nn(C)c3c2)CC1 ; CC(c1cccc(S(=O)(=O)C2CCC(Nc3ncc(Cl)cn3)CC2)c1)N1CCC(c2ccc3c(N4CCC(=O)NC4=O)nn(C)c3c2)CC1]

A mixture of 1-(6-(1-(1-(3-((4-aminopiperidin-1-yl)sulfonyl)phenyl)-2,2-difluoroethyl)-piperidin-4-yl)-1-methyl-1H-indazol-3-yl)dihydropyrimidine-2,4(1H,3H)-dione 2,2,2-trifluoroacetate (0.0137 mmol, 1.0 eq), 2,5-dichloropyrimidine (6 mg, 0.04 mmol, 3.0 eq), and DIPEA (0.01 mL, 0.07 mmol, 5.0 eq) in anhydrous DMSO (2.5 mL) at 25° C. was stirred at 70° C. for 16 h. The mixture was purified by C18 column chromatography, eluted with MeCN/water (0-32%, 0.5% FA water) to afford the title compound as a white solid. LCMS (ESI) m/z [M+H]⁺=742.1.

### BIOLOGICAL EXAMPLES

### Biological Example 1

### Inhibition of CDK2: Phospho-R^{b} Measurement in Cells

Phosphorylation of RB protein at S807/811 were measured using HTRF phospho-RB cellular kits (Cat #64RBS807PEG) from Cisbio.

On Day 1, OVCAR3 (CDK2 dependent cell line) cells were seeded into 96-well tissue-culture treated plates at 20,000 cells/well in 200 μL and incubated overnight at 37° C. in CO₂ atmosphere. On Day 2, the cells were treated with test compounds at concentrations from 0.3 to 10,000 nM using HP D300 digital dispenser. Twenty-four hours after compound treatment, cell culture media is removed by flicking the plate and tapping the plate against clean paper towel. 30 μL 1× lysis buffer was supplemented from the kit was added to each well and the plate was incubated at room temperature on shaker for 30 min. After homogenization by pipetting up and down, 8 μL cell lysate from 96-well cell culture plate was transferred to 384-well small volume white detection plate. 2 μL premixed detection solution was added and the plate was covered with sealer. To prepare the detection solution, d2 conjugated-phospho-RB antibody and Eu-cryptate conjugated phosphor-RB antibody were diluted into detection buffer following manufacturer's instruction. Detection plates were incubated for 4 h at room temperature and read on ClarioStar (BMG Labtech) in TR-FRET mode (665 nM and 620 nM). The TR-FRET ratio (665 nM/620 nM) was plotted against the compound concentration and normalized to DMSO controls. Half maximal inhibition concentration (IC₅₀) values were calculated with a four-parameter logistic fit using GraphPad Prism (version 8; La Jolla, CA).

In the table below, A indicates a IC₅₀ of greater than or equal to 1 nM but less than or equal to 100 nM; B indicates a IC₅₀ of greater than 100 nM but less than or equal to 500 nM; C indicates a IC₅₀ of greater than 500 nM but less than or equal to 2.5 μM; D indicates a IC₅₀ of greater than 2.5 μM.

| | pRB_ | | pRB_ |
|---|---|---|---|
| | OVCAR3_ | | OVCAR3_ |
| Cpd No. | 24h | Cpd No. | 24h |
| (Cpd. Table 1) | (nM) | (Cpd Table 1) | (nM) |
| 1 | A | 36 | C |
| 2 | A | 38 | B |
| 3 | B | 39 | A |
| 4 | B | 40 | A |
| 5 | B | 41 | B |
| 6 | A | 42 | A |
| 7 | B | 43 | B |
| 8 | B | 45 | B |
| 9 | B | 46 | C |
| 10 | C | 47 | A |
| 11 | B | 48 | B |
| 12 | B | 49 | C |
| 13 | B | 50 | C |
| 14 | A | 51 | C |
| 15 | C | 52 | C |
| 16 | A | 53 | A |
| 17 | D | 54 | A |
| 18 | C | 55 | A |
| 19 | D | 56 | C |
| 20 | C | 57 | C |
| 21 | C | 58 | B |
| 22 | C | 59 | D |
| 23 | B | 60 | C |
| 24 | B | 61 | A |
| 25 | B | 62 | B |
| 26 | D | 63 | A |
| 27 | B | 64 | A |
| 28 | B | 65 | C |
| 29 | A | 66 | A |
| 30 | C | 67 | D |
| 32 | C | | |
| 33 | B | | |
| 34 | B | | |
| 35 | A | | |
| 70 | D | 109 | B |
| 71 | A | 110 | B |
| 72 | C | 111 | C |
| 73 | C | 112 | A |
| 74 | B | 113 | B |
| 75 | B | 114 | B |
| 76 | A | 115 | A |
| 77 | A | 116 | A |
| 78 | B | 117 | B |
| 79 | C | 118 | A |
| 80 | B | 119 | A |
| 81 | C | 120 | B |
| 82 | C | 121 | A |
| 83 | A | 122 | A |
| 84 | D | 123 | A |
| 85 | C | 124 | A |
| 86 | A | 125 | C |
| 87 | A | 126 | C |
| 88 | C | 127 | A |
| 89 | A | 128 | A |
| 90 | B | 129 | A |
| 91 | B | 130 | A |
| 92 | B | 131 | A |
| 93 | A | 132 | A |
| 94 | A | 133 | B |
| 95 | A | 134 | B |
| 96 | B | 135 | B |
| 97 | C | 136 | B |
| 98 | B | 137 | B |
| 99 | A | 138 | D |
| 100 | A | 139 | D |
| 101 | B | 140 | A |
| 102 | C | 141 | C |
| 103 | C | 142 | A |
| 104 | D | 143 | C |
| 105 | B | 144 | B |
| 106 | B | 145 | B |
| 107 | A | 146 | D |
| 108 | A | | |
| 147 | A | 199 | A |
| 148 | A | 200 | B |
| 149 | A | 201 | A |
| 150 | A | 202 | A |
| 151 | A | 203 | A |
| 152 | A | 204 | A |
| 153 | A | 205 | A |
| 154 | D | 206 | A |
| 155 | D | 207 | B |
| 156 | B | 208 | A |
| 157 | C | 209 | A |
| 158 | C | 210 | B |
| 159 | B | 211 | A |
| 160 | B | 212 | C |
| 161 | D | 213 | B |
| 162 | A | 214 | A |
| 163 | D | 215 | C |
| 164 | D | 216 | A |
| 165 | A | 217 | A |
| 166 | D | 218 | D |
| 167 | C | 219 | D |
| 168 | A | 220 | A |
| 169 | B | 221 | A |
| 170 | C | 222 | A |
| 171 | B | 223 | A |
| 172 | D | 224 | A |
| 173 | C | 225 | B |
| 174 | A | 226 | B |
| 175 | B | 227 | C |
| 176 | C | 228 | B |
| 177 | A | 229 | D |
| 178 | D | 230 | C |
| 179 | C | 231 | C |
| 180 | B | 232 | B |
| 181 | A | 233 | B |
| 182 | B | 234 | A |
| 183 | C | 235 | B |
| 184 | A | 236 | B |
| 185 | C | 237 | C |
| 186 | A | 238 | A |
| 187 | B | 239 | B |
| 188 | A | 240 | D |
| 189 | D | 241 | A |
| 190 | A | 242 | C |
| 191 | D | 243 | C |
| 192 | A | 244 | A |
| 193 | C | 245 | D |
| 194 | B | 246 | C |
| 195 | C | 247 | A |
| 196 | B | 248 | A |
| 197 | A | 249 | B |
| 198 | A | 250 | A |
| 251 | A | 252 | B |
| 253 | D | 254 | B |
| 255 | B | 256 | B |
| 257 | B | 258 | B |
| 259 | C | 260 | B |
| 261 | B | | |

### Biological Example 2

### High-Throughput Measurement of Cellular Endogenous CDK2

Effects of compounds on cellular CDK2 level were monitored by a high-throughput HTRF assay.

To determine half maximal degradation concentration (DC₅₀) and maximum degradation level (Dmax) values of compounds, cellular CDK2 level was measured in 96-well format using HTRF total CDK2 cellular kit (Cat #64CDK2TPEG) from Cisbio.

On Day 1, OVCAR3 cells were seeded into 96-well tissue-culture treated plates at 20,000 cells/well in 200 μL and incubated overnight at 37° C. in CO₂ atmosphere. On Day 2 cells were treated with compounds at concentration ranging from 0.03 to 1,000 nM using HP D300 digital dispenser. 6 hours after compound treatment, cell culture media was removed by flicking the plate and tapping the plate against clean paper towel. Immediately 30 μL 1× lysis buffer was supplemented from the kit and added to each well and the plate is incubated at room temperature on shaker for 30 min. After homogenization by pipetting up and down, 8 μL cell lysate from 96-well cell culture plate was transferred to 384-well small volume white detection plate. 2 μL premixed detection solution was added and the plate is covered with sealer. To prepare the detection solution, d2 conjugated-CDK2 antibody and Eu-cryptate conjugated CDK2 antibody were diluted into detection buffer following manufacturer's instruction. Detection plates were incubated overnight at room temperature and read on ClarioStar (BMG Labtech) in TR-FRET mode (665 nM and 620 nM). The TR-FRET ratio (665 nM/620 nM) was plotted against the compound concentration and normalized to DMSO controls (0% degradation) and lysis buffer controls (100% degradation). Half maximal degradation concentration (DC 50) (0% degradation) and lysis buffer controls (100% degradation) values were calculated with a four-parameter logistic fit using GraphPad Prism (version 8; La Jolla, CA).

In the table below, AA indicates a DC₅₀ of greater than or equal to 0.5 nM but less than 1 nM; A indicates a DC₅₀ of greater than or equal to 1 nM but less than or equal to 10 nM; B indicates a DC₅₀ of greater than 10 nM but less than or equal to 100 nM; C indicates a DC₅₀ of greater than 100 nM but less than or equal to 1 μM; and D indicates a DC₅₀ of greater than 1 μM nM but less than or equal to 5 μM. NT means not tested.

TABLE 1

| Cpd No. | DC50_ | Cpd No. | DC50_ |
|---|---|---|---|
| (Cpd. | OVCAR3_ | (Cpd. | OVCAR3_ |
| Table 1) | 6h | Table 1) | 6h |
| 1 | A | 70 | NT |
| 2 | B | 71 | A |
| 3 | NT | 72 | NT |
| 4 | NT | 73 | NT |
| 5 | NT | 74 | A |
| 6 | C | 75 | NT |
| 7 | C | 76 | A |
| 8 | A | 77 | A |
| 9 | NT | 78 | NT |
| 10 | NT | 79 | NT |
| 11 | A | 80 | NT |
| 12 | NT | 81 | NT |
| 13 | NT | 82 | NT |
| 14 | B | 83 | A |
| 15 | NT | 84 | NT |
| 16 | B | 85 | NT |
| 17 | NT | 86 | B |
| 18 | NT | 87 | A |
| 19 | NT | 88 | NT |
| 20 | NT | 89 | B |
| 21 | NT | 90 | NT |
| 22 | NT | 91 | B |
| 23 | B | 92 | A |
| 24 | A | 93 | A |
| 25 | NT | 94 | B |
| 26 | NT | 95 | A |
| 27 | B | 96 | NT |
| 28 | B | 97 | NT |
| 29 | A | 98 | NT |
| 30 | NT | 99 | B |
| 31 | NT | 100 | B |
| 32 | NT | 101 | NT |
| 33 | B | 102 | NT |
| 34 | NT | 103 | NT |
| 35 | B | 104 | NT |
| 36 | NT | 105 | B |
| 37 | NT | 106 | NT |
| 38 | B | 107 | A |
| 39 | A | 108 | B |
| 40 | A | 109 | NT |
| 41 | NT | 110 | NT |
| 42 | A | 111 | NT |
| 43 | B | 112 | A |
| 44 | NT | 113 | NT |
| 45 | NT | 114 | NT |
| 46 | NT | 115 | B |
| 47 | B | 116 | B |
| 48 | NT | 117 | NT |
| 49 | NT | 118 | NT |
| 50 | NT | 119 | A |
| 51 | NT | 120 | NT |
| 52 | NT | 121 | NT |
| 53 | B | 122 | NT |
| 54 | A | 123 | NT |
| 55 | B | 124 | NT |
| 56 | NT | 125 | NT |
| 57 | NT | 126 | NT |
| 58 | A | 127 | NT |
| 59 | NT | 128 | NT |
| 60 | NT | 129 | NT |
| 61 | B | 130 | NT |
| 62 | NT | 131 | NT |
| 63 | NT | 132 | NT |
| 64 | A | 133 | NT |
| 65 | NT | 134 | NT |
| 66 | A | 135 | NT |
| 67 | NT | | |
| 69 | NT | | |
| 147 | A | 199 | A |
| 148 | B | 200 | NT |
| 149 | A | 201 | A |
| 150 | B | 202 | A |
| 151 | B | 203 | A |
| 152 | A | 204 | A |
| 153 | B | 205 | AA |
| 154 | NT | 206 | B |
| 155 | NT | 207 | NT |
| 156 | NT | 208 | A |
| 157 | NT | 209 | B |
| 158 | NT | 210 | NT |
| 159 | NT | 211 | D |
| 160 | NT | 212 | NT |
| 161 | NT | 213 | NT |
| 162 | A | 214 | B |
| 163 | NT | 215 | NT |
| 164 | NT | 216 | B |
| 165 | B | 217 | B |
| 166 | NT | 218 | NT |
| 167 | NT | 219 | NT |
| 168 | B | 220 | B |
| 169 | NT | 221 | A |
| 170 | NT | 222 | A |
| 171 | NT | 223 | A |
| 172 | NT | 224 | B |
| 173 | NT | 225 | NT |
| 174 | A | 226 | B |
| 175 | B | 227 | NT |
| 176 | NT | 228 | NT |
| 177 | A | 229 | NT |
| 178 | NT | 230 | NT |
| 179 | NT | 231 | NT |
| 180 | B | 232 | NT |
| 181 | A | 233 | NT |
| 182 | NT | 234 | A |
| 183 | NT | 235 | NT |
| 184 | A | 236 | NT |
| 185 | NT | 237 | NT |
| 186 | NT | 238 | B |
| 187 | NT | 239 | B |
| 188 | B | 240 | NT |
| 189 | NT | 241 | A |
| 190 | B | 242 | NT |
| 191 | NT | 243 | NT |
| 192 | A | 244 | A |
| 193 | NT | 245 | NT |
| 194 | NT | 246 | NT |
| 195 | NT | 247 | B |
| 196 | NT | 248 | D |
| 197 | B | 249 | NT |
| 198 | A | 250 | B |
| 251 | B | 252 | NT |
| 253 | NT | 254 | NT |
| 255 | NT | 256 | NT |
| 257 | NT | 258 | NT |
| 259 | NT | 260 | NT |
| 261 | NT | | |

### Formulation Examples

The following are representative pharmaceutical formulations containing a compound of the present disclosure.

Tablet Formulation
The following ingredients are mixed intimately and
pressed into single scored tablets.

| Ingredient | Quantity per tablet (mg) |
|---|---|
| compound Formula | 400 |
| (A1), (A), (I), or (Ia) | |
| cornstarch | 50 |
| croscarmellose sodium | 25 |
| lactose | 120 |
| magnesium stearate | 5 |

Capsule Formulation
The following ingredients are mixed intimately and
loaded into a hard-shell gelatin capsule.

| Ingredient | Quantity per capsule (mg) |
|---|---|
| Compound Formula | 200 |
| (A1), (A), (I), or (Ia) | |
| lactose spray dried | 148 |
| magnesium stearate | 2 |

### Injectable Formulation

Compound of the disclosure in 2% HPMC, 1% Tween 80 in DI water, pH 2.2 with MSA, q.s. to at least 20 mg/mL

### Inhalation Composition

To prepare a pharmaceutical composition for inhalation delivery, 20 mg of a compound disclosed herein is mixed with 50 mg of anhydrous citric acid and 100 mL of 0.9% sodium chloride solution. The mixture is incorporated into an inhalation delivery unit, such as a nebulizer, which is suitable for inhalation administration.

### Topical Gel Composition

To prepare a pharmaceutical topical gel composition, 100 mg of a compound disclosed herein is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Ophthalmic Solution Composition

To prepare a pharmaceutical ophthalmic solution composition, 100 mg of a compound disclosed herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Nasal Spray Solution

To prepare a pharmaceutical nasal spray solution, 10 g of a compound disclosed herein is mixed with 30 mL of a 0.05M phosphate buffer solution (pH 4.4). The solution is placed in a nasal administrator designed to deliver 100 μL of spray for each application.

What is claimed:

1. A compound of Formula (Ia): 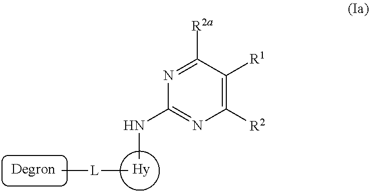
wherein:
R¹ is alkyl, alkenyl, alkynyl, cycloalkyl, halo, haloalkyl, haloalkoxy, alkoxy, aryloxy, cyano, cycloalkyl where the cycloalkyl is substituted with one to three halo;
R² and R^{2a} are independently hydrogen or deuterium;
Hy is cycloalkylene, arylene, heteroarylene, heterocyclylene, bicyclic heterocyclylene, spiro heterocyclylene, bridged heterocyclylene, or fused heterocyclylene, where each of the aforementioned rings is substituted with R^{a}, R^{b}, and R^{c} independently selected from hydrogen, deuterium, alkyl, halo, haloalkyl, alkoxy, hydroxy, and cyano;
Degron is an E3 ubiquitin ligase ligand selected from:
(a) a group of formula (i): 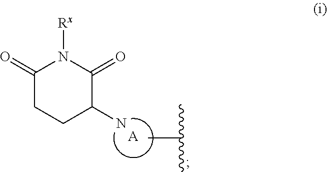
(b) a group of formula (ii): 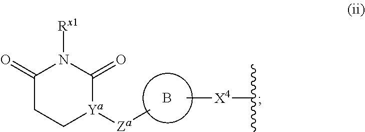
(c) a group of formula (iii): 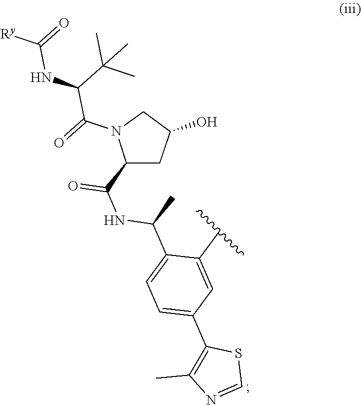
(d) a group of formula (iv): 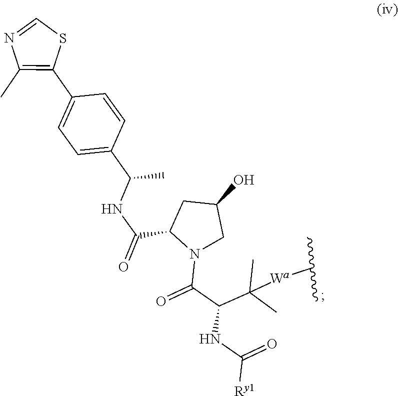
(e) a group of formula (v): 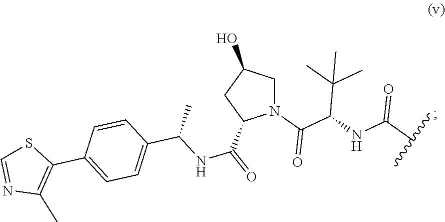 [SMILES: *C(=O)N[C@H](C(=O)N1C[C@H](O)C[C@H]1C(=O)N[C@@H](C)c1ccc(-c2scnc2C)cc1)C(C)(C)C]
and
(f) a group of formula (vi): 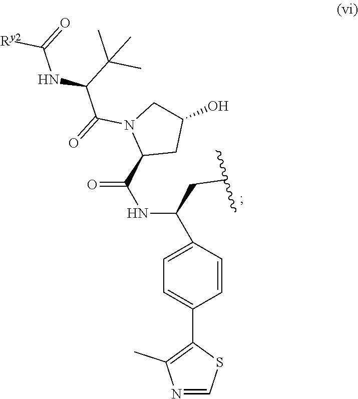
where:
R^{x} and R^{x1} are each hydrogen;
Y^{a} is CH or N;
Z^{a} is a bond, —CH₂—, —NH—, —O—, or —NHC(O)— where NH of —NHC(O)— is attached to Y^{a}:
ring A is a group of formula (a), (b), or (c): 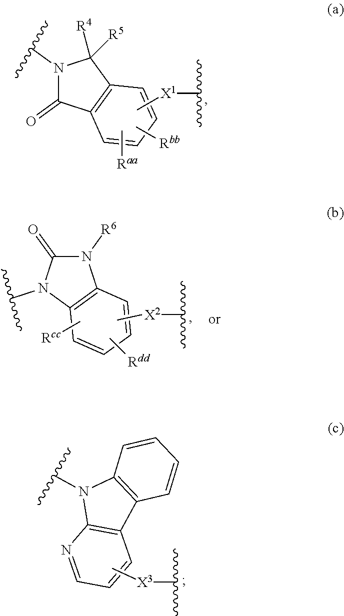
where:
R^{aa}, R^{bb}, R^{cc}, and R^{dd} are independently selected from hydrogen, alkyl, alkoxy, halo, haloalkyl, haloalkoxy, and cyano;
R⁴ and R⁵ are independently hydrogen or alkyl; or R⁴ and R⁵ together with the carbon to which they are attached form >C═O; and
R⁶ is hydrogen or alkyl;
ring B is phenylene, cyclylaminylene, a 5- or 6-membered monocyclic heteroarylene, or a 9- or 10-membered fused bicyclic heteroarylene, wherein each heteroarylene ring contains one to three nitrogen ring atoms and further wherein the phenylene, cyclylaminylene, and each heteroarylene are independently substituted with R^{ee} and R^{ff} independently selected from hydrogen, alkyl, cycloalkyl, alkoxy, halo, haloalkyl, haloalkoxy, and cyano; and
X¹, X², X³, and X⁴ are independently a bond, -alkylene-, —O—, —(O-alkylene)-, (alkylene-O)—, —(NR^{gg}-alkylene)-, -(alkylene-NR^{hh})—, 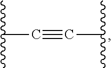 [SMILES: *C#C*]
—NH—, —N(alkyl)-, —C(═O)—, —NR^{jj}C(═O)—, or —C(═O)NR^{kk}— where R^{gg}, R^{hh}, R^{jj}, and R^{kk} are independently hydrogen, alkyl, or cycloalkyl and each alkylene, itself or as part of another group, is optionally substituted with one or two fluoro;
R^{y}, R^{y1}, and R^{y2} are independently alkyl, hydroxyalkyl, cycloalkyl or heterocyclyl wherein cycloalkyl and heterocyclyl are substituted with R^{d} and R^{f} selected from hydrogen, halo, cyano, alkylcarbonyl, and alkylcarbonylamino; and
W^{a} is bond, O, S, or alkylene; and
L is —Z¹—Z²—Z³—Z⁴—Z⁵—Z⁶— where:
Z¹ is a bond, alkylene, —C(O)NR—, —NR′(CO)—, —S(O)₂NR—, —NR′S(O)₂—, —(O-alkylene)ₐ-, -(alkylene-O)ₐ—, phenylene, monocyclic heteroarylene, or heterocyclylene, where each ring is substituted with R^{h} and Rⁱ independently selected from hydrogen, deuterium, alkyl, alkoxy, halo, haloalkyl, haloalkoxy, cyano, hydroxy, amino, alkylamino, and dialkylamino;
Z² is a bond, alkylene, alkynylene, —C(O)—, —C(O)N(R)—, —NR′(CO)—, —(O-alkylene)-, -(alkylene-O)_{b}—, —O(CH₂)₇—, —O(CH₂)₈—, cycloalkylene, or -heterocyclylene, where each ring is substituted with R^{j} and R^{k} independently selected from hydrogen, deuterium, alkyl, alkoxy, halo, haloalkyl, haloalkoxy, cyano, hydroxy, amino, alkylamino, and dialkylamino;
Z³ is a bond, alkylene, alkynylene, —C(O)NR—, —NR′(CO)—, —O—, —NR″-, —(O-alkylene)_{c}-, -(alkylene-O)_{c}—, cycloalkylene, spiro cyclolalkylene, phenylene, -(alkylene)-phenylene-, -phenylene-(alkylene)-, monocyclic heteroarylene, -(alkylene)-monocyclic heteroarylene-, -monocyclic heteroarylene-(alkylene)-, heterocyclylene, -(alkylene)-heterocyclylene-, -heterocyclylene-(alkylene)-, bicyclic heterocyclylene, bridged heterocyclylene, -(alkylene)-bridged heterocyclylene-, -bridged heterocyclylene-(alkylene)-, fused heterocyclylene, spiro heterocyclylene, -(alkylene)-spiro heterocyclylene-, -spiro heterocyclylene-(alkylene)-, or 11 to 13 membered spiro heterocyclylene, where each ring, by itself or as part of another group, is substituted with R^{m} and Rⁿ independently selected from hydrogen, deuterium, alkyl, alkoxy, halo, haloalkyl, haloalkoxy, cyano, hydroxy, amino, alkylamino, and dialkylamino;
Z⁴ is a bond, alkylene, alkynylene, -(alkylene-NR″)—, —(NR″-alkylene)-, —O—, —C(O)—, —NR″—, —(O-alkylene)_{d}-, -(alkylene-O)_{d}—, cycloalkylene, -(alkylene)-cycloalkylene-, -cycloalkylene-(alkylene)-, spiro cyclolalkylene, phenylene, heteroarylene, heterocyclylene, -(alkylene)-heterocyclylene-, -heterocyclylene-(alkylene)-, fused heterocyclylene, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p} independently selected from hydrogen, deuterium, alkyl, alkoxy, halo, haloalkyl, haloalkoxy, cyano, hydroxy, amino, alkylamino, and dialkylamino;
Z⁵ is a bond, -alkylene, —NR″—, —O—, —C(O)—, —S(O)₂—, —NR′(CO)—, —C(O)NR—, phenylene, monocyclic heteroarylene, or heterocycylene, where each ring is substituted with R^{q} and R^{r} independently selected from hydrogen, deuterium, alkyl, alkoxy, halo, haloalkyl, haloalkoxy, cyano, hydroxy, amino, alkylamino, and dialkylamino; and
Z⁶ is a bond, alkylene, —NR″—, —O—, -(alkylene-O)—, —C(O)—, —S(O)₂—, —NR′(CO)—, or —C(O)NR—;
where each R, R′ and R″ is independently hydrogen or alkyl, each a, b, c, and d is independently an integer selected from 1 to 6, and each alkylene of —Z¹—, —Z²—, —Z³—, —Z⁴—, —Z⁵— and —Z⁶—, by itself or as part of another group, is independently substituted with R^{s} and R^{t} where R^{s} is hydrogen or deuterium and R^{t} is hydrogen, deuterium, haloalkyl, hydroxy, alkoxy, cyano, cycloalkyl, heterocyclyl, aryl, or monocyclic heteroaryl, wherein cycloalkyl, heterocyclyl, aryl, monocyclic heteroaryl are substituted with one or two substituents independently selected from hydrogen, alkyl, alkoxy, halo, haloalkyl, haloalkoxy, or cyano; provided that at least one of —Z¹—Z²—Z³—Z⁴—Z⁵—Z⁶— is not a bond; or
a pharmaceutically acceptable salt thereof.

2. The compound of claim 1, or a pharmaceutically acceptable salt thereof, wherein R¹ is halo, haloalkyl, or haloalkoxy.

3. The compound of claim 1 or 2, or a pharmaceutically acceptable salt thereof, wherein R¹ is halo.

4. The compound of claim 1 or 2, or a pharmaceutically acceptable salt thereof, wherein R¹ is haloalkyl.

5. The compound of claim 1 or 2, or a pharmaceutically acceptable salt thereof, wherein R¹ is haloalkoxy.

6. The compound of claim 1, or a pharmaceutically acceptable salt thereof, wherein R¹ is chloro, bromo, fluoro, difluoromethyl, trifluoromethyl, difluoroethyl, trifluoroethyl, difluoromethoxy, trifluoromethoxy, difluoroethoxy, or trifluoroethoxy.

7. The compound of any one of claims 1 to 3 and 6, or a pharmaceutically acceptable salt thereof, wherein R¹ is chloro or bromo.

8. The compound of any one of claims 1, 2, 4, and 6, or a pharmaceutically acceptable salt thereof, wherein R¹ is difluoromethyl or trifluoromethyl.

9. The compound of any one of claims 1 to 8, or a pharmaceutically acceptable salt thereof, wherein both R² and R^{2a} are hydrogen.

10. The compound of any one of claims 1 to 8, or a pharmaceutically acceptable salt thereof, wherein one of R² and R^{2a} is deuterium and the other of R² and R^{2a} is hydrogen or both R² and R^{2a} are deuterium.

11. The compound of any one of claims 1 to 10, or a pharmaceutically acceptable salt thereof, wherein Hy is heterocyclylene, phenylene, spiro heterocyclylene, bridged heterocyclylene, or cycloalkylene, wherein each of the aforementioned rings is substituted with R^{a}, R^{b}, and R^{c} where R^{a} and R^{b} are independently selected from hydrogen, deuterium, alkyl, halo, haloalkyl, alkoxy, and hydroxy and R^{c} is hydrogen.

12. The compound of any one of claims 1 to 11, or a pharmaceutically acceptable salt thereof, wherein Hy is heterocyclylene substituted with R^{a}, R^{b}, and R^{c} where R^{a} and R^{b} are independently selected from hydrogen, deuterium, alkyl, halo, haloalkyl, alkoxy, and hydroxy and R^{c} is hydrogen.

13. The compound of any one of claims 1 to 12, or a pharmaceutically acceptable salt thereof, wherein the heterocyclylene of Hy is pyrrolidin-1,3-diyl or piperidin-1,4-diyl, where Hy is substituted with R^{a}, R^{b}, and R^{c} where R^{a} and R^{D} are independently hydrogen, deuterium, methyl, fluoro, methoxy, or hydroxy, R^{c} is hydrogen, and L is attached to the nitrogen atom of the piperidin-1,4-diyl or pyrrolidin-1,3-diyl ring of Hy.

14. The compound of any one of claims 1 to 13, or a pharmaceutically acceptable salt thereof, wherein the heterocyclylene of Hy is: 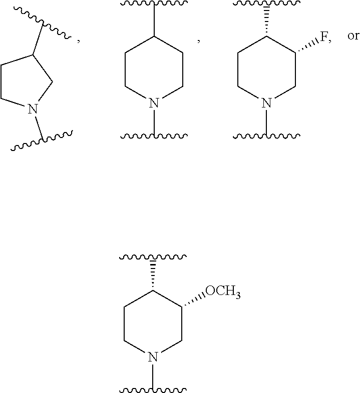 [SMILES: *C1CCN(*)C1 ; *C1CCN(*)CC1 ; *[C@H]1CCN(*)C[C@H]1F ; "or" ; *[C@H]1CCN(*)C[C@H]1OC]
where the N atom of the pyrrolidin-1,3-diyl or piperidin-1,4-diyl rings is attached to L.

15. The compound of any one of claims 1 to 14, or a pharmaceutically acceptable salt thereof, wherein the heterocyclylene of Hy is: 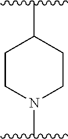 [SMILES: *C1CCN(*)CC1]
where the N atom of piperidin-1,4-diyl rings is attached to L.

16. The compound of any one of claims 1 to 15, or a pharmaceutically acceptable salt thereof, wherein the Degron is an E3 ubiquitin ligase ligand of formula (i):

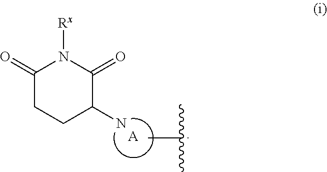

17. The compound of any one of claims 1 to 16, or a pharmaceutically acceptable salt thereof, wherein the ring A of the E3 ubiquitin ligase ligand of formula (i) is a group of formula (a):

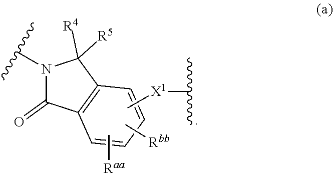

18. The compound of any one of claims 1 to 16, or a pharmaceutically acceptable salt thereof, wherein the ring A of the E3 ubiquitin ligase ligand of formula (i) is a group of formula (b):

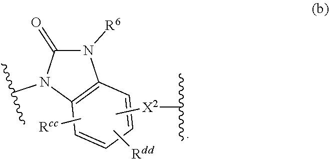

19. The compound of any one of claims 1 to 18, or a pharmaceutically acceptable salt thereof, wherein the ring A of the E3 ubiquitin ligase ligand of formula (i) is:

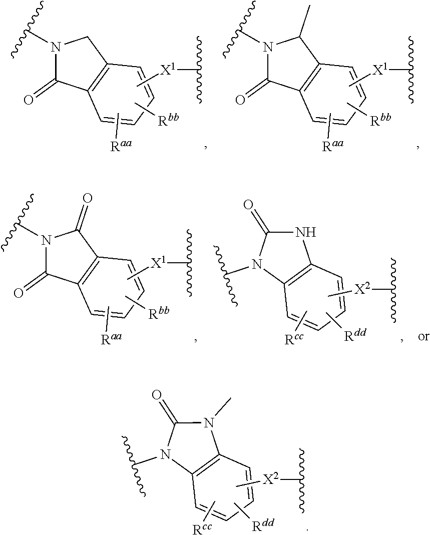

20. The compound of any one of claims 1 to 19, or a pharmaceutically acceptable salt thereof, wherein ring A of the E3 ubiquitin ligase ligand of formula (i) is:

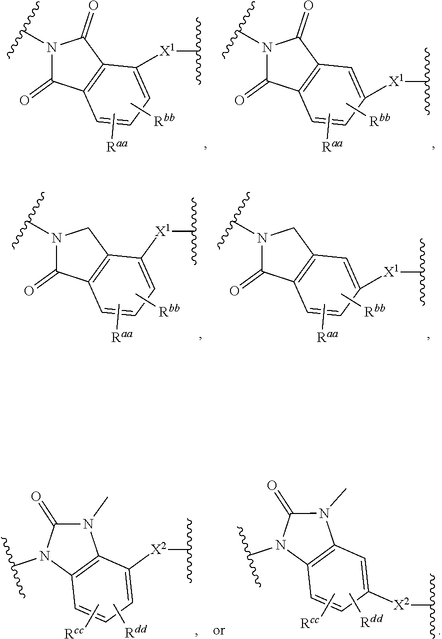

21. The compound of any one of claims 1 to 20, or a pharmaceutically acceptable salt thereof, wherein ring A of the E3 ubiquitin ligase ligand of formula (i) is:

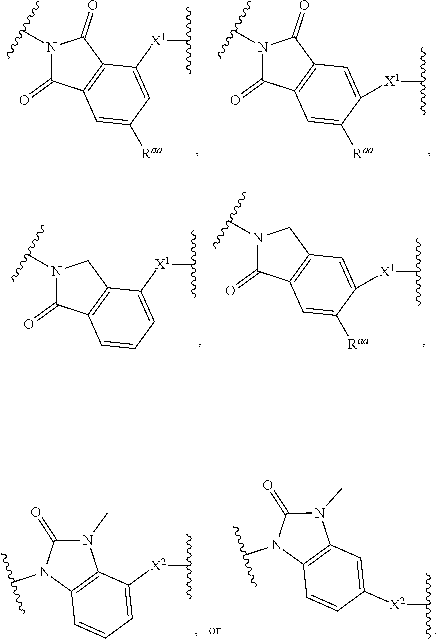

22. The compound of any one of claims 1 to 21, or a pharmaceutically acceptable salt thereof, wherein R^{aa}, R^{bb}, R^{cc}, and R^{dd} are independently selected from hydrogen, alkyl, alkoxy, halo, haloalkyl, and haloalkoxy.

23. The compound of any one of claims 1 to 22, or a pharmaceutically acceptable salt thereof, wherein R^{aa}, R^{bb}, R^{cc}, and R^{dd} are independently selected from hydrogen, methyl, methoxy, ethoxy, fluoro, trifluoromethyl, difluoromethyl, and trifluoromethoxy.

24. The compound of any one of claims 1 to 15, or a pharmaceutically acceptable salt thereof, wherein the Degron is an E3 ubiquitin ligase ligand of formula (ii):

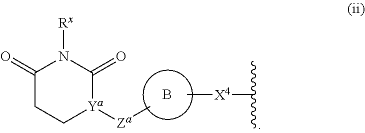

25. The compound of any one of claims 1 to 15 and 24, or a pharmaceutically acceptable salt thereof, wherein the E3 ubiquitin ligase ligand of formula (ii) is:

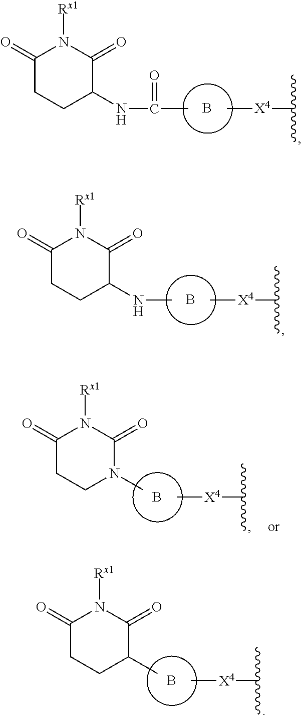

26. The compound of any one of claims 1 to 15, 24, and 25, or a pharmaceutically acceptable salt thereof, wherein the E3 ubiquitin ligase ligand of formula (ii) is: 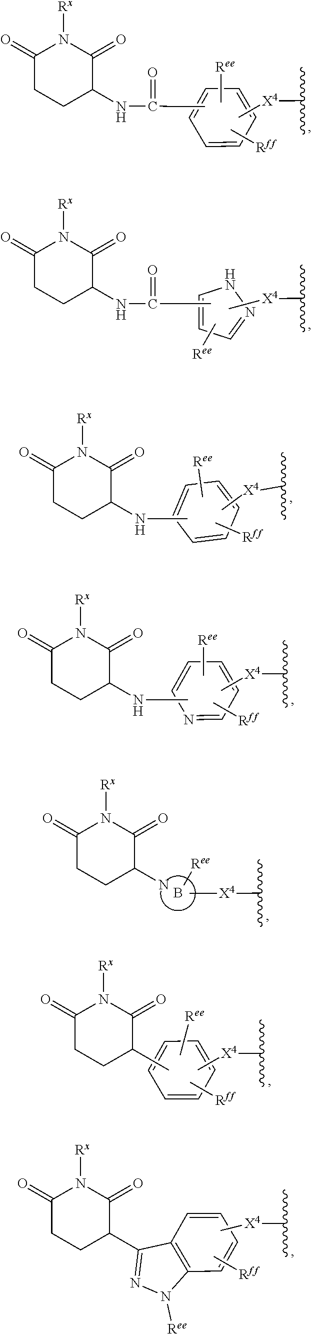 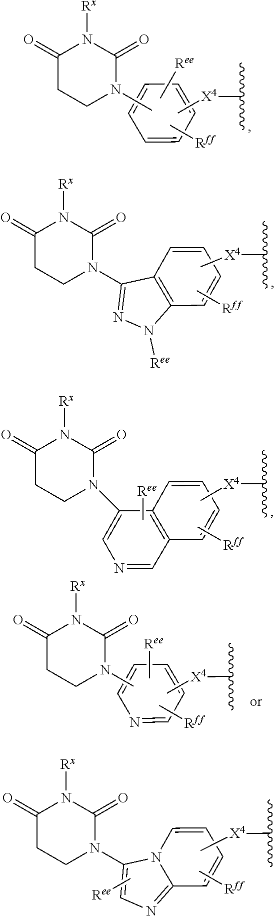
where ring B is cyclylaminylene.

27. The compound of any one of claims 1 to 26, or a pharmaceutically acceptable salt thereof, wherein X¹, X², X³, and X⁴ are each a bond.

28. The compound of any one of claims 1 to 27, or a pharmaceutically acceptable salt thereof, wherein Z⁶ is —S(O)₂—.

29. The compound of any one of claims 1 to 28, or a pharmaceutically acceptable salt thereof, wherein Z⁵ is phenylene, monocyclic heteroarylene, or heterocycylene, where each ring is substituted with R^{q} and R^{r}.

30. The compound of any one of claims 1 to 26, or a pharmaceutically acceptable salt thereof, wherein:
X¹, X², X³, X⁴, and Z¹ are each a bond;
Z² is a bond, alkylene, cycloalkylene, or heterocyclylene, where each ring is substituted with R^{j} and R^{k};
Z³ is a bond, alkylene, —C(O)NR—, —NR′(CO)—, —O—, —NR″—, cycloalkylene, phenylene, monocyclic heteroarylene, heterocyclylene, bicyclic heterocyclylene, bridged heterocyclylene, fused heterocyclylene, or spiro heterocyclylene, where each ring is substituted with R^{m} and Rⁿ;
Z⁴ is a bond, alkylene, —O—, cycloalkylene, phenylene, monocyclic heteroarylene, heterocyclylene, fused heterocyclylene, or spiro heterocyclylene, where each ring is substituted with R^{o} and R^{p};
Z⁵ is phenylene, monocyclic heteroarylene, or heterocycylene, where each ring is substituted with R^{q} and R^{r}; and
Z⁶ is —S(O)₂—; and
wherein each alkylene in Z², Z³, and Z⁴ is independently substituted with R^{s} and R^{t}.

31. The compound of any one of claims 1 to 26 and 30, or a pharmaceutically acceptable salt thereof, wherein:
X¹, X², X³, X⁴, Z¹, and Z² are each a bond;
Z³ is cycloalkylene, phenylene, monocyclic heteroarylene, heterocyclylene, bicyclic heterocyclylene, bridged heterocyclylene, fused heterocyclylene, or spiro heterocyclylene, where each ring is substituted with R^{m} and Rⁿ;
Z⁴ is a bond, alkylene, —O—, cycloalkylene, phenylene, monocyclic heteroarylene, heterocyclylene, fused heterocyclylene, or spiro heterocyclylene, where each ring is substituted with R^{o} and R^{p} independently selected from hydrogen, deuterium, alkyl, alkoxy, hydroxy, cyano, halo, haloalkyl, and haloalkoxy;
Z⁵ is phenylene, monocyclic heteroarylene, or heterocycylene, where each ring is substituted with R^{q} and R^{r}; and
Z⁶ is —S(O)₂—; and
wherein alkylene in Z⁴ is substituted with R^{s} and R^{t}.

32. The compound of any one of claims 1 to 26, 30, and 31, or a pharmaceutically acceptable salt thereof, wherein:
X¹, X², X³, X⁴, Z¹, and Z² are each a bond;
Z³ is heterocyclylene, bicyclic heterocyclylene, bridged heterocyclylene, fused heterocyclylene, or spiro heterocyclylene, where each ring is substituted with R^{m} and Rⁿ independently selected from hydrogen, deuterium, alkyl, alkoxy, hydroxy, cyano, halo, haloalkyl, and haloalkoxy;
Z⁴ is alkylene, —O—, cycloalkylene, monocyclic heteroarylene, heterocyclylene, fused heterocyclylene, or spiro heterocyclylene, where each ring is substituted with R^{o} and R^{p} independently selected from hydrogen, deuterium, alkyl, alkoxy, hydroxy, cyano, halo, haloalkyl, and haloalkoxy;
Z⁵ is phenylene, monocyclic heteroarylene, or heterocycylene, where each ring is substituted with R^{q} and R^{r} independently selected from hydrogen, deuterium, alkyl, alkoxy, hydroxy, cyano, halo, haloalkyl, and haloalkoxy; and
Z⁶ is —S(O)₂—; and
wherein alkylene in Z⁴ is substituted with R^{s} and R^{t}.

33. The compound of any one of claims 1 to 26 and 30 to 32, or a pharmaceutically acceptable salt thereof, wherein:
X¹, X², X³, and X⁴, Z¹, and Z² are each a bond;
Z³ is heterocyclylene, bridged heterocyclylene, or spiro heterocyclylene, where each ring is substituted with R^{m} and Rⁿ independently selected from hydrogen, deuterium, alkyl, alkoxy, hydroxy, cyano, halo, haloalkyl, and haloalkoxy;
Z⁴ is alkylene, —O—, cycloalkylene, or heterocyclylene, where each ring is substituted with R^{o} and R^{p} independently selected from hydrogen, deuterium, alkyl, alkoxy, halo, haloalkyl, haloalkoxy, cyano, and hydroxy, preferably hydrogen, deuterium, alkyl, alkoxy, hydroxy, cyano, halo, haloalkyl, and haloalkoxy;
Z⁵ is phenylene, monocyclic heteroarylene, or heterocycylene, where each ring is substituted with R^{q} and R^{r} independently selected from hydrogen, deuterium, alkyl, alkoxy, hydroxy, cyano, halo, haloalkyl, and haloalkoxy; and
Z⁶ is —S(O)₂—; and
wherein alkylene in Z⁴ is substituted with R^{s} and R^{t}.

34. The compound of any one of claims 1 to 26 and 30 to 33, or a pharmaceutically acceptable salt thereof, wherein:
X¹, X², X³, X⁴, Z¹, and Z² are each a bond;
Z³ is heterocyclylene, bridged heterocyclylene, or spiro heterocyclylene, where each ring is substituted with R^{m} and Rⁿ independently selected from hydrogen, deuterium, alkyl, alkoxy, hydroxy, cyano, halo, haloalkyl, and haloalkoxy;
Z⁴ is alkylene or —O—;
Z⁵ is phenylene or monocyclic heteroarylene, each ring substituted with R^{q} and R^{r} independently selected from hydrogen, deuterium, alkyl, alkoxy, hydroxy, cyano, halo, haloalkyl, and haloalkoxy; and
Z⁶ is —S(O)₂—; and
wherein alkylene in Z⁴ is substituted with R^{s} and R^{t}.

35. The compound of any one of claims 1 to 26 and 30, or a pharmaceutically acceptable salt thereof, wherein:
X¹, X², X³, X⁴, and Z¹ are each a bond;
Z² is cycloalkylene or heterocyclylene, where each ring is substituted with R^{j} and R^{k} independently selected from hydrogen, deuterium, alkyl, alkoxy, halo, haloalkyl, and haloalkoxy;
Z³ is cycloalkylene, phenylene, monocyclic heteroarylene, heterocyclylene, bicyclic heterocyclylene, bridged heterocyclylene, fused heterocyclylene, or spiro heterocyclylene, where each ring is substituted with R^{q} and R^{r} independently selected from hydrogen, deuterium, alkyl, alkoxy, hydroxy, cyano, halo, haloalkyl, and haloalkoxy;
Z⁴ is a bond, alkylene, or —O—;
Z⁵ is phenylene, monocyclic heteroarylene (e.g., pyridindiyl), or heterocycylene, where each ring is substituted with R^{q} and R^{r} independently selected from hydrogen, deuterium, alkyl, alkoxy, hydroxy, cyano, halo, haloalkyl, and haloalkoxy; and
Z⁶ is —S(O)₂—; and
wherein alkylene in Z⁴ is substituted with R^{s} and R^{t}.

36. The compound of any one of claims 1 to 26, or a pharmaceutically acceptable salt thereof, wherein:
X¹, X², X³, X⁴, and Z¹ are each a bond;
Z² is heterocyclylene substituted with R^{j} and R^{k}, preferably R^{j} and R^{k} are independently selected from hydrogen, deuterium, alkyl, alkoxy, halo, haloalkyl, and haloalkoxy;
Z³ is a bond, alkylene, or —O—;
Z⁴ is heterocyclylene, bridged heterocyclylene, or spiro heterocyclylene, where each ring is substituted with R^{o} and R^{p}, preferably R^{o} and R^{p} are independently selected from hydrogen, deuterium, alkyl, alkoxy, hydroxy, cyano, halo, haloalkyl, and haloalkoxy;
Z⁵ is phenylene or monocyclic heteroarylene, each ring substituted with R^{q} and R^{r}, preferably R^{q} and R^{r} are independently selected from hydrogen, deuterium, alkyl, alkoxy, hydroxy, cyano, halo, haloalkyl, and haloalkoxy; and
Z⁶ is —S(O)₂—; and
wherein alkylene in Z³ is substituted with R^{s} and R^{t}.

37. The compound of any one of claims 1 to 26, or a pharmaceutically acceptable salt thereof, wherein:
X¹, X², X³, X⁴, Z¹ and Z² are each a bond;
Z³ is alkylene, cycloalkylene, phenylene, -(alkylene)-phenylene-, -phenylene-(alkylene)-, monocyclic heteroarylene, -(alkylene)-monocyclic heteroarylene-, -monocyclic heteroarylene-(alkylene)-, heterocyclylene, -(alkylene)-heterocyclylene-, -heterocyclylene-(alkylene)-, bicyclic heterocyclylene, bridged heterocyclylene, -(alkylene)-bridged heterocyclylene-, -bridged heterocyclylene-(alkylene)-, fused heterocyclylene, spiro heterocyclylene, -(alkylene)-spiro heterocyclylene-, or -spiro heterocyclylene-(alkylene), where each ring, by itself or as part of another group, is substituted with R^{m} and Rⁿ;
Z⁴ is alkylene, -(alkylene-NR″)—, —(NR″-alkylene)-, —O—, —NR″—, —(O-alkylene)_{d}-, -(alkylene-O)_{d}—, cycloalkylene, -(alkylene)-cycloalkylene-, -cycloalkylene-(alkylene)-, spiro cyclolalkylene, phenylene, heteroarylene, heterocyclylene, -(alkylene)-heterocyclylene-, -heterocyclylene-(alkylene)-, fused heterocyclylene, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p};
Z⁵ is a bond, -alkylene, —NR″—, —O—, —C(O)—, —S(O)₂—, —NR′(CO)—, —C(O)NR—, phenylene, monocyclic heteroarylene, or heterocycylene, where each ring is substituted with R^{q} and R^{r}; and
Z⁶ is a bond, alkylene, —NR″—, —O—, -(alkylene-O)—, —C(O)—, —S(O)₂—, —NR′(CO)—, or —C(O)NR—; and
each alkylene in Z³, Z⁴, Z⁵, and Z⁶, itself or as part of another group, is independently substituted with R^{s} and R^{t}.

38. The compound of any one of claims 1 to 26, and 37, or a pharmaceutically acceptable salt thereof, wherein:
Z³ is alkylene, cycloalkylene, phenylene, -(alkylene)-phenylene-, -phenylene-(alkylene)-, monocyclic heteroarylene, -(alkylene)-monocyclic heteroarylene-, -monocyclic heteroarylene-(alkylene)-, heterocyclylene, -(alkylene)-heterocyclylene-, -heterocyclylene-(alkylene)-, bicyclic heterocyclylene, bridged heterocyclylene, -(alkylene)-bridged heterocyclylene-, -bridged heterocyclylene-(alkylene)-, fused heterocyclylene, spiro heterocyclylene, -(alkylene)-spiro heterocyclylene-, or -spiro heterocyclylene-(alkylene), where each ring, by itself or as part of another group, is substituted with R^{m} and Rⁿ;
Z⁴ is alkylene, -(alkylene-NR″)—, —(NR″-alkylene)-, —O—, —NR″—, —(O-alkylene)_{d}-, -(alkylene-O)_{d}—, cycloalkylene, -(alkylene)-cycloalkylene-, -cycloalkylene-(alkylene)-, spiro cyclolalkylene, phenylene, heteroarylene, heterocyclylene, -(alkylene)-heterocyclylene-, -heterocyclylene-(alkylene)-, fused heterocyclylene, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p};
Z⁵ is a bond, -alkylene, —NR″—, —O—, —C(O)—, —S(O)₂—, —NR′(CO)—, —C(O)NR—, phenylene, monocyclic heteroarylene, or heterocycylene, where each ring is substituted with R^{q} and R^{r}; and
Z⁶ is —S(O)₂—; and
and each alkylene in Z³, Z⁴, and Z⁵, itself or as part of another group, is independently substituted with R^{s} and R^{t}.

39. The compound of any one of claims 1 to 26, 37, and 38, or a pharmaceutically acceptable salt thereof, wherein:
Z³ is alkylene, phenylene, -(alkylene)-phenylene-, -phenylene-(alkylene)-, monocyclic heteroarylene, -(alkylene)-monocyclic heteroarylene-, -monocyclic heteroarylene-(alkylene)-, heterocyclylene, -(alkylene)-heterocyclylene-, -heterocyclylene-(alkylene)-, bridged heterocyclylene, -(alkylene)-bridged heterocyclylene-, -bridged heterocyclylene-(alkylene)-, spiro heterocyclylene, -(alkylene)-spiro heterocyclylene-, or -spiro heterocyclylene-(alkylene), where each ring, by itself or as part of another group, is substituted with R^{m} and Rⁿ;
Z⁴ is alkylene, -(alkylene-NR″)—, —(NR″-alkylene)-, —O—, —NR″—, —(O-alkylene)_{d}-, -(alkylene-O)_{d}—, cycloalkylene, -(alkylene)-cycloalkylene-, -cycloalkylene-(alkylene)-, spiro cyclolalkylene, phenylene, heteroarylene, heterocyclylene, -(alkylene)-heterocyclylene-, -heterocyclylene-(alkylene)-, fused heterocyclylene, bridged heterocyclylene, -(alkylene)-bridged heterocyclylene-, -bridged heterocyclylene-(alkylene)-, spiro heterocyclylene, -(alkylene)-spiro heterocyclylene, or -spiro heterocyclylene-(alkylene)-, where each ring, by itself or as part of another group, is substituted with R^{o} and R^{p};
Z⁵ is phenylene, monocyclic heteroarylene, or heterocycylene, where each ring is substituted with R^{q} and R^{r}; and
Z⁶ is —S(O)₂—; and
each alkylene in Z³ and Z⁴, itself or as part of another group, is independently substituted with R^{s} and R^{t}.

40. The compound of any one of claims 1 to 39, or a pharmaceutically acceptable salt thereof, wherein —Z⁵— is 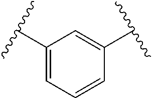 [SMILES: *c1cccc(*)c1]
(i.e., Z⁵ is phenylene where Z⁴ and Z⁶ are attached at meta position of the phenylene ring) substituted with R^{q} and R^{r} independently selected from hydrogen, deuterium, alkyl, alkoxy, halo, haloalkyl, and haloalkoxy.

41. The compound of any one of claims 1 to 40, or a pharmaceutically acceptable salt thereof, wherein —Z⁵— is

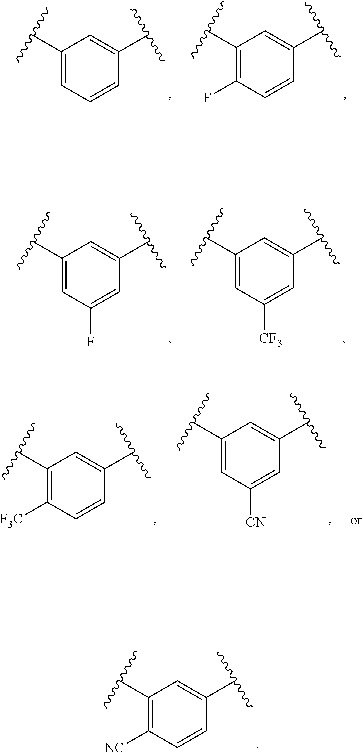

42. The compound of any one of claims 1 to 39, or a pharmaceutically acceptable salt thereof, wherein —Z⁵— is monocyclic heteroarylene substituted with R^{q} and R^{r} independently selected from hydrogen, alkyl, alkoxy, halo, haloalkyl, and haloalkoxy.

43. The compound of any one of claims 1 to 42, or a pharmaceutically acceptable salt thereof, wherein each heterocyclylene, bridged heterocyclylene, and spiro heterocyclylene, of Z¹, Z², Z³, Z⁴, and Z⁵, when present, are independently selected from: 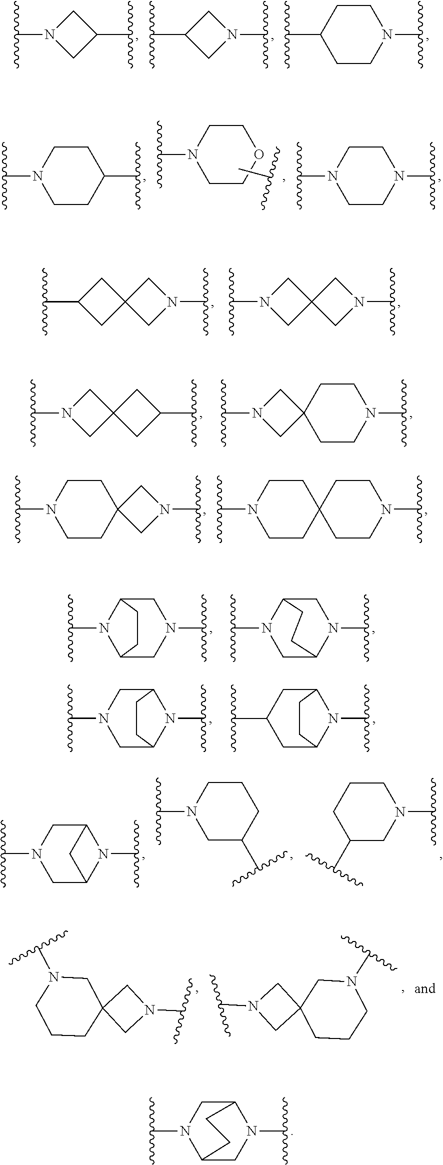
wherein each ring is optionally substituted with 1, 2, or 3 fluoro, unless stated otherwise in any of the claims above.

44. The compound of any one of claims 1 to 35 and 37 to 39, or a pharmaceutically acceptable salt thereof, wherein —Z³—Z⁴—Z⁵—Z⁶— is: 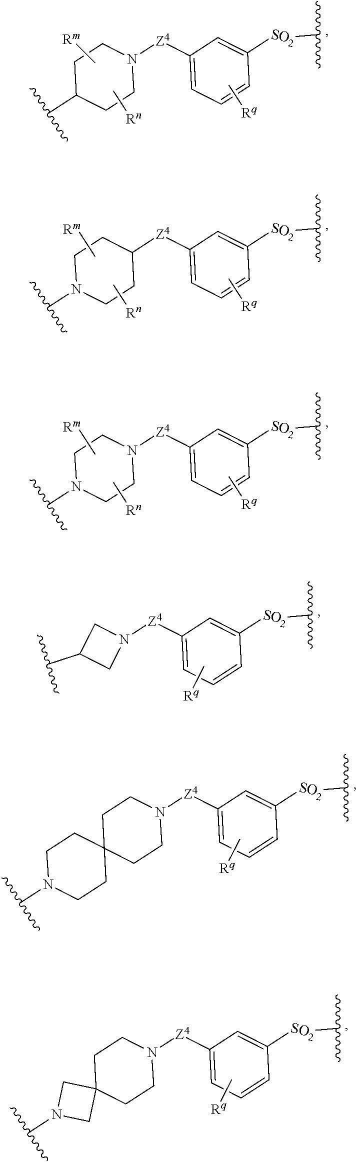 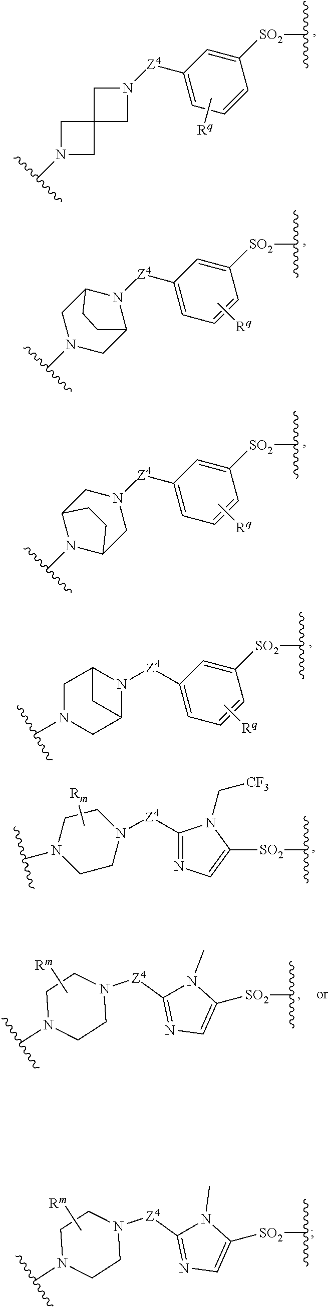
wherein each R^{m}, Rⁿ, and R^{q} are independently selected from hydrogen, alkyl, halo, haloalkyl, haloalkoxy, alkoxy, and cyano.

45. The compound of any one of claims 1 to 35, 37 to 42 and 44, or a pharmaceutically acceptable salt thereof, wherein Z⁴ is alkylene substituted with R^{s} and R^{t} where R^{s} and R^{t} are hydrogen.

46. The compound of any one of claims 1 to 35, 37 to 42 and 44, or a pharmaceutically acceptable salt thereof, wherein Z⁴ is —O—.

47. The compound of any one of claims 1 to 35, 37 to 42 and 44, or a pharmaceutically acceptable salt thereof, wherein Z⁴ is alkylene substituted with R^{s} and R^{t} where R^{s} is hydrogen or deuterium and R^{t} is hydrogen, deuterium, haloalkyl, hydroxy, alkoxy, or cyano.

48. The compound of any one of 1 to 29, 37 to 42 and 44, or a pharmaceutically acceptable salt thereof, wherein Z⁴ is -(alkylene)-heterocyclylene- where heterocyclylene is substituted with R^{o} and R^{p}.

49. The compound of any one of 1 to 29, 37 to 42, 44, and 48 or a pharmaceutically acceptable salt thereof, wherein Z⁴ is —(CH₂)-heterocyclylene- where heterocyclylene is substituted with R^{o} and R^{p}.

50. The compound of any one of 1 to 29, 37 to 42, 44, 48, and 49 or a pharmaceutically acceptable salt thereof, wherein Z⁴ is:

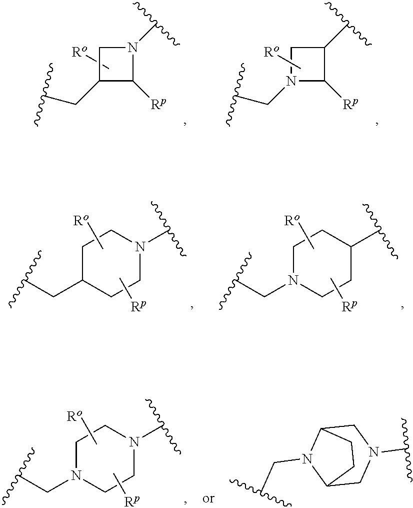

51. The compound of any one of 1 to 29, 37 to 42, 44, and 48 to 50 or a pharmaceutically acceptable salt thereof, wherein —Z³—Z⁴—Z⁵—Z⁶— is:

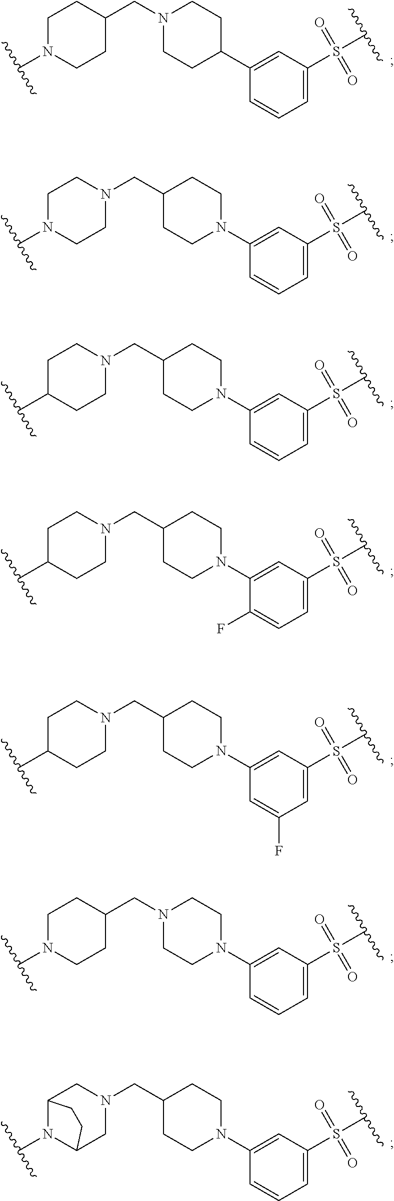

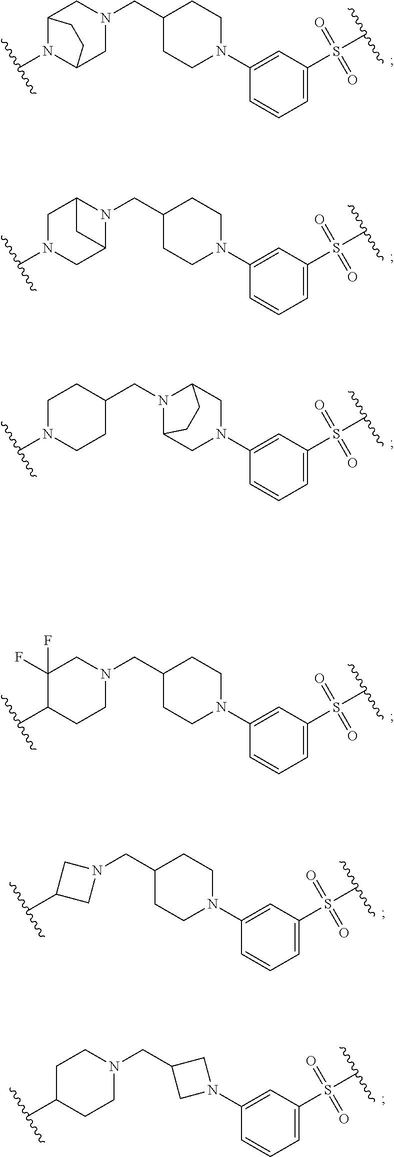 [SMILES: *N1C2CCC1CN(CC1CCN(c3cccc(S(*)(=O)=O)c3)CC1)C2 ; *N1CC2CC(C1)N2CC1CCN(c2cccc(S(*)(=O)=O)c2)CC1 ; *N1CCC(CN2C3CCC2CN(c2cccc(S(*)(=O)=O)c2)C3)CC1 ; *C1CCN(CC2CCN(c3cccc(S(*)(=O)=O)c3)CC2)CC1(F)F ; *C1CN(CC2CCN(c3cccc(S(*)(=O)=O)c3)CC2)C1 ; *C1CCN(CC2CN(c3cccc(S(*)(=O)=O)c3)C2)CC1]

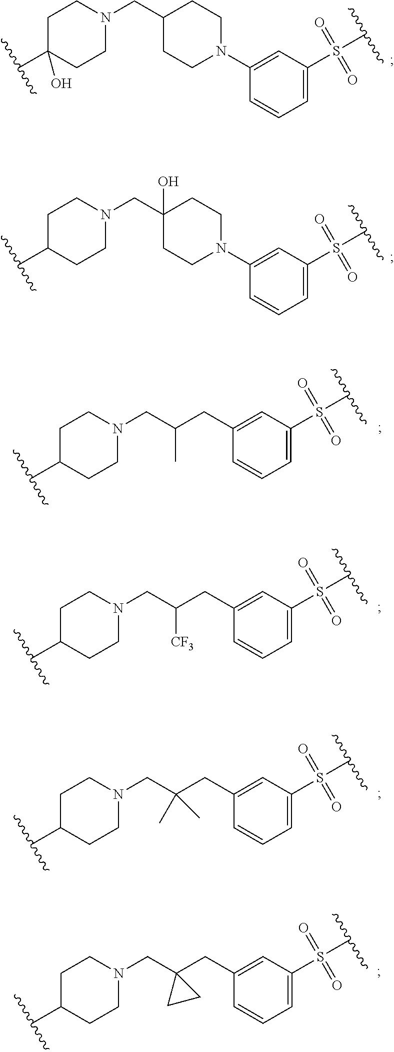 [SMILES: *C1(O)CCN(CC2CCN(c3cccc(S(*)(=O)=O)c3)CC2)CC1 ; *C1CCN(CC2(O)CCN(c3cccc(S(*)(=O)=O)c3)CC2)CC1 ; *C1CCN(CC(C)Cc2cccc(S(*)(=O)=O)c2)CC1 ; *C1CCN(CC(Cc2cccc(S(*)(=O)=O)c2)C(F)(F)F)CC1 ; *C1CCN(CC(C)(C)Cc2cccc(S(*)(=O)=O)c2)CC1 ; *C1CCN(CC2(Cc3cccc(S(*)(=O)=O)c3)CC2)CC1]

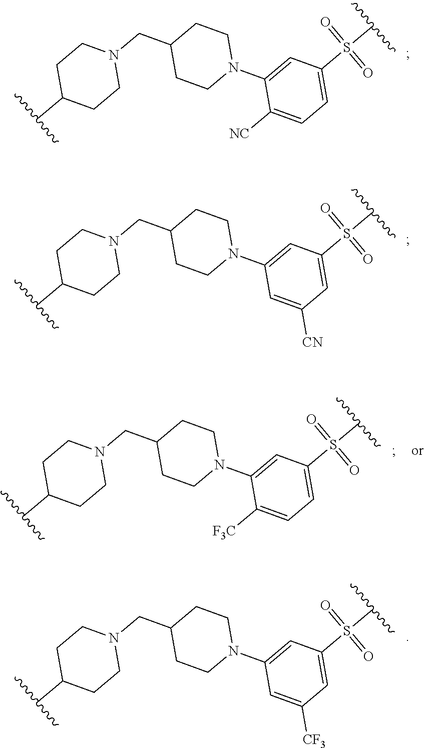 [SMILES: *C1CCN(CC2CCN(c3cc(S(*)(=O)=O)ccc3C#N)CC2)CC1 ; *C1CCN(CC2CCN(c3cc(C#N)cc(S(*)(=O)=O)c3)CC2)CC1 ; *C1CCN(CC2CCN(c3cc(S(*)(=O)=O)ccc3C(F)(F)F)CC2)CC1 ; *C1CCN(CC2CCN(c3cc(C(F)(F)F)cc(S(*)(=O)=O)c3)CC2)CC1]

52. The compound of any one of claims 1 to 51, or a pharmaceutically acceptable salt thereof, wherein Degron is the E3 ubiquitin ligase ligand selected from: 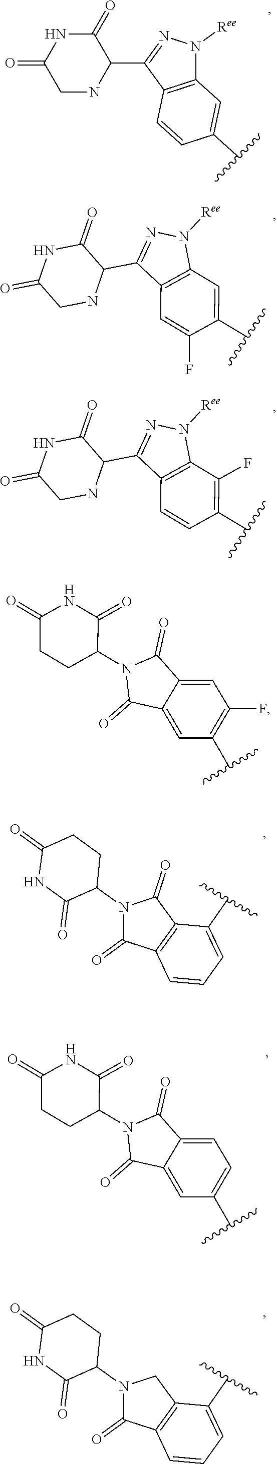 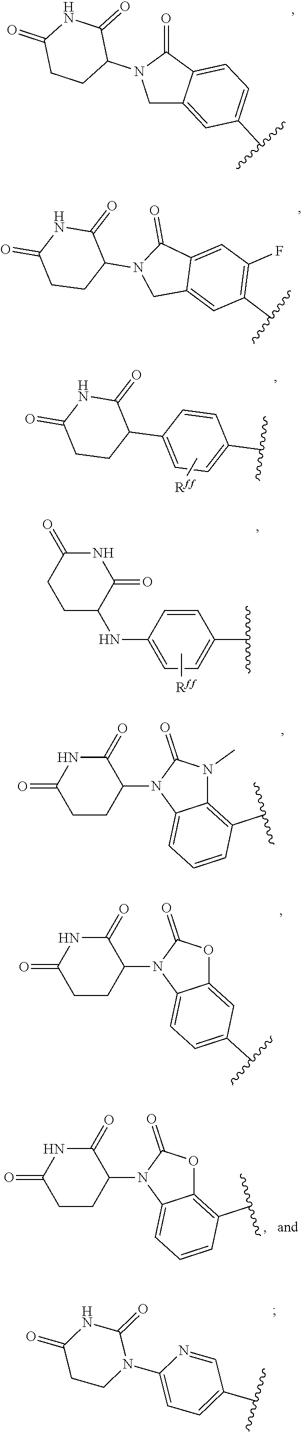
where each R^{ee} is hydrogen, methyl, ethyl, cyclopropyl, or 2,2,2-trifluoroethyl and each R^{ff} is hydrogen, methyl, cyclopropyl, fluoro, cyano, methoxy, difluoromethoxy, trifluoromethoxy, or trifluoromethyl.

53. The compound of claim 1, or a pharmaceutically acceptable salt thereof, wherein the compound is selected from:
1-(1-methyl-6-(1-(3-((4-((5-(trifluoromethyl)pyrimidin-2-yl)amino)piperidin-1-yl)sulfonyl)benzyl)-piperidin-4-yl)-1H-indazol-3-yl)dihydropyrimidine-2,4(1H,3H)-dione;
1-(7-fluoro-1-methyl-6-(1-(3-((4-((5-(trifluoromethyl)pyrimidin-2-yl)amino)piperidin-1-yl)sulfonyl)benzyl)-piperidin-4-yl)-1H-indazol-3-yl)dihydropyrimidine-2,4(1H,3H)-dione;
1-(6-(3,3-difluoro-1-(3-((4-((5-(trifluoromethyl)pyrimidin-2-yl)amino)-piperidin-1-yl)sulfonyl)benzyl)-piperidin-4-yl)-1-methyl-1H-indazol-3-yl)dihydropyrimidine-2,4(1H,3H)-dione;
1-(1-methyl-6-(6-(3-((4-((5-(trifluoromethyl)pyrimidin-2-yl)amino)piperidin-1-yl)sulfonyl)benzyl)-2,6-diazaspiro[3.3]heptan-2-yl)-1H-indazol-3-yl)dihydropyrimidine-2,4(1H,3H)-dione;
1-(1-(2,2,2-trifluoroethyl)-6-(1-(3-((4-((5-(trifluoromethyl)pyrimidin-2-yl)amino)-piperidin-1-yl)sulfonyl)benzyl)-piperidin-4-yl)-1H-indazol-3-yl)dihydropyrimidine-2,4(1H,3H)-dione;
1-(1-methyl-6-(1-(3-((4-((5-(trifluoromethyl)pyrimidin-2-yl)amino)-piperidin-1-yl)sulfonyl)phenethyl)piperidin-4-yl)-1H-indazol-3-yl)dihydropyrimidine-2,4(1H,3H)-dione;
N—((R)-1-((2S,4R)-4-hydroxy-2-(((S)-1-(4-(4-methylthiazol-5-yl)phenyl)ethyl)-carbamoyl) pyrrolidin-1-yl)-3,3-dimethyl-1-oxobutan-2-yl)-1-(3-((4-((5-(trifluoro-methyl)pyrimidin-2-yl)amino)piperidin-1-yl)sulfonyl)benzyl)piperidine-4-carboxamide;
(2S,4R)-1-((R)-2-(1-fluorocyclopropane-1-carboxamido)-3-methyl-3-(((1-(3-((4-((5-(trifluoromethyl)pyrimidin-2-yl)amino)-piperidin-1-yl)sulfonyl)benzyl)piperidin-4-yl)methyl)thio)butanoyl)-4-hydroxy-N—((S)-1-(4-(4-methylthiazol-5-yl)phenyl)-ethyl) pyrrolidine-2-carboxamide;
(2S,4R)-1-((S)-3,3-dimethyl-2-(2-(1-(3-((4-((5-(trifluoromethyl)pyrimidin-2-yl)amino)piperidin-1-yl)sulfonyl)-benzyl)piperidin-4-yl) acetamido) butanoyl)-4-hydroxy-N—((S)-1-(4-(4-methylthiazol-5-yl)phenyl)ethyl) pyrrolidine-2-carboxamide;
1-(1-methyl-6-(1-(3-(((1r,4r)-4-((5-(trifluoromethyl)pyrimidin-2-yl)amino)-cyclohexyl)sulfonyl)benzyl)piperidin-4-yl)-1H-indazol-3-yl)dihydropyrimidine-2,4(1H,3H)-dione;
1-(6-(1-(3-((4-((5-chloropyrimidin-2-yl)amino)piperidin-1-yl)sulfonyl)benzyl)-piperidin-4-yl)-1-methyl-1H-indazol-3-yl)dihydropyrimidine-2,4(1H,3H)-dione;
1-(1-methyl-6-(4-(3-((4-((5-(trifluoro-methyl)pyrimidin-2-yl)amino)piperidin-1-yl)sulfonyl)benzyl)piperazin-1-yl)-1H-indazol-3-yl)dihydropyrimidine-2,4(1H,3H)-dione;
1-(6-(1-(3-((4-((5-(difluoromethyl)-pyrimidin-2-yl)amino)piperidin-1-yl)sulfonyl)benzyl)piperidin-4-yl)-1-methyl-1H-indazol-3-yl)dihydropyrimidine-2,4(1H,3H)-dione;
1-(6-(1-(3-(((3R,4S)-3-fluoro-4-((5-(trifluoromethyl)pyrimidin-2-yl)amino)-piperidin-1-yl)sulfonyl)benzyl)-piperidin-4-yl)-1-methyl-1H-indazol-3-yl)dihydropyrimidine-2,4(1H,3H)-dione;
1-(1-methyl-6-(1-((1-((4-((5-(trifluoro-methyl)pyrimidin-2-yl)amino)piperidin-1-yl)sulfonyl)piperidin-4-yl)methyl)piperidin-4-yl)-1H-indazol-3-yl)dihydropyrimidine-2,4(1H,3H)-dione;
1-(5-fluoro-1-methyl-6-(1-(3-((4-((5-(trifluoromethyl)pyrimidin-2-yl)amino)-piperidin-1-yl)sulfonyl)benzyl)piperidin-4-yl)-1H-indazol-3-yl)dihydropyrimidine-2,4(1H,3H)-dione;
1-(6-(1-(3-((4-((5-(1,1-difluoroethyl)-pyrimidin-2-yl)amino)piperidin-1-yl)sulfonyl)benzyl)piperidin-4-yl)-1-methyl-1H-indazol-3-yl)dihydropyrimidine-2,4(1H,3H)-dione;
1-(6-(1-((1-((3-fluoro-4-((5-(trifluoro-methyl)pyrimidin-2-yl)amino)phenyl)-sulfonyl)piperidin-4-yl)methyl)piperidin-4-yl)-1-methyl-1H-indazol-3-yl)dihydropyrimidine-2,4(1H,3H)-dione;
1-(1-methyl-6-(1-((1-(((1r,4r)-4-((5-(trifluoromethyl)pyrimidin-2-yl)amino)-cyclohexyl)sulfonyl)piperidin-3-yl)methyl)piperidin-4-yl)-1H-indazol-3-yl)dihydro-pyrimidine-2,4(1H,3H)-dione;
1-(1-methyl-6-(1-((1-(((1r,4r)-4-((5-(trifluoromethyl)pyrimidin-2-yl)amino)-cyclohexyl)sulfonyl)piperidin-4-yl)methyl)-piperidin-4-yl)-1H-indazol-3-yl)-dihydropyrimidine-2,4(1H,3H)-dione;
1-(6-(1-(3-((4-((5-chloropyrimidin-2-yl)amino)piperidin-1-yl)sulfonyl)benzyl)-piperidin-4-yl)-1-(2,2,2-trifluoroethyl)-1H-indazol-3-yl)dihydropyrimidine-2,4(1H,3H)-dione;
5-(4-(3-((4-((5-chloropyrimidin-2-yl)amino)piperidin-1-yl)sulfonyl)benzyl)-piperazin-1-yl)-2-(2,6-dioxopiperidin-3-yl)-6-fluoroisoindoline-1,3-dione;
1-(6-(1-(3-((4-((5-chloropyrimidin-2-yl)amino)piperidin-1-yl)sulfonyl)benzyl)-piperidin-4-yl)-5-fluoro-1-methyl-1H-indazol-3-yl)dihydropyrimidine-2,4(1H,3H)-dione;
1-(6-(1-(3-((4-((5-bromopyrimidin-2-yl)amino)piperidin-1-yl)sulfonyl)benzyl)-piperidin-4-yl)-1-methyl-1H-indazol-3-yl)dihydropyrimidine-2,4(1H,3H)-dione;
1-(6-(4-(3-((4-((5-chloropyrimidin-2-yl)amino)piperidin-1-yl)sulfonyl)benzyl)-piperazin-1-yl)-1-methyl-1H-indazol-3-yl)dihydropyrimidine-2,4(1H,3H)-dione;
1-(6-(1-(3-((4-((5-fluoropyrimidin-2-yl)amino)piperidin-1-yl)sulfonyl)benzyl)-piperidin-4-yl)-1-methyl-1H-indazol-3-yl)dihydropyrimidine-2,4(1H,3H)-dione;
1-(6-(1-(3-(((3R,4S)-4-((5-chloropyrimidin-2-yl)amino)-3-fluoropiperidin-1-yl)sulfonyl)benzyl)piperidin-4-yl)-1-methyl-1H-indazol-3-yl)dihydropyrimidine-2,4(1H,3H)-dione;
1-(6-(1-(3-((4-((5-chloropyrimidin-2-yl)amino)piperidin-1-yl)sulfonyl)benzyl)-piperidin-4-yl)-7-fluoro-1-methyl-1H-indazol-3-yl)dihydropyrimidine-2,4(1H,3H)-dione;
1-(1-methyl-6-(1-(2-methyl-3-(3-((4-((5-(trifluoromethyl)pyrimidin-2-yl)amino)-piperidin-1-yl)sulfonyl)phenyl)-propyl)piperidin-4-yl)-1H-indazol-3-yl)dihydropyrimidine-2,4(1H,3H)-dione;
1-(6-(1-(1-(3-((4-((5-chloropyrimidin-2-yl)amino)piperidin-1-yl)sulfonyl)phenyl)-2,2-difluoroethyl)piperidin-4-yl)-1-methyl-1H-indazol-3-yl)dihydropyrimidine-2,4(1H,3H)-dione;
1-(1-methyl-6-(1-(2-(1-((4-((5-(trifluoromethyl)pyrimidin-2-yl)amino)-piperidin-1-yl)sulfonyl)piperidin-3-yl)ethyl)piperidin-4-yl)-1H-indazol-3-yl)dihydropyrimidine-2,4(1H,3H)-dione;
1-(6-(1-(3-((4-((5-chloropyrimidin-2-yl)amino)piperidin-1-yl)sulfonyl)benzyl)-3,3-difluoropiperidin-4-yl)-1-methyl-1H-indazol-3-yl)dihydropyrimidine-2,4(1H,3H)-dione;
3-(5-(4-(3-((4-((5-chloropyrimidin-2-yl)amino)piperidin-1-yl)sulfonyl)benzyl)-piperazin-1-yl)-6-fluoro-1-oxoisoindolin-2-yl)piperidine-2,6-dione;
1-(6-(1-(3-((4-((5-(difluoromethoxy)-pyrimidin-2-yl)amino)piperidin-1-yl)sulfonyl)-benzyl)piperidin-4-yl)-1-methyl-1H-indazol-3-yl)dihydropyrimidine-2,4(1H,3H)-dione;
1-(1-methyl-6-(1-(3-(1-((4-((5-(trifluoro-methyl)pyrimidin-2-yl)amino)piperidin-1-yl)sulfonyl)piperidin-4-yl)propyl)piperidin-4-yl)-1H-indazol-3-yl)dihydropyrimidine-2,4(1H,3H)-dione;
1-(6-(1-(1-(3-((4-((5-chloropyrimidin-2-yl)amino)piperidin-1-yl)sulfonyl)phenyl)-ethyl)piperidin-4-yl)-1-methyl-1H-indazol-3-yl)dihydropyrimidine-2,4(1H,3H)-dione;
1-(6-(1-((1-(3-((4-((5-chloropyrimidin-2-yl)amino)piperidin-1-yl)sulfonyl)phenyl)-piperidin-4-yl)methyl)piperidin-4-yl)-1-methyl-1H-indazol-3-yl)dihydropyrimidine-2,4(1H,3H)-dione;
1-(1-methyl-6-(1-((1-(3-((4-((5-(trifluoro-methyl)pyrimidin-2-yl)amino)piperidin-1-yl)sulfonyl)phenyl)piperidin-4-yl)methyl)piperidin-4-yl)-1H-indazol-3-yl)dihydropyrimidine-2,4(1H,3H)-dione;
3-(1-oxo-5-(4-(3-((4-((5-(trifluoromethyl)-pyrimidin-2-yl)amino)piperidin-1-yl)sulfonyl)phenoxy)piperidin-1-yl)isoindolin-2-yl)piperidine-2,6-dione;
3-(6-fluoro-1-oxo-5-(4-(3-((4-((5-(trifluoro-methyl)pyrimidin-2-yl)amino)piperidin-1-yl)sulfonyl)benzyl)piperazin-1-yl)isoindolin-2-yl)piperidine-2,6-dione;
3-(5-(4-(3-((4-((5-(difluoromethoxy)-pyrimidin-2-yl)amino)piperidin-1-yl)sulfonyl)-benzyl)piperazin-1-yl)-6-fluoro-1-oxoisoindolin-2-yl)piperidine-2,6-dione;
3-(1-oxo-5-(4-(3-(3-((4-((5-(trifluoro-methyl)pyrimidin-2-yl)amino)piperidin-1-yl)sulfonyl)phenoxy)azetidin-1-yl)piperidin-1-yl)isoindolin-2-yl)piperidine-2,6-dione;
1-(6-(6-(3-((4-((5-chloropyrimidin-2-yl)amino)piperidin-1-yl)sulfonyl)benzyl)-2,6-diazaspiro[3.3]heptan-2-yl)-1-methyl-1H-indazol-3-yl)dihydropyrimidine-2,4(1H,3H)-dione;
1-(1-methyl-6-(1-((1-methyl-5-((4-((5-(trifluoromethyl)pyrimidin-2-yl)amino)-piperidin-1-yl)sulfonyl)-1H-imidazol-2-yl)methyl)piperidin-4-yl)-1H-indazol-3-yl)dihydro-pyrimidine-2,4(1H,3H)-dione;
3-(1-oxo-5-(1-(3-((4-((5-(trifluoromethyl)-pyrimidin-2-yl)amino)piperidin-1-yl)sulfonyl)benzyl)azetidin-3-yl)isoindolin-2-yl)piperidine-2,6-dione;
1-(1-methyl-6-(1-(3-((4-((5-(trifluoro-methyl)pyrimidin-2-yl)amino)piperidin-1-yl)sulfonyl)benzyl)azetidin-3-yl)-1H-indazol-3-yl)dihydropyrimidine-2,4(1H,3H)-dione;
3-(5-(1-(3-((4-((5-chloropyrimidin-2-yl)amino)piperidin-1-yl)sulfonyl)benzyl)-azetidin-3-yl)-1-oxoisoindolin-2-yl)piperidine-2,6-dione;
1-(1-methyl-6-(4-((1-(3-((4-((5-(trifluoro-methyl)pyrimidin-2-yl)amino)piperidin-1-yl)sulfonyl)phenyl)piperidin-4-yl)methyl)piperazin-1-yl)-1H-indazol-3-yl)dihydropyrimidine-2,4(1H,3H)-dione;
1-(6-(4-((1-(3-((4-((5-chloropyrimidin-2-yl)amino)piperidin-1-yl)sulfonyl)phenyl)-piperidin-4-yl)methyl)piperazin-1-yl)-1-methyl-1H-indazol-3-yl)dihydropyrimidine-2,4(1H,3H)-dione;
1-(6-(1-(3-(3-((4-((5-chloropyrimidin-2-yl)amino)piperidin-1-yl)sulfonyl)phenyl)-2-methylpropyl)piperidin-4-yl)-1-methyl-1H-indazol-3-yl)dihydropyrimidine-2,4(1H,3H)-dione;
1-(6-(1-(3-(3-((4-((5-chloropyrimidin-2-yl)amino)piperidin-1-yl)sulfonyl)phenyl)-2,2-dimethylpropyl)piperidin-4-yl)-1-methyl-1H-indazol-3-yl)dihydropyrimidine-2,4(1H,3H)-dione;
1-(6-(1-(2,2-dimethyl-3-(3-((4-((5-(trifluoro-methyl)pyrimidin-2-yl)amino)piperidin-1-yl)sulfonyl)phenyl)propyl)piperidin-4-yl)-1-methyl-1H-indazol-3-yl)dihydropyrimidine-2,4(1H,3H)-dione;
1-(6-(1-(3-((4-((5-chloropyrimidin-2-yl)amino)piperidin-1-yl)sulfonyl)benzyl)-azetidin-3-yl)-1-methyl-1H-indazol-3-yl)dihydropyrimidine-2,4(1H,3H)-dione;
1-(6-(1-(3-((4-((5-methoxypyrimidin-2-yl)amino)piperidin-1-yl)sulfonyl)benzyl)-piperidin-4-yl)-1-methyl-1H-indazol-3-yl)dihydropyrimidine-2,4(1H,3H)-dione;
1-(1-methyl-6-(1-(3-((4-((5-vinylpyrimidin-2-yl)amino)piperidin-1-yl)sulfonyl)benzyl)-piperidin-4-yl)-1H-indazol-3-yl)dihydropyrimidine-2,4(1H,3H)-dione;
1-(6-(1-(3-(((1r,4r)-4-((5-chloropyrimidin-2-yl)amino)cyclohexyl)sulfonyl)-benzyl)piperidin-4-yl)-1-methyl-1H-indazol-3-yl)dihydropyrimidine-2,4(1H,3H)-dione;
1-(1-methyl-6-(1-(3-((4-((5-methyl-pyrimidin-2-yl)amino)piperidin-1-yl)sulfonyl)benzyl)piperidin-4-yl)-1H-indazol-3-yl)dihydropyrimidine-2,4(1H,3H)-dione;
1-(6-(1-(3-((4-((5-ethylpyrimidin-2-yl)amino)piperidin-1-yl)sulfonyl)benzyl)-piperidin-4-yl)-1-methyl-1H-indazol-3-yl)dihydropyrimidine-2,4(1H,3H)-dione;
1-(6-(4-(3-((4-((5-chloropyrimidin-2-yl)amino)piperidin-1-yl)sulfonyl)benzyl)-piperazin-1-yl)-1-(2,2,2-trifluoroethyl)-1H-indazol-3-yl)dihydropyrimidine-2,4(1H,3H)-dione;
1-(6-(1-(4-((4-((5-chloropyrimidin-2-yl)amino)piperidin-1-yl)sulfonyl)benzyl)-piperidin-4-yl)-1-methyl-1H-indazol-3-yl)dihydropyrimidine-2,4(1H,3H)-dione;
1-(1-methyl-6-(1-(4-((4-((5-(trifluoro-methyl)pyrimidin-2-yl)amino)piperidin-1-yl)sulfonyl)benzyl)piperidin-4-yl)-1H-indazol-3-yl)dihydropyrimidine-2,4(1H,3H)-dione;
1-(1-methyl-6-(1-(3-((4-((5-phenoxy-pyrimidin-2-yl)amino)piperidin-1-yl)sulfonyl)benzyl)piperidin-4-yl)-1H-indazol-3-yl)dihydropyrimidine-2,4(1H,3H)-dione;
1-(6-(1-(3-(3-((4-((5-(difluoromethoxy)-pyrimidin-2-yl)amino)piperidin-1-yl)sulfonyl)phenyl)-2-methylpropyl)-piperidin-4-yl)-1-methyl-1H-indazol-3-yl)dihydro-pyrimidine-2,4(1H,3H)-dione;
1-(6-(4-(3-(((1r,4r)-4-((5-chloropyrimidin-2-yl)amino)cyclohexyl)sulfonyl)benzyl)-piperazin-1-yl)-1-methyl-1H-indazol-3-yl)dihydropyrimidine-2,4(1H,3H)-dione;
1-(6-(1-(3-((4-((5-(difluoromethoxy)-pyrimidin-2-yl)amino)piperidin-1-yl)sulfonyl)benzyl)piperidin-4-yl)-1-(2,2,2-trifluoroethyl)-1H-indazol-3-yl)dihydro-pyrimidine-2,4(1H,3H)-dione;
1-(6-(1-(3-((4-((5-(dimethylamino)-pyrimidin-2-yl)amino)piperidin-1-yl)-sulfonyl)benzyl)piperidin-4-yl)-1-methyl-1H-indazol-3-yl)dihydropyrimidine-2,4(1H,3H)-dione;
1-(7-chloro-6-(1-((1-(3-((4-((5-chloro-pyrimidin-2-yl)amino)piperidin-1-yl)sulfonyl)phenyl)piperidin-4-yl)methyl)-piperidin-4-yl)-1-methyl-1H-indazol-3-yl)dihydropyrimidine-2,4(1H,3H)-dione;
2-(2,6-dioxopiperidin-3-yl)-5-fluoro-6-(4-(3-((4-((5-(trifluoromethyl)pyrimidin-2-yl)amino)piperidin-1-yl)sulfonyl)benzyl)-piperazin-1-yl)isoindoline-1,3-dione;
1-(1-methyl-6-(1-(3-((4-((5-(trifluoro-methyl)pyrimidin-2-yl)amino)piperidin-1-yl)sulfonyl)benzyl)azetidin-3-yl)-1H-indazol-3-yl)dihydropyrimidine-2,4(1H,3H)-dione;
3-(5-(4-(3-((4-((5-(difluoromethoxy)-pyrimidin-2-yl)amino)piperidin-1-yl)sulfonyl)benzyl)piperazin-1-yl)-6-fluoro-1-oxoisoindolin-2-yl)piperidine-2,6-dione;
1-(6-(1-(3-((4-((5-(difluoromethoxy)-pyrimidin-2-yl)amino)piperidin-1-yl)sulfonyl)benzyl)-3,3-difluoropiperidin-4-yl)-1-methyl-1H-indazol-3-yl)dihydropyrimidine-2,4(1H,3H)-dione;
3-(1-oxo-5-(4-(3-((4-((5-(trifluoromethyl)-pyrimidin-2-yl)amino)piperidin-1-yl)sulfonyl)benzyl)piperazin-1-yl)isoindolin-2-yl)piperidine-2,6-dione;
1-(6-(1-(3-((4-((5-(difluoromethoxy)-pyrimidin-2-yl)amino)piperidin-1-yl)sulfonyl)benzyl)piperidin-4-yl)-5-fluoro-1-methyl-1H-indazol-3-yl)dihydropyrimidine-2,4(1H,3H)-dione;
3-((4-(4-(3-((4-((5-(trifluoromethyl)-pyrimidin-2-yl)amino)piperidin-1-yl)sulfonyl)benzyl)piperazin-1-yl)phenyl)amino)piperidine-2,6-dione;
5-(4-((1-(3-((4-((5-chloropyrimidin-2-yl)amino)piperidin-1-yl)sulfonyl)phenyl)-piperidin-4-yl)methyl)piperazin-1-yl)-2-(2,6-dioxopiperidin-3-yl)-6-fluoroisoindoline-1,3-dione;
2-(2,6-dioxopiperidin-3-yl)-5-fluoro-6-(4-((1-(3-((4-((5-(trifluoromethyl)pyrimidin-2-yl)amino)piperidin-1-yl)sulfonyl)phenyl)-piperidin-4-yl)methyl)piperazin-1-yl)isoindoline-1,3-dione;
1-(6-(1-(3-((4-((5-(difluoromethoxy)-pyrimidin-2-yl)amino)piperidin-1-yl)sulfonyl)benzyl)azetidin-3-yl)-1-methyl-1H-indazol-3-yl)dihydropyrimidine-2,4(1H,3H)-dione;
1-(4-(4-(3-((4-((5-(trifluoromethyl)-pyrimidin-2-yl)amino)piperidin-1-yl)sulfonyl)-benzyl)piperazin-1-yl)phenyl)dihydropyrimidine-2,4(1H,3H)-dione;
3-(3-methyl-2-oxo-4-(1-(3-((4-((5-(trifluoromethyl)pyrimidin-2-yl)amino)piperidin-1-yl)sulfonyl)benzyl)-piperidin-4-yl)-2,3-dihydro-1H-benzo[d]imidazol-1-yl)piperidine-2,6-dione;
1-(6-(4-((3-((4-((5-chloropyrimidin-2-yl)amino)piperidin-1-yl)sulfonyl)benzyl)-amino)piperidin-1-yl)-1-methyl-1H-indazol-3-yl)dihydropyrimidine-2,4(1H,3H)-dione;
1-(1-methyl-6-(4-((3-((4-((5-(trifluoro-methyl)pyrimidin-2-yl)amino)piperidin-1-yl)sulfonyl)benzyl)amino)piperidin-1-yl)-1H-indazol-3-yl)dihydropyrimidine-2,4(1H,3H)-dione;
1-(6-(1-(3-(3-((4-((5-(difluoromethoxy)-pyrimidin-2-yl)amino)piperidin-1-yl)sulfonyl)-phenyl)propyl)piperidin-4-yl)-1-methyl-1H-indazol-3-yl)dihydropyrimidine-2,4(1H,3H)-dione;
1-(1-methyl-6-(1-(3-(3-((4-((5-(trifluoro-methyl)pyrimidin-2-yl)amino)piperidin-1-yl)sulfonyl)phenyl)propyl)piperidin-4-yl)-1H-indazol-3-yl)dihydropyrimidine-2,4(1H,3H)-dione;
1-(1-methyl-6-(4-((methyl (3-((4-((5-(trifluoromethyl)pyrimidin-2-yl)amino)-piperidin-1-yl)sulfonyl)benzyl)amino)-methyl)piperidin-1-yl)-1H-indazol-3-yl)dihydropyrimidine-2,4(1H,3H)-dione;
1-(7-chloro-1-methyl-6-(1-((1-(3-((4-((5-(trifluoromethyl)pyrimidin-2-yl)amino)-piperidin-1-yl)sulfonyl)phenyl)piperidin-4-yl)methyl)piperidin-4-yl)-1H-indazol-3-yl)dihydropyrimidine-2,4(1H,3H)-dione;
1-(6-(3,3-difluoro-1-((1-(3-((4-((5-(trifluoromethyl)pyrimidin-2-yl)amino)-piperidin-1-yl)sulfonyl)phenyl)piperidin-4-yl)methyl)piperidin-4-yl)-1-methyl-1H-indazol-3-yl)dihydro-pyrimidine-2,4(1H,3H)-dione;
1-(1-(2,2,2-trifluoroethyl)-6-(1-((1-(3-((4-((5-(trifluoromethyl)pyrimidin-2-yl)amino)piperidin-1-yl)sulfonyl)phenyl)-piperidin-4-yl)methyl)piperidin-4-yl)-1H-indazol-3-yl)dihydropyrimidine-2,4(1H,3H)-dione;
3-(1-oxo-5-(4-(3-(3-((4-((5-(trifluoro-methyl)pyrimidin-2-yl)amino)piperidin-1-yl)sulfonyl)phenoxy)azetidin-1-yl)piperidin-1-yl)isoindolin-2-yl)piperidine-2,6-dione;
1-(6-(1-(3-((4-((5-(difluoromethoxy)-pyrimidin-2-yl)amino)piperidin-1-yl)sulfonyl)benzyl)piperidin-4-yl)-7-fluoro-1-methyl-1H-indazol-3-yl)dihydropyrimidine-2,4(1H,3H)-dione;
1-(7-fluoro-1-methyl-6-(1-((1-(3-((4-((5-(trifluoromethyl)pyrimidin-2-yl)amino)-piperidin-1-yl)sulfonyl)phenyl)piperidin-4-yl)methyl)piperidin-4-yl)-1H-indazol-3-yl)dihydropyrimidine-2,4(1H,3H)-dione;
1-(6-(1-((1-(3-((4-((5-chloropyrimidin-2-yl)amino)piperidin-1-yl)sulfonyl)phenyl)-piperidin-4-yl)methyl)piperidin-4-yl)-7-fluoro-1-methyl-1H-indazol-3-yl)dihydropyrimidine-2,4(1H,3H)-dione;
3-(6-fluoro-5-(4-(2-methyl-3-(3-((4-((5-(trifluoromethyl)pyrimidin-2-yl)amino)-piperidin-1-yl)sulfonyl)phenyl)propyl)-piperazin-1-yl)-1-oxoisoindolin-2-yl)piperidine-2,6-dione;
3-(5-(4-(3-(3-((4-((5-chloropyrimidin-2-yl)amino)piperidin-1-yl)sulfonyl)phenyl)-2-methylpropyl)piperazin-1-yl)-6-fluoro-1-oxoisoindolin-2-yl)piperidine-2,6-dione;
3-(5-(4-((1-(3-((4-((5-chloropyrimidin-2-yl)amino)piperidin-1-yl)sulfonyl)phenyl)-piperidin-4-yl)methyl)piperazin-1-yl)-1-oxoisoindolin-2-yl)piperidine-2,6-dione;
1-(6-(1-((1-(3-((4-((5-chloropyrimidin-2-yl)amino)piperidin-1-yl)sulfonyl)phenyl)-piperidin-4-yl)methyl)piperidin-4-yl)-5-fluoro-1-methyl-1H-indazol-3-yl)dihydropyrimidine-2,4(1H,3H)-dione;
1-(5-fluoro-1-methyl-6-(1-((1-(3-((4-((5-(trifluoromethyl)pyrimidin-2-yl)amino)-piperidin-1-yl)sulfonyl)phenyl)piperidin-4-yl)methyl)piperidin-4-yl)-1H-indazol-3-yl)dihydropyrimidine-2,4(1H,3H)-dione;
3-(6-fluoro-1-oxo-5-(4-((1-(3-((4-((5-(trifluoromethyl)pyrimidin-2-yl)amino)-piperidin-1-yl)sulfonyl)phenyl)piperidin-4-yl)methyl)piperazin-1-yl)isoindolin-2-yl)piperidine-2,6-dione;
3-(5-(4-(3-(3-((4-((5-(difluoromethoxy)-pyrimidin-2-yl)amino)piperidin-1-yl)sulfonyl)phenyl)-2-methylpropyl)-piperazin-1-yl)-6-fluoro-1-oxoisoindolin-2-yl)piperidine-2,6-dione;
3-(5-(4-((1-(3-((4-((5-chloropyrimidin-2-yl)amino)piperidin-1-yl)sulfonyl)phenyl)-piperidin-4-yl)methyl)piperazin-1-yl)-6-fluoro-1-oxoisoindolin-2-yl)piperidine-2,6-dione;
1-(6-(3-((1-(3-((4-((5-chloropyrimidin-2-yl)amino)piperidin-1-yl)sulfonyl)phenyl)-piperidin-4-yl)methyl)-3,8-diaza-bicyclo[3.2.1]octan-8-yl)-1-methyl-1H-indazol-3-yl)dihydropyrimidine-2,4(1H,3H)-dione;
3-(5-(4-(3-(3-((4-((5-chloropyrimidin-2-yl)amino)piperidin-1-yl)sulfonyl)phenoxy)-azetidin-1-yl)piperidin-1-yl)-1-oxoisoindolin-2-yl)piperidine-2,6-dione;
3-(5-(4-((1-(3-((4-((5-(difluoromethoxy)-pyrimidin-2-yl)amino)piperidin-1-yl)sulfonyl)phenyl)piperidin-4-yl)methyl)-piperazin-1-yl)-6-fluoro-1-oxoisoindolin-2-yl)piperidine-2,6-dione;
3-(3-methyl-2-oxo-4-(1-((1-(3-((4-((5-(trifluoromethyl)pyrimidin-2-yl)amino)-piperidin-1-yl)sulfonyl)phenyl)piperidin-4-yl)methyl)piperidin-4-yl)-2,3-dihydro-1H-benzo[d]imidazol-1-yl)piperidine-2,6-dione;
1-(1-methyl-6-(1-((1-(3-((4-((5-(trifluoromethyl)pyrimidin-2-yl)amino)-piperidin-1-yl)sulfonyl)phenyl)azetidin-3-yl)methyl)piperidin-4-yl)-1H-indazol-3-yl)dihydropyrimidine-2,4(1H,3H)-dione;
1-(4-(4-((1-(3-((4-((5-chloropyrimidin-2-yl)amino)piperidin-1-yl)sulfonyl)phenyl)-piperidin-4-yl)methyl)piperazin-1-yl)phenyl)dihydropyrimidine-2,4(1H,3H)-dione;
1-(5-fluoro-1-methyl-6-(4-((1-(3-((4-((5-(trifluoromethyl)pyrimidin-2-yl)amino)-piperidin-1-yl)sulfonyl)phenyl)piperidin-4-yl)methyl)piperazin-1-yl)-1H-indazol-3-yl)dihydropyrimidine-2,4(1H,3H)-dione;
1-(6-(4-((1-(3-((4-((5-chloropyrimidin-2-yl)amino)piperidin-1-yl)sulfonyl)phenyl)-piperidin-4-yl)methyl)piperazin-1-yl)-5-fluoro-1-methyl-1H-indazol-3-yl)dihydropyrimidine-2,4(1H,3H)-dione;
1-(6-(1-((1-(3-((4-((5-(difluoromethoxy)-pyrimidin-2-yl)amino)piperidin-1-yl)sulfonyl)phenyl)piperidin-4-yl)methyl)piperidin-4-yl)-1-methyl-1H-indazol-3-yl)dihydropyrimidine-2,4(1H,3H)-dione;
1-(4-(4-((1-(3-((4-((5-(trifluoromethyl)-pyrimidin-2-yl)amino)piperidin-1-yl)sulfonyl)phenyl)piperidin-4-yl)methyl)piperazin-1-yl)phenyl)-dihydropyrimidine-2,4(1H,3H)-dione;
1-(6-(8-((1-(3-((4-((5-chloropyrimidin-2-yl)amino)piperidin-1-yl)sulfonyl)phenyl)-piperidin-4-yl)methyl)-3,8-diaza-bicyclo[3.2.1]octan-3-yl)-1-methyl-1H-indazol-3-yl)dihydropyrimidine-2,4(1H,3H)-dione;
1-(1-methyl-6-(1-((1-(3-((4-((5-(trifluoro-methoxy)pyrimidin-2-yl)amino)piperidin-1-yl)sulfonyl)phenyl)piperidin-4-yl)methyl)piperidin-4-yl)-1H-indazol-3-yl)dihydropyrimidine-2,4(1H,3H)-dione;
(R)-1-(6-(4-((1-(3-((4-((5-chloropyrimidin-2-yl)amino)piperidin-1-yl)sulfonyl)phenyl)-piperidin-4-yl)methyl)-2-methylpiperazin-1-yl)-1-methyl-1H-indazol-3-yl)dihydropyrimidine-2,4(1H,3H)-dione;
(S)-1-(6-(4-((1-(3-((4-((5-chloropyrimidin-2-yl)amino)piperidin-1-yl)sulfonyl)phenyl)-piperidin-4-yl)methyl)-2-methylpiperazin-1-yl)-1-methyl-1H-indazol-3-yl)dihydro-pyrimidine-2,4(1H,3H)-dione;
1-(1-methyl-6-(1-((1-(3-((4-((5-(trifluoro-methyl)pyrimidin-2-yl)amino)piperidin-1-yl)sulfonyl)phenyl)piperidin-4-yl)methyl)-azetidin-3-yl)-1H-indazol-3-yl)dihydropyrimidine-2,4(1H,3H)-dione;
3-(6-fluoro-1-oxo-5-(1-((1-(3-((4-((5-(trifluoromethyl)pyrimidin-2-yl)amino)-piperidin-1-yl)sulfonyl)phenyl)piperidin-4-yl)methyl)piperidin-4-yl)isoindolin-2-yl)piperidine-2,6-dione;
3-(5-(1-((1-(3-((4-((5-chloropyrimidin-2-yl)amino)piperidin-1-yl)sulfonyl)phenyl)-piperidin-4-yl)methyl)piperidin-4-yl)-6-fluoro-1-oxoisoindolin-2-yl)piperidine-2,6-dione;
3-(4-(1-((1-(3-((4-((5-(difluoromethoxy)-pyrimidin-2-yl)amino)piperidin-1-yl)sulfonyl)phenyl)piperidin-4-yl)methyl)-piperidin-4-yl)-3-methyl-2-oxo-2,3-dihydro-1H-benzo[d]imidazol-1-yl)piperidine-2,6-dione;
1-(6-(1-((3′-((4-((5-chloropyrimidin-2-yl)amino)piperidin-1-yl)sulfonyl)-[1,1′-biphenyl]-4-yl)methyl)piperidin-4-yl)-1-methyl-1H-indazol-3-yl)dihydropyrimidine-2,4(1H,3H)-dione;
1-(1-methyl-6-(1-(2-(3-((4-((5-(trifluoro-methyl)pyrimidin-2-yl)amino)piperidin-1-yl)sulfonyl)phenoxy)ethyl)piperidin-4-yl)-1H-indazol-3-yl)dihydropyrimidine-2,4(1H,3H)-dione;
1-(6-(1-(2-(3-((4-((5-chloropyrimidin-2-yl)amino)piperidin-1-yl)sulfonyl)phenoxy)-ethyl)piperidin-4-yl)-1-methyl-1H-indazol-3-yl)dihydropyrimidine-2,4(1H,3H)-dione;
1-(6-(6-((1-(3-((4-((5-chloropyrimidin-2-yl)amino)piperidin-1-yl)sulfonyl)phenyl)-piperidin-4-yl)methyl)-3,6-diazabicyclo-[3.1.1]heptan-3-yl)-1-methyl-1H-indazol-3-yl)dihydropyrimidine-2,4(1H,3H)-dione;
1-(1-methyl-6-(6-((1-(3-((4-((5-(trifluoro-methyl)pyrimidin-2-yl)amino)piperidin-1-yl)sulfonyl)phenyl)piperidin-4-yl)methyl)-3,6-diazabicyclo[3.1.1]heptan-3-yl)-1H-indazol-3-yl)dihydropyrimidine-2,4(1H,3H)-dione;
1-(6-((2S,6R)-4-((1-(3-((4-((5-chloro-pyrimidin-2-yl)amino)piperidin-1-yl)-sulfonyl)phenyl)piperidin-4-yl)methyl)-2,6-dimethylpiperazin-1-yl)-1-methyl-1H-indazol-3-yl)dihydropyrimidine-2,4(1H,3H)-dione;
3-(4-(1-(3-((4-((5-(difluoromethoxy)-pyrimidin-2-yl)amino)piperidin-1-yl)sulfonyl)benzyl)piperidin-4-yl)-3-methyl-2-oxo-2,3-dihydro-1H-benzo[d]imidazol-1-yl)piperidine-2,6-dione;
1-(6-(1-((1-(3-((4-((5-chloropyrimidin-2-yl)amino)piperidin-1-yl)sulfonyl)phenyl)-azetidin-3-yl)methyl)piperidin-4-yl)-1-methyl-1H-indazol-3-yl)dihydropyrimidine-2,4(1H,3H)-dione;
1-(1-methyl-6-(1-((5-((4-((5-(trifluoro-methyl)pyrimidin-2-yl)amino)piperidin-1-yl)sulfonyl)pyridin-3-yl)methyl)piperidin-4-yl)-1H-indazol-3-yl)dihydropyrimidine-2,4(1H,3H)-dione;
1-(6-(1-((1-(3-((4-((5-chloropyrimidin-2-yl)amino)piperidin-1-yl)sulfonyl)phenyl)-1H-pyrazol-4-yl)methyl)piperidin-4-yl)-1-methyl-1H-indazol-3-yl)dihydropyrimidine-2,4(1H,3H)-dione;
1-(6-(1-((2-(3-((4-((5-chloropyrimidin-2-yl)amino)piperidin-1-yl)sulfonyl)phenyl)-2-azaspiro[3.3]heptan-6-yl)methyl)piperidin-4-yl)-1-methyl-1H-indazol-3-yl)dihydropyrimidine-2,4(1H,3H)-dione;
3-((4-(4-((1-(3-((4-((5-(trifluoromethyl)-pyrimidin-2-yl)amino)piperidin-1-yl)sulfonyl)phenyl)piperidin-4-yl)methyl)piperazin-1-yl)phenyl)amino)-piperidine-2,6-dione;
1-(6-(1-((1-(3-((4-((5-chloropyrimidin-2-yl)amino)piperidin-1-yl)sulfonyl)phenyl)-piperidin-4-yl)methyl)-1,2,3,6-tetrahydropyridin-4-yl)-1-methyl-1H-indazol-3-yl)dihydropyrimidine-2,4(1H,3H)-dione;
(S)-1-(6-(1-((1-(3-((4-((5-chloropyrimidin-2-yl)amino)piperidin-1-yl)sulfonyl)phenyl)-pyrrolidin-3-yl)methyl)piperidin-4-yl)-1-methyl-1H-indazol-3-yl)dihydropyrimidine-2,4(1H,3H)-dione;
1-(6-(1-((1-(3-((4-((5-chloropyrimidin-2-yl)amino)piperidin-1-yl)sulfonyl)phenyl)-azepan-4-yl)methyl)piperidin-4-yl)-1-methyl-1H-indazol-3-yl)dihydropyrimidine-2,4(1H,3H)-dione;
1-(1-methyl-5-(1-((1-(3-((4-((5-(trifluoro-methyl)pyrimidin-2-yl)amino)piperidin-1-yl)sulfonyl)phenyl)piperidin-4-yl)methyl)-piperidin-4-yl)-1H-indazol-3-yl)dihydropyrimidine-2,4(1H,3H)-dione;
1-(6-(4-((4-(3-((4-((5-chloropyrimidin-2-yl)amino)piperidin-1-yl)sulfonyl)phenyl)-piperazin-1-yl)methyl)piperidin-1-yl)-1-methyl-1H-indazol-3-yl)dihydropyrimidine-2,4(1H,3H)-dione;
1-(6-(1-(4-(1-((4-((5-chloropyrimidin-2-yl)amino)piperidin-1-yl)sulfonyl)piperidin-4-yl)benzyl)piperidin-4-yl)-1-methyl-1H-indazol-3-yl)dihydropyrimidine-2,4(1H,3H)-dione;
1-(6-(1-((1-(5-((4-((5-chloropyrimidin-2-yl)amino)piperidin-1-yl)sulfonyl)-2-fluorophenyl)piperidin-4-yl)methyl)-piperidin-4-yl)-1-methyl-1H-indazol-3-yl)dihydropyrimidine-2,4(1H,3H)-dione;
ethyl 1-(3-((4-((5-chloropyrimidin-2-yl)amino)piperidin-1-yl)sulfonyl)phenyl)-4-((4-(3-(2,4-dioxotetrahydropyrimidin-1(2H)-yl)-1-methyl-1H-indazol-6-yl)piperidin-1-yl)methyl)piperidine-4-carboxylate;
1-(6-(1-((1-(2-fluoro-5-((4-((5-(trifluoro-methyl)pyrimidin-2-yl)amino)piperidin-1-yl)sulfonyl)phenyl)piperidin-4-yl)methyl)-piperidin-4-yl)-1-methyl-1H-indazol-3-yl)dihydropyrimidine-2,4(1H,3H)-dione;
1-(6-(5-((1-(3-((4-((5-chloropyrimidin-2-yl)amino)piperidin-1-yl)sulfonyl)phenyl)-piperidin-4-yl)methyl)-2,5-diazabicyclo-[4.1.0]heptan-2-yl)-1-methyl-1H-indazol-3-yl)dihydropyrimidine-2,4(1H,3H)-dione;
(R)-1-(6-(1-((1-(3-((4-((5-chloropyrimidin-2-yl)amino)piperidin-1-yl)sulfonyl)phenyl)-pyrrolidin-3-yl)methyl)piperidin-4-yl)-1-methyl-1H-indazol-3-yl)dihydropyrimidine-2,4(1H,3H)-dione;
1-(1-methyl-7-(1-((1-(3-((4-((5-(trifluoro-methyl)pyrimidin-2-yl)amino)piperidin-1-yl)sulfonyl)phenyl)piperidin-4-yl)methyl)-piperidin-4-yl)-1H-indazol-3-yl)dihydropyrimidine-2,4(1H,3H)-dione;
1-(1-methyl-6-(1-(3-((6-((5-(trifluoro-methyl)pyrimidin-2-yl)amino)-2-azaspiro[3.3]-heptan-2-yl)sulfonyl)benzyl)-piperidin-4-yl)-1H-indazol-3-yl)dihydropyrimidine-2,4(1H,3H)-dione;
1-(6-(1-((1-(3-((4-((5-chloropyrimidin-2-yl)amino)piperidin-1-yl)sulfonyl)-5-fluorophenyl)piperidin-4-yl)methyl)piperidin-4-yl)-1-methyl-1H-indazol-3-yl)dihydro-pyrimidine-2,4(1H,3H)-dione;
4-((4-((5-chloropyrimidin-2-yl)amino)piperidin-1-yl)sulfonyl)-2-(4-((4-(3-(2,4-dioxotetrahydropyrimidin-1(2H)-yl)-1-methyl-1H-indazol-6-yl)piperidin-1-yl)methyl)-piperidin-1-yl)benzonitrile;
1-(6-(1-((1-(3-fluoro-5-((4-((5-(trifluoromethyl)pyrimidin-2-yl)amino)piperidin-1-yl)sulfonyl)phenyl)piperidin-4-yl)methyl)piperidin-4-yl)-1-methyl-1H-indazol-3-yl)dihydropyrimidine-2,4(1H,3H)-dione;
4-((4-((5-chloropyrimidin-2-yl)amino)piperidin-1-yl)sulfonyl)-2-(4-((4-(3-(2,4-dioxotetrahydropyrimidin-1(2H)-yl)-7-fluoro-1-methyl-1H-indazol-6-yl)piperidin-1-yl)methyl)piperidin-1-yl)benzonitrile;
4-((4-((5-chloropyrimidin-2-yl)amino)piperidin-1-yl)sulfonyl)-2-(4-((4-(1-(2,6-dioxopiperidin-3-yl)-3-methyl-2-oxo-2,3-dihydro-1H-benzo[d]imidazol-4-yl)piperidin-1-yl)methyl)piperidin-1-yl)benzonitrile;
2-(4-((4-(3-(2,4-dioxotetrahydropyrimidin-1(2H)-yl)-1-methyl-1H-indazol-6-yl)piperidin-1-yl)methyl)piperidin-1-yl)-4-((4-((5-(trifluoromethyl)pyrimidin-2-yl)amino)-piperidin-1-yl)sulfonyl)benzonitrile;
4-((4-((5-chloropyrimidin-2-yl)amino)piperidin-1-yl)sulfonyl)-2-(4-((4-(3-(2,4-dioxotetrahydropyrimidin-1(2H)-yl)-5-fluoro-1-methyl-1H-indazol-6-yl)piperidin-1-yl)methyl)piperidin-1-yl)benzonitrile;
1-(1-methyl-6-(1-(3-((3-((5-(trifluoromethyl)pyrimidin-2-yl)amino)azetidin-1-yl)sulfonyl)benzyl)piperidin-4-yl)-1H-indazol-3-yl)dihydropyrimidine-2,4(1H,3H)-dione;
1-(1-methyl-6-(1-((1-(3-((4-((5-(1-methyl-1H-pyrazol-4-yl)pyrimidin-2-yl)amino)piperidin-1-yl)sulfonyl)phenyl)piperidin-4-yl)methyl)piperidin-4-yl)-1H-indazol-3-yl)dihydropyrimidine-2,4(1H,3H)-dione;
1-(6-(1-((4-(3-((4-((5-chloropyrimidin-2-yl)amino)piperidin-1-yl)sulfonyl)phenyl)-cyclohexyl)methyl)piperidin-4-yl)-1-methyl-1H-indazol-3-yl)dihydropyrimidine-2,4(1H,3H)-dione 2,2,2-trifluoroacetate;
1-(6-(1-(4-(1-((4-((5-chloropyrimidin-2-yl)amino)piperidin-1-yl)sulfonyl)piperidin-3-yl)benzyl)piperidin-4-yl)-1-methyl-1H-indazol-3-yl)dihydropyrimidine-2,4(1H,3H)-dione;
1-(6-(1-((1-(4-((4-((5-chloropyrimidin-2-yl)amino)piperidin-1-yl)sulfonyl)phenyl)-piperidin-4-yl)methyl)piperidin-4-yl)-1-methyl-1H-indazol-3-yl)dihydropyrimidine-2,4(1H,3H)-dione;
1-(6-(4-((1-(3-((4-((5-chloropyrimidin-2-yl)amino)piperidin-1-yl)sulfonyl)phenyl)-piperidin-4-yl)methyl)-2,2-dimethylpiperazin-1-yl)-1-methyl-1H-indazol-3-yl)dihydropyrimidine-2,4(1H,3H)-dione;
3-((4-((5-chloropyrimidin-2-yl)amino)piperidin-1-yl)sulfonyl)-5-(4-((4-(3-(2,4-dioxotetrahydropyrimidin-1(2H)-yl)-1-methyl-1H-indazol-6-yl)piperidin-1-yl)methyl)-piperidin-1-yl)benzonitrile 2,2,2-trifluoroacetate;
2-((4-((5-chloropyrimidin-2-yl)amino)piperidin-1-yl)sulfonyl)-4-(4-((4-(3-(2,4-dioxotetrahydropyrimidin-1(2H)-yl)-1-methyl-1H-indazol-6-yl)piperidin-1-yl)methyl)-piperidin-1-yl)benzonitrile;
1-(6-(1-((1-(3-((4-((5-chloropyrimidin-2-yl)amino)piperidin-1-yl)sulfonyl)phenyl)-4-hydroxypiperidin-4-yl)methyl)piperidin-4-yl)-1-methyl-1H-indazol-3-yl)dihydropyrimidine-2,4(1H,3H)-dione;
1-(6-(1-((1-(1-((4-((5-chloropyrimidin-2-yl)amino)piperidin-1-yl)sulfonyl)piperidin-4-yl)-1H-pyrazol-4-yl)methyl)piperidin-4-yl)-1-methyl-1H-indazol-3-yl)dihydropyrimidine-2,4(1H,3H)-dione;
1-(3-((4-((5-chloropyrimidin-2-yl)amino)piperidin-1-yl)sulfonyl)phenyl)-4-((4-(3-(2,4-dioxotetrahydropyrimidin-1(2H)-yl)-1-methyl-1H-indazol-6-yl)piperidin-1-yl)methyl)-piperidine-4-carbonitrile;
1-(1-methyl-6-(1-((1-(4-((4-((5-(trifluoromethyl)pyrimidin-2-yl)amino)piperidin-1-yl)sulfonyl)phenyl)piperidin-4-yl)methyl)piperidin-4-yl)-1H-indazol-3-yl)dihydropyrimidine-2,4(1H,3H)-dione;
1-(6-(4-((3-(3-((4-((5-chloropyrimidin-2-yl)amino)piperidin-1-yl)sulfonyl)phenyl)-3,8-diazabicyclo[3.2.1]octan-8-yl)methyl)piperidin-1-yl)-1-methyl-1H-indazol-3-yl)dihydro-pyrimidine-2,4(1H,3H)-dione;
3-(3-methyl-2-oxo-6-(1-((1-(3-((4-((5-(trifluoromethyl)pyrimidin-2-yl)amino)-piperidin-1-yl)sulfonyl)phenyl)piperidin-4-yl)methyl)piperidin-4-yl)-2,3-dihydro-1H-benzo[d]imidazol-1-yl)piperidine-2,6-dione;
3-(7-(1-((1-(3-((4-((5-chloropyrimidin-2-yl)amino)piperidin-1-yl)sulfonyl)phenyl)-piperidin-4-yl)methyl)piperidin-4-yl)-2-oxobenzo[d]oxazol-3 (2H)-yl)piperidine-2,6-dione;
4-((4-((5-(difluoromethoxy)pyrimidin-2-yl)amino)piperidin-1-yl)sulfonyl)-2-((4-(3-(2,4-dioxotetrahydropyrimidin-1(2H)-yl)-1-methyl-1H-indazol-6-yl)piperidin-1-yl)methyl)benzonitrile;
1-(6-(1-((1-(3-((4-((5-chloropyrimidin-2-yl)amino)piperidin-1-yl)sulfonyl)-5-(trifluoromethyl)phenyl)piperidin-4-yl)methyl)piperidin-4-yl)-1-methyl-1H-indazol-3-yl)dihydropyrimidine-2,4(1H,3H)-dione;
3-(2-oxo-3-(1-((1-(3-((4-((5-(trifluoromethyl)pyrimidin-2-yl)amino)piperidin-1-yl)sulfonyl)phenyl)piperidin-4-yl)methyl)piperidin-4-yl)-2,3-dihydro-1H-benzo[d]imidazol-1-yl)piperidine-2,6-dione;
1-(1-methyl-6-(1-((1-(3-((6-((5-(trifluoromethyl)pyrimidin-2-yl)amino)-2-azaspiro[3.3]heptan-2-yl)sulfonyl)phenyl)piperidin-4-yl)methyl)piperidin-4-yl)-1H-indazol-3-yl)dihydropyrimidine-2,4(1H,3H)-dione;
3-(4-(1-((1-(3-((4-((5-chloropyrimidin-2-yl)amino)piperidin-1-yl)sulfonyl)-5-(trifluoromethyl)phenyl)piperidin-4-yl)methyl)piperidin-4-yl)-3-methyl-2-oxo-2,3-dihydro-1H-benzo[d]imidazol-1-yl)piperidine-2,6-dione;
3-(4-(1-((1-(3-((4-((5-(difluoromethoxy)pyrimidin-2-yl)amino)piperidin-1-yl)sulfonyl)-5-fluorophenyl)piperidin-4-yl)methyl)piperidin-4-yl)-3-methyl-2-oxo-2,3-dihydro-1H-benzo[d]imidazol-1-yl)piperidine-2,6-dione;
1-(6-(1-((1-(3-((4-((5-(difluoromethoxy)pyrimidin-2-yl)amino)piperidin-1-yl)sulfonyl)-5-fluorophenyl)piperidin-4-yl)methyl)piperidin-4-yl)-1-methyl-1H-indazol-3-yl)dihydro-pyrimidine-2,4(1H,3H)-dione;
3-(4-(1-((1-(3-((4-((5-(difluoromethoxy)pyrimidin-2-yl)amino)piperidin-1-yl)sulfonyl)-5-(trifluoromethyl)phenyl)piperidin-4-yl)methyl)piperidin-4-yl)-3-methyl-2-oxo-2,3-dihydro-1H-benzo[d]imidazol-1-yl)piperidine-2,6-dione;
3-(3-methyl-2-oxo-5-(1-((1-(3-((4-((5-(trifluoromethyl)pyrimidin-2-yl)amino)-piperidin-1-yl)sulfonyl)phenyl)piperidin-4-yl)methyl)piperidin-4-yl)-2,3-dihydro-1H-benzo[d]imidazol-1-yl)piperidine-2,6-dione;
1-(1-methyl-6-(1-((1-(3-((3-((5-(trifluoromethyl)pyrimidin-2-yl)amino)azetidin-1-yl)sulfonyl)phenyl)piperidin-4-yl)methyl)piperidin-4-yl)-1H-indazol-3-yl)dihydropyrimidine-2,4(1H,3H)-dione;
1-(1-methyl-6-((4-(3-((4-((5-(trifluoromethyl)pyrimidin-2-yl)amino)piperidin-1-yl)sulfonyl)phenyl)piperazin-1-yl)methyl)-1H-indazol-3-yl)dihydropyrimidine-2,4(1H,3H)-dione;
3-(4-(1-((1-(3-((4-((5-chloropyrimidin-2-yl)amino)piperidin-1-yl)sulfonyl)-5-fluorophenyl)piperidin-4-yl)methyl)piperidin-4-yl)-3-methyl-2-oxo-2,3-dihydro-1H-benzo[d]imidazol-1-yl)piperidine-2,6-dione;
2-(4-((4-(1-(2,6-dioxopiperidin-3-yl)-3-methyl-2-oxo-2,3-dihydro-1H-benzo[d]-imidazol-4-yl)piperidin-1-yl)methyl)piperidin-1-yl)-4-((4-((5-(trifluoromethyl)pyrimidin-2-yl)amino)piperidin-1-yl)sulfonyl)benzonitrile;
1-(3-(1-((1-(3-((4-((5-chloropyrimidin-2-yl)amino)piperidin-1-yl)sulfonyl)phenyl)-piperidin-4-yl)methyl)piperidin-4-yl)phenyl)dihydropyrimidine-2,4(1H,3H)-dione;
1-(1-methyl-6-(1-((2-((4-((5-(trifluoromethyl)pyrimidin-2-yl)amino)piperidin-1-yl)sulfonyl)pyridin-4-yl)methyl)piperidin-4-yl)-1H-indazol-3-yl)dihydropyrimidine-2,4(1H,3H)-dione;
3-(4-(1-(3-((4-((5-(difluoromethoxy)pyrimidin-2-yl)amino)piperidin-1-yl)sulfonyl)-5-fluorobenzyl)piperidin-4-yl)-3-methyl-2-oxo-2,3-dihydro-1H-benzo[d]imidazol-1-yl)piperidine-2,6-dione;
4-((4-((5-chloropyrimidin-2-yl)amino)piperidin-1-yl)sulfonyl)-2-(4-((4-(2-(2,6-dioxopiperidin-3-yl)-6-fluoro-1,3-dioxoisoindolin-5-yl)piperazin-1-yl)methyl)piperidin-1-yl)benzonitrile;
3-(2-oxo-3-((1-((1-(3-((4-((5-(trifluoromethyl)pyrimidin-2-yl)amino)piperidin-1-yl)sulfonyl)phenyl)piperidin-4-yl)methyl)piperidin-4-yl)methyl)-2,3-dihydro-1H-benzo[d]imidazol-1-yl)piperidine-2,6-dione;
1-(6-(1-((1-(3-((4-((5-chloropyrimidin-2-yl)amino)piperidin-1-yl)sulfonyl)-2-fluorophenyl)piperidin-4-yl)methyl)piperidin-4-yl)-1-methyl-1H-indazol-3-yl)dihydropyrimidine-2,4(1H,3H)-dione;
2-(4-((4-(3-(2,4-dioxotetrahydropyrimidin-1(2H)-yl)-7-fluoro-1-methyl-1H-indazol-6-yl)piperidin-1-yl)methyl)piperidin-1-yl)-4-((4-((5-(trifluoromethyl)pyrimidin-2-yl)amino)piperidin-1-yl)sulfonyl)benzonitrile;
1-(6-(1-((1-(3-((4-((5-chloropyrimidin-2-yl)amino)piperidin-1-yl)sulfonyl)-4-fluorophenyl)piperidin-4-yl)methyl)piperidin-4-yl)-1-methyl-1H-indazol-3-yl)dihydro-pyrimidine-2,4(1H,3H)-dione;
3-(3-methyl-4-(1-(3-(methyl (3-((4-((5-(trifluoromethyl)pyrimidin-2-yl)amino)-piperidin-1-yl)sulfonyl)phenyl)amino)propyl)piperidin-4-yl)-2-oxo-2,3-dihydro-1H-benzo[d]imidazol-1-yl)piperidine-2,6-dione;
1-(6-(4-((8-(3-((4-((5-chloropyrimidin-2-yl)amino)piperidin-1-yl)sulfonyl)phenyl)-3,8-diazabicyclo[3.2.1]octan-3-yl)methyl)piperidin-1-yl)-1-methyl-1H-indazol-3-yl)dihydro-pyrimidine-2,4(1H,3H)-dione;
4-((4-((5-(difluoromethoxy)pyrimidin-2-yl)amino)piperidin-1-yl)sulfonyl)-2-(4-((4-(3-(2,4-dioxotetrahydropyrimidin-1(2H)-yl)-7-fluoro-1-methyl-1H-indazol-6-yl)piperidin-1-yl)methyl)piperidin-1-yl)benzonitrile;
1-(6-(1-((1-(3-((4-((5-fluoropyrimidin-2-yl)amino)piperidin-1-yl)sulfonyl)phenyl)-piperidin-4-yl)methyl)piperidin-4-yl)-1-methyl-1H-indazol-3-yl)dihydropyrimidine-2,4(1H,3H)-dione;
1-(8-(1-((1-(3-((4-((5-chloropyrimidin-2-yl)amino)piperidin-1-yl)sulfonyl)phenyl)-piperidin-4-yl)methyl)piperidin-4-yl)isoquinolin-4-yl)dihydropyrimidine-2,4(1H,3H)-dione;
1-(6-(1-((1-(3-((4-((5-(difluoromethyl)pyrimidin-2-yl)amino)piperidin-1-yl)sulfonyl)phenyl)piperidin-4-yl)methyl)piperidin-4-yl)-1-methyl-1H-indazol-3-yl)dihydropyrimidine-2,4(1H,3H)-dione;
4-((4-((5-(difluoromethoxy)pyrimidin-2-yl)amino)piperidin-1-yl)sulfonyl)-2-((4-(1-(2,6-dioxopiperidin-3-yl)-3-methyl-2-oxo-2,3-dihydro-1H-benzo[d]imidazol-4-yl)piperidin-1-yl)methyl)benzonitrile;
4-((4-((5-chloropyrimidin-2-yl)amino)piperidin-1-yl)sulfonyl)-2-(4-((4-(3-(2,6-dioxopiperidin-3-yl)-2-oxo-2,3-dihydrobenzo[d]oxazol-7-yl)piperidin-1-yl)methyl)piperidin-1-yl)benzonitrile;
2-(4-((4-(3-(2,6-dioxopiperidin-3-yl)-2-oxo-2,3-dihydrobenzo[d]oxazol-7-yl)piperidin-1-yl)methyl)piperidin-1-yl)-4-((4-((5-(trifluoromethyl)pyrimidin-2-yl)amino)piperidin-1-yl)sulfonyl)benzonitrile;
1-(6-(1-((1-(3-((4-((5-bromopyrimidin-2-yl)amino)piperidin-1-yl)sulfonyl)phenyl)-piperidin-4-yl)methyl)piperidin-4-yl)-1-methyl-1H-indazol-3-yl)dihydropyrimidine-2,4(1H,3H)-dione;
4-((4-((5-chloropyrimidin-2-yl)amino)piperidin-1-yl)sulfonyl)-2-(4-((4-(2-(2,6-dioxopiperidin-3-yl)-1-oxoisoindolin-5-yl)piperazin-1-yl)methyl)piperidin-1-yl)benzonitrile;
3-(2-oxo-7-(1-((1-(3-((4-((5-(trifluoromethyl)pyrimidin-2-yl)amino)piperidin-1-yl)sulfonyl)phenyl)piperidin-4-yl)methyl)piperidin-4-yl)benzo[d]oxazol-3 (2H)-yl)piperidine-2,6-dione;
Rac-1-(6-(1-((1-(3-(((3R,4S)-4-((5-chloropyrimidin-2-yl)amino)-3-fluoropiperidin-1-yl)sulfonyl)phenyl)piperidin-4-yl)methyl)piperidin-4-yl)-1-methyl-1H-indazol-3-yl)dihydropyrimidine-2,4(1H,3H)-dione;
4-((4-((5-chloropyrimidin-2-yl)amino)piperidin-1-yl)sulfonyl)-2-(4-((4-(1-(2,6-dioxopiperidin-3-yl)-3-methyl-2-oxo-2,3-dihydro-1H-benzo[d]imidazol-4-yl)piperazin-1-yl)methyl)piperidin-1-yl)benzonitrile;
4-((4-((5-chloropyrimidin-2-yl)amino)piperidin-1-yl)sulfonyl)-2-(4-((4-(1-(2,6-dioxopiperidin-3-yl)-3-methyl-2-oxo-2,3-dihydro-1H-benzo[d]imidazol-5-yl)piperidin-1-yl)methyl)piperidin-1-yl)benzonitrile;
2-(4-((4-(1-(2,6-dioxopiperidin-3-yl)-3-methyl-2-oxo-2,3-dihydro-1H-benzo[d]imidazol-5-yl)piperidin-1-yl)methyl)piperidin-1-yl)-4-((4-((5-(trifluoromethyl)-pyrimidin-2-yl)amino)piperidin-1-yl)sulfonyl)benzonitrile;
1-(6-(1-((1-(3-((4-((5-chloropyrimidin-2-yl)amino)piperidin-1-yl)sulfonyl)phenyl)-4-methylpiperidin-4-yl)methyl)piperidin-4-yl)-1-methyl-1H-indazol-3-yl)dihydropyrimidine-2,4(1H,3H)-dione;
4-((4-((5-chloropyrimidin-2-yl)amino)piperidin-1-yl)sulfonyl)-2-(4-((4-(1-(2,6-dioxopiperidin-3-yl)-3,3-dimethyl-2-oxoindolin-4-yl)piperidin-1-yl)methyl)piperidin-1-yl)benzonitrile;
1-(1-methyl-6-(1-((1-(3-((4-((5-vinylpyrimidin-2-yl)amino)piperidin-1-yl)sulfonyl)-phenyl)piperidin-4-yl)methyl)piperidin-4-yl)-1H-indazol-3-yl)dihydropyrimidine-2,4(1H,3H)-dione;
4-((4-((5-chloropyrimidin-2-yl)amino)piperidin-1-yl)sulfonyl)-2-(4-((4-(1-(2,6-dioxopiperidin-3-yl)-6-fluoro-3-methyl-2-oxo-2,3-dihydro-1H-benzo[d]imidazol-4-yl)piperidin-1-yl)methyl)piperidin-1-yl)benzonitrile;
1-(6-(1-((1-(3-((4-((5-chloropyrimidin-2-yl)amino)piperidin-1-yl)sulfonyl)-phenyl)piperidin-4-yl)methyl)piperidin-4-yl)-1-methyl-1H-indazol-3-yl)pyrimidine-2,4(1H,3H)-dione;
1-(1-methyl-6-(1-((1-(3-((4-((5-(trifluoromethyl)pyrimidin-2-yl)amino)piperidin-1-yl)sulfonyl)phenyl)piperidin-4-yl)methyl)piperidin-4-yl)-1H-indazol-3-yl)pyrimidine-2,4(1H,3H)-dione;
1-(1-methyl-6-(1-((1-(3-((4-((5-(trifluoromethyl)pyrimidin-2-yl)amino)piperidin-1-yl)sulfonyl)phenyl)piperidin-4-yl)methyl)piperidin-4-yl)-1H-pyrazolo[4,3-b]pyridin-3-yl)dihydropyrimidine-2,4(1H,3H)-dione;
1-(8-(1-((1-(3-((4-((5-chloropyrimidin-2-yl)amino)piperidin-1-yl)sulfonyl)phenyl)-piperidin-4-yl)methyl)piperidin-4-yl)imidazo[1,2-a]pyridin-3-yl)dihydropyrimidine-2,4(1H,3H)-dione;
4-((4-((5-chloropyrimidin-2-yl)amino)piperidin-1-yl)sulfonyl)-2-(4-((4-(3-(2,4-dioxotetrahydropyrimidin-1(2H)-yl)-1-methyl-1H-indazol-6-yl)piperidin-1-yl)methyl)-4-hydroxypiperidin-1-yl)benzonitrile;
1-(6-(1-((1-(3-((4-((5-chloropyrimidin-2-yl)amino)piperidin-1-yl)sulfonyl)phenyl)-piperidin-4-yl)methyl)piperidin-4-yl)-1-methyl-1H-pyrazolo[4,3-b]pyridin-3-yl)dihydropyrimidine-2,4(1H,3H)-dione;
4-((4-((5-chloropyrimidin-2-yl)amino)piperidin-1-yl)sulfonyl)-2-(4-((4-(1-(2,6-dioxopiperidin-3-yl)-5-fluoro-3-methyl-2-oxo-2,3-dihydro-1H-benzo[d]imidazol-4-yl)piperidin-1-yl)methyl)piperidin-1-yl)benzonitrile;
2-(4-((4-(1-(2,6-dioxopiperidin-3-yl)-3-methyl-2-oxo-2,3-dihydro-1H-benzo[d]-imidazol-4-yl)piperazin-1-yl)methyl)piperidin-1-yl)-4-((4-((5-(trifluoromethyl)pyrimidin-2-yl)amino)piperidin-1-yl)sulfonyl)benzonitrile;
1-(6-(1-((1-(3-(((3R,4S)-4-((5-chloropyrimidin-2-yl)amino)-3-methoxypiperidin-1-yl)sulfonyl)phenyl)piperidin-4-yl)methyl)piperidin-4-yl)-1-methyl-1H-indazol-3-yl)dihydro-pyrimidine-2,4(1H,3H)-dione;
Rac-1-(6-(1-((1-(3-(((3R,4R)-4-((5-chloropyrimidin-2-yl)amino)-3-methoxypiperidin-1-yl)sulfonyl)phenyl)piperidin-4-yl)methyl)piperidin-4-yl)-1-methyl-1H-indazol-3-yl)dihydropyrimidine-2,4(1H,3H)-dione;
4-((4-((5-chloropyrimidin-2-yl)amino)piperidin-1-yl)sulfonyl)-2-(4-((4-(1-(2,6-dioxopiperidin-3-yl)-7-fluoro-3-methyl-2-oxo-2,3-dihydro-1H-benzo[d]imidazol-4-yl)piperidin-1-yl)methyl)piperidin-1-yl)benzonitrile;
1-(6-(1-((1-(3-((4-((5-chloropyrimidin-2-yl)amino)piperidin-1-yl)sulfonyl)phenyl)-piperidin-4-yl)methyl)piperidin-4-yl)benzo[d]isoxazol-3-yl)dihydropyrimidine-2,4(1H,3H)-dione;
1-(6-(1-((1-(3-((4-((5-(trifluoromethyl)pyrimidin-2-yl)amino)piperidin-1-yl)sulfonyl)phenyl)piperidin-4-yl)methyl)piperidin-4-yl)benzo[d]isoxazol-3-yl)dihydropyrimidine-2,4(1H,3H)-dione;
Rac-1-(6-(1-((1-(3-(((3R,4S)-3-fluoro-4-((5-(trifluoromethyl)pyrimidin-2-yl)amino)piperidin-1-yl)sulfonyl)phenyl)piperidin-4-yl)methyl)piperidin-4-yl)-1-methyl-1H-indazol-3-yl)dihydropyrimidine-2,4(1H,3H)-dione;
1-(6-(4-((4-(3-((4-((5-chloropyrimidin-2-yl)amino)piperidin-1-yl)sulfonyl)phenyl)-piperazin-1-yl)methyl)cyclohexyl)-1-methyl-1H-indazol-3-yl)dihydropyrimidine-2,4(1H,3H)-dione;
N-(2,6-dioxopiperidin-3-yl)-5-(1-((1-(3-((4-((5-(trifluoromethyl)pyrimidin-2-yl)amino)piperidin-1-yl)sulfonyl)phenyl)piperidin-4-yl)methyl)piperidin-4-yl)picolinamide;
1-(6-(1-(3-(3-((4-((5-chloropyrimidin-2-yl)amino)piperidin-1-yl)sulfonyl)phenyl)prop-2-yn-1-yl)piperidin-4-yl)-1-methyl-1H-indazol-3-yl)dihydropyrimidine-2,4(1H,3H)-dione;
1-(6-(1-((1-(3-(((1R,5S)-3-((5-chloropyrimidin-2-yl)amino)-8-azabicyclo[3.2.1]octan-8-yl)sulfonyl)phenyl)piperidin-4-yl)methyl)piperidin-4-yl)-1-methyl-1H-indazol-3-yl)dihydropyrimidine-2,4(1H,3H)-dione;
1-(6-(1-((1-(3-((4-((5-chloropyrimidin-2-yl)amino)piperidin-1-yl)sulfonyl)phenyl)-piperidin-4-yl)methyl)piperidin-4-yl)-1-methyl-1H-pyrazolo[4,3-c]pyridin-3-yl)dihydropyrimidine-2,4(1H,3H)-dione;
Rac-1-(6-(1-((1-(3-(((1R,5S,8s)-8-((5-chloropyrimidin-2-yl)amino)-3-azabicyclo-[3.2.1]octan-3-yl)sulfonyl)phenyl)piperidin-4-yl)methyl)piperidin-4-yl)-1-methyl-1H-indazol-3-yl)dihydropyrimidine-2,4(1H,3H)-dione;
1-(1-methyl-6-(3-(4-(3-((4-((5-(trifluoromethyl)pyrimidin-2-yl)amino)piperidin-1-yl)sulfonyl)phenyl)piperazin-1-yl)prop-1-yn-1-yl)-1H-indazol-3-yl)dihydropyrimidine-2,4(1H,3H)-dione;
1-(6-(3-(4-(3-((4-((5-chloropyrimidin-2-yl)amino)piperidin-1-yl)sulfonyl)-phenyl)piperazin-1-yl)prop-1-yn-1-yl)-1-methyl-1H-indazol-3-yl)dihydropyrimidine-2,4(1H,3H)-dione;
1-(1-methyl-6-(1-(4-((3-((4-((5-(trifluoromethyl)pyrimidin-2-yl)amino)piperidin-1-yl)sulfonyl)phenyl)ethynyl)benzyl)piperidin-4-yl)-1H-indazol-3-yl)dihydropyrimidine-2,4(1H,3H)-dione;
1-(6-(1-(4-((3-((4-((5-chloropyrimidin-2-yl)amino)piperidin-1-yl)sulfonyl)phenyl)-ethynyl)benzyl)piperidin-4-yl)-1-methyl-1H-indazol-3-yl)dihydropyrimidine-2,4(1H,3H)-dione;
1-(6-(1-((1-(3-((4-((5-chloropyrimidin-2-yl)amino)piperidin-1-yl)sulfonyl)phenyl)-2-oxopiperidin-4-yl)methyl)piperidin-4-yl)-1-methyl-1H-indazol-3-yl)dihydropyrimidine-2,4(1H,3H)-dione;
1-(1-methyl-6-(3-(4-(3-((4-((5-(trifluoromethyl)pyrimidin-2-yl)amino)piperidin-1-yl)sulfonyl)phenyl)piperazin-1-yl)propyl)-1H-indazol-3-yl)dihydropyrimidine-2,4(1H,3H)-dione;
N-(2,6-dioxopiperidin-3-yl)-2-fluoro-4-(1-((1-(3-((4-((5-(trifluoromethyl)pyrimidin-2-yl)amino)piperidin-1-yl)sulfonyl)phenyl)piperidin-4-yl)methyl)piperidin-4-yl)benzamide;
4-(1-((1-(3-((4-((5-chloropyrimidin-2-yl)amino)piperidin-1-yl)sulfonyl)phenyl)-piperidin-4-yl)methyl)piperidin-4-yl)-N-(2,6-dioxopiperidin-3-yl)-2-fluorobenzamide;
1-(1-methyl-6-(4-((4-(3-((4-((5-(trifluoromethyl)pyrimidin-2-yl)amino)piperidin-1-yl)sulfonyl)phenyl)piperidin-1-yl)methyl)cyclohexyl)-1H-indazol-3-yl)dihydropyrimidine-2,4(1H,3H)-dione;
1-(6-(1-((1-(3-((4-((5-chloropyrimidin-2-yl)amino)piperidin-1-yl)sulfonyl)phenyl)-piperidin-4-yl)methyl)piperidin-4-yl)-1-methyl-1H-indazol-3-yl)-4-thioxotetrahydropyrimidin-2 (1H)-one:
1-(6-(1-((1-(3-(((1r,4r)-4-((5-chloropyrimidin-2-yl)amino)cyclohexyl)sulfonyl)-phenyl)piperidin-4-yl)methyl)piperidin-4-yl)-1-methyl-1H-indazol-3-yl)dihydropyrimidine-2,4(1H,3H)-dione;
1-(6-(1-(4-((4-((5-(difluoromethoxy)pyrimidin-2-yl)amino)piperidin-1-yl)sulfonyl)-benzyl)piperidin-4-yl)-1-methyl-1H-indazol-3-yl)dihydropyrimidine-2,4(1H,3H)-dione;
1-(6-(1-(4-((4-((5-(difluoromethoxy)pyrimidin-2-yl)amino)piperidin-1-yl)sulfonyl)benzyl)piperidin-4-yl)-7-fluoro-1-methyl-1H-indazol-3-yl)dihydropyrimidine-2,4(1H,3H)-dione;
1-(1-methyl-6-(1-((1-(3-((4-((5-(2,2,2-trifluoroethyl)pyrimidin-2-yl)amino)piperidin-1-yl)sulfonyl)phenyl)piperidin-4-yl)methyl)piperidin-4-yl)-1H-indazol-3-yl)dihydropyrimidine-2,4(1H,3H)-dione;
1-(6-(1-((1-(3-((4-((5-isopropylpyrimidin-2-yl)amino)piperidin-1-yl)sulfonyl)-phenyl)piperidin-4-yl)methyl)piperidin-4-yl)-1-methyl-1H-indazol-3-yl)dihydropyrimidine-2,4(1H,3H)-dione;
1-(1-methyl-6-(1-((1-(3-((4-((5-(trifluoromethyl)pyrimidin-2-yl)amino)phenyl)-sulfonyl)phenyl)piperidin-4-yl)methyl)piperidin-4-yl)-1H-indazol-3-yl)dihydropyrimidine-2,4(1H,3H)-dione;
3-(6-(1-((1-(3-((4-((5-chloropyrimidin-2-yl)amino)piperidin-1-yl)sulfonyl)phenyl)-piperidin-4-yl)methyl)piperidin-4-yl)-1-methyl-1H-indazol-3-yl)piperidine-2,6-dione;
3-(5-(4-((4-(3-((4-((5-chloropyrimidin-2-yl)amino)piperidin-1-yl)sulfonyl)phenyl)piperazin-1-yl)methyl)piperidin-1-yl)-1-oxoisoindolin-2-yl)piperidine-2,6-dione;
3-(1-oxo-5-(4-((4-(3-((4-((5-(trifluoromethyl)pyrimidin-2-yl)amino)piperidin-1-yl)sulfonyl)phenyl)piperazin-1-yl)methyl)piperidin-1-yl)isoindolin-2-yl)piperidine-2,6-dione;
1-(6-(1-((1-(3-((4-((5-chloropyrimidin-2-yl)amino)piperidin-1-yl)sulfonyl)phenyl)piperidin-4-yl)methyl)azetidin-3-yl)-1-methyl-1H-indazol-3-yl)dihydropyrimidine-2,4(1H,3H)-dione;
3-(6-(1-((1-(3-((4-((5-(trifluoromethyl)pyrimidin-2-yl)amino)piperidin-1-yl)sulfonyl)-phenyl)piperidin-4-yl)methyl)piperidin-4-yl)benzo[b]thiophen-3-yl)piperidine-2,6-dione;
3-(1-methyl-6-(1-((1-(3-((4-((5-(trifluoromethyl)pyrimidin-2-yl)amino)piperidin-1-yl)sulfonyl)phenyl)piperidin-4-yl)methyl)piperidin-4-yl)-1H-indol-3-yl)piperidine-2,6-dione;
1-(6-(1-((1-(3-((4-((5-cyclopropoxypyrimidin-2-yl)amino)piperidin-1-yl)sulfonyl)phenyl)piperidin-4-yl)methyl)piperidin-4-yl)-1-methyl-1H-indazol-3-yl)-dihydropyrimidine-2,4(1H,3H)-dione;
1-(6-(1-((1-(3-((4-((5-isopropoxypyrimidin-2-yl)amino)piperidin-1-yl)sulfonyl)-phenyl)piperidin-4-yl)methyl)piperidin-4-yl)-1-methyl-1H-indazol-3-yl)dihydropyrimidine-2,4(1H,3H)-dione;
3-(6-(1-((1-(3-((4-((5-chloropyrimidin-2-yl)amino)piperidin-1-yl)sulfonyl)-phenyl)piperidin-4-yl)methyl)piperidin-4-yl)-1-methyl-1H-indol-3-yl)piperidine-2,6-dione;
(R)-4-((4-((5-(difluoromethoxy)pyrimidin-2-yl)amino)piperidin-1-yl)sulfonyl)-2-(2-((4-(3-(2,4-dioxotetrahydropyrimidin-1(2H)-yl)-1-methyl-1H-indazol-6-yl)piperidin-1-yl)methyl)pyrrolidin-1-yl)benzonitrile;
(S)-4-((4-((5-(difluoromethoxy)pyrimidin-2-yl)amino)piperidin-1-yl)sulfonyl)-2-(2-((4-(3-(2,4-dioxotetrahydropyrimidin-1(2H)-yl)-1-methyl-1H-indazol-6-yl)piperidin-1-yl)methyl)pyrrolidin-1-yl)benzonitrile;
(S)-1-(6-(1-((1-(3-((4-((5-(difluoromethoxy)pyrimidin-2-yl)amino)piperidin-1-yl)sulfonyl)phenyl)piperidin-3-yl)methyl)piperidin-4-yl)-1-methyl-1H-indazol-3-yl)dihydropyrimidine-2,4(1H,3H)-dione;
(R)-1-(6-(1-((1-(3-((4-((5-(difluoromethoxy)pyrimidin-2-yl)amino)piperidin-1-yl)sulfonyl)phenyl)piperidin-3-yl)methyl)piperidin-4-yl)-1-methyl-1H-indazol-3-yl)dihydropyrimidine-2,4(1H,3H)-dione;
(S)-1-(6-(1-((1-(3-((4-((5-(difluoromethoxy)pyrimidin-2-yl)amino)piperidin-1-yl)sulfonyl)phenyl)pyrrolidin-2-yl)methyl)piperidin-4-yl)-1-methyl-1H-indazol-3-yl)dihydropyrimidine-2,4(1H,3H)-dione;
(R)-1-(6-(1-((1-(3-((4-((5-(difluoromethoxy)pyrimidin-2-yl)amino)piperidin-1-yl)sulfonyl)phenyl)piperidin-2-yl)methyl)piperidin-4-yl)-1-methyl-1H-indazol-3-yl)dihydropyrimidine-2,4(1H,3H)-dione;
(S)-1-(6-(1-((1-(3-((4-((5-(difluoromethoxy)pyrimidin-2-yl)amino)piperidin-1-yl)sulfonyl)phenyl)piperidin-2-yl)methyl)piperidin-4-yl)-1-methyl-1H-indazol-3-yl)dihydropyrimidine-2,4(1H,3H)-dione;
1-(6-(4-(((3-((4-((5-chloropyrimidin-2-yl)amino)piperidin-1-yl)sulfonyl)benzyl)amino)methyl)piperidin-1-yl)-1-methyl-1H-indazol-3-yl)dihydropyrimidine-2,4(1H,3H)-dione;
1-(6-(4-((3-((4-((5-chloropyrimidin-2-yl)amino)piperidin-1-yl)sulfonyl)benzyl)(methyl)amino)piperidin-1-yl)-1-methyl-1H-indazol-3-yl)dihydropyrimidine-2,4(1H,3H)-dione;
1-(1-methyl-6-(1-(3-((4-((5-(trifluoromethoxy)pyrimidin-2-yl)amino)piperidin-1-yl)sulfonyl)benzyl)piperidin-4-yl)-1H-indazol-3-yl)dihydropyrimidine-2,4(1H,3H)-dione;
1-(1-methyl-6-(1-(1-(1-((4-((5-(trifluoromethyl)pyrimidin-2-yl)amino)piperidin-1-yl)sulfonyl)piperidin-3-yl)propan-2-yl)piperidin-4-yl)-1H-indazol-3-yl)dihydropyrimidine-2,4(1H,3H)-dione;
1-(6-(1-(3-((4-((5-(chlorodifluoromethoxy)pyrimidin-2-yl)amino)piperidin-1-yl)sulfonyl)benzyl)piperidin-4-yl)-1-methyl-1H-indazol-3-yl)dihydropyrimidine-2,4(1H,3H)-dione;
1-(7-chloro-6-(1-(3-((4-((5-chloropyrimidin-2-yl)amino)piperidin-1-yl)sulfonyl)benzyl)piperidin-4-yl)-1-methyl-1H-indazol-3-yl)dihydropyrimidine-2,4(1H,3H)-dione; and
1-(6-(1-(3-(3-((4-((5-(difluoromethoxy)pyrimidin-2-yl)amino)piperidin-1-yl)sulfonyl)phenyl)-2,2-dimethylpropyl)piperidin-4-yl)-1-methyl-1H-indazol-3-yl)dihydropyrimidine-2,4(1H,3H)-dione;
or a pharmaceutically acceptable salt thereof.

54. A pharmaceutical composition comprising a compound of any one of claims 1 to 53, or a pharmaceutically acceptable salt thereof, and a pharmaceutically acceptable excipient.

55. A method of treating a disease mediated by CDK2 in a patient which method comprises administering to the patient in recognized need thereof, a therapeutically effective amount of a compound of any one of claims 1 to 53, or a pharmaceutical composition of claim 53.

56. A method of treating cancer in a patient which method comprises administering to the patient in need thereof, a therapeutically effective amount a compound of any one of claims 1 to 53, or a pharmaceutically acceptable salt thereof, or a pharmaceutical composition of claim 54.

57. The method of claim 56, wherein the compound of any one of claims 1 to 53 or a pharmaceutically acceptable salt thereof, or a pharmaceutical composition of 54 is administered in combination with at least one other anticancer agent.

58. The method of claim 56 or 57, wherein the cancer is lung cancer, skin cancer, bladder cancer, breast cancer, cervical cancer, colorectal cancer, cancer of the small intestine, colon cancer, rectal cancer, cancer of the anus, endometrial cancer, gastric cancer, head and neck cancer, liver cancer, ovarian cancer, prostate cancer, testicular cancer, uterine cancer, esophageal cancer, gall bladder cancer, pancreatic cancer, stomach cancer, thyroid cancer, or parathyroid cancer.
